(19) United States
(12) Patent Application Publication
(10) Pub. No.: US 2026/0216345 A1
(43) Pub. Date: Jul. 30, 2026

(54) M6PR BINDING COMPOUNDS AND CONJUGATES

(71) Applicant: Lycia Therapeutics, Inc., South San Francisco, CA (US)

(72) Inventors: Jason G. Lewis, Castro Valley, CA (US); Eric D. Turtle, Belmont, CA (US)

(21) Appl. No.: 19/148,575

(22) PCT Filed: Jan. 17, 2024
(86) PCT No.: PCT/US2024/011899

§ 371 Date: Jul. 16, 2025

Related U.S. Application Data
(60) Provisional application No. 63/439,806, filed on Jan. 18, 2023.

(51) Int. Cl. A61K 47/54; C07H 15/10; C07H 15/203; C07H 15/22; C07H 15/26

(52) U.S. Cl. CPC A61K 47/549; A61K 47/555; C07H 15/10; C07H 15/203; C07H 15/22; C07H 15/26

(57) ABSTRACT

The present disclosure provides a class of compounds including a ligand moiety that specifically binds to a cell surface mannose-6-phosphate receptor (M6PR). The M6PR binding compounds can trigger the receptor to internalize into the cell a bound compound. The ligand moieties of this disclosure can be linked to a variety of moieties of interest without impacting the specific binding to, and function of, the M6PR. Also provided are compounds that are conjugates of the ligand moieties linked to a biomolecule, such as an antibody, which conjugates can harness cellular pathways to remove specific target proteins from the cell surface or the extracellular milieu. For example, the conjugates described herein may sequester and/or degrade a target molecule of interest in a cell's lysosome. Also provided are methods of using the conjugates to target a protein for sequestration and/or lysosomal degradation.

### CROSS REFERENCE TO RELATED APPLICATIONS

This application claims the benefit under 35 U.S.C. § 119(e) to U.S. Provisional Application No. 63/439,806, filed Jan. 18, 2023, which is hereby incorporated by reference in its entirety.

### 1. BACKGROUND

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

Mannose-6-phosphate is a monosaccharide ligand that plays a key role in the intracellular retention and secretion of lysosomal hydrolytic enzymes to which they are attached. When this sugar residue is incorporated onto newly synthesized enzymes it can direct their transport from the Golgi apparatus to the lysosomes where they are active. Membrane-bound, cell surface mannose-6-phosphate receptors (M6PR's) play a role in many biological processes, including the secretion and internalization of such lysosomal enzymes. Endocytosis by an M6PR allows for the internalization into the cell of compounds bearing a mannose 6-phosphate (M6P) ligand and trafficking to lysosomes.

Alternative ligands that provide for binding to cell surface M6PRs followed by transport across cell membranes are of great interest.

### 2. SUMMARY

The present disclosure provides a class of compounds including a ligand moiety that specifically binds to a cell surface mannose-6-phosphate receptor (M6PR). The cell surface M6PR binding compounds can trigger the receptor to internalize into the cell a bound compound. The ligand moieties of this disclosure can be linked to a variety of moieties of interest without impacting the specific binding to, and function of, the cell surface M6PR. Also provided are compounds that are conjugates of the ligand moieties linked to a biomolecule, such as an antibody, which conjugates can harness cellular pathways to remove specific proteins of interest from the cell surface or from the extracellular milieu. For example, the conjugates described herein may sequester and/or degrade a target molecule of interest in a cell's lysosome. Also provided herein are compositions comprising such conjugates and methods of using the conjugates to target a polypeptide of interest for sequestration and/or lysosomal degradation, and methods of using the conjugates to treat disorders or disease.

### 3. BRIEF DESCRIPTION OF THE DRAWINGS

FIG. 1 shows a representative native mass spectrometry MS analysis of an exemplary conjugate, matuzumab-(Compound A) conjugate versus deglycosylated matuzumab.

FIG. 2 shows a representative native mass spectrometry MS analysis of an exemplary conjugate, matuzumab-(Compound 520 (I-7)) conjugate versus deglycosylated matuzumab.

FIG. 3 shows time course activity of cetuximab-(Compound A) and cetuximab-(Compound 520 (I-7)) conjugates on surface EGFR levels in Hela parental and M6PR knockout (KO) cells as measured by surface staining.

FIG. 4 shows time course activity of matuzumab-(Compound A) and matuzumab-(Compound 520 (I-7)) conjugates on surface EGFR levels in Hela parental and M6PR KO cells as measured by surface staining.

FIG. 5 shows in-cell Western blotting images illustrating a dose response of cetuximab-(Compound A), cetuximab-(Compound 520 (I-7)), matuzumab-(Compound A), and matuzumab-(Compound 520 (I-7)) conjugates on total EGFR levels in Hela parental and M6PR KO cells.

FIG. 6 shows time course activity of cetuximab-(Compound A), cetuximab-(Compound 520 (I-7)), matuzumab-(Compound A), and matuzumab-(Compound 520 (I-7)) conjugates on relative EGFR normalized levels in Hela parental and M6PR KO cells.

FIGS. 7A-7F show M6PR binding affinities curves for various exemplary conjugates of fluorescently labeled matuzumab (mtz) or human IgG isotype antibody (isotype) ([ab]): unlabeled control (FIG. 7A), Compound 520 (I-7) (FIG. 7B), Compound 602 (I-8) (FIG. 7C), Compound 603 (I-9) (FIG. 7D), Compound 605 (I-11) (FIG. 7E) and Compound 716 (I-12) (FIG. 7F) to M6PR. Binding to M6PR was determined by ELISA. Conjugates of Compound 520 (I-7) (m or DAR=8) and Compound 605 (I-11) (m or DAR=4) showed the highest and lowest binding affinity, respectively. d4 is DAR 4. d8 is DAR 8. RFU is relative fluorescence units.

FIGS. 8A-8C illustrate serum pharmacokinetic (PK) analysis of exemplary conjugates of an rIgG1 (anti-IgG2a) antibody in mice. Intracellular levels of conjugates of Compound 520 (I-7) (d8 is DAR=8) and (d4 is DAR=4) (FIG. 8A), Compound 604 (I-10) and Compound 605 (I-11) (FIG. 8B), and Compound 603 (I-9) and Compound 716 (I-12) (FIG. 8C) in mouse serum were measured at 0.5, 1, 2, 6, and 24 hours post administration using ELISA. UNLB is an antibody control.

FIG. 9 shows intracellular uptake of exemplary anti-IgG2a conjugates and bound target protein over time in Jurkat cells. Conjugates were detected via fluorescent Alexa488-conjugated target IgG2a-antibody, and intracellular levels of fluorescence (MFI) were determined using FACS after 1 hour and 24 hour.

FIG. 10 illustrates relative intracellular uptake of 10 nM exemplary anti-IgG2a conjugates and bound target protein (Alexa488-conjugated target IgG2a-antibody) into Jurkat cells after 24 hour as a percentage of the uptake of the reference Compound 520 (I-7) (d8 is DAR=8) conjugate.

FIG. 11 a graph of results of a M6PR binding assay for a variety of antibody conjugates of exemplary compounds with various DAR loadings.

FIG. 12 a graph of cell fluorescence (MFI) versus antibody conjugate concentration ([Ab]) indicating that exemplary M6PR binding antibody conjugates exhibited robust uptake of target protein into Jurkat cells after one hour incubation.

FIG. 13 shows a graph of cell fluorescence (MFI) versus antibody conjugate concentration ([Ab]) indicating that various antibody conjugates of exemplary M6PR or ASGPR binding compounds exhibited comparable robust uptake into HepG2 cells after one hour incubation.

FIG. 14 shows a graph demonstrating CI-M6PR dependent cell uptake of exemplary antibody conjugates bound to Alexa488 labeled-IgE target in wild type (WT) K562 cells versus CI-M6PR knockout (KO) cells.

FIG. 15 shows a graph of cellular uptake of various conjugates of omalizumab (anti-IgE) with exemplary M6PR binding compounds, the conjugate bound to Alexa488 labeled-target IgE, in Jurkat cells.

FIG. 16 shows a graph illustrating comparisons of the cellular uptake activity of particular exemplary conjugates from the graph of FIG. 15.

FIG. 17 shows a graph illustrating comparisons of the cellular uptake activity of particular exemplary conjugates from the graph of FIG. 15.

FIG. 18 shows a graph of cellular uptake of various conjugates of omalizumab (anti-IgE) with exemplary M6PR ligand-linkers, bound to Alexa488 labeled-target IgE in Jurkat cells.

FIG. 19 shows a graph illustrating comparisons of the cellular uptake activity of particular exemplary conjugates from the graph of FIG. 18.

FIG. 20 shows a graph illustrating comparisons of the cellular uptake activity of particular exemplary conjugates from the graph of FIG. 18.

FIG. 21 shows a graph illustrating comparisons of the cellular uptake activity of particular exemplary conjugates from the graph of FIG. 18.

FIG. 22 shows a graph of M6PR binding affinity data for various exemplary cetuximab (anti-EGFR) conjugates of this disclosure.

FIG. 23 shows a graph illustrating the cellular uptake activity of particular exemplary target binding conjugates of this disclosure.

FIG. 24 shows a synthetic scheme for a M6PR binding moiety suitable for attachment to a linker and/or moiety of interest.

FIG. 25 shows a synthetic scheme for a M6PR binding moiety suitable for attachment to a linker and/or moiety of interest.

### 4. DETAILED DESCRIPTION

As summarized above, this disclosure provides a class of compounds including a particular ligand moiety, X, that specifically binds to a cell surface mannose-6-phosphate receptor (M6PR), also referred to as a M6PR-binding moiety or M6PR ligand moiety). The M6PR-binding moieties of this disclosure can be linked to a variety of moieties of interest without impacting the specific binding to, and function of, the cell surface M6PR. The inventors have demonstrated that compounds of this disclosure can utilize the functions of cell surface M6PRs in a biological system, e.g., for internalization and/or sequestration to the lysosome of a cell, and in some cases subsequent lysosomal degradation of a target molecule. The compounds of this disclosure find use in a variety of applications. In some embodiments, the M6PR-binding moiety X provides for intracellular delivery of moieties of interest. In some embodiments, the compounds are bifunctional compounds including the M6PR-binding moiety X, linked to a target-binding moiety, for internalization and/or lysosomal degradation of a bound target molecule.

Accordingly, this disclosure provides compounds of formula (XI) including one or more M6PR-binding moieties linked to a moiety of interest Y:

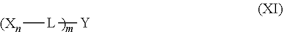

or a salt thereof, wherein:
X is a M6PR-binding moiety (e.g., as described herein);
n is 1 to 500 (e.g., X is linked via a monovalent or multivalent linker, as described herein);
m is 1 to 500 (e.g., 1 to 100, or 1 to 10);
L is a linker; and
Y is a moiety of interest (e.g., as described herein).

The compounds and conjugates, and methods of this disclosure are described in greater detail below. A particular class of M6PR binding compounds is described. In some embodiments, the compounds are biomolecule conjugates that include one or more linked M6PR-binding moieties. Linkers (L) and moieties of interest (Y) which find use in the M6PR binding compounds, and the biomolecule conjugates are also described. Methods in which the compounds and conjugates of this disclosure find use are also described.

### 4.1. M6PR Binding Moiety

As summarized above, the M6PR binding moieties (also referred to as M6PR ligand moieties) of this disclosure can be linked to a variety of moieties of interest without impacting the specific binding to, and function of, the cell surface M6PR. The inventors have demonstrated that M6PR binding moieties having particular structures described below provide for high affinity binding to cell surface M6PRs, and when configured via a linker according to the bifunctional compounds of this disclosure can utilize the functions of cell surface M6PRs in a biological system, e.g., for internalization, and/or degradation of a target molecule.

The terms “mannose-6-phosphate receptor” and “M6PR” refer to receptors of the family of mannose-6-phosphate receptors. M6PRs are transmembrane glycoprotein receptors that target enzymes to lysosomes in cells. MP6R endogenously transports proteins bearing N-glycans capped with mannose-6-phosphate (M6P) residues to lysosomes, and cycles between endosomes, the cell surface, and the Golgi complex. See, e.g., Ghosh et al., Nat. Rev. Mol. Cell Biol. 2003; 4: 202-213. The family of M6PRs includes the cation independent mannose-6-phosphate receptor (CI-M6PR). The CI-M6PR is also referred to as the insulin-like growth factor 2 receptor (IGF2R) and is encoded in humans by the IGF2R gene (see, e.g., NCBI Reference Sequence: NM_000876.3, and NCBI Gene ID: 3482). The CI-M6PR binds insulin-like growth factor 2 (IGF-2) and mannose-6-phosphate (M6P)-tagged proteins. The compounds of this disclosure can specifically bind to a cell surface M6PR, for example, an internalizing CI-M6PR cell surface receptor. In particular embodiments, the surface CI-M6PR is a human CI-M6PR. It is understood that the terms M6PR and CI-M6PR are used interchangeably when referring to the binding properties of the M6PR binding moieties and compounds of this disclosure.

A compound comprising such M6PR binding moiety (X) (e.g., as described herein), may bind to other receptors, for example, may bind with lower affinity as determined by, e.g., immunoassays or other assays known in the art. In a specific embodiment, X, or a compound as described herein including such X specifically binds to a cell surface CI-M6PR with an affinity that is at least 2 logs, 2.5 logs, 3 logs, 4 logs or greater than the affinity when X or the compound bind to another cell surface receptor. In a specific embodiment, X, or a compound as described herein comprising X, specifically binds to CI-M6PR with an affinity (K_{d}) 20 mM or less. In particular embodiments, such binding is with an affinity (K_{d}) is 10 mM or less, 1 mM or less, 100 uM or less, 10 uM or less, 1 uM or less, 100 nM or less, 10 nM or less, or 1 nM or less. The terms “binds,” “binds to,” “specifically binds” or “specifically binds to” in this context are used interchangeably.

The M6PR binding compounds of this disclosure include a moiety (X) (e.g., as described herein) which is a D-mannopyranose analog that specifically binds to the cell surface receptor M6PR. The M6PR binding compounds can be monovalent or multivalent (e.g., bivalent or trivalent or of higher valency), where a monovalent compound includes a single M6PR ligand moiety, and a monovalent compound includes two or more such moieties.

### 4.1.1. Alpha-Linked Pyranose Ring

The M6PR binding moiety of the compounds of this disclosure can include a linked pyranose ring described by formula (II):

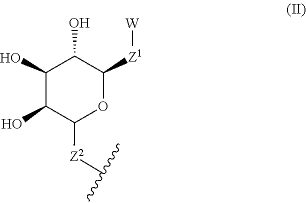

where:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

In some embodiments of formula (II), Z² is a linking moiety connected to the pyranose sugar ring at the anomeric or 1-position with an alpha-configuration as shown in formula (IIa) below:

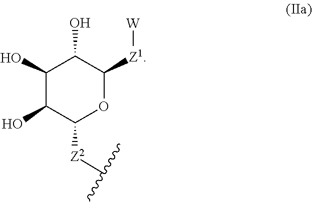

### 4.1.2. Beta-Linked Pyranose Ring

The inventors have demonstrated that although M6PR binding compounds having a M6PR binding moiety with an anomeric alpha-configuration of formula (IIa) can provide good binding and internalization activity at the receptor, in some cases it is possible to impart more potent binding and internalization activity at the M6PR by configuring the central pyranose sugar ring of the M6PR binding moiety with a beta-configuration at the anomeric position. In some embodiments, such M6PR binding moieties can provide for increased stability at the pyranose ring.

Accordingly, in some embodiments of formula (II), Z² is a linking moiety connected to the sugar ring at the anomeric or 1-position with a beta-configuration as shown in formula (IIb) below:

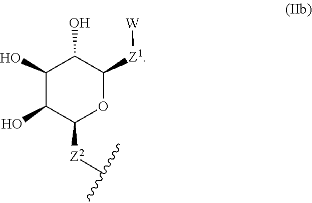

### 4.2. M6PR Binding Compounds

Although moieties of formula (II) can exhibit binding activity for the M6PR, the inventors have demonstrated that when particular types of cyclic groups are linked with a particular configuration adjacent to the pyranose ring of formula (II) via the linking moiety Z², a M6PR binding moiety of desirable binding activity can be produced.

Accordingly, in some embodiments of formula (II), the M6PR binding moiety (X) can be described by formula (III):

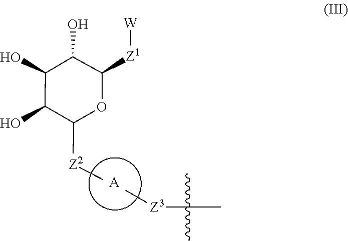

or a prodrug thereof, or a salt thereof, wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
A is independently an optionally substituted cyclic group; and
Z³ is independently a linking moiety.

In some embodiments of formula (II)-(III), W is a non-hydrolyzable hydrophilic head group.

In some embodiments of formula (II)-(III), Z² is optionally substituted ethylene. In some embodiments of formula (II)-(III), Z² is optionally substituted ethenylene.

In some embodiments of formula (II)-(III), Z² is O. In some embodiments of formula (II)-(III), Z² is S. In some embodiments of formula (II)-(III), Z² is —NR²¹—. In some embodiments of formula (II)-(III), Z² is —C(R²²)₂—, wherein each R²² is independently selected from H, halogen (e.g., F) and optionally substituted (C₁-C₆)alkyl. In some embodiments of formula (II)-(III), Z² is —CH₂—.

In some embodiments of formula (II)-(III), A is optionally substituted aryl, optionally substituted heteroaryl, optionally substituted heterocycle, or optionally substituted cycloalkyl. In some embodiments of formula (II)-(III), A is independently an optionally substituted aryl or heteroaryl linking moiety (e.g., monocyclic or bicyclic aryl or heteroaryl, optionally substituted).

Exemplary Z³ linking moieties of formula (II)-(III) are described herein.

Such M6PR-binding moieties of formula (III) can be attached to a moiety or molecule of interest to produce a bifunctional compound that undergoes effective M6PR-mediated cell internalization. The inventors have further demonstrated that when the moiety or molecule of interest is a target protein-binding moiety, the M6PR binding compound also provides for M6PR mediated internalization and/or degradation of bound target protein.

Accordingly, in some embodiments of formula (XI), the M6PR binding compound is of formula (XII):

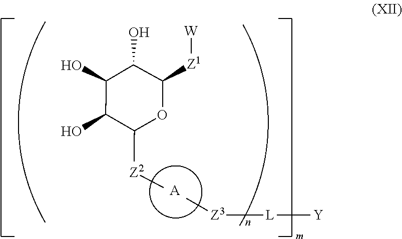

or a prodrug thereof, or a salt thereof,
wherein:
   W is a hydrophilic head group;
   Z¹ is selected from optionally substituted (C₁-C₃)alkylene and optionally substituted ethenylene;
   Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
   A is independently an optionally substituted cyclic group;
   Z³ is independently a linking moiety;
   n is 1 to 500;
   L is a linker;
   Y is a moiety of interest; and
   m is 1 to 100.

In some embodiments of formula (XI)-(XII), m is 1, and the cell surface M6PR binding compound is of formula (XIII):

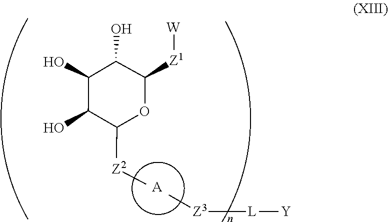

or a prodrug thereof, or a salt thereof,
wherein:
   W is a hydrophilic head group;
   Z¹ is selected from optionally substituted (C₁-C₃)alkylene and optionally substituted ethenylene;
   Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
   A is independently an optionally substituted cyclic group;
   Z³ is independently a linking moiety;
   n is 1 to 500;
   L is a linker; and
   Y is a moiety of interest (e.g., as described herein).

In some embodiments of formula (XIII), Y is a chemoselective ligation group. In some embodiments of formula (XIII), n is 1. In some embodiments of formula (XIII), Y is a chemoselective ligation group connected to “n” M6PR binding moieties (Xn−) via a single linker -L-. In some embodiments of formula (XIII), n is 2, 3, 4, or 5. In some embodiments of formula (XIII), n is 5-10. In some embodiments of formula (XIII), n is 10-100, such as 20-80, or 20-50. In some embodiments of formula (XIII), when n is 5 or more, then L is a polypeptide containing linker (e.g., as described herein).

In some embodiments of formula (XII)-(XIII), when n is 1 and A is phenyl, then: i) L comprises a backbone of at least 16 consecutive atoms (e.g., at least 18 consecutive atoms, or at least 20 consecutive atoms, in some cases up to about 200 consecutive atoms); ii) Y is a biomolecule; and/or ii) Z³ is amide, sulfonamide, urea or thiourea linking moiety to linker L.

In some embodiments of formula (XII), Z² is a linking moiety connected to the sugar ring at the anomeric or 1-position with an alpha-configuration as shown in formula (IIa) such that the compound is of formula (XIIa):

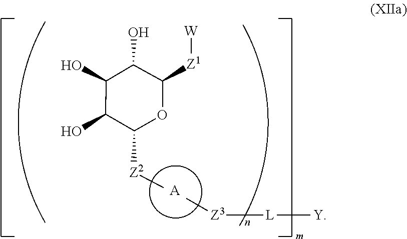

In some embodiments of formula (XII), Z² is a linking moiety connected to the sugar ring at the anomeric or 1-position with a beta-configuration as shown in formula (IIb), such that the compound is of formula (XIIb):

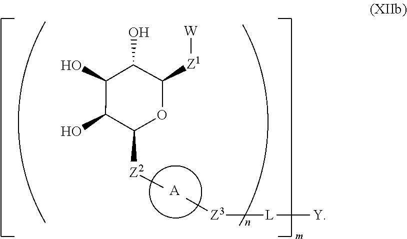

In some embodiments of formula (XI)-(XIIb), multiple M6PR binding moieties, e.g., of formula (III), are linked via multiple linkers L to different ligation sites on a moiety of interest Y. In some embodiments, when Y is a biomolecule, the compound of formula (XI)-(XIIb) can be referred to as a conjugate.

### 4.2.1. Hydrophilic Head Group and Linking Moieties

In some embodiments of formula (II)-(XIII), the M6PR binding moiety (X) includes an analog of a D-mannopyranose ring, with a hydrophilic head group, or a precursor or prodrug thereof, that is connected via a linking moiety (Z′) to the 5-position of the sugar ring. The linking moiety can be of 1-6 atoms in length, such as 1-5, 1-4 or 1-3 atoms in length, e.g., 1 or 2 atoms in length. It is understood that the length of the linking moiety can be selected in conjunction with the hydrophilic head group.

The hydrophilic head group (W) can be any suitable negatively charged group, or salt thereof. In some embodiments, the hydrophilic head group is a neutral, polar, hydrophilic group. In general, the hydrophilic head group is capable of hydrogen bonding or electrostatic interactions with the M6PR, under aqueous or physiological conditions, similar to those of the phosphate group of M6P. The hydrophilic head group can be a bioisostere (e.g., a structural or functional mimic) of the 6-phosphate group of the naturally occurring mannose-6-phosphate ligand. In some embodiments, the hydrophilic head group is non-hydrolyzable, i.e., a functional group that is stable against its cleavage (e.g., chemically or enzymatically) under physiological conditions, from the Z¹ linking moiety and/or pyranose ring of X to which the hydrophilic head group is attached.

The hydrophilic head group is generally a small group, such as a heteroatom containing functional group, or single heterocyclic ring, and in some cases has a MW of less than 200, such as less than 150, or less than 100.

In some embodiments, the hydrophilic head group is a phosphonate, or a bioisostere thereof, such as a carboxylate or malonate. In some embodiments, the hydrophilic head group is a thiophosphonate.

In some embodiments of formula (II)-(XIII), the hydrophilic head group is not a phosphate, thiophosphate or dithiophosphate, as such groups would have phosphate ester linkages to the compound which can be unstable and susceptible to cleavage under physiological conditions (e.g., by phosphatases in a biological system or chemically). For example, the 6-phosphate ester group of M6P exhibits undesirable stability as compared to a phosphonate analog, or other more stable head group. This disclosure provides alternative non-hydrolyzable head groups in addition to phosphonate which retain binding and internalization activity of the resulting M6PR binding compound.

In any one of the embodiments of formula (II)-(XIII), the hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

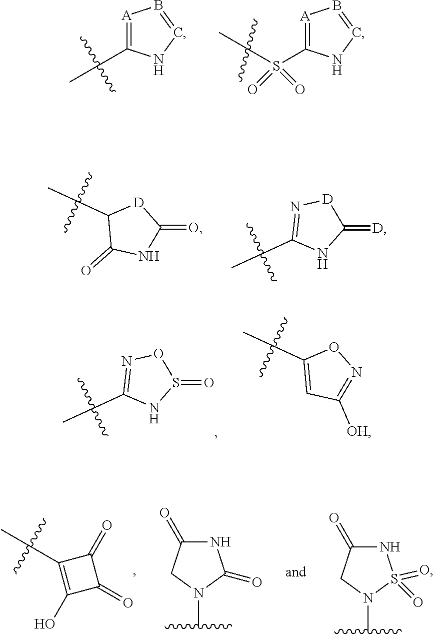

or a salt thereof,
wherein:
   R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
   A, B, and C are each independently CH or N; and
   D is each independently O or S.

In some embodiments of formula (II)-(XIII), the hydrophilic head group W is phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof. In some embodiments of formula (II)-(XIII), the hydrophilic head group W is non-hydrolyzable, and accordingly, is not selected from phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof.

In some embodiments of formula (II)-(XIII), the hydrophilic head group W is charged, e.g., capable of forming a salt under aqueous or physiological conditions. In some embodiments of formula (II)-(XIII), the hydrophilic head group W is selected from —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —COOH, —CH(COOH)₂, —CR¹R²COOH, and —NHC(O)CO₂H.

In some embodiments of formula (II)-(XIII), the hydrophilic head group W is phosphonate or thiophosphonate (e.g., —P═O(OH)₂, —P═S(OH)₂, —P═O(SH)(OH), or —P═S(SH)(OH), or a salt thereof). In some embodiments of formula (II)-(XIII), the hydrophilic head group W is phosphonate or a salt thereof. In some embodiments of formula (II)-(XIII), the hydrophilic head group W is —CO₂H or a salt thereof. In some embodiments of formula (II)-(XIII), the hydrophilic head group W is malonate (e.g., —CH(COOH)₂ or a salt thereof).

In some embodiments of formula (II)-(XIII), the hydrophilic head group W is selected from —SO₂OH (i.e., —SO₃H), —S(O)OH, —OSO₂OH, and —NHSO₃H. In some embodiments of formula (II)-(XIII), the hydrophilic head group W is sulfonate (e.g., —SO₃H or a salt thereof).

In some embodiments, the hydrophilic head group W is neutral hydrophilic. In some embodiments of formula (II)-(XIII), the hydrophilic head group W is selected from —OH, —CR²R²OH, —CN, —CONH₂, —CONHR³, —CONR³R⁴, —CONH(OH), —CONH(OR³), —CONHSO₂R³, —SO₂R³, —SOR³R⁴, —SO₂NH₂, —SO₂NHR³, —SO₂NR³R⁴, —SO₂NHCOR³, —NHCOR³, —NHSO₂NHR³, —NHC(O)NHS(O)₂R³, and —NHSO₂R³.

In some embodiments of formula (II)-(XIII), the hydrophilic head group W comprises a heterocycle, such as

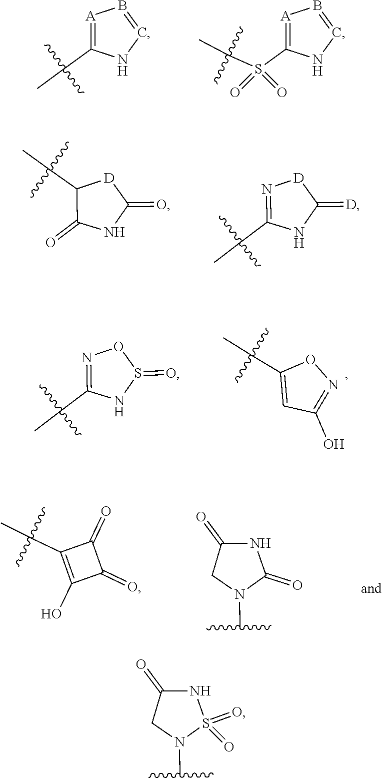

and or a salt thereof,
wherein A, B, and C are each independently CH or N; and D is each independently O or S.

In some embodiments of formula (II)-(XIII), the hydrophilic head group W comprises a 5-membered heterocycle, such as

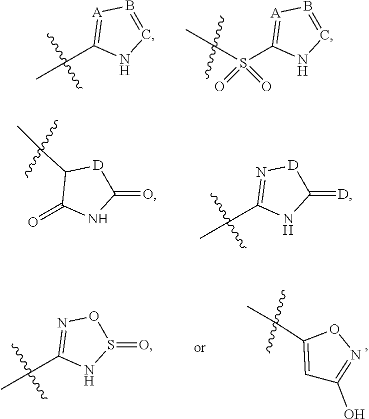

or a salt thereof.

In some embodiments of formula (II)-(XIII), the hydrophilic head group W is linked to the pyranose ring via a Z¹ that is selected from optionally substituted (C₁-C₂)alkylene and optionally substituted ethenylene. The Z¹ can be selected in conjunction with W so as to provide a desired spacing between the 5-position of the ring and the charged or polar center of W. For example, when W is a malonate having a CH atom linking the two carboxylic acid groups, Z¹ can be methylene, which together provide a desirable two carbon spacer between the ring and the COOH groups.

In some embodiments of formula (II)-(XIII), Z¹ is methylene or substituted methylene. In some embodiments of formula (II)-(XIII), Z¹ is ethyl or substituted ethyl. In some embodiments of formula (II)-(XIII), Z¹ is ethenylene or substituted ethenylene. In some embodiments of formula (II)-(XIII), Z¹ is substituted with one or more halogen, e.g., fluoro.

In some embodiments of formula (III), the M6PR binding moiety (X) is described by one of formula (IV-1) to (IV-3):

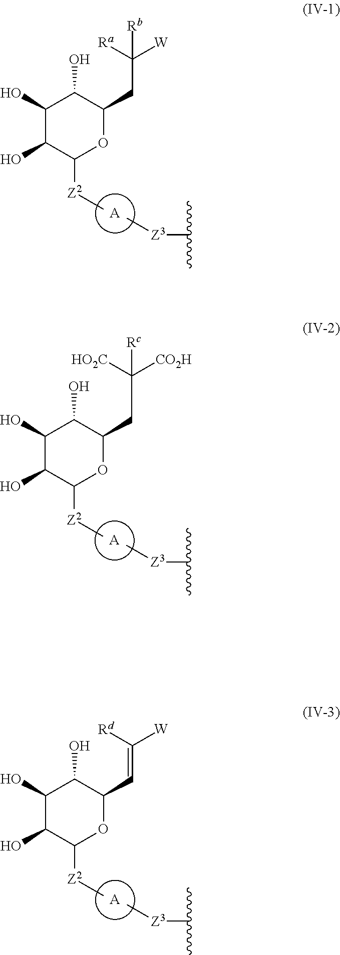

wherein R^{a}, R^{b}, R^{c} and R^{d} are independently H or F.

In some embodiments of formula (IV-1) to (IV-3), Z² is O.

In some embodiments of formula (IV-1) to (IV-3), Z² is S.

In some embodiments of formula (IV)-1 to (IV-3), Z² is —NR²¹—.

In some embodiments of formula (IV-1) to (IV-3), Z² is —C(R²²)₂—, wherein each R²² is independently selected from H, halogen (e.g., F) and optionally substituted (C₁-C₆)alkyl. In some embodiments of formula (IV-1) to (IV-3), Z² is —CH₂—.

In some embodiments of formula (IV-1) to (IV-3), R^{a}, R^{b}, R^{c} and R^{d} are each H.

In some embodiments of formula (IV-1) R^{a} is H and R^{b} is F. In some embodiments of formula (IV-1) R^{a} and R^{b} are each F.

In some embodiments of formula (IV-2) R^{c} is H. In some embodiments of formula (IV-2) R^{c} is F.

In some embodiments of formula (IV-3) R^{d} is H. In some embodiments of formula (IV-3) R^{d} is F.

In some embodiments of formula (IV-1) and (IV-3), W is selected from —P═O(OH)₂, —P═S(OH)₂, —P═O(SH)(OH), —P═S(SH)(OH), and —COOH, or a salt thereof. In some embodiments of formula (IV-1) and (IV-3), W is —P═O(OH)₂, or a salt thereof. In some embodiments of formula (IV-1) and (IV-3), W is COOH, or a salt thereof.

In some embodiments of formula (IV-1) R^{a} and R^{b} are each F, and W is —P═O(OH)₂, or a salt thereof. In some embodiments of formula (IV-1) R^{a} and R^{b} are each H, and W is —P═O(OH)₂, or a salt thereof. In some embodiments of formula (IV-1) R^{a} is F, R^{b} is H, and W is —P═O(OH)₂, or a salt thereof.

In some embodiments of formula (IV-1) to (IV-3), Z² is linked to the anomeric position of the pyranose ring with an alpha-configuration. In such cases, the M6PR binding moiety (X) of (IV-1) to (IV-3) can be referred to as formula (IV-A1) to (IV-A3), respectively.

In some embodiments of formula (IV-A1) to (IV-A3), Z² is S. In some embodiments of formula (IV-A1) to (IV-A3), Z² is 0. In some embodiments of formula (IV-A1) to (IV-A3), Z² is —CH₂—. In some embodiments of formula (IV-A1) to (IV-A3), Z² is —CF₂—.

In some embodiments of formula (IV-A1) and (IV-A3), W is selected from —P═O(OH)₂, —P═S(OH)₂, —P═O(SH)(OH), —P═S(SH)(OH), and —COOH, or a salt thereof. In some embodiments of formula (IV-A1) and (IV-A3), W is —P═O(OH)₂, or a salt thereof. In some embodiments of formula (IV-A1) and (IV-A3), W is COOH, or a salt thereof.

In some embodiments of formula (IV-A1) R^{a} and R^{b} are each F, and W is —P═O(OH)₂, or a salt thereof. In some embodiments of formula (IV-A1) R^{a} and R^{b} are each H, and W is —P═O(OH)₂, or a salt thereof. In some embodiments of formula (IV-A1) R^{a} is F, R^{b} is H, and W is —P═O(OH)₂, or a salt thereof.

In some embodiments of formula (IV-1) to (IV-3), Z² is linked to the anomeric position of the pyranose ring with a beta-configuration. The inventors demonstrated that a compound including a M6PR binding moiety having a β-glycoside configuration can have at least equivalent binding and/or cellular uptake activity as compared to a conjugate having the corresponding α-glycoside configuration. In some embodiments, such M6PR binding moieties having a β-glycoside configuration can provide increased stability as compared to a reference compound having a (3-glycoside configuration. Accordingly, in some embodiments of formula (IV), the M6PR binding moiety (X) is described by one of formula (IV-B1) to (IV-B3):

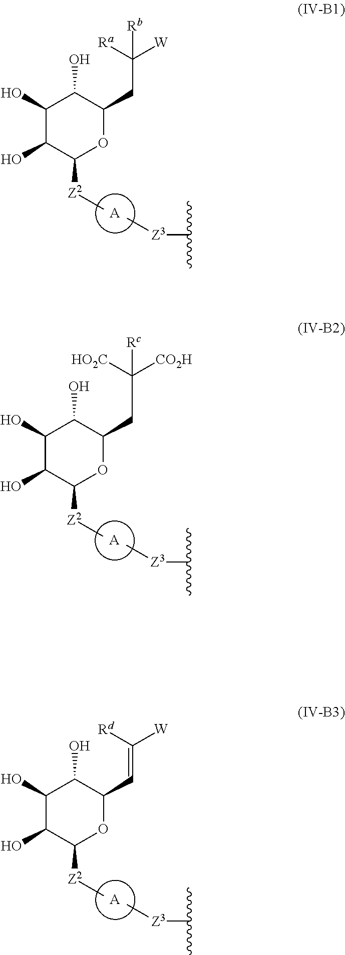

wherein R^{a}, R^{b}, R^{c} and R^{d} are independently H or F.

In some embodiments of formula (IV-B1) to (IV-B3), Z² is S. In some embodiments of formula (IV-B1) to (IV-B3), Z² is 0. In some embodiments of formula (IV-B1) to (IV-B3), Z² is —CH₂—. In some embodiments of formula (IV-B1) to (IV-B3), Z² is —CF₂—.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof. In some embodiments of formula (IV-B1) and (IV-B3), W is —P═O(OH)₂, or a salt thereof. In some embodiments of formula (IV-B1) and (IV-B3), W is COOH, or a salt thereof.

In some embodiments of formula (IV-B1) R^{a} and R are each F, and W is —P═O(OH)₂, or a salt thereof. In some embodiments of formula (IV-B1) R^{a} and R^{b} are each H, and W is —P═O(OH)₂, or a salt thereof. In some embodiments of formula (IV-B1) R^{a} is F, R^{b} is H, and W is —P═O(OH)₂, or a salt thereof.

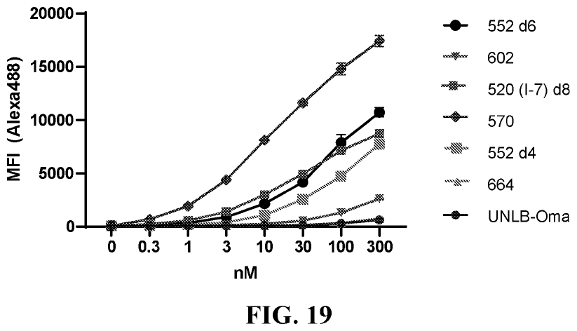

The inventors demonstrated that a conjugate including M6PR binding moiety having a (3-S-glycoside configuration can have at least equivalent or superior binding and/or cellular uptake activity as compared to a conjugate having the corresponding α—S-glycoside configuration, or to a conjugate having an α—O-glycoside configuration. See FIG. 19.

Accordingly, in some embodiments of formula (IV-B1) to (IV-B3), the M6PR binding moiety (X) is described by one of formula (IV-BS1) to (IV-BS3):

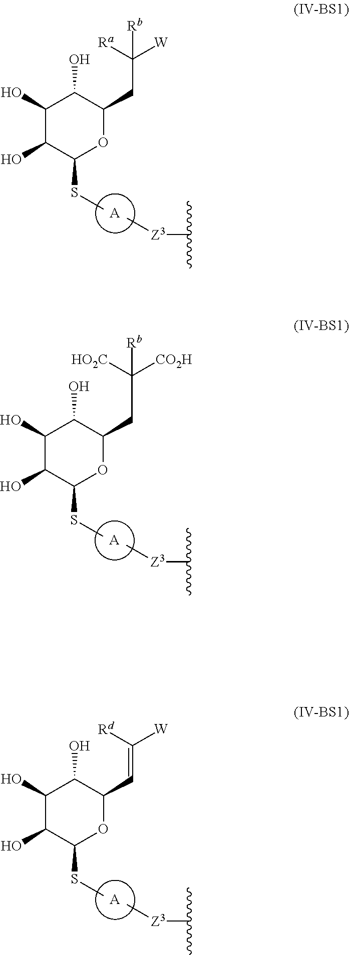

wherein R^{a}, R^{b}, R^{c} and R^{d} are independently H or F.

In some embodiments of formula (IV-BS1) to (IV-BS3), R^{a}, R^{b}, R^{c} and R^{d} are each H.

In some embodiments of formula (IV-BS1) R^{a} is H and R^{b} is F. In some embodiments of formula (IV-BS1) R^{a} and R^{b} are each F.

In some embodiments of formula (IV-BS2) R^{c} is H. In some embodiments of formula (IV-B2) R^{c} is F.

In some embodiments of formula (IV-BS3) R^{d} is H. In some embodiments of formula (IV-BS3) R^{d} is F.

In some embodiments of formula (IV-BS1) to (IV-BS3), Z² is S. In some embodiments of formula (IV-BS1) to (IV-BS3), Z² is 0. In some embodiments of formula (IV-BS1) to (IV-BS3), Z² is —CH₂—. In some embodiments of formula (IV-BS1) to (IV-BS3), Z² is —CF₂—.

In some embodiments of formula (IV-BS1) and (IV-BS3), W is selected from —P═O(OH)₂, —P═S(OH)₂, —P═O(SH)(OH), —P═S(SH)(OH), and —COOH, or a salt thereof. In some embodiments of formula (IV-BS1) and (IV-BS3), W is —P═O(OH)₂, or a salt thereof. In some embodiments of formula (IV-BS1) and (IV-BS3), W is COOH, or a salt thereof.

In some embodiments of formula (IV-BS1) R^{a} and R^{b} are each F, and W is —P═O(OH)₂, or a salt thereof. In some embodiments of formula (IV-BS1) R^{a} and R^{b} are each H, and W is —P═O(OH)₂, or a salt thereof. In some embodiments of formula (IV-BS1) R^{a} is F, R^{b} is H, and W is —P═O(OH)₂, or a salt thereof.

In some embodiments, the mannose ring or analog thereof of the M6PR binding moiety can be incorporated into the compounds of this disclosure by attachment of a linking moiety to the Z² group attached at the anomeric or 1-position of the sugar ring.

In some embodiments, the M6PR binding moiety is incorporated into the compounds of this disclosure by attachment of a linker to the Z³ group attached to the cyclic group A. It is understood that in the compounds of formula (III), the cyclic group attached to Z² can be considered part of the M6PR binding moiety (X) and provide for a desirable binding property to the M6PR.

### 4.2.2. Cyclic Group A

The A cyclic group of formula (III)-(XIII) can be a monocyclic or bicyclic group. A bicyclic group of interest can be a fused bicyclic group or a bicyclic group containing two monocyclic linked via a covalent bond. The A cyclic group of formula (III)-(XIII) can be optionally substituted aryl, optionally substituted heteroaryl, optionally substituted heterocycle (e.g., saturated heterocycle), or optionally substituted cycloalkyl.

The A cyclic group of formula (III)-(XIII) can be a monocyclic aryl or monocyclic heteroaryl group. In some embodiments of formula (III)-(XIII), A is a 5-membered monocyclic heteroaryl group. In some embodiments of formula (III)-(XIII), A is a 6-membered monocyclic aryl or heteroaryl group. In some embodiments of formula (III)-(XIII), A can be a multicyclic aryl or multicyclic heteroaryl group, such as a bicyclic aryl or bicyclic heteroaryl group. In some embodiments of formula (III)-(XIII), A is a fused bicyclic group. In some embodiments of formula (III)-(XIII), A is a bicyclic group comprising two aryl and/or heteroaryl monocyclic rings connected via a covalent bond. In some embodiments of formula (III)-(XIII), A is a bicyclic aryl or bicyclic heteroaryl group having two 6-membered rings. In some embodiments of formula (III)-(XIII), A is a bicyclic aryl or bicyclic heteroaryl group having one 6-membered ring that is connected via a covalent bond or fused to a 5-membered ring.

In some embodiments of formula (III)-(XIII), A is selected from optionally substituted phenyl, optionally substituted pyridyl, optionally substituted biphenyl, optionally substituted naphthalene, optionally substituted quinoline, optionally substituted triazole and optionally substituted phenylene-triazole.

In some embodiments of formula (III)-(XIII), A is not phenyl (also referred to as phenylene in the context of formula (III), e.g., 1,4-phenylene).

In some embodiments of formula (III)-(XIII), A is substituted with at least one OH substituent. In some embodiments of formula (III)-(XIII), A is substituted with 1, 2, or more OH groups. In some embodiments of formula (III)-(XIII), A is substituted with at least one optionally substituted (C₁-C₆)alkyl.

In some embodiments of formula (III)-(XIII), A is optionally substituted 1,4-phenylene, optionally substituted 1,3-phenylene, or optionally substituted 2,5-pyridylene.

In some embodiments of formula (III)-(XIII), A is selected from:

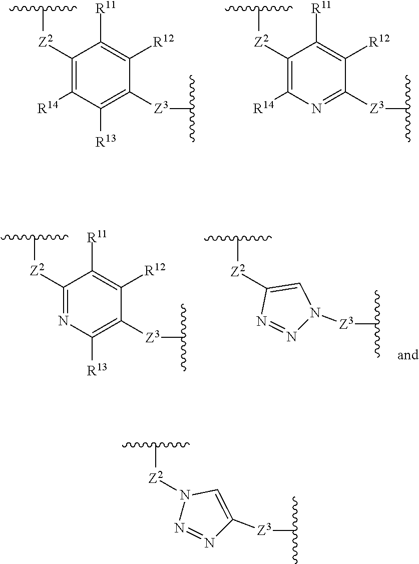

wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and R²⁵ is independently selected from H, and optionally substituted (C₁-C₆)alkyl.

In some embodiments of formula (III)-(XIII), A is optionally substituted fused bicyclic aryl or optionally substituted fused bicyclic heteroaryl.

In some embodiments of formula (III)-(XIII), A is optionally substituted naphthalene or optionally substituted quinoline.

In some embodiments of formula (III)-(XIII), A is selected from:

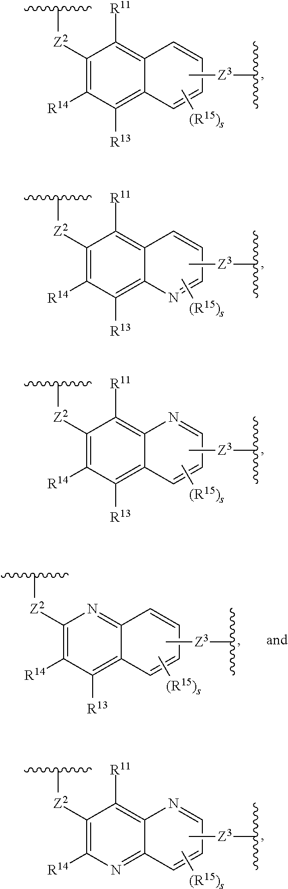

wherein:
R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

In some embodiments of formula (III)-(XIII), A is selected from:

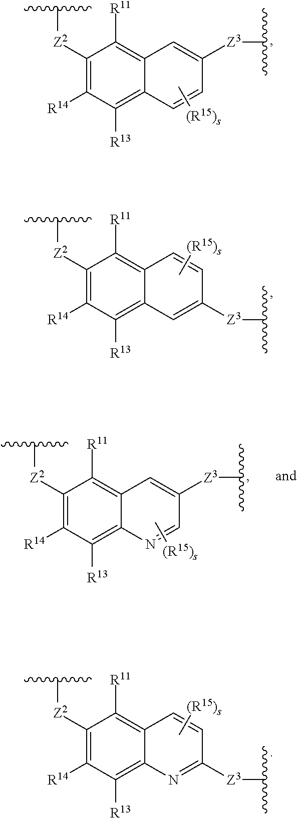

In some embodiments of formula (III)-(XIII), A is optionally substituted bicyclic aryl or optionally substituted bicyclic heteroaryl of following formula:

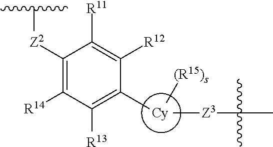

or a salt thereof, wherein:
Cy is independently monocyclic aryl or monocyclic heteroaryl;
R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR², —CONHR²⁵, and —NHCOR²⁵;
s is 0 to 4; and
each R²⁵ is independently selected from H, and optionally substituted (C₁-C₆)alkyl.

In some embodiments, when Cy is optionally substituted phenyl, then A is optionally substituted biphenyl of the formula:

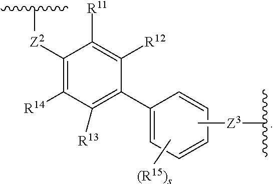

In some embodiments of formula (III)-(XIII), A is selected from:

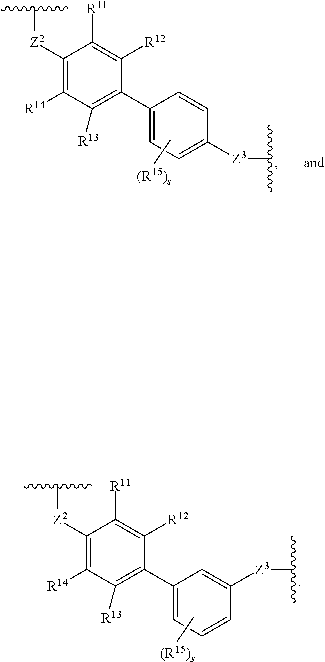

In some embodiments, when Cy is triazole, then A is selected from:

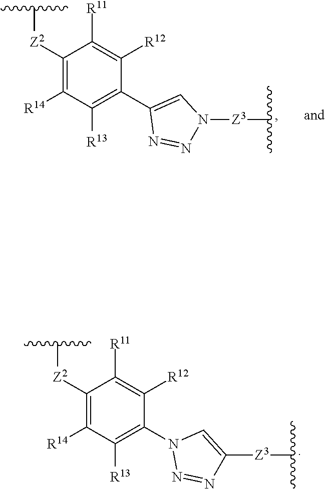

In some embodiments, at least one of R¹¹ to R¹⁵ is OH (e.g., at least two are OH).

In some embodiments, R¹¹ to R¹⁵ are each H.

### 4.2.3. Linking Moiety Z³

The linking moiety Z³ can be any convenient linking moiety that connects the linker L to the cyclic ring A. In some embodiments of formula (III)-(XIII), Z³ is has a backbone of 3 atoms or less.

In some embodiments of formula (III)-(XIII), Z³ is selected from a covalent bond, —O—NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (III)-(XIII), Z³ is a covalent bond connecting A to L.

In some embodiments of formula (III)-(XIII), Z³ is optionally substituted amido, urea or thiourea.

In some embodiments of formula (III)-(XIII), Z³ is

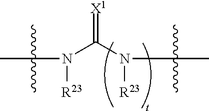

wherein:
X¹ is O or S;
t is 0 or 1; and
each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is urea or thiourea.

In some embodiments of formula (III)-(XIII), Z³ is —N(R²³)SO₂— or —SO₂N(R²³)—. In some embodiments of formula (III)-(XIII), Z³ is —NHSO₂— or —SO₂NH—.

In some embodiments of formula (III)-(XIII), Z³ is —N(R²³)CO— or —CON(R²³)—. In some embodiments of formula (III)-(XIII), Z³ is —NHCO— or —CONH—.

In some embodiments of formula (III)-(XIII), Z³ is —NHC(═X¹)NH—, wherein X¹ is O or S. In some embodiments, X¹ is O (i.e., Z³ is —NHC(═O)NH—). In some embodiments, X¹ is S.

In some embodiments of formula (III)-(XIII), Z³ is optionally substituted triazole. When Z³ is optionally substituted triazole, it can be synthetically derived from click chemistry conjugation of an azido containing precursor and an alkyne containing precursor of the compound.

In some embodiments, Z³ is selected in combination with cyclic group A and/or linking moiety Z¹ to provide desirable M6PR binding and internalization properties for X.

In some embodiments of formula (III)-(XIII), -A-Z³— is selected from:

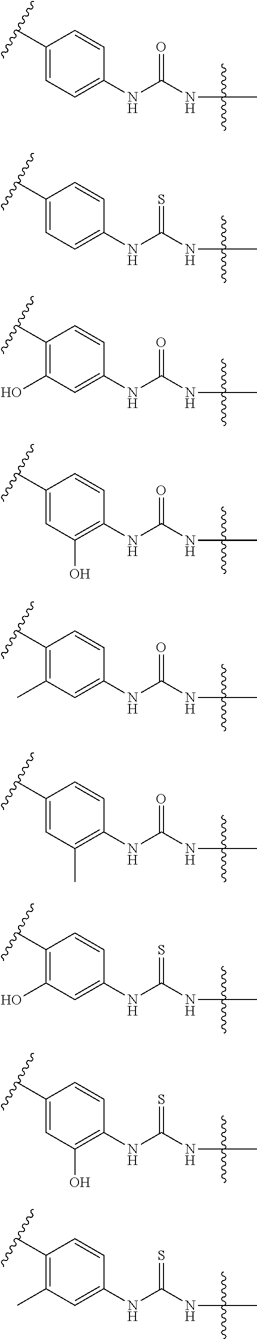

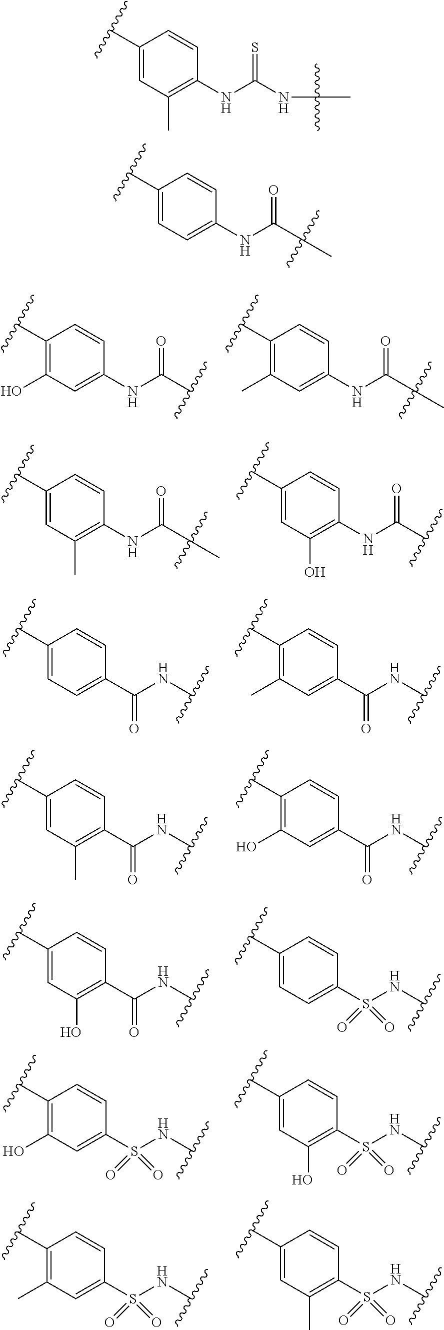

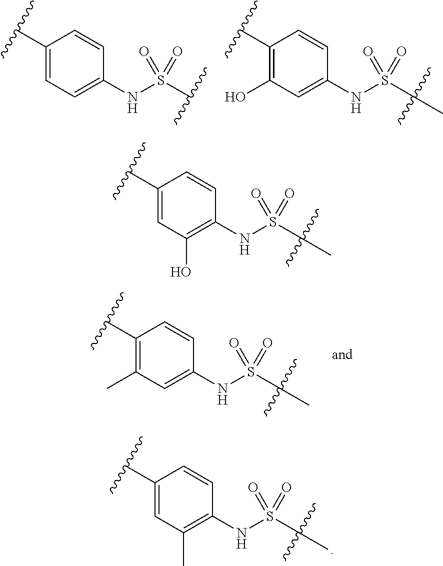 [SMILES: *c1ccc(NS(*)(=O)=O)cc1 ; *c1ccc(NS(*)(=O)=O)cc1O ; *c1ccc(NS(*)(=O)=O)c(O)c1 ; *c1ccc(NS(*)(=O)=O)cc1C ; *c1ccc(NS(*)(=O)=O)c(C)c1]

In some embodiments of formula (III)-(XIII), -A-Z³— is selected from:

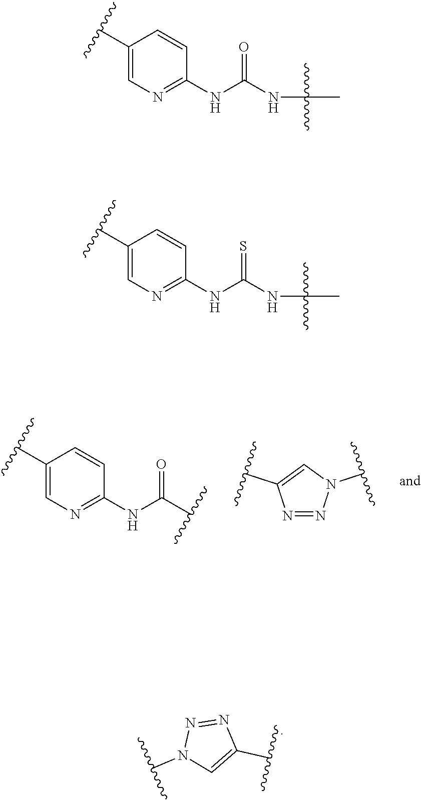 [SMILES: *NC(=O)Nc1ccc(*)cn1 ; *NC(=S)Nc1ccc(*)cn1 ; *C(=O)Nc1ccc(*)cn1 ; *c1cn(*)nn1 ; *c1cn(*)nn1]

In some embodiments of formula (III)-(XIII), -A-Z³— is selected from:

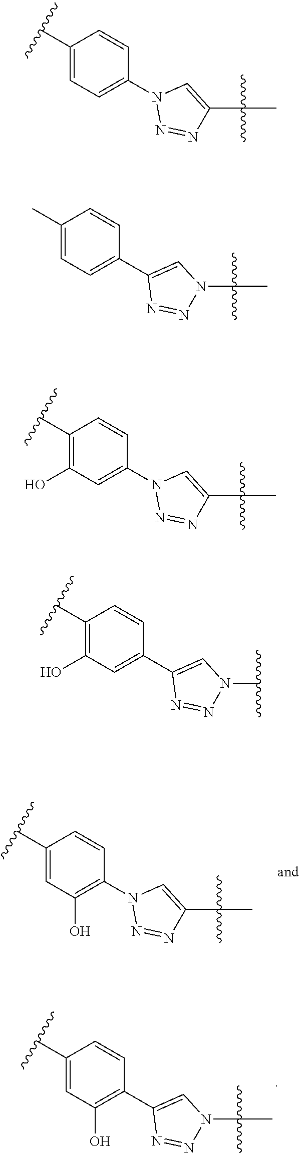 [SMILES: *c1ccc(-n2cc(*)nn2)cc1 ; *n1cc(-c2ccc(C)cc2)nn1 ; *c1cn(-c2ccc(*)c(O)c2)nn1 ; *c1ccc(-c2cn(*)nn2)cc1O ; *c1ccc(-n2cc(*)nn2)c(O)c1 ; *c1ccc(-c2cn(*)nn2)c(O)c1]

In some embodiments of formula (II)-(XIb), -A-Z³— is selected from:

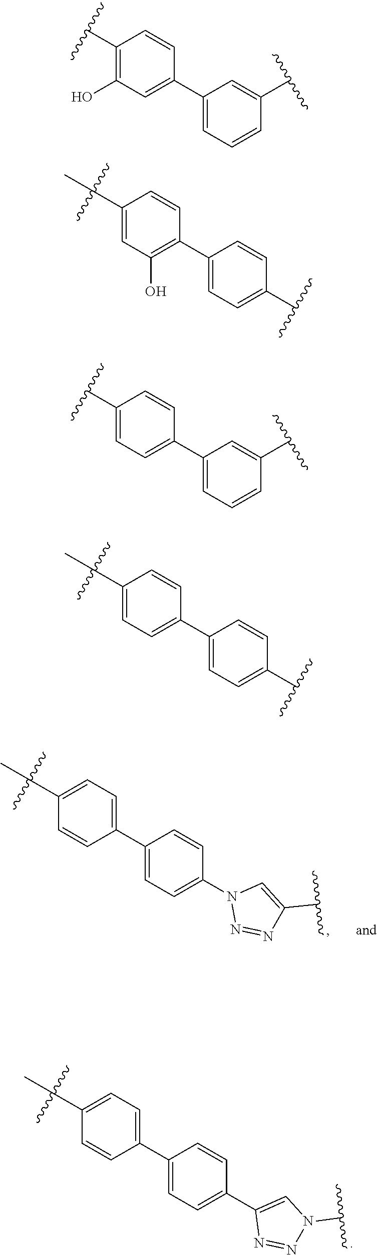 [SMILES: *c1cccc(-c2ccc(*)c(O)c2)c1 ; *c1ccc(-c2ccc(*)cc2O)cc1 ; *c1ccc(-c2cccc(*)c2)cc1 ; *c1ccc(-c2ccc(*)cc2)cc1 ; *c1ccc(-c2ccc(-n3cc(*)nn3)cc2)cc1 ; *c1ccc(-c2ccc(-c3cn(*)nn3)cc2)cc1]

In some embodiments of formula (III)-(XIII) -A-Z³— is selected from:

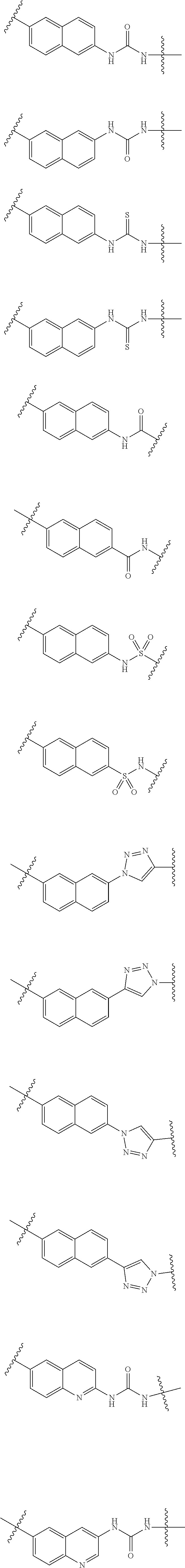

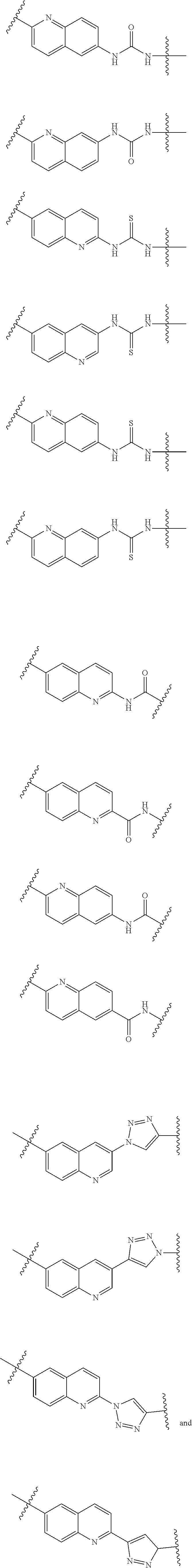

In some embodiments of formula (III)-(XIII), Z² is O.

In some embodiments of formula (III)-(XIII), Z² is S.

In some embodiments of formula (III)-(XIII), Z² is —NR²¹—.

In some embodiments of formula (III)-(XIII), Z² is —C(R²²)₂—, wherein each R²² is independently selected from H, halogen (e.g., F) and optionally substituted (C₁-C₆)alkyl. In some embodiments, Z² is —CH₂—. In some embodiments, Z² is —CHF—. In some embodiments, Z² is —CF₂—.

In some embodiments of formula (III)-(XIII), Z²-A-Z³— is

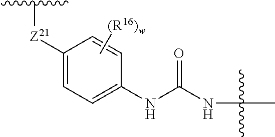

wherein:
Z²¹ is O, S, or —C(R²²)₂—;
R¹⁶ is OH or CH₃; and
w is 0 to 4 (e.g., w is 0, 1, or 2).

In some embodiments, Z²¹ is S or O. In some embodiments, Z²¹ is —CH₂—. In some embodiments, Z²¹ is —CHF—. In some embodiments, Z²¹ is —CF₂—. In some embodiments, R¹⁶ is OH and w is 1. In some embodiments, R¹⁶ is CH₃ and w is 1. In some embodiments, w is 0.

In some embodiments of formula (III)-(XIII), —Z²-A-Z³— is:

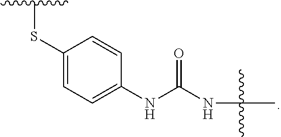 [SMILES: *NC(=O)Nc1ccc(S*)cc1]

In some embodiments of formula (III)-(XII), —Z²-A-Z³— is

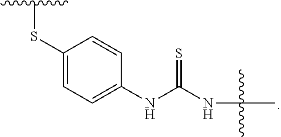 [SMILES: *NC(=S)Nc1ccc(S*)cc1]

In some embodiments of formula (III)-(XIII), —Z²-A-Z³— is

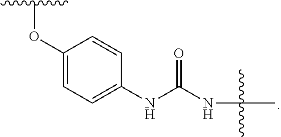 [SMILES: *NC(=O)Nc1ccc(O*)cc1]

In some embodiments of formula (III)-(XIII), —Z²-A-Z³— is

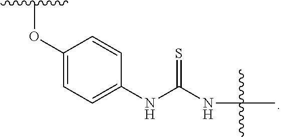 [SMILES: *NC(=S)Nc1ccc(O*)cc1]

In some embodiments of formula (III)-(XIII), —Z²-A-Z³— is

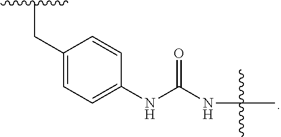 [SMILES: *Cc1ccc(NC(=O)N*)cc1]

In some embodiments of formula (III)-(XIII), —Z²-A-Z³— is

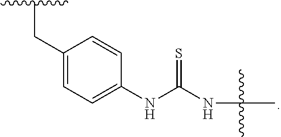 [SMILES: *Cc1ccc(NC(=S)N*)cc1]

### 4.2.4. Exemplary M6PR Ligands

Exemplary M6PR binding moieties, X, of formula (I)-(XIII) which can be utilized in the preparation of compounds and conjugates of this disclosure are shown in Table 1.

TABLE 1
Exemplary M6PR binding moieties, X

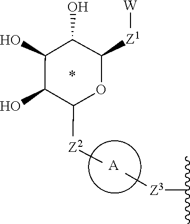

| # | W | Z¹ | Z² | * | A | Z³ |
|---|---|---|---|---|---|---|
| X1 | —P(═O)(OH)₂ | —CH₂CH₂— | —O— | alpha | 1,4-phenylene | —NHCO— |
| X2 | —P(═O)(OH)₂ | —CH₂CH₂— | —O— | alpha | 1,4-phenylene | —NHC(═S)NH— |
| X3 | —P(═O)(OH)₂ | —CH₂CH₂— | —O— | alpha | 1,4-phenylene | —NHC(═O)NH— |
| X4 | —P(═O)(OH)₂ | —CH₂CH₂— | —O— | alpha | 1,4-phenylene | —CH₂— |
| X5 | —P(═O)(OH)₂ | —CH₂CH₂— | —O— | alpha | 1,4-phenylene | —OCH₂— |
| X6 | —P(═O)(OH)₂ | —CH₂CH₂— | —O— | alpha | 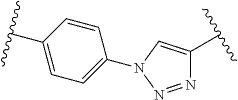 [SMILES: *c1ccc(-n2cc(*)nn2)cc1] | — |
| X7 | —P(═O)(OH)₂ | —CH₂CH₂— | —O— | alpha | 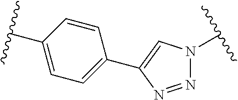 [SMILES: *c1ccc(-c2cn(*)nn2)cc1] | — |
| X8 | —P(═O)(OH)₂ | —CH₂CH₂— | —O— | alpha | 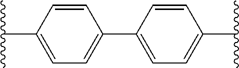 [SMILES: *c1ccc(-c2ccc(*)cc2)cc1] | — |
| X9 | —P(═O)(OH)₂ | —CH₂CH₂— | —O— | alpha | 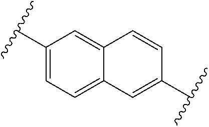 [SMILES: *c1ccc2cc(*)ccc2c1] | —NHCO— |
| X10 | —P(═O)(OH)₂ | —CH₂CH₂— | —O— | alpha | 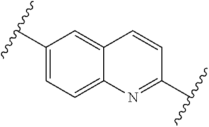 [SMILES: *c1ccc2nc(*)ccc2c1] | —NHCO— |
| X11 | —P(═O)(OH)₂ | —CH₂CH₂— | —S— | alpha | 1,4-phenylene | —NHC(═O)NH— |
| X12 | —P(═O)(OH)₂ | —CH₂CH2— | —O— | alpha | 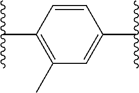 [SMILES: *c1ccc(*)c(C)c1] | —NHC(═O)NH— |
| X13 | —P(═O)(OH)₂ | —CH₂CH₂— | —O— | alpha | 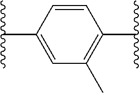 [SMILES: *c1ccc(*)c(C)c1] | —NHC(═O)NH— |
| X14 | —P(═O)(OH)₂ | —CH₂CH₂— | —O— | alpha | 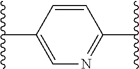 [SMILES: *c1ccc(*)nc1] | —NHC(═O)NH— |
| X15 | —P(═O)(OH)₂ | —CH₂CH₂— | —O— | alpha | 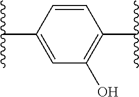 [SMILES: *c1ccc(*)c(O)c1] | —NHC(═O)NH— |
| X16 | —P(═O)(OH)₂ | —CH₂CH₂— | —O— | alpha | 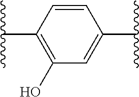 [SMILES: *c1ccc(*)c(O)c1] | —NHC(═O)NH— |
| X17 | —P(═O)(OH)₂ | —CH₂CF₂— | —O— | alpha | 1,4-phenylene | —NHCO— |
| X18 | —P(═O)(OH)₂ | —CH₂CF₂— | —O— | alpha | 1,4-phenylene | —NHC(═S)NH— |
| X19 | —COOH | —CH₂CH₂— | —O— | alpha | 1,4-phenylene | —NHC(═S)NH— |
| X20 | —COOH | —CH═CH— | —O— | alpha | 1,4-phenylene | —NHC(═O)NH— |
| X21 | —CH(COOH)₂ | —CH₂— | —O— | alpha | 1,4-phenylene | —NHCO— |
| X22 | —CH(COOH)₂ | —CH₂— | —O— | alpha | 1,4-phenylene | —NHC(═S)NH— |
| X23 | —CH(COOH)₂ | —CH₂— | —O— | alpha | 1,4-phenylene | —NHC(═O)NH— |
| X24 | —SO₃H | —CH₂CH₂— | —O— | alpha | 1,4-phenylene | —NHCO— |
| X25 | —SO₃H | —CH₂CH₂— | —O— | alpha | 1,4-phenylene | —NHC(═S)NH— |
| X26 | —P(═O)(OH)₂ | —CH₂CH₂— | —CH₂— | alpha | 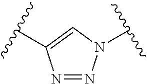 [SMILES: *c1cn(*)nn1] | — |
| X27 | —P(═O)(OH)₂ | —CH₂CH₂— | —CH₂— | alpha | 1,4-phenylene | —NHC(═O)NH— |
| X28 | —NHC(═O)CO₂H | —CH₂— | —O— | alpha | 1,4-phenylene | —NHC(═O)NH— |
| X29 | 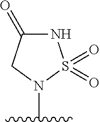 [SMILES: *N1CC(=O)NS1(=O)=O] | —CH₂— | —O— | alpha | 1,4-phenylene | —NHC(═O)NH— |
| X30 | —NHSO₂Me | —CH₂— | —O— | alpha | 1,4-phenylene | —NHC(═O)NH— |
| X31 | —NHSO₂NH₂ | —CH₂— | —O— | alpha | 1,4-phenylene | —NHC(═O)NH— |
| X32 | —NHC(═O)NHSO₂Me | —CH₂— | —O— | alpha | 1,4-phenylene | —NHC(═O)NH— |
| X33 | —NHSO₃H | —CH₂— | —O— | alpha | 1,4-phenylene | —NHC(═O)NH— |
| X34 | —P(═)(OH)₂ | —CH₂CH₂— | —O— | alpha | 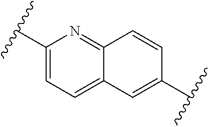 [SMILES: *c1ccc2nc(*)ccc2c1] | —NHC(═O)NH— |
| X35 | —P(═O)(OH)₂ | —CH₂CH₂— | —O— | alpha | 1,4-phenylene | —CONH— |
| X36 | —P(═O)(OH)₂ | —CH₂CH₂— | —O— | alpha | 1,4-phenylene | —NHSO₂— |
| X37 | —P(═O)(OH)₂ | —CH₂CH₂— | —O— | alpha | 1,4-phenylene | —SO₂NH₂ |
| X38 | —NHSO₂CF₃ | —CH₂— | —O— | alpha | 1,4-phenylene | —NHC(═O)NH— |
| X39 | —P(═O)(OH)₂ | —CH₂CH₂— | —CF₂— | alpha | 1,4-phenylene | —NHC(═O)NH— |
| X40 | —P(═O)(OH)₂ | —CH₂CH₂— | —CF₂— | alpha | 1,4-phenylene | —NHC(═S)NH— |
| X41 | —P(═O)(OH)₂ | —CH₂CH₂— | —CH₂— | alpha | 1,4-phenylene | —NHC(═S)NH— |
| X42 | —P(═O)(OH)₂ | —CH₂CH₂— | —S— | alpha | 1,4-phenylene | —NHC(═S)NH— |
| X43 | —P(═O)(OH)₂ | —CH₂CH₂— | —S— | alpha | 1,4-phenylene | —NHCO— |
| X44 | —P(═O)(OH)₂ | —CH₂CH₂— | —S— | alpha | 1,4-phenylene | —CONH— |
| X45 | —P(═O)(OH)₂ | —CH₂CH₂— | —S— | alpha | 1,4-phenylene | —NHSO₂— |
| X46 | —P(═O)(OH)₂ | —CH₂CH₂ — | —S— | alpha | 1,4-phenylene | —SO₂NH— |
| X47 | —P(═O)(OH)₂ | —CH₂CH₂— | —S— | alpha | 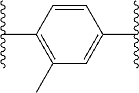 [SMILES: *c1ccc(*)c(C)c1] | —NHC(═O)NH— |
| X48 | —P(═O)(OH)₂ | —CH₂CH₂— | —S— | alpha | 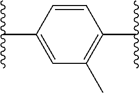 [SMILES: *c1ccc(*)c(C)c1] | —NHC(═O)NH— |
| X49 | —P(═O)(OH)₂ | —CH₂CH₂— | —S— | alpha | 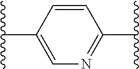 [SMILES: *c1ccc(*)nc1] | —NHC(═O)NH— |
| X50 | —P(═O)(OH)₂ | —CH₂CH₂— | —S— | alpha | 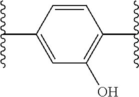 [SMILES: *c1ccc(*)c(O)c1] | —NHC(═O)NH— |
| X51 | —P(═O)(OH)₂ | —CH₂CH₂— | —S— | alpha | 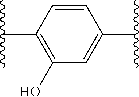 [SMILES: *c1ccc(*)c(O)c1] | —NHC(═O)NH— |

Beta configuration moieties

| | | | | | | |
|---|---|---|---|---|---|---|
| X1* | —P(═O)(OH)₂ | —CH₂CH₂— | —O— | beta | 1,4-phenylene | —NHCO— |
| X2* | —P(═O)(OH)₂ | —CH₂CH₂— | —O— | beta | 1,4-phenylene | —NHC(═S)NH— |
| X3* | —P(═O)(OH)₂ | —CH₂CH₂— | —O— | beta | 1,4-phenylene | —NHC(═O)NH— |
| X4* | —P(═O)(OH)₂ | —CH₂CH₂— | —O— | beta | 1,4-phenylene | —CH₂— |
| X5* | —P(═O)(OH)₂ | —CH₂CH₂— | —O— | beta | 1,4-phenylene | —OCH₂— |
| X6* | —P(═O)(OH)₂ | —CH₂CH₂— | —O— | beta | 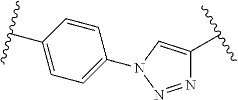 [SMILES: *c1ccc(-n2cc(*)nn2)cc1] | — |
| X7* | —P(═O)(OH)₂ | —CH₂CH₂— | —O— | beta | 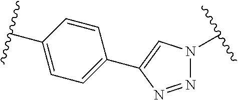 [SMILES: *c1ccc(-c2cn(*)nn2)cc1] | — |
| X8* | —P(═O)(OH)₂ | —CH₂CH₂— | —O— | beta | 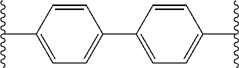 [SMILES: *c1ccc(-c2ccc(*)cc2)cc1] | — |
| X9* | —P(═O)(OH)₂ | —CH₂CH₂— | —O— | beta | 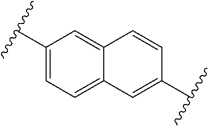 [SMILES: *c1ccc2cc(*)ccc2c1] | —NHCO— |
| X10* | —P(═O)(OH)₂ | —CH₂CH₂— | —O— | beta | 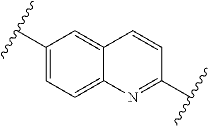 [SMILES: *c1ccc2nc(*)ccc2c1] | —NHCO— |
| X11* | —P(═O)(OH)₂ | —CH₂CH₂— | —S— | beta | 1,4-phenylene | —NHC(═O)NH— |
| X12* | —P(═O)(OH)₂ | —CH₂CH₂— | —O— | beta | 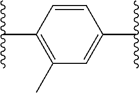 [SMILES: *c1ccc(*)c(C)c1] | —NHC(═O)NH— |
| X13* | —P(═O)(OH)₂ | —CH₂CH₂— | —O— | beta | 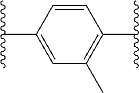 [SMILES: *c1ccc(*)c(C)c1] | —NHC(═O)NH— |
| X14* | —P(═O)(OH)₂ | —CH₂CH₂— | —O— | beta | 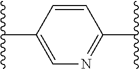 [SMILES: *c1ccc(*)nc1] | —NHC(═O)NH— |
| X15* | —P(═O)(OH)₂ | —CH₂CH₂— | —O— | beta | 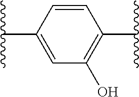 [SMILES: *c1ccc(*)c(O)c1] | —NHC(═O)NH— |
| X16* | —P(═O)(OH)₂ | —CH₂CH₂— | —O— | beta | 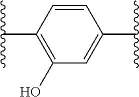 [SMILES: *c1ccc(*)c(O)c1] | —NHC(═O)NH— |
| X17* | —P(═O)(OH)₂ | —CH₂CF₂— | —O— | beta | 1,4-phenylene | —NHCO— |
| X18* | —P(═O)(OH)₂ | —CH₂CF₂— | —O— | beta | 1,4-phenylene | —NHC(═S)NH— |
| X19* | —COOH | —CH₂CH₂— | —O— | beta | 1,4-phenylene | —NHC(═S)NH— |
| X20* | —COOH | —CH═CH— | —O— | beta | 1,4-phenylene | —NHC(═O)NH— |
| X21* | —CH(COOH)₂ | —CH₂— | —O— | beta | 1,4-phenylene | —NHCO— |
| X22* | —CH(COOH)₂ | —CH₂— | —O— | beta | 1,4-phenylene | —NHC(═S)NH— |
| X23* | —CH(COOH)₂ | —CH₂— | —O— | beta | 1,4-phenylene | —NHC(═O)NH— |
| X24* | —SO₃H | —CH₂CH₂— | —O— | beta | 1,4-phenylene | —NHCO— |
| X25* | —SO₃H | —CH₂CH_{2 —} | —O— | beta | 1,4-phenylene | —NHC(═S)NH— |
| X26* | —P(═O)(OH)₂ | —CH₂CH₂— | —CH₂— | beta | 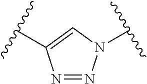 [SMILES: *c1cn(*)nn1] | — |
| X27* | —P(═O)(OH)₂ | —CH₂CH₂— | —CH₂— | beta | 1,4-phenylene | —NHC(═O)NH— |
| X28* | —NHC(═O)CO₂H | —CH₂— | —O— | beta | 1,4-phenylene | —NHC(═O)NH— |
| X29* | 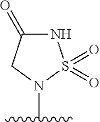 [SMILES: *N1CC(=O)NS1(=O)=O] | —CH₂— | —O— | beta | 1,4-phenylene | —NHC(═O)NH— |
| X30* | —NHSO₂Me | —CH₂— | —O— | beta | 1,4-phenylene | —NHC(═O)NH— |
| X31* | —NHSO₂NH₂ | —CH₂— | —O— | beta | 1,4-phenylene | —NHC(═O)NH— |
| X32* | —NHC(═O)NHSO₂Me | —CH₂— | —O— | beta | 1,4-phenylene | —NHC(═O)NH— |
| X33* | —NHSO₃H | —CH₂— | —O— | beta | 1,4-phenylene | —NHC(═O)NH— |
| X34* | —P(═O)(OH)₂ | —CH₂CH₂— | —O— | beta | 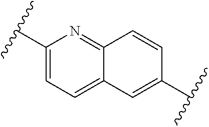 [SMILES: *c1ccc2nc(*)ccc2c1] | —NHC(═O)NH— |
| X35* | —P(═O)(OH)₂ | —CH₂CH₂— | —O— | beta | 1,4-phenylene | —CONH— |
| X36* | —P(═O)(OH)₂ | —CH₂CH₂— | —O— | beta | 1,4-phenylene | —NHSO₂— |
| X37* | —P(═O)(OH)₂ | —CH₂CH₂— | —O— | beta | 1,4-phenylene | —SO₂NH— |
| X38* | —NHSO₂CF₃ | —CH₂— | —O— | beta | 1,4-phenylene | —NHC(═O)NH— |
| X39* | —P(═O)(OH)₂ | —CH₂CH₂— | —CF₂— | beta | 1,4-phenylene | —NHC(═O)NH— |
| X40* | —P(═O)(OH)₂ | —CH₂CH₂— | —CF₂— | beta | 1,4-phenylene | —NHC(═S)NH— |
| X41* | —P(═O)(OH)₂ | —CH₂CH₂— | —CH₂ — | beta | 1,4-phenylene | —NHC(═S)NH— |
| X42* | —P(═O)(OH)₂ | —CH₂CH₂— | —S— | beta | 1,4-phenylene | —NHC(═S)NH— |
| X43* | —P(═O)(OH)₂ | —CH₂CH₂— | —S— | beta | 1,4-phenylene | —NHCO— |
| X44* | —P(═O)(OH)₂ | —CH₂CH₂— | —S— | beta | 1,4-phenylene | —CONH— |
| X45* | —P(═O)(OH)₂ | —CH₂CH₂— | —S— | beta | 1,4-phenylene | —NHSO₂— |
| X46* | —P(═O)(OH)₂ | —CH₂CH₂— | —S— | beta | 1,4-phenylene | —SO₂NH— |
| X47* | —P(═O)(OH)₂ | —CH₂CH₂— | —S— | beta | 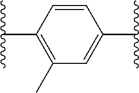 [SMILES: *c1ccc(*)c(C)c1] | —NHC(═O)NH— |
| X48* | —P(═O)(OH)₂ | —CH₂CH₂— | —S— | beta | 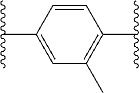 [SMILES: *c1ccc(*)c(C)c1] | —NHC(═O)NH— |
| X49* | —P(═O)(OH)₂ | —CH₂CH₂— | —S— | beta | 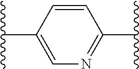 [SMILES: *c1ccc(*)nc1] | —NHC(═O)NH— |
| X50* | —P(═O)(OH)₂ | —CH₂CH₂— | —S— | beta | 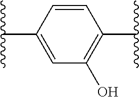 [SMILES: *c1ccc(*)c(O)c1] | —NHC(═O)NH— |
| X51* | —P(═O)(OH)₂ | —CH₂CH₂— | —S— | beta | 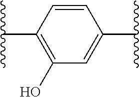 [SMILES: *c1ccc(*)c(O)c1] | —NHC(═O)NH— |

alpha refers to the following configuration: 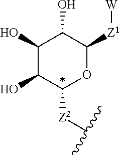
*beta refers to the following configuration:

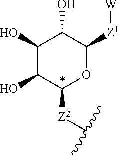

Exemplary synthons or synthetic precursors which can be utilized in the preparation of compounds of this disclosure to incorporate a desired M6PR binding moiety of interest are shown in Table 2. It is understood that alternative synthons, including homologs and analogs of the ones shown in Table 2 are possible depending on the M6PR binding moiety and linker that is selected. It is understood that the synthons of Table 2 can include structural precursors of linking moiety Z³, and a structural element that becomes part of the linker (L) in the compounds and conjugates of this disclosure. It is understood that based on the exemplary synthetic precursors of Table 2, synthons corresponding to any of the M6PR binding moieties of Table 1 can be utilized to prepare compounds of this disclosure.

TABLE 2
Exemplary Synthetic precursors for M6PR binding moieties

| Exemplary M6PR binding moiety (X) | | Exemplary Synthetic precursor(s) |
|---|---|---|
| # | Structure | Structure |
| X1 | 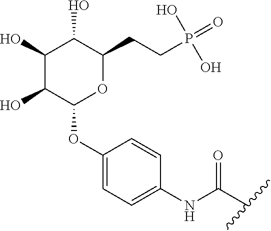 [SMILES: *C(=O)Nc1ccc(O[C@H]2O[C@H](CCP(=O)(O)O)[C@@H](O)[C@H](O)[C@@H]2O)cc1] | 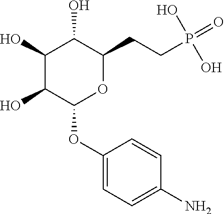 [SMILES: Nc1ccc(O[C@H]2O[C@H](CCP(=O)(O)O)[C@@H](O)[C@H](O)[C@@H]2O)cc1] |
| | | 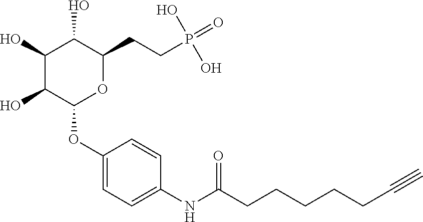 [SMILES: C#CCCCCCC(=O)Nc1ccc(O[C@H]2O[C@H](CCP(=O)(O)O)[C@@H](O)[C@H](O)[C@@H]2O)cc1] |
| X2 | 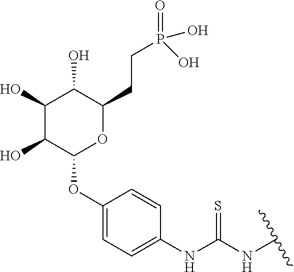 [SMILES: *NC(=S)Nc1ccc(O[C@H]2O[C@H](CCP(=O)(O)O)[C@@H](O)[C@H](O)[C@@H]2O)cc1] | 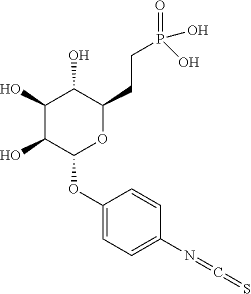 [SMILES: O=P(O)(O)CC[C@H]1O[C@H](Oc2ccc(N=C=S)cc2)[C@@H](O)[C@@H](O)[C@@H]1O] |
| | | 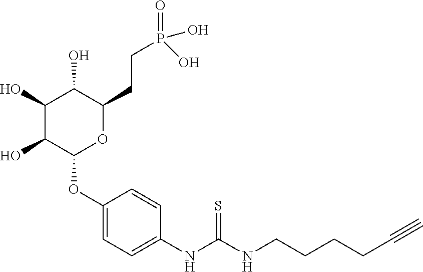 [SMILES: C#CCCCCNC(=S)Nc1ccc(O[C@H]2O[C@H](CCP(=O)(O)O)[C@@H](O)[C@H](O)[C@@H]2O)cc1] |
| X3 | 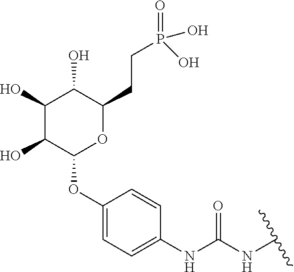 [SMILES: *NC(=O)Nc1ccc(O[C@H]2O[C@H](CCP(=O)(O)O)[C@@H](O)[C@H](O)[C@@H]2O)cc1] | 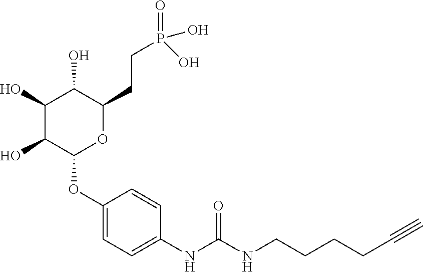 [SMILES: C#CCCCCNC(=O)Nc1ccc(O[C@H]2O[C@H](CCP(=O)(O)O)[C@@H](O)[C@H](O)[C@@H]2O)cc1] |
| X4 | 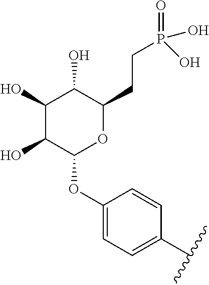 [SMILES: *c1ccc(O[C@H]2O[C@H](CCP(=O)(O)O)[C@@H](O)[C@H](O)[C@@H]2O)cc1] | 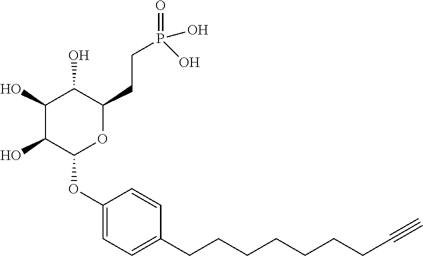 [SMILES: C#CCCCCCCCc1ccc(O[C@H]2O[C@H](CCP(=O)(O)O)[C@@H](O)[C@H](O)[C@@H]2O)cc1] |
| | | 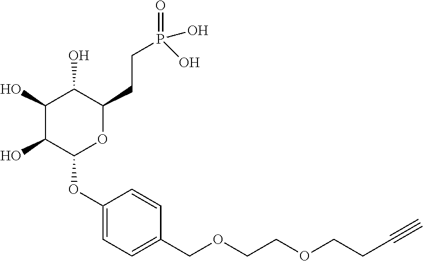 [SMILES: C#CCCOCCOCc1ccc(O[C@H]2O[C@H](CCP(=O)(O)O)[C@@H](O)[C@H](O)[C@@H]2O)cc1] |
| X5 | 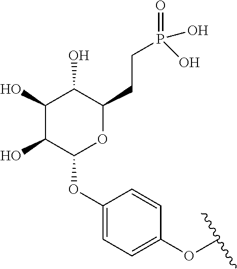 [SMILES: *Oc1ccc(O[C@H]2O[C@H](CCP(=O)(O)O)[C@@H](O)[C@H](O)[C@@H]2O)cc1] | 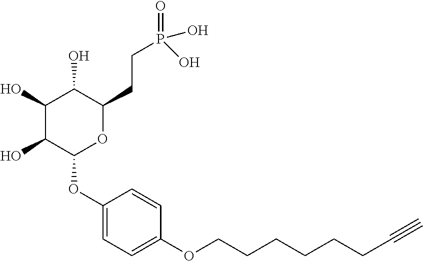 [SMILES: C#CCCCCCCOc1ccc(O[C@H]2O[C@H](CCP(=O)(O)O)[C@@H](O)[C@H](O)[C@@H]2O)cc1] |
| X6 | 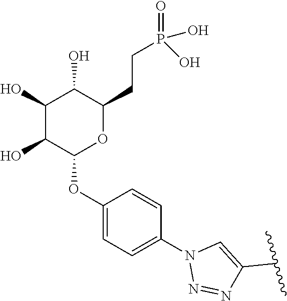 [SMILES: *c1cn(-c2ccc(O[C@H]3O[C@H](CCP(=O)(O)O)[C@@H](O)[C@H](O)[C@@H]3O)cc2)nn1] | 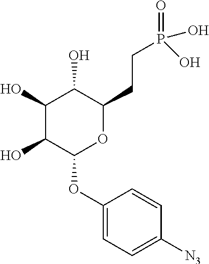 [SMILES: [N-]=[N+]=Nc1ccc(O[C@H]2O[C@H](CCP(=O)(O)O)[C@@H](O)[C@H](O)[C@@H]2O)cc1] |
| X7 | 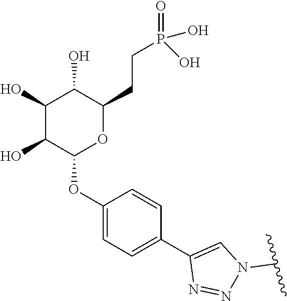 [SMILES: *n1cc(-c2ccc(O[C@H]3O[C@H](CCP(=O)(O)O)[C@@H](O)[C@H](O)[C@@H]3O)cc2)nn1] | 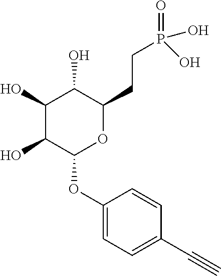 [SMILES: C#Cc1ccc(O[C@H]2O[C@H](CCP(=O)(O)O)[C@@H](O)[C@H](O)[C@@H]2O)cc1] |
| X8 | 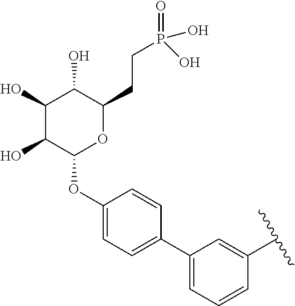 [SMILES: *c1cccc(-c2ccc(O[C@H]3O[C@H](CCP(=O)(O)O)[C@@H](O)[C@H](O)[C@@H]3O)cc2)c1] | 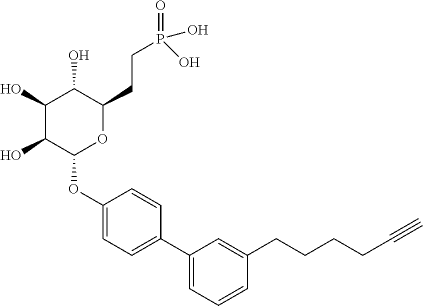 [SMILES: C#CCCCCc1cccc(-c2ccc(O[C@H]3O[C@H](CCP(=O)(O)O)[C@@H](O)[C@H](O)[C@@H]3O)cc2)c1] |
| X9 | 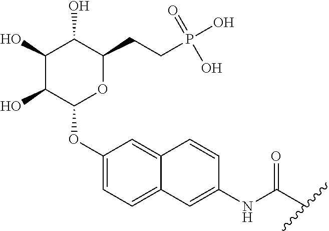 [SMILES: *C(=O)Nc1ccc2cc(O[C@H]3O[C@H](CCP(=O)(O)O)[C@@H](O)[C@H](O)[C@@H]3O)ccc2c1] | 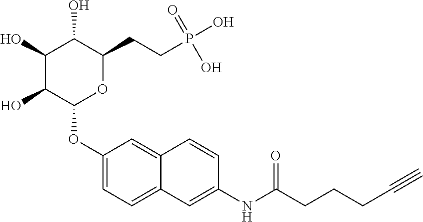 [SMILES: C#CCCCC(=O)Nc1ccc2cc(O[C@H]3O[C@H](CCP(=O)(O)O)[C@@H](O)[C@H](O)[C@@H]3O)ccc2c1] |
| X10 | 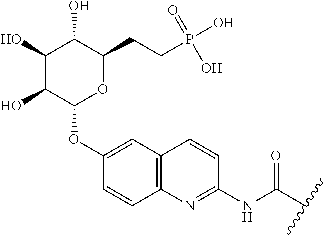 [SMILES: *C(=O)Nc1ccc2cc(O[C@H]3O[C@H](CCP(=O)(O)O)[C@@H](O)[C@H](O)[C@@H]3O)ccc2n1] | 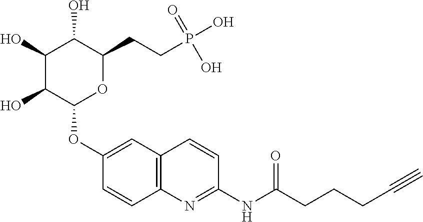 [SMILES: C#CCCCC(=O)Nc1ccc2cc(O[C@H]3O[C@H](CCP(=O)(O)O)[C@@H](O)[C@H](O)[C@@H]3O)ccc2n1] |
| X11 | 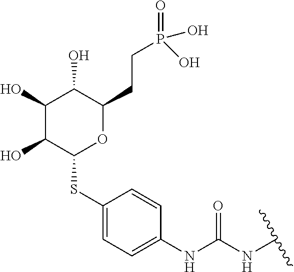 [SMILES: *NC(=O)Nc1ccc(S[C@H]2O[C@H](CCP(=O)(O)O)[C@@H](O)[C@H](O)[C@@H]2O)cc1] | 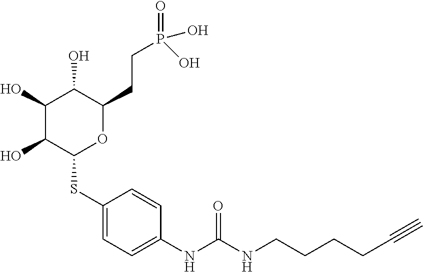 [SMILES: C#CCCCCNC(=O)Nc1ccc(S[C@H]2O[C@H](CCP(=O)(O)O)[C@@H](O)[C@H](O)[C@@H]2O)cc1] |
| X11* | 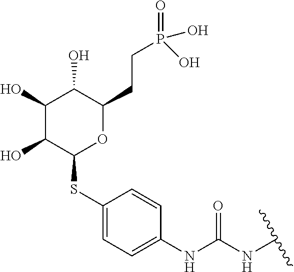 [SMILES: *NC(=O)Nc1ccc(S[C@@H]2O[C@H](CCP(=O)(O)O)[C@@H](O)[C@H](O)[C@@H]2O)cc1] | 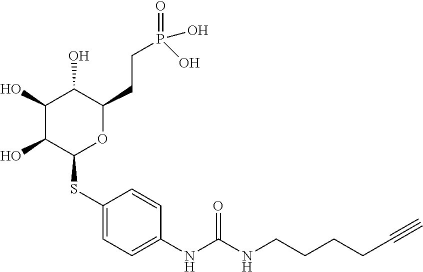 [SMILES: C#CCCCCNC(=O)Nc1ccc(S[C@@H]2O[C@H](CCP(=O)(O)O)[C@@H](O)[C@H](O)[C@@H]2O)cc1] |
| X12 | 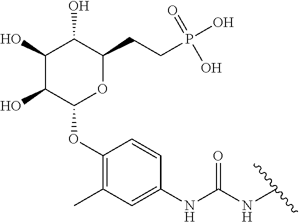 [SMILES: *NC(=O)Nc1ccc(O[C@H]2O[C@H](CCP(=O)(O)O)[C@@H](O)[C@H](O)[C@@H]2O)c(C)c1] | 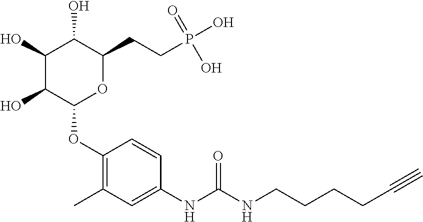 [SMILES: C#CCCCCNC(=O)Nc1ccc(O[C@H]2O[C@H](CCP(=O)(O)O)[C@@H](O)[C@H](O)[C@@H]2O)c(C)c1] |
| X13 | 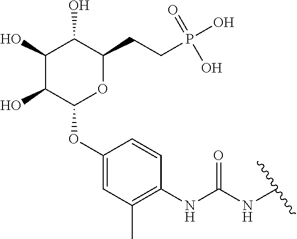 [SMILES: *NC(=O)Nc1ccc(O[C@H]2O[C@H](CCP(=O)(O)O)[C@@H](O)[C@H](O)[C@@H]2O)cc1C] | 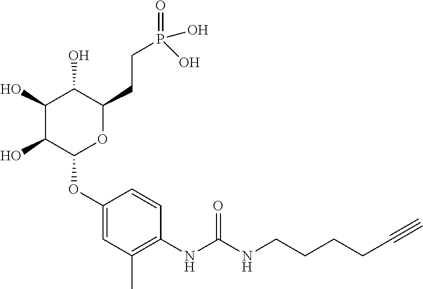 [SMILES: C#CCCCCNC(=O)Nc1ccc(O[C@H]2O[C@H](CCP(=O)(O)O)[C@@H](O)[C@H](O)[C@@H]2O)cc1C] |
| X14 | 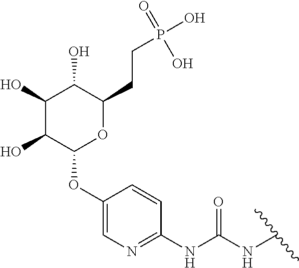 [SMILES: *NC(=O)Nc1ccc(O[C@H]2O[C@H](CCP(=O)(O)O)[C@@H](O)[C@H](O)[C@@H]2O)cn1] | 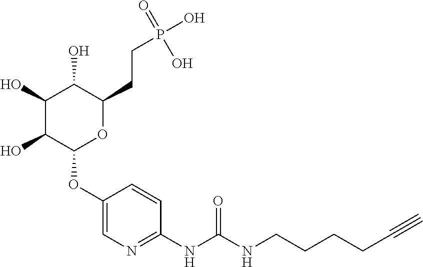 [SMILES: C#CCCCCNC(=O)Nc1ccc(O[C@H]2O[C@H](CCP(=O)(O)O)[C@@H](O)[C@H](O)[C@@H]2O)cn1] |
| X15 | 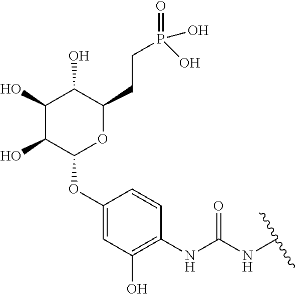 [SMILES: *NC(=O)Nc1ccc(O[C@H]2O[C@H](CCP(=O)(O)O)[C@@H](O)[C@H](O)[C@@H]2O)cc1O] | 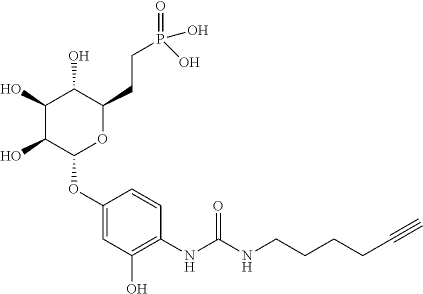 [SMILES: C#CCCCCNC(=O)Nc1ccc(O[C@H]2O[C@H](CCP(=O)(O)O)[C@@H](O)[C@H](O)[C@@H]2O)cc1O] |
| X16 | 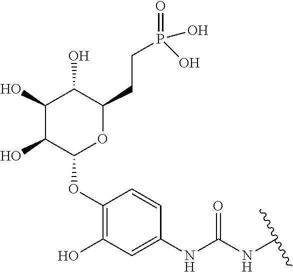 [SMILES: *NC(=O)Nc1ccc(O[C@H]2O[C@H](CCP(=O)(O)O)[C@@H](O)[C@H](O)[C@@H]2O)c(O)c1] | 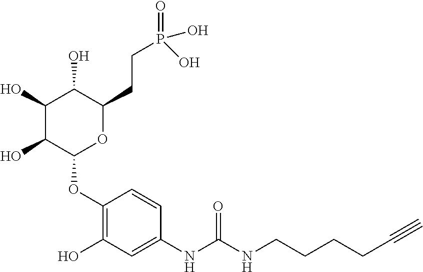 [SMILES: C#CCCCCNC(=O)Nc1ccc(O[C@H]2O[C@H](CCP(=O)(O)O)[C@@H](O)[C@H](O)[C@@H]2O)c(O)c1] |
| X17 | 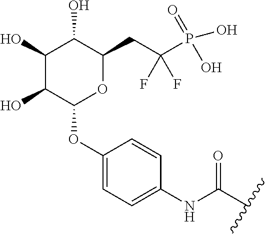 [SMILES: *C(=O)Nc1ccc(O[C@H]2O[C@H](CC(F)(F)P(=O)(O)O)[C@@H](O)[C@H](O)[C@@H]2O)cc1] | 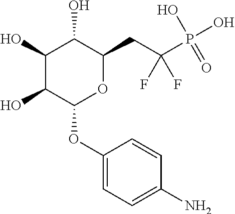 [SMILES: Nc1ccc(O[C@H]2O[C@H](CC(F)(F)P(=O)(O)O)[C@@H](O)[C@H](O)[C@@H]2O)cc1] |
| X18 | 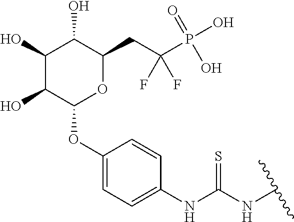 [SMILES: *NC(=S)Nc1ccc(O[C@H]2O[C@H](CC(F)(F)P(=O)(O)O)[C@@H](O)[C@H](O)[C@@H]2O)cc1] | 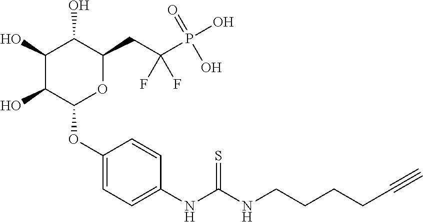 [SMILES: C#CCCCCNC(=S)Nc1ccc(O[C@H]2O[C@H](CC(F)(F)P(=O)(O)O)[C@@H](O)[C@H](O)[C@@H]2O)cc1] |
| X19 | 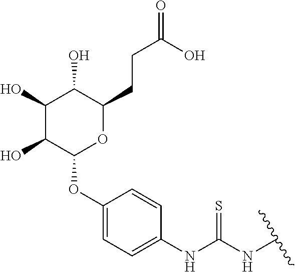 [SMILES: *NC(=S)Nc1ccc(O[C@H]2O[C@H](CCC(=O)O)[C@@H](O)[C@H](O)[C@@H]2O)cc1] | 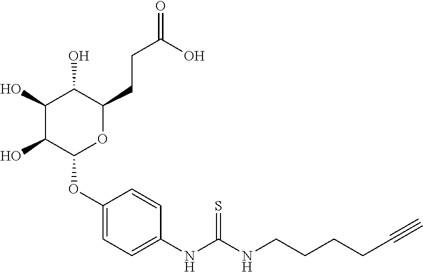 [SMILES: C#CCCCCNC(=S)Nc1ccc(O[C@H]2O[C@H](CCC(=O)O)[C@@H](O)[C@H](O)[C@@H]2O)cc1] |
| X20 | 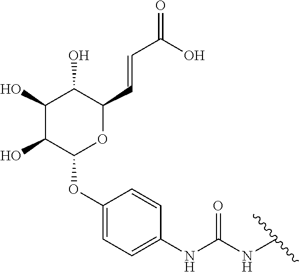 [SMILES: *NC(=O)Nc1ccc(O[C@H]2O[C@H](/C=C/C(=O)O)[C@@H](O)[C@H](O)[C@@H]2O)cc1] | 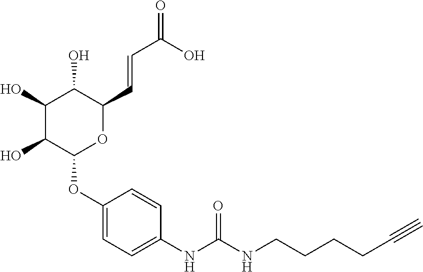 [SMILES: C#CCCCCNC(=O)Nc1ccc(O[C@H]2O[C@H](/C=C/C(=O)O)[C@@H](O)[C@H](O)[C@@H]2O)cc1] |
| X21 | 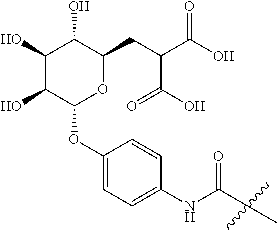 [SMILES: *C(=O)Nc1ccc(O[C@H]2O[C@H](CC(C(=O)O)C(=O)O)[C@@H](O)[C@H](O)[C@@H]2O)cc1] | 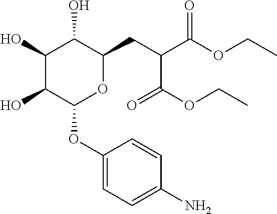 [SMILES: CCOC(=O)C(C[C@H]1O[C@H](Oc2ccc(N)cc2)[C@@H](O)[C@@H](O)[C@@H]1O)C(=O)OCC] |
| X22 | 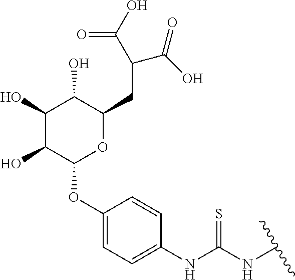 [SMILES: *NC(=S)Nc1ccc(O[C@H]2O[C@H](CC(C(=O)O)C(=O)O)[C@@H](O)[C@H](O)[C@@H]2O)cc1] | 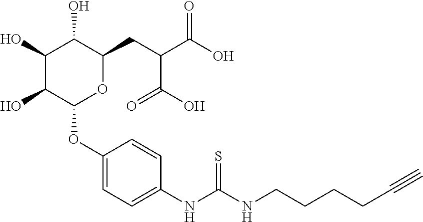 [SMILES: C#CCCCCNC(=S)Nc1ccc(O[C@H]2O[C@H](CC(C(=O)O)C(=O)O)[C@@H](O)[C@H](O)[C@@H]2O)cc1] |
| X23 | 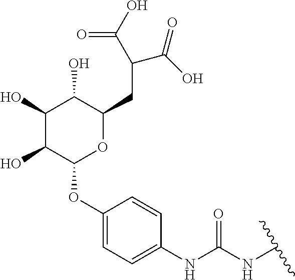 [SMILES: *NC(=O)Nc1ccc(O[C@H]2O[C@H](CC(C(=O)O)C(=O)O)[C@@H](O)[C@H](O)[C@@H]2O)cc1] | 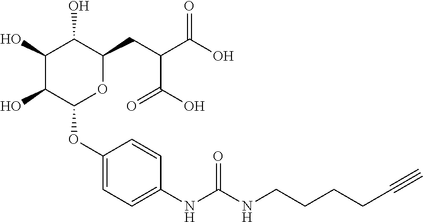 [SMILES: C#CCCCCNC(=O)Nc1ccc(O[C@H]2O[C@H](CC(C(=O)O)C(=O)O)[C@@H](O)[C@H](O)[C@@H]2O)cc1] |
| X24 | 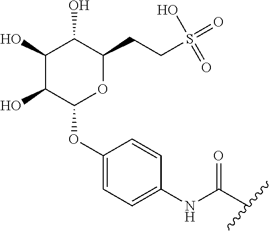 [SMILES: *C(=O)Nc1ccc(O[C@H]2O[C@H](CCS(=O)(=O)O)[C@@H](O)[C@H](O)[C@@H]2O)cc1] | 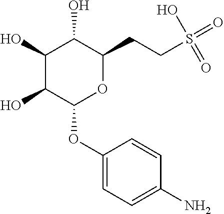 [SMILES: Nc1ccc(O[C@H]2O[C@H](CCS(=O)(=O)O)[C@@H](O)[C@H](O)[C@@H]2O)cc1] |
| X25 | 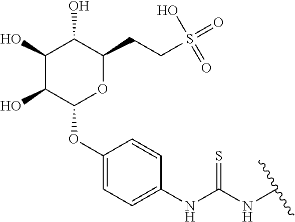 [SMILES: *NC(=S)Nc1ccc(O[C@H]2O[C@H](CCS(=O)(=O)O)[C@@H](O)[C@H](O)[C@@H]2O)cc1] | 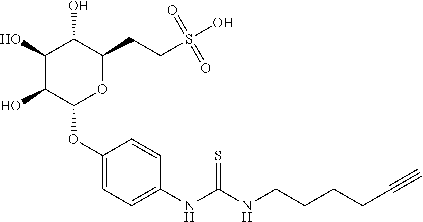 [SMILES: C#CCCCCNC(=S)Nc1ccc(O[C@H]2O[C@H](CCS(=O)(=O)O)[C@@H](O)[C@H](O)[C@@H]2O)cc1] |
| X26 | 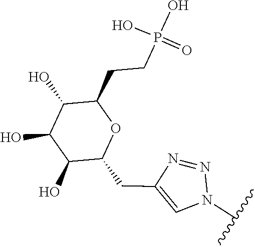 [SMILES: *n1cc(C[C@H]2O[C@H](CCP(=O)(O)O)[C@@H](O)[C@H](O)[C@@H]2O)nn1] | 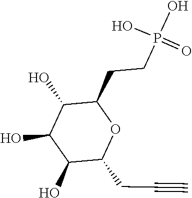 [SMILES: C#CC[C@H]1O[C@H](CCP(=O)(O)O)[C@@H](O)[C@H](O)[C@@H]1O] |
| X27 | 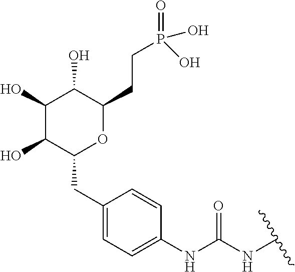 [SMILES: *NC(=O)Nc1ccc(C[C@H]2O[C@H](CCP(=O)(O)O)[C@@H](O)[C@H](O)[C@@H]2O)cc1] | 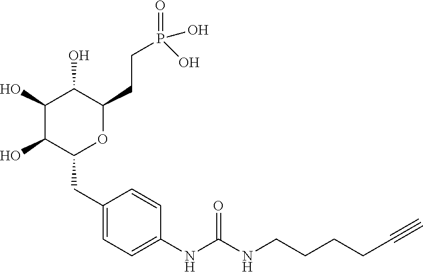 [SMILES: C#CCCCCNC(=O)Nc1ccc(C[C@H]2O[C@H](CCP(=O)(O)O)[C@@H](O)[C@H](O)[C@@H]2O)cc1] |
| X28 | 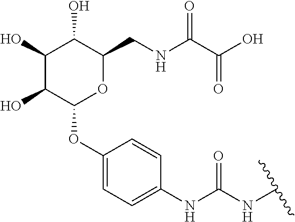 [SMILES: *NC(=O)Nc1ccc(O[C@H]2O[C@H](CNC(=O)C(=O)O)[C@@H](O)[C@H](O)[C@@H]2O)cc1] | 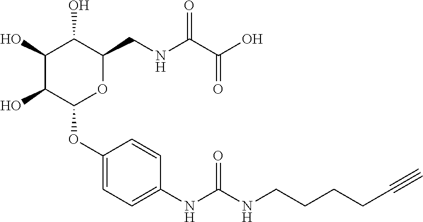 [SMILES: C#CCCCCNC(=O)Nc1ccc(O[C@H]2O[C@H](CNC(=O)C(=O)O)[C@@H](O)[C@H](O)[C@@H]2O)cc1] |
| X29 | 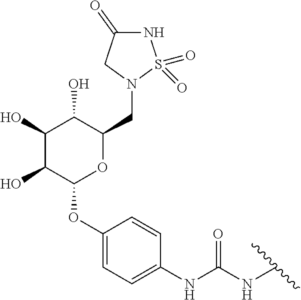 [SMILES: *NC(=O)Nc1ccc(O[C@H]2O[C@H](CN3CC(=O)NS3(=O)=O)[C@@H](O)[C@H](O)[C@@H]2O)cc1] | 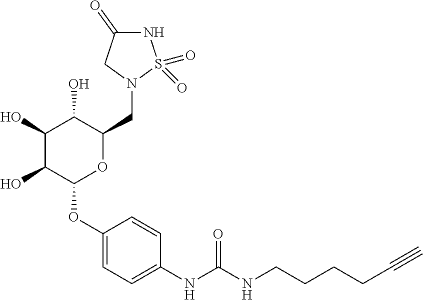 [SMILES: C#CCCCCNC(=O)Nc1ccc(O[C@H]2O[C@H](CN3CC(=O)NS3(=O)=O)[C@@H](O)[C@H](O)[C@@H]2O)cc1] |
| X30 | 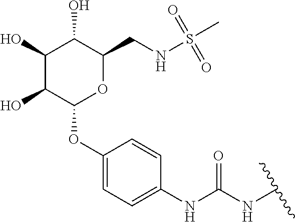 [SMILES: *NC(=O)Nc1ccc(O[C@H]2O[C@H](CNS(C)(=O)=O)[C@@H](O)[C@H](O)[C@@H]2O)cc1] | 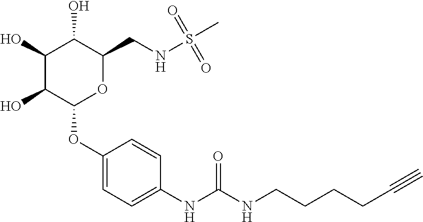 [SMILES: C#CCCCCNC(=O)Nc1ccc(O[C@H]2O[C@H](CNS(C)(=O)=O)[C@@H](O)[C@H](O)[C@@H]2O)cc1] |
| X31 | 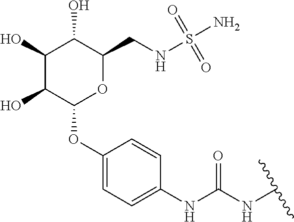 [SMILES: *NC(=O)Nc1ccc(O[C@H]2O[C@H](CNS(N)(=O)=O)[C@@H](O)[C@H](O)[C@@H]2O)cc1] | 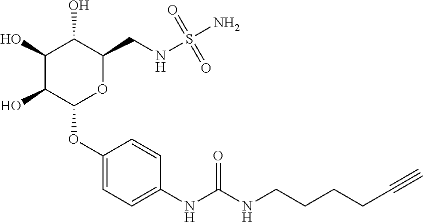 [SMILES: C#CCCCCNC(=O)Nc1ccc(O[C@H]2O[C@H](CNS(N)(=O)=O)[C@@H](O)[C@H](O)[C@@H]2O)cc1] |
| X32 | 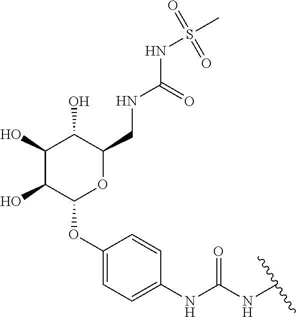 [SMILES: *NC(=O)Nc1ccc(O[C@H]2O[C@H](CNC(=O)NS(C)(=O)=O)[C@@H](O)[C@H](O)[C@@H]2O)cc1] | 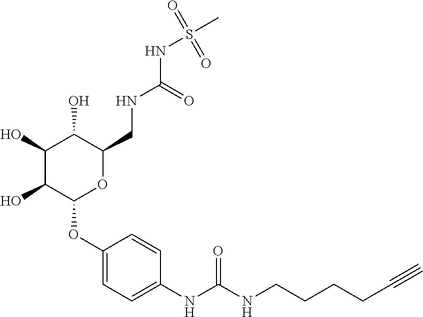 [SMILES: C#CCCCCNC(=O)Nc1ccc(O[C@H]2O[C@H](CNC(=O)NS(C)(=O)=O)[C@@H](O)[C@H](O)[C@@H]2O)cc1] |
| X33 | 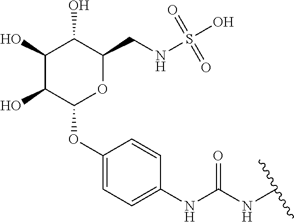 [SMILES: *NC(=O)Nc1ccc(O[C@H]2O[C@H](CNS(=O)(=O)O)[C@@H](O)[C@H](O)[C@@H]2O)cc1] | 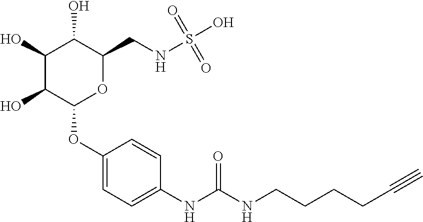 [SMILES: C#CCCCCNC(=O)Nc1ccc(O[C@H]2O[C@H](CNS(=O)(=O)O)[C@@H](O)[C@H](O)[C@@H]2O)cc1] |
| X34 | 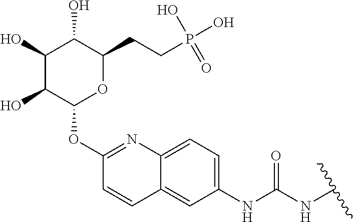 [SMILES: *NC(=O)Nc1ccc2nc(O[C@H]3O[C@H](CCP(=O)(O)O)[C@@H](O)[C@H](O)[C@@H]3O)ccc2c1] | 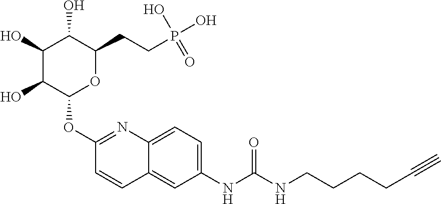 [SMILES: C#CCCCCNC(=O)Nc1ccc2nc(O[C@H]3O[C@H](CCP(=O)(O)O)[C@@H](O)[C@H](O)[C@@H]3O)ccc2c1] |
| X35 | 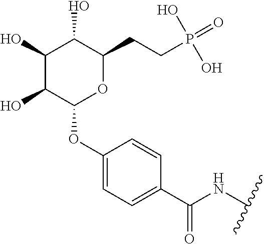 [SMILES: *NC(=O)c1ccc(O[C@H]2O[C@H](CCP(=O)(O)O)[C@@H](O)[C@H](O)[C@@H]2O)cc1] | 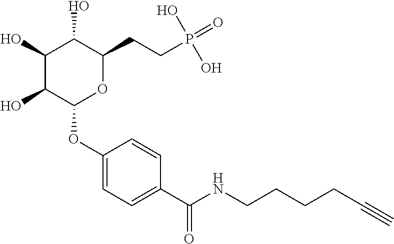 [SMILES: C#CCCCCNC(=O)c1ccc(O[C@H]2O[C@H](CCP(=O)(O)O)[C@@H](O)[C@H](O)[C@@H]2O)cc1] |
| X36 | 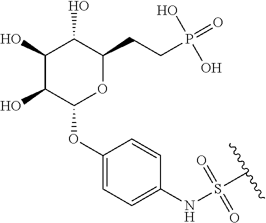 [SMILES: *S(=O)(=O)Nc1ccc(O[C@H]2O[C@H](CCP(=O)(O)O)[C@@H](O)[C@H](O)[C@@H]2O)cc1] | 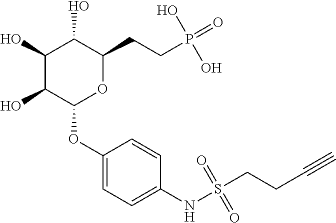 [SMILES: C#CCCS(=O)(=O)Nc1ccc(O[C@H]2O[C@H](CCP(=O)(O)O)[C@@H](O)[C@H](O)[C@@H]2O)cc1] |
| X37 | 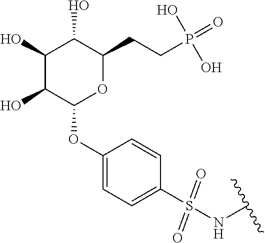 [SMILES: *NS(=O)(=O)c1ccc(O[C@H]2O[C@H](CCP(=O)(O)O)[C@@H](O)[C@H](O)[C@@H]2O)cc1] | 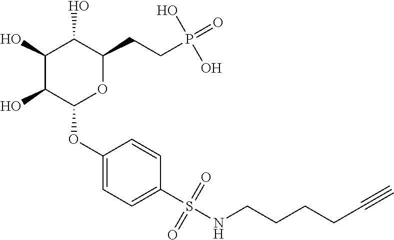 [SMILES: C#CCCCCNS(=O)(=O)c1ccc(O[C@H]2O[C@H](CCP(=O)(O)O)[C@@H](O)[C@H](O)[C@@H]2O)cc1] |
| X38 | 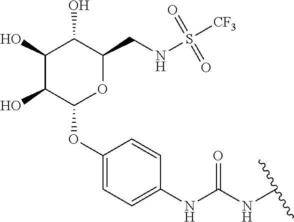 [SMILES: *NC(=O)Nc1ccc(O[C@H]2O[C@H](CNS(=O)(=O)C(F)(F)F)[C@@H](O)[C@H](O)[C@@H]2O)cc1] | 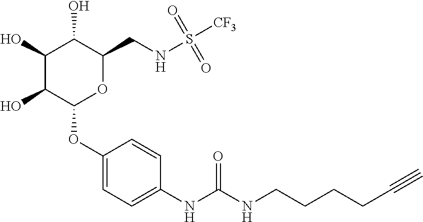 [SMILES: C#CCCCCNC(=O)Nc1ccc(O[C@H]2O[C@H](CNS(=O)(=O)C(F)(F)F)[C@@H](O)[C@H](O)[C@@H]2O)cc1] |
| X27* | 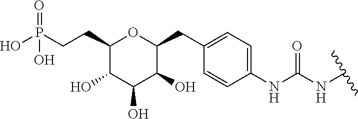 [SMILES: *NC(=O)Nc1ccc(C[C@@H]2O[C@H](CCP(=O)(O)O)[C@@H](O)[C@H](O)[C@@H]2O)cc1] | 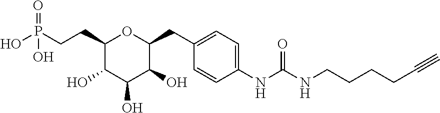 [SMILES: C#CCCCCNC(=O)Nc1ccc(C[C@@H]2O[C@H](CCP(=O)(O)O)[C@@H](O)[C@H](O)[C@@H]2O)cc1] |
| X39* | 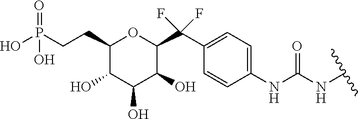 [SMILES: *NC(=O)Nc1ccc(C(F)(F)[C@@H]2O[C@H](CCP(=O)(O)O)[C@@H](O)[C@H](O)[C@@H]2O)cc1] | 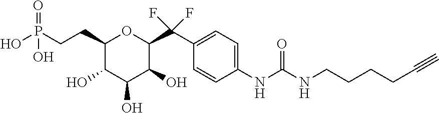 [SMILES: C#CCCCCNC(=O)Nc1ccc(C(F)(F)[C@@H]2O[C@H](CCP(=O)(O)O)[C@@H](O)[C@H](O)[C@@H]2O)cc1] |
| X39 | 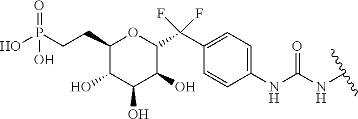 | 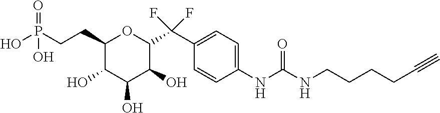 |
| X2* | 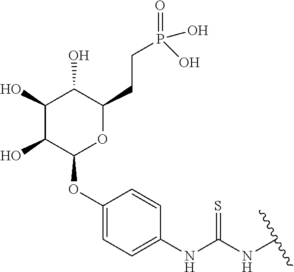 [SMILES: *NC(=S)Nc1ccc(O[C@@H]2O[C@H](CCP(=O)(O)O)[C@@H](O)[C@H](O)[C@@H]2O)cc1] | 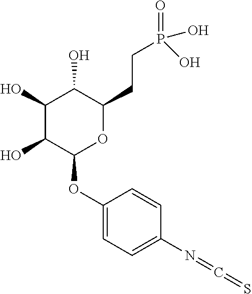 [SMILES: O=P(O)(O)CC[C@H]1O[C@@H](Oc2ccc(N=C=S)cc2)[C@@H](O)[C@@H](O)[C@@H]1O] |
| | | 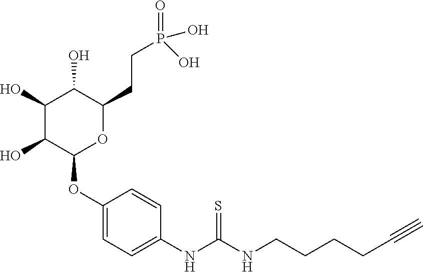 [SMILES: C#CCCCCNC(=S)Nc1ccc(O[C@@H]2O[C@H](CCP(=O)(O)O)[C@@H](O)[C@H](O)[C@@H]2O)cc1] |
| X3* | 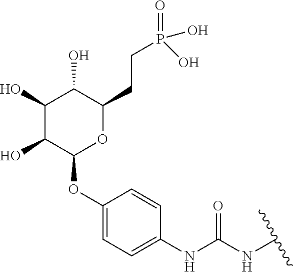 [SMILES: *NC(=O)Nc1ccc(O[C@@H]2O[C@H](CCP(=O)(O)O)[C@@H](O)[C@H](O)[C@@H]2O)cc1] | 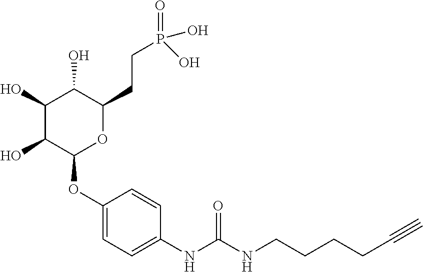 [SMILES: C#CCCCCNC(=O)Nc1ccc(O[C@@H]2O[C@H](CCP(=O)(O)O)[C@@H](O)[C@H](O)[C@@H]2O)cc1] |

Other M6PR binding moieties of interest and synthons or synthetic precursors thereof, are shown in Table 3. X101-X103 show compounds having a phosphate ester or thiophosphate ester head group. X109-X110 show exemplary compounds of formula (V). In some embodiments, such M6PR binding moieties are used in reference compounds for the assessment of compounds of formula (XII).

TABLE 3
Other Exemplary M6PR binding moieties and synthetic precursors

| Exemplary X for M6PR binding compounds | | Exemplary Synthetic precursors |
|---|---|---|
| # | Structure | Structure |
| X101 | 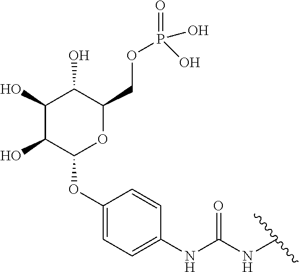 [SMILES: *NC(=O)Nc1ccc(O[C@H]2O[C@H](COP(=O)(O)O)[C@@H](O)[C@H](O)[C@@H]2O)cc1] | 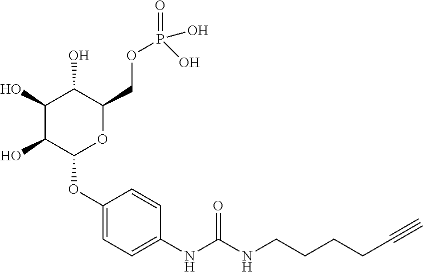 [SMILES: C#CCCCCNC(=O)Nc1ccc(O[C@H]2O[C@H](COP(=O)(O)O)[C@@H](O)[C@H](O)[C@@H]2O)cc1] |
| X102 | 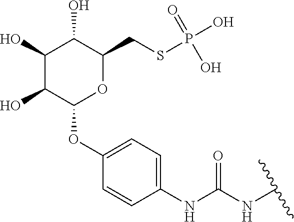 [SMILES: *NC(=O)Nc1ccc(O[C@H]2O[C@H](CSP(=O)(O)O)[C@@H](O)[C@H](O)[C@@H]2O)cc1] | 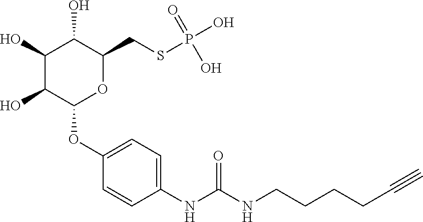 [SMILES: C#CCCCCNC(=O)Nc1ccc(O[C@H]2O[C@H](CSP(=O)(O)O)[C@@H](O)[C@H](O)[C@@H]2O)cc1] |
| X103 | 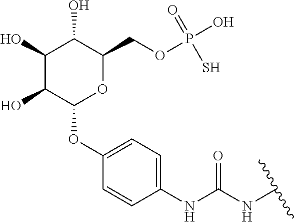 [SMILES: *NC(=O)Nc1ccc(O[C@H]2O[C@H](COP(=O)(O)S)[C@@H](O)[C@H](O)[C@@H]2O)cc1] | 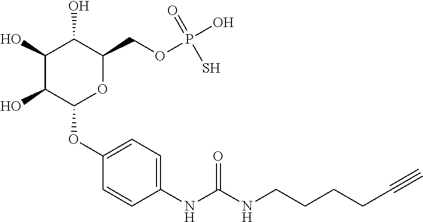 [SMILES: C#CCCCCNC(=O)Nc1ccc(O[C@H]2O[C@H](COP(=O)(O)S)[C@@H](O)[C@H](O)[C@@H]2O)cc1] |
| X104 | 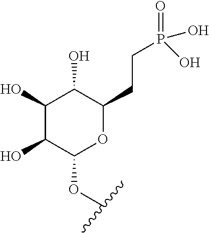 [SMILES: *O[C@H]1O[C@H](CCP(=O)(O)O)[C@@H](O)[C@H](O)[C@@H]1O] | 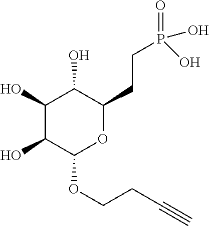 [SMILES: C#CCCO[C@H]1O[C@H](CCP(=O)(O)O)[C@@H](O)[C@H](O)[C@@H]1O] |
| X105 | 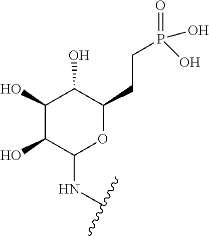 [SMILES: *NC1O[C@H](CCP(=O)(O)O)[C@@H](O)[C@H](O)[C@@H]1O] | 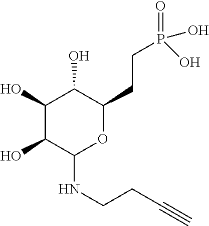 [SMILES: C#CCCNC1O[C@H](CCP(=O)(O)O)[C@@H](O)[C@H](O)[C@@H]1O] |
| X106 | 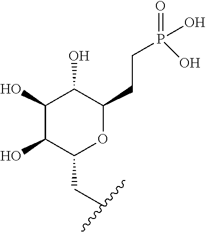 [SMILES: *C[C@H]1O[C@H](CCP(=O)(O)O)[C@@H](O)[C@H](O)[C@@H]1O] | 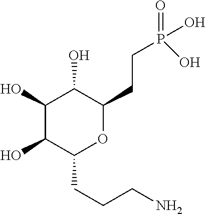 [SMILES: NCCC[C@H]1O[C@H](CCP(=O)(O)O)[C@@H](O)[C@H](O)[C@@H]1O] |
| X107 | 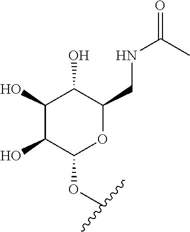 [SMILES: *O[C@H]1O[C@H](CNC(C)=O)[C@@H](O)[C@H](O)[C@@H]1O] | 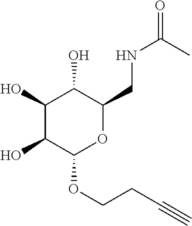 [SMILES: C#CCCO[C@H]1O[C@H](CNC(C)=O)[C@@H](O)[C@H](O)[C@@H]1O] |
| X108 | 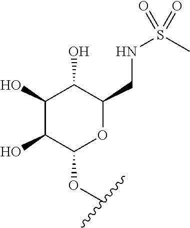 [SMILES: *O[C@H]1O[C@H](CNS(C)(=O)=O)[C@@H](O)[C@H](O)[C@@H]1O] | 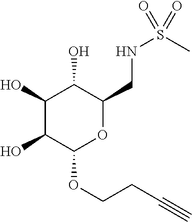 [SMILES: C#CCCO[C@H]1O[C@H](CNS(C)(=O)=O)[C@@H](O)[C@H](O)[C@@H]1O] |
| X109 | 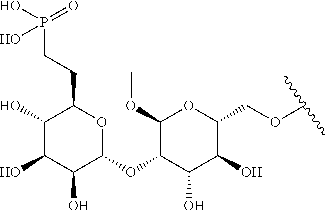 [SMILES: *OC[C@H]1O[C@H](OC)[C@@H](O[C@H]2O[C@H](CCP(=O)(O)O)[C@@H](O)[C@H](O)[C@@H]2O)[C@@H](O)[C@@H]1O] | 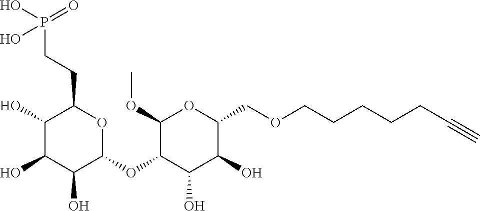 [SMILES: C#CCCCCCOC[C@H]1O[C@H](OC)[C@@H](O[C@H]2O[C@H](CCP(=O)(O)O)[C@@H](O)[C@H](O)[C@@H]2O)[C@@H](O)[C@@H]1O] |
| X110 | 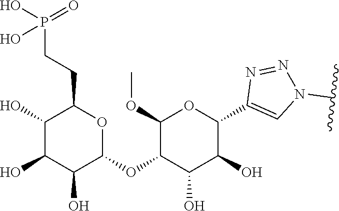 [SMILES: *n1cc([C@H]2O[C@H](OC)[C@@H](O[C@H]3O[C@H](CCP(=O)(O)O)[C@@H](O)[C@H](O)[C@@H]3O)[C@@H](O)[C@@H]2O)nn1] | 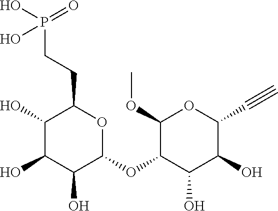 [SMILES: C#C[C@H]1O[C@H](OC)[C@@H](O[C@H]2O[C@H](CCP(=O)(O)O)[C@@H](O)[C@H](O)[C@@H]2O)[C@@H](O)[C@@H]1O] |

### 4.2.5. Disaccharide Containing M6PR Binding Moieties

Aspects of this disclosure include compounds and conjugates of formula (I) having a M6PR binding moiety including a particular di-mannose structure having a first pyranose ring (e.g., of formula (II)) connected to a second 2,5-linked pyranose ring that is further connected to the linker.

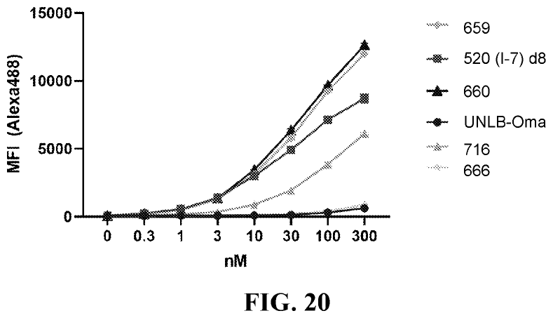

FIG. 20 shows select cellular uptake activity illustrating a comparison between a compound of formula (III) conjugate and a compound having a particular di-mannose M6PR binding moiety. Conjugates of M6PR binding compounds 660 or 659, each having a di-mannose structure with a 2,5-linked pyranose ring connected to the linker, showed potent and comparable activity to conjugate of a conjugate of compound 520 (1-7).

Accordingly, aspects of this disclosure include cell surface M6PR binding compounds of formula (XV):

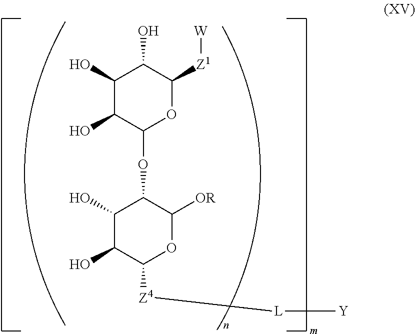

or a prodrug thereof, or a salt thereof, wherein:
W is a non-hydrolyzable hydrophilic head group;
Z¹ is selected from optionally substituted (C₁-C₃)alkylene and optionally substituted ethenylene;
Z⁴ is selected from —Z¹⁴—, —Z¹⁴-A-, -A-, and —CH₂—Z¹⁴—,
Z¹⁴ is selected from O, S, NR²¹, and C(R²²)₂, wherein R²¹ is independently selected from H, and optionally substituted (C₁-C₆)alkyl, and each R²² is independently selected from H, halogen (e.g., F) and optionally substituted (C₁-C₆)alkyl;
A is an optionally substituted cyclic group (e.g., optionally substituted aryl, optionally substituted heteroaryl, optionally substituted heterocycle, optionally substituted cycloalkyl);
n is 1 to 500;
m is 1 to 500;
L is a linker; and
Y is a moiety of interest.

In some embodiments of formula (XV), Z⁴ is —CH₂—Z¹⁴—, wherein Z¹⁴ is selected from O, S, NR²¹, and C(R²²)₂.

In some embodiments of formula (XV), Z⁴ is —CH₂-A-.

In some embodiments of formula (XV), Z⁴ is -A-.

In some embodiments of formula (XV), A is cyclic group (e.g., an optionally substituted aryl, or optionally substituted heteroaryl, e.g., as described above for formula (III)). In some embodiments of formula (XV), A is a cyclic group as defined above in Formula (III).

In some embodiments of formula (XV), A is triazole.

In some embodiments of formula (XV), Z⁴ is

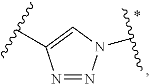 [SMILES: *c1cn(*)nn1]

wherein “*” denotes a connection to the linker L.

The M6PR binding moieties of formula (XV) can be adapted for use in a variety of compounds and conjugates as described herein.

In some embodiments of formula (XV), m is 1 to 100, such as 1-5, 5-10, 10-20, 10-100, 20-80, or 20-50. In some embodiments of formula (XV), m is 1, 2, 3, 4 or 5.

### 4.2.6. Prodrugs

Aspects of this disclosure include prodrugs of any of the M6PR binding moieties described herein that are incorporated into the compounds and conjugates of this disclosure.

The term “prodrug” refers to an agent which is converted into the drug in vivo by some physiological or chemical process (e.g., a prodrug on being brought to the physiological pH is converted to the desired drug form).

Prodrugs forms of any of the M6PR binding moieties described herein can be useful because, for example, can lead to particular therapeutic benefits as a consequence of an extension of the half-life of the resulting compound or conjugate in the body or a reduction in the active dose required.

Pro-drugs can also be useful in some situations, as they may be easier to administer than the parent drug. They may, for instance, be bioavailable by oral administration whereas the parent drug is not. The pro-drug may also have improved solubility in pharmacological compositions over the parent drug.

A prodrug derivative of a M6PR binding moiety generally includes a promoiety substituent at a suitable labile site of the compound, e.g., a hydroxy group of the pyranose ring of formula (II). The promoiety refers to the group that is removed by enzymatic or chemical reactions, when a prodrug is converted to the drug in vivo. For example, a promoiety can be an optionally substituted alkyl acyl group attached to a hydroxy group of the compound via an ester linkage. Exemplary alkyl acyl promoiety groups include acetyl. In some embodiments, a prodrug derivative of one or more of the hydroxyl groups of the pyranose sugar ring may be incorporated into the compounds. For example, an ester promoiety can be incorporated at one or more hydroxyl groups at the 2, 3 and/or 4 positions of the sugar ring.

In some embodiments, a prodrug derivative of the hydrophilic head group (W) may be incorporated into the M6PR binding moieties and compounds of this disclosure. For example, an ester promoiety can be incorporated onto a phosphonate, or thiophosphonate head group, or an ester promoiety can be incorporated onto a carboxylic acid or malonic acid head group.

### 4.3. Linkers

The terms “linker”, “linking moiety” and “linking group” are used interchangeably and refer to a linking moiety that covalently connects two or more moieties or compounds, such as M6PR binding moieties and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a cell surface M6PR. When Y is a polypeptide, the polypeptide conjugation reactive terminus of the linker is in some cases a site that is capable of conjugation to the polypeptide through a cysteine thiol or lysine amine group on the polypeptide, and so can be a thiol-reactive group such as a maleimide or a dibromomaleimide, or as defined herein, or an amine-reactive group such as an active ester (e.g., pentafluorophenyl ester or tetrafluorophenyl ester or NHS ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to a cell surface M6PR binding moiety (X), optionally substituted heteroarylene linked to X, optionally substituted heterocyclene linked to X, or optionally substituted cycloalkylene linked to X. In certain embodiments, L is a linker between about 10 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 400 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 200 Å, which comprises an optionally substituted arylene linked to X, optionally substituted heteroarylene linked to X, optionally substituted heterocyclene linked to X, or optionally substituted cycloalkylene linked to X.

In certain embodiments, L separates cell surface M6PR binding moiety (Y) and Y (or Z) by a backbone comprising at least 10 consecutive atoms. In certain cases, the backbone is at least 12 consecutive atoms. In certain cases, the backbone is at least 14 consecutive atoms. In certain cases, the backbone is at least 16 consecutive atoms. In certain cases, the backbone is at least 18 consecutive atoms. In certain cases, the backbone is at least 20 consecutive atoms. In certain cases, the backbone is at least 22 consecutive atoms. In certain cases, the backbone is at least 24 consecutive atoms. In certain cases, the backbone is at least 26 consecutive atoms. In certain cases, the backbone is at least 28 consecutive atoms. In certain cases, the backbone is at least 30 consecutive atoms. In certain cases, the backbone is at least 32 consecutive atoms. In certain cases, the backbone is at least 34 consecutive atoms. In certain cases, the backbone is at least 36 consecutive atoms. In certain cases, the backbone is at least 38 consecutive atoms. In certain cases, the backbone is at least 40 consecutive atoms. In certain cases, the backbone is up to 50 consecutive atoms. In certain cases, the backbone is up to 60 consecutive atoms. In certain cases, the backbone is up to 70 consecutive atoms. In certain cases, the backbone is up to 80 consecutive atoms. In certain cases, the backbone is up to 90 consecutive atoms. In certain cases, the backbone is up to 100 consecutive atoms.

In certain embodiments, linker L separates cell surface M6PR binding moiety (X) and Y (or Z) by a chain of 4 to 500 consecutive atoms. In certain embodiments, linker L separates X and Y (or Z) by a chain of 4 to 50 consecutive atoms. In certain embodiments, linker L separates X and Y (or Z) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z) by a chain of 6 to 50 consecutive atoms. In certain embodiments, linker L separates X and Y (or Z) by a chain of 11 to 50 consecutive atoms. In certain embodiments, linker L separates X and Y (or Z) by a chain of 16 to 50 consecutive atoms. In certain embodiments, linker L separates X and Y (or Z) by a chain of 21 to 50 consecutive atoms. In certain embodiments, linker L separates X and Y (or Z) by a chain of 26 to 50 consecutive atoms. In certain embodiments, linker L separates X and Y (or Z) by a chain of 31 to 50 consecutive atoms. In certain embodiments, linker L separates X and Y (or Z) by a chain of 36 to 50 consecutive atoms. In certain embodiments, linker L separates X and Y (or Z) by a chain of 41 to 50 consecutive atoms. In certain embodiments, linker L separates X and Y (or Z) by a chain of 46 to 50 consecutive atoms.

In certain embodiments, linker L separates X and Y (or Z) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L separates X and Y (or Z) by a chain of 50 or 55 consecutive atoms, by a chain of 56 to 60 consecutive atoms, by a chain of 61 to 65 consecutive atoms, by a chain of 66 to 70 consecutive atoms, by a chain of 71 to 75 consecutive atoms, by a chain of 76 to 80 consecutive atoms, by a chain of 81 to 85 consecutive atoms, by a chain of 86 to 90 consecutive atoms, by a chain of 91 to 95 consecutive atoms, or by a chain of 96 to 100 consecutive atoms.

In certain embodiments, linker L is a chain of 5 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 7 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 10 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 15 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

In certain embodiments, linker L is a chain of 5 to 500 consecutive atoms separating X and Y (or Z) and which comprises an optionally substituted arylene linked to X or optionally substituted heteroarylene linked to X. In certain embodiments, linker L is a chain of 7 to 500 consecutive atoms separating X and Y (or Z) and which comprises an optionally substituted arylene linked to X or optionally substituted heteroarylene linked to X. In certain embodiments, linker L is a chain of 10 to 500 consecutive atoms separating X and Y (or Z) and which comprises an optionally substituted arylene linked to X or optionally substituted heteroarylene linked to X. In certain embodiments, linker L is a chain of 15 to 400 consecutive atoms separating X and Y (or Z) and which comprises an optionally substituted arylene linked to X or optionally substituted heteroarylene linked to X.

In certain embodiments, linker L is a chain of 5 to 500 consecutive atoms separating X and Y (or Z) and which comprises an optionally substituted phenylene linked to X. In certain embodiments, linker L is a chain of 7 to 500 consecutive atoms separating X and Y (or Z) and which comprises an optionally substituted phenylene linked to X. In certain embodiments, linker L is a chain of 10 to 500 consecutive atoms separating X and Y (or Z) and which comprises an optionally substituted phenylene linked to X. In certain embodiments, linker L is a chain of 15 to 400 consecutive atoms separating X and Y (or Z) and which comprises an optionally phenylene linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

It is understood that the linker may be considered as connecting directly to a Z³ or Z⁴ group of a M6PR binding moiety (X) (e.g., as described herein). In some embodiments of formula (III) or (V), the linker may be considered as connecting directly to the Z³ or Z⁴ group. Alternatively, a —Z³-L-group or —Z⁴-L¹- of the linker formula (e.g., as described herein) can be considered part of a linking moiety that connects Z³ or Z⁴ to Y. The disclosure is meant to include all such configurations of M6PR binding moiety (X) and linker (L).

In some embodiments of formula (XI)-(XIII), L is a linker of formula (VII):

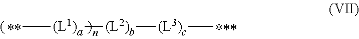

wherein
L¹ and L³ are independently a linker, and L² is a branched linking moiety, wherein L¹ to L³ together provide a linear or branched linker between X and Y;
a, b and c are independently 0 or 1;
** represents the point of attachment to L¹ of X via Z¹; and
*** represents the point of attachment to Y;
wherein:
when n is 1, a is 1, and b is 0;
when n is >1, a is 1, and b is 1.

In certain embodiments of the linker of formula (VII), L¹ to L³ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non-naturally occurring amino acid residue), —NH—, and —NMe-, wherein each p is independently 1 to 50.

In certain embodiments of the linker of formula (VII), any of L¹-L³ comprises repeating ethylene glycol moieties (e.g., —CH₂CH₂O- or —OCH₂CH₂—). In certain cases, the linker of formula (VII) comprises 1 to 25 ethylene glycol moieties, such as 3 to 25, 5 to 25, 7 to 25, 10 to 25, 15 to 25, 17 to 25, 20 to 25 or 22 to 25 ethylene glycol moieties. In some instances, the linker of formulae (VII) comprises 3 or more ethylene glycol moieties, such as 5 or more, 7 or more, 10 or more, 15 or more, 20 or more, or even more ethylene glycol moieties.

In certain embodiments of the linker of formula (VII), any of L¹-L³ comprises one or more triazole linking moieties. In some instances, the linker comprises one or more 1,2,3-triazole linking moieties. In certain cases, the one or more 1,2,3-triazole moieties is selected from one of the following structures:

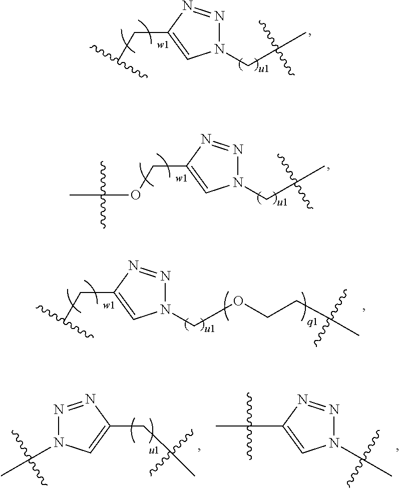

wherein w1, u1 and q1 are independently 1 to 25 (e.g., 1 to 12, such as 1 to 6).

In certain embodiments of the linker of formula (VII), n is 1, such that b is 0, and the linker is of the formula (VIIa):

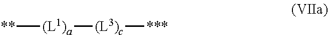

wherein
   L¹ and L³ are independently a linker (e.g., as described herein), wherein L¹ to L³ together provide a linear linker between X and Y;
   a is 1;
   c is 0 or 1;
   ** represents the point of attachment to L¹ of X via Z¹; and
   *** represents the point of attachment to Y.

In certain embodiments of the linker of formula (VIIa), the linear linker has a backbone of 20 or more consecutive atoms covalently linking X to Y via Z¹, such as a backbone of 25 or more consecutive atoms, or 30 or more consecutive atoms, and in some cases, up to 100 consecutive atoms. In certain embodiments of formula (VIIa), the linear linker separates X and Y (or Z¹) by a chain of 20 to 50 consecutive atoms. In certain embodiments, the linear linker separates X and Y (or Z¹) by a chain of 21 to 50 consecutive atoms, by a chain of 22 to 50 consecutive atoms, by a chain of 23 to 50 consecutive atoms, by a chain of 24 to 50 consecutive atoms, by a chain of 25 to 50 consecutive atoms, by a chain of 26 to 50 consecutive atoms, by a chain of 27 to 50 consecutive atoms, by a chain of 28 to 50 consecutive atoms, or by a chain of 29 to 50 consecutive atoms. In certain embodiments of formula (VIIa), the linear linker separates X and Y (or Z¹) by a chain of 30 to 60 consecutive atoms. In certain embodiments, the linear linker separates X and Y (or Z¹) by a chain of 31 to 60 consecutive atoms. In certain embodiments, the linear linker separates X and Y (or Z¹) by a chain of 32 to 60 consecutive atoms. In certain embodiments, the linear linker separates X and Y (or Z¹) by a chain of 33 to 60 consecutive atoms. In certain embodiments, the linear linker separates X and Y (or Z¹) by a chain of 34 to 60 consecutive atoms. In certain embodiments, the linear linker L separates X and Y (or Z¹) by a chain of 35 to 50 consecutive atoms. In certain embodiments, the linear linker L separates X and Y (or Z¹) by a chain of 36 to 50 consecutive atoms. In certain embodiments, the linear linker L separates X and Y (or Z¹) by a chain of 41 to 50 consecutive atoms. In certain embodiments, the linear linker L separates X and Y (or Z¹) by a chain of 46 to 50 consecutive atoms.

In certain other embodiments of formula (VII), n is 2 or more, such that L¹ to L³ together provide a branched linker between X and Y.

In certain embodiments of formula (VII), n is 2 or more, and L² is selected from:

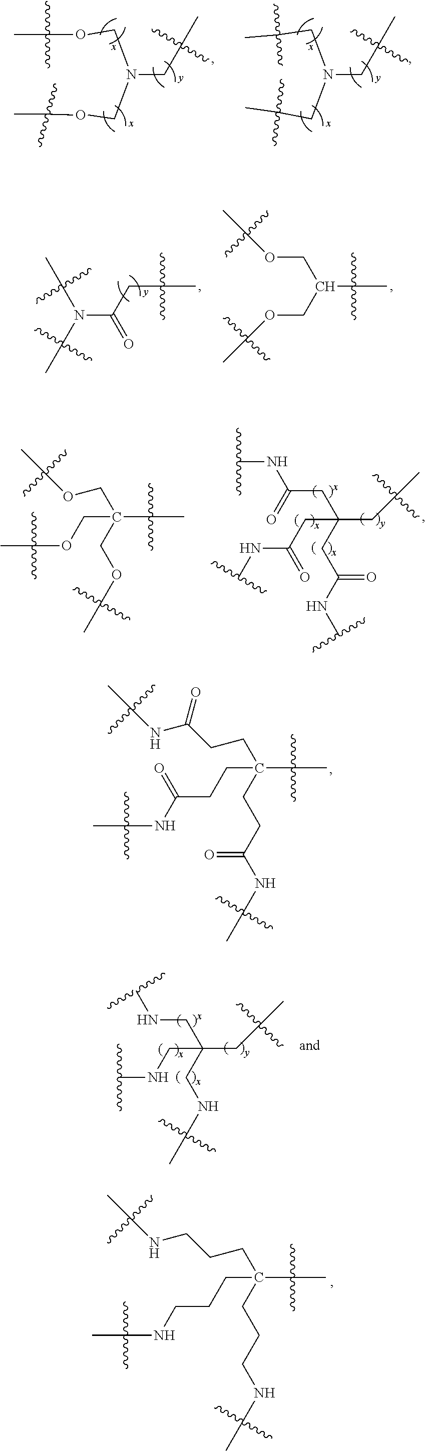

wherein each x and y are independently 1 to 10.

In certain embodiments of formula (VII), L¹-L² comprises a backbone of 14 or more consecutive atoms between X and the branching atom, such as 14 to 50, 14 to 40, 14 to 35 or 14 to 30 consecutive atoms between X and the branching atom.

In certain embodiments of formula (VII) or (VIIa), L³ comprises a backbone of 10 to 80 consecutive atoms, such as 12 to 70, 12 to 60, or 12 to 50 consecutive atoms.

In certain embodiments of formula (VII) or (VIIa), wherein L³ comprises a linking moiety selected from (C₁₀-C₂₀-alkylene (e.g., C₁₂-alkylene), or —(OCH₂CH₂)ₚ—, where p is 1 to 25, such as 3 to 25, 5 to 24, 7 to 25, 10 to 25, 15 to 25 or 20 to 24.

In certain embodiments, L is of formula (VIIb):

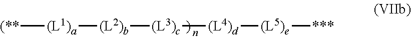

wherein each L¹ to L⁵ is independently a linking moiety which together provide a linear or branched linker between Z¹ and Y;
a, b, c, d, and e are each independently 0, 1, or 2;
   ** represents the point of attachment to L¹ of X via Z¹; and
   *** represents the point of attachment to Y;
wherein:
   when n is 1, a is 1, and c is 0; and
   when n is >1, a is 1, and c is 1.

In certain embodiments of the linker of formula (VIIb), L¹ to L⁵ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non-naturally occurring amino acid residue), —NH—, and —NMe-, wherein each p is independently 1 to 50.

In certain embodiments of formula (VIIb), -(L¹)ₐ- comprises an optionally substituted alkyl or ethylene glycol linking moiety. In certain cases, L¹ comprises an optionally substituted —C₁₋₆-alkylene-. In certain cases, L¹ comprises an ethylene glycol linking moiety.

In certain embodiments of formula (VIIb), L¹ is independently selected from: —C₁₋₆-alkylene-, —(CH₂CH₂O)₁—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl. In certain cases, L¹ is —C₁₋₆-alkylene-, such as —C₁₋₃-alkylene-. In certain cases, L¹ is —(CH₂CH₂O)ₜ—, where t is 1 to 20, such as 1 to 15, 1 to 10, 1 to 8, 1 to 6, or 1 to 4. In certain cases, L¹ is —C₁₋₆-alkylene-NR⁴CO—. In certain cases, L¹ is —C₁₋₆-alkyleneCONH—. In certain cases, L¹ is or OCH₂.

In some embodiments of formula (VIIb), one or more L¹ is independently —CH₂O—; —(CH₂CH₂O)ₜ—, —NR⁴CO—, —C₁₋₆-alkylene-,

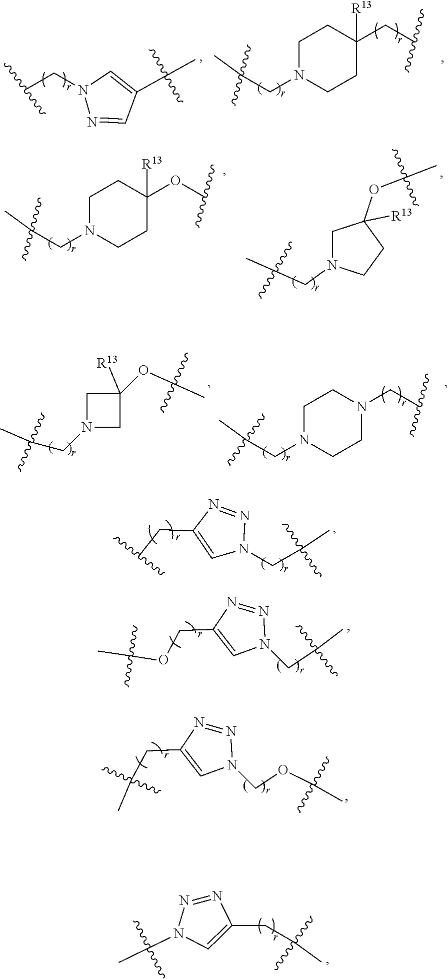

wherein: R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
each r independently 0 to 20, and any of the L¹ moieties are optionally further substituted.

In certain embodiments of formula (VIIb), L² is independently selected from: —NR⁴CO—C₁₋₆-alkylene-, —CONR⁴—C₁₋₆-alkylene,

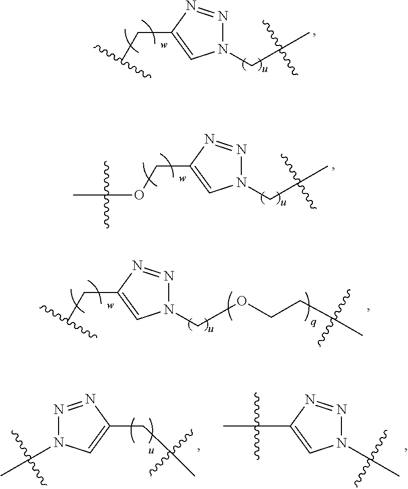

—OCH₂—, and (OCH₂CH₂)_{q}—, wherein q is 1 to 10, u is 0 to 10, w is 1 to 10, and R⁴ is independently selected from H, and optionally substituted (C₁-C₆)alkyl. In certain cases, L² is —NR⁴CO—C₁₋₆-alkylene-. In certain cases, L² is —CONR⁴—C₁₋₆-alkylene.

In certain cases, L² is

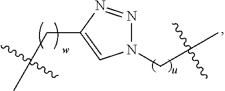

where w is 1 and u is 0 or 1.

In certain cases, L² is

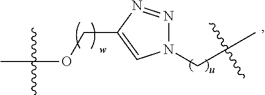

where w is 1 and u is 0 or 1.

In certain cases, L² is

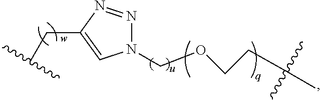

where w is 1, u is 0 or 1, and q is 1.

In certain cases, L² is

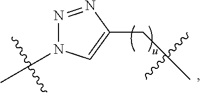

where u is 0 or 1.

In certain cases, L² is

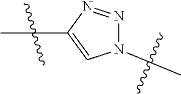 [SMILES: *c1cn(*)nn1]

In certain embodiments, L² is —OCH₂—. In certain other embodiments, L² is (OCH₂CH₂)_{q}, and q is 1 to 10, 1 to 9, 1 to 8, 1 to 7, 1 to 6, 1 to 5, 1 to 4, 1 to 3 or 1 to 2. In certain cases, q is 2 to 8, such as 2 to 6, 4 to 6, or 2 to 4.

In certain embodiments of formula (VIIb), L⁴ is absent or independently selected from —C₁₋₆-alkylene-, —(CH₂CH₂O)—, —C₁₋₆-alkylene-NHCO—, —C₁₋₆-alkyleneCONH—, or OCH₂, wherein t is 1 to 20. In certain cases, L⁴ is absent. In certain cases, L⁴ is —C₁₋₆-alkylene-. In certain cases, L⁴ is —(CH₂CH₂O)ₜ—, where t is 1 to 20, such as 1 to 15, 1 to 12, 1 to 10, 1 to 8, 1 to 6, 1 to 4 or 1 to 3. In certain cases, L⁴ is —C₁₋₆-alkylene-NHCO—. In certain cases, L⁴ is —C₁₋₆-alkyleneCONH—. In certain cases, L⁴ is OCH₂.

In some embodiments of the subject compounds, n is 1 and L³ in formula (VIIb) is absent.

In certain embodiments of the subject compounds, n is 2 or more, and L³ of formula (VIIb) is a branched linking moiety.

Accordingly, in some embodiments of formula (VIIb), L³ is a branched linking moiety, e.g., a trivalent linking moiety. For example, an L³ linking moiety can be of the one of the following general formula:

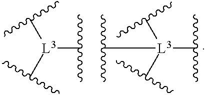

In some embodiments of formula (VIIb), the branched linking moiety can be of higher valency and be described by one of the one of the following general formula:

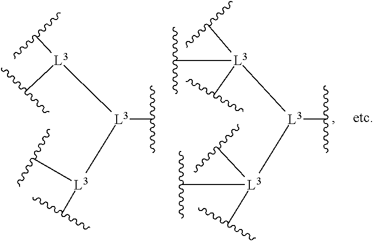

where any two L³ groups can be directed linked or connected via optional linear linking moieties (e.g., as described herein).

In some embodiments of formula (VIIb), the branched linking moiety can include one, two or more L³ linking moieties, each being trivalent moieties, which when linked together can provide for multiple branching points for covalent attachment of the ligands and be described by one of the one of the following general formula:

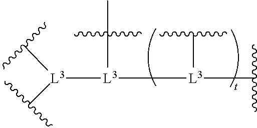

where t is 0 to 500, such as 0 to 100, 0 to 20, or 0 to 10.

In some embodiments, the branched linking moiety (e.g., L³) comprises one or more of: an amino acid residue (e.g., Asp, Lys, Orn, Glu, Ser), N-substituted amido (—N(−)C(═O)—), tertiary amino, polyol (e.g., O-substituted glycerol), and the like.

In some embodiments of formula (VIIb), one or more L³ is a branching moiety selected from

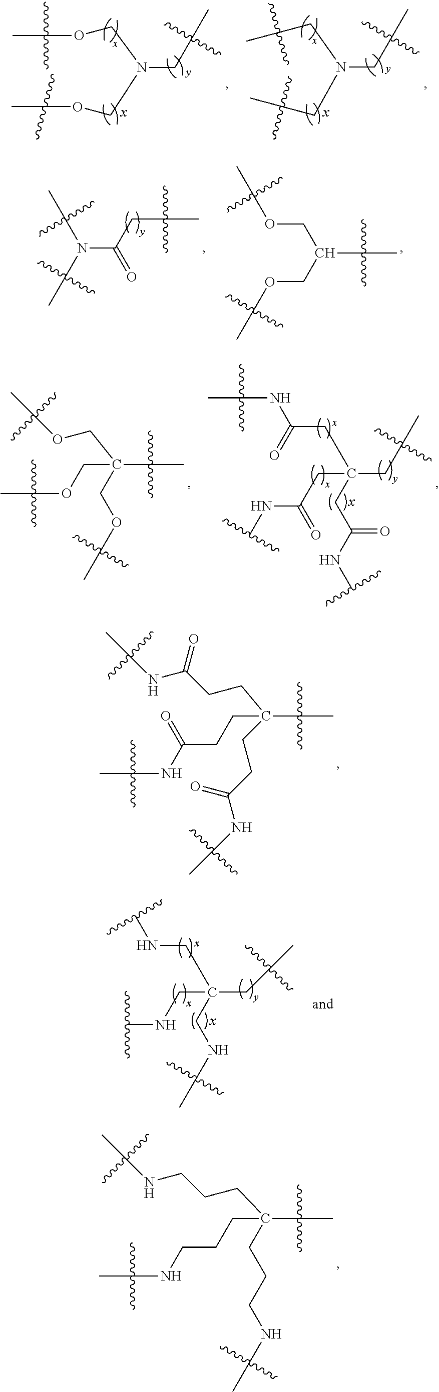

wherein each x and y are each independently 1 to 10, such as 1-6, 1-3, e.g., 1 or 2. In some cases, each x is 1, 2 or 3, e.g., 2.

In some embodiments of formula (VIIb), one or more L⁵ is independently —CH₂O—; —(CH₂CH₂O)ₜ—, —NR⁴CO—, —C₁₋₆-alkylene-,

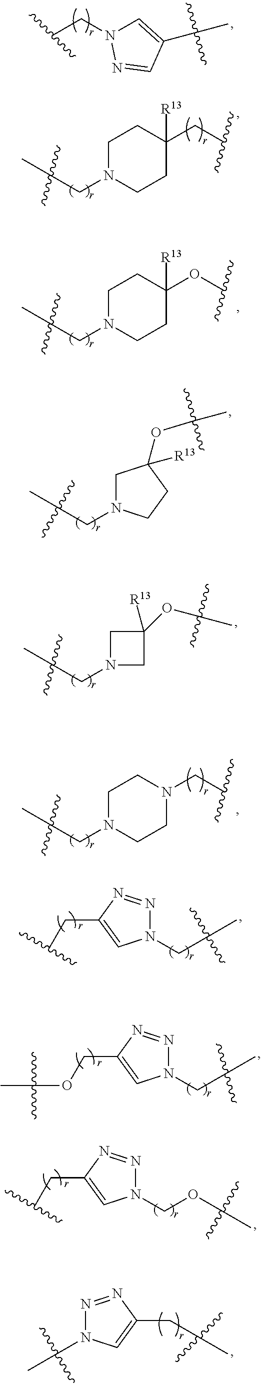

wherein: R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
each r independently 0 to 20, and any of the L⁵ moieties are optionally further substituted.

In certain cases, L⁵ is —CH₂O—. In certain cases, L⁵ is —(CH₂CH₂O)ₜ—, where t is 1 to 20, such as 1-15, 1-12, 1-10, 1-8, 1-6, or 1 to 4. In certain cases, L⁵ is —NR⁴CO—, where R⁴ is H, or optionally substituted (C₁-C₆)alkyl. In certain cases, L⁵ is —C₁₋₆-alkylene-.

In certain cases, L⁵ is

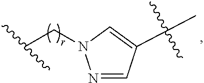

where r is 0 to 20, such as 0 to 15, 0 to 10, 0 to 8, or 0 to 5.

In certain cases, L⁵ is

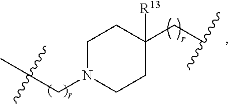

where each r is independently 0 to 20, such as 0 to 15, 0 to 10, 0 to 8, or 0 to 5 and R¹³ is H, or optionally substituted (C₁-C₆)alkyl.

In certain cases, L⁵ is

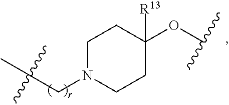

where r is 0 to 20, such as 0 to 15, 0 to 10, 0 to 8, or 0 to 5 and R¹³ is H, or optionally substituted (C₁-C₆)alkyl.

In certain cases, L⁵ is

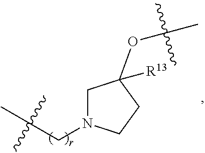

where r is 0 to 20, such as 0 to 15, 0 to 10, 0 to 8, or 0 to 5, and R¹³ is H, or optionally substituted (C₁-C₆)alkyl.

In certain cases, L⁵ is

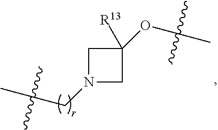

where r is 0 to 20, such as 0 to 15, 0 to 10, 0 to 8, or 0 to 5, and R¹³ is H, or optionally substituted (C₁-C₆)alkyl.

In certain cases, L⁵ is

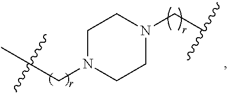

where each r is independently 0 to 20, such as 0 to 15, 0 to 10, 0 to 8, or 0 to 5.

In certain cases, L⁵ is

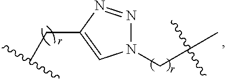

where each r is independently 0 to 20, such as 0 to 15, 0 to 10, 0 to 8, or 0 to 5.

In certain cases, L⁵ is

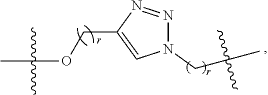

where each r is independently 0 to 20, such as 0 to 15, 0 to 10, 0 to 8, or 0 to 5.

In certain cases, L⁵ is

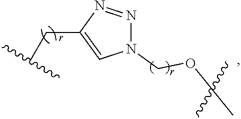

where each r is independently 0 to 20, such as 0 to 15, 0 to 10, 0 to 8, or 0 to 5.

In certain cases, L⁵ is

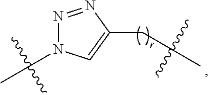

where r is 0 to 20, such as 0 to 15, 0 to 10, 0 to 8, or 0 to 5.

In certain embodiments of formula (VIIb), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In some embodiments of formula (VIIb), L⁵ comprises one or more of: an amino acid residue (e.g., Asp, Lys, Orn, Glu, Ser), an amino acid analogue, N-substituted amido (—N(−)C(═O)—), tertiary amino, polyol (e.g., O-substituted glycerol), and the like. Analogs of an amino acid, include but not limited to, unnatural amino acids, as well as other modifications known in the art. The amino acid includes L-amino acids, D-amino acids, or both, and may contain any of a variety of amino acid modifications or analogs known in the art.

In some embodiments of formula (VIIb), L¹-L⁵ comprises one or more of the following units:

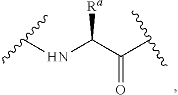

where R^{a} is (C₁-C₆)alkyl or substituted (C₁-C₆)alkyl, e.g., a (C₁-C₆)alkyl optionally substituted with amine, a tertiary amine, optionally substituted alkoxy, optionally substituted carboxyl, optionally substituted aryl, or optionally substituted heteroaryl. It is understood that R^{a} can be linked to a M6PR binding moiety.

In certain embodiments of formula (VIIb), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In certain embodiments of formula (VII), (VIIa) or (VIIb), the linker comprises 20 to 100 consecutive atoms, such as 20 to 90, 20 to 80, 20 to 70, 20 to 60, 20 to 50, 20 to 40 or 20 to 30 consecutive atoms. In certain cases, the linker comprises 25 to 100 consecutive atoms, such as 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive atoms.

In certain embodiments of formula (VII), (VIIa) or (VIIb), the linker comprises 25 or more consecutive atoms, such as 26 or more, 27 or more, 28 or more, 29 or more or 30 or more consecutive atoms. In certain embodiments of formula (VII), (VIIa) or (VIIb), the linker comprises 30 or more consecutive atoms, such as 31 or more, 32 or more, 33 or more, 34 or more, 35 or more, 36 or more, 37, or more, 38 or more, 39 or more, 40 or even more consecutive atoms.

In certain embodiments where the linker of formula (VII) or (VIIb) is a branched linker, each branch of the linker comprises a linear linker of 14 or more consecutive atoms to covalently link via Z¹ each X moiety to a branching point of the linker. In certain cases, each branch of the linker comprises a linear linker of 15 or more consecutive atoms to the branching point. In certain cases, each branch of the linker comprises a linear linker of 16 or more consecutive atoms to the branching point. In certain cases, each branch of the linker comprises a linear linker of 18 or more consecutive atoms to the branching point. In certain cases, each branch of the linker comprises a linear linker of 20 or more consecutive atoms to the branching point. In certain cases, each branch of the linker comprises a linear linker of 22 or more consecutive atoms to the branching point.

In certain embodiments of formula (VII) or (VIIb), the linker is a branched linker comprising branches covalently linking via Z¹ each X moiety to a branching point of the linker, and a linear linker covalently linking the branching point to Y. In certain cases, the linear linker covalently linking the branching point to Y is 12 or more consecutive atoms. In certain cases, the linear linker covalently linking the branching point to Y is 15 or more consecutive atoms. In certain cases, the linear linker covalently linking the branching point to Y is 20 or more consecutive atoms. In certain cases, the linear linker covalently linking the branching point to Y is 25 or more consecutive atoms. In certain cases, the linear linker covalently linking the branching point to Y is 30 or more consecutive atoms. In certain cases, the linear linker covalently linking the branching point to Y is 40 or more consecutive atoms. In certain cases, the linear linker covalently linking the branching point to Y is 50 or more consecutive atoms. In certain cases, the linear linker covalently linking the branching point to Y is 60 or more consecutive atoms. In certain cases, the linear linker covalently linking the branching point to Y is 70 or more consecutive atoms. In certain cases, the linear linker covalently linking the branching point to Y is 80 or more consecutive atoms.

In some embodiments, the linker includes a polypeptide scaffold where some or all of the sidechain groups of the amino acid residues have been modified to attach a M6PR binding moiety (e.g., as described herein). It is understood that M6PR binding moieties (e.g., as described herein) can be conjugated to amino acid residues, such as Asp, Lys, Orn, Glu, and Ser, of a polypeptide containing linker via a convenient conjugation chemistry. In some embodiments, the linker contains a polylysine polypeptide. In some embodiments, the linker contains a polyornithine polypeptide. In some embodiments, the linker contains a polyserine polypeptide. In some embodiments, the linker contains a polyaspartate polypeptide. The polypeptide can be a randomly polymerized polymer having an average length, or a polymer of defined length prepared e.g., in a controlled stepwise fashion. In some cases, the polypeptide linker segment has a length of 10-100 amino acid residues, such as 20-90, or 20-50 amino acid residues. In some embodiments, the N-terminal or C-terminal of the polypeptide linker segment is modified to include a linking unit to an additional M6PR binding moiety (e.g., as described herein). In some embodiments, the N-terminal or C-terminal of the polypeptide linker segment is modified with one or more linking units (e.g., as described herein) suitable for attachment to a Y moiety of interest.

In some embodiments, the linker includes a scaffold of formula (VIIIa) or (VIIIb):

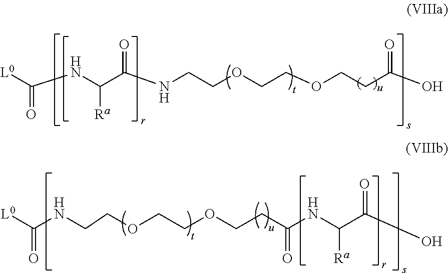

wherein:
L⁰ is a linking moiety (e.g., one or more amino acid residues), a linked M6PR binding moiety, optionally substituted alkyl, or optionally substituted aryl or heteroaryl;
R^{a} is (C₁-C₆)alkyl or substituted (C₁-C₆)alkyl (e.g., a (C₁-C₆)alkyl optionally substituted with amine, a tertiary amine, optionally substituted alkoxy, optionally substituted carboxyl, optionally substituted aryl, or optionally substituted heteroaryl), a derivative of an amino acid sidechain group (e.g., a lysine, serine, aspartate, glutamate, ornithine, etc), or a linked M6PR binding moiety;
r is 1-10 (e.g., r is 1-5);
t is 1-11 (e.g., t is 1-5);
u is 0-5 (e.g., u is 0, 1 or 2); and
s is 1-50 (e.g., s is 1-20, 1-10, or 1-5).

It is understood that the C-terminal carboxylic acid group of formula (VIIIa)-(VIIIb) can provide for coupling (e.g., via a chemoselective ligation group) to a further linking moiety (e.g., one or more amino acid residues), and/or a moiety of interest (Y) (e.g., as described herein).

In some embodiments of (VIIIa) or (VIIIb), r is 1-3. In some embodiments of (VIIIa) or (VIIIb), t is 3-11, such as 3-5. In some embodiments of (VIIIa) or (VIIIb), u is 1. In some embodiments of (VIIIa) or (VIIIb), s is at least 2. In some embodiments of (VIIIa) or (VIIIb), s is 2-10, such as 2-5, e.g., 2, or 3.

In some embodiments of (VIIIa) or (VIIIb), r is 1-3, t is 3-5, u is 0 or 1, and s is 2-5 (e.g., 2, or 3).

In some embodiments of (VIIIa) or (VIIIb), t is 3, s is 1, r is 1, and u is 1. In some embodiments of (VIIIa) or (VIIIb), t is 3, s is 1, r is 1, and u is 0.

In some embodiments of (VIIIa) or (VIIIb), t is 3, s is 1, r is 2, and u is 1. In some embodiments of (VIIIa) or (VIIIb), t is 3, s is 1, r is 2, and u is 0.

In some embodiments of (VIIIa) or (VIIIb), t is 3, s is 1, r is 3, and u is 1. In some embodiments of (VIIIa) or (VIIIb), t is 3, s is 1, r is 3, and u is 0.

In some embodiments of (VIIIa) or (VIIIb), t is 3, s is 2, r is 1, and u is 1. In some embodiments of (VIIIa) or (VIIIb), t is 3, s is 2, r is 1, and u is 0.

In some embodiments of (VIIIa) or (VIIIb), t is 3, s is 2, r is 2, and u is 1. In some embodiments of (VIIIa) or (VIIIb), t is 3, s is 2, r is 2, and u is 0.

In some embodiments of (VIIIa) or (VIIIb), t is 3, s is 2, r is 3, and u is 1. In some embodiments of (VIIIa) or (VIIIb), t is 3, s is 2, r is 3, and u is 0.

In some embodiments of (VIIIa) or (VIIIb), t is 3, s is 3, r is 1, and u is 1. In some embodiments of (VIIIa) or (VIIIb), t is 3, s is 3, r is 1, and u is 0.

In some embodiments of (VIIIa) or (VIIIb), t is 3, s is 3, r is 2, and u is 1. In some embodiments of (VIIIa) or (VIIIb), t is 3, s is 3, r is 2, and u is 0.

In some embodiments of (VIIIa) or (VIIIb), t is 3, s is 3, r is 3, and u is 1. In some embodiments of (VIIIa) or (VIIIb), t is 3, s is 3, r is 3, and u is 0.

### 4.3.1. Exemplary Linkers and Linking Moieties

Exemplary linkers and linking moieties that can be utilized in the preparation of compounds of this disclosure (e.g., that link the M6PR binding moiety (X) to the moiety of interest (Y) in formula (XI)-(XV)) are shown in Tables 4-6.

In certain embodiments, the linker includes a linear linker or linking moiety as shown in Table 4. In certain embodiments, the linker includes a linear linker or linking moiety as shown in Table 5. In certain embodiments, the linker includes a linear linker or linking moiety as shown in Table 6. It is understood that various terminal modifications to the exemplary linking moieties can be incorporated based on the synthetic procedure and/or conjugation chemistries utilized in the preparation of the compounds.

Table 4 shows a variety of example linkers or linking moieties that find use in the compounds described herein. In some embodiments of formula (XI)-(XV), the compound includes any one of the linkers or linking moieties set forth in Table 4.

TABLE 4
Exemplary linear linkers and linking moieties

| Linker | |
|---|---|
| No. | Linker structure |
| L1 | 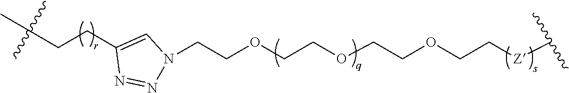 |
| | r is 0 to 10, q is 0 to 20, s is 0 or 1, Z′ is CO, NHCO, CONH or NH |
| L1.1 | 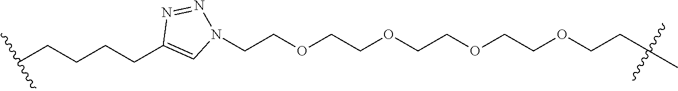 [SMILES: *CCCCc1cn(CCOCCOCCOCCOCC*)nn1] |
| L1.2 | 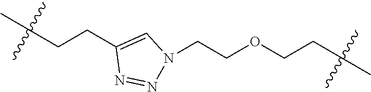 [SMILES: *CCOCCn1cc(CC*)nn1] |
| L1.3 | 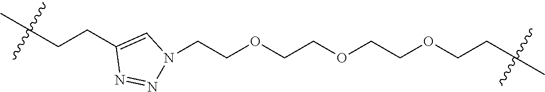 [SMILES: *CCOCCOCCOCCn1cc(CC*)nn1] |
| L1.4 | 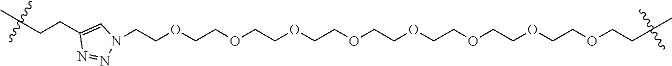 [SMILES: *CCOCCOCCOCCOCCOCCOCCOCCOCCn1cc(CC*)nn1] |
| L1.5 | 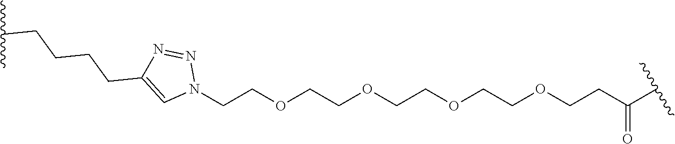 [SMILES: *CCCCc1cn(CCOCCOCCOCCOCCC(*)=O)nn1] |
| L1.6 | 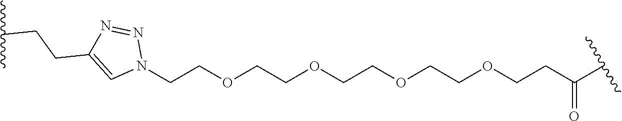 [SMILES: *CCc1cn(CCOCCOCCOCCOCCC(*)=O)nn1] |
| L1.7 | 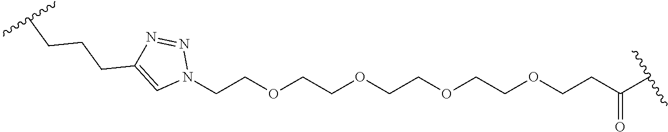 [SMILES: *CCCc1cn(CCOCCOCCOCCOCCC(*)=O)nn1] |
| L1.8 | 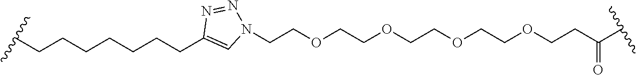 [SMILES: *CCCCCCCc1cn(CCOCCOCCOCCOCCC(*)=O)nn1] |
| L1.9 | 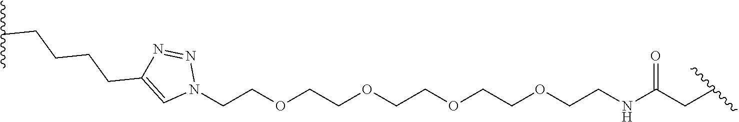 [SMILES: *CCCCc1cn(CCOCCOCCOCCOCCNC(=O)C*)nn1] |
| L1.10 | 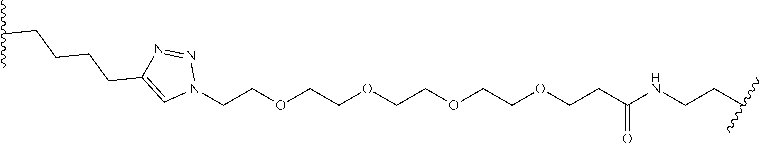 [SMILES: *CCCCc1cn(CCOCCOCCOCCOCCC(=O)NCC*)nn1] |
| L1.11 | 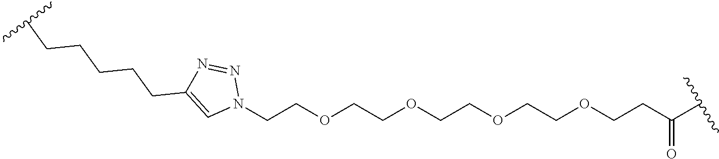 [SMILES: *CCCCCc1cn(CCOCCOCCOCCOCCC(*)=O)nn1] |
| L2 | 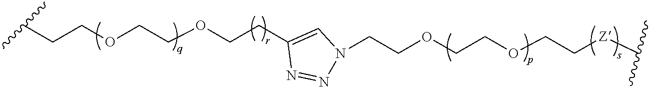 |
| | r is 0 to 10, p and q are 0 to 20, s is 0 or 1, Z′ is CO, NHCO, CONH or NH |
| L2.1 | 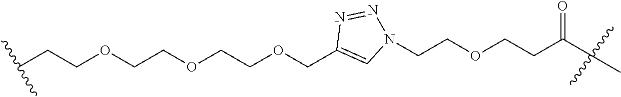 [SMILES: *CCOCCOCCOCc1cn(CCOCCC(*)=O)nn1] |
| L3 | 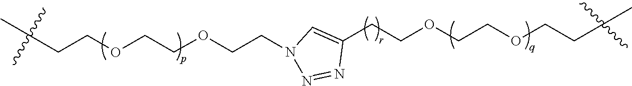 |
| | r is 0 to 10, p and q are independently 0 to 20 |
| L4 | 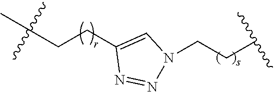 |
| | r is 0 to 10, s is 1 to 10 |
| L5 | 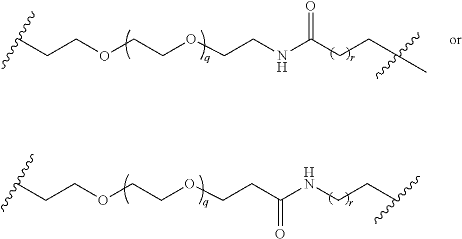 |
| | where r is 0 to 10, q is 0 to 20 |
| L5.1 | 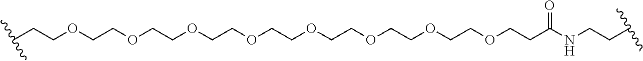 [SMILES: *CCNC(=O)CCOCCOCCOCCOCCOCCOCCOCCOCC*] |
| L6 | 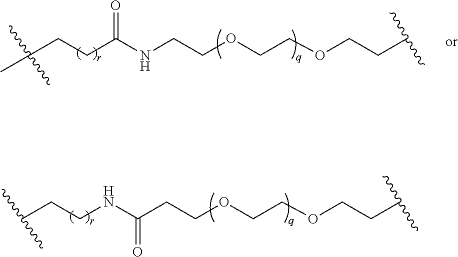 |
| | r is 0 to 10, q is 0 to 20 |
| L7 | 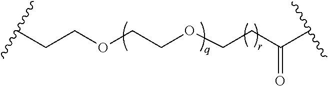 |
| | r is 0 to 10, q is 0 to 20 |
| L7.1 | 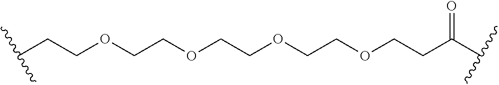 [SMILES: *CCOCCOCCOCCOCCC(*)=O] |
| L7.2 | 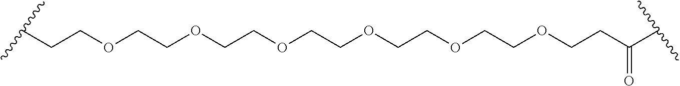 [SMILES: *CCOCCOCCOCCOCCOCCOCCC(*)=O] |
| L7.3 | 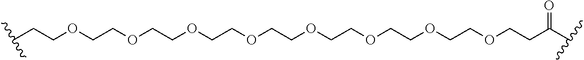 [SMILES: *CCOCCOCCOCCOCCOCCOCCOCCOCCC(*)=O] |
| L8 | 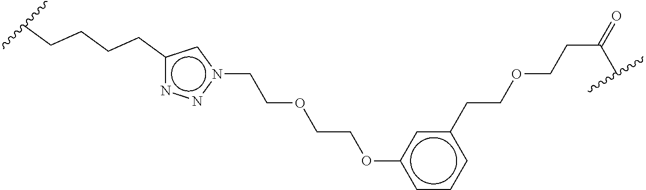 |
| L9 | 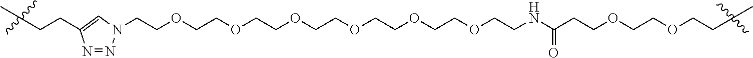 [SMILES: *CCOCCOCCC(=O)NCCOCCOCCOCCOCCOCCOCCn1cc(CC*)nn1] |
| L10 | 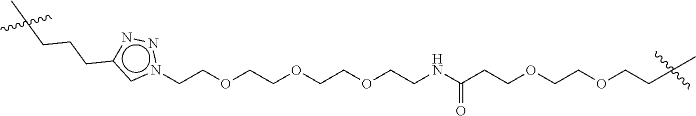 |
| L11 | 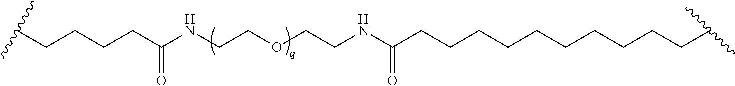 |
| | q is 0 to 10 |

TABLE 5
Exemplary branched linkers and branched linking moieties

| Linker | |
|---|---|
| No. | Linker structure |
| L21 | 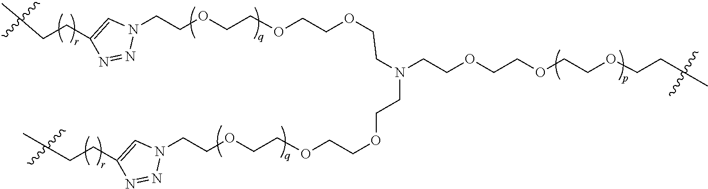 |
| | r is 0 to 10, q and p are independently 0 to 20 |
| L22 | 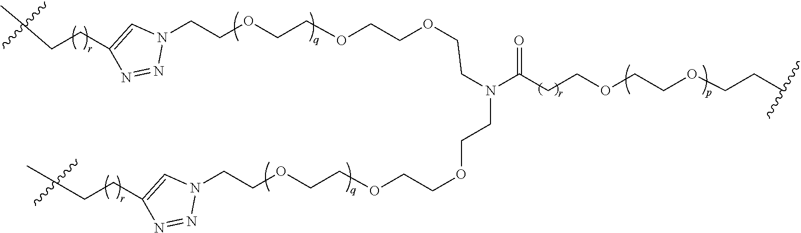 |
| | each r is independently 0 to 10, q and p are independently 0 to 20 |
| L23 | 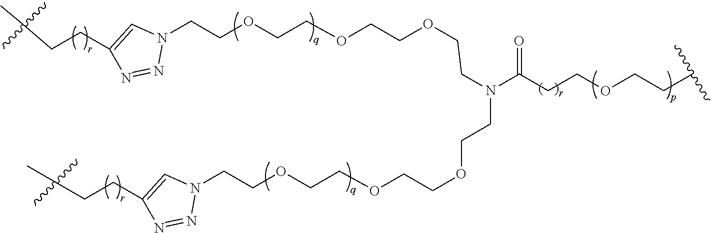 |
| | each r is independently 0 to 10, q and p are independently 0 to 20 |
| L24 | 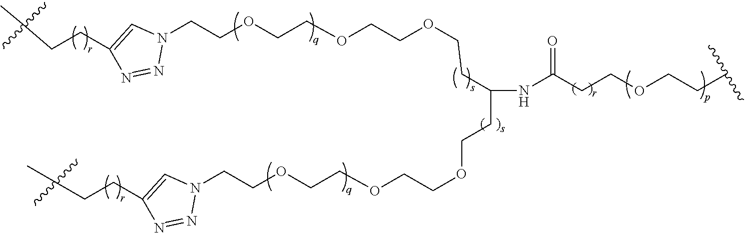 |
| | each r is independently 0 to 10, s is 0 or 1, q and p are independently 0 to 20 |
| L25 | 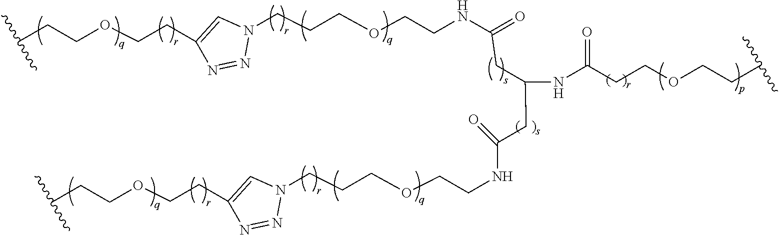 |
| | each r is independently 0 to 10, s is 0 or 1, each q and p is independently 0 to 20 |
| L26 | 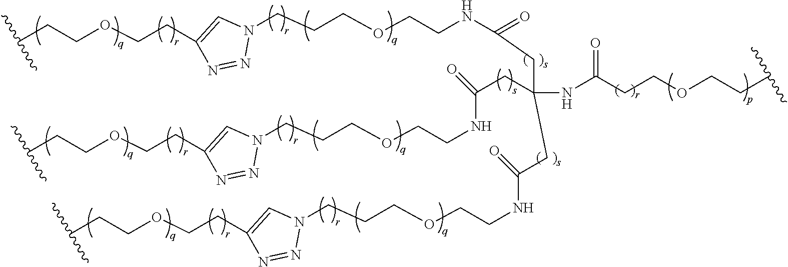 |
| | each r is independently 0 to 10, s is 0 or 1, each q and p is independently 0 to 20 |
| L26.1 | 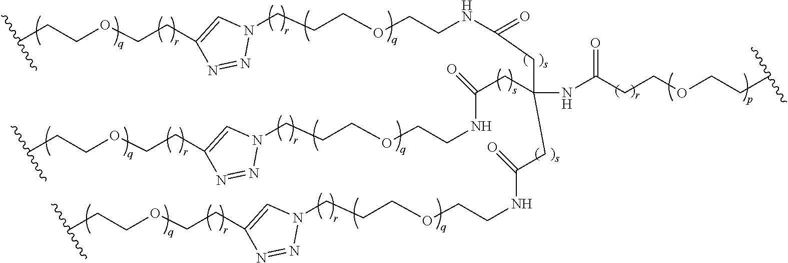 |
| | each r is independently 0 to 10, s is 0 or 1, each q and p is independently 0 to 20 |
| L27 | 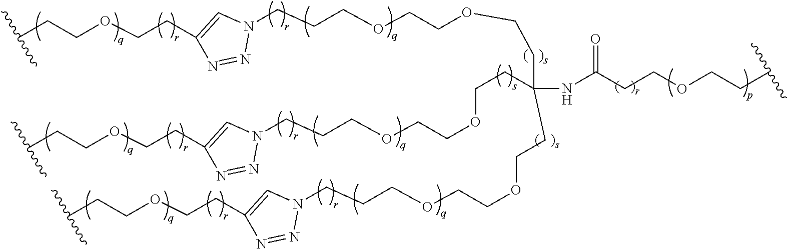 |
| | each r is independently 0 to 10, s is 0 or 1, each q and p is independently 0 to 20 |
| L28 | 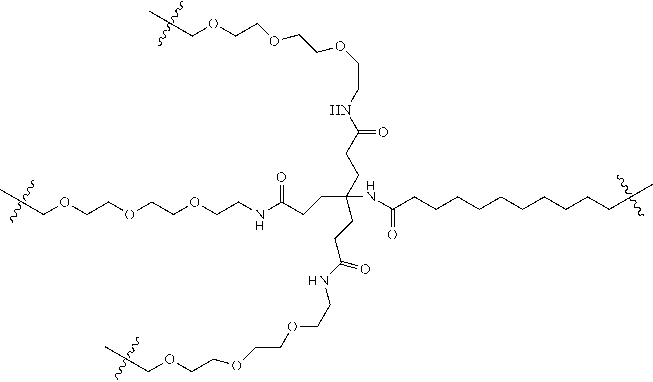 [SMILES: *CCCCCCCCCCC(=O)NC(CCC(=O)NCCOCCOCCOC*)(CCC(=O)NCCOCCOCCOC*)CCC(=O)NCCOCCOCCOC*] |
| L29 | 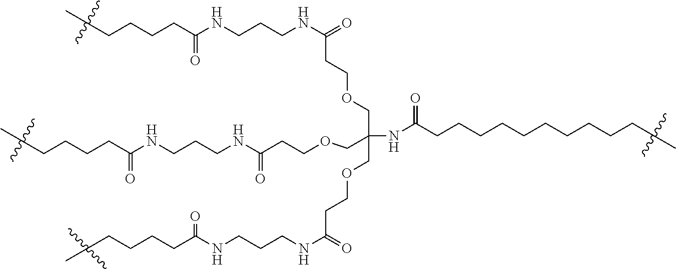 [SMILES: *CCCCCCCCCCC(=O)NC(COCCC(=O)NCCCNC(=O)CCCC*)(COCCC(=O)NCCCNC(=O)CCCC*)COCCC(=O)NCCCNC(=O)CCCC*] |
| L30 | 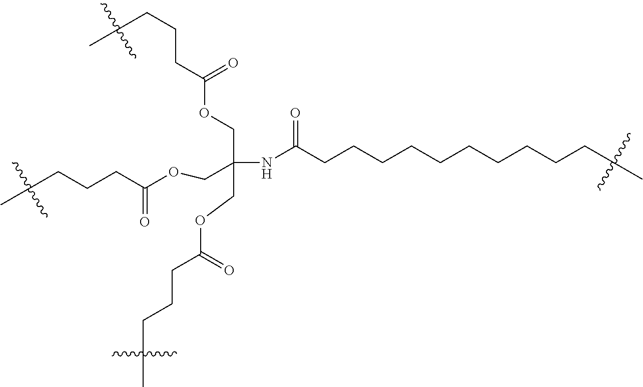 [SMILES: *CCCCCCCCCCC(=O)NC(COC(=O)CCC*)(COC(=O)CCC*)COC(=O)CCC*] |
| L31 | 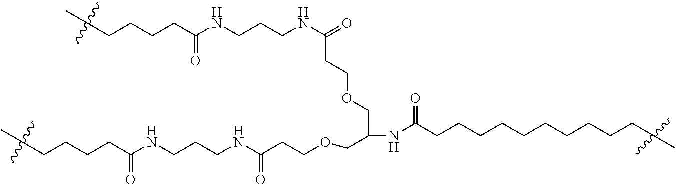 [SMILES: *CCCCCCCCCCC(=O)NC(COCCC(=O)NCCCNC(=O)CCCC*)COCCC(=O)NCCCNC(=O)CCCC*] |
| L32 | 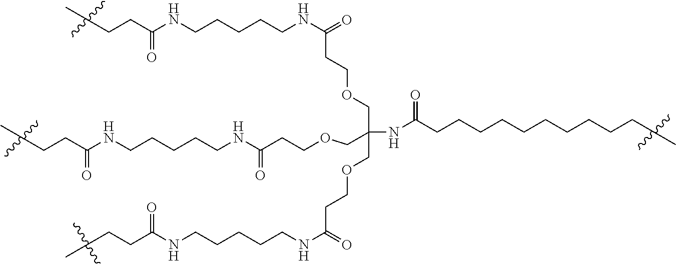 [SMILES: *CCCCCCCCCCC(=O)NC(COCCC(=O)NCCCCCNC(=O)CC*)(COCCC(=O)NCCCCCNC(=O)CC*)COCCC(=O)NCCCCCNC(=O)CC*] |
| L33 | 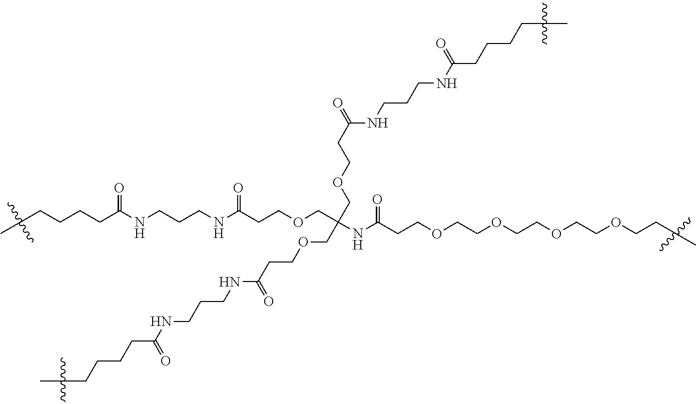 [SMILES: *CCCCC(=O)NCCCNC(=O)CCOCC(COCCC(=O)NCCCNC(=O)CCCC*)(COCCC(=O)NCCCNC(=O)CCCC*)NC(=O)CCOCCOCCOCCOCC*] |
| L34 | 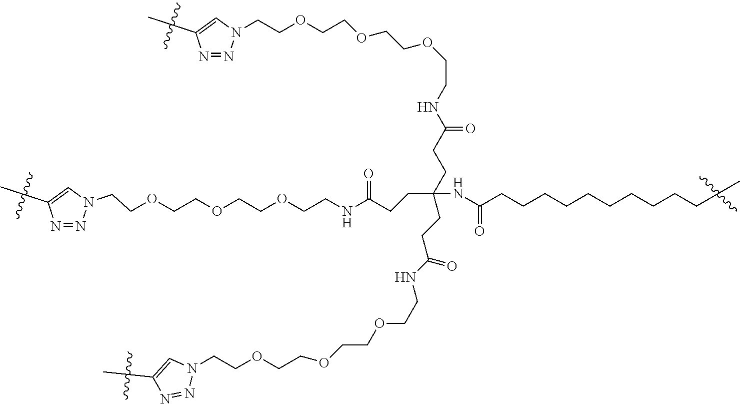 [SMILES: *CCCCCCCCCCC(=O)NC(CCC(=O)NCCOCCOCCOCCn1cc(*)nn1)(CCC(=O)NCCOCCOCCOCCn1cc(*)nn1)CCC(=O)NCCOCCOCCOCCn1cc(*)nn1] |
| L35 L36 | 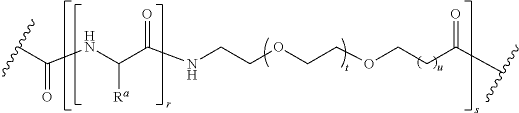 |
| | 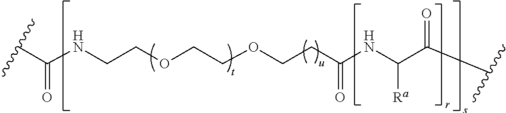 |
| | where r is 1-3, t is 3-5, u is 0 or 1, and s is 2-5 |

Table 6 illustrates exemplary synthetic precursors of linker components that are used to prepare compounds of this disclosure, e.g., via a conjugation chemistry. It is understood that a variety of homologs of the structures shown in Table 6 are also encompassed by this disclosure that provide for linkers of a variety of lengths. It is understood that alternative chemoselective ligation groups and other chemical functional groups can also be incorporated as needed to prepare a desired linker.

TABLE 6
Linker component synthetic precursors

| Re- | |
|---|---|
| agent | |
| # | Structure |
| LC1 | 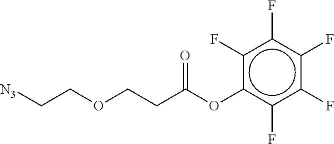 |
| LC2 | 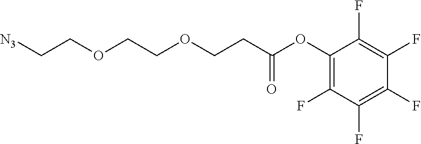 [SMILES: [N-]=[N+]=NCCOCCOCCC(=O)Oc1c(F)c(F)c(F)c(F)c1F] |
| LC3 | 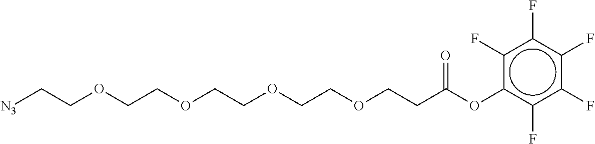 |
| LC4 | 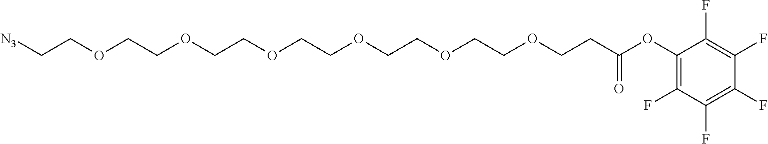 [SMILES: [N-]=[N+]=NCCOCCOCCOCCOCCOCCOCCC(=O)Oc1c(F)c(F)c(F)c(F)c1F] |
| LC5 | 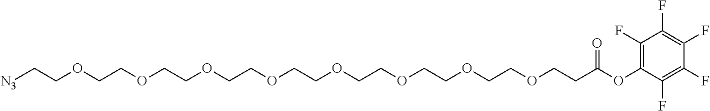 [SMILES: [N-]=[N+]=NCCOCCOCCOCCOCCOCCOCCOCCOCCC(=O)Oc1c(F)c(F)c(F)c(F)c1F] |
| LC6 | 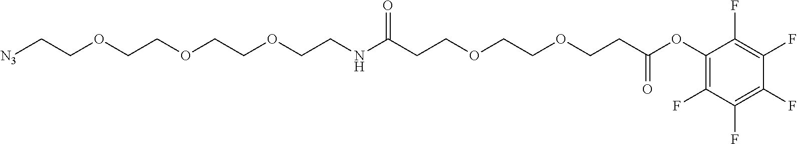 [SMILES: [N-]=[N+]=NCCOCCOCCOCCNC(=O)CCOCCOCCC(=O)Oc1c(F)c(F)c(F)c(F)c1F] |
| LC7 | 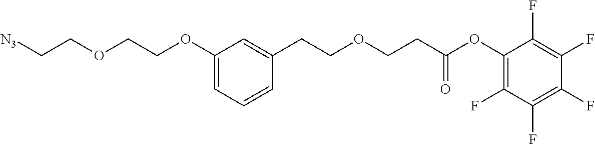 [SMILES: [N-]=[N+]=NCCOCCOc1cccc(CCOCCC(=O)Oc2c(F)c(F)c(F)c(F)c2F)c1] |
| LC8 | 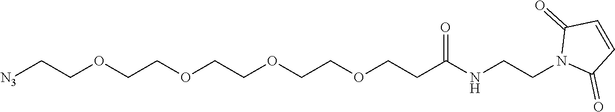 [SMILES: [N-]=[N+]=NCCOCCOCCOCCOCCC(=O)NCCN1C(=O)C=CC1=O] |
| LC9 | 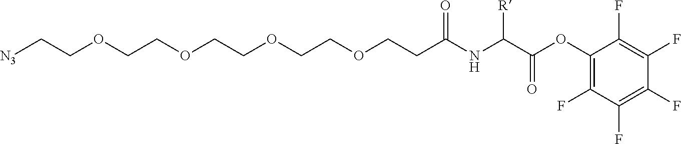 |
| LC10 | 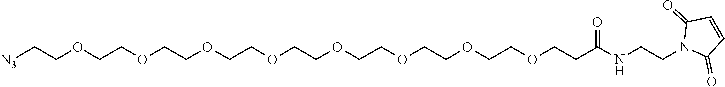 [SMILES: [N-]=[N+]=NCCOCCOCCOCCOCCOCCOCCOCCOCCC(=O)NCCN1C(=O)C=CC1=O] |
| LC11 | 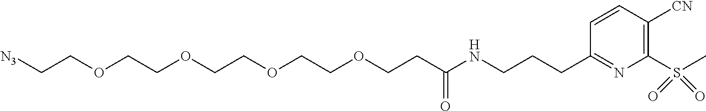 [SMILES: CS(=O)(=O)c1nc(CCCNC(=O)CCOCCOCCOCCOCCN=[N+]=[N-])ccc1C#N] |
| LC12 | 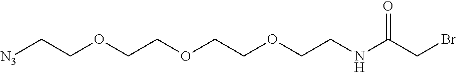 [SMILES: [N-]=[N+]=NCCOCCOCCOCCNC(=O)CBr] |
| LC13 | 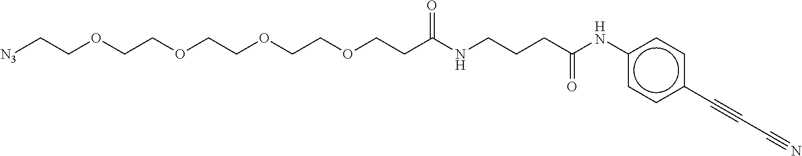 |
| LC14 | 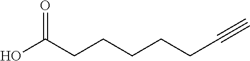 |
| LC15 | 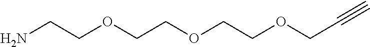 |
| LC16 | 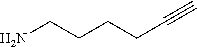 |
| LC17 | 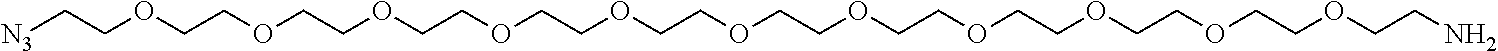 [SMILES: [N-]=[N+]=NCCOCCOCCOCCOCCOCCOCCOCCOCCOCCOCCOCCN] |
| LC18 | 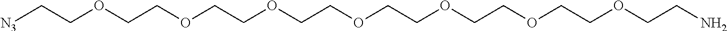 [SMILES: [N-]=[N+]=NCCOCCOCCOCCOCCOCCOCCOCCN] |
| LC19 | 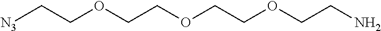 [SMILES: [N-]=[N+]=NCCOCCOCCOCCN] |
| LC20 | 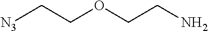 |
| LC21 | 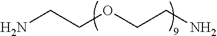 |
| LC22 | 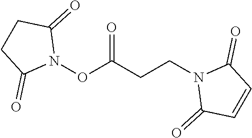 [SMILES: O=C(CCN1C(=O)C=CC1=O)ON1C(=O)CCC1=O] |
| LC23 | 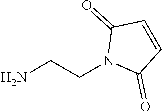 |
| LC24 | 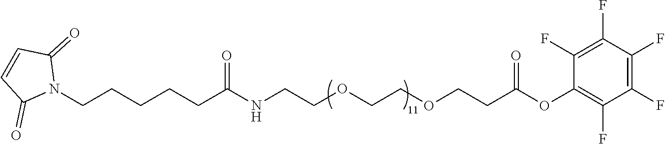 |
| LC25 | 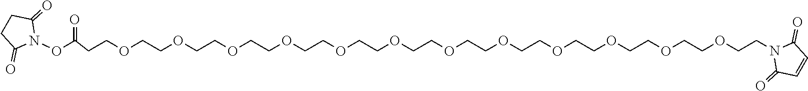 [SMILES: O=C(CCOCCOCCOCCOCCOCCOCCOCCOCCOCCOCCOCCOCCN1C(=O)C=CC1=O)ON1C(=O)CCC1=O] |
| LC26 | 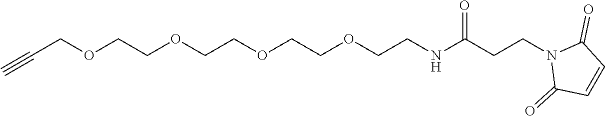 [SMILES: C#CCOCCOCCOCCOCCNC(=O)CCN1C(=O)C=CC1=O] |
| LC27 | 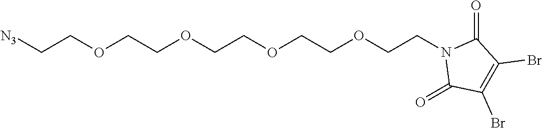 [SMILES: [N-]=[N+]=NCCOCCOCCOCCOCCN1C(=O)C(Br)=C(Br)C1=O] |
| LC28 | 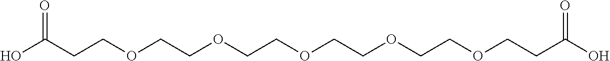 [SMILES: O=C(O)CCOCCOCCOCCOCCOCCC(=O)O] |
| LC29 | 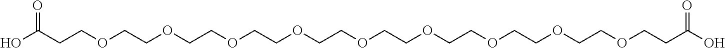 [SMILES: O=C(O)CCOCCOCCOCCOCCOCCOCCOCCOCCOCCC(=O)O] |
| LC30 | 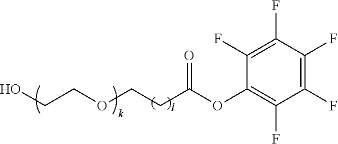 |
| | k = 4, l = 0 |
| | k = 0, l = 12 |
| | k = 2, l = 6 |
| LC31 | 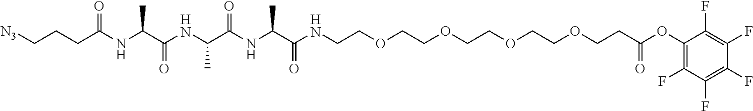 [SMILES: C[C@H](NC(=O)CCCN=[N+]=[N-])C(=O)N[C@@H](C)C(=O)N[C@@H](C)C(=O)NCCOCCOCCOCCOCCC(=O)Oc1c(F)c(F)c(F)c(F)c1F] |
| LC32 | 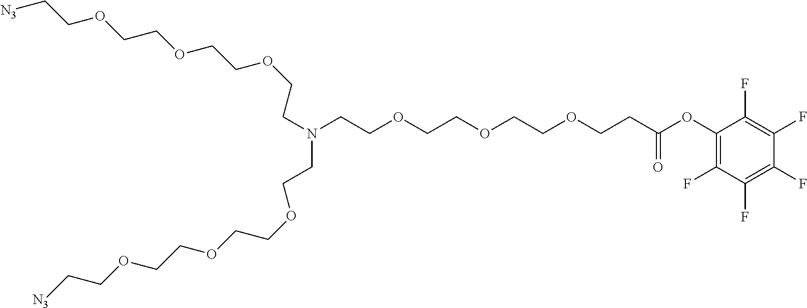 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F] |
| LC33 | 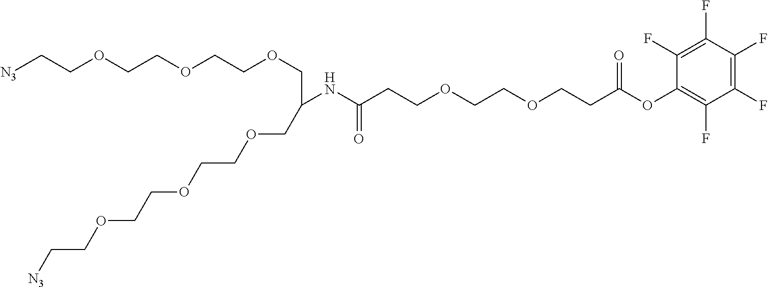 [SMILES: [N-]=[N+]=NCCOCCOCCOCC(COCCOCCOCCN=[N+]=[N-])NC(=O)CCOCCOCCC(=O)Oc1c(F)c(F)c(F)c(F)c1F] |
| LC34 | 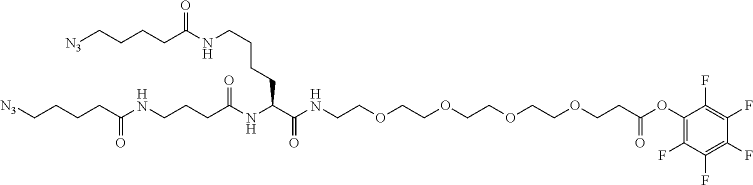 [SMILES: [N-]=[N+]=NCCCCC(=O)NCCCC[C@H](NC(=O)CCCNC(=O)CCCCN=[N+]=[N-])C(=O)NCCOCCOCCOCCOCCC(=O)Oc1c(F)c(F)c(F)c(F)c1F] |
| LC35 | 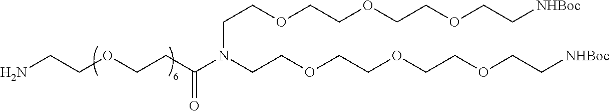 |
| LC36 | 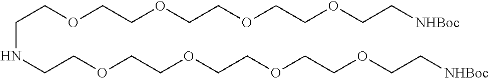 [SMILES: CC(C)(C)OC(=O)NCCOCCOCCOCCOCCNCCOCCOCCOCCOCCNC(=O)OC(C)(C)C] |
| LC37 | 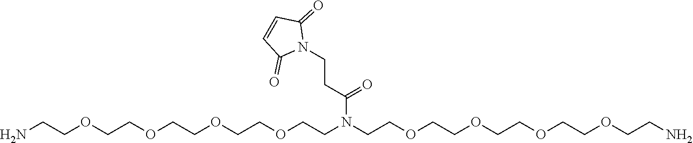 [SMILES: NCCOCCOCCOCCOCCN(CCOCCOCCOCCOCCN)C(=O)CCN1C(=O)C=CC1=O] |
| LC38 | 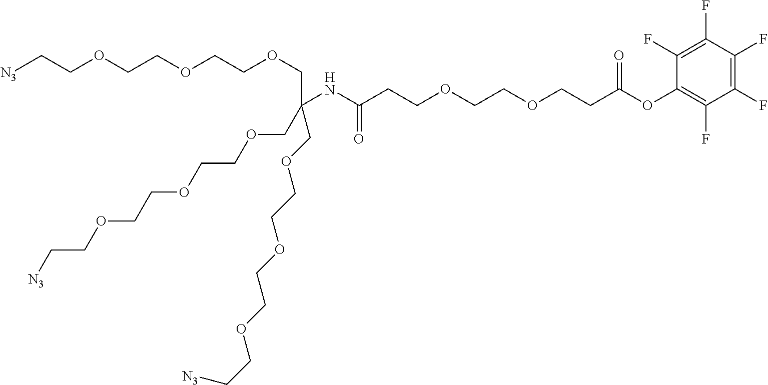 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F] |
| LC39 | 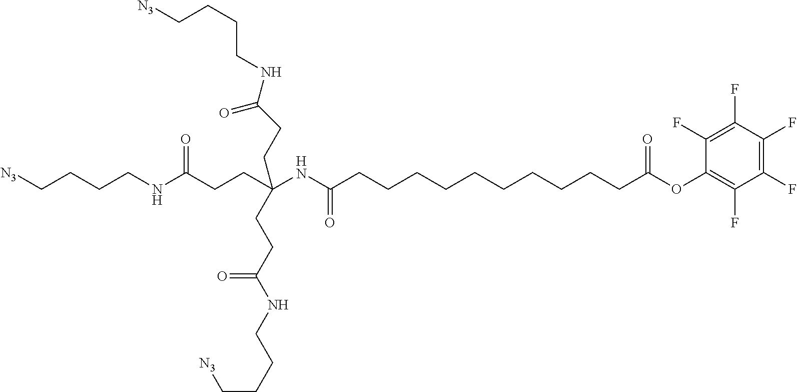 [SMILES: [N-]=[N+]=NCCCCNC(=O)CCC(CCC(=O)NCCCCN=[N+]=[N-])(CCC(=O)NCCCCN=[N+]=[N-])NC(=O)CCCCCCCCCCC(=O)Oc1c(F)c(F)c(F)c(F)c1F] |
| LC40 | 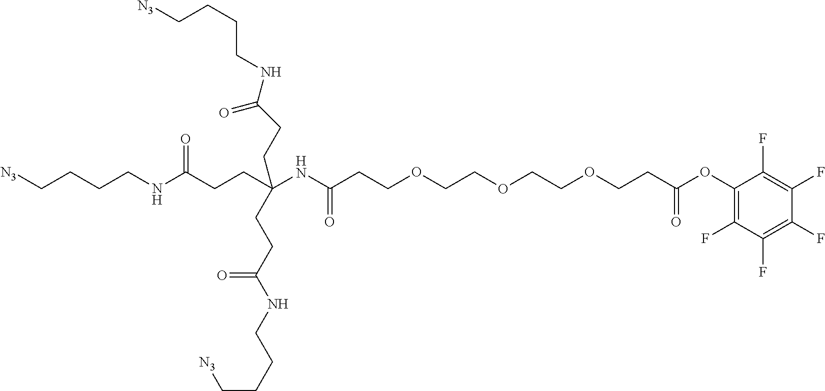 [SMILES: [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)Oc1c(F)c(F)c(F)c(F)c1F] |
| LC41 | 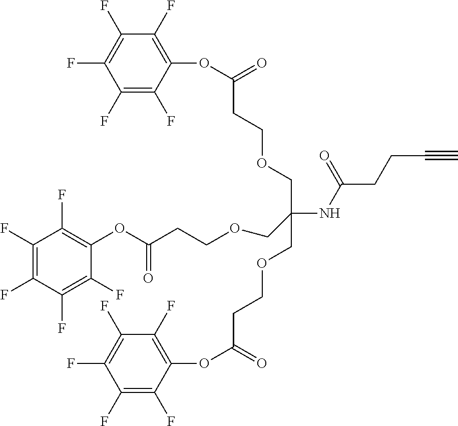 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F] |
| LC42 | 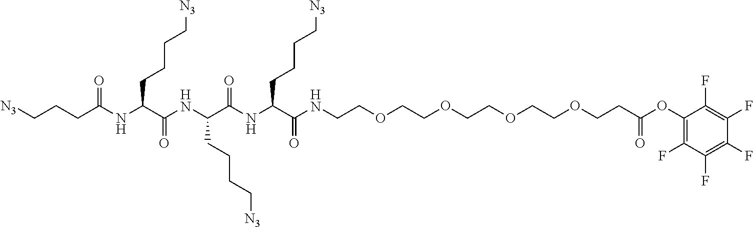 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC43 | 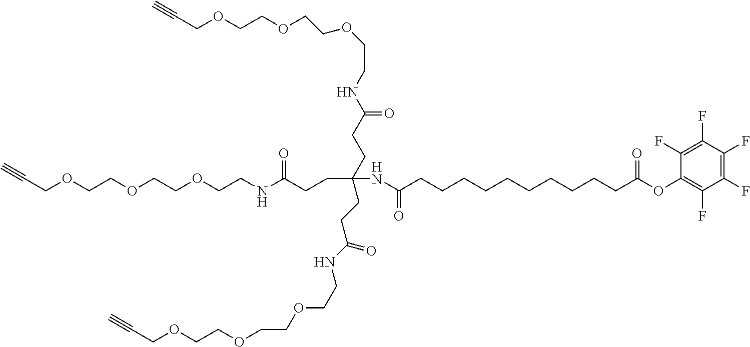 [SMILES: C#CCOCCOCCOCCNC(=O)CCC(CCC(=O)NCCOCCOCCOCC#C)(CCC(=O)NCCOCCOCCOCC#C)NC(=O)CCCCCCCCCCC(=O)Oc1c(F)c(F)c(F)c(F)c1F] |
| LC44 | 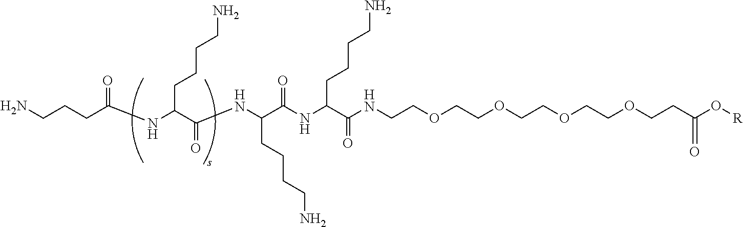 |
| | where R is H, or protecting group, and s is 1, 2, 3, 5-10, or 10-100, or 20-50. |
| LC45 | 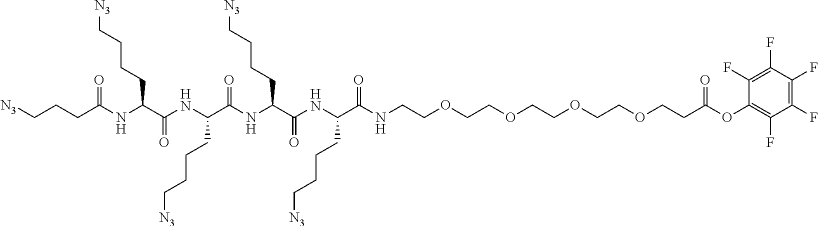 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC46 | 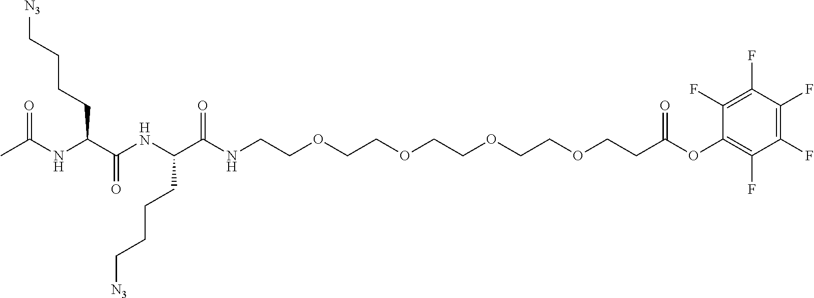 [SMILES: CC(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC47 | 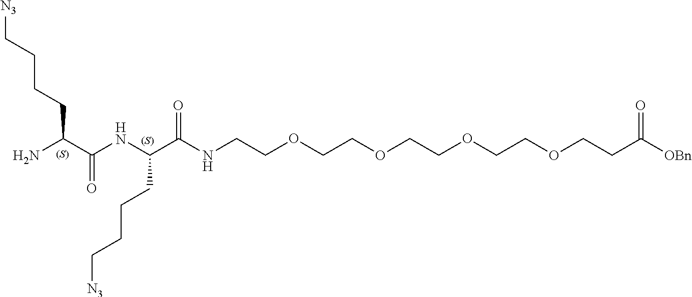 |
| LC48 | 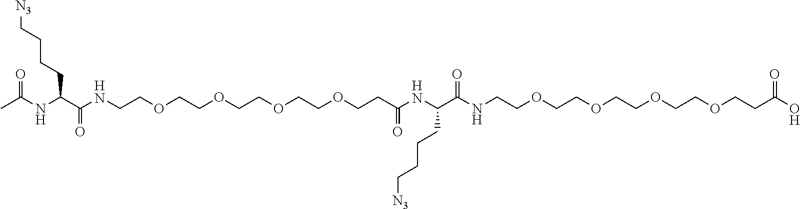 [SMILES: CC(=O)N[C@@H](CCCCN=[N+]=[N-])C(=O)NCCOCCOCCOCCOCCC(=O)N[C@@H](CCCCN=[N+]=[N-])C(=O)NCCOCCOCCOCCOCCC(=O)O] |
| LC49 LC50 LC51 LC52 LC53 | 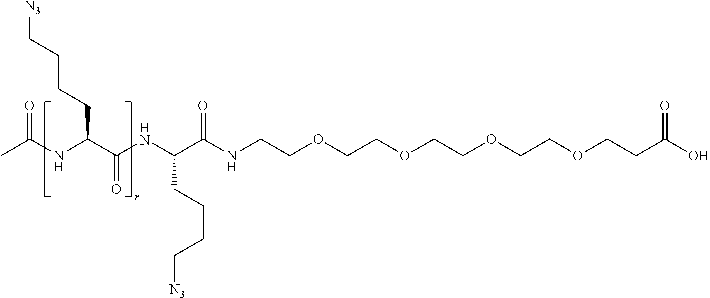 |
| | LC49 where r is 0 |
| | LC50 where r is 1 |
| | LC51 where r is 2 |
| | LC52 where r is 3 |
| | LC53 where r is 4 |
| LC54 LC55 LC56 LC57 LC58 LC59 LC60 | 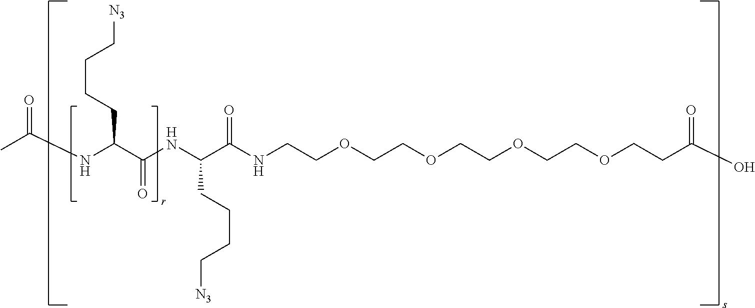 |
| | LC54 where r is 0, s is 2 |
| | LC55 where r is 1, s is 2 |
| | LC56 where r is 2, s is 2 |
| | LC57 where r is 0, s is 3 |
| | LC58 where r is 1, s is 3 |
| | LC59 where r is 2, s is 3 |
| | LC60 where r is 0-4, s is 4-20 |
| L61 L62 | 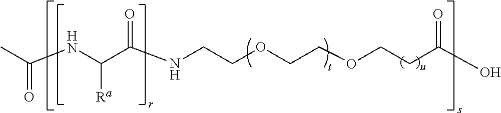 |
| | 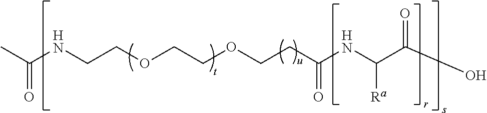 |
| | where r is 1-3, t is 3-5, u is 0 or 1, and s is 2-5 |

### 4.4. Chemoselective Ligation Group

In certain embodiments of formula (XI)-(XV), Y is a chemoselective ligation group, or a precursor thereof. A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, tyrosine specific conjugation chemistry (e.g., e-Y-CLICK), methionine specific conjugation chemistry (e.g., oxaziridine-based or ReACT chemistry), reductive amination, dialkyl squarate chemistry, etc.

Table 6 illustrates exemplary synthetic precursors of linker components that are used to prepare compounds of this disclosure, and which have various chemoselective ligation groups. A variety of other chemical functional groups can also be incorporated as needed to prepare a desired linker.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments, Y is a reactive moiety capable forming a covalent bond to a polypeptide (e.g., with an amino acid sidechain of a polypeptide having a compatible reactive group). The reactive moiety can be referred to as a chemoselective ligation group.

Example chemoselective ligation groups, and synthetic precursors thereof, which may be adapted for use in the compounds of this disclosure are shown in Table 6B.

TABLE 6B
Exemplary chemoselective ligation groups and precursors

| Groups | Exemplary structures | |
|---|---|---|
| carboxylic acid or active ester | 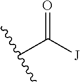 | |
| | where J is selected from | |
| | —OH, —Cl, —Br, —I, —F, —OH, | |
| | —O—N-succinimide, | |
| | —O-(4-nitrophenyl), —O-pentafluorophenyl, | |
| | —O-tetrafluorophenyl, and | |
| | —O—C(O)—OR^{J′}, and R^{J′} is —C₁-C₈ alkyl or -aryl, | |
| | 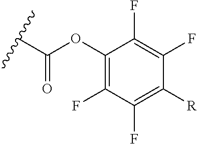 | |
| | R is H or F, | |
| | 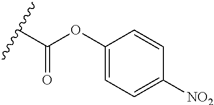 [SMILES: *C(=O)Oc1ccc([N+](=O)[O-])cc1] | 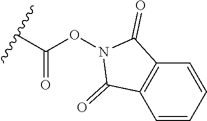 [SMILES: *C(=O)ON1C(=O)c2ccccc2C1=O] |
| | 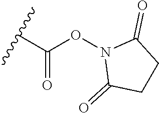 [SMILES: *C(=O)ON1C(=O)CCC1=O] | 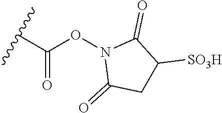 [SMILES: *C(=O)ON1C(=O)CC(S(=O)(=O)O)C1=O] |
| | 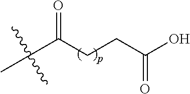 | 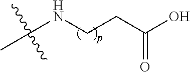 |
| | where p is 0 to 6 | |
| maleimide | 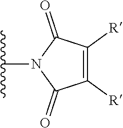 | |
| | where each R′ is independently hydrogen or halogen (e.g., bromo) | |
| isocyanate or | —NCS | |
| isothiocyanate | —NCO | |
| | 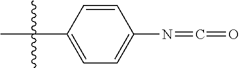 [SMILES: *c1ccc(N=C=O)cc1] | |
| | 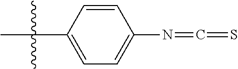 [SMILES: *c1ccc(N=C=S)cc1] | |
| alkyl halide alkyl tosylate | 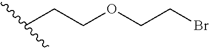 [SMILES: *CCOCCBr] | |
| | 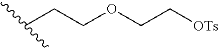 [SMILES: *CCOCCOS(=O)(=O)c1ccc(C)cc1] | |
| aldehyde | 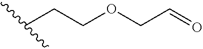 [SMILES: *CCOCC=O] | |
| haloacetamide or alpha-leaving group acetamide | 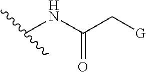 | |
| | where G is selected from —Cl, —Br, —I, —O-mesyl, and —O-tosyl | |
| | 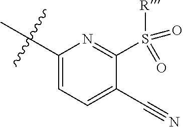 | |
| | where R′′′ is alkyl | |
| diazirine | 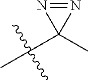 [SMILES: *C1(C)N=N1] | |
| sulfonyl halide or vinyl sulfone | 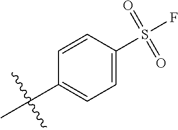 [SMILES: *c1ccc(S(=O)(=O)F)cc1] | 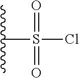 [SMILES: *S(=O)(=O)Cl] |
| | 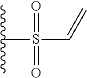 [SMILES: *S(=O)(=O)C=C] | |
| hydrazide hydrazino hydroxylamino | 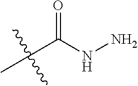 [SMILES: *C(=O)NN] | 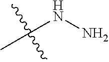 [SMILES: *NN] |
| | 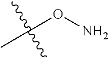 [SMILES: *ON] | |
| pyridyl disulfide | 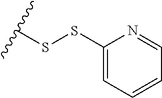 [SMILES: *SSc1ccccn1] | |
| (HIPS hydrazinyl- indolyl group, or (aza-HIPS) hydrazinyl- pyrrolo- pyridinyl group | 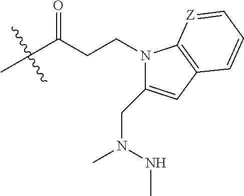 | |
| | where Z is CH or N | |
| alkyne or cyclooctyne | 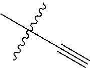 [SMILES: *C#C] | 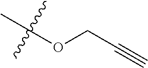 [SMILES: *OCC#C] |
| | 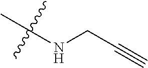 [SMILES: *NCC#C] | 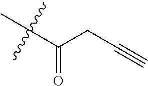 [SMILES: *C(=O)CC#C] |
| | 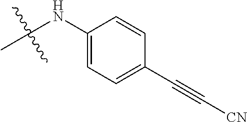 [SMILES: *Nc1ccc(C#CC#N)cc1] | |
| | 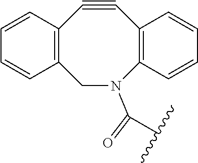 [SMILES: *C(=O)N1Cc2ccccc2C#Cc2ccccc21] | 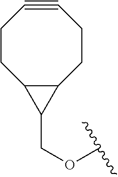 [SMILES: *OCC1C2CCC#CCCC21] |
| azide | 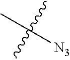 [SMILES: *N=[N+]=[N-]] | 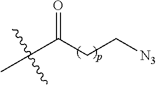 |
| | 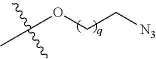 | 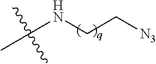 |
| | where p is 0 to 6 and where q is 1 to 6 | |
| amine | 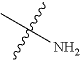 [SMILES: *N] | 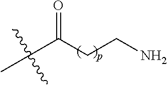 |
| | 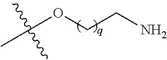 | 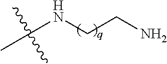 |

where p is 0 to 6 and where q is 1 to 6

In Table 6B, the [symbol] can represent a point of attachment of Y to a linking moiety or a linked X moiety (e.g., M6PR binding moiety).

### 4.5. Conjugates

Aspects of this disclosure include conjugates of the compounds described herein, e.g., of formula (XIII), where Y is a chemoselective ligation group, with another moiety of interest. When such a conjugate is prepared, one or multiple M6PR ligand-linker compounds can be attached or conjugated to another moiety of interest. For example, when the moiety of interest is a biomolecule, the chemoselective ligation group of a M6PR ligand-linker compound can be conjugated at one or several sites of the biomolecule. It is understood that such biomolecule conjugates of this disclosure can be encompassed by Formula (XI), (XII) and (II)-(III), and by the formula described below.

In some embodiments, a conjugate of this disclosure is described by formula (XII):

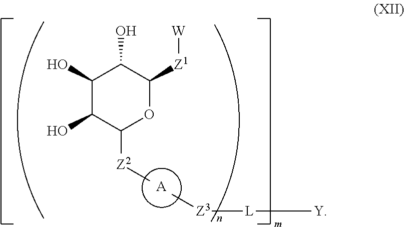

or a prodrug thereof, or a salt thereof (e.g., a pharmaceutically acceptable salt), wherein:
W is a non-hydrolyzable hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁—C)alkyl, and each R²² is independently selected from H, halogen (e.g., F) and optionally substituted (C₁—C)alkyl;
each A is independently a cyclic group (e.g., an optionally substituted aryl or heteroaryl linking moiety);
each Z³ is independently a linking moiety;
n is 1 to 500;
m is 1 to 100;
L is a linker; and
Y is a biomolecule.

In some embodiments of formula (XII), when A is phenyl and Z² is O, then:
(i) W is —P(O)(OH)₂; or
(ii) the linker L comprises a backbone of at least 16 consecutive atoms and Y is a target binding moiety.

In some embodiments of formula (XII), the cell surface mannose-6-phosphate receptor (M6PR) binding conjugate is of formula (XIIa):

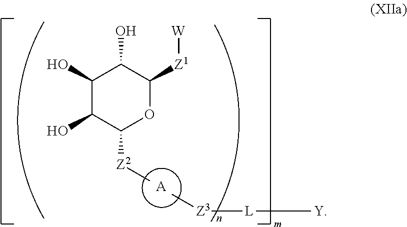

In some embodiments of formula (XII), the cell surface mannose-6-phosphate receptor (M6PR) binding conjugate is of formula (XIIa):

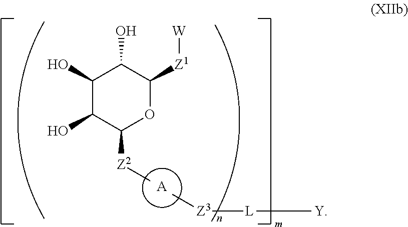

In some embodiments, the moiety of interest to which the M6PR binding moiety is linked is a biomolecule. In some embodiments, the moiety of interest is a biomolecule. In some embodiments, the biomolecule is selected from polypeptide (e.g., peptide or protein), polynucleotide, polysaccharide, glycan, glycoprotein, lipid, enzyme, antibody, and antibody fragment.

In some embodiments, the moiety of interest Y is selected from small molecule, small molecule drug, chemotherapeutic agent, cytotoxic agent, diagnostic agent, dye, fluorophore, and the like. In some embodiments, m is 1 where one M6PR binding moiety is linked to Y.

In some embodiments, one Y biomolecule is conjugated to a single moiety (X) that specifically binds to the cell surface M6PR via a linker L. In some embodiments, one Y biomolecule is conjugated to one (Xₙ-L)-group, wherein when n=1 the (Xₙ-L)-group is referred to as monovalent, and when n>1 the (Xₙ-L)-group is referred to as multivalent (e.g., bivalent, trivalent, etc.). It is understood that in some embodiments of the formula described herein, where Y is a biomolecule, Y can be conjugated to two or more (Xₙ-L)-groups, wherein each (Xₙ-L)-group may itself be monovalent or multivalent (e.g., bivalent, trivalent, etc.). In such cases, the ratio of linked (Xr-L)-groups to biomolecule can be referred to as 2 or more.

In some embodiments of formula (XII), the conjugate is produced from the conjugation of a compound of formula (XIII) where Y is chemoselective ligation group with a biomolecule, where the conjugate is of formula (XXI):

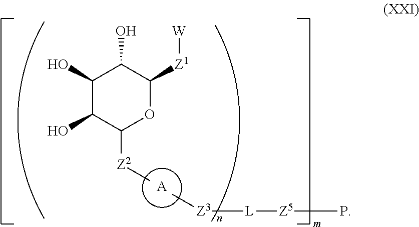

or a prodrug thereof, or pharmaceutically acceptable salt thereof, wherein:
n is 1 to 3;
m is a loading of 1 to 20;
L is a linker;
P is a biomolecule that specifically binds the target protein;
Z⁵ is a residual linking moiety resulting from the covalent linkage of a chemoselective ligation group located at the terminal of a linker of formula (XIII) to a compatible group of P. In some embodiments of formula (XXI), Z² is connected to the anomeric position of the pyranose ring with a beta configuration. Depending on the chemoselective ligation group and conjugation chemistry used, m can be an average loading (also referred to herein as DAR), or m can be a specific loading (e.g., m is 1 or 2).

In some embodiments of formula (XXI), the conjugate is of formula (XXIa):

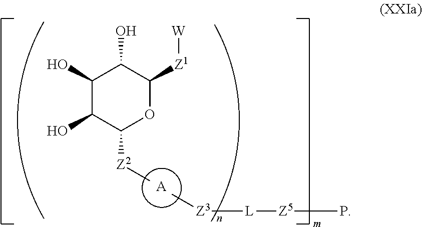

In some embodiments of formula (XXI), the conjugate is of formula (XXIa):

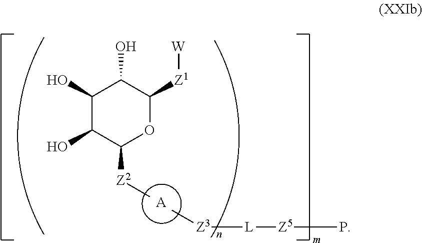

In some embodiments of formula (XXI)-(XXIb), n is 1.

In some embodiments of formula (XXI)-(XXIb), n is 2.

In some embodiments of formula (XXI)-(XXIb), n is 3.

In some embodiments of formula (XXI)-(XXIb), n is 4.

In some embodiments of formula (XXI)-(XXIb), n is 5 or more, such as n is 5 to 500, 5 to 100, 5 to 50, 5 to 20, or 5 to 10. In some embodiments of formula (XXI)-(XXIb), n is 5. In some embodiments of formula (XXI)-(XXIb), n is 10 to 100, such as 10-50, 10-20 or 20-50. In some embodiments of formula (XXI)-(XXIb), L includes a polypeptide, such as a polylysine, or polyserine derivative. In some embodiments of formula (XXI)-(XXIb), L is a polypeptide containing linker where one M6PR binding moiety (X) is attached to L per amino acid residue of the polypeptide.

In some embodiments of formula (XXI)-(XXIb), m is the average loading of the M6PR binding moiety (X) on biomolecule P. For example, when a lysine conjugation chemistry is used to link X to P, and P includes multiple lysine residues, it is understood that m can refer to an average loading.

In some embodiments of formula (XXI)-(XXIb), m is 1 to 10, such as 1 to 8, 1 to 7, or 1 to 6. In some embodiments of formula (XXI)-(XXIb), m is 2 to 20, such as 2 to 10, 2 to 8, 2 to 7, or 2 to 6. In some embodiments of formula (XXI)-(XXIb), m is at least 3. In some embodiments of formula (XXI)-(XXIb), m is at least 4.

In some embodiments of formula (XXI)-(XXIb), m is about 8, about 7, about 6, about 5, about 4, about 3 or about 2.

In some embodiments of formula (XXI)-(XXIb), n is 1, and m is 1 to 10. In some embodiments of formula (XXI)-(XXIb), m is 2 to 8 (e.g., 2 to 6, or 3 to 5). In some embodiments of formula (XXI)-(XXIb), m is about 4.

In some embodiments of formula (XXI)-(XXIb), m is a particular loading of the M6PR binding moiety (X) on biomolecule P. For example, when a site-specific conjugation chemistry is used to link X to P via the linker, it is understood that m can refer to a particular loading. In some embodiments of formula (XXI)-(XXIb), m is 1. In some embodiments, the biomolecule P is a polypeptide having a single site for conjugation. In some embodiments of formula (XXI)-(XXIb), m is 2. In some embodiments, the biomolecule P is an antibody. In some embodiments, the biomolecule P is an antibody fragment.

In some embodiments of formula (XXI)-(XXIb), n is 2, and m is 1 to 6 (e.g., 2 to 6, or 3 to 5). In some embodiments of formula (XXI)-(XXIb), m is about 4.

In some embodiments of formula (XXI)-(XXIb), n is 3, and m is 1 to 6 (e.g., 2 to 6, or 3 to 5).

In some embodiments of formula (XXI)-(XXIb), Z⁵ is a residual moiety resulting from the covalent linkage of a thiol-reactive chemoselective ligation group (e.g., maleimide) to one or more cysteine residue(s) of P, e.g.,

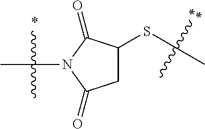

wherein [symbol] represents the point of attachment to the linker L, and [symbol] represents the point of attachment to P.

In some embodiments of formula (XXI)-(XXIb), Z⁵ is a residual moiety resulting from the covalent linkage of an amine-reactive chemoselective ligation group (e.g., PFP ester or TFP ester or NHS ester) to one or more lysine residue(s) of P, i.e., and amide bond —CONH—.

Additional residual moieties Z⁵ and chemoselective ligation groups from which they derive are described herein.

In some embodiments of formula (XXI)-(XXIb), L is a linear linker having a backbone of 16 or more consecutive atoms covalently linking Z³ to P (e.g., a backbone of 16-100, 18-100, or 20-100 consecutive atoms). In some embodiments of formula (XXI)-(XXIb), L is a branched linker having a backbone of 14 or more consecutive atoms (e.g., such as 14 to 50, or 14 to 30 atoms) between Z³ and the branching atom of the linker.

### 4.5.1. Target-Binding Moieties

In preferred embodiments, the moiety of interest is a molecule that specifically binds to a target of interest, i.e., a target-binding moiety. Accordingly, the compound of this disclosure can be referred to as a target protein degrading compound or conjugate. In such cases, the conjugates of this disclosure can provide for cellular uptake of the target after it non-covalently binds to the conjugate, followed by lysosomal degradation. The inventors have demonstrated that conjugates of this disclosure having a particular M6PR binding moieties of a desired affinity, with a linker of desired valency and length, can specifically bind with high affinity to both the M6PR and the target simultaneously. The conjugates of this disclosure can thus provide for internalization and sequestering of a bound target protein in the cell's lysosome and subsequent degrading of the target protein.

The target-binding moiety can be any moiety that has an affinity for the target of less than 1 μM, such as 300 nM or less, 100 nM or less, 30 nM or less, 10 nM or less, 3 nM or less, or 1 nM or less, e.g., as measured in an in vitro binding assay. In some embodiments, the target-binding moiety has an affinity of 10 nM or less, such as 1 nM or less for the target protein.

In some embodiments, the target-binding moiety is a biomolecule. In some embodiments, the target-binding moiety is a biomolecule that specifically binds to a target protein. In some embodiments, the biomolecule is selected from polypeptide (e.g., peptide or protein), polynucleotide, polysaccharide, glycan, antibody, antibody fragment, and glycoprotein. It is understood that the term polypeptide encompasses antibody, antibody fragment, and glycoprotein.

In some embodiments, the target-binding moiety is a polynucleotide that specifically binds to a target molecule, such as a target protein or a target nucleic acid. The terms polynucleotide and nucleic acid can be used interchangeably. In some embodiments, the target-binding moiety is a nucleic acid aptamer that specifically binds to a target molecule, such as a target protein.

In some embodiments, the target-binding moiety is a glycan. In some embodiments, the target-binding moiety includes a glycan epitope for an autoantibody.

### 4.5.1.1 Polypeptides

In some embodiments, e.g., of formula (XXI), the target-binding moiety is a polypeptide (e.g., peptide or protein target-binding motif, protein domain, engineered polypeptide, glycoprotein, antibody or antibody fragment) that specifically binds to a target molecule, such as a target protein. In some embodiments, the target-binding moiety of the bifunctional compound of this disclosure includes a polypeptide that binds to a soluble (e.g., secreted) target protein of interest. In some embodiments, the target-binding moiety is a polypeptide ligand for the target that includes a receptor ligand, or a receptor-binding portion or fragment of the receptor ligand, which binds a target cell surface receptor.

Depending on the source, target-binding polypeptides may contain L-amino acids, D-amino acids, or both and may contain any of a variety of naturally occurring amino acids, non-naturally occurring amino acids, and/or amino acid modifications or analogs known in the art. Useful modifications include, e.g., N-terminal acetylation, amidation, methylation, etc.

In certain embodiments, the polypeptide (P) of the conjugate comprises a polypeptide that binds to a soluble (e.g., secreted) target protein of interest. In certain embodiments, for example, the target protein of interest is a ligand that binds a cell surface receptor and P comprises the ligand binding portion of the cell surface receptor, or a bioisostere thereof, for example, the extracellular domain of the cell surface receptor, e.g., a ligand-binding domain of the extracellular domain of the cell surface receptor. In certain embodiments, target protein of interest is a cell surface receptor and P comprises a ligand that binds the cell surface receptor or a receptor-binding portion of the ligand, or a bioisostere thereof.

In some embodiments, the polypeptide (P) of the conjugate of this disclosure is a synthetic D-protein binder of a target protein of interest, e.g., a VEGF-A binding or PD1 binding D-protein as described in WO2020198074 and WO2020198075.

Conjugates of a polypeptide (i.e., Y is P), e.g., a conjugate of an antibody (Ab) and compound (Xn-L-Y, where Y is a chemoselective ligation group) may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In certain embodiments of the conjugates described herein, L is bonded through an amide bond to a lysine residue of P. In certain embodiments of the conjugates described herein, L is bonded through a thioether bond to a cysteine residue of P.

### 4.5.1.2 Antibodies

In some embodiments, e.g., of formula (XXI), the target-binding moiety is an antibody or antibody fragment that specifically binds to a target moiety, such as a target protein.

Accordingly, provided herein are conjugates of the following formula (XXII):

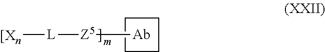

or a prodrug thereof, or a pharmaceutically acceptable salt thereof, wherein:
n is 1 to 20;
m is an average loading of 1 to 80;
each X is a moiety that binds to a cell surface M6PR (e.g., X is of formula (III) as described herein);
each L is a linker;
each Z⁵ is a residual moiety resulting from the covalent linkage of a chemoselective ligation group to a compatible group of Ab; and
Ab is the antibody or antibody fragment that specifically binds the target protein.

In some embodiments of formula (XXII), L is a linker (e.g., as described herein). In some embodiments of formula (XXII), Xn-L-Z⁵— is derived from a compound of formula (XIII) (e.g., as described herein), where Y is a chemoselective ligation group.

In some embodiments of formula (XXII), L is a linker of formula:

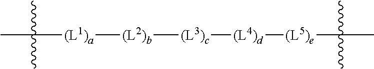

wherein L¹, L², L³, L⁴, L⁵, a, b, c, d, e, and n are defined herein.

In certain embodiments of formula (XXII), L is selected from the linkers of Tables 4-5.

In formula (XXII), Z⁵ can be any convenient residual moiety that results from the covalent linkage or conjugation of a chemoselective ligation group (Y) to a compatible reactive group of an antibody (Ab). In some instances, the compatible reactive group of antibody (Ab) is a group that can naturally be part on the biomolecule. In some instances, the compatible reactive group of antibody (Ab) is one that is introduced or incorporated into the biomolecule prior to conjugation. In such cases, the antibody (Ab) can be a modified version of a biomolecule. For example, a functional group (e.g., an amino group, a carboxylic acid group or a thiol group) of a biomolecule can be modified (e.g., using a chemical reagent such as 2-haloacetyl reagent, or 2-iminothiolane, or the like, or via coupling of a linker group including a chemoselective ligation group, such as an azide, alkyne, or the like) to introduce a compatible chemoselective ligation group.

In some embodiments of formula (XXII), Z⁵ is selected from

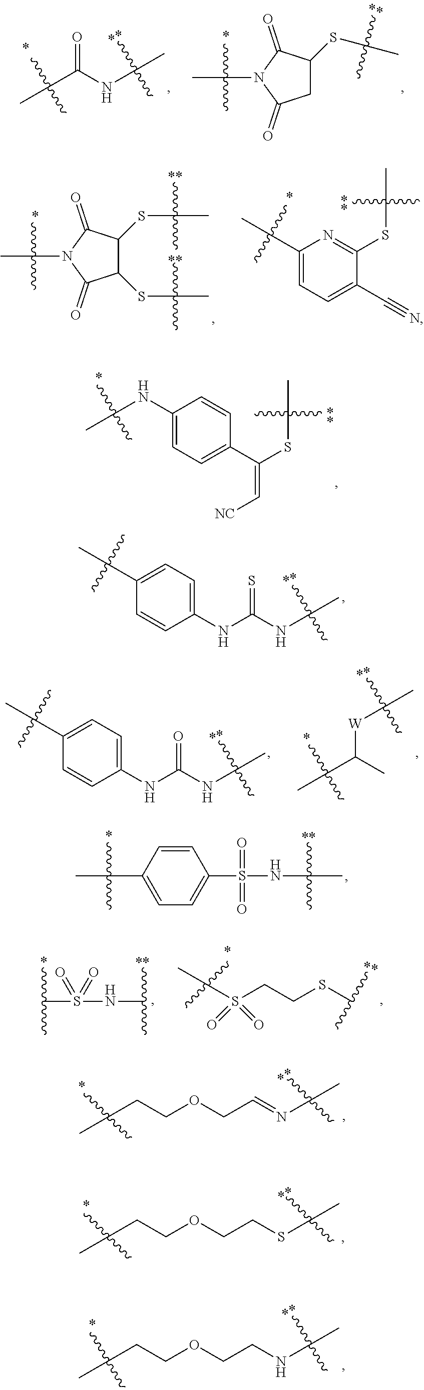

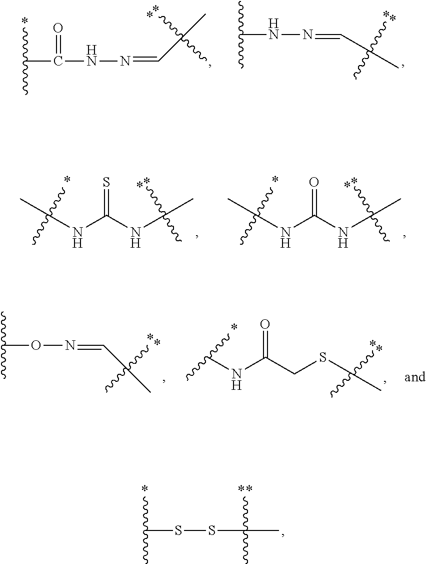

wherein [symbol] represents the point of attachment to the linker L,
wherein [symbol] represents the point of attachment to Ab,
W is CH₂, N, O or S; and
Ab is an antibody.

In certain embodiments of formula (XXII), Z⁵ is selected from

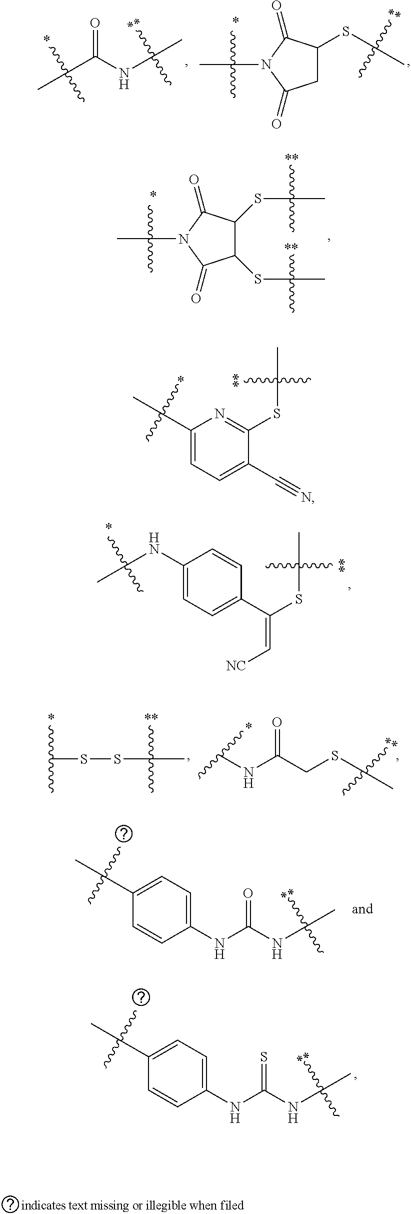

wherein [symbol] represents the point of attachment to L,
wherein [symbol] represents the point of attachment to Ab; and
Ab is an antibody.

In certain embodiments of formula (XXII), Z⁵ is selected from

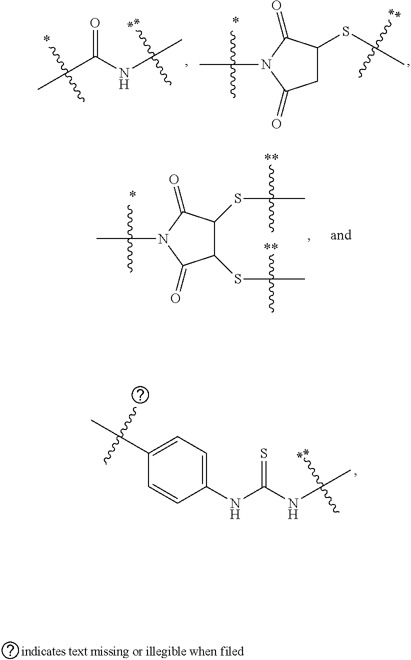

wherein [symbol] represents the point of attachment to L, wherein [symbol] represents the point of attachment to Ab.

In certain embodiments of formula (XXI)-(XXII), Z⁵ is derived from a chemoselective ligation group disclosed herein.

In certain embodiments of formula (XXI)-(XXII), n is 1. In certain embodiments, n is 2. In certain embodiments, n is 3. In certain embodiments, n is 4. In certain embodiments, n is 5.

The M6PR binding moiety can be site-specifically covalently linked to the antibody or antibody fragment, via an optional linking moiety. The M6PR binding moiety can be covalently linked to the antibody or antibody fragment via a site-specific cysteine modification on the antibody or antibody fragment (e.g., L443C) and a thiol-reactive chemoselective ligation group. The M6PR binding moiety can be covalently linked to the antibody or antibody fragment via one or more lysine residues of the antibody or antibody fragment and an amine-reactive chemoselective ligation group.

The M6PR binding moiety can be linked to the target-binding antibody or antibody fragment via a chimeric protein fusion, via an optional spacer sequence.

In some embodiments, the conjugate of this disclosure includes an antibody (Ab). In some embodiments, Ab is a monoclonal antibody. In some embodiments, Ab is a human antibody. In some embodiments, Ab is a humanized antibody. In some embodiments, Ab is a chimeric antibody. In some embodiments, Ab is a full-length antibody that includes two heavy chains and two light chains. In some embodiments, Ab is an IgG antibody, e.g., is an IgG1, IgG2, IgG3 or IgG4 antibody. In some embodiments, Ab is a single chain antibody. In some embodiments, the target-binding moiety is an antigen-binding fragment of an antibody, e.g., a Fab fragment.

In some embodiments, the antibody or antibody fragment specifically binds to a cancer antigen.

In some embodiments, the antibody or antibody fragment specifically binds to a hepatocyte antigen.

In some embodiments, the antibody or antibody fragment specifically binds to an antigen presented on a macrophage.

In some embodiments, the antibody or antibody fragment specifically binds to an intact complement or a fragment thereof. In some embodiments, the antibody or antibody fragment specifically binds to one or more immunodominant epitope(s) within intact complement or a fragment thereof.

In some embodiments, the antibody or antibody fragment specifically binds to a cell surface receptor. In some embodiments, the antibody or antibody fragment specifically binds to a cell surface receptor ligand.

In some embodiments, the antibody or antibody fragment specifically binds to an epidermal growth factor (EGF) protein, e.g., a human EGF. In some embodiments, the antibody or antibody fragment specifically binds to one or more immunodominant epitope(s) within an EGF protein.

In some embodiments, the antibody or antibody fragment specifically binds to an epidermal growth factor receptor (EGFR) protein, e.g., a human EGFR. In some embodiments, the antibody or antibody fragment specifically binds to one or more immunodominant epitope(s) within an EGFR protein. In some embodiments, the antibody or antibody fragment comprises the CDRs present in cetuximab. In some embodiments, the antibody or antibody fragment includes the variable light chain and variable heavy chain present in cetuximab. In some embodiments, the antibody is cetuximab. In some embodiments, the antibody or antibody fragment includes the CDRs present in matuzumab. In some embodiments, the antibody or antibody fragment includes the variable light chain and variable heavy chain present in matuzumab. In some embodiments, the antibody is matuzumab.

In some embodiments, the antibody or antibody fragment specifically binds to vascular endothelial growth factor (VEGF) protein, e.g., human VEGF protein. In some embodiments, the antibody or antibody fragment specifically binds to one or more immunodominant epitope(s) within a VEGF protein.

In some embodiments, the antibody or antibody fragment specifically binds to a vascular endothelial growth factor receptor (VEGFR) protein, e.g., human VEGFR protein. In some embodiments, the antibody or antibody fragment specifically binds vascular endothelial growth factor receptor 2 (VEGFR2) protein, e.g., a human VEGFR2 protein. In some embodiments, the antibody or antibody fragment specifically binds a vascular endothelial growth factor receptor 3 (VEGFR3) protein, e.g., a human VEGFR3 protein. In some embodiments, the antibody or antibody fragment specifically binds to one or more immunodominant epitope(s) within a VEGFR protein, a VEGFR2 protein or a VEGFR3 protein.

In some embodiments, the antibody or antibody fragment specifically binds to a fibroblast growth factor (FGF), e.g., a human FGF. In some embodiments, the antibody or antibody fragment specifically binds to one or more immunodominant epitope(s) within a FGF protein.

In some embodiments, the antibody or antibody fragment specifically binds to a fibroblast growth factor receptor (FGFR), e.g., a human FGFR. In some embodiments, the antibody or antibody fragment specifically binds fibroblast growth factor receptor 2 (FGFR2) protein, e.g., a human FGFR2 protein, for example, a FGFR2b protein. In some embodiments, the antibody or antibody fragment specifically binds a fibroblast growth factor receptor 3 (FGFR3) protein, e.g., a human FGFR3 protein. In some embodiments, the antibody or antibody fragment specifically binds to one or more immunodominant epitope(s) within a FGFR protein, a FGFR2 protein or a FGFR3 protein.

In some embodiments, the antibody specifically binds to a receptor tyrosine kinase cMET protein. In some embodiments, the antibody specifically binds to one or more immunodominant epitope(s) within a receptor tyrosine kinase cMET protein.

In some embodiments, the antibody specifically binds to a CD47 protein, e.g., a human CD47 protein. In some embodiments, the antibody specifically binds to one or more immunodominant epitope(s) within a CD47 protein.

In some embodiments, the antibody specifically binds to an immune checkpoint inhibitor. In some embodiments, the antibody binds to one or more immunodominant epitope(s) within an immune checkpoint inhibitor. In some embodiments, the antibody specifically binds to a programmed death protein, e.g., a human PD-1. In some embodiments, the antibody specifically binds to one or more immunodominant epitope(s) within PD-1 protein.

In some embodiments, the antibody specifically binds to a programmed death ligand-1 (PD-L1) protein, e.g., a human PD-L1. In some embodiments, the antibody specifically binds to one or more immunodominant epitope(s) within PD-L1 protein.

In some embodiments, the antibody binds to TIM3. In some embodiments, the antibody binds to one or more immunodominant epitope(s) within TIM3.

In some embodiments, the antibody specifically binds to a lectin. In some embodiments, the antibody specifically binds to one or more immunodominant epitope(s) within a lectin. In some embodiments, the antibody binds to SIGLEC. In some embodiments, the antibody binds to one or more immunodominant epitope(s) within SIGLEC. In some embodiments, the antibody binds to a cytokine receptor. In some embodiments, the antibody binds to a one or more immunodominant epitope(s) within cytokine receptor. In some embodiments, the antibody binds to sIL6R. In some embodiments, the antibody binds to one or more immunodominant epitope(s) within sIL6R. In some embodiments, the antibody binds to a cytokine. In some embodiments, the antibody binds to one or more immunodominant epitope(s) within a cytokine. In some embodiments, the antibody binds to MCP-1, TNF (e.g., a TNF-alpha), IL1a, IL1b, IL4, IL5, IL6, IL12/IL23, IL13, IL17 or p40. In some embodiments, the antibody binds to one or more immunodominant epitope(s) within MCP-1, TNF (e.g., a TNF-alpha), IL1a, IL1b, IL4, IL5, IL6, IL12/IL23, IL13, IL17 or p40.

In some embodiments, the antibody binds to a major histocompatibility protein (e.g., a MHC class I or class II molecule). In some embodiments, the antibody binds to one or more immunodominant epitope(s) within a major histocompatibility protein (e.g., a MHC class I or class II molecule). In some embodiments, the antibody binds to beta 2 microglobulin. In some embodiments, the antibody binds to one or more immunodominant epitope(s) within beta 2 microglobulin.

In some embodiments, the target-binding moiety is a biologic agent that is an antagonist of TNF protein (e.g., TNF-alpha). A number of biologic agents (e.g., monoclonal antibody drugs) have been developed to inhibit TNF binding to TNF receptors and shown to be clinically effective in a number of autoinflammatory diseases.

In certain embodiments of the conjugates described herein, L is bonded through an amide bond to a lysine residue of P. In certain embodiments of the conjugates described herein, L is bonded through a thioether bond to a cysteine residue of P. In certain embodiments of the conjugates described herein, L is bonded through an amide bond to a lysine residue of Ab, as depicted above. In certain embodiments of the conjugates described herein, L is bonded through a thioether bond to a cysteine residue of Ab, as depicted above. In certain embodiments of the conjugates described herein, L is bonded through two thioether bonds to two cysteine residues of Ab, wherein the two cysteine residues are from an opened cysteine-cysteine disulfide bond in Ab, as depicted above. In certain embodiments, the opened cysteine-cysteine disulfide bond is an interchain disulfide bond.

In certain embodiments of the conjugates described herein, when L is bonded through an amide bond to a lysine residue of P, m is an integer from 1 to 80. In certain embodiments of the conjugates described herein, when L is bonded through a thioether bond to a cysteine residue of P, m is an integer from 1 to 8.

In certain embodiments, conjugation to the polypeptide P or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety All such methodologies are contemplated for use in connection with making the conjugates described herein.

Loading of the compounds of formulas (I) and (III)-(IIIb) to the polypeptides (e.g., antibodies) described herein is represented by “m” in various formulas, and is the average number of units of “Xn-L-” or “Xn-” per conjugate molecule. As used herein, the term “DAR” refers to the average value of “m” or the loading of the conjugate. The number of “X” moieties (e.g., M6P moieties) per each unit of “Xn-L-” or “Xn-” is represented by “n” in formulas. The term “valency” or “valencies” refers to the number of “X” moieties per unit (“n”). It will be understood that loading, or DAR, is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for the conjugates described herein, the total number of “X” moieties per conjugate molecule will be n x m. As used herein, the term “total valency” or “total valencies” refers to the total number of “X” moieties per conjugate molecule (n x m; total valency).

DAR (loading) may range from 1 to 80 units per conjugate. The conjugates provided herein may include collections of polypeptides, antibodies or antigen binding fragments conjugated with a range of units, e.g., from 1 to 80. The average number of units per polypeptide or antibody in preparations of the conjugate from conjugation reactions may be characterized by conventional means such as mass spectroscopy. The quantitative distribution of DAR (loading) in terms of m may also be determined. In some instances, separation, purification, and characterization of homogeneous conjugate where m is a certain value may be achieved by means such as electrophoresis.

In certain embodiments, the DAR for a conjugate provided herein ranges from 1 to 80. In certain embodiments, the DAR for a conjugate provided herein ranges from 1 to 70. In certain embodiments, the DAR for a conjugate provided herein ranges from 1 to 60. In certain embodiments, the DAR for a conjugate provided herein ranges from 1 to 50. In certain embodiments, the DAR for a conjugate provided herein ranges from 1 to 40. In certain embodiments, the DAR for a conjugate provided herein ranges from 1 to 35. In certain embodiments, the DAR for a conjugate provided herein ranges from 1 to 30. In certain embodiments, the DAR for a conjugate provided herein ranges from 1 to 25. In certain embodiments, the DAR for a conjugate provided herein ranges from 1 to 20. In certain embodiments, the DAR for a conjugate provided herein ranges from 1 to 18. In certain embodiments, the DAR for a conjugate provided herein ranges from 1 to 15. In certain embodiments, the DAR for a conjugate provided herein ranges from 1 to 12. In certain embodiments, the DAR for a conjugate provided herein ranges from 1 to 10. In certain embodiments, the DAR for a conjugate provided herein ranges from 1 to 9. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4. In certain embodiments, the DAR for a conjugate provided herein ranges from 1 to 3. In certain embodiments, the DAR for a conjugate provided herein ranges from 2 to 12. In certain embodiments, the DAR for a conjugate provided herein ranges from 2 to 10. In certain embodiments, the DAR for a conjugate provided herein ranges from 2 to 9. In certain embodiments, the DAR for a conjugate provided herein ranges from 2 to 8. In certain embodiments, the DAR for a conjugate provided herein ranges from 2 to 7. In certain embodiments, the DAR for a conjugate provided herein ranges from 2 to 6. In certain embodiments, the DAR for a conjugate provided herein ranges from 2 to 5. In certain embodiments, the DAR for a conjugate provided herein ranges from 2 to 4. In certain embodiments, the DAR for a conjugate provided herein ranges from 3 to 12. In certain embodiments, the DAR for a conjugate provided herein ranges from 3 to 10. In certain embodiments, the DAR for a conjugate provided herein ranges from 3 to 9. In certain embodiments, the DAR for a conjugate provided herein ranges from 3 to 8. In certain embodiments, the DAR for a conjugate provided herein ranges from 3 to 7. In certain embodiments, the DAR for a conjugate provided herein ranges from 3 to 6. In certain embodiments, the DAR for a conjugate provided herein ranges from 3 to 5. In certain embodiments, the DAR for a conjugate provided herein ranges from 3 to 4.

In certain embodiments, the DAR for a conjugate provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the DAR for a conjugate provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9.

In some embodiments, the DAR for a conjugate provided herein ranges from 2 to 20, 2 to 19, 2 to 18, 2 to 17, 2 to 16, 2 to 15, 2 to 14, or 2 to 13. In some embodiments, the DAR for a conjugate provided herein ranges from 3 to 20, 3 to 19, 3 to 18, 3 to 17, 3 to 16, 3 to 15, 3 to 14, or 3 to 13. In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8. In some embodiments, the DAR for a conjugate provided herein is about 9. In some embodiments, the DAR for a conjugate provided herein is about 10. In some embodiments, the DAR for a conjugate provided herein is about 11. In some embodiments, the DAR for a conjugate provided herein is about 12. In some embodiments, the DAR for a conjugate provided herein is about 13. In some embodiments, the DAR for a conjugate provided herein is about 14. In some embodiments, the DAR for a conjugate provided herein is about 15. In some embodiments, the DAR for a conjugate provided herein is about 16. In some embodiments, the DAR for a conjugate provided herein is about 17. In some embodiments, the DAR for a conjugate provided herein is about 18. In some embodiments, the DAR for a conjugate provided herein is about 19. In some embodiments, the DAR for a conjugate provided herein is about 20.

In some embodiments, the DAR for a conjugate provided herein is about 25. In some embodiments, the DAR for a conjugate provided herein is about 30. In some embodiments, the DAR for a conjugate provided herein is about 35. In some embodiments, the DAR for a conjugate provided herein is about 40. In some embodiments, the DAR for a conjugate provided herein is about 50. In some embodiments, the DAR for a conjugate provided herein is about 60. In some embodiments, the DAR for a conjugate provided herein is about 70. In some embodiments, the DAR for a conjugate provided herein is about 80.

In certain embodiments, fewer than the theoretical maximum of units are conjugated to the polypeptide, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### 4.5.1.3 Small Molecules

In some embodiments, the target-binding moiety of a bifunctional compound of this disclosure is a small molecule that specifically binds to a target molecule, such as a target protein. In some embodiments, the bifunctional compound includes a small molecule inhibitor or ligand of a target protein. A small molecule target-binding moiety can be covalently linked to one or more M6PR binding moieties via a linker. The linker can be attached to the small molecule via substitution at any suitable site of the small molecule such that binding to the target protein is substantially retained.

In some embodiments, the target-binding moiety is a small molecule inhibitor or antagonist of a target protein (e.g., as described herein). Any convenient small molecules known to bind a target of interest can be adapted for use in the subject compounds and conjugates.

In some embodiments, the target-binding moiety is a small molecule inhibitor or antagonist of VEGF. In some embodiments, the target-binding moiety is a small molecule inhibitor or antagonist of PD-L1.

In some embodiments, the target-binding moiety is a small molecule inhibitor or antagonist of EGFR protein, a VEGFR protein, a FGFR2 protein or a FGFR3 protein.

In some embodiments, the target-binding moiety is a small molecule inhibitor or antagonist of TNF protein (e.g., TNF-alpha). TNF-alpha (TNFα) is a soluble cytokine produced by monocytes and macrophages as part of immune and inflammatory processes and is involved in a diverse range of cellular responses including differentiation, proliferation, inflammation, and cell death. TNFα is a type II transmembrane protein that can be cleaved and secreted as a soluble form. Both the transmembrane and soluble biologically active forms of TNFα are homotrimeric complexes that can signal through TNF receptors 1 and 2 (TNF-R¹ and TNF-R²). TNFα is directly involved in systemic inflammation through the regulation of the intracellular NF-κB, JNK and p38-MAPK signaling pathways.

The TNFα binding moiety can be a TNFα inhibitor, such as a competitive inhibitor of TNF receptor binding or an allosteric inhibitor of TNF signaling. The compounds of this disclosure can include a potent TNFα inhibitor, e.g., an inhibitor having sub-micromolar inhibitory activity. In some embodiments, the TNFα inhibitor is an allosteric inhibitor. In some embodiments, the TNFα binding moiety is an allosteric desymmetrization TNFα inhibitor. An allosteric desymmetrization TNFα inhibitor refers to a compound that binds to an allosteric site within TNFα and stabilizes the trimeric unit in a nonsymmetrical conformation that allows the TNFα trimer to recruit only two out of the three copies of TNF Receptor (TNFR, e.g., TNFR1), leading to an incompetent TNFα-TNFR signaling complex.

See e.g., Xiao et al. in Journal of Medicinal Chemistry 2020 63 (23), 15050-15071, and McMillan et al. in Nature Communications (2021) 12:582, which discloses an analysis of the X-ray co-crystal structure of exemplary inhibitors bound to TNFα. An allosteric desymmetrization TNFα inhibitor can act via a particular mechanism of action to provide potent inhibitory activity. For example, (a) the TNFα inhibitor binding site is a cavity within the TNFα trimer created via movement of monomer A, (b) the inhibitor stabilizes the TNFα trimer in an inactive conformation by forming key 7r-K and hydrogen bonding interactions, (c) an allosteric desymmetrization TNFα inhibitor binds to TNFα trimer leading to major disruption of one TNFR binding site and minor disruption of a second site, while the third site remains unchanged, and (d) the allosteric desymmetrization TNFα inhibitor modulates TNF-R activity through an allosteric mechanism rather than direct competition with TNFR. Thus, the binding of an allosteric desymmetrization TNFα inhibitor to the symmetric TNFα trimer can lead to the formation of an asymmetric trimer which prevents the recruitment of three TNF receptor molecules that are necessary for signaling.

### 4.5.2. Targets

As summarized above, the bifunctional compounds of this disclosure can include a moiety of interest (Y) that specifically binds a target molecule. The target molecule can be a cell surface molecule or an extracellular molecule.

In some embodiments of the compounds and methods of this disclosure, the target molecule is a cell surface molecule. By “cell surface molecule” is meant a target molecule associated with a cell membrane, e.g., because the molecule has a domain that inserts into or spans a cell membrane, e.g., a cell membrane-tethering domain or a transmembrane domain. The cell surface molecule may be any cell surface molecule which is desired for targeted degradation via the endosomal/lysosomal pathway. In some embodiments, the cell surface molecule is a cell surface receptor.

Cell surface receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1). In some embodiments, the cell surface molecule is CD71 (transferrin receptor). In certain aspects, the cell surface receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In some embodiments, the moiety of interest (Y) specifically binds a cell surface molecule which mediates its effect not through a specific molecular interaction (and therefore is not susceptible to blocking), but rather through bulk biophysical or aggregate effects. A non-limiting example of such a cell surface molecule is a mucin. Examples of mucins include, but are not limited to, MUC1, MUC16, MUC2, MUC5AC, MUC4, CD43, CD45, GPIb, and the like.

In some embodiments, when the moiety of interest specifically binds a cell surface molecule, the cell surface molecule is present on a cancer cell. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the cell surface molecule present on the cancer cell is a tumor-associated antigen or a tumor-specific antigen. In certain aspects, when the moiety of interest (Y) specifically binds a cell surface molecule, the cell surface molecule is present on an immune cell. In some embodiments, the cell surface molecule is present on an immune cell selected from a T cell, a B cell, a natural killer (NK) cell, a macrophage, a monocyte, a neutrophil, a dendritic cell, a mast cell, a basophil, and an eosinophil. In certain aspects, the cell surface molecule present on the immune cell is an inhibitory immune receptor. As used herein, an “inhibitory immune receptor” is a receptor present on an immune cell that negatively regulates an immune response. Examples of inhibitory immune receptors which may be inhibited according to the methods of the present disclosure include inhibitory immune receptors of the Ig superfamily, including but not limited to: CD200R, CD300a (IRp60; mouse MAIR-I), CD300f (IREM-1), CEACAM1 (CD66a), FcγRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-a (FDF03), SIRL-1, and SIRP-a. Further examples of inhibitory immune receptors which may be inhibited according to the methods of the present disclosure include sialic acid-binding Ig-like lectin (Siglec) receptors, e.g., Siglec 7, Siglec 9, and/or the like. Additional examples of inhibitory immune receptors which may be inhibited according to the methods of the present disclosure include C-type lectins, including but not limited to: CLEC4A (DCIR), Ly49Q and MICL. Details regarding inhibitory immune receptors may be found, e.g., in Steevels et al. (2011) Eur. J. Immunol. 41 (3):575-587. In some embodiments, the cell surface molecule present on the immune cell is a ligand of an inhibitory immune receptor. In certain aspects, the cell surface molecule present on the immune cell is an immune checkpoint molecule. Non-limiting examples of immune checkpoint molecules to which the moiety of interest (Y) may specifically bind include PD-1, PD-L1, CTLA4, TIM3, LAG3, TIGIT, and a member of the B7 family.

In some embodiments of the compounds and methods of this disclosure, the target molecule is an extracellular molecule. By “extracellular molecule” is meant a soluble molecule external to the cell membranes of any cells in the vicinity of the soluble molecule. The extracellular molecule may be any extracellular molecule which is desired for targeted degradation via the endosomal/lysosomal pathway.

In some embodiments, the extracellular molecule is a soluble target protein. In some embodiments, the extracellular molecule is a secreted protein that accumulates in disease (e.g., alpha-synuclein), a cholesterol carrier (e.g., ApoB), an infectious disease toxin (e.g., AB toxins, ESAT-6), an infectious particle (e.g., a whole virus, a whole bacterium, etc.), a clotting factor (e.g., Factor IX), the target of any FDA approved antibody that binds to an extracellular molecule (e.g., TNFalpha), any chemokine or cytokine (e.g., mediators of sepsis or chronic inflammation such at IL-1), a proteinaceous hormone (e.g., insulin, ACTH, etc.), a proteinaceous mediator of a mood disorder, a proteinaceous mediator of energy homeostasis (e.g., leptin, ghrelin, etc.), a proteinaceous allergen present in the bloodstream or an antibody against such an allergen (e.g., for peanut allergies), a proteinaceous toxin (e.g., snake venom hyaluronidase, etc.), an autoantibody, etc.

In some embodiments, the target molecule is an extracellular molecule that is an antibody, e.g., an antibody that specifically binds a cell surface molecule or different extracellular molecule. In some embodiments, the antibody is an autoantibody. In some embodiments, the target is a human immunoglobulin A(IgA). In some embodiments, the IgA is a particular antibody that plays a crucial role in the immune function of mucous membranes. In the blood, IgA interacts with an Fc receptor called CD89 expressed on immune effector cells, to initiate inflammatory reactions. Aberrant IgA expression has been implicated in a number of autoimmune and immune-mediated disorders. In some embodiments, the target is a human immunoglobulin G (IgG). The Fc regions of IgGs include a conserved N-glycosylation site at asparagine 297 in the constant region of the heavy chain. Various N-glycans can be attached to this site. The N-glycan IgG composition has been linked to several autoimmune, infectious and metabolic diseases. In addition, overexpression of IgG4 has been associated with IG4-related diseases. In some embodiments, the target is human immunoglobulin E (IgE). IgE is a type of immunoglobulin that plays an essential role in type I hypersensitivity, which can manifest into various allergic diseases and conditions.

In some embodiments, the extracellular molecule is a ligand for a cell surface receptor. Cell surface receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor b (TGF-b), including any particular subtypes of such cytokines), hormones, and the like. In certain aspects, the moiety of interest (Y) specifically binds apolipoprotein E4 (ApoE4).

### 4.5.3. Moiety of Interest for Intracellular Delivery

In some embodiments, the moiety of interest is a molecule that does not bind to an extracellular target, but rather is a molecule that is itself desirable to deliver intracellularly. In some embodiments, the moiety of interest is selected from enzymes (e.g., lysosomal enzyme), a nanoparticle, a viral composition (e.g., viral particle), therapeutic protein, therapeutic antibodies.

In some embodiments, the moiety of interest Y is selected from small molecule, small molecule drug, chemotherapeutic agent, cytotoxic agent, diagnostic agent, dye, fluorophore, and the like.

In some embodiments, the moiety of interest Y is a nanoparticle suitable for delivery of one or more agents or cargo within the nanoparticle.

### 4.5.3.1 Conjugates for Enzyme Replacement Therapy

In some embodiments, the moiety of interest is a lysosomal enzyme for delivery to a cell for use in enzyme replacement therapy, such as acid alpha-glucosidase (GAA). Lysosomal enzymes of interest that may be adapted for use in conjugates of this disclosure include, but are not limited to, acid alpha-glucosidase, acid beta-galactosidase-1, acid sphingomyelinase, alpha-D-mannosidase, alpha-fucosidase, alpha-galactosidase A, alpha-glucosaminide acetyltransferase, alpha-glucosidase, alpha-L-iduronidase, alpha-N-acetylgalactosaminidase, alpha-acetylglucosaminidase, alpha-D-neuraminidase, arylsulfatase A, arylsulfatase B, beta-galactosidase, beta-glucuronidase, beta-mannosidase, cathepsin D, cathepsin K, ceramidase, cystinosine, ganglioside activator GM2, galactocerebrosidase, glucocerebrosidase, heparan sulfatase, hexosaminidase A, hexosaminidase B, hyaluronidase, iduronate-2-sulfatase, LAMP2, lysosomal acid lipase, N-acetylglucosamine-1-phosphotransferase, N-acetylgalactosamine 6-sulfatase, N-acetylglucosamine-1-phosphotransferase, N-acetylglucosamine-6-sulfate sulfatase, N-aspartyl-beta-glucosaminidase, palmitoyl-thioesterase-1, acid phosphatase, protected protein/cathepsin A (PPCA), sialin, tripeptidyl-peptidase 1.

Conjugation to an enzyme can be achieved using the methods described here for preparing polypeptide and antibody conjugates.

### 4.5.3.2 Modified Viral Compositions for Viral Transduction

In specific embodiments, Y is a viral composition that includes a viral particle, viral capsid, a viral envelope or a viral protein. In some embodiments, the viral composition is a viral particle that comprises a transgene. In some embodiments, the viral protein is a viral capsid protein or a viral envelope protein. Conjugation of one or several compounds of this disclosure with a viral composition produces a modified viral composition that provides for enhanced viral transduction as compared to an unlabeled viral composition.

In certain aspects, provided herein are modified viral compositions comprising a viral composition, for example, a virus particle, a virus capsid or a viral protein (e.g., a viral capsid protein or an envelope protein) attached to (e.g., conjugated to, directly or indirectly, for example via an intervening linker sequence) a M6PR binding moiety that binds to a cell surface receptor. In certain embodiments, a modified viral composition comprises a virus particle that comprises a polynucleotide that optionally comprises a transgene, e.g., a transgene useful for therapeutic applications.

The modified viral compositions, e.g., viral conjugates, presented herein may comprise any viral composition described herein e.g., any virus particle, capsid or viral protein, for example capsid protein or envelope protein, or fragment thereof, as described herein.

In certain aspects, a viral composition described herein may comprise a virus particle. The terms “virus particle,” “viral particle,” “virus vector” or “viral vector” are used interchangeably herein. A “virus particle” refers to a virus capsid and a polynucleotide (DNA or RNA), which may comprise a viral genome, a portion of a viral genome, or a polynucleotide derived from a viral genome (e.g., one or more ITRs), which polynucleotide optionally comprises a transgene. In certain instances, a virus particle further comprises an envelope (which generally comprises lipid moieties and envelope proteins), surrounding or partially surrounding the capsid.

A viral particle may be referred to as a “recombinant viral particle,” or “recombinant virus particle,” which terms as used herein refer to a virus particle that has been genetically altered, e.g., by the deletion or other mutation of an endogenous viral gene and/or the addition or insertion of a heterologous nucleic acid construct into the polynucleotide of the virus particle. Thus, a recombinant virus particle generally refers to a virus particle comprising a capsid coat or shell (and an optional outer envelope) within which is packaged a polynucleotide sequence that comprises sequences of viral origin and sequences not of viral origin (i.e., a polynucleotide heterologous to the virus). This polynucleotide sequence is typically a sequence of interest for the genetic alteration of a cell.

In certain aspects, a viral composition described herein may comprise an “viral capsid,” “empty viral particle,” “empty virus particle,” or “capsid,” or “empty particle” when referred to herein in the context of the virus, which terms as used herein refer to a three-dimensional shell or coat comprising a viral capsid protein, optionally surrounded or partially surrounded by an outer envelope. In particular embodiments, the viral composition is a virus particle or a fragment thereof, virus capsid or fragment thereof, a viral protein, for example, a virus capsid protein or fragment thereof or envelope protein, or fragment thereof.

In some embodiments, the virus used in a modified viral composition provided herein is adenovirus (AV); adeno-associated virus (AAV); retroviruses (e.g., lentiviruses (LV), rhabdoviruses, murine leukemia virus); herpes simplex virus, coronavirus, reovirus, and the like. In some embodiments, the viral vector, viral particle or viral protein used in the present disclosure is derived from a non-enveloped virus, e.g., an adeno-associated virus (AAV).

In some embodiments, lentiviral vectors can be used for CAR-T gene delivery, vaccines, or research tools, e.g., to introduce genes into mature T cells to generate immunity to cancer through the delivery of chimeric antigen receptors (CARs) or cloned T-cell receptors.

Naturally occurring AAV forms a virus particle that comprises a three-dimensional capsid coat or shell (a “capsid”) made up of capsid proteins (VP1, VP2 and VP3) and, contained within the capsid, an AAV viral genome.

The modified AAV compositions, e.g., AAV conjugates or fusions, presented herein may comprise any AAV composition described herein, e.g., any AAV particle, capsid or capsid protein, or fragment thereof, as described herein. The term “AAV capsid protein” or “AAV cap protein” refers to a protein encoded by an AAV capsid (cap) gene (e.g., VP1, VP2, and VP3) or a variant or fragment thereof. The term includes a capsid protein expressed by or derived from an AAV, e.g., a recombinant AAV, such as a chimeric AAV. For example, the term includes but not limited to a capsid protein derived from any AAV serotype such as AAV1, AAV2, AAV2i8, AAV3, AAV4, AAV5, AAV6, AAV7, AAV8, AAV9, AAV10, AAV rh10, AAV11, AAV12, AAV13, AAV-DJ, AAV3b, AAV LK03, AAV rh74, AAV Anc81, Anc82, Anc83, Anc84, Anc110, Anc113, Anc126, or Anc127, AAV_go.1, AAV hu.37, or AAV rh.8 or a variant thereof.

### 4.5.3.3 Bridging Moieties that Bind Virus Compositions

In some embodiments, Y is a bridging moiety that specifically binds to a viral composition described above, for example, a viral particle, viral capsid, viral envelope or viral protein (e.g., a viral capsid protein or envelope protein), wherein the binding is not via a covalent linkage. Such conjugates can be useful in enhancing intracellular delivery and viral transduction of a target virus composition.

Any suitable moiety that binds a viral particle, viral capsid, viral envelope or viral protein (e.g., a viral capsid protein or envelope protein) can be adapted for use in the bridging moiety conjugates of this disclosure.

In certain embodiments, a bridging moiety is a polypeptide that specifically binds a viral composition. In some embodiments, the bridging moiety is a polypeptide that binds to a viral composition, e.g., a virus particle, virus capsid, virus envelope, or a viral protein, for example, a viral capsid protein or viral envelope protein. In certain aspects, the bridging composition binds the viral capsid protein or a viral envelope protein, when the viral protein is part of a virus particle.

In certain embodiments, a bridging moiety is an antibody or antibody fragment (e.g., an antigen binding fragment of an antibody) that specifically binds a viral composition. In certain embodiments, a bridging moiety that binds a viral protein may also bind a viral particle, for example, via binding to the viral protein incorporated in a viral particle. Likewise, in certain embodiments, a bridging moiety that binds a viral particle may also bind a viral protein even if the viral protein is not incorporated in a viral particle. The viral particle can be an AAV virus particle. The viral protein can be a AAV capsid protein.

In some embodiments, the bridging moieties of this disclosure specifically bind to an AAV composition, e.g., an AAV particle, AAV capsid, or AAV viral protein (e.g., an AAV capsid protein, for example, a VP1, VP2 or VP3 protein).

An antibody or antigen binding fragment that may be utilized in connection with the modified viral compositions provided herein, e.g., in connection with the bridging compositions and bridging moieties presented herein, includes, without limitation, monoclonal antibodies, antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies).

### 4.6. Exemplary Conjugates

Exemplary monomeric compounds of this disclosure that include a chemoselective ligation group are shown in Table 7 which can be used to prepare conjugates of moieties of interest.

TABLE 7
Exemplary ligand-linker compounds for use in preparing conjugates
Xₙ-L-Y

| | | | | effective length | Chemoselective |
|---|---|---|---|---|---|
| Cmpd # | X | L | n | L to Y conjugate | ligation |
| 513 (I-39) | X1 | 1.11 | 1 | 23 | PFP ester |
| 521 | X2 | 1.5 | 1 | 22 | NHS ester |
| 529 (I-38) | X3 | 1.5 | 1 | 22 | PFP ester |
| 558 | X3 | 1.5 | 1 | 22 | TFP ester |
| 546 | X3 | 5.1 | 1 | 40 | maleimide |
| 533 (I-61) | X4 | 1.8 | 1 | 25 | PFP ester |
| 560 | X32 | 1.5 | 1 | 22 | PFP ester |
| 562 | X36 | 1.5 | 1 | 22 | PFP ester |
| 563 | X35 | 1.5 | 1 | 22 | PFP ester |
| 564 | X36 | 1.6 | 1 | 20 | PFP ester |
| 565 | X37 | 1.5 | 1 | 22 | PFP ester |
| 566 | X28 | 1.5 | 1 | 22 | PFP ester |
| 568 | X27 | 1.5 | 1 | 22 | PFP ester |
| 548 (I-91) | X7 | 7.2 | 1 | 23 | PFP ester |
| 547 | X6 | 7.2 | 1 | 23 | NHS ester |
| 535 (I-88) | X8 | 1.5 | 1 | 22 | PFP ester |
| 551 (I-94) | X9 | 1.7 | 1 | 21 | PFP ester |
| 570 | X11* | 1.5 | 1 | 22 | PFP ester |
| | (beta S) | | | | |
| 552 (I-95) | X11 | 1.5 | 1 | 22 | PFP ester |
| | (alpha S) | | | | |
| 545 (I-87) | X12 | 1.5 | 1 | 22 | PFP ester |
| 544 (I-86) | X13 | 1.5 | 1 | 22 | PFP ester |
| 549 (I-92) | X15 | 1.5 | 1 | 22 | PFP ester |
| 550 (I-93) | X16 | 1.5 | 1 | 22 | PFP ester |
| 538 (I-64) | X18 | 1.5 | 1 | 22 | PFP ester |
| 536 (I-60) | X19 | 1.5 | 1 | 22 | PFP ester |
| 537 (I-66) | X22 | 1.5 | 1 | 22 | PFP ester |
| 556 (I-104) | X25 | 1.5 | 1 | 22 | PFP ester |
| 567 | X26 | 7.1 | 1 | 15 | PFP ester |
| 559 | X22 | 1.5 | 1 | 22 | PFP ester |

PFP is pentafluorophenyl
TFP is tetrafluorophenyl
NHS is N-hydroxysuccinimde ester

Exemplary dimeric (n=2) compounds of this disclosure are shown in Table 8 that include a chemoselective ligation group and can be used to prepare conjugates of moieties of interest.

TABLE 8
Exemplary Multimeric Ligand-linker compounds
Xₙ-L-Y

| Cmpd # | X | n | L1 to branch length | branch to Y length | Y |
|---|---|---|---|---|---|
| 711 | X3 | 2 | 18 to C | 17 to C═O | maleimide |
| 711 | X3 | 2 | 18 to C | 17 to C═O | PFP ester |
| 710 | X16 | 2 | 18 to N | 18 to C═C | Maleimide |
| 713 | X3 | 2 | 17 or 18 to CH | 93 to C═O | PFP ester |
| 716 (I-12) | X104 | 2 | 16 to N | 11 to C═O | PFP ester |

The structures of exemplary monomeric (n=1) compounds of this disclosure are shown in Table 9 that include a chemoselective ligation group and which can be used to prepare conjugates of moieties of interest.

TABLE 9
Exemplary M6PR binding ligand-linker compounds for conjugates

| Table # | |
|---|---|
| (Ex. #) | Compound structure |
| 501 (I-1) | 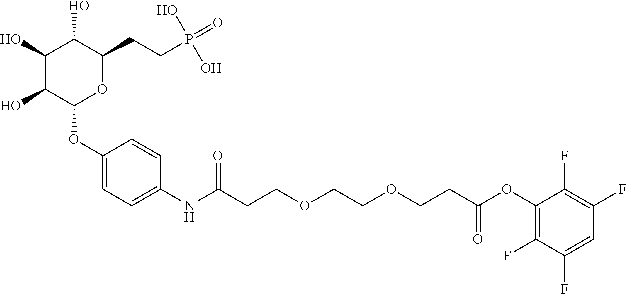 [SMILES: O=C(CCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 502 | 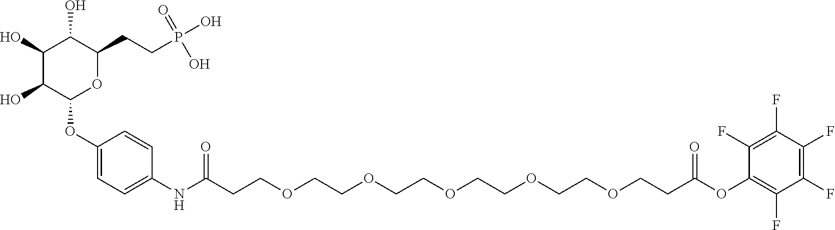 [SMILES: O=C(CCOCCOCCOCCOCCOCCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 503 | 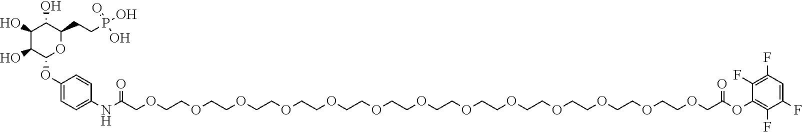 [SMILES: O=C(COCCOCCOCCOCCOCCOCCOCCOCCOCCOCCOCCOCCOCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 504 | 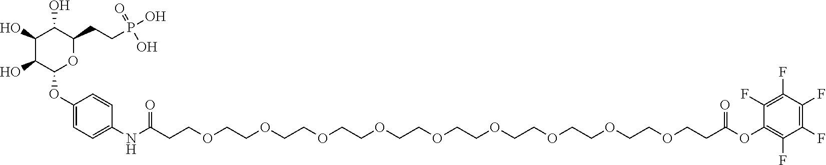 [SMILES: O=C(CCOCCOCCOCCOCCOCCOCCOCCOCCOCCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 505 (I-2) | 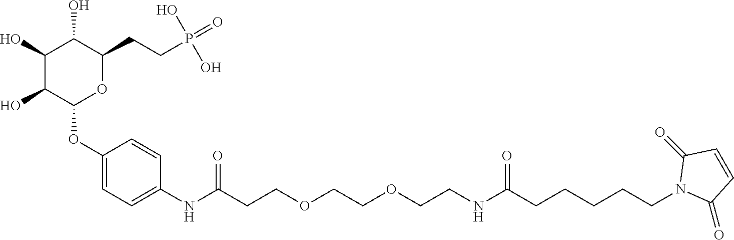 [SMILES: O=C(CCCCCN1C(=O)C=CC1=O)NCCOCCOCCC(=O)Nc1ccc(O[C@H]2O[C@H](CCP(=O)(O)O)[C@@H](O)[C@H](O)[C@@H]2O)cc1] |
| 506 | 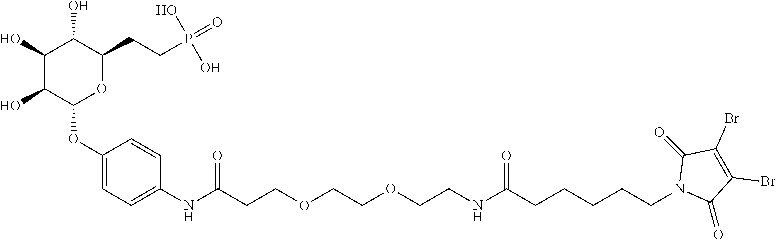 [SMILES: O=C(CCCCCN1C(=O)C(Br)=C(Br)C1=O)NCCOCCOCCC(=O)Nc1ccc(O[C@H]2O[C@H](CCP(=O)(O)O)[C@@H](O)[C@H](O)[C@@H]2O)cc1] |
| 507 | 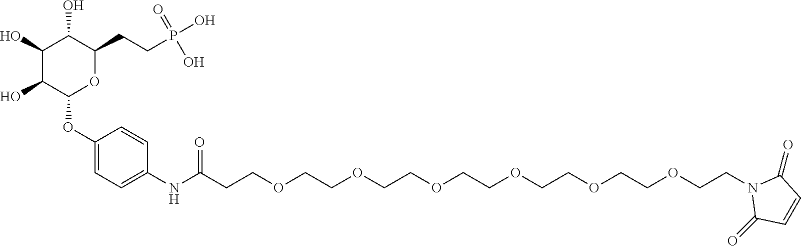 [SMILES: O=C(CCOCCOCCOCCOCCOCCOCCN1C(=O)C=CC1=O)Nc1ccc(O[C@H]2O[C@H](CCP(=O)(O)O)[C@@H](O)[C@H](O)[C@@H]2O)cc1] |
| 508 (I-3) | 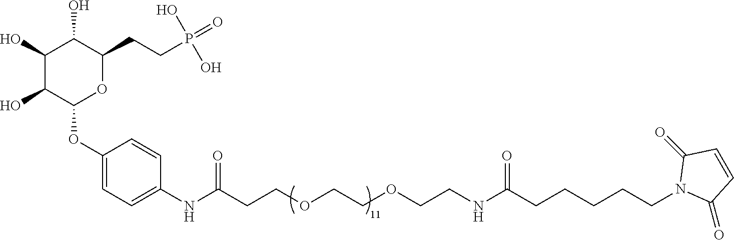 |
| 509 | 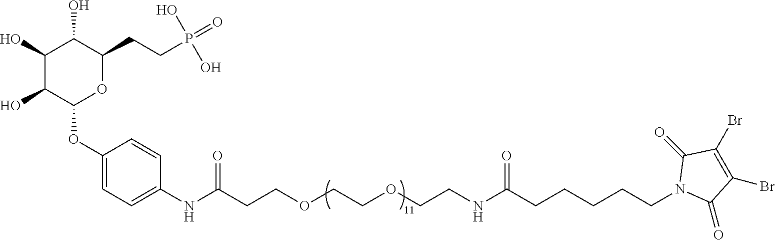 |
| 510 | 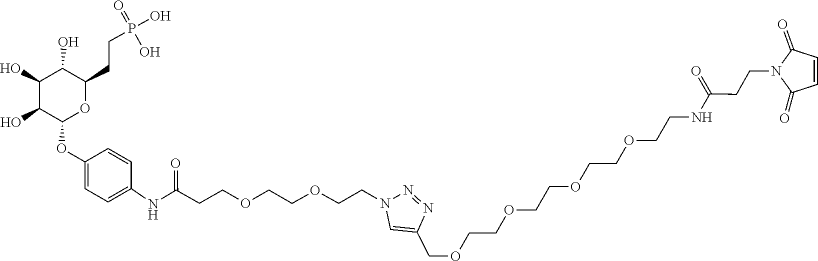 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 511 (I-4) | 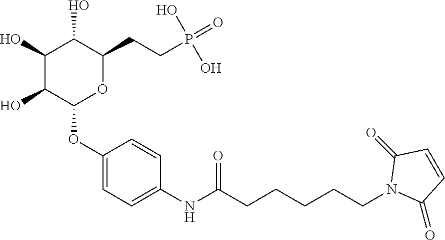 [SMILES: O=C(CCCCCN1C(=O)C=CC1=O)Nc1ccc(O[C@H]2O[C@H](CCP(=O)(O)O)[C@@H](O)[C@H](O)[C@@H]2O)cc1] |
| 512 (I-5) | 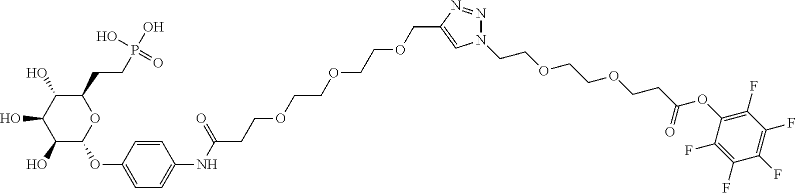 [SMILES: O=C(CCOCCOCCOCc1cn(CCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 513 (I-39) | 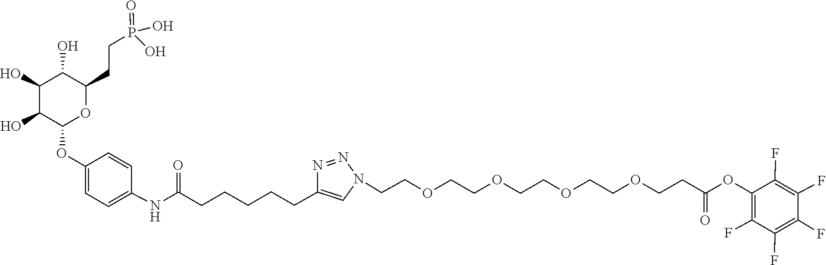 [SMILES: O=C(CCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 514 (I-57) | 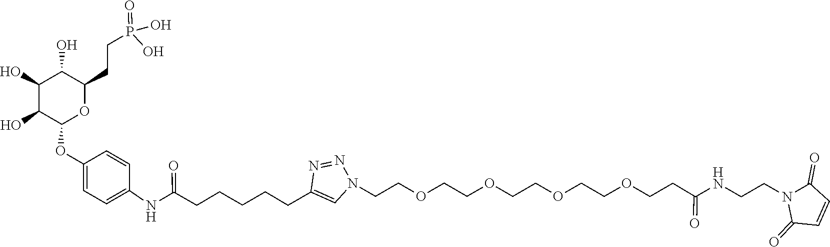 [SMILES: O=C(CCOCCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 515 (I-16) | 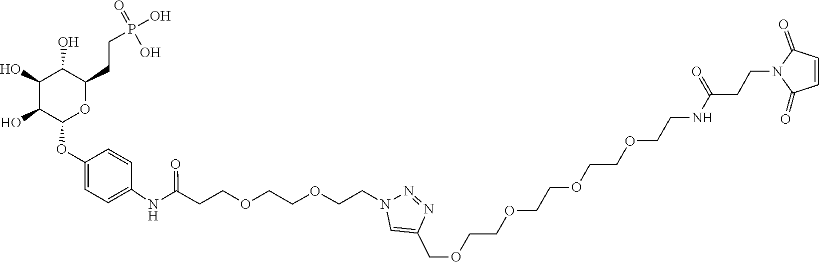 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 516 (I-6) | 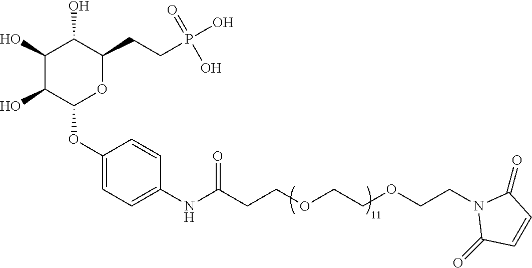 |
| 519 (I-47) | 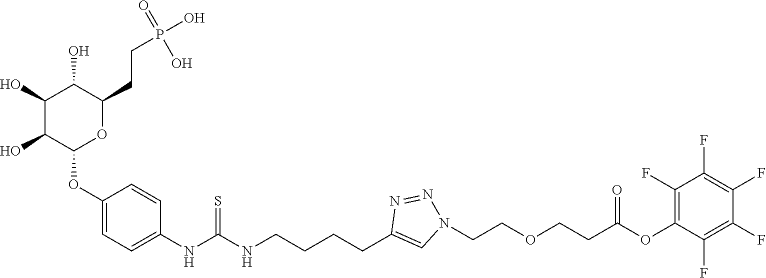 [SMILES: O=C(CCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 520 (I-7) | 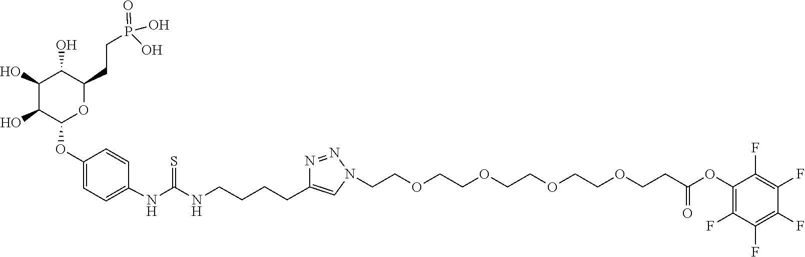 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 521 | 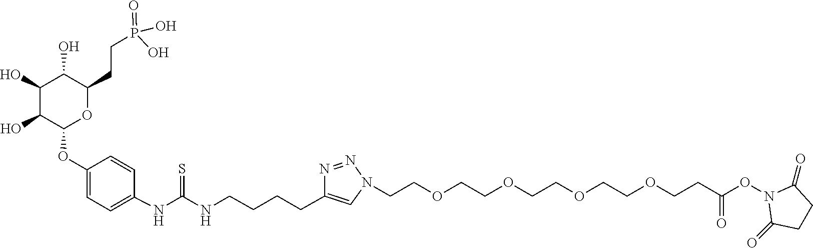 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)ON1C(=O)CCC1=O] |
| 522 (I-49) | 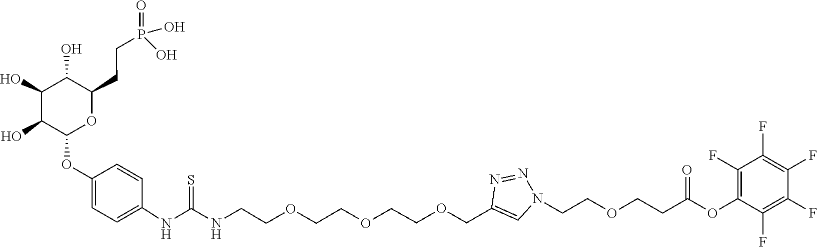 [SMILES: O=C(CCOCCn1cc(COCCOCCOCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 523 (I-17) | 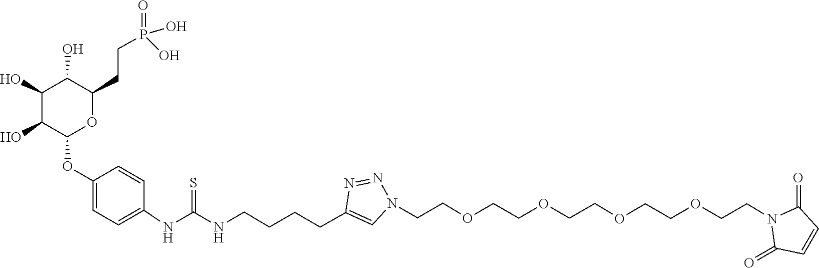 [SMILES: O=C1C=CC(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 524 (I-18) | 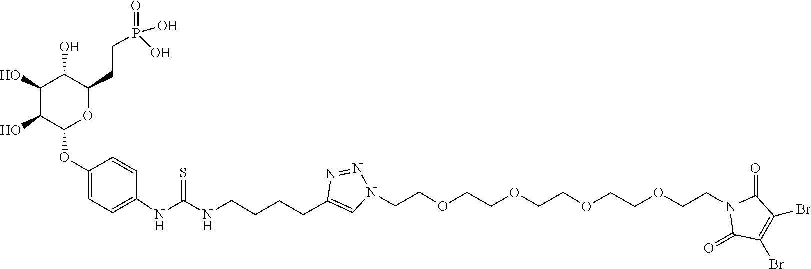 [SMILES: O=C1C(Br)=C(Br)C(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 525 | 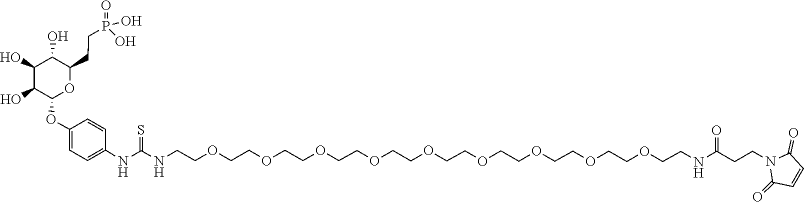 [SMILES: O=C(CCN1C(=O)C=CC1=O)NCCOCCOCCOCCOCCOCCOCCOCCOCCOCCNC(=S)Nc1ccc(O[C@H]2O[C@H](CCP(=O)(O)O)[C@@H](O)[C@H](O)[C@@H]2O)cc1] |
| 526 (I-48) | 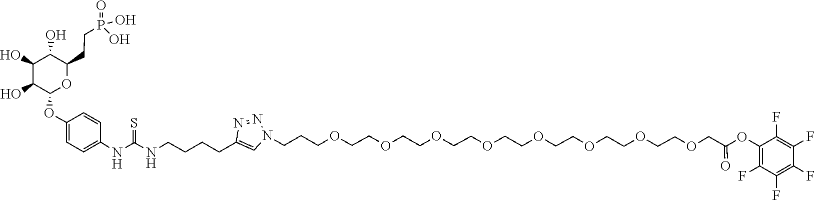 [SMILES: O=C(COCCOCCOCCOCCOCCOCCOCCOCCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 527 | 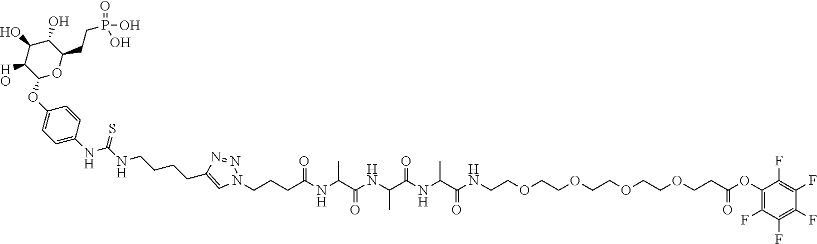 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)Oc1c(F)c(F)c(F)c(F)c1F] |
| 528 (I-51) | 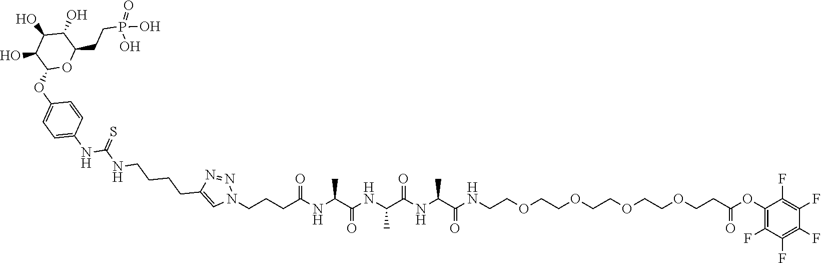 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)Oc1c(F)c(F)c(F)c(F)c1F] |
| 529 (I-38) | 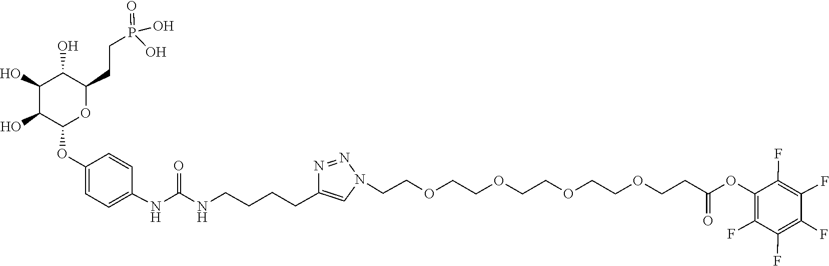 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 530 (I-50) | 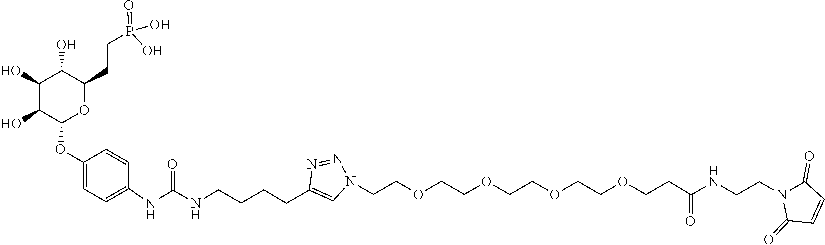 [SMILES: O=C(CCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 531 | 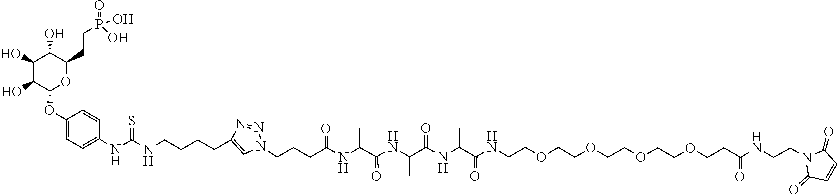 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)NCCN1C(=O)C=CC1=O] |
| 532 (I-55) | 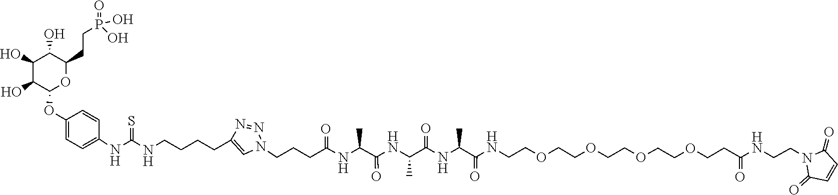 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)NCCN1C(=O)C=CC1=O] |
| 533 (I-61) | 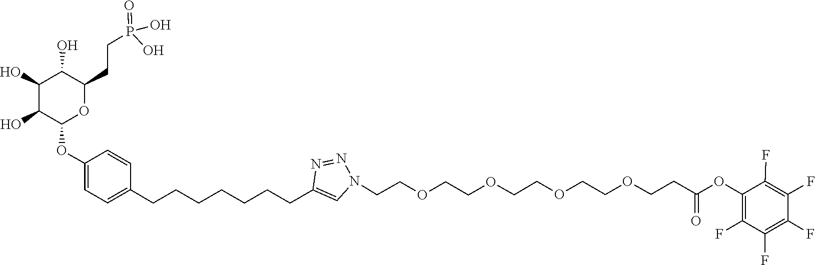 [SMILES: O=C(CCOCCOCCOCCOCCn1cc(CCCCCCCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 534 (I-62) | 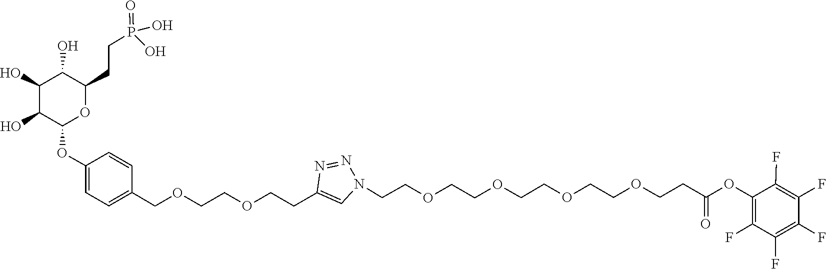 [SMILES: O=C(CCOCCOCCOCCOCCn1cc(CCOCCOCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 535 (I-63) (I-88) | 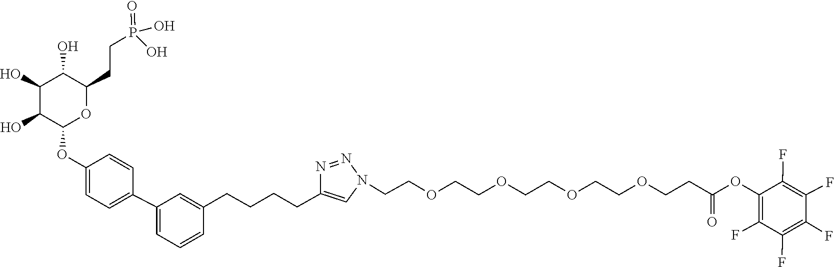 [SMILES: O=C(CCOCCOCCOCCOCCn1cc(CCCCc2cccc(-c3ccc(O[C@H]4O[C@H](CCP(=O)(O)O)[C@@H](O)[C@H](O)[C@@H]4O)cc3)c2)nn1)Oc1c(F)c(F)c(F)c(F)c1F] |
| 536 (I-60) | 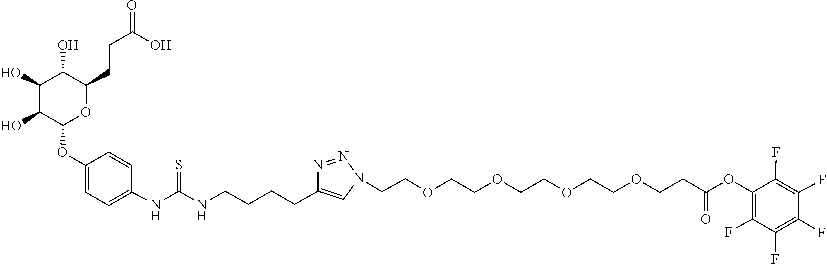 [SMILES: O=C(O)CC[C@H]1O[C@H](Oc2ccc(NC(=S)NCCCCc3cn(CCOCCOCCOCCOCCC(=O)Oc4c(F)c(F)c(F)c(F)c4F)nn3)cc2)[C@@H](O)[C@@H](O)[C@@H]1O] |
| 537 (I-66) (I-59) | 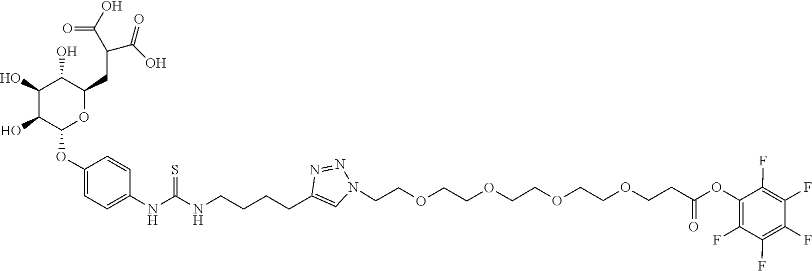 [SMILES: O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F] |
| 538 (I-64) | 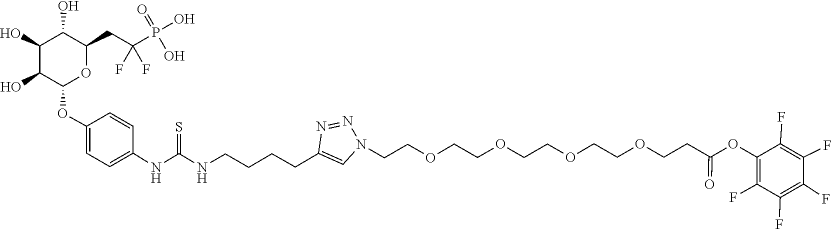 [SMILES: O=C(CCOCCOCCOCCOCCn1cc(CCCCNC(=S)Nc2ccc(O[C@H]3O[C@H](CC(F)(F)P(=O)(O)O)[C@@H](O)[C@H](O)[C@@H]3O)cc2)nn1)Oc1c(F)c(F)c(F)c(F)c1F] |
| 539 (I-65) | 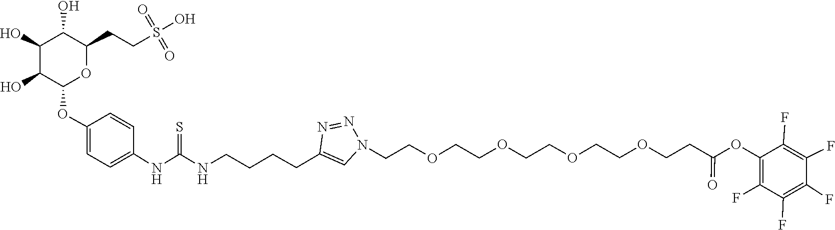 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 540 | 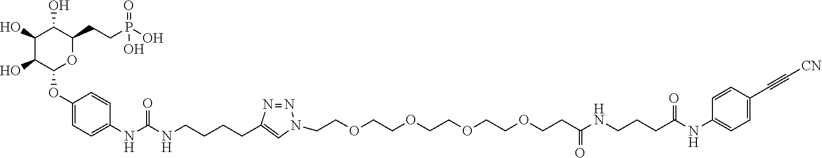 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1] |
| 541 (I-83) | 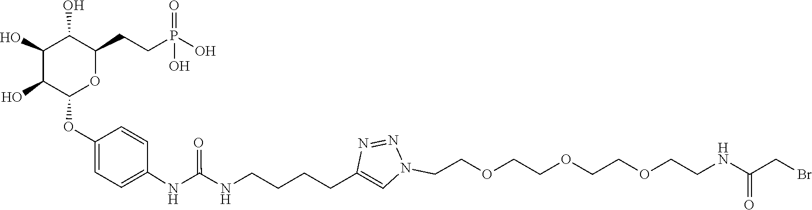 [SMILES: O=C(CBr)NCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1] |
| 542 (I-84) | 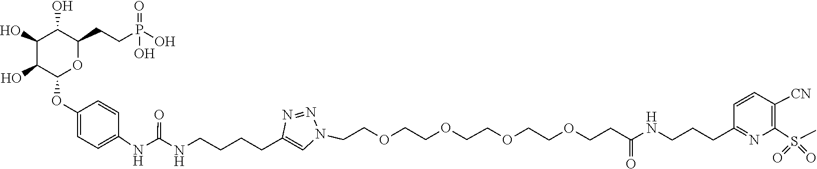 [SMILES: CS(=O)(=O)c1nc(CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)ccc1C#N] |
| 543 (I-85) | 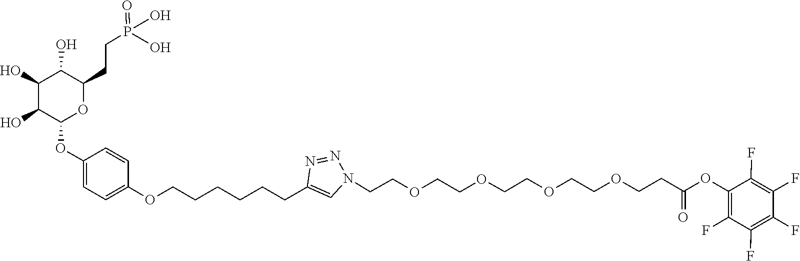 [SMILES: O=C(CCOCCOCCOCCOCCn1cc(CCCCCCOc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 544 (I-86) | 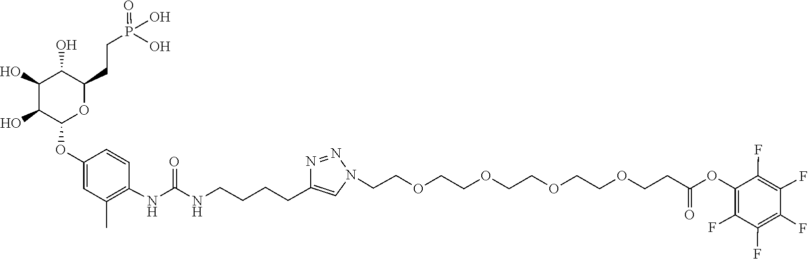 [SMILES: Cc1cc(O[C@H]2O[C@H](CCP(=O)(O)O)[C@@H](O)[C@H](O)[C@@H]2O)ccc1NC(=O)NCCCCc1cn(CCOCCOCCOCCOCCC(=O)Oc2c(F)c(F)c(F)c(F)c2F)nn1] |
| 545 (I-87) | 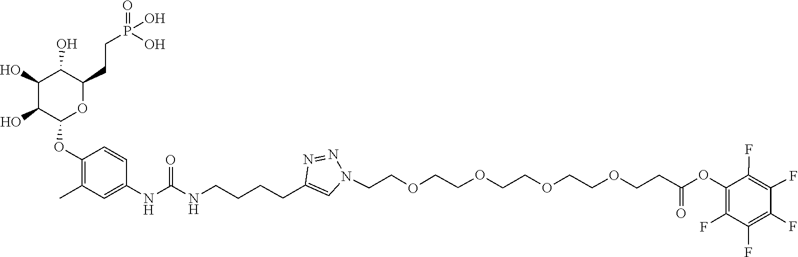 [SMILES: Cc1cc(NC(=O)NCCCCc2cn(CCOCCOCCOCCOCCC(=O)Oc3c(F)c(F)c(F)c(F)c3F)nn2)ccc1O[C@H]1O[C@H](CCP(=O)(O)O)[C@@H](O)[C@H](O)[C@@H]1O] |
| 546 (I-89) | 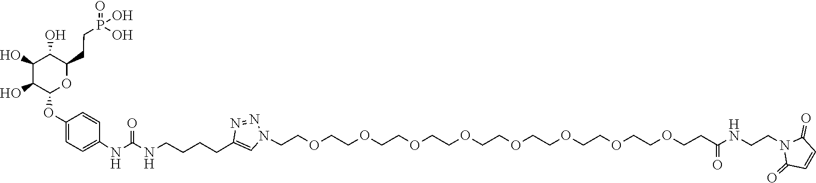 [SMILES: O=C(CCOCCOCCOCCOCCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 547 (I-90) | 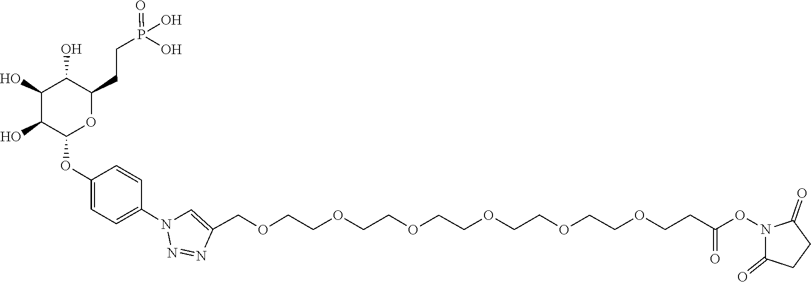 [SMILES: O=C(CCOCCOCCOCCOCCOCCOCc1cn(-c2ccc(O[C@H]3O[C@H](CCP(=O)(O)O)[C@@H](O)[C@H](O)[C@@H]3O)cc2)nn1)ON1C(=O)CCC1=O] |
| 548 (I-91) | 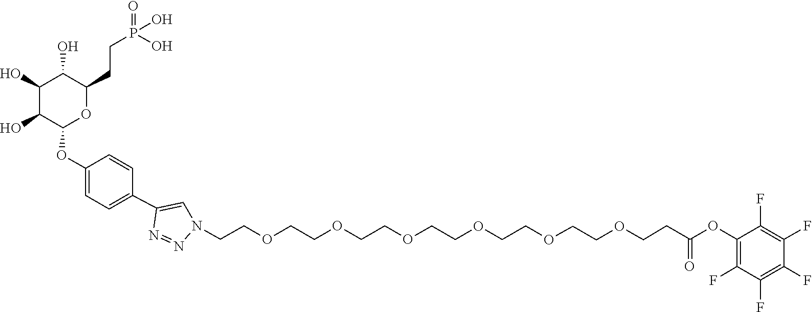 [SMILES: O=C(CCOCCOCCOCCOCCOCCOCCn1cc(-c2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 549 (I-92) | 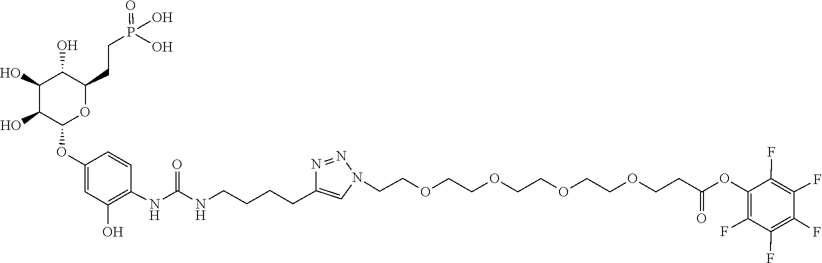 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1O] |
| 550 (I-93) | 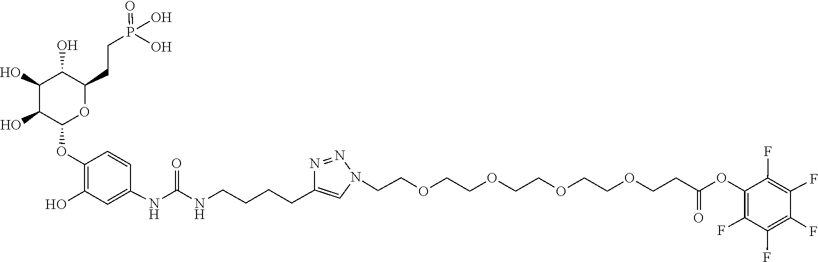 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)c(O)c1] |
| 551 (I-94) | 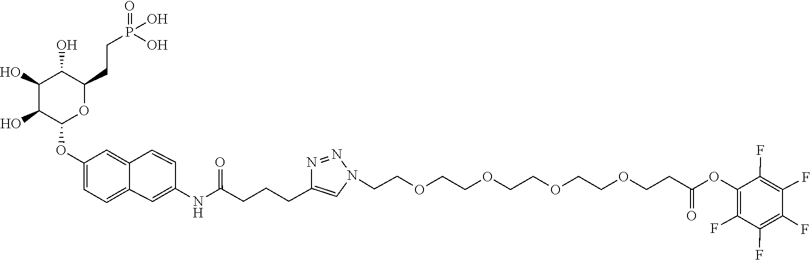 [SMILES: O=C(CCCc1cn(CCOCCOCCOCCOCCC(=O)Oc2c(F)c(F)c(F)c(F)c2F)nn1)Nc1ccc2cc(O[C@H]3O[C@H](CCP(=O)(O)O)[C@@H](O)[C@H](O)[C@@H]3O)ccc2c1] |
| 552 (I-95) | 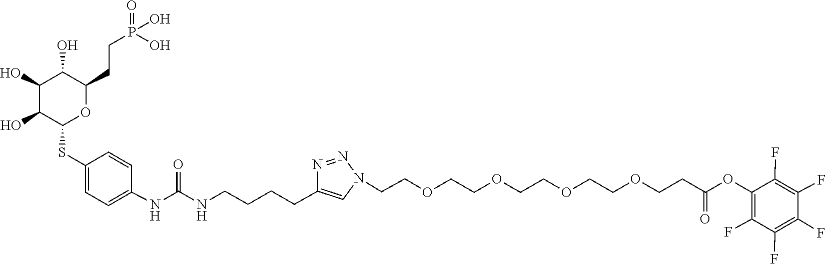 [SMILES: O=C(NCCCCc1cn(CCOCCOCCOCCOCCC(=O)Oc2c(F)c(F)c(F)c(F)c2F)nn1)Nc1ccc(S[C@H]2O[C@H](CCP(=O)(O)O)[C@@H](O)[C@H](O)[C@@H]2O)cc1] |
| 554 (I-101B) | 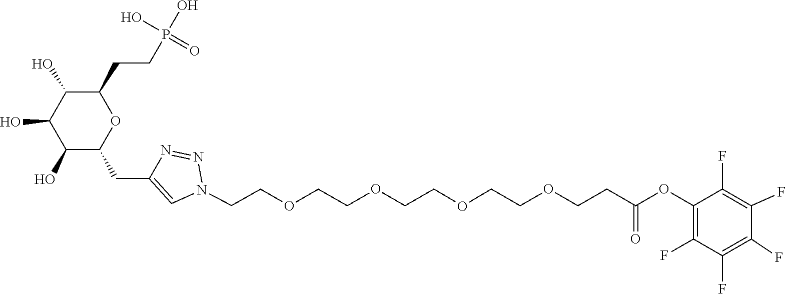 [SMILES: O=C(CCOCCOCCOCCOCCn1cc(C[C@H]2O[C@H](CCP(=O)(O)O)[C@@H](O)[C@H](O)[C@@H]2O)nn1)Oc1c(F)c(F)c(F)c(F)c1F] |
| 555 | 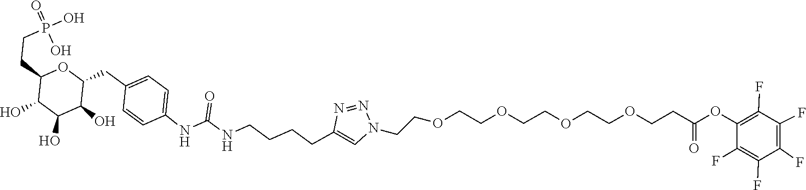 [SMILES: O=C(NCCCCc1cn(CCOCCOCCOCCOCCC(=O)Oc2c(F)c(F)c(F)c(F)c2F)nn1)Nc1ccc(C[C@H]2O[C@H](CCP(=O)(O)O)[C@@H](O)[C@H](O)[C@@H]2O)cc1] |
| 556 (I-104) | 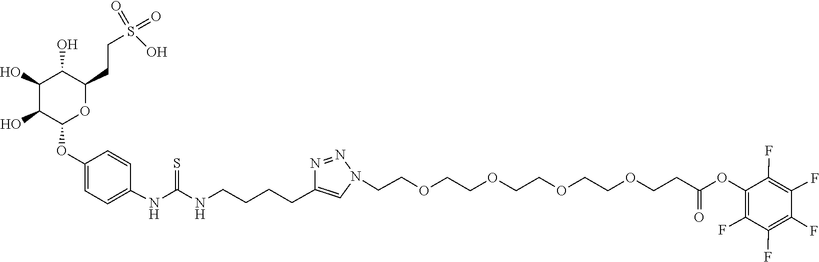 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 557 (I-103) | 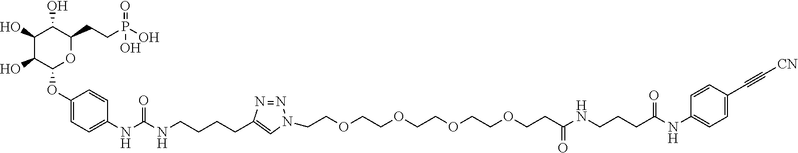 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1] |
| 558 | 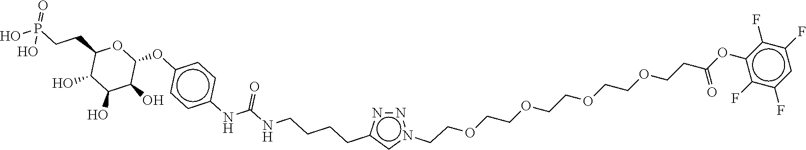 |
| 559 | 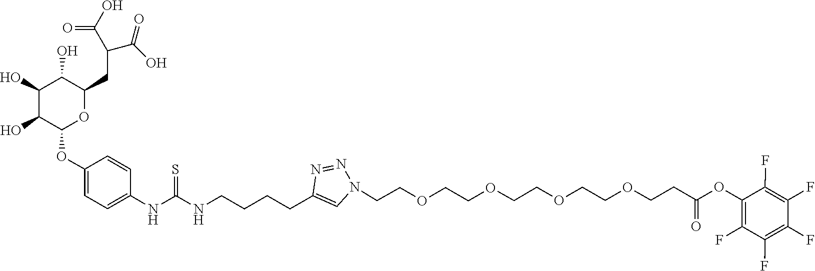 [SMILES: O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F] |
| 560 | 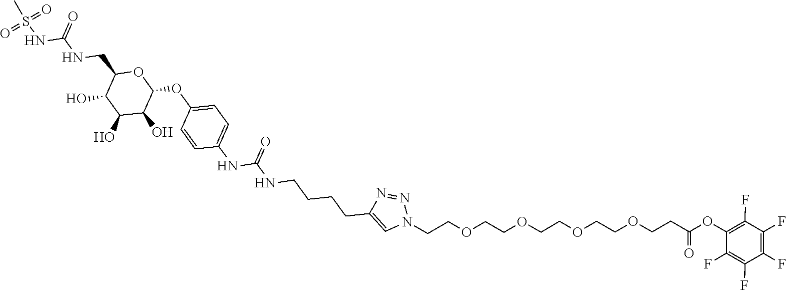 [SMILES: CS(=O)(=O)NC(=O)NC[C@H]1O[C@H](Oc2ccc(NC(=O)NCCCCc3cn(CCOCCOCCOCCOCCC(=O)Oc4c(F)c(F)c(F)c(F)c4F)nn3)cc2)[C@@H](O)[C@@H](O)[C@@H]1O] |
| 561 | 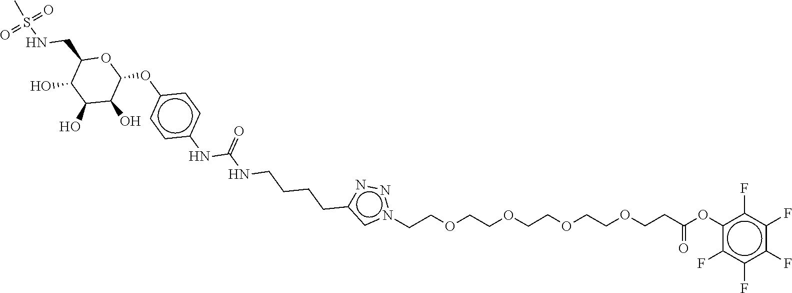 |
| 562 | 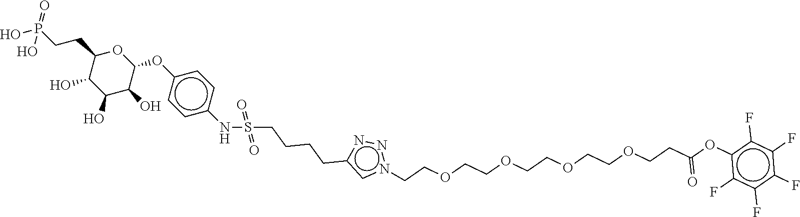 |
| 563 | 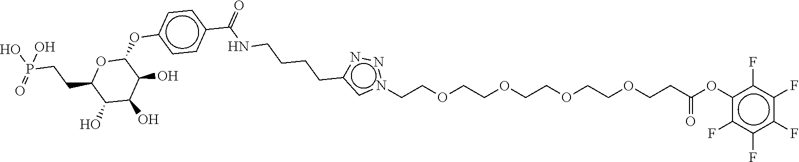 |
| 564 | 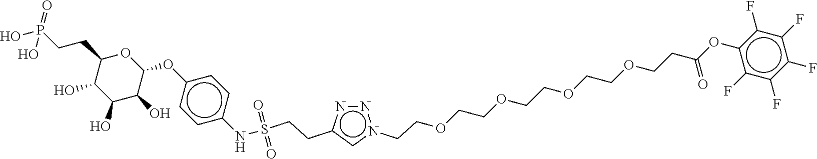 |
| 565 | 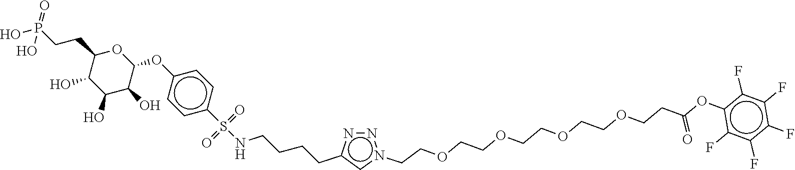 |
| 566 (I-110) | 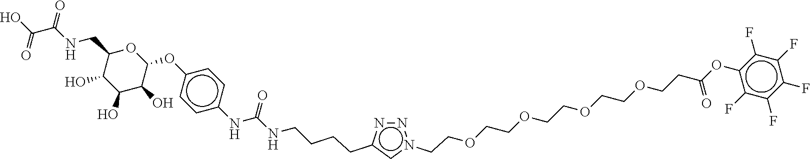 |
| 567 | 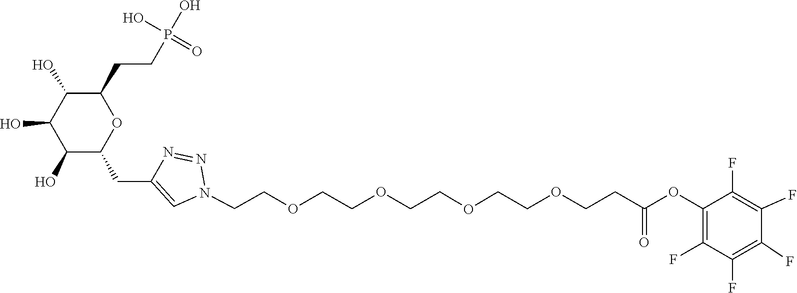 [SMILES: O=C(CCOCCOCCOCCOCCn1cc(C[C@H]2O[C@H](CCP(=O)(O)O)[C@@H](O)[C@H](O)[C@@H]2O)nn1)Oc1c(F)c(F)c(F)c(F)c1F] |
| 568 | 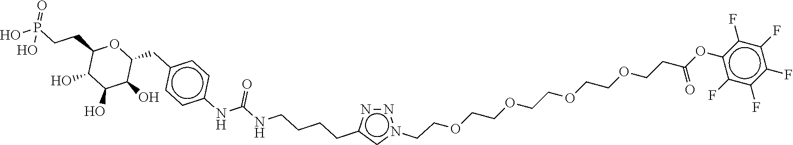 |
| 569 | 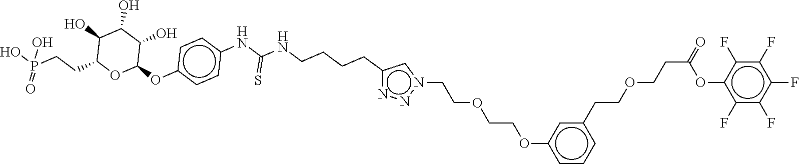 |
| 571 | 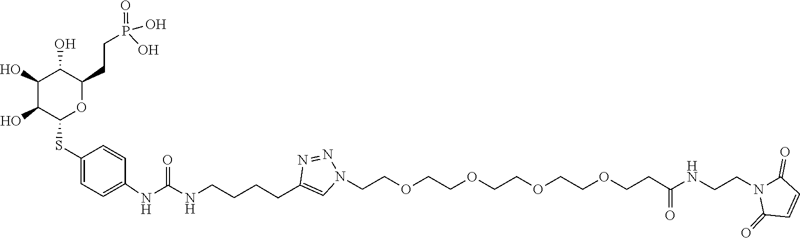 [SMILES: O=C(CCOCCOCCOCCOCCn1cc(CCCCNC(=O)Nc2ccc(S[C@H]3O[C@H](CCP(=O)(O)O)[C@@H](O)[C@H](O)[C@@H]3O)cc2)nn1)NCCN1C(=O)C=CC1=O] |
| 575 | 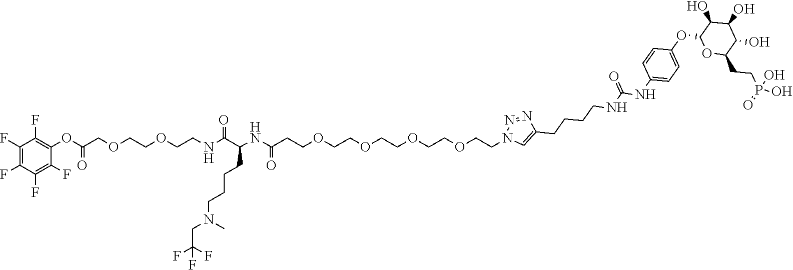 |
| 576 | 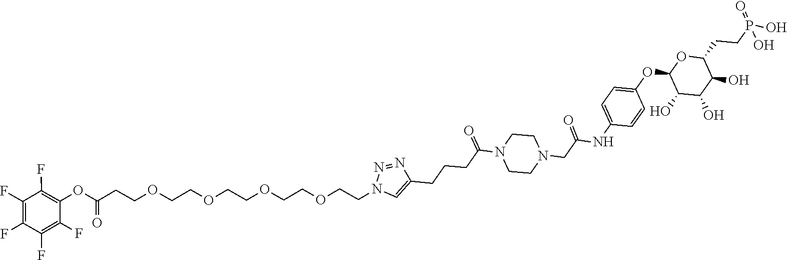 [SMILES: O=C(CN1CCN(C(=O)CCCc2cn(CCOCCOCCOCCOCCC(=O)Oc3c(F)c(F)c(F)c(F)c3F)nn2)CC1)Nc1ccc(O[C@H]2O[C@H](CCP(=O)(O)O)[C@@H](O)[C@H](O)[C@@H]2O)cc1] |

Beta configuration ligands

| | |
|---|---|
| 570 (I-106) | 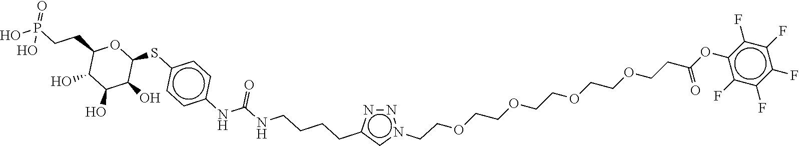 |
| 572 | 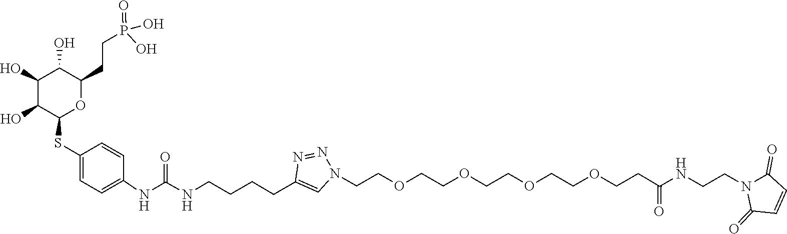 [SMILES: O=C(CCOCCOCCOCCOCCn1cc(CCCCNC(=O)Nc2ccc(S[C@@H]3O[C@H](CCP(=O)(O)O)[C@@H](O)[C@H](O)[C@@H]3O)cc2)nn1)NCCN1C(=O)C=CC1=O] |
| 573 | 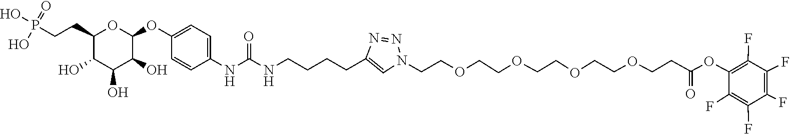 [SMILES: O=C(NCCCCc1cn(CCOCCOCCOCCOCCC(=O)Oc2c(F)c(F)c(F)c(F)c2F)nn1)Nc1ccc(O[C@@H]2O[C@H](CCP(=O)(O)O)[C@@H](O)[C@H](O)[C@@H]2O)cc1] |
| 574 | 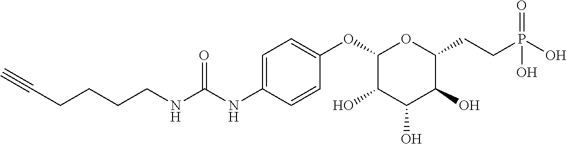 [SMILES: C#CCCCCNC(=O)Nc1ccc(O[C@@H]2O[C@H](CCP(=O)(O)O)[C@@H](O)[C@H](O)[C@@H]2O)cc1] |
| I-118A | 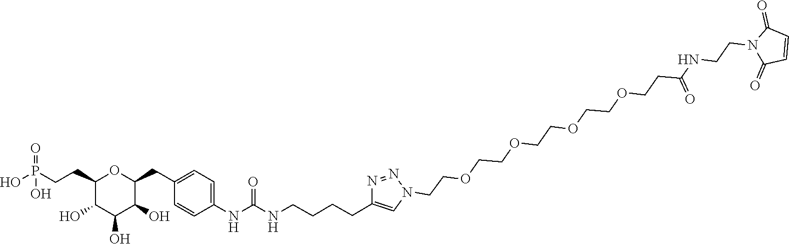 [SMILES: O=C(CCOCCOCCOCCOCCn1cc(CCCCNC(=O)Nc2ccc(C[C@@H]3O[C@H](CCP(=O)(O)O)[C@@H](O)[C@H](O)[C@@H]3O)cc2)nn1)NCCN1C(=O)C=CC1=O] |
| I-118B | 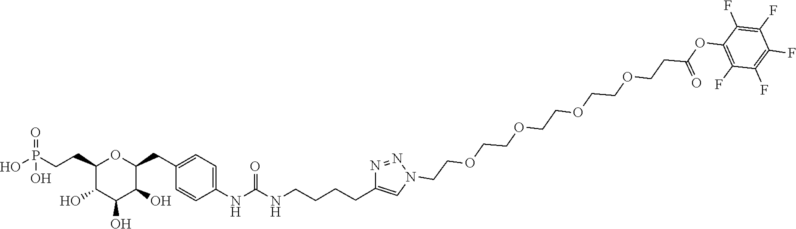 [SMILES: O=C(NCCCCc1cn(CCOCCOCCOCCOCCC(=O)Oc2c(F)c(F)c(F)c(F)c2F)nn1)Nc1ccc(C[C@@H]2O[C@H](CCP(=O)(O)O)[C@@H](O)[C@H](O)[C@@H]2O)cc1] |

TABLE 10
Other exemplary M6PR binding ligand-linkers compounds

| # | Compound Structure |
|---|---|
| 601 | 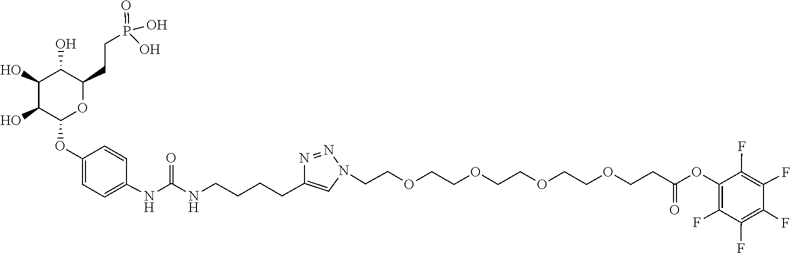 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 602 (I-8) | 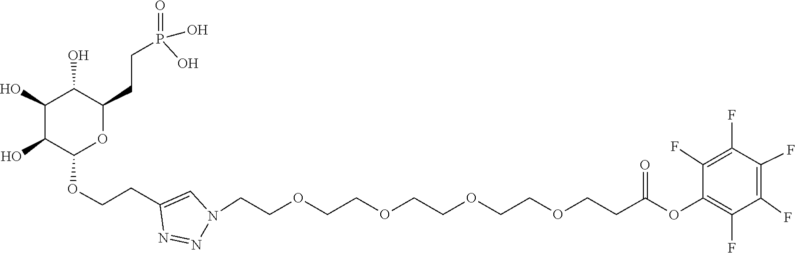 [SMILES: O=C(CCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 603 (I-9) | 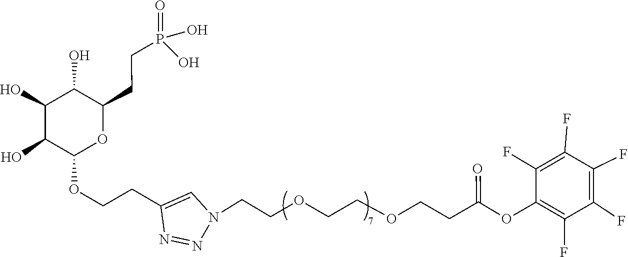 |
| 604 (I-10) | 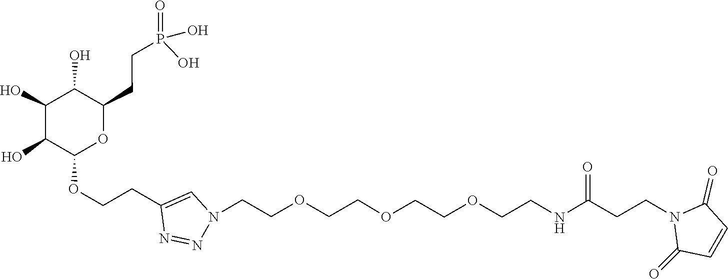 [SMILES: O=C(CCN1C(=O)C=CC1=O)NCCOCCOCCOCCn1cc(CCO[C@H]2O[C@H](CCP(=O)(O)O)[C@@H](O)[C@H](O)[C@@H]2O)nn1] |
| 605 (I-11) | 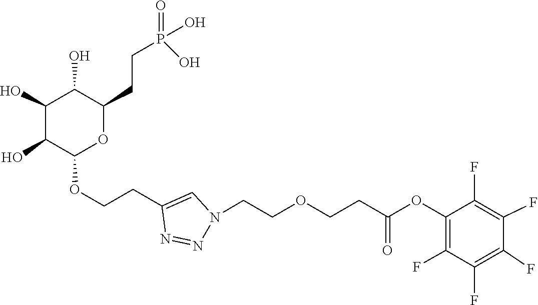 [SMILES: O=C(CCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 606 | 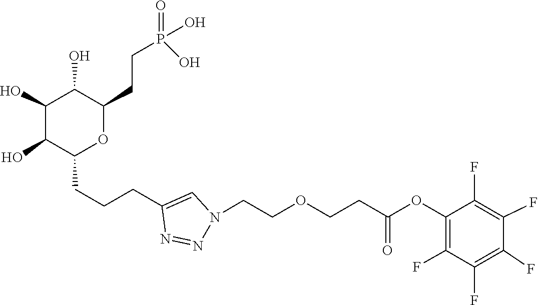 [SMILES: O=C(CCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 607 (I-13) | 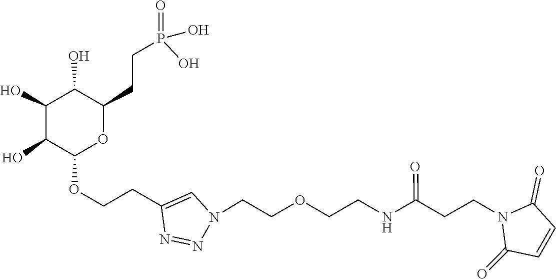 [SMILES: O=C(CCN1C(=O)C=CC1=O)NCCOCCn1cc(CCO[C@H]2O[C@H](CCP(=O)(O)O)[C@@H](O)[C@H](O)[C@@H]2O)nn1] |
| 608 (I-14) | 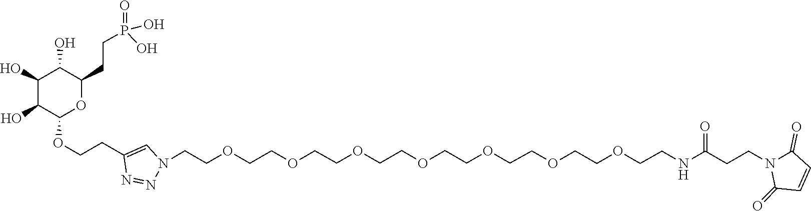 [SMILES: O=C(CCN1C(=O)C=CC1=O)NCCOCCOCCOCCOCCOCCOCCOCCn1cc(CCO[C@H]2O[C@H](CCP(=O)(O)O)[C@@H](O)[C@H](O)[C@@H]2O)nn1] |
| 609 (I-15) | 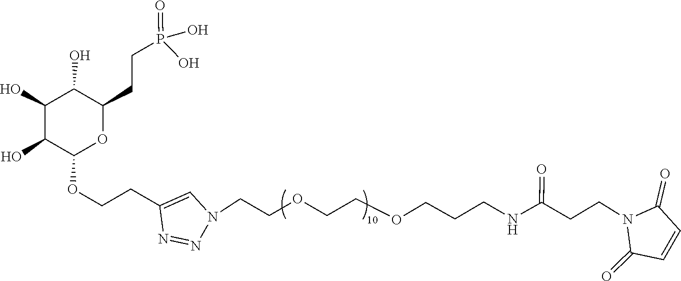 |
| 610 | 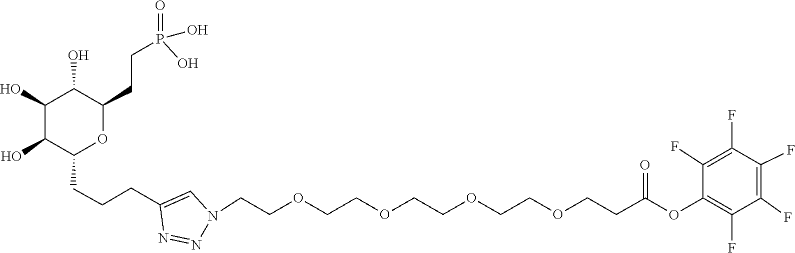 [SMILES: O=C(CCOCCOCCOCCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 611 | 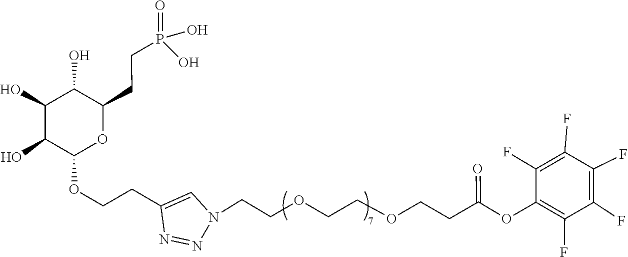 |
| 612 | 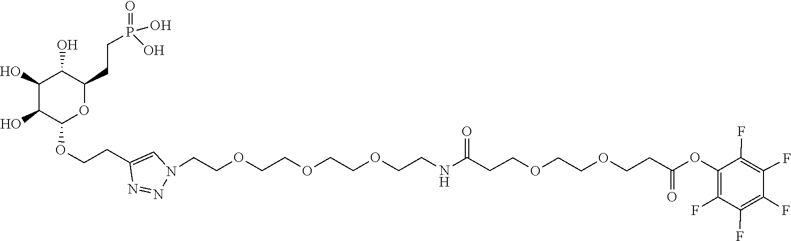 [SMILES: O=C(CCOCCOCCC(=O)Oc1c(F)c(F)c(F)c(F)c1F)NCCOCCOCCOCCn1cc(CCO[C@H]2O[C@H](CCP(=O)(O)O)[C@@H](O)[C@H](O)[C@@H]2O)nn1] |
| 613 (k = 4, l = 0) (I-33) 614 (k = 0, l = 12) (I-34) 615 (k = 2, l = 6) (I-35) | 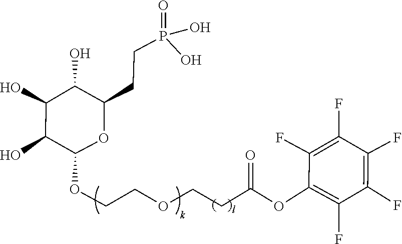 |
| 616 | 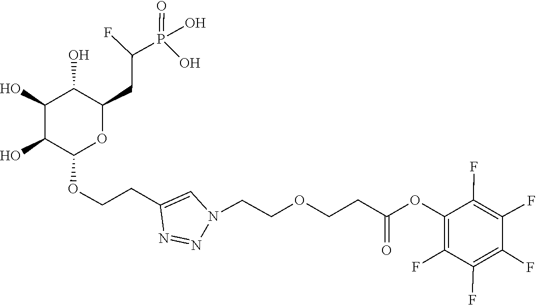 [SMILES: O=C(CCOCCn1cc(CCO[C@H]2O[C@H](CC(F)P(=O)(O)O)[C@@H](O)[C@H](O)[C@@H]2O)nn1)Oc1c(F)c(F)c(F)c(F)c1F] |
| 617 | 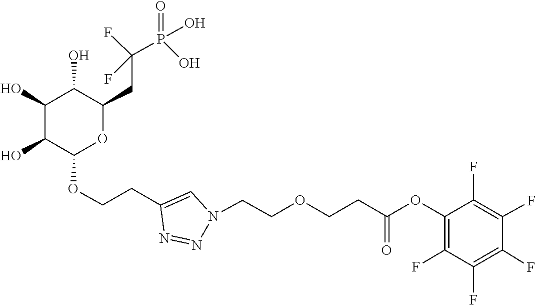 [SMILES: O=C(CCOCCn1cc(CCO[C@H]2O[C@H](CC(F)(F)P(=O)(O)O)[C@@H](O)[C@H](O)[C@@H]2O)nn1)Oc1c(F)c(F)c(F)c(F)c1F] |
| 618 | 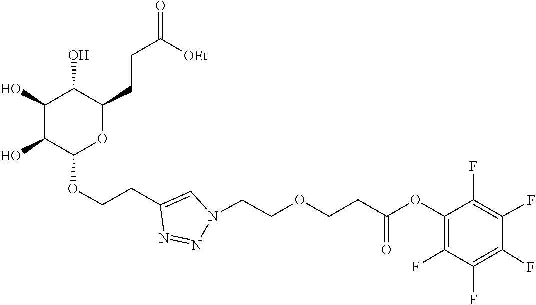 [SMILES: CCOC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 619 | 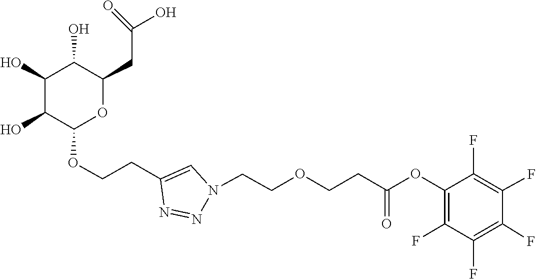 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 620 | 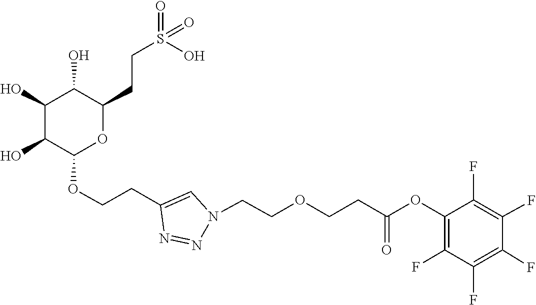 [SMILES: O=C(CCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 621 | 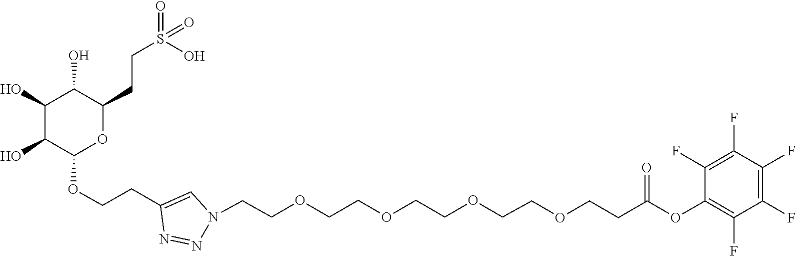 [SMILES: O=C(CCOCCOCCOCCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 622 | 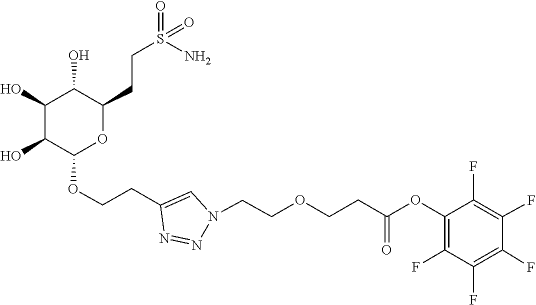 [SMILES: NS(=O)(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 623 | 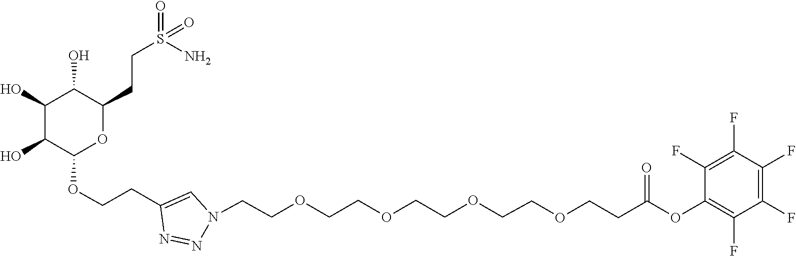 [SMILES: NS(=O)(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 624 | 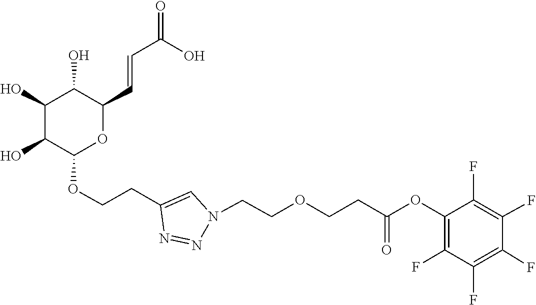 [SMILES: O=C(O)/C=C/[C@H]1O[C@H](OCCc2cn(CCOCCC(=O)Oc3c(F)c(F)c(F)c(F)c3F)nn2)[C@@H](O)[C@@H](O)[C@@H]1O] |
| 625 | 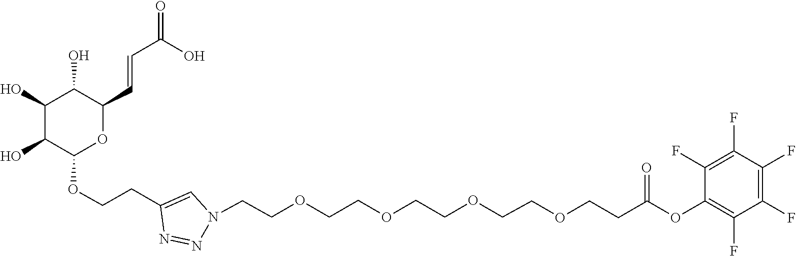 [SMILES: O=C(O)/C=C/[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 626 | 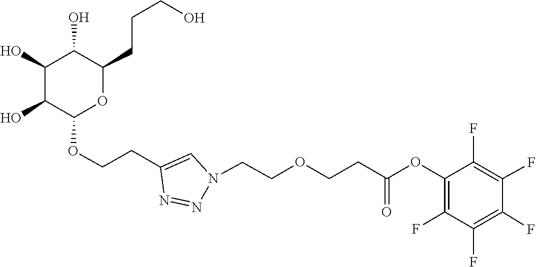 [SMILES: O=C(CCOCCn1cc(CCO[C@H]2O[C@H](CCCO)[C@@H](O)[C@H](O)[C@@H]2O)nn1)Oc1c(F)c(F)c(F)c(F)c1F] |
| 627 | 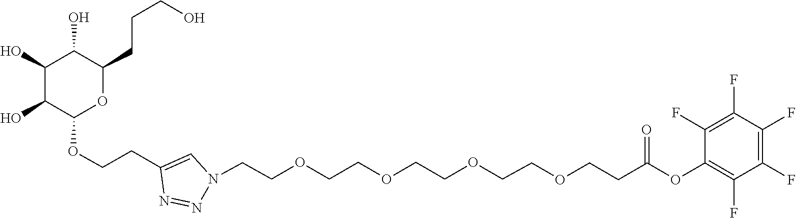 [SMILES: O=C(CCOCCOCCOCCOCCn1cc(CCO[C@H]2O[C@H](CCCO)[C@@H](O)[C@H](O)[C@@H]2O)nn1)Oc1c(F)c(F)c(F)c(F)c1F] |
| 628 | 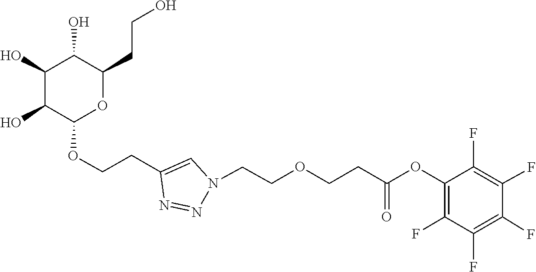 [SMILES: O=C(CCOCCn1cc(CCO[C@H]2O[C@H](CCO)[C@@H](O)[C@H](O)[C@@H]2O)nn1)Oc1c(F)c(F)c(F)c(F)c1F] |
| 629 | 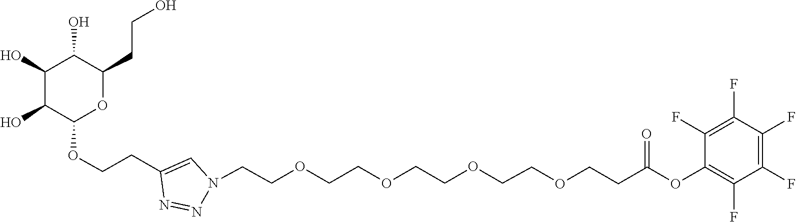 [SMILES: O=C(CCOCCOCCOCCOCCn1cc(CCO[C@H]2O[C@H](CCO)[C@@H](O)[C@H](O)[C@@H]2O)nn1)Oc1c(F)c(F)c(F)c(F)c1F] |
| 630 | 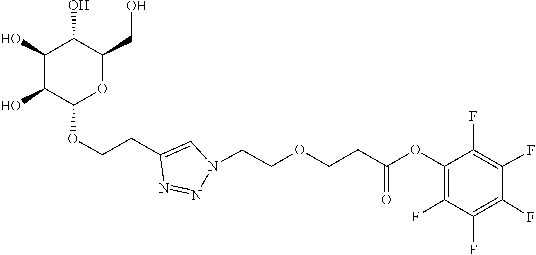 [SMILES: O=C(CCOCCn1cc(CCO[C@H]2O[C@H](CO)[C@@H](O)[C@H](O)[C@@H]2O)nn1)Oc1c(F)c(F)c(F)c(F)c1F] |
| 631 | 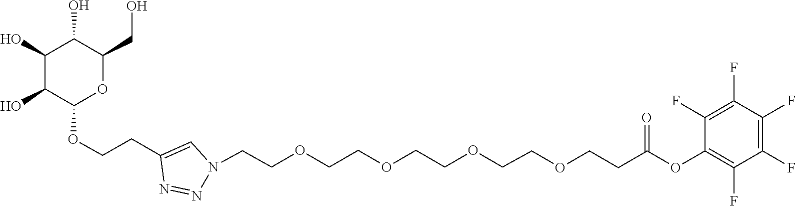 [SMILES: O=C(CCOCCOCCOCCOCCn1cc(CCO[C@H]2O[C@H](CO)[C@@H](O)[C@H](O)[C@@H]2O)nn1)Oc1c(F)c(F)c(F)c(F)c1F] |
| 632 | 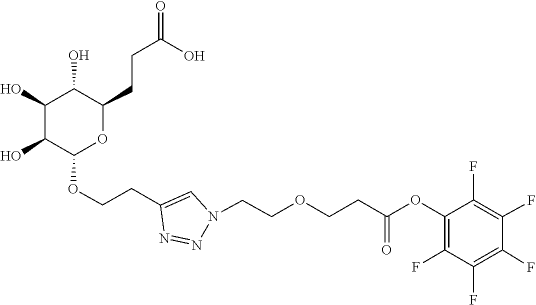 [SMILES: O=C(O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 633 | 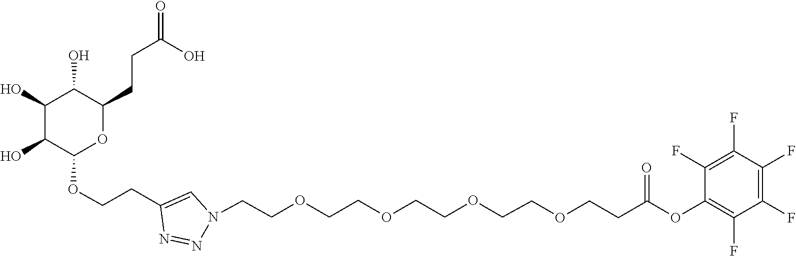 [SMILES: O=C(O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 634 | 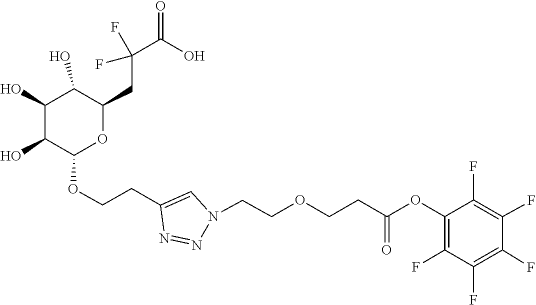 [SMILES: O=C(CCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 635 | 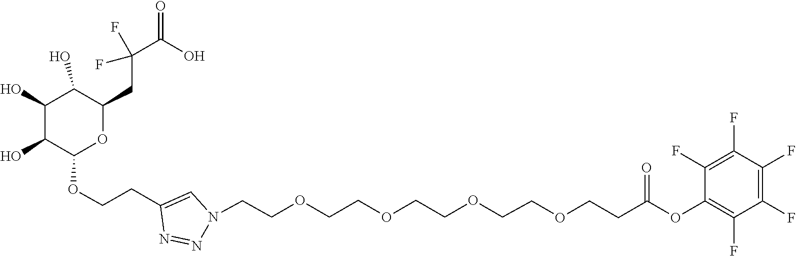 [SMILES: O=C(CCOCCOCCOCCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 636 | 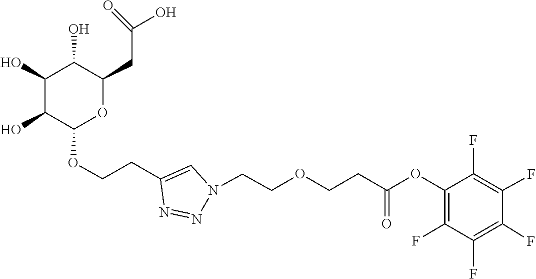 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 637 | 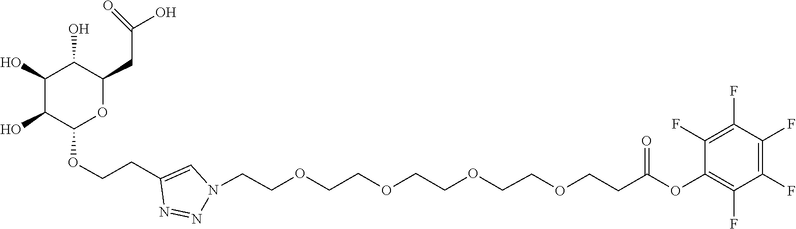 [SMILES: O=C(O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 638 | 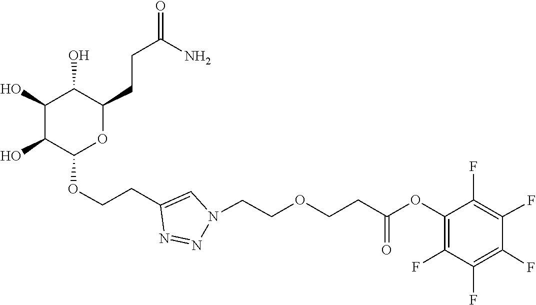 [SMILES: NC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 639 | 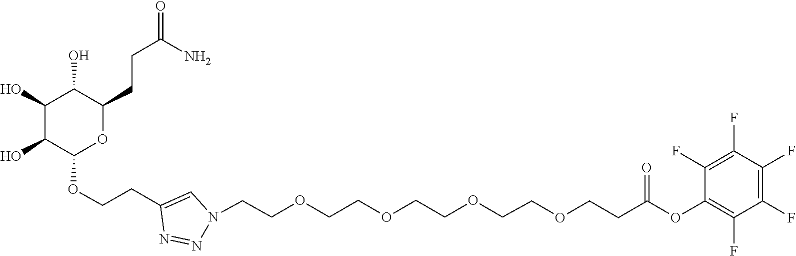 [SMILES: NC(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 640 | 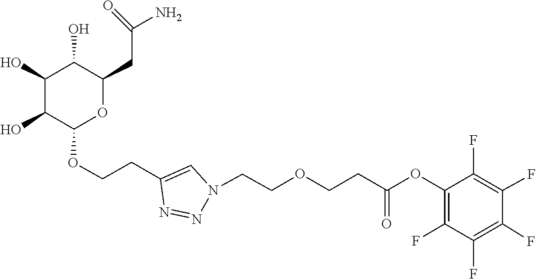 [SMILES: NC(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 641 | 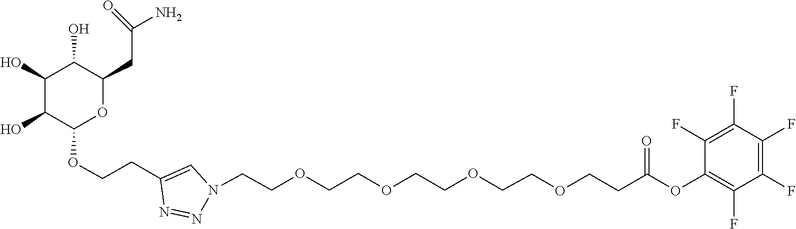 [SMILES: NC(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 642 | 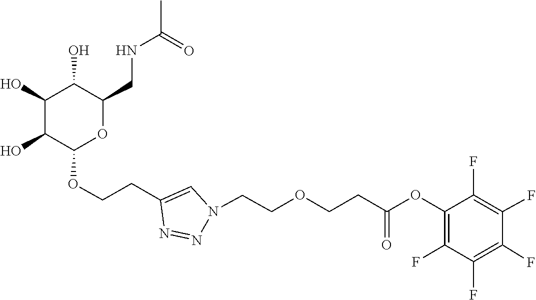 [SMILES: CC(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 643 | 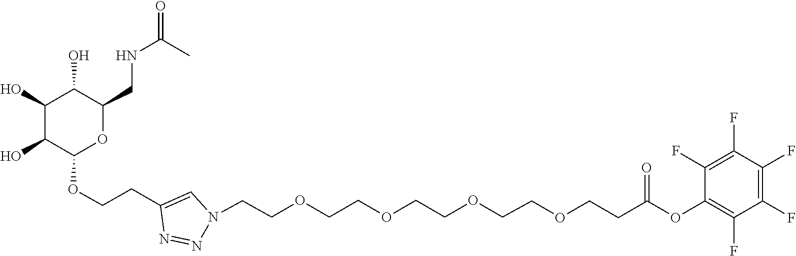 [SMILES: CC(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 644 | 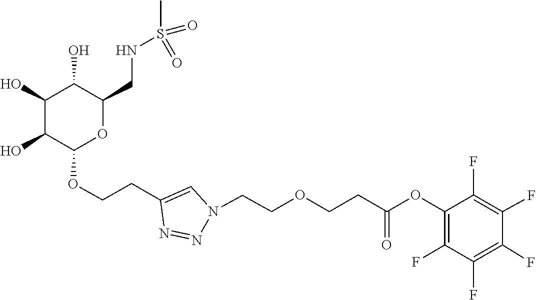 [SMILES: CS(=O)(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 645 | 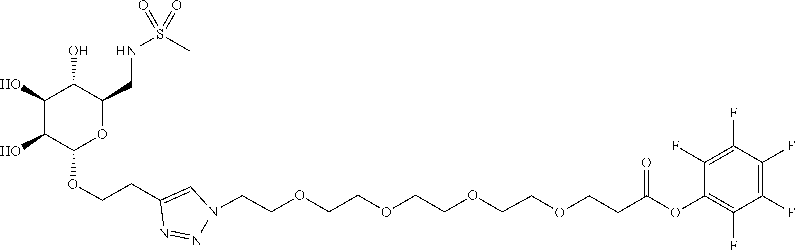 [SMILES: CS(=O)(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 646 | 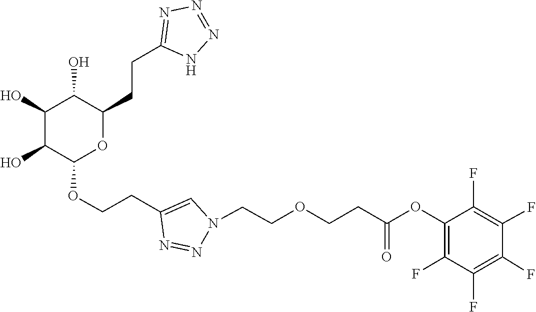 [SMILES: O=C(CCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 647 | 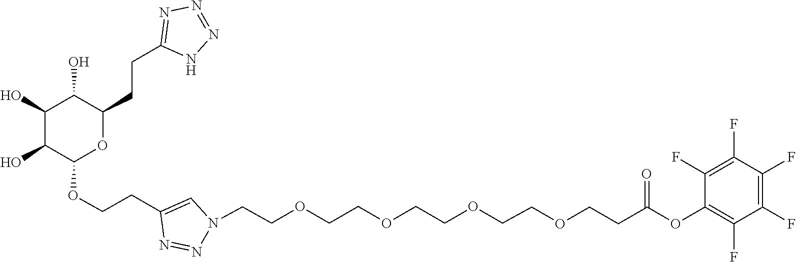 [SMILES: O=C(CCOCCOCCOCCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 648 | 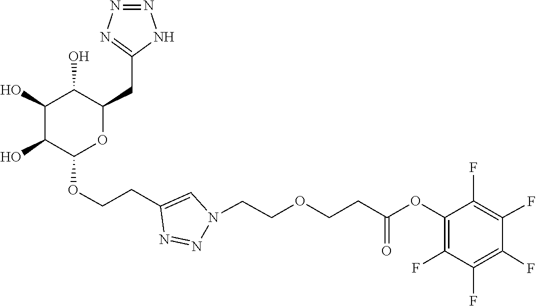 [SMILES: O=C(CCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 649 | 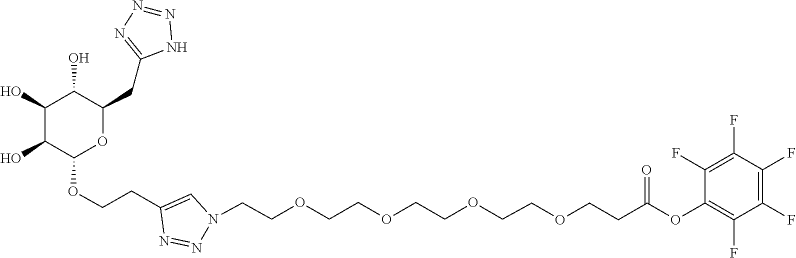 [SMILES: O=C(CCOCCOCCOCCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 650 | 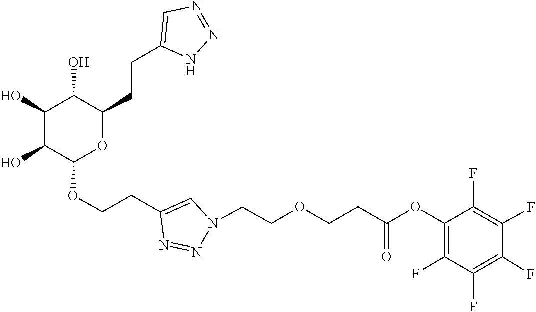 [SMILES: O=C(CCOCCn1cc(CCO[C@H]2O[C@H](CCc3cnn[nH]3)[C@@H](O)[C@H](O)[C@@H]2O)nn1)Oc1c(F)c(F)c(F)c(F)c1F] |
| 651 | 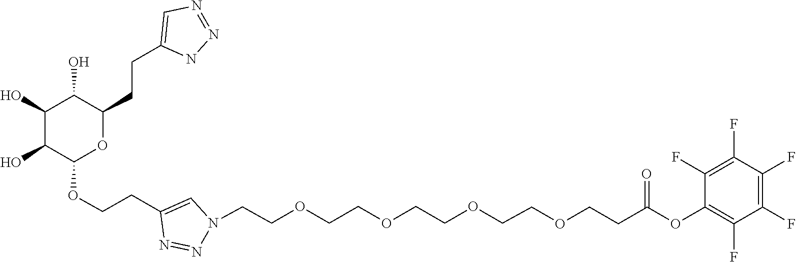 [SMILES: O=C(CCOCCOCCOCCOCCn1cc(CCO[C@H]2O[C@H](CCc3cnn[nH]3)[C@@H](O)[C@H](O)[C@@H]2O)nn1)Oc1c(F)c(F)c(F)c(F)c1F] |
| 652 | 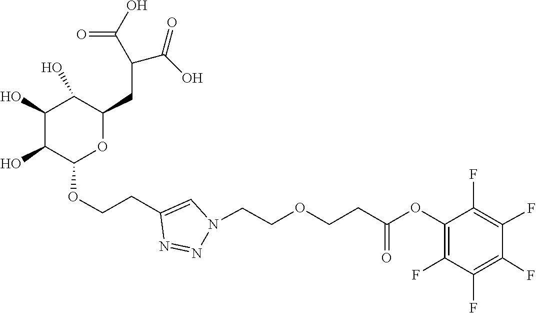 [SMILES: O=C(CCOCCn1cc(CCO[C@H]2O[C@H](CC(C(=O)O)C(=O)O)[C@@H](O)[C@H](O)[C@@H]2O)nn1)Oc1c(F)c(F)c(F)c(F)c1F] |
| 653 | 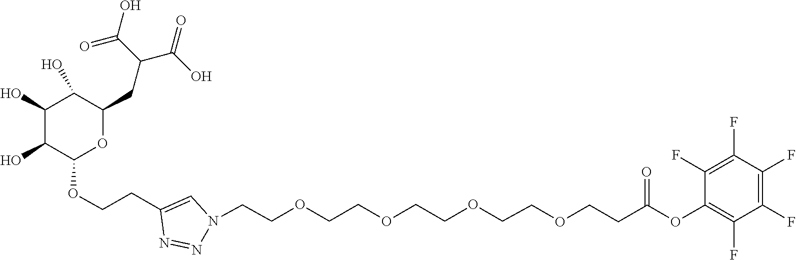 [SMILES: O=C(CCOCCOCCOCCOCCn1cc(CCO[C@H]2O[C@H](CC(C(=O)O)C(=O)O)[C@@H](O)[C@H](O)[C@@H]2O)nn1)Oc1c(F)c(F)c(F)c(F)c1F] |
| 654 | 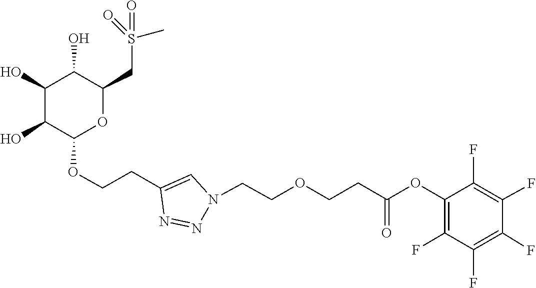 [SMILES: CS(=O)(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 655 | 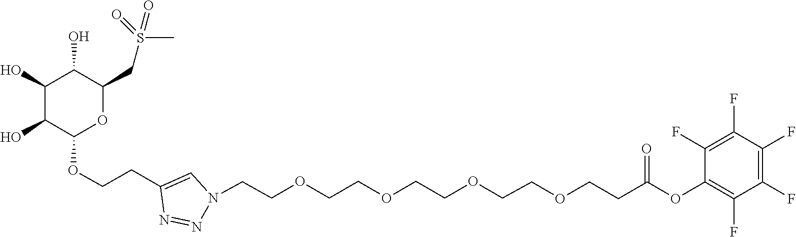 [SMILES: CS(=O)(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 656 (I-37) | 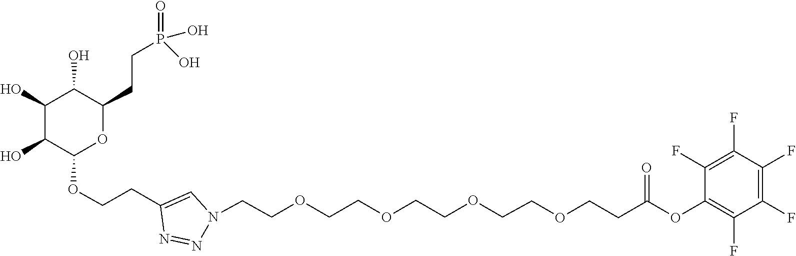 [SMILES: O=C(CCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 657 | 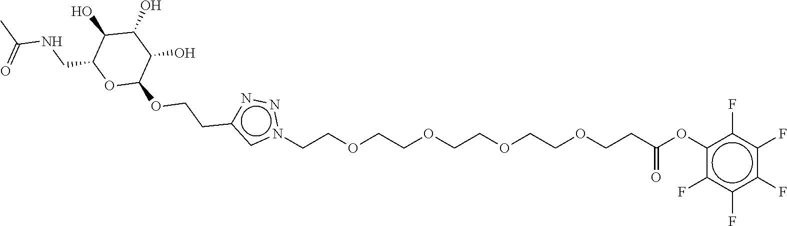 |
| 658 | 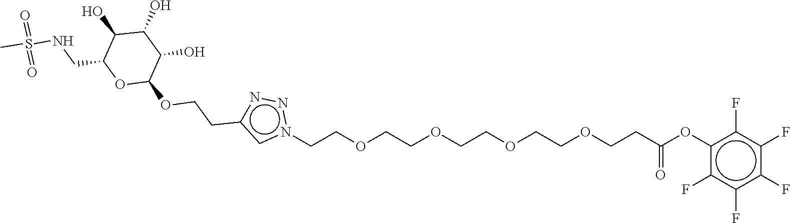 |
| 659 (I-107) | 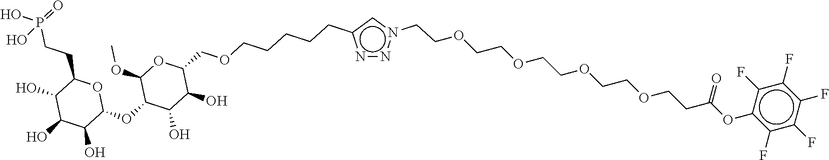 |
| 660 | 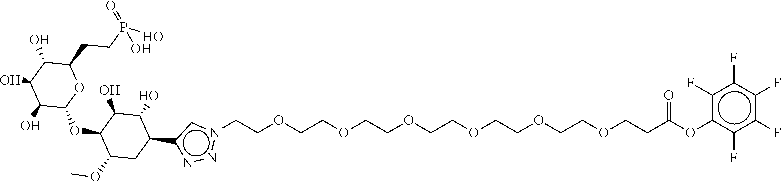 |

TABLE 11
Reference Compounds

| # | Compound Structure |
|---|---|
| 661 (L- mann.) | 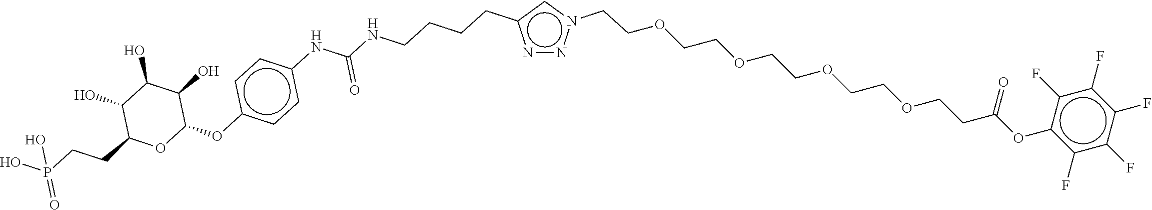 |
| 662 | 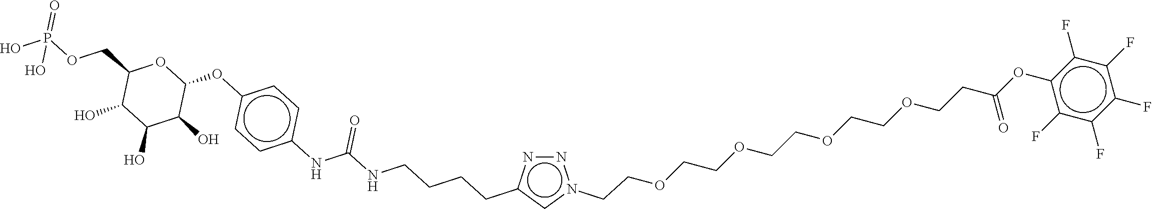 |
| 663 | 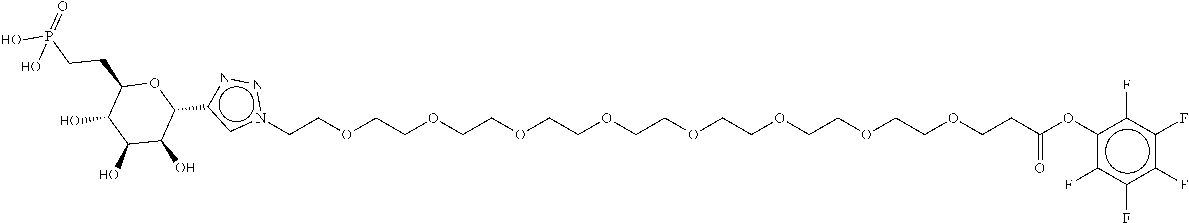 |
| 664 | 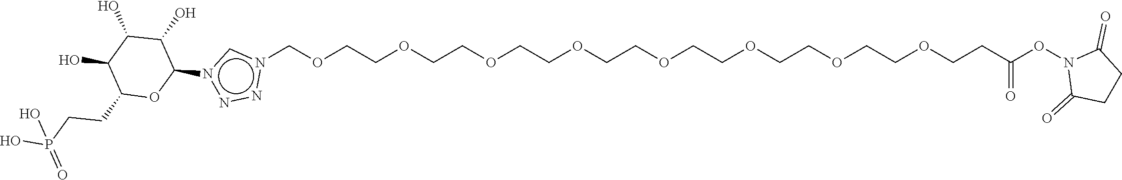 |
| 665 | 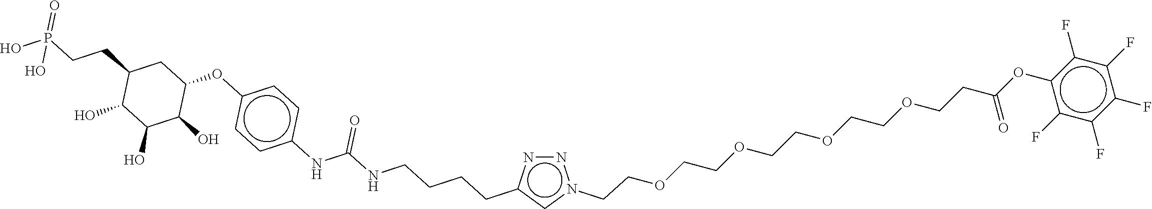 |
| 666 | 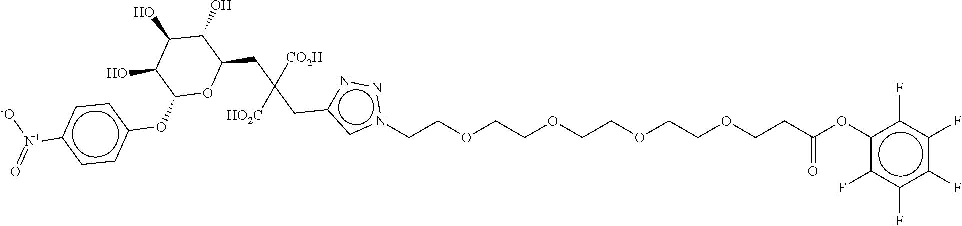 |

The structures of exemplary multivalent (n>1) compounds of this disclosure are shown in Table 12 which can be used to prepare conjugates of moieties of interest.

TABLE 12
Multivalent M6PR binding compounds having chemoselective ligation group

| # | Structure |
|---|---|
| 701 | 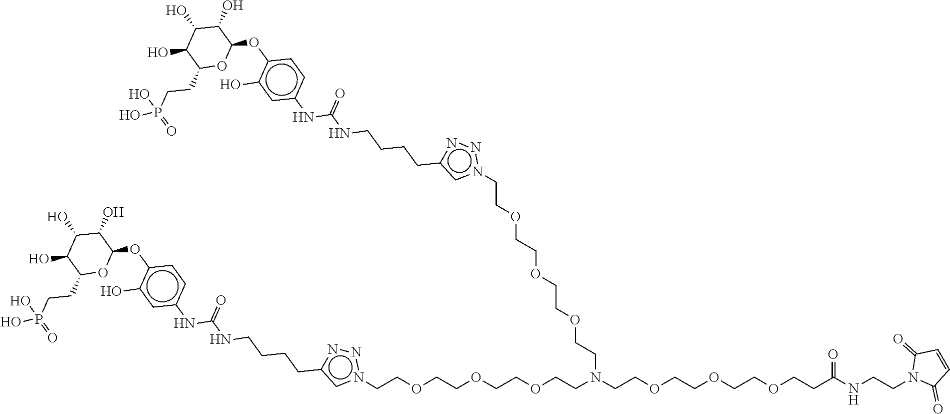 |
| 702 | 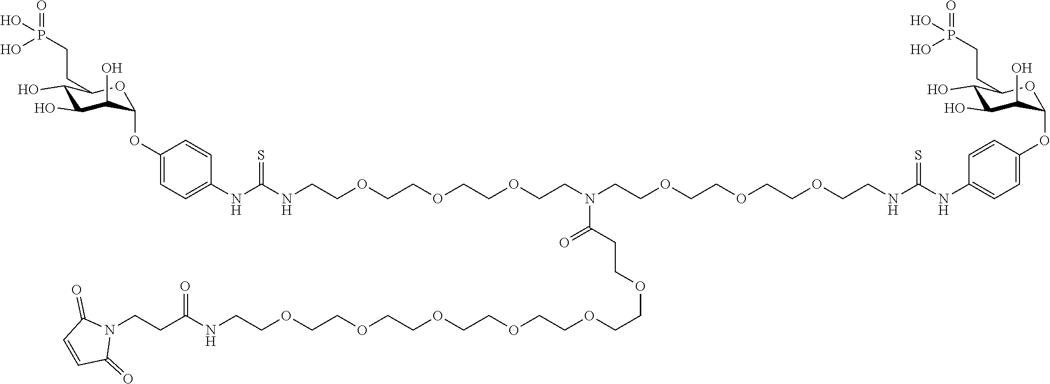 |
| 703 | 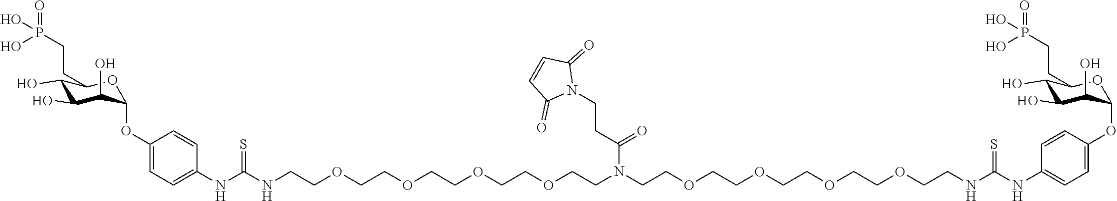 |
| 704 (I-40) | 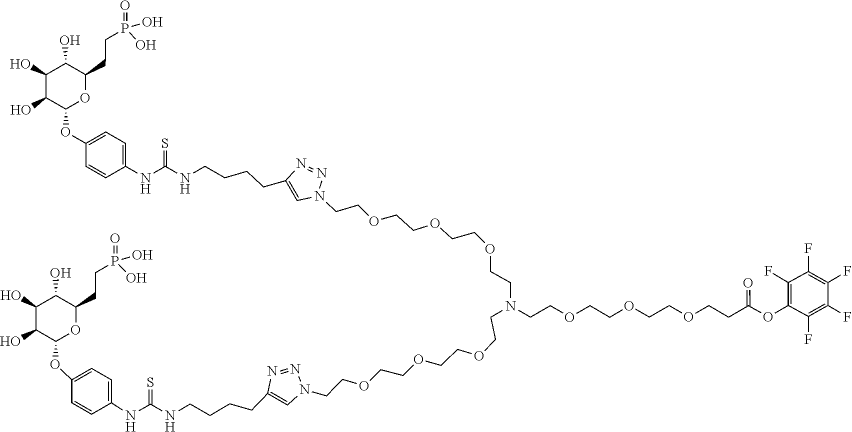 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 705 (I-41) | 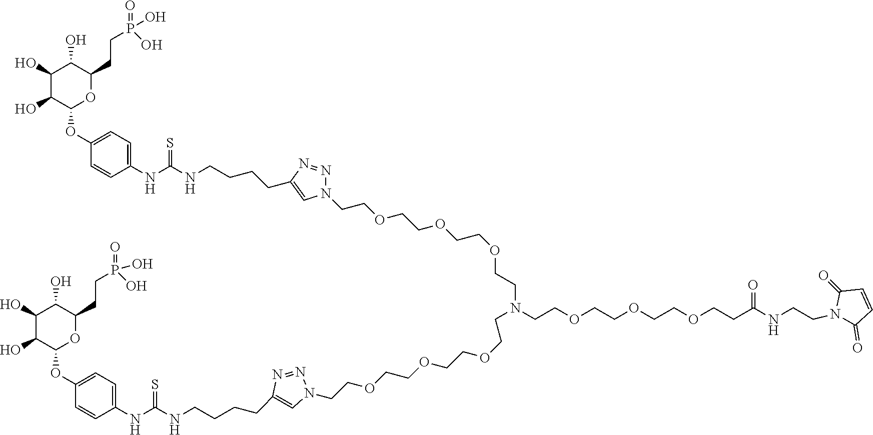 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 706 (I-43) | 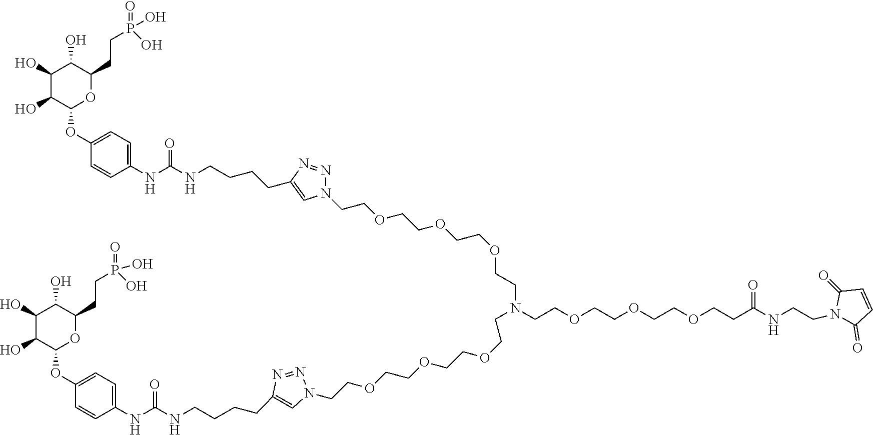 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 707 (I-58) | 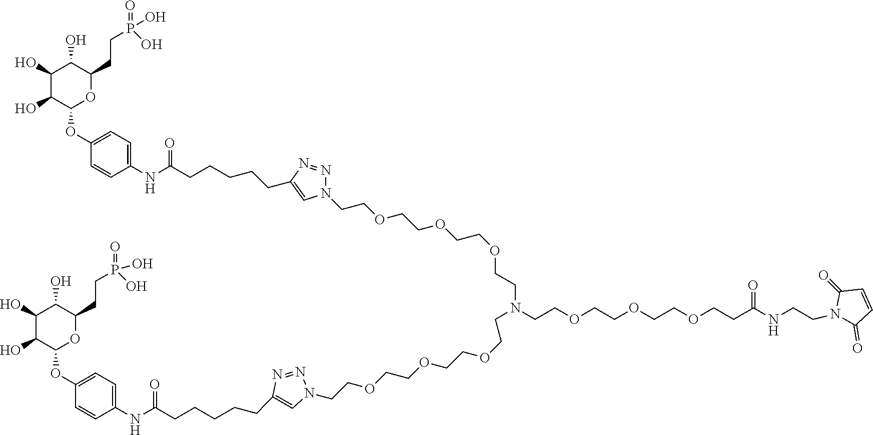 [SMILES: O=C(CCOCCOCCOCCN(CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 708 (I-42) | 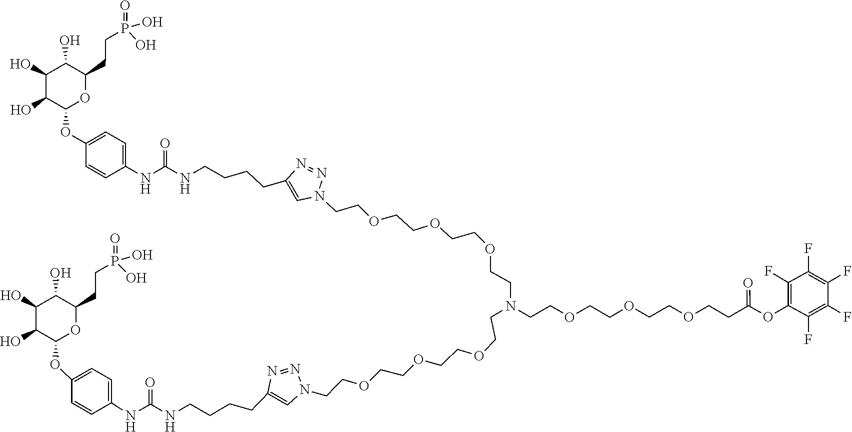 [SMILES: O=C(NCCCCc1cn(CCOCCOCCOCCN(CCOCCOCCOCCC(=O)Oc2c(F)c(F)c(F)c(F)c2F)CCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)nn1)Nc1ccc(O[C@H]2O[C@H](CCP(=O)(O)O)[C@@H](O)[C@H](O)[C@@H]2O)cc1] |
| 709 (I-53) | 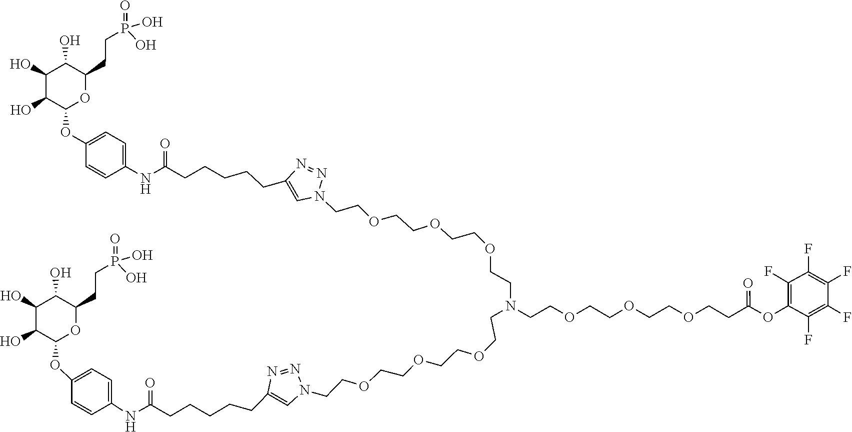 [SMILES: O=C(CCCCCc1cn(CCOCCOCCOCCN(CCOCCOCCOCCC(=O)Oc2c(F)c(F)c(F)c(F)c2F)CCOCCOCCOCCn2cc(CCCCCC(=O)Nc3ccc(O[C@H]4O[C@H](CCP(=O)(O)O)[C@@H](O)[C@H](O)[C@@H]4O)cc3)nn2)nn1)Nc1ccc(O[C@H]2O[C@H](CCP(=O)(O)O)[C@@H](O)[C@H](O)[C@@H]2O)cc1] |
| 710 (I-96) | 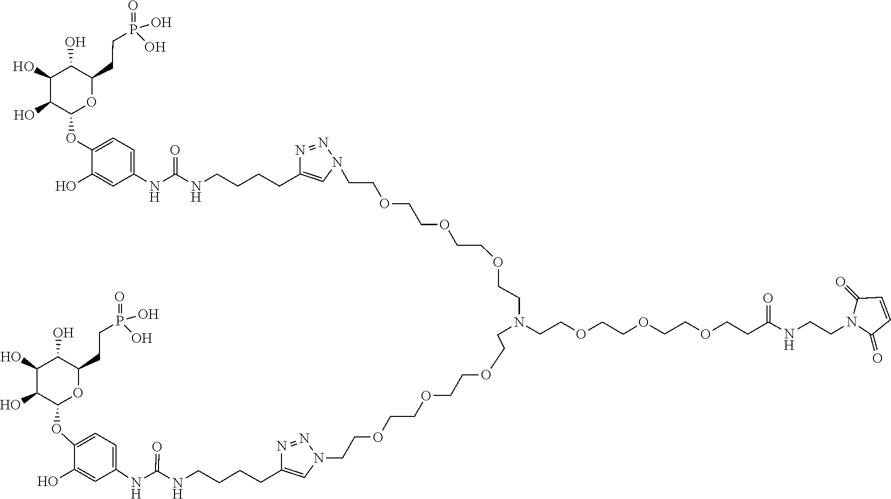 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)NCCN1C(=O)C=CC1=O] |
| 711 | 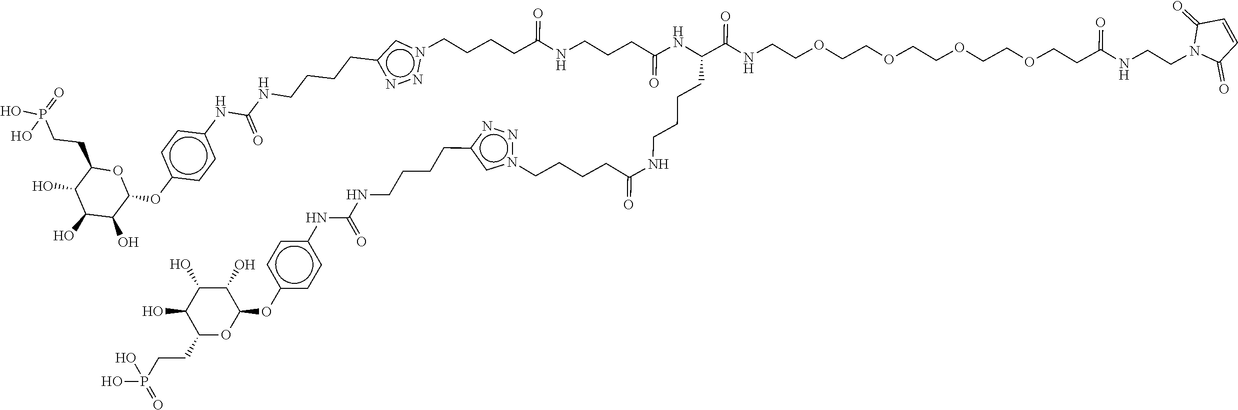 |
| 712 | 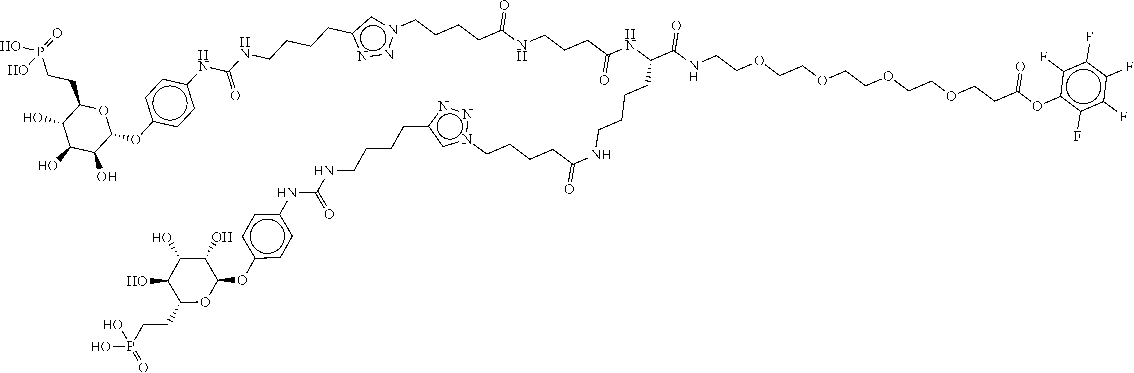 |
| 713 | 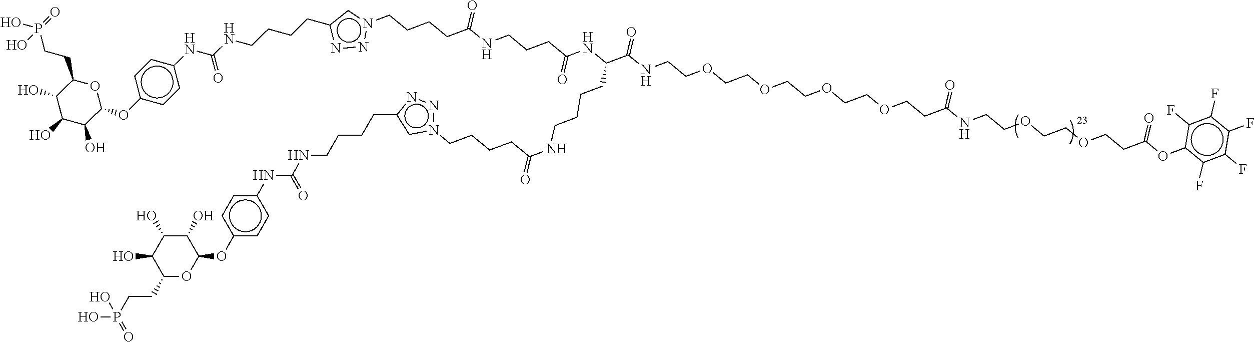 |
| 714 | 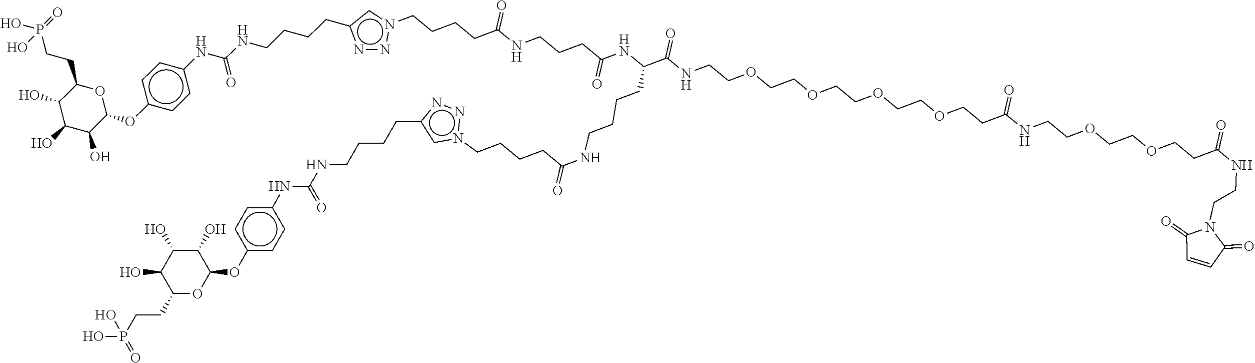 |
| 715 | 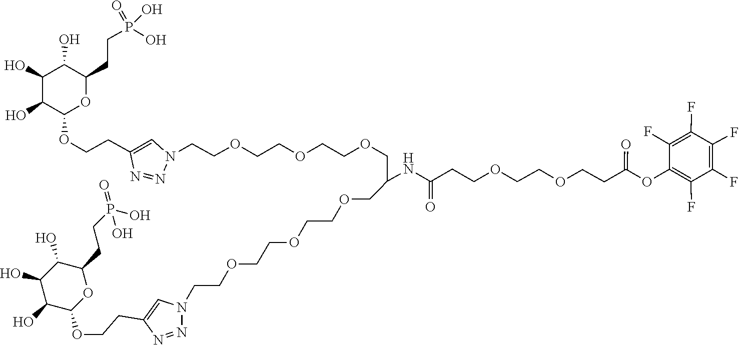 [SMILES: O=C(CCOCCOCCC(=O)Oc1c(F)c(F)c(F)c(F)c1F)NC(COCCOCCOCCn1cc(CCO[C@H]2O[C@H](CCP(=O)(O)O)[C@@H](O)[C@H](O)[C@@H]2O)nn1)COCCOCCOCCn1cc(CCO[C@H]2O[C@H](CCP(=O)(O)O)[C@@H](O)[C@H](O)[C@@H]2O)nn1] |
| 716 (I-12) | 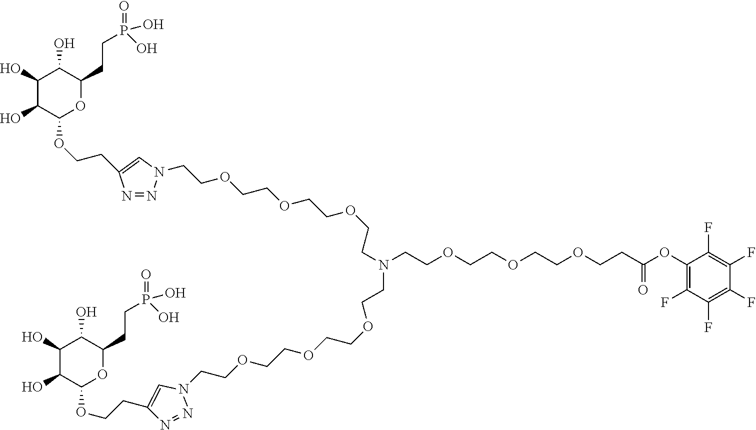 [SMILES: O=C(CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |

Trimeric ligands

| | |
|---|---|
| 717 (I-44) | 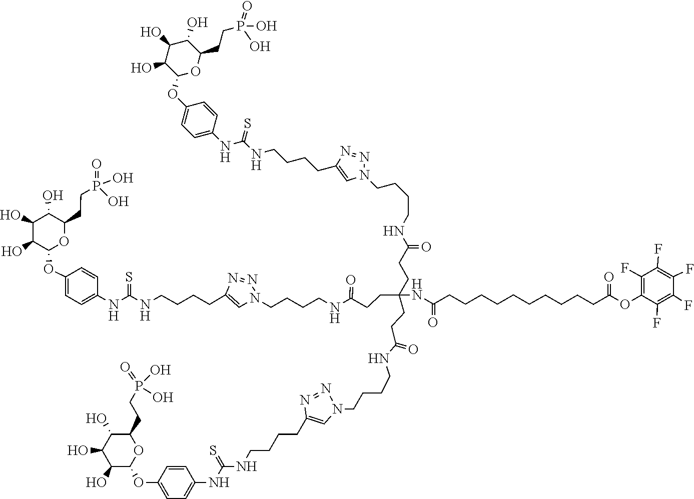 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCCCCCCCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 718 (I-45) | 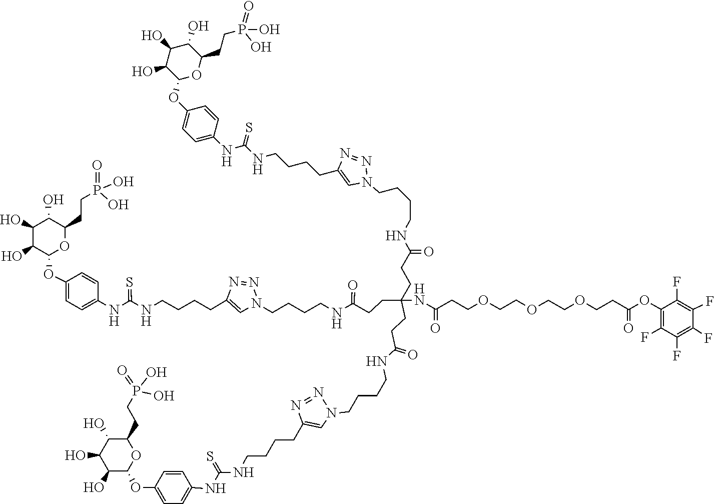 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCOCCOCCOCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 719 (I-54) | 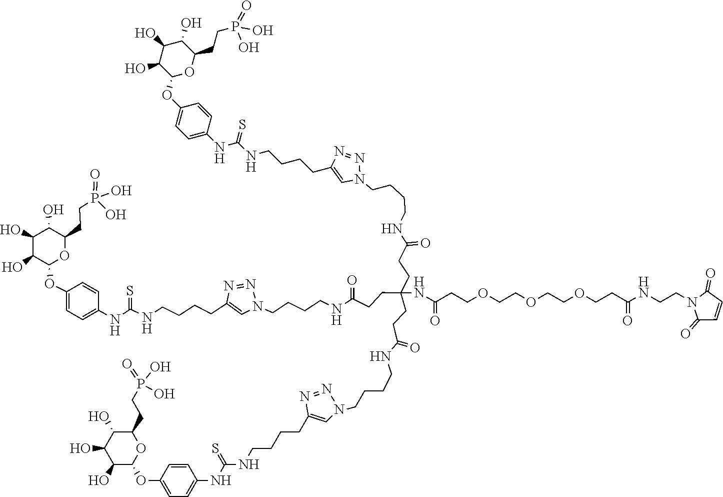 [SMILES: O=C(CCOCCOCCOCCC(=O)NC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 720 (I-81) | 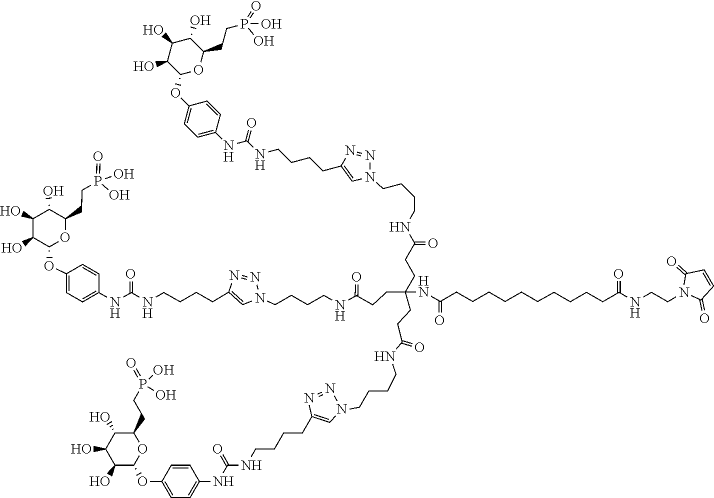 [SMILES: O=C(CCCCCCCCCCC(=O)NC(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 721 (I-46) | 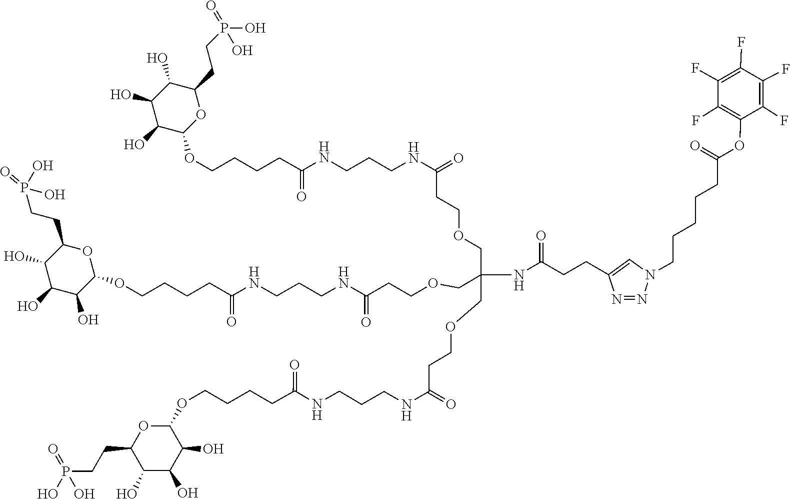 [SMILES: O=C(CCCCO[C@H]1O[C@H](CCP(=O)(O)O)[C@@H](O)[C@H](O)[C@@H]1O)NCCCNC(=O)CCOCC(COCCC(=O)NCCCNC(=O)CCCCO[C@H]1O[C@H](CCP(=O)(O)O)[C@@H](O)[C@H](O)[C@@H]1O)(COCCC(=O)NCCCNC(=O)CCCCO[C@H]1O[C@H](CCP(=O)(O)O)[C@@H](O)[C@H](O)[C@@H]1O)NC(=O)CCc1cn(CCCCCC(=O)Oc2c(F)c(F)c(F)c(F)c2F)nn1] |
| 722 | 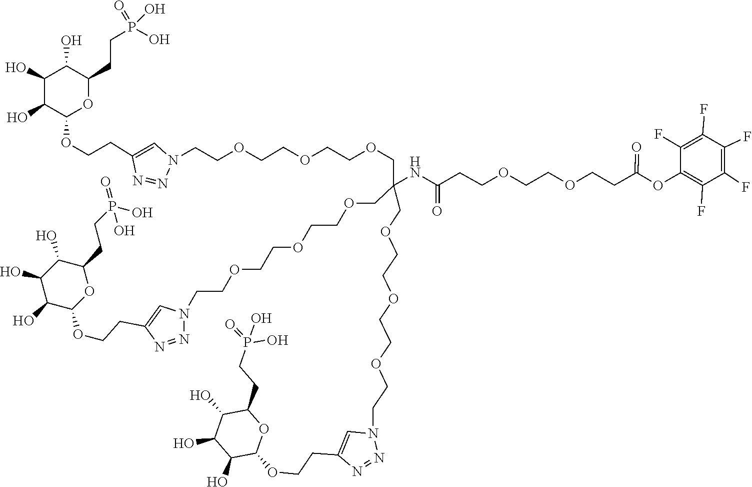 [SMILES: O=C(CCOCCOCCC(=O)Oc1c(F)c(F)c(F)c(F)c1F)NC(COCCOCCOCCn1cc(CCO[C@H]2O[C@H](CCP(=O)(O)O)[C@@H](O)[C@H](O)[C@@H]2O)nn1)(COCCOCCOCCn1cc(CCO[C@H]2O[C@H](CCP(=O)(O)O)[C@@H](O)[C@H](O)[C@@H]2O)nn1)COCCOCCOCCn1cc(CCO[C@H]2O[C@H](CCP(=O)(O)O)[C@@H](O)[C@H](O)[C@@H]2O)nn1] |

The structures of exemplary multivalent (n>1) compounds of this disclosure are shown in Table 12B which can be used to prepare conjugates of moieties of interest.

TABLE 12B
Exemplary multivalent compounds including amino acid linking moieties

| # | Structure |
|---|---|
| 751 | 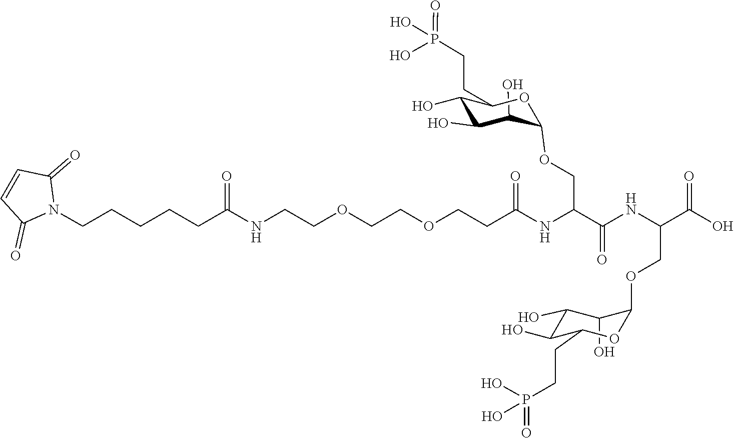 |
| 752 | 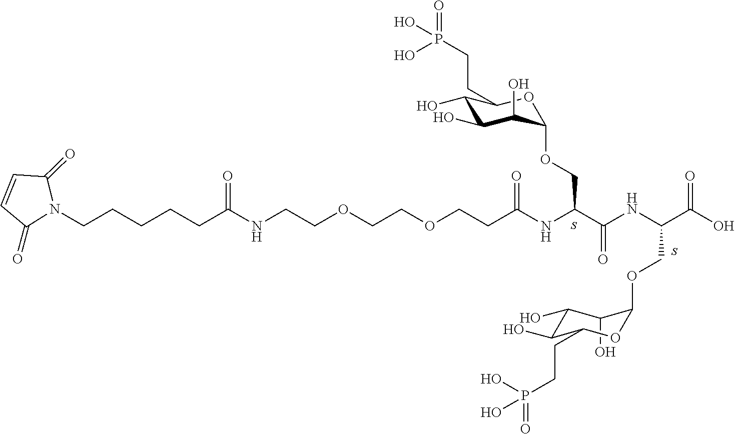 |
| 753 | 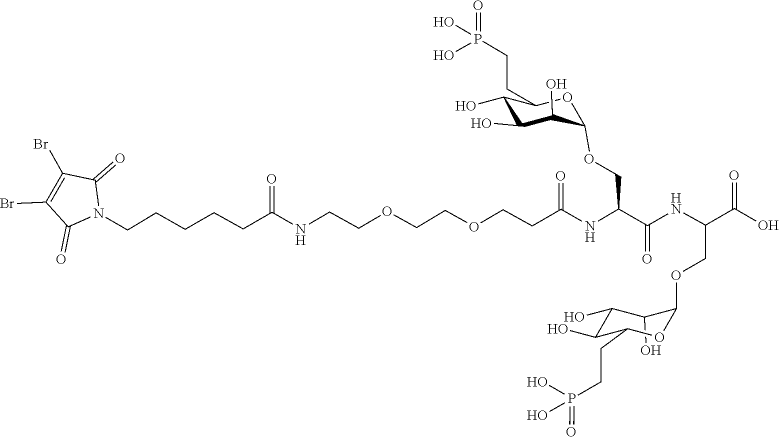 |
| 754 | 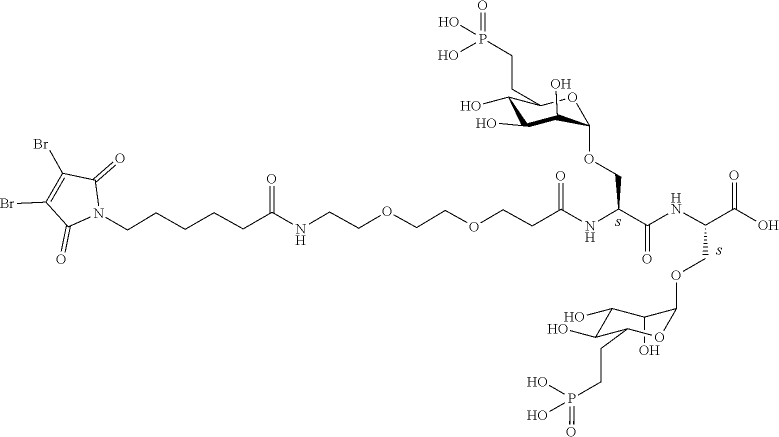 |
| 755 | 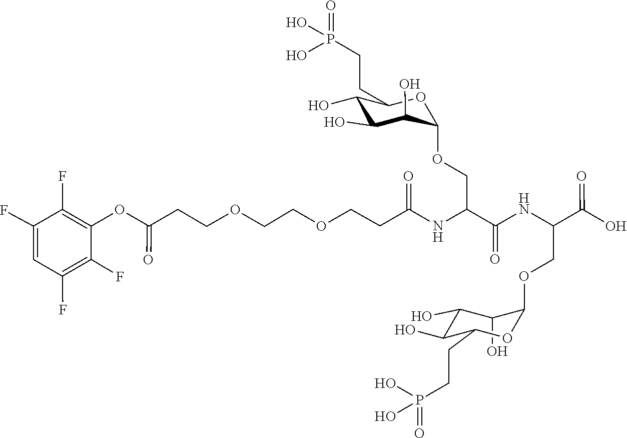 |
| 756 | 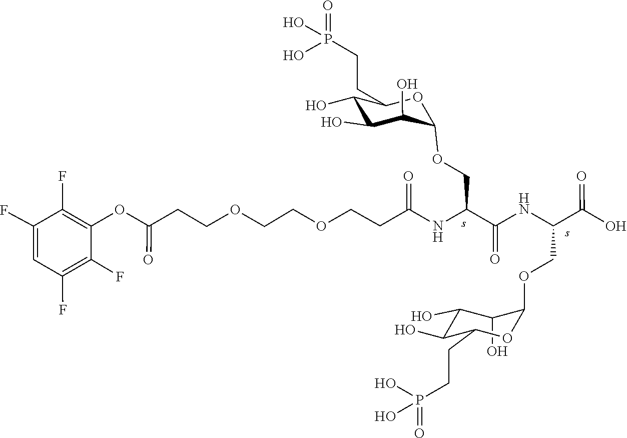 |
| 757 | 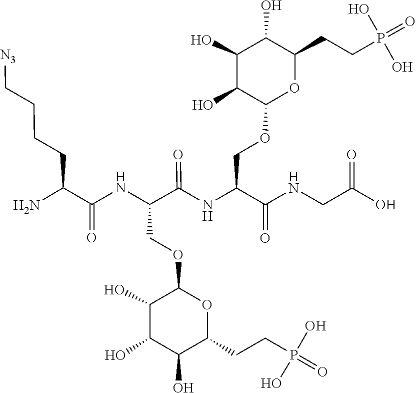 [SMILES: [N-]=[N+]=NCCCC[C@H](N)C(=O)N[C@@H](CO[C@H]1O[C@H](CCP(=O)(O)O)[C@@H](O)[C@H](O)[C@@H]1O)C(=O)N[C@@H](CO[C@H]1O[C@H](CCP(=O)(O)O)[C@@H](O)[C@H](O)[C@@H]1O)C(=O)NCC(=O)O] |
| 758 (I- 97) | 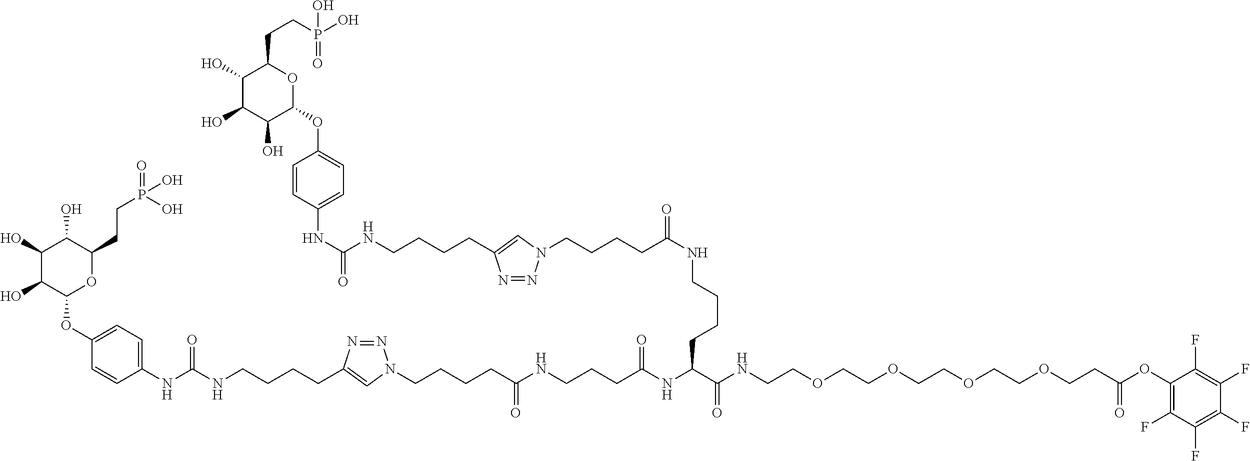 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 759 (I- 98) | 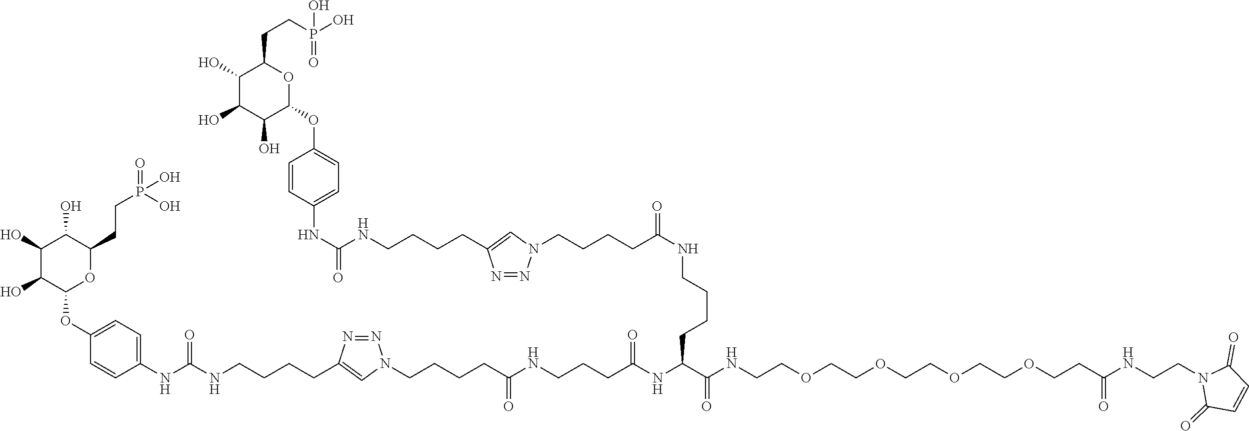 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| 760 (I- 99) | 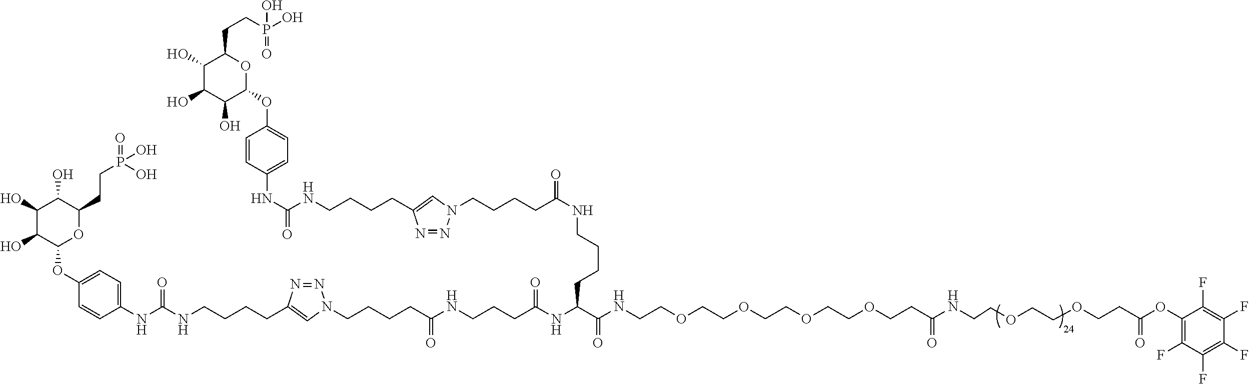 |
| 761 (I- 100) | 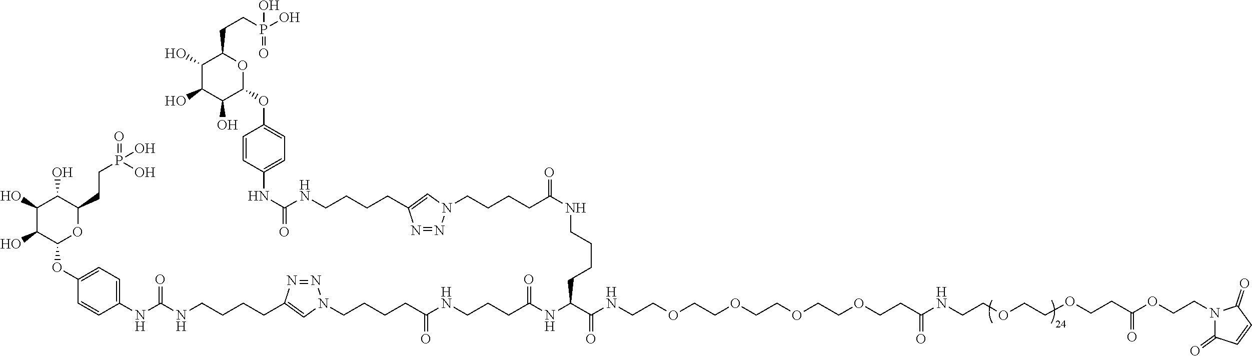 |
| 762 | 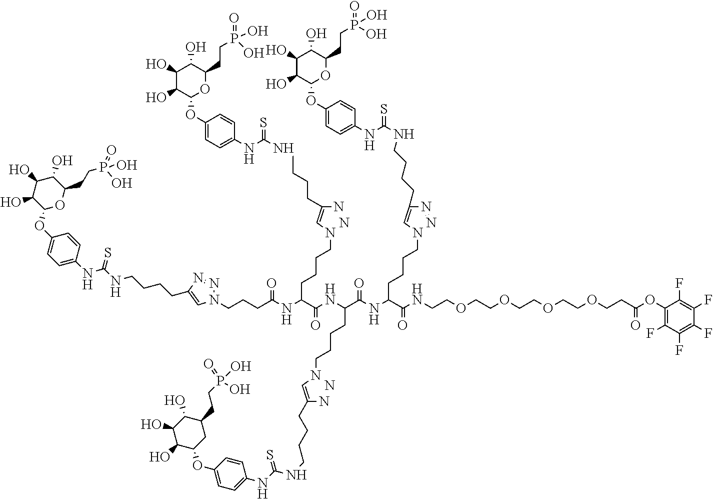 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3C[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 763 (I- 52) | 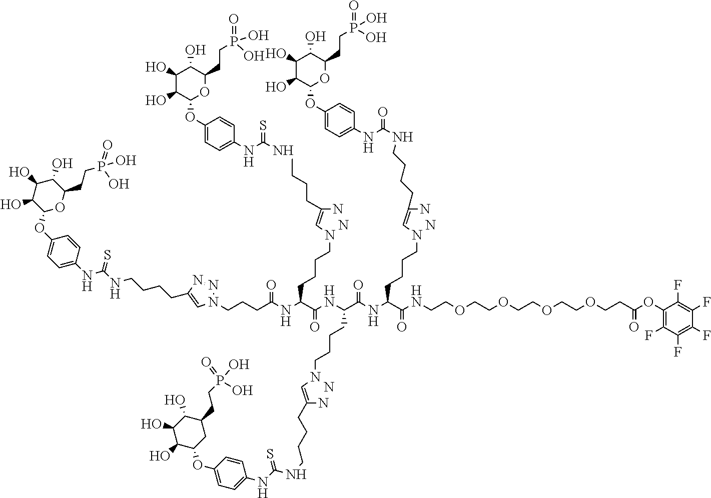 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3C[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 764 | 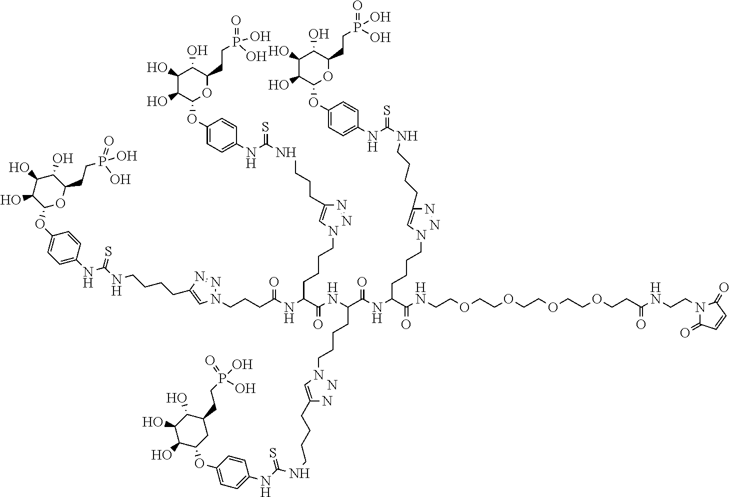 [SMILES: O=C(CCOCCOCCOCCOCCNC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3C[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 765 (I- 56) | 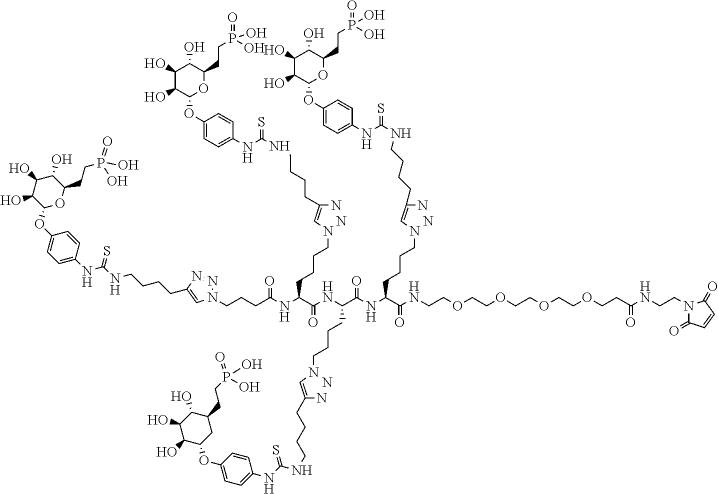 [SMILES: O=C(CCOCCOCCOCCOCCNC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3C[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 766 | 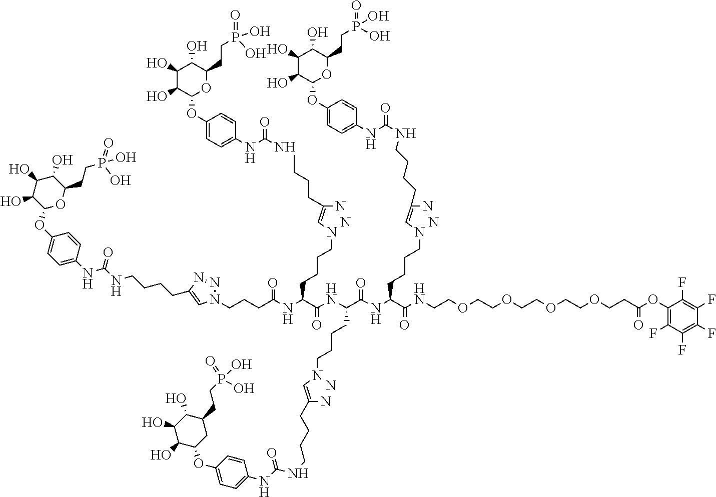 [SMILES: O=C(CCCn1cc(CCCCNC(=O)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3C[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 767 (I- 82) | 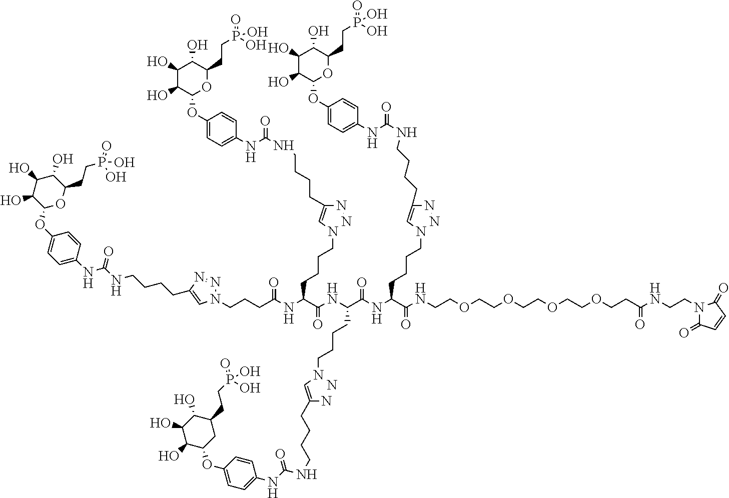 [SMILES: O=C(CCOCCOCCOCCOCCNC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3C[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)CCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 768 | 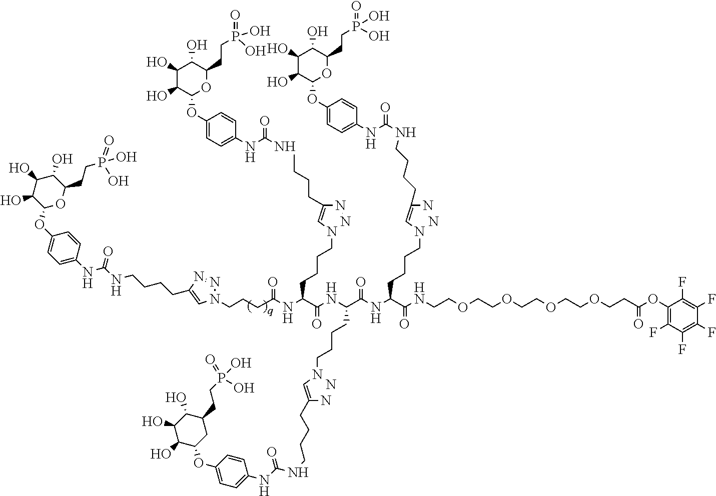 |
| | where q is 2 |
| 769 | 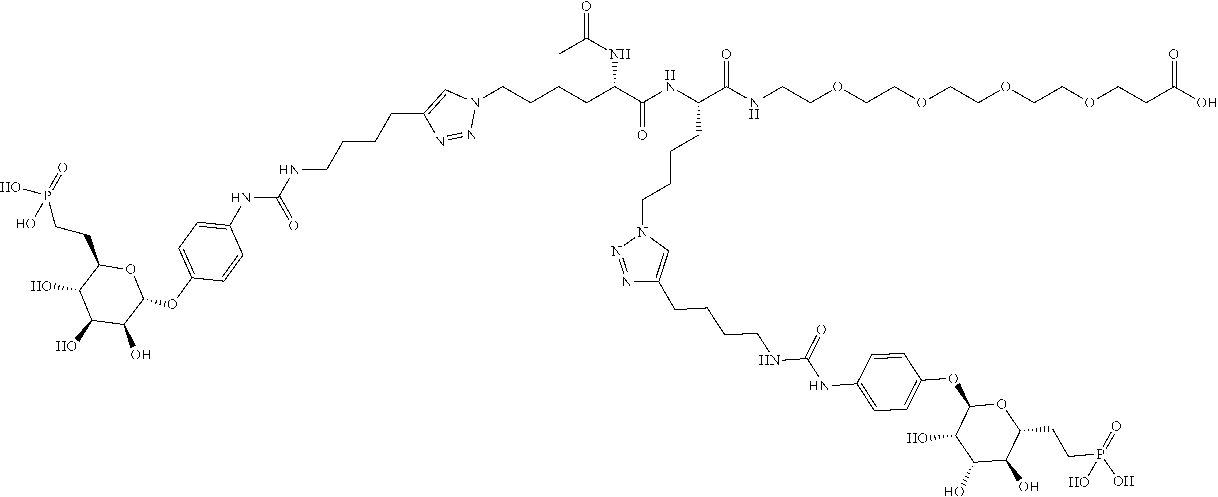 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)O] |
| 770 | 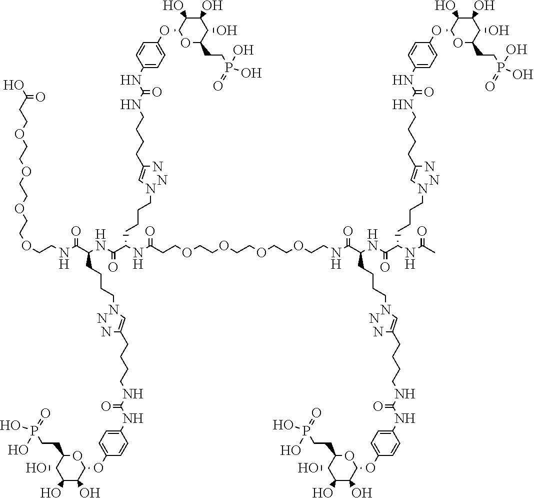 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)O] |
| 771 | 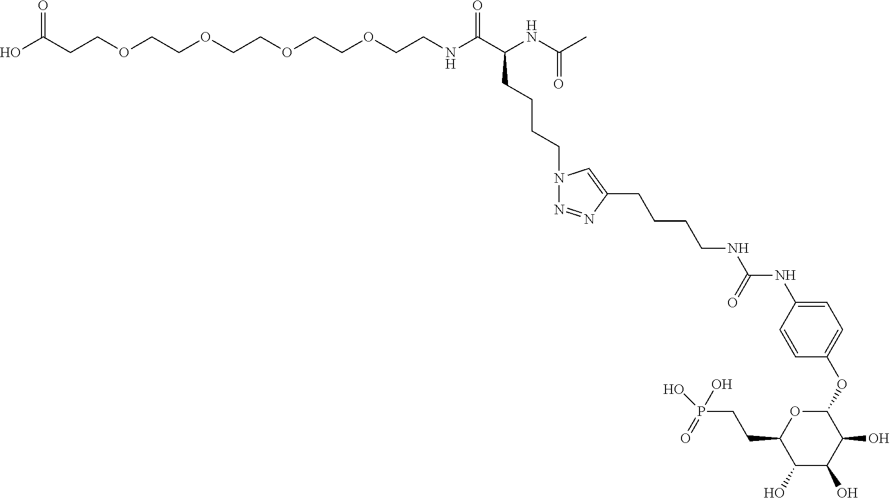 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)O] |
| 772 | 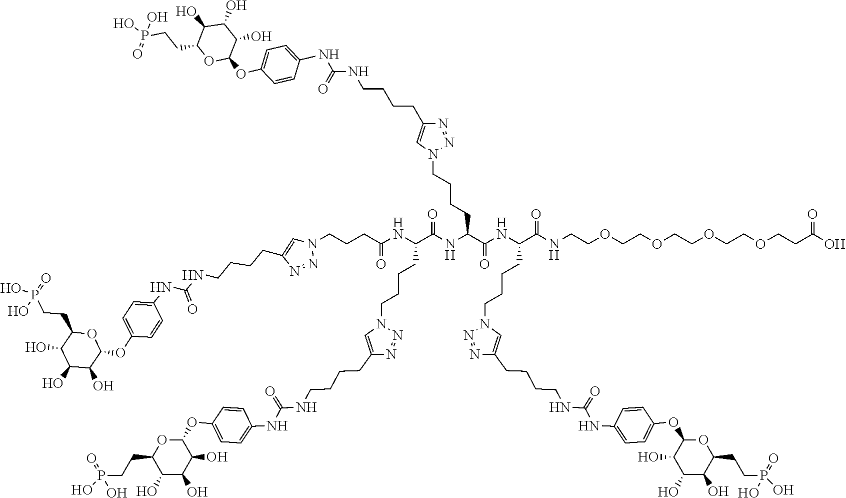 [SMILES: O=C(O)CCOCCOCCOCCOCCNC(=O)[C@H](CCCCn1cc(CCCCNC(=O)Nc2ccc(O[C@H]3O[C@@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)CCCn1cc(CCCCNC(=O)Nc2ccc(O[C@H]3O[C@H](CCP(=O)(O)O)[C@@H](O)[C@H](O)[C@@H]3O)cc2)nn1] |
| 773 | 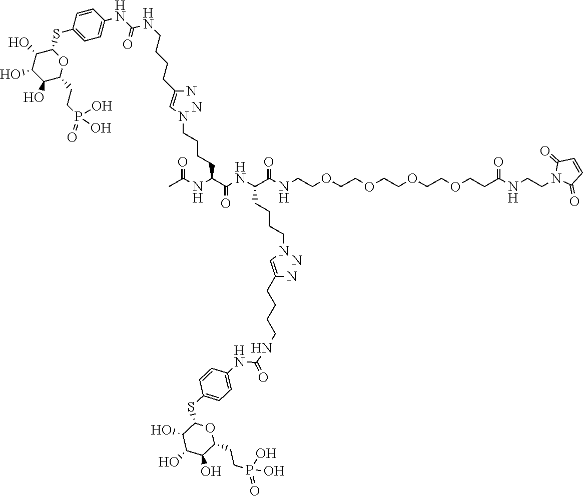 [SMILES: CC(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| 774 | 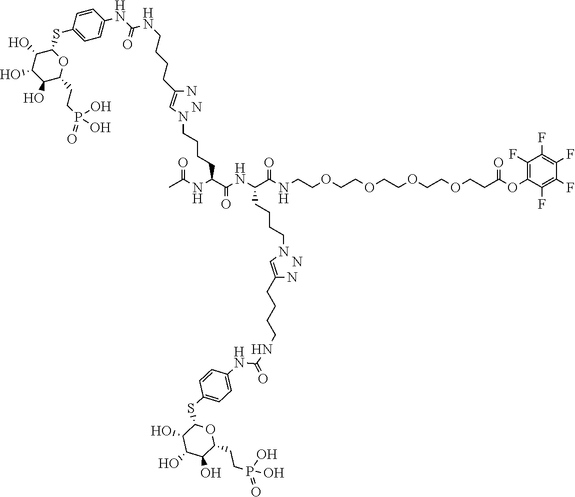 [SMILES: CC(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 775 | 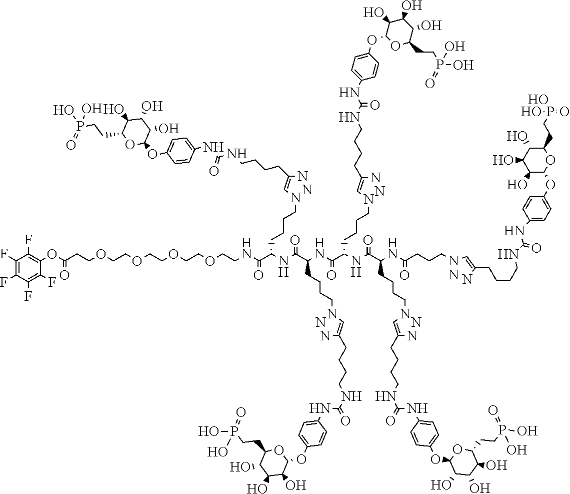 |
| 776 | 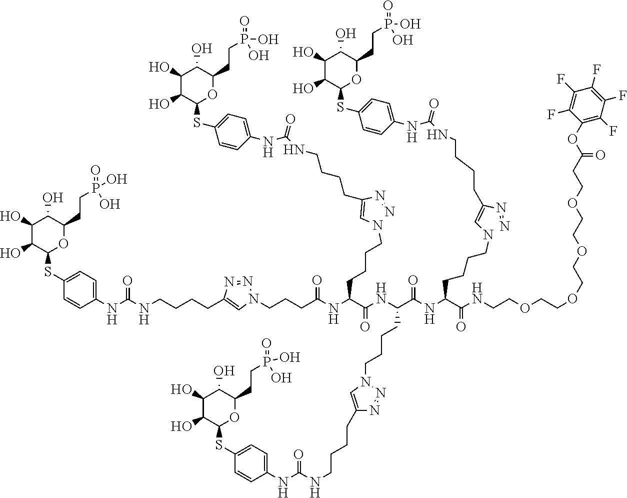 [SMILES: O=C(CCCn1cc(CCCCNC(=O)Nc2ccc(S[C@@H]3O[C@H](CCP(=O)(O)O)[C@@H](O)[C@H](O)[C@@H]3O)cc2)nn1)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 777 | 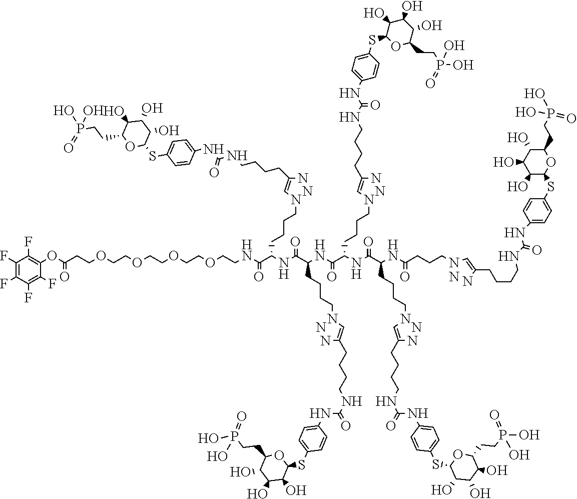 |
| 778-786 | 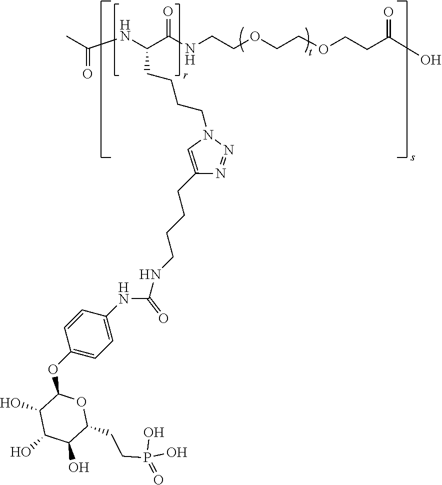 |
| | 778, where t = 3, s = 1, r = 1 |
| | 779, where t = 3, s = 1, r = 2 |
| | 780, where t = 3, s = 1, r = 3 |
| | 781, where t = 3, s = 2, r = 1 |
| | 782, where t = 3, s = 2, r = 2 |
| | 783, where t = 3, s = 2, r = 3 |
| | 784, where t = 3, s = 3, r = 1 |
| | 785, where t = 3, s = 3, r = 2 |
| | 786, where t = 3, s = 3, r = 3 |
| I-118F | 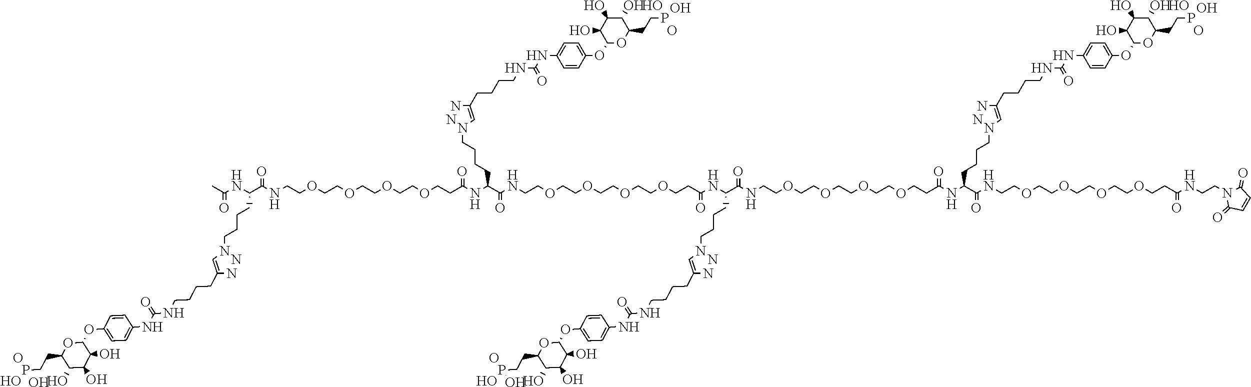 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| I-118G | 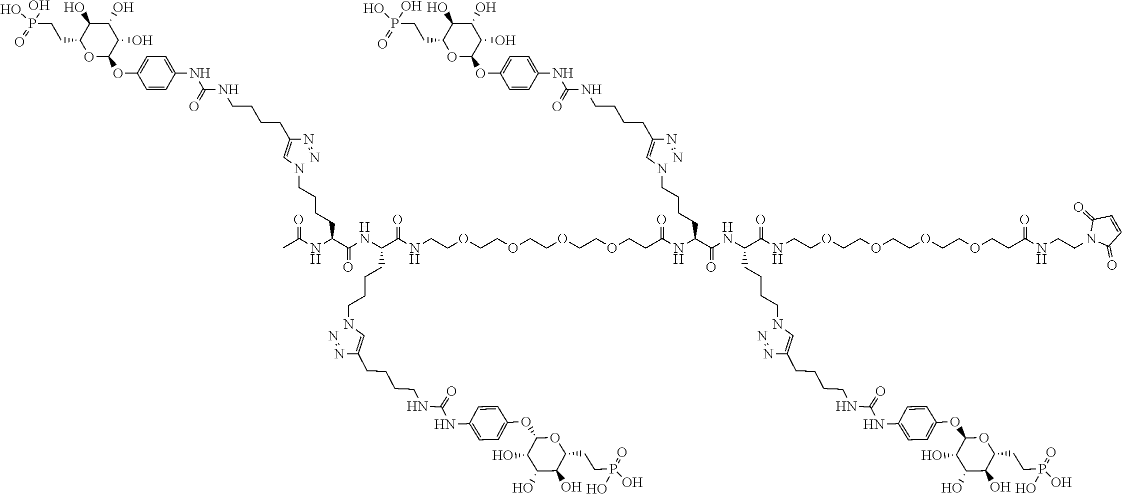 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| I-118H | 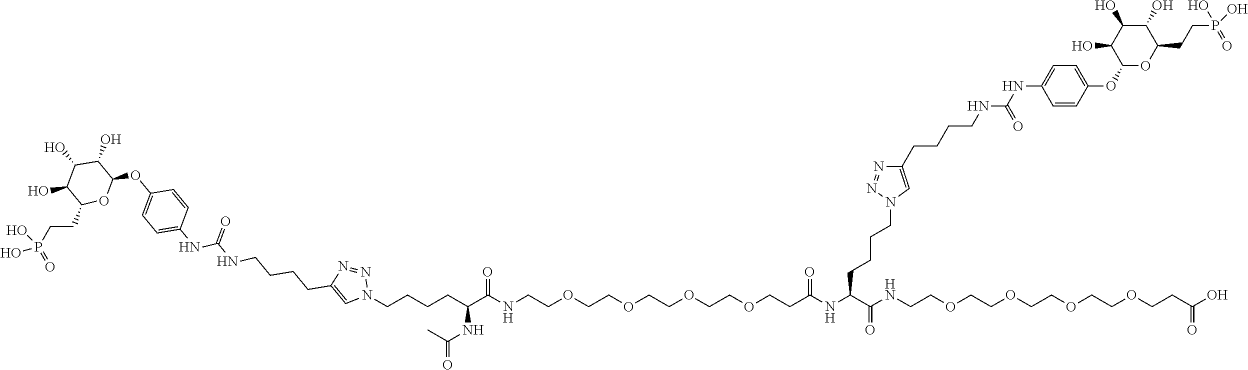 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)O] |

### 4.7. Additional Experimental Observations

Without being bound to any particular mechanism or theory, within a certain desired range, the binding affinity of a M6PR ligand may be inversely correlated to a longer half-life of the resulting compounds and conjugates, and selection of a desired binding affinity may be useful for tuning (e.g., modifying) the pharmacokinetic properties of the conjugates described herein. In certain embodiments, the compounds or conjugates having structures described herein can be selected to have a binding affinity to cell surface M6PR that provides for a combination of a desired pharmacokinetic property (e.g., sufficient half-life), while providing sufficiently robust uptake and/or degradation of the target.

### 4.8. Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier. Pharmaceutical compositions may be useful for the prevention, treatment, management or amelioration of a disease or disorder described herein or one or more symptoms thereof.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, PA Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### 4.9. Methods of Use

In one aspect, provided herein are methods of using the conjugates described herein to remove a polypeptide of interest (a target protein) from a cell's surface. In one aspect, provided herein are methods of using the conjugates described herein to remove a polypeptide of interest (a target protein) from the extracellular milieu. For example, in one embodiment, provided herein are methods of using the conjugates described herein to remove a polypeptide of interest (a target protein) from the surface of a cell by sequestering the target protein in the cell's lysosome. In another embodiment, provided herein are methods of using the conjugates described herein to remove a polypeptide of interest (a target protein) from the extracellular space (the extracellular milieu) of a cell by sequestering the target protein in the cell's lysosome. In another embodiment, provided herein are methods of using the conjugates described herein to remove a polypeptide of interest (a target protein) from the surface of a cell by sequestering the target protein in the cell's lysosome and degrading the target protein. In another embodiment, provided herein are methods of using the conjugates described herein to remove a polypeptide of interest (a target protein) from the extracellular space (the extracellular milieu) of a cell by sequestering the target protein in the cell's lysosome and degrading the target protein.

Removal of a target protein may refer to reduction, or depletion, of the target protein from the cell surface or from the extracellular space, or the extracellular milieu, that is, a reduction, or depletion, of the amount of the target protein on the cell surface or in the extracellular milieu. In some embodiments, the method is a method of reducing the amount or level of a target protein in a biological system or cellular sample.

In one aspect, provided herein are methods of using the conjugates described herein to sequester a polypeptide of interest (a target protein) in a cell's lysosome. In one aspect, provided herein are methods of using the conjugates described herein to sequester a polypeptide of interest (a target protein) in a cell's lysosome and to degrade the polypeptide of interest.

In one aspect, provided herein are methods of using the conjugates described herein to degrade a polypeptide of interest (a target protein).

In one aspect, provided herein are methods of depleting a polypeptide of interest (a target protein) described herein by degradation through a cell's lysosomal pathway.

In another aspect, provided herein are methods of depleting a polypeptide of interest (a target protein) described herein by administering to a subject in need thereof an effective amount of a conjugate or pharmaceutically acceptable salt described herein, or a pharmaceutical composition described herein. In certain embodiments, the subject is a mammal (e.g., human).

In certain embodiments, the target protein is a membrane bound protein. In certain embodiments, the target protein is a cell surface receptor. In certain embodiments, the target protein is an extracellular protein.

In certain embodiments, the target protein is a VEGF protein, an EGFR protein, a VEGFR protein, a PD-L1 protein, an FGFR2 protein or an FGFR3 protein.

In another aspect, provided herein are methods of treating a disease or disorder by administering to a subject, e.g., a human, in need thereof an effective amount of a conjugate or pharmaceutically acceptable salt described herein, or a pharmaceutical composition described herein.

The terms “administer,” “administration,” or “administering” refer to the act of injecting or otherwise physically delivering a substance (e.g., a conjugate or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

In certain embodiments, when the disorder or disease is cancer, “effective amount” or “therapeutically effective amount” mean that amount of a conjugate or pharmaceutical composition provided herein which, when administered to a human suffering from a cancer, is sufficient to effect treatment for the cancer. “Treating” or “treatment” of the cancer includes one or more of:
(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

In certain embodiments, the disease or disorder is treated by depletion of the target protein by degradation through the lysosomal pathway.

In certain embodiments, the disease or disorder is treated by depletion of certain proteins, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing.

In certain embodiments, the disease or disorder is a cancer.

In certain embodiments, the cancer is selected from the group consisting of bladder cancer, breast cancer, cervical cancer, cholangiocarcinoma, endometrial cancer, hepatocellular carcinoma, kidney cancer, melanoma, myeloid neoplasms, non-small cell lung cancer (NSCLC), Ewing's sarcoma, and Hodgkin's Lymphoma.

In certain embodiments, the cancer is a solid tumor.

In certain embodiments, the disease or disorder is an inflammatory or autoimmune disease.

In certain embodiments, the disease or disorder is an inflammatory disease.

In certain embodiments, the disease or disorder is an autoimmune disease.

### 4.10. Definitions

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a” “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. In certain embodiments, a polypeptide can occur as a single chain or as two or more associated chains, e.g., may be present as a multimer, e.g., dimer, a trimer. The terms also encompass an amino acid polymer that has been modified naturally or by intervention; for example, by disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein in their broadest sense and includes certain types of immunoglobulin molecules comprising one or more antigen-binding domains that specifically bind to an antigen or epitope.

In a certain embodiments, an isolated antibody (e.g., monoclonal antibody) described herein, or an antigen-binding fragment thereof, which specifically binds to a protein of interest, for example, EGFR, is conjugated to one or more lysosomal targeting moieties, for example, via a linker.

An “antigen” is a moiety or molecule that contains an epitope to which an antibody can specifically bind. As such, an antigen is also is specifically bound by an antibody. In a specific embodiment, the antigen, to which an antibody described herein binds, is a protein of interest, for example, EGFR (e.g., human EGFR), or a fragment thereof, or for example, an extracellular domain of EGFR (e.g., human EGFR).

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

An antibody specifically includes, but is not limited to, full length antibodies (e.g., intact immunoglobulins), antibody fragments, monoclonal antibodies, polyclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and D0bel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, conjugate function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. Antibody fragments suitable for use in the compounds of this disclosure include, for example, Fv fragments, Fab fragments, F(ab′)₂ fragments, Fab′ fragments, scFv (sFv) fragments, and scFv-Fc fragments.

“Polynucleotide” or “nucleic acid,” as used interchangeably herein, and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. The nucleic acid molecule may be an aptamer.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides, polypeptides and virus particles of interest are well-known in the art and include, for example, ion-exchange chromatography, affinity chromatography and sedimentation according to density.

The terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in of tumor burden).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O ¹⁸F, ³⁶Cl, ⁸²Br, ¹²³, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

A “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)n- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO2-alkyl, —SO2-aryl, —SO2-heteroaryl, and —NRaRb, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group refers to an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl”, unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” refer to di-radical alkenyl, alkynyl, aryl, aralkyl, and alkaryl groups, respectively.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocycloalkyl” refers to a cycloalkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N—O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The terms “heterocycle,” “heterocyclic” and “heterocyclyl” refer to a saturated or unsaturated group having a single ring or multiple condensed rings, including fused bridged and spiro ring systems, and having from 3 to 15 ring atoms, including 1 to 4 hetero atoms. These ring heteroatoms are selected from nitrogen, sulfur and oxygen, wherein, in fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C≡N), isocyanato (—O—N+≡C—), isothiocyanato (—S—C≡N), azido (—N═N+═N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO-NH—), imide (—CO—NH-CO—), epoxy (—O—), epithio (—S—), epidioxy (—O—O—), carbonyldioxy (—O—CO-O—), alkyldioxy (—O—(CH2)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N-OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻ M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O-M+, —OC(O)OR⁷⁰, —OC(S)OR⁷⁰, —NR⁷⁰C(O)R⁷⁰, —NR⁷⁰C(S)R⁷⁰, —NR⁷⁰CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸OR⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other atypical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

Definitions of other terms and concepts appear throughout the detailed description.

M6PR binding compounds and conjugates are described in International Application No. PCT/US2021/012846, filed Jan. 8, 2021, the disclosure of which is herein incorporated by reference in its entirety.

### 4.11. Additional Embodiments

Additional embodiments of the present disclosure are also described in the following clauses.

Clause 1. A cell surface mannose-6-phosphate receptor (M6PR) binding compound of formula:

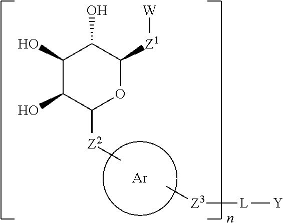

or a salt thereof, wherein:
each W is independently a hydrophilic head group;
each Z¹ is independently selected from optionally substituted (C₁-C₃)alkylene and optionally substituted ethenylene;
each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
each Ar is independently an optionally substituted aryl or heteroaryl linking moiety (e.g., monocyclic or bicyclic aryl or heteroaryl, optionally substituted);
each Z³ is independently a linking moiety;
n is 1 to 500;
   L is a linker; and
   Y is a moiety of interest;
wherein when m is 1 and Ar is phenyl, then: i) L comprises a backbone of at least 16 consecutive atoms; ii) Y is a biomolecule; and/or ii) Z³ is amide, sulfonamide, urea or thiourea.

Clause 2. The compound of clause 1, wherein each Ar is independently selected from optionally substituted phenyl, optionally substituted pyridyl, optionally substituted biphenyl, optionally substituted naphthalene, optionally substituted triazole and optionally substituted phenylene-triazole.

Clause 3. The compound of clause 2, wherein Ar is selected from optionally substituted 1,4-phenylene, optionally substituted 1,3-phenylene, or optionally substituted 2,5-pyridylene.

Clause 4. The compound of clause 3, wherein the compound is of one of formula:

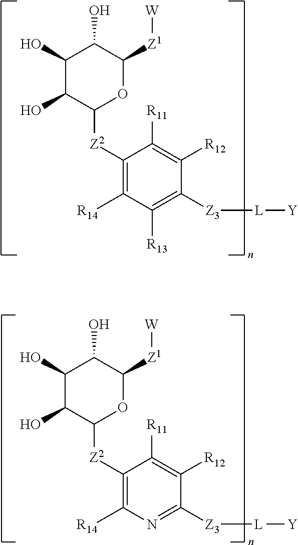

or a salt thereof, wherein:
each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each R²⁵ is independently selected from H, and optionally substituted (C₁-C₆)alkyl.

Clause 5. The compound of clause 1, wherein Ar is an optionally substituted fused bicyclic aryl or fused bicyclic heteroaryl.

Clause 6. The compound of clause 5, wherein Ar is optionally substituted naphthalene or an optionally substituted quinoline.

Clause 7. The compound of clause 6, wherein the compound is of one of formula:

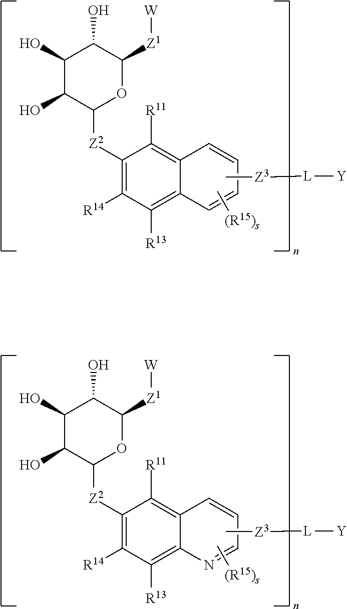

or a salt thereof, wherein:
each R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

Clause 8. The compound of clause 7, wherein the compound is of one of following formula:

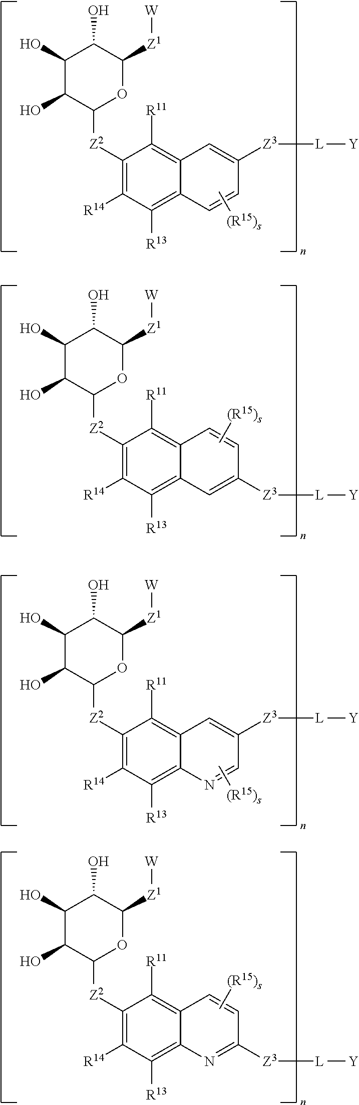

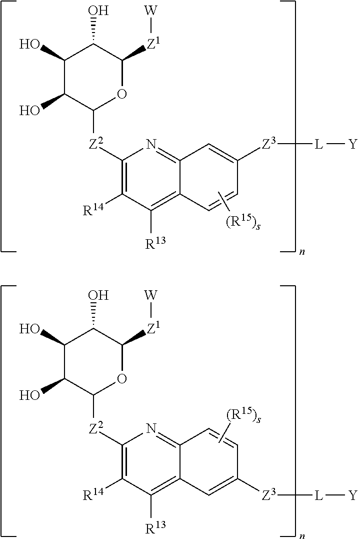

or a salt thereof.

Clause 9. The compound of clause 1, wherein Ar is optionally substituted bicyclic aryl or optionally substituted bicyclic heteroaryl and wherein the compound is of formula

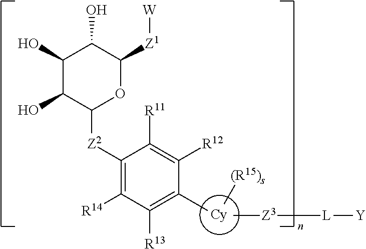

or a salt thereof, wherein:
each Cy is independently monocyclic aryl or monocyclic heteroaryl;
each R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²⁵ is independently selected from H, and optionally substituted (C₁-C₆)alkyl.

Clause 10. The compound of clause 9, wherein Ar is optionally substituted biphenyl, Cy is optionally substituted phenyl, and the compound is of formula:

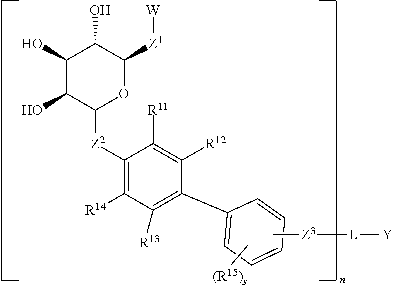

or a salt thereof.

Clause 11. The compound of clause 10, wherein the compound is of one of following formula:

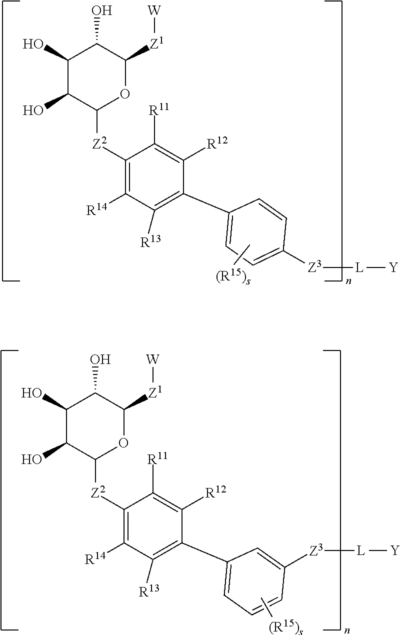

or a salt thereof.

Clause 12. The compound of any one of clauses 1 to 10, wherein Ar is substituted with at least one OH substituent.

Clause 13. The compound of any one of clauses 4, 6, 7, 9 and 10, wherein R¹¹ to R¹⁵ are each H.

Clause 14. The compound of any one of clauses 4, 6, 7, 9 and 10, wherein at least one of R¹¹ to R¹⁵ is OH (e.g., at least two are OH).

Clause 15. The compound of any one of clauses 1 to 14, wherein:
Z³ is selected from a covalent bond, —O—, —NR²³—, —NR²³CO—, —CONR²³—, —NR²³CO₂—OCONR²³, —NR²³C(═X¹)NR²³—, —CR²⁴═N—, —CR²⁴═N—X², —N(R²³)SO₂— and —SO₂N(R²³)—;
X¹ and X² are selected from O, S and NR²³; and
R²³ and R²⁴ are independently selected from H, C₍₁₋₃₎-alkyl (e.g., methyl) and substituted C₍₁₋₃₎-alkyl.

Clause 16. The compound of any one of clauses 1 to 15, wherein Z³ is

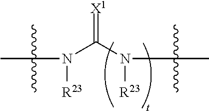

wherein: X¹ is O or S; t is 0 or 1; and each R²³ is independently selected from H, C₍₁₋₃₎-alkyl (e.g., methyl) and substituted C₍₁₋₃₎-alkyl.

Clause 17. The compound of clause 16, wherein Z³ is —NHC(═X¹)NH—, wherein X¹ is O or S.

Clause 18. The compound of any one of clauses 1 to 14, wherein Ar is triazole and the compound is of one of following formula:

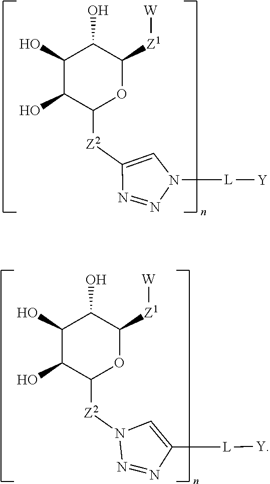

Clause 19. The compound of clause 18, wherein Z³ is optionally substituted triazole and the compound is of one of following formula

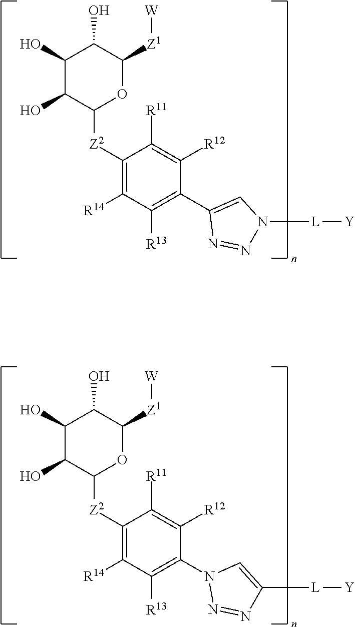

or a salt thereof,
wherein:
   each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
   each R²⁵ is independently selected from H, and optionally substituted (C₁-C₆)alkyl.

Clause 20. The compound of any one of clauses 1 to 19, wherein —Ar—Z³— is selected from:

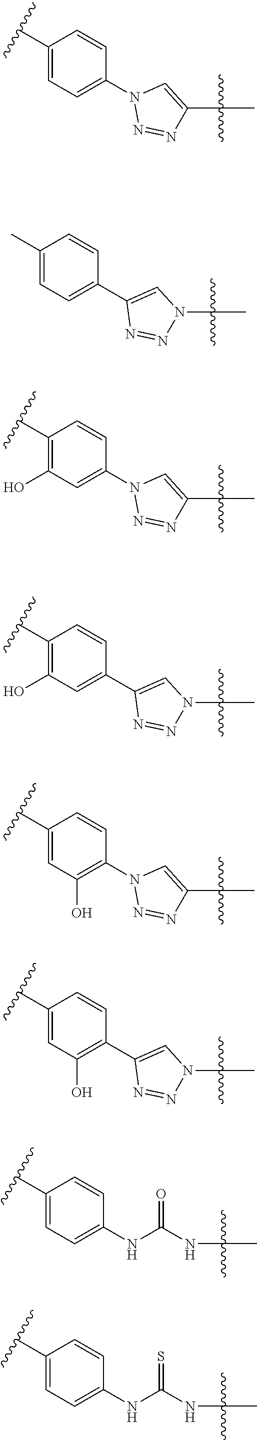

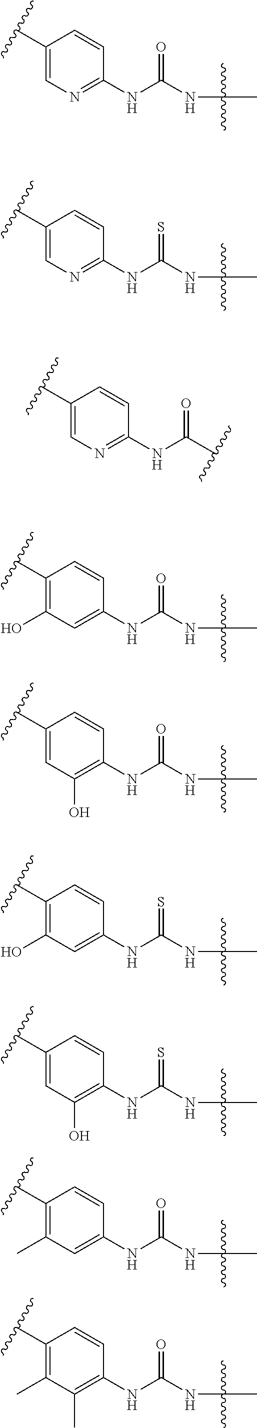

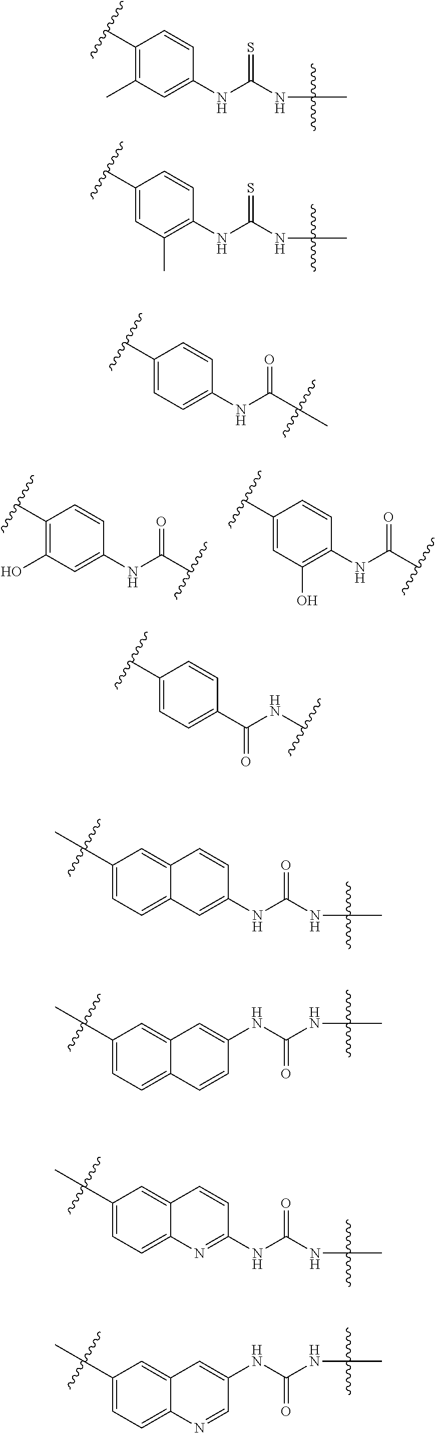

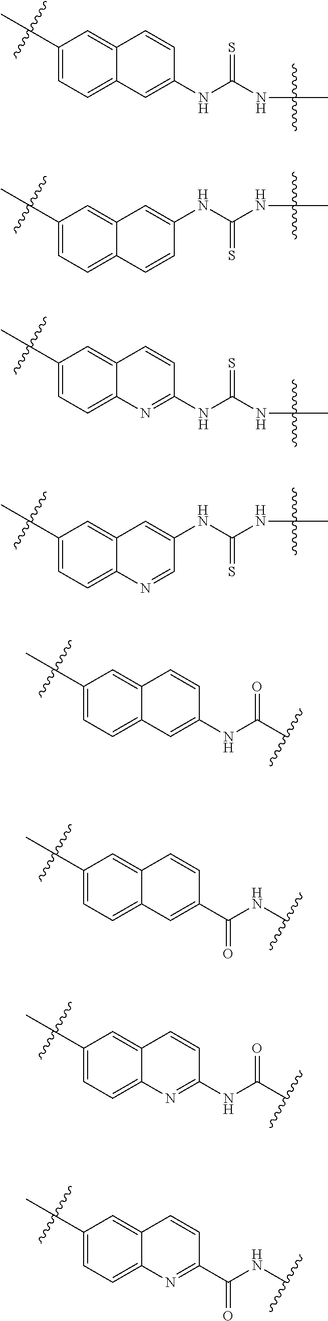

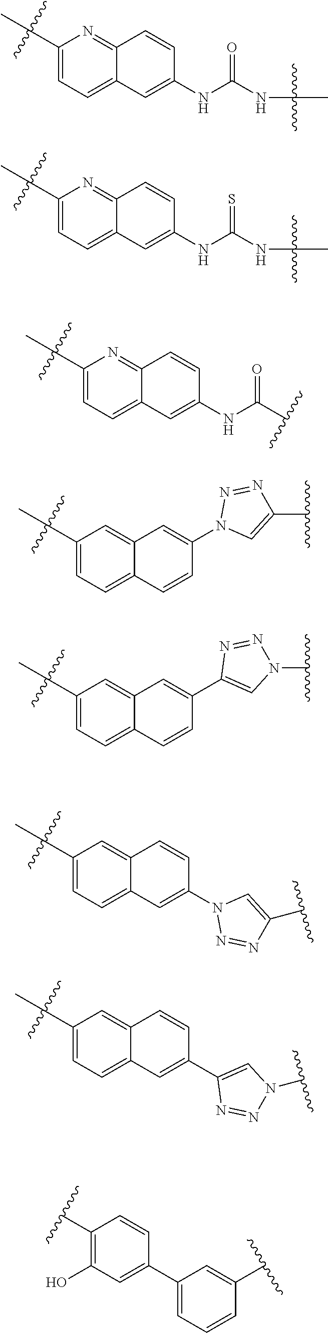

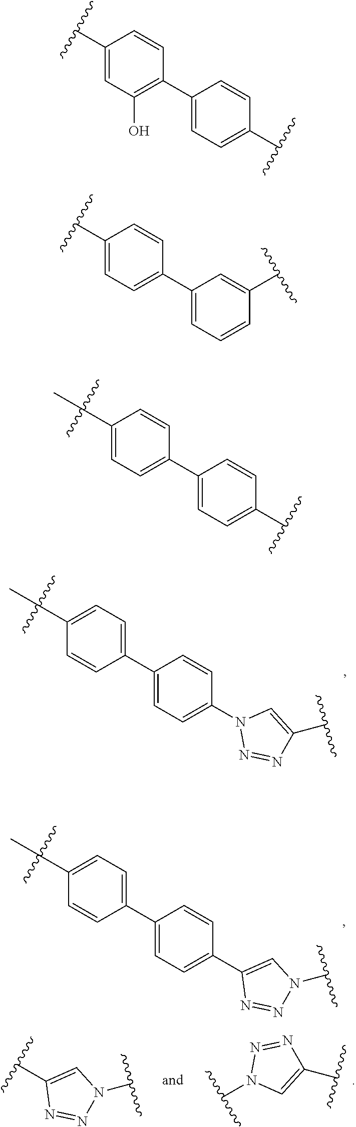

Clause 21. The compound of any one of clauses 1 to 20, wherein m is at least 2, and L is a branched linker that covalently links each Ar group to Y.

Clause 22. The compound of clause 21, wherein m is 2 to 20 (e.g., m is 2 to 6, such as 2 or 3).

Clause 23. The compound of clause 21, wherein: m is 20 to 500 (e.g., 20 to 400, 20 to 300, or 20 to 200, or 50 to 500, or 100 to 500); and L is an α-amino acid polymer (e.g., poly-L-lysine) wherein a multitude of —Ar—Z³-groups are covalently linked to the polymer backbone via sidechain groups (e.g., via conjugation to the sidechain amino groups of lysine residues).

Clause 24. The compound of any one of clauses 21 to 23, wherein m is at least 2 and each Z³ linking moiety is separated from every other Z³ linking moiety by a chain of at least 16 consecutive atoms via linker L (e.g., by a chain of at least 20, at least 25, or at least 30 consecutive atoms, and in some cases by a chain of up to 100 consecutive atoms).

Clause 25. The compound of any one of clauses 1 to 24, wherein the compound is of formula:

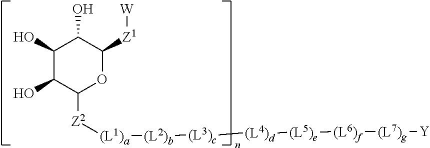

or a salt thereof, wherein:
n is 1 to 500 (e.g., n is 1 to 20, 1 to 10, 1 to 6 or 1 to 5);
each L¹ to L⁷ is independently a linking moiety that together provide a linear or branched linker between the n Z² groups and Y, and wherein -(L′)ₐ- comprises the linking moiety Ar that is optionally substituted aryl or heteroaryl group;
a is 1 or 2; and
b, c, d, e, f, and g are each independently 0, 1, or 2.

Clause 26. The compound of clause 25, wherein the linear or branched linker separates each Z² and Y by a chain of at least 16 consecutive atoms (e.g., at least 20 consecutive atoms, at least 30 consecutive atoms, or 16 to 100 consecutive atoms).

Clause 27. The compound of any one of clauses 25 to 26, wherein n is 1 to 20.

Clause 28. The compound of any one of clauses 25 to 27, wherein n is at least 2 (e.g., n is 2 or 3).

Clause 29. The compound of clause 28, wherein d is >0 and L⁴ is a branched linking moiety that is covalently linked to each L¹ linking moiety.

Clause 30. The compound of any one of clauses 25 to 29, wherein the compound is of formula

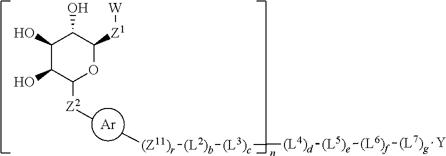

wherein:
Ar is an optionally substituted aryl or heteroaryl group (e.g., monocyclic or bicyclic or tricyclic aryl or heteroaryl group);
Z¹¹ is a linking moiety (e.g., covalent bond, heteroatom, group having a backbone of 1-3 atoms in length or triazole);
r is 0 or 1; and
n is 1 to 6.

Clause 31. The compound of clause 30, wherein Ar is selected from optionally substituted phenyl, optionally substituted pyridyl, optionally substituted biphenyl, optionally substituted naphthalene, optionally substituted quinoline, optionally substituted triazole, optionally substituted phenyl-triazole, optionally substituted biphenyl-triazole, and optionally substituted naphthalene-triazole.

Clause 32. The compound of clause 31, wherein Ar is optionally substituted 1,4-phenylene.

Clause 33. The compound of any one of clauses 30 to 32, wherein Ar substituted with at least one hydroxy.

Clause 34. The compound of any one of clauses 25 to 33, wherein L¹ or —Ar—(Z¹¹)ₜ— is selected from:

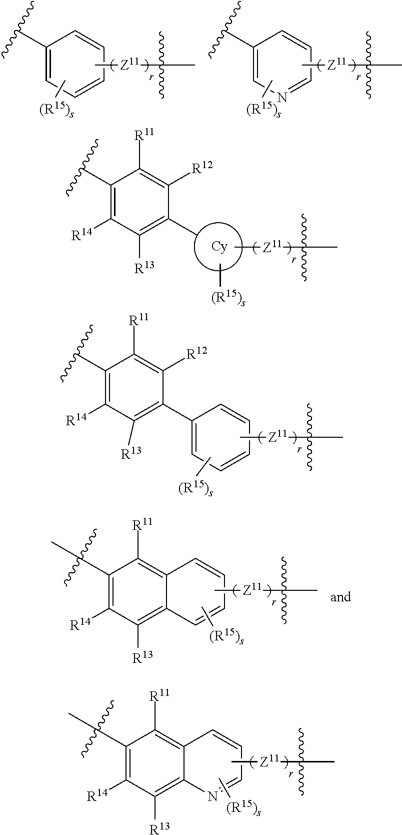

wherein:
Cy is monocyclic aryl or heteroaryl;
r is 0 or 1;
s is 0 to 4;
R¹¹ to R¹⁴ and each R¹⁵ are independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵, wherein each R²⁵ is independently selected from H, C₍₁₋₆₎-alkyl and substituted C₍₁₋₆₎-alkyl; and
Z¹¹ is selected from covalent bond, —O—, —NR²³—, —NR²³CO—, —CONR²³—, —NR²³CO₂—OCONR²³, —NR²³C(═X¹)NR²³—, —CR²⁴═N—, —CR²⁴═N—X²— and optionally substituted triazole, where X¹ and X² are selected from O, S and NR²³, wherein R²³ and R²⁴ are independently selected from H, C₍₁₋₃₎-alkyl (e.g., methyl) and substituted C₍₁₋₃₎-alkyl.

Clause 35. The compound of clause 34, wherein L¹ is

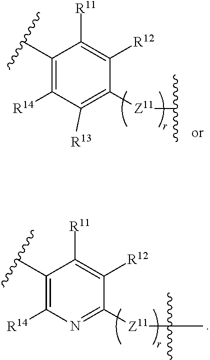

Clause 36. The compound of clause 34, wherein L¹ is

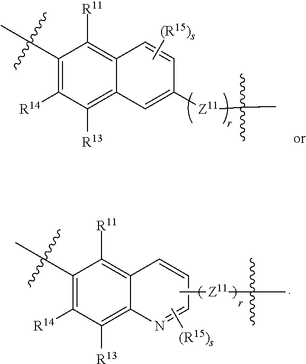

Clause 37. The compound of clause 34, wherein L¹ is selected from:

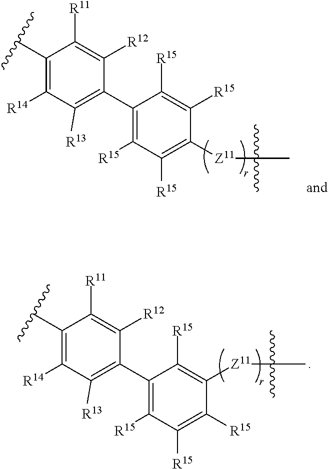

Clause 38. The compound of any one of clauses 34 to 37, wherein r is 0.

Clause 39. The compound of any one of clauses 34 to 37, wherein r is 1 and Z¹¹ is selected from —O—, —NR²³—, —NR²³CO—, CONR²³—, —NR²³CO₂—, —OCONR²³—, —NR²³C(═X¹)NR²³—, —CR²⁴═N—, and —CR²⁴═N—X²—, wherein X¹ and X² are selected from O, S and NR²³, and each R²³ and R²⁴ is independently selected from H, C₍₁₋₃₎-alkyl (e.g., methyl) and substituted C₍₁₋₃₎-alkyl.

Clause 40. The compound of any one of clauses 34 to 37, wherein r is 1 and Z¹¹ is

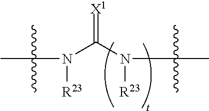

wherein: X¹ is O or S; t is 0 or 1; and each R²³ is independently selected from H, C₍₁₋₃₎-alkyl (e.g., methyl) and substituted C₍₁₋₃₎-alkyl.

Clause 41. The compound of clause 40, wherein Z¹¹ is —NHC(═X¹)NH—, wherein X¹ is O or S.

Clause 42. The compound of any one of clauses 34 to 37, wherein r is 1 and Z¹¹ is triazole.

Clause 43. The compound of any one of clauses 1 to 42, wherein Y is selected from small molecule, dye, fluorophore, monosaccharide, disaccharide, trisaccharide, and chemoselective ligation group or precursor thereof.

Clause 44. The compound of any one of clauses 1 to 42, wherein Y is a biomolecule.

Clause 45. The compound of clause 44, wherein the biomolecule is selected from peptide, protein, polynucleotide, polysaccharide, glycoprotein, lipid, enzyme, antibody, and antibody fragment.

Clause 46. The compound of any one of clauses 1 to 45, wherein Y is a moiety that specifically binds a target protein.

Clause 47. The compound of clause 46, wherein the target protein is a membrane bound protein.

Clause 48. The compound of clause 46, wherein the target protein is an extracellular protein.

Clause 49. The compound of any one of clauses 46 to 49, wherein Y is selected from antibody, antibody fragment (e.g., antigen-binding fragment of an antibody), chimeric fusion protein, an engineered protein domain, D-protein binder of target protein, aptamer, peptide, enzyme substrate and small molecule inhibitor or ligand.

Clause 50. The compound of clause 49, wherein Y is antibody or antibody fragment that specifically binds the target protein and the compound is of formula:

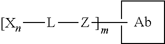

or a pharmaceutically acceptable salt thereof, wherein:
n is 1 to 20;
m is an average loading of 1 to 80;
Ab is the antibody or antibody fragment that specifically binds the target protein; and
Z is a residual moiety resulting from the covalent linkage of a chemoselective ligation group to a compatible group of Ab.

Clause 51. The compound of clause 49, wherein Y is a small molecule inhibitor or ligand of the target protein.

Clause 52. The compound of any one of clauses 1 to 51, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

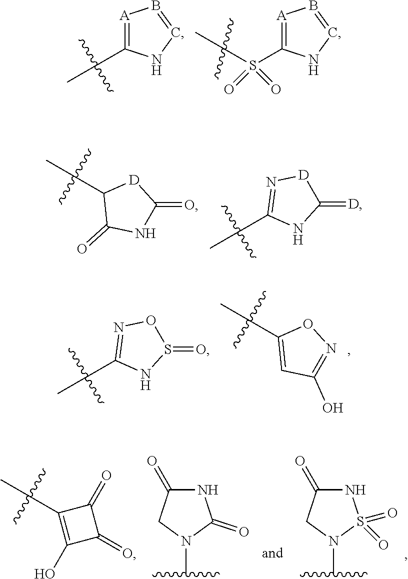

or a salt thereof,
wherein: R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃); A, B, and C are each independently CH or N; and D is each independently O or S.

Clause 53. The compound of clause 52, wherein W is selected from —P═O(OH)₂, —SO₃H, —COOH and —CH(COOH)₂, or a salt thereof.

Clause 54. The compound of any one of clauses 1 to 53, wherein: Z¹ is —(CH₂)ⱼ— or —(C(R²²)₂)ⱼ—, wherein each R²² is independently selected from H, halogen (e.g., F) and optionally substituted (C₁-C₆)alkyl; and j is 1 to 3.

Clause 55. The compound of any one of clauses 1 to 53, wherein Z¹ is —CH═CH—.

Clause 56. The compound of any one of clauses 1 to 55, wherein Z² is O or S.

Clause 57. The compound of any one of clauses 1 to 55, wherein Z² is —NR²¹—.

Clause 58. The compound of any one of clauses 1 to 55, wherein Z² is —C(R²²)₂—, wherein each R²² is independently selected from H, halogen (e.g., F) and optionally substituted (C₁-C₆)alkyl.

Clause 59. The compound of any one of clauses 1 to 53, wherein: Z¹ is selected from —(CH₂)ⱼ—, substituted (C₁-C₃)alkylene and —CH═CH—; j is 1 to 3; and Z² is selected from 0 and CH₂.

Clause 60. The compound of clause 60, wherein: Z¹ is —(CH₂)₂—, —CH₂—CF₂— or —CH2—CHF—; and Z² is O.

Clause 61. The compound of clause 60, wherein: Z¹ is —(CH₂)₂—, —CH₂—CF₂— or —CH2—CHF—; and Z² is CH₂.

Clause 62. The compound of clause 60, wherein: Z¹ is —CH═CH—; and Z² is O.

Clause 63. The compound of clause 60, wherein: Z¹ is —CH═CH—; and Z² is CH₂.

Clause 64. The compound of any one of clauses 1 to 63, wherein X is selected from:

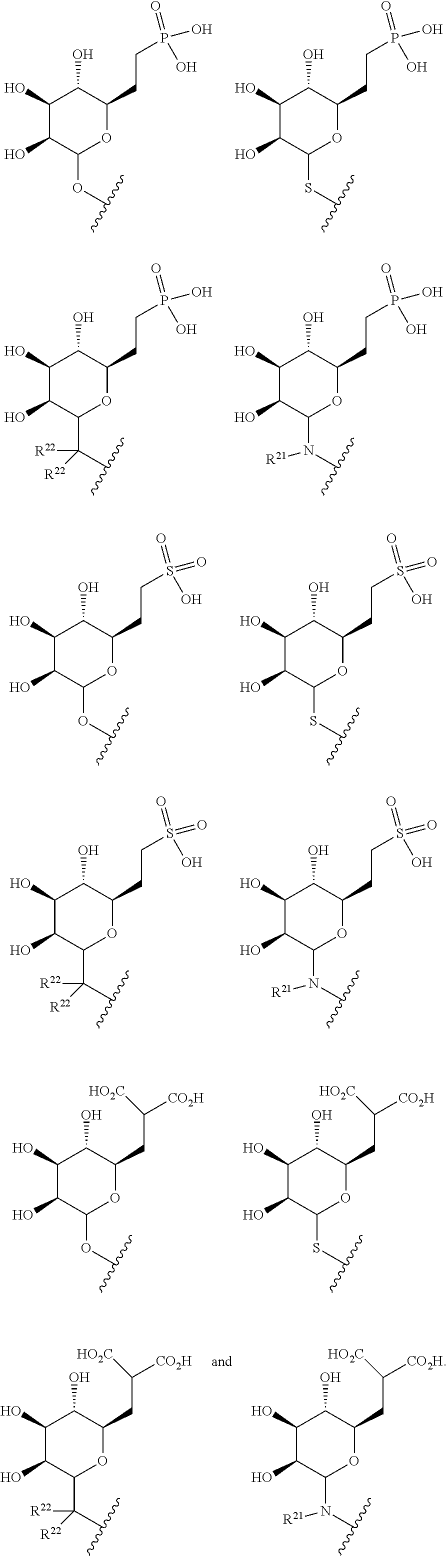

Clause 65. The compound of any one of clauses 25 to 64, wherein n is 1 to 6 (e.g., n is 1 to 5, or 2 to 6, or 1, 2 or 3), and wherein:
when d is 0, n is 1;
when d is 1, n is 1 to 3; and
when d is 2, n is 1 to 6.

Clause 66. The compound of any one of clauses 25 to 65, wherein: each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and
each L³ is independently selected from:

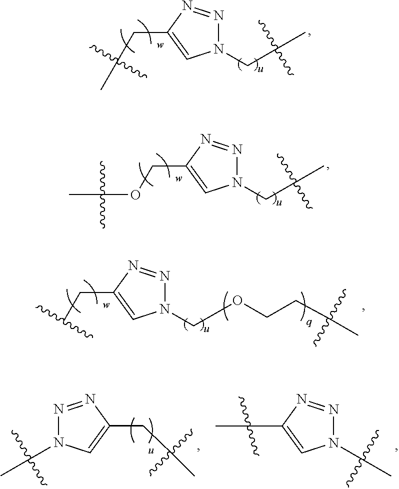

and —(OCH₂CH₂)_{q}—, wherein q is 1 to 10, u is 0 to 10, and w is 1 to 10.

Clause 67. The compound of any one of clauses 25 to 66, wherein when n is 2 or more, at least one L⁴ is present and is a branched linking moiety.

Clause 68. The compound of any one of clauses 25 to 67, wherein each L⁴ is independently selected from:

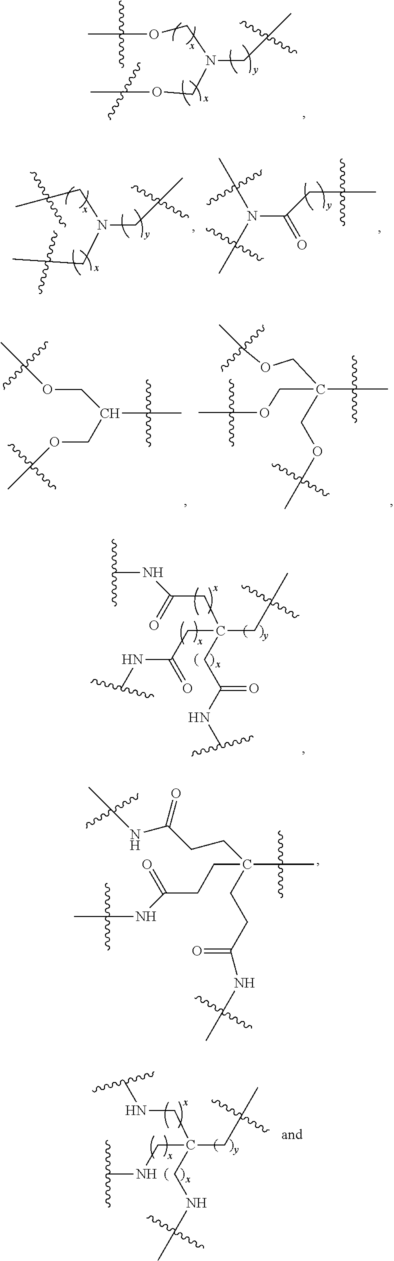

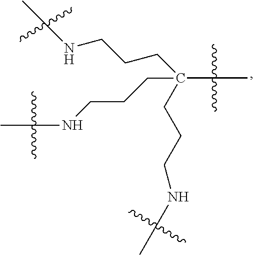

wherein each x and y are each independently 1 to 10.

Clause 69. The compound of any one of clauses 25 to 68, wherein: each L⁵ is independently —NHCO—C₁₋₆-alkylene-, —CONH—C₁₋₆-alkylene-, —C₁₋₆-alkylene-,

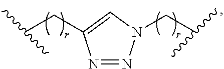

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and
r, s, and t are each independently 1 to 20.

Clause 70. The compound of any one of clauses 25 to 69, wherein a is 1.

Clause 71. The compound of any one of clauses 25 to 70, wherein at least one of b, c, e, f, and g is not 0.

Clause 72. The compound of any one of clauses 25 to 71, wherein at least one of b or c is not 0 and at least one of e, f, and g is not 0.

Clause 73. The compound of any one of clauses 25 to 72, wherein a, b, and c are each independently 1 or 2.

Clause 74. The compound of any one of clauses 1 to 73, wherein the linker L is selected from any one of the structures of Tables 2-3.

Clause 75. The compound of any one of clauses 1 to 74, wherein the compound is selected from the compounds of Tables 5-9.

Clause 76. A cell surface receptor binding conjugate of formula (I):

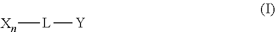

or a salt thereof, wherein:
X is a moiety that binds to a cell surface mannose-6-phosphate receptor (M6PR);
n is 1 to 500 (e.g., n is 1 to 20, 1 to 10, 1 to 6 or 1 to 5); and
L is a linker;
Y is a biomolecule that specifically binds a target protein.

Clause 77. The conjugate of clause 76, wherein the conjugate is of formula:

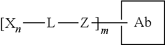

or a pharmaceutically acceptable salt thereof, wherein:
n is 1 to 20;
m is an average loading of 1 to 80;
Ab is an antibody or antibody fragment that specifically binds the target protein; and
Z is a residual moiety resulting from the covalent linkage of a chemoselective ligation group to a compatible group of Ab.

Clause 78. The conjugate of clause 76 or 77, wherein n is 1 to 6.

Clause 79. The conjugate of clause 76 or 77, wherein n is 2 or less.

Clause 80. The conjugate of clause 79, wherein n is 1.

Clause 81. The conjugate of clause 76 or 77, wherein n is at least 2.

Clause 82. The conjugate of clause 81, wherein n is 2.

Clause 83. The conjugate of clause 81, wherein n is 3.

Clause 84. The conjugate of clause 81, wherein n is 4.

Clause 85. The conjugate of any one of clauses 76 to 84, wherein m is 1 to 20.

Clause 86. The conjugate of any one of clauses 76 to 84, wherein m is 1 to 12.

Clause 87. The conjugate of any one of clauses 76 to 86, wherein m is at least about 2.

Clause 88. The conjugate of any one of clauses 76 to 86, wherein m is at least about 3.

Clause 89. The conjugate of any one of clauses 76 to 86, wherein m is at least about 4.

Clause 90. The conjugate of any one of clauses 77 to 89, wherein Z is a residual moiety resulting from the covalent linkage of a thiol-reactive chemoselective ligation group to one or more cysteine residue(s) of Ab.

Clause 91. The conjugate of any one of clauses 76 to 89, wherein Z is a residual moiety resulting from the covalent linkage of an amine-reactive chemoselective ligation group to one or more lysine residue(s) of Ab.

Clause 92. The conjugate of any one of clauses 76 to 91, wherein X is a moiety that binds M6PR and is of the formula:

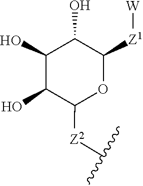

or a salt thereof, wherein:
each W is independently a hydrophilic head group;
each Z¹ is independently selected from optionally substituted (C₁-C₃)alkylene and optionally substituted ethenylene; and
each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

Clause 93. The conjugate of clause 92, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

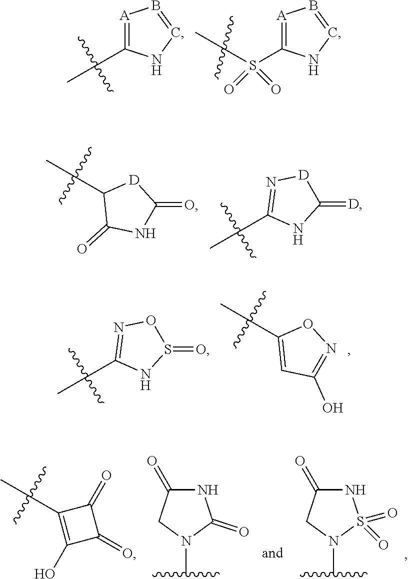

or a salt thereof,
wherein: R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃); A, B, and C are each independently CH or N; and D is each independently O or S.

Clause 94. The conjugate of clause 93, wherein W is selected from —P═O(OH)₂, —SO₃H, —CO₂H and —CH(CO₂H)₂, or a salt thereof.

Clause 95. The conjugate of any one of clauses 92 to 94, wherein Z¹ is —(CH₂)ⱼ— and j is 1 to 3.

Clause 96. The conjugate of any one of clauses 92 to 95, wherein Z¹ is —CH═CH—.

Clause 97. The conjugate of any one of clauses 92 to 96, wherein Z² is O or S.

Clause 98. The conjugate of any one of clauses 92 to 96, wherein Z² is —NR²¹—.

Clause 99. The conjugate of any one of clauses 92 to 96, wherein Z² is —C(R²²)₂—.

Clause 100. The conjugate of any one of clauses 92 to 94, wherein: Z¹ is selected from —(CH₂)ⱼ—, substituted (C₁-C₃)alkylene and —CH═CH—; j is 1 to 3; and Z² is selected from 0 and CH₂.

Clause 101. The conjugate of clause 100, wherein: Z¹ is —(CH₂)₂—, —CH₂—CF₂— or —CH₂—CHF—; and Z² is O.

Clause 102. The conjugate of clause 100, wherein: Z¹ is —(CH₂)₂—, —CH₂—CF₂— or —CH₂—CHF—; and Z² is CH₂.

Clause 103. The conjugate of clause 100, wherein: Z¹ is —CH═CH—; and Z² is O.

Clause 104. The conjugate of clause 100, wherein: Z¹ is —CH═CH—; and Z² is CH₂.

Clause 105. The conjugate of any one of clauses 92 to 104, wherein X is selected from:

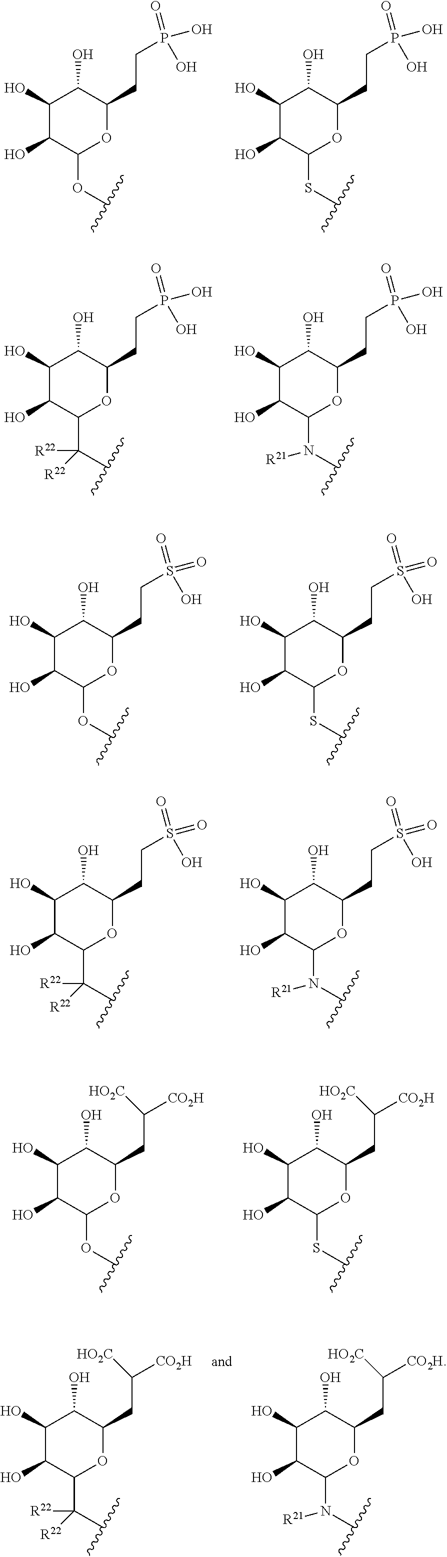

Clause 106. The conjugate of clauses 76 to 105, wherein the linker L is of formula (IIa):

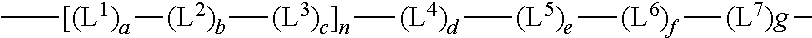

wherein
each L¹ to L⁷ is independently a linking moiety and together provide a linear or branched linker between X and Y;
a is 1 or 2;
b, c, d, e, f, and g are each independently 0, 1, or 2;
n is 1 to 6 (e.g., n is 1 to 5, or 2 to 6, or 1, 2 or 3).

Clause 107. The conjugate of clause 108, wherein:
when d is 0, n is 1;
when d is 1, n is 1 to 3; and
when d is 2, n is 1 to 6.

Clause 111. The conjugate of clause 109 or 110, wherein -(L′)ₐ- comprises an optionally substituted aryl or heteroaryl linking moiety.

Clause 112. The conjugate of clause 111, wherein each L¹ is independently selected from

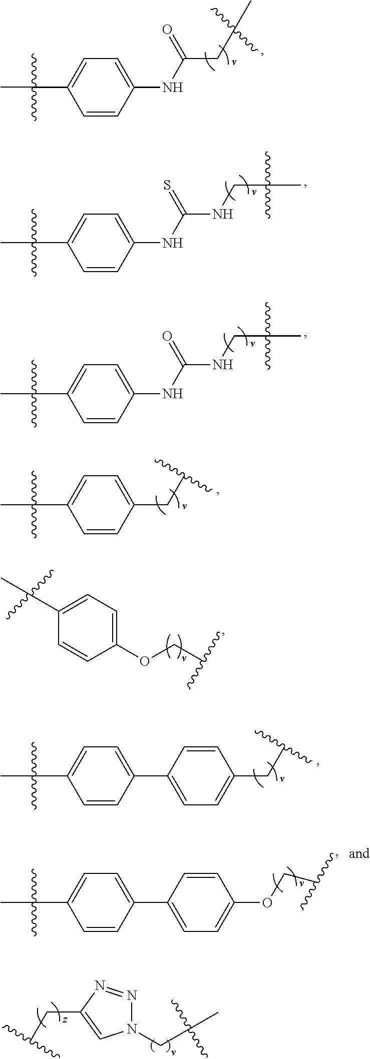

wherein v is 0 to 10 and z is 0 to 10.

Clause 113. The conjugate of any one of clauses 109 to 112, wherein: each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and
each L³ is independently selected from:

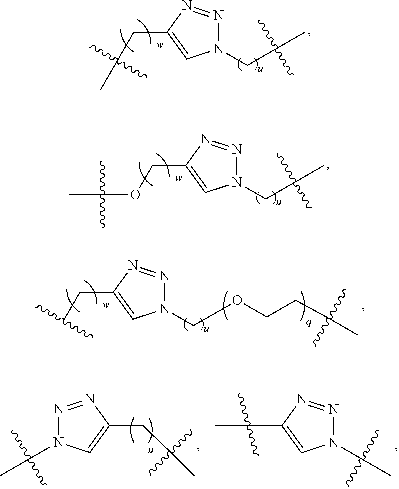

and —(OCH₂CH₂)_{q}—, wherein q is 1 to 10, u is 0 to 10, and w is 1 to 10.

Clause 114. The conjugate of any one of clauses 109 to 113, wherein when n is 2 or more, at least one L⁴ is present and is a branched linking moiety.

Clause 115. The conjugate of any one of clauses 109 to 114, wherein each L⁴ is independently selected from:

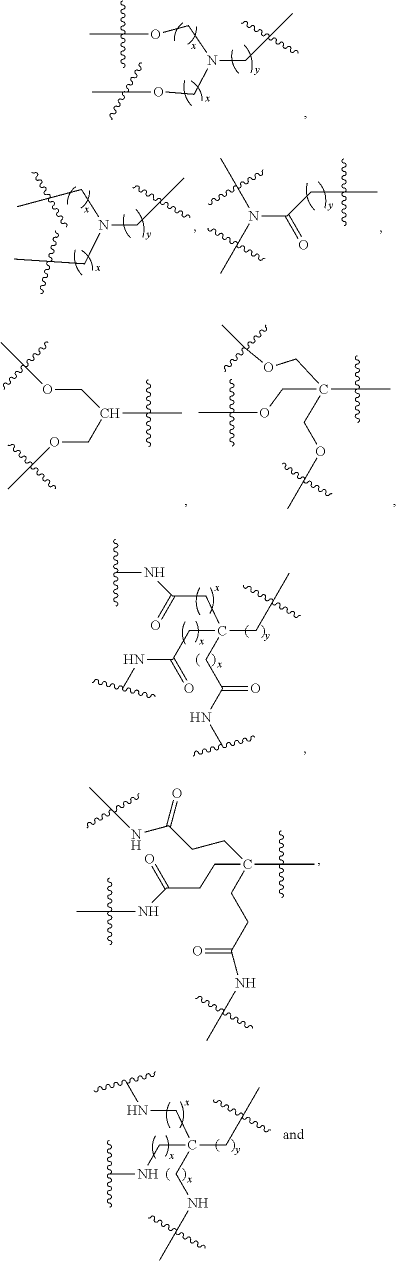

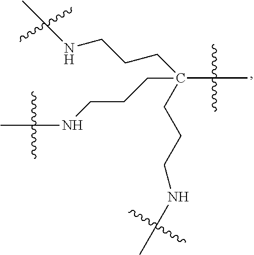

wherein each x and y are each independently 1 to 10.

Clause 116. The conjugate of any one of clauses 109 to 115, wherein: each L⁵ is independently —NHCO—C₁₋₆-alkylene-, —CONH—C₁₋₆-alkylene-, —C₁₋₆-alkylene-,

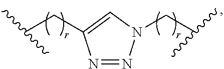

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and r, s, and t are each independently 1 to 20.

Clause 117. The conjugate of any one of clauses 109 to 116, wherein a is 1.

Clause 118. The conjugate of any one of clauses 109 to 117, wherein at least one of b, c, e, f, and g is not 0.

Clause 119. The conjugate of any one of clauses 109 to 118, wherein at least one of b or c is not 0 and at least one of e, f, and g is not 0.

Clause 120. The conjugate of any one of clauses 109 to 119, wherein a, b, and c are each independently 1 or 2.

Clause 121. The conjugate of any one of clauses 109 to 120, wherein the linker L is selected from any one of the structures of Tables 2-3.

Clause 122. The conjugate of clause 76 or 77, wherein the conjugate is selected from:
i) a conjugate derived from conjugation of a compound of any one of the structures of compound Tables described herein and a biomolecule;
ii) a conjugate derived from conjugation of a compound of any one of the structures of compound Tables described herein and a polypeptide; or
iii) a conjugate derived from conjugation of a compound of any one of the structures of compound Tables described herein and an antibody or antibody fragment.

Clause 123. The conjugate of any one of clauses 77-122, wherein the antibody or antibody fragment is an IgG antibody.

Clause 124. The conjugate of any one of clauses 77-122, wherein the antibody or antibody fragment is a humanized antibody.

Clause 125. The conjugate of any one of clauses 77-124, wherein the antibody or antibody fragment specifically binds to a secreted or soluble protein.

Clause 126. The conjugate of any one of clauses 77-124, wherein the antibody or antibody fragment specifically binds to a cell surface receptor.

Clause 127. A method of internalizing a target protein in a cell comprising a M6PR cell surface receptor, the method comprising: contacting a cellular sample comprising the cell and the target protein with an effective amount of a compound according to any one of clauses 1 to 75, or a conjugate according to any one of clauses 76 to 132, wherein the compound or conjugate specifically binds the target protein and specifically binds the cell surface receptor to facilitate cellular uptake of the target protein.

Clause 128. The method of clause 127, wherein the target protein is a membrane bound protein.

Clause 129. The method of clause 127, wherein the target protein is an extracellular protein.

Clause 130. The method of any one of clauses 127 to 129, wherein the compound or conjugate comprises an antibody or antibody fragment (Ab) that specifically binds the target protein.

Clause 131. A method of reducing levels of a target protein in a biological system, the method comprising: contacting the biological system with an effective amount of a compound according to any one of clauses 1 to 75, or a conjugate according to any one of clauses 76 to 126, wherein the compound or conjugate specifically binds the target protein and specifically binds a M6PR cell surface receptor of cells in the biological system to facilitate cellular uptake and degradation of the target protein.

Clause 134. The method of any one of clauses 131 to 133, wherein the biological system is a human subject.

Clause 135. The method of any one of clauses 131 to 133, wherein the biological system is an in vitro cellular sample.

Clause 136. The method of any one of clauses 131 to 135, wherein the target protein is a membrane bound protein.

Clause 137. The method of any one of clauses 131 to 135, wherein the target protein is an extracellular protein.

Clause 138. A method of treating a disease or disorder associated with a target protein, the method comprising: administering to a subject in need thereof an effective amount of a compound according to any one of clauses 1 to 75, or a conjugate according to any one of clauses 76 to 126, wherein the compound or conjugate specifically binds the target protein.

Clause 139. The method of clause 138, wherein the disease or disorder is an inflammatory disease.

Clause 140. The method of clause 138, wherein the disease or disorder is an autoimmune disease.

Clause 141. The method of clause 138, wherein the disease or disorder is a cancer.

Clause 151. A compound of the following formula (I):

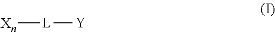

or a salt, a single stereoisomer, a mixture of stereoisomers or an isotopic form thereof, wherein:
X is a moiety that binds to a M6PR cell surface receptor;
L is a linker of the following formula:

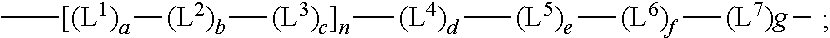

and wherein
each L¹ is independently

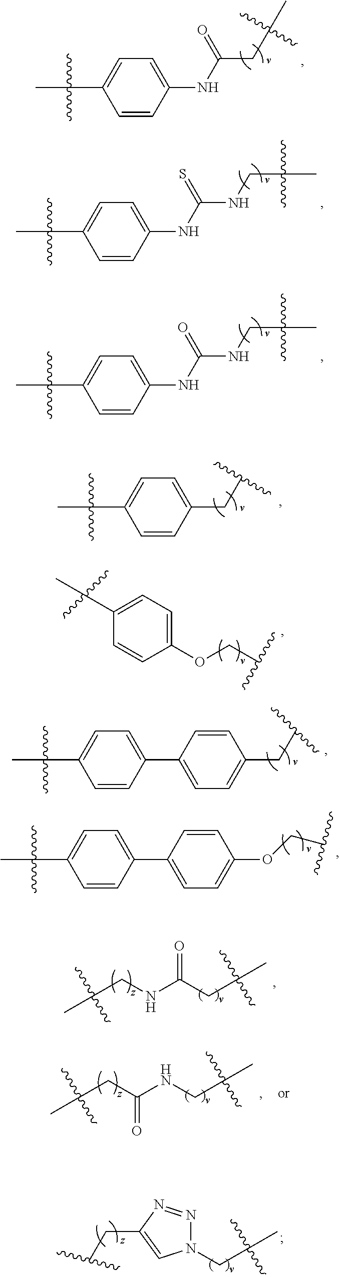

each L² is independently —C₁₋₆-alkylene-, —NHCO—C₁₋₆-alkylene-, —CONH—C₁ 6-alkylene-, —(OCH₂)ₚ—, or —(OCH₂CH₂)ₚ—;
each L³ is independently,

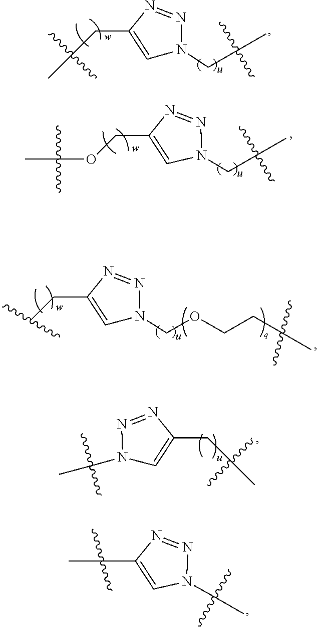

or —(OCH₂CH₂)_{q}—;
each L⁴ is independently —OCH₂CH₂—,

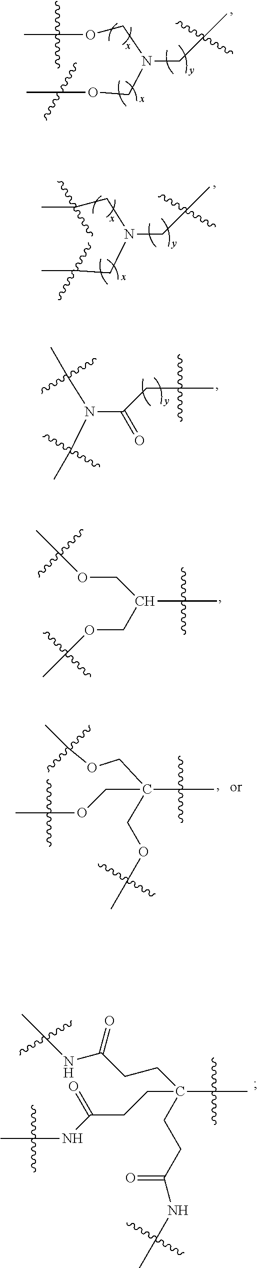

each L⁵ is independently —NHCO—C₁₋₆-alkylene-, —CONH—C₁₋₆-alkylene-, —C₁₋₆-alkylene-,

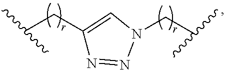

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently an integer of 1 to 10;
n is an integer of 1 to 5; wherein when d is 0, n is 1, when d is 1, n is an integer of 1 to 3, and when d is 2, n is an integer of 1 to 5;
Y is a moiety selected from the group consisting of

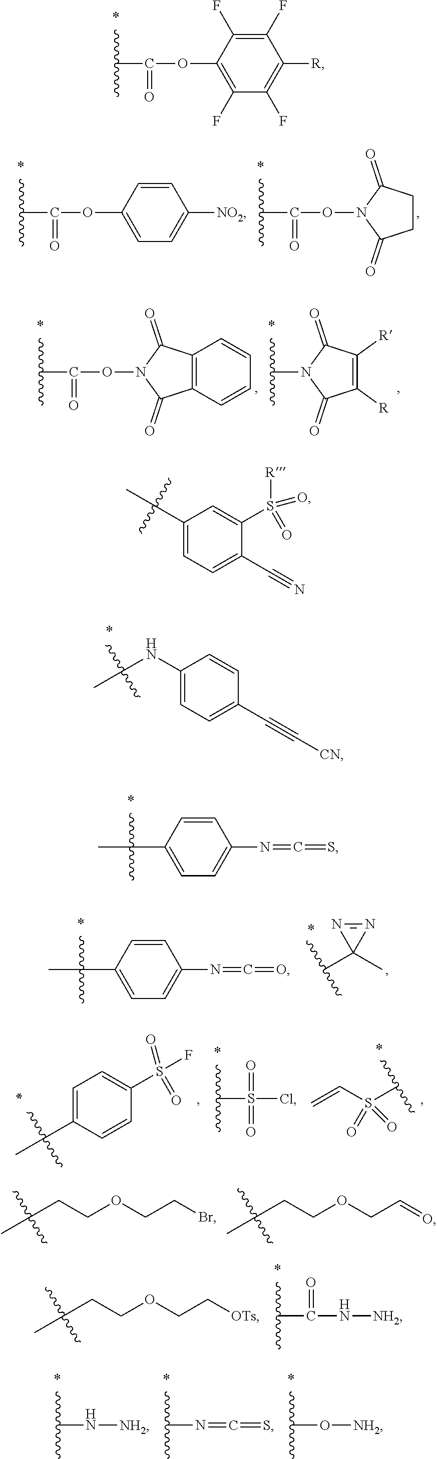

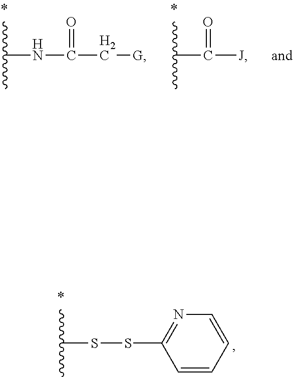

wherein [symbol] represents the point of attachment to L;
R is hydrogen or fluorine;
each R′ is independently hydrogen or halo;
G is selected from —F, —C₁, —Br, —I, —O-mesyl, and —O-tosyl;
J is selected from —C₁, —Br, —I, —F, —OH, —O—N-succinimide, —O—(4-nitrophenyl), —O-pentafluorophenyl, —O-tetrafluorophenyl, and —O—C(O)—OR¹; and R′ is —C₁-C₅ alkyl or -aryl.

Clause 154. The compound of clause 151, wherein a is 1.

Clause 155. The compound of clause 151, wherein at least one of b, c, e, f, and g is not 0.

Clause 156. The compound of clause 151, wherein at least one of b or c is not 0 and at least one of e, f, and g is not 0.

Clause 157. The compound of clause 151, wherein a, b, and c are each independently 1 or 2.

Clause 158. The compound of clause 151, wherein each X is independently selected from one of following formula:

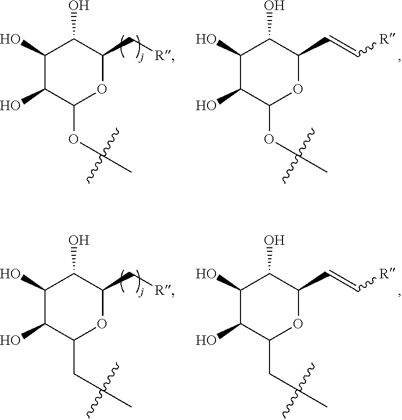

wherein,
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

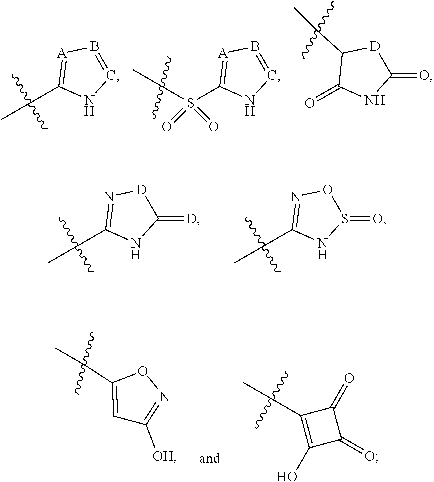

j is an integer of 1 to 3;
R¹ and R² are each independently hydrogen, halo, or CN;
R³ and R⁴ are each independently C₁₋₆ alkyl;
A, B, and C are each independently CH or N; and
D is each independently O or S.

Clause 159. The compound of clause 151, wherein each X is independently selected from one of following formula:

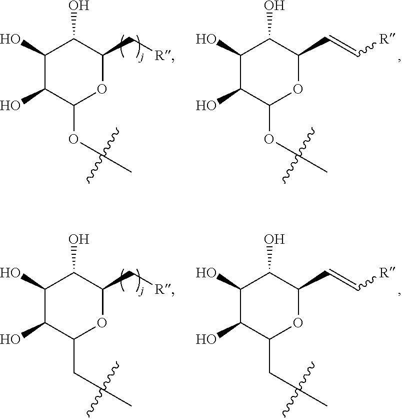

wherein
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

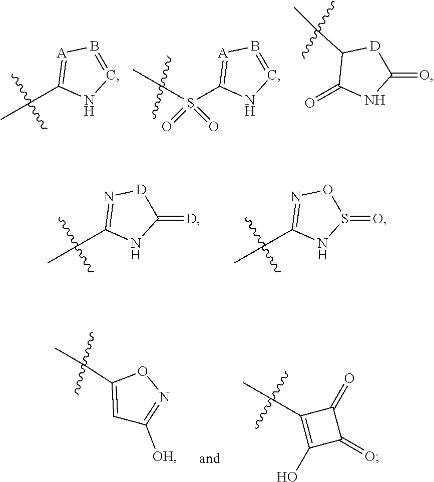

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S.

Clause 161. A conjugate of the following formula:

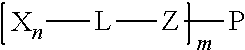

or a pharmaceutically acceptable salt thereof,
wherein:
X is a moiety that binds to a M6PR cell surface receptor;
L is a linker of the following formula:

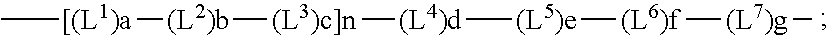

and
wherein
each L¹ is independently

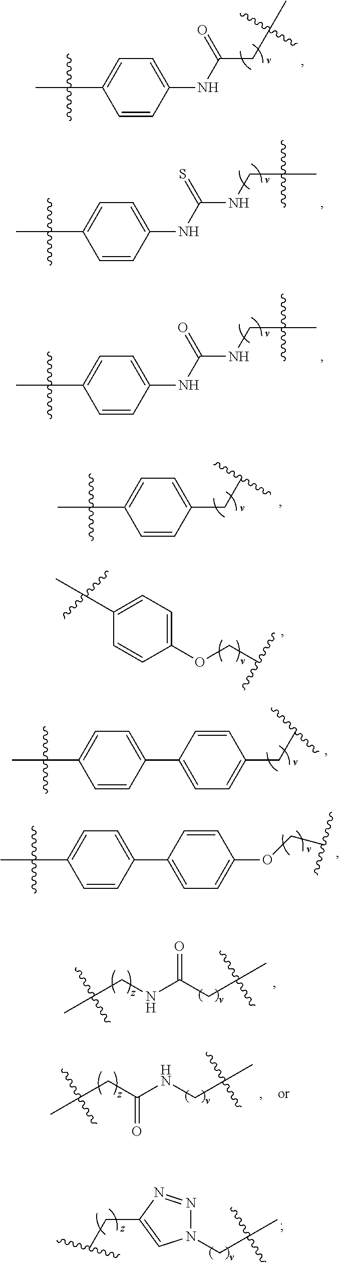

each L² is independently —C₁ 6-alkylene-, —NHCO—C₁₋₆-alkylene-, —CONH—C₁₋₆-alkylene-, —(OCH₂)ₚ—, or —(OCH₂CH₂)ₚ—;
each L³ is independently

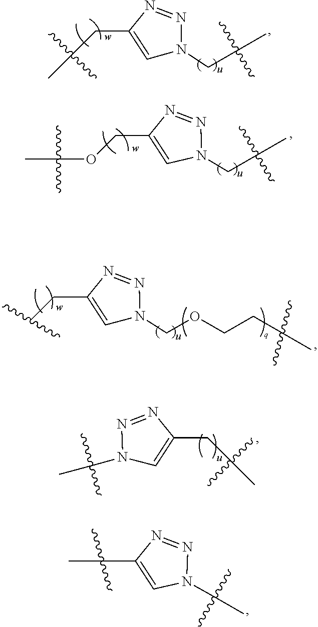

or —(OCH₂CH₂)_{q}—;

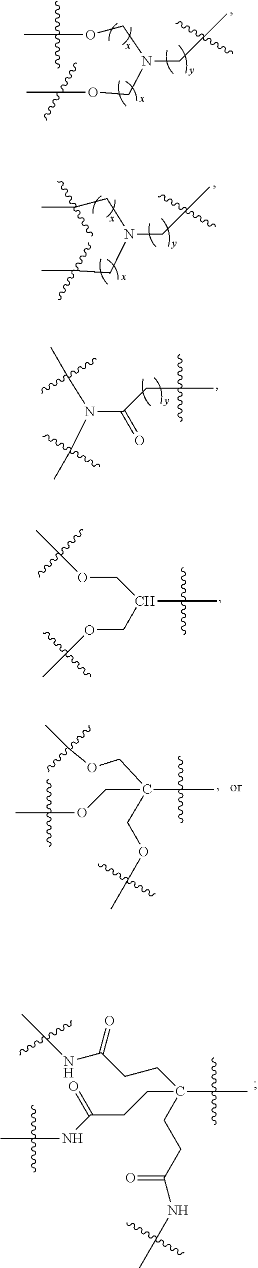

each L⁴ is independently —OCH₂CH₂—,
each L⁵ is independently —NHCO—C₁ 6-alkylene-, —CONH—C₁₋₆-alkylene-, —C₁₋₆-alkylene-,

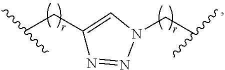

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently an integer of 1 to 10;
n is an integer of 1 to 5; wherein when d is 0, n is 1, when d is 1, n is an integer of 1 to 3, and when d is 2, n is an integer of 1 to 5;
Z is selected from the group consisting of

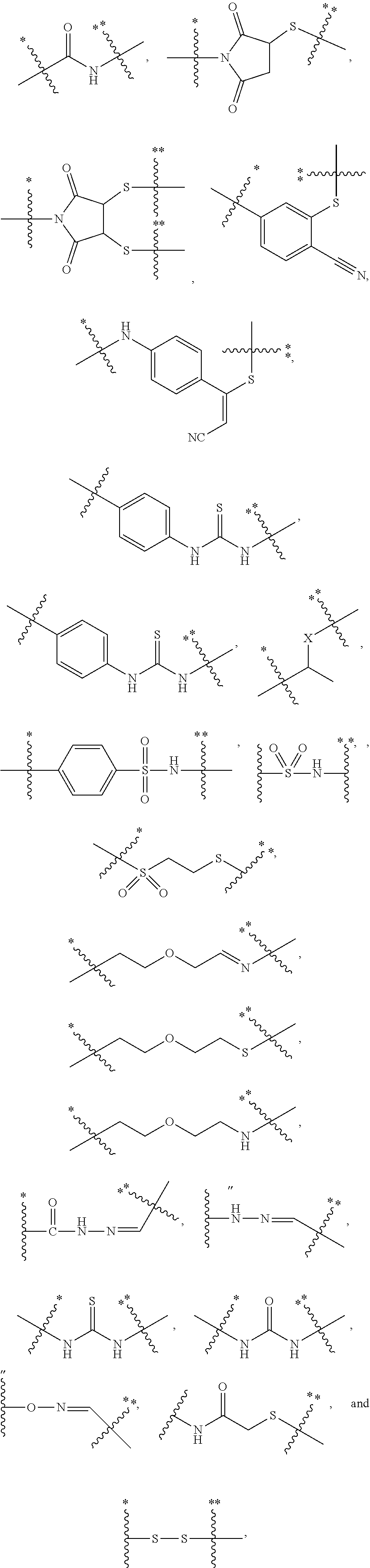

wherein [symbol] represents the point of attachment to L,
wherein [symbol] represents the point of attachment to P,
X is CH₂, NH, O or S; and
P is a polypeptide.

Clause 162. The conjugate of clause 161, wherein P comprises an antibody or an antigen-binding fragment of an antibody.

Clause 163. A conjugate of the following formula:

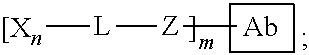

or a pharmaceutically acceptable salt thereof,
wherein:
X is a moiety that binds to a M6PR cell surface receptor;
L is a linker of the following formula:

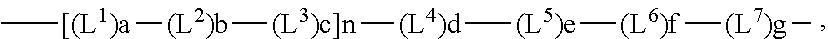

and
wherein
each L¹ is independently

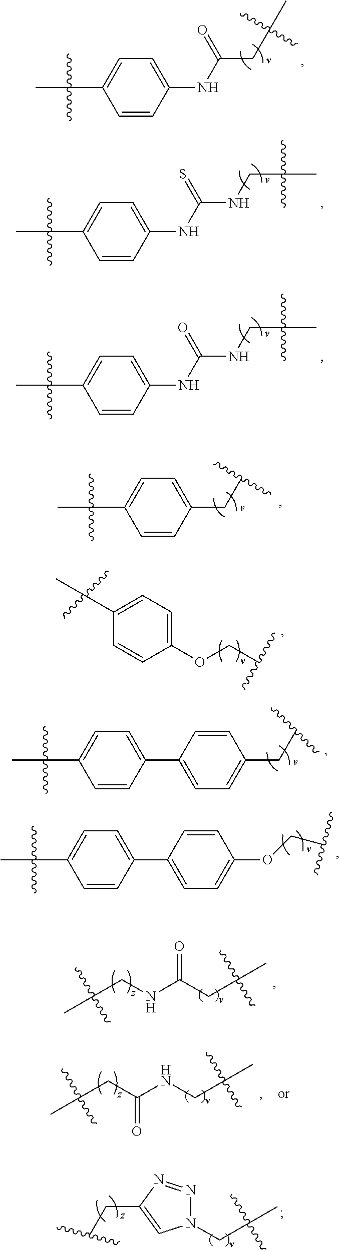

each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

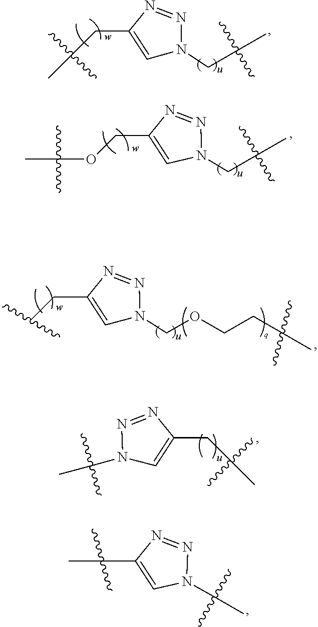

or —(OCH₂CH₂)_{q}—;
each L⁴ is independently —OCH₂CH₂—,

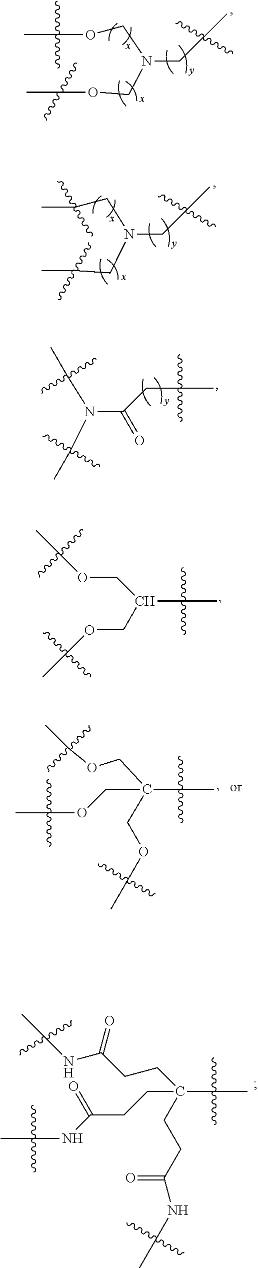

each L⁵ is —NHCO—C₁₋₆-alkylene-, —CONH—C₁₋₆-alkylene-, —C₁₋₆-alkylene-,

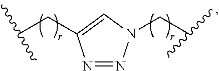

or —(OCH₂CH₂)—;
each L⁶ is —NHCO—C₁ 6-alkylene-, —CONH—C₁₋₆-alkylene-, —C₁₋₆-alkylene-, or —(OCH₂CH₂)ₛ—;
each L⁷ is —NHCO—C₁ 6-alkylene-, —CONH—C₁₋₆-alkylene-, —C₁₋₆-alkylene-, —(OCH₂CH₂)ₜ—, or —OCH₂—;
p, q, r, s, and t are each independently an integer of 1 to 20; a is 1 or 2; b, c, d, e, f, and g are each independently 0, 1, or 2; u, v, w, x, y, and z are each independently 1, 2, 3, 4, 5, or 6;
n is an integer of 1 to 5; wherein when d is 0, n is 1, when d is 1, n is an integer of 1 to 3, and when d is 2, n is an integer of 1 to 5;
m is an integer from 1 to 8;
Z is selected from the group consisting of

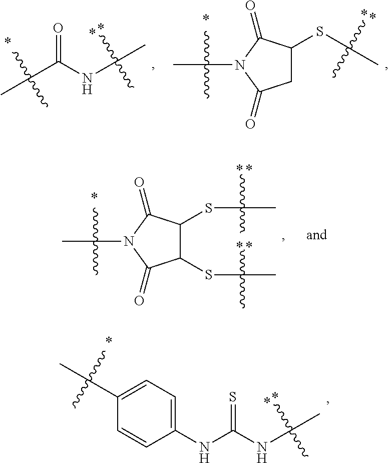

wherein [symbol] represents the point of attachment to L, wherein [symbol] represents the point of attachment to [symbol] ; and
[symbol] is an antibody.

Clause 166. The conjugate of any one of clauses 161-165, wherein each X is independently selected from one of following formula:

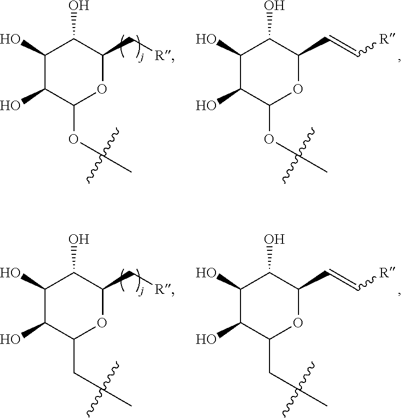

wherein,
R″ is selected from the group consisting of —OH, —CR¹R²OH, —P═O(OH)₂, P(═O)ROH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

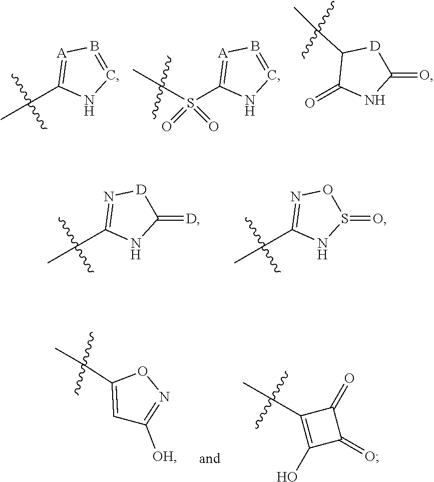

j is an integer of 1 to 3;
R¹ and R² are each independently hydrogen, halo, or CN;
R³ and R⁴ are each independently C₁₋₆ alkyl;
A, B, and C are each independently CH or N; and
D is each independently O or S.

Clause 167. The conjugate of any one of clauses 161-165, wherein each X is independently selected from one of following formula:

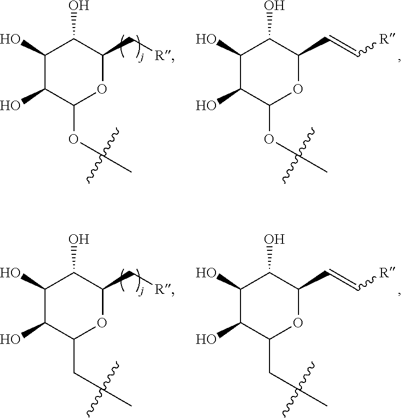

wherein
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

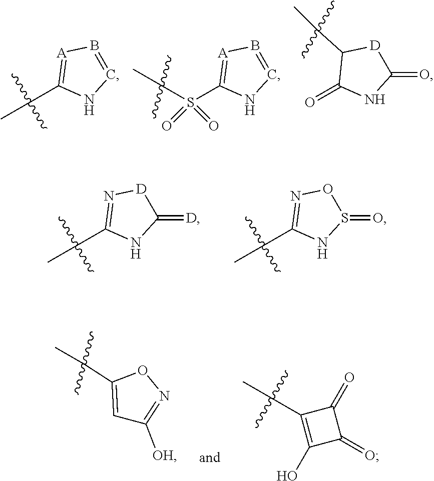

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S.

Clause 169. A pharmaceutical composition comprising the conjugate or pharmaceutically acceptable salt of any one of clauses 161-168, and a pharmaceutically acceptable carrier.

Clause 170. The pharmaceutical composition of clause 169, wherein m is an integer of 4 to 8.

Clause 171. The pharmaceutical composition comprising the conjugate or pharmaceutically acceptable salt of clause 170, wherein m is 4.

Clause 172. The conjugate of any one of clauses 163-168, wherein the antibody is an IgG antibody.

Clause 173. The conjugate of any one of clauses 163-168, wherein the antibody is a humanized antibody.

Clause 174. The conjugate of any one of clauses 163-168, wherein the antibody specifically binds to a secreted or soluble protein.

Clause 175. The conjugate of any one of clauses 163-168, wherein the antibody specifically binds to a cell surface receptor.

Clause 176. The conjugate of any one of clauses 163-168, wherein the antibody specifically binds to programmed death ligand-1 (PD-L1) protein.

Clause 177. The conjugate of any one of clauses 163-168, wherein the antibody specifically binds to Vascular Endothelial Growth Factor (VEGF) protein.

Clause 178. The conjugate of any one of clauses 163-168, wherein the antibody specifically binds to a Fibroblast Growth Factor Receptor 2 (FGFR2) protein or a Fibroblast Growth Factor Receptor 3 (FGFR3) protein.

Clause 179. The conjugate of any one of clauses 163-168, wherein the antibody is cetuximab.

Clause 180. The conjugate of any one of clauses 163-168, wherein the antibody is matuzumab.

Clause 181. The conjugate of any one of clauses 163-168, wherein the antibody is atezolizumab.

Clause 182. A method of treating a disease or disorder by administering to a subject in need thereof an effective amount of the conjugate or pharmaceutically acceptable salt of any one of clauses 163-168 or the pharmaceutical composition of clause 169.

Clause 183. The method of clause 182, wherein the disease or disorder is an inflammatory disease.

Clause 184. The method of clause 182, wherein the disease or disorder is an autoimmune disease.

Clause 185. The method of clause 182, wherein the disease or disorder is a cancer.

### 5. EXAMPLES

The examples in this section are offered by way of illustration, and not by way of limitation.

### 5.1. Preparation of Compounds

The following are illustrative schemes and examples of how the compounds described herein can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

Synthetic methods for preparing M6PR binding moieties, precursors thereof, and conjugates thereof, which can be adapted for use in the preparing compounds, and synthons thereof, of this disclosure are described in International Application No. PCT/US2021/012846, published as WO2021/142377, and PCT publication WO2020132100, the disclosures of which are herein incorporated by reference in their entirety.

### 5.1.1. Preparation of M6PR Binding Moiety Synthons

### Synthons A-10 and Compound A. Synthesis of (2-((2R,3S,4S,5S,6R)-3,4,5-trihydroxy-6-(4-isothiocyanatophenoxy)tetrahydro-2H-pyran-2-yl)ethyl)phosphonic acid (Compound A)

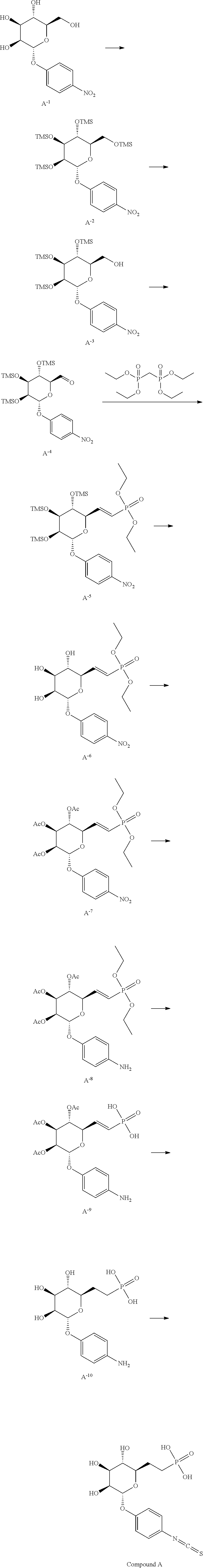

### (((2R,3S,4S,5R,6R)-2-(4-nitrophenoxy)-6(((trimethylsilyl)oxy)methyl)tetrahydro-2H-pyran-3,4,5-triyl)tris(oxy))tris(trimethylsilane) (A-2)

A solution of (2R,3S,4S,5S,6R)-2-(hydroxymethyl)-6-(4-nitrophenoxy)tetrahydro-2H-pyran-3,4,5-triol (A-1) (1.0 eq, 26.0 g, 86.37 mmol) in DMF (500 mL) was cooled to 0° C. Then triethylamine (6.4 eq, 288 mL, 552.0 mmol) and trimethylsilyl chloride (24.0 eq 70 mL, 2071.0 mmol) were added under nitrogen atmosphere to above solution. The resulting mixture was stirred at room temperature under nitrogen for 24 h. The reaction mixture was partitioned between ethyl acetate and water. The water layer was extracted again with ethyl acetate. The combined organic layers were dried over sodium sulfate, filtered, and purified via silica gel chromatography (0 to 5% ethyl acetate in hexane) to afford Intermediate A-2 as colorless oil. Yield: 36.8 g (72.3%); ¹H NMR (400 MHz, CDCl₃) δ 8.18 (dd, J=12.36, 3.16 Hz, 2H), 7.16 (dd, J=12.4, 3.12 Hz, 2H), 5.37 (d, J=2.36 Hz, 1H), 3.99-3.87 (m, 3H), 3.72-3.69 (m, 2H), 3.50-3.48 (m, 1H), 0.2-0.07 (m, 36H).

### ((2R,3R,4S,5S,6R)-6-(4-nitrophenoxy)-3,4,5-tris((trimethylsilyl)oxy)tetrahydro-2H-pyran-2-yl)methanol (A-3)

To a stirred solution of Intermediate A-2 (1.0 eq, 10.0 g, 16.97 mmol) in mixture of DCM: methanol (8: 2 ratio, 100 mL) ammonium acetate (1.5 eq, 1.96 g, 25.46 mmol) was added at room temperature under nitrogen. The resulting mixture was stirred at room temperature under nitrogen for 16 h. The reaction mixture was partitioned between ethyl acetate and water. The water layer was extracted again with ethyl acetate. The combined organic layers were dried over sodium sulfate, filtered, concentrated under vacuum and purified via silica gel chromatography (20-30% ethyl acetate in hexane) to afford Intermediate A-3 as white solid. Yield: 7.0 g (80%); LC-MS m/z 516.13 [M−1]⁻.

### (2S,3R,4S,5S,6R)-6-(4-nitrophenoxy)-3,4,5-tris((trimethylsilyl)oxy)tetrahydro-2H-pyran-2-carbaldehyde (A-4)

To a stirred solution of oxalyl chloride (1.1 eq, 0.5 mL, 5.31 mmol) in DCM (5 mL) at −78° C. was added a solution of DMSO (2.2 eq, 0.76 mL, 10.62 mmol) in DCM (5 mL) over 5 min. After being stirred at −78° C. for 20 min, a solution of Intermediate A-3 (1.0 eq, 2.5 g, 4.83 mmol) in DCM (10 mL) was added to the mixture. The reaction mixture was further stirred at −78° C. for 60 min, followed by addition of triethylamine (5.0 eq, 3.4 mL, 24.15 mmol). The resulting mixture was allowed to reach room temperature over 1 h. The turbid mixture was diluted with DCM and washed with water followed by brine solution. The organic layer was dried over sodium sulfate, filtered, and concentrated under high vacuum to afford Intermediate A-4 as light brown gel (2.2 g, crude), which was used without further purification for the next step.

### Diethyl ((E)-2-((2R,3R,4S,5S,6R)-6-(4-nitrophenoxy)-3,4,5-tris((trimethylsilyl)oxy)tetrahydro-2H-pyran-2-yl)vinyl)phosphonate (A-5)

A stirred suspension of tetraethyl methylenebis(phosphonate) (1.5 eq, 1.85 g, 6.40 mmol) in dry THF (20 mL) was cooled to −78° C. and added n-BuLi in hexane 2.0 M (1.25 eq, 2.6 ml, 5.33 mmol). The resulting mixture was stirred for 1 h at −78° C., then Intermediate A-4 (1.0 eq, 2.2 g, 4.27 mmol) in dry THF (10 mL) was added at −78° C. The bath was removed and the reaction mixture was allowed to room temperature and stirring continued for 12 h. A saturated aqueous solution of NH₄Cl was added and extracted with ethyl acetate. Ethyl acetate layer washed with water followed by saturated brine solution. The organic layer was dried over sodium sulfate, filtered and concentrated. The crude was purified via silica gel chromatography (30-40% ethyl acetate in hexane) to afford Intermediate A-5 as colorless gel. Yield (1.3 g, 48%); LC-MS m/z 650.57 [M+1]⁺.

### Diethyl ((E)-2-((2R,3S,4S,5S,6R)-3,4,5-trihydroxy-6-(4-nitrophenoxy)tetrahydro-2H-pyran-2-yl)vinyl)phosphonate (A-6)

To a stirred solution of Intermediate A-5 (1.0 eq, 1.3 g, 1.54 mmol) in methanol (15 mL). was added Dowex 50WX8 hydrogen form at room temperature under nitrogen atmosphere. The resulting mixture was stirred at room temperature under nitrogen for 2 h. The reaction mixture filtered and washed with methanol, filtrate concentrated under vacuum to afford diethyl ((E)-2-((2R,3S,4S,5S,6R)-3,4,5-trihydroxy-6-(4-nitrophenoxy)tetrahydro-2H-pyran-2-yl)vinyl)phosphonate (6) as white solid. Yield: 0.78 g (90%); LC-MS m/z 434.17 [M+1]⁺.

### (2R,3R,4S,5S,6R)-2-((E)-2-(diethoxyphosphoryl)vinyl)-6-(4-nitrophenoxy)tetrahydro-2H-pyran-3,4,5-triyl triacetate (A-7)

To a stirred solution of Intermediate A-6, (1.00 eq, 0.78 g, 1.80 mmol) in pyridine (10 mL) was added an acetic anhydride (10.0 eq, 1.8 mL, 18.0 mmol) dropwise at 0° C. under nitrogen. The cold bath was removed and the resulting mixture was stirred at room temperature under nitrogen for 16 h. Pyridine was removed on a high vacuum and the residue was partitioned between ethyl acetate and aqueous 1N HCl. The water layer was extracted again with ethyl acetate. The combined organic layers were dried over sodium sulfate, filtered, concentrated and purified via silica gel chromatography (2.5% methanol in dichloromethane) to afford Intermediate A-7 as white solid. Yield: 1.0 g (100%); LC-MS m/z 560.17 [M+1]⁺.

### (2R,3S,4S,5R,6R)-2-(4-aminophenoxy)-6-(2-(diethoxyphosphoryl)ethyl)tetrahydro-2H-pyran-3,4,5-triyl triacetate (A-8)

To a stirred solution of Intermediate A-7 (1.0 eq, 1.0 g, 1.78 mmol) in methanol (15 mL) 10% palladium on carbon (0.200 g) was added at room temperature under nitrogen. The resulting mixture was stirred at room temperature under hydrogen gas pressure (100 psi) for 16 h. The reaction mixture filtered through Celite bed and washed with methanol, filtrate concentrated under vacuum to afford Intermediate A-8 as brown sticky gel. Yield: 0.700 g (73.6%); LC-MS m/z 532.21 [M+1]⁺.

### (2-((2R,3R,4S,5S,6R)-3,4,5-triacetoxy-6-(4-aminophenoxy)tetrahydro-2H-pyran-2-yl)ethyl)phosphonic acid (A-9)

To a stirred solution of Intermediate A-8 (1.00 eq, 2.0 g, 5.73 mmol) in acetonitrile (15 mL) bromotrimethylsilane (5.0 eq, 3.8 mL, 28.65 mmol) was added dropwise at 0° C. under nitrogen. The cold bath removed and the resulting mixture was stirred at room temperature under nitrogen for 16 h. Volatiles were removed on a rotary evaporator and the residue was dried under high vacuum. The crude residue was triturated with diethyl ether and dried under high vacuum to afford Intermediate A-9 as brown solid. Yield: 2.2 g, crude. LC-MS m/z 476.0 [M+1]⁺.

### (2-((2R,3S,4S,5S,6R)-6-(4-aminophenoxy)-3,4,5-trihydroxytetrahydro-2H-pyran-2-yl)ethyl)phosphonic acid (A-10)

To a stirred solution of Intermediate A-9 (1.0 eq, 2.0 g, 4.21 mmol) in mixture of methanol:water (8:2, 15 mL) triethylamine (5.0 eq, 2.93 mL, 21.05 mmol) was added dropwise at 0° C. under nitrogen. The cold bath removed and the resulting mixture was stirred at room temperature for 16 h. Methanol was removed on a rotary evaporator and the residue was dried under high vacuum. The residue was taken up in water and purified via preparatory HPLC (2-10% acetonitrile in water with 5 mM ammonium acetate). Fractions containing the desired product were combined and lyophilized to dryness to afford Intermediate A-10 as brown solid. Yield: 0.350 g (25%); LC-MS m/z 348.0 [M−H]⁻.

### (2-((2R,3S,4S,5S,6R)-3,4,5-trihydroxy-6-(4-isothiocyanatophenoxy)tetrahydro-2H-pyran-2-yl)ethyl)phosphonic acid (Compound A)

To a stirred solution of Intermediate A-10 (1.0 eq, 1.75 g, 5.01 mmol) in mixture of ethanol:water (7:3) (20 ml) was added thiophosgene (5.00 eq, 1.92 mL, 25.05 mmol) dropwise at 0° C. under nitrogen. The cold bath removed and the resulting mixture was stirred at room temperature under nitrogen for 3 h. Volatiles were removed on a rotary evaporator and the residue was dried under high vacuum. The residue was taken up in water and purified via prep-HPLC (20-40% acetonitrile in water with 5.0 mmol ammonium acetate). Fractions containing the desired product were combined and lyophilized to dryness to afford Compound A as a white solid. Yield: 0.135 g (6.8%) LC-MS m/z 392.08 [M+1]+; H NMR (400 MHz, D₂O) δ 7.32 (d, J=8.92 Hz, 2H), 7.12 (d, J=8.96 Hz, 2H), 5.57 (s, 1H), 4.13 (s, 1H), 3.96 (dd, J=9.16, 3.44 Hz, 1H), 3.59-3.48 (m, 2H), 2.03-1.88 (m, 1H), 1.68-1.54 (m, 2H), 1.27-1.15 (m, 1H).

### Preparation of Synthon 8D

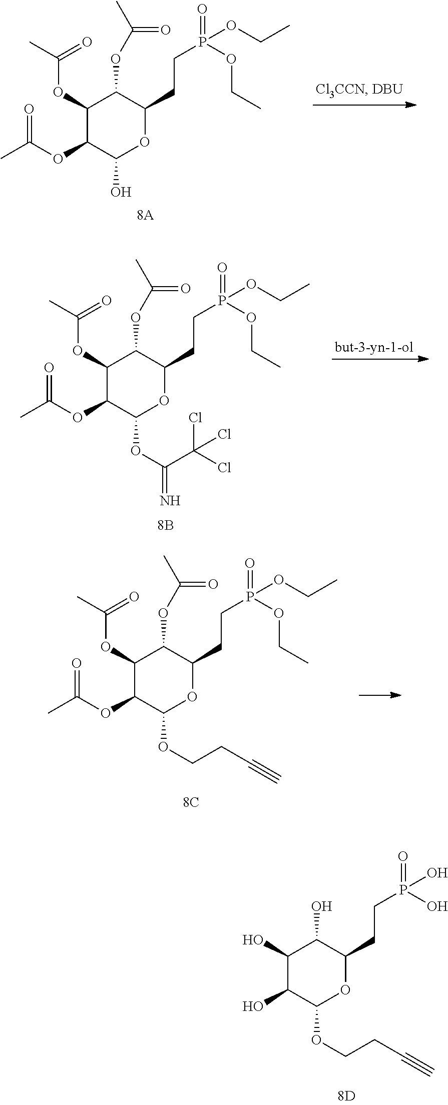

DBU (0.05 eq, 0.025 mL, 0.168 mmol) was added to a stirred solution of (2R,3R,4S,5S,6S)-2-(2-(diethoxyphosphoryl)ethyl)-6-hydroxytetrahydro-2H-pyran-3,4,5-triyl triacetate (8A) (1.00 eq, 1.48 g, 3.36 mmol) and trichloroacetonitrile (10.0 eq, 3.4 mL, 33.6 mmol) in DCM (30 mL) at 0° C. under nitrogen. The resulting mixture was stirred at 0° C. under nitrogen. More DBU (0.0500 eq, 0.025 mL, 0.168 mmol) was added and the cold bath was removed. The resulting mixture was stirred at room temperature for 45 min. Most of the solvent was removed on a rotary evaporator. The residue was loaded onto a silica gel loading column which was pre-equilibrated with 0.1% triethylamine in dichloromethane and purified via silica gel chromatography (column pre-equilibrated with 0.1% triethylamine in 30% ethyl acetate/hexanes) (30-100% ethyl acetate in hexanes). Fractions containing the desired product were combined and concentrated on a rotary evaporator. The residue was stripped down from dry dichloromethane twice, dried under high vacuum for 30 min, and then stored under nitrogen at −80° C. to afford Compound 8B as a colorless semi-solid. Yield: 1.26 g, 64%; H NMR (300 MHz, Chloroform-d) δ 8.74 (s, 1H), 6.21 (s, 1H), 5.45 (s, 1H), 5.34 (t, J=11.2 Hz, 1H), 5.20 (t, J=10.0 Hz, 1H), 4.16-4.00 (m, 4H), 4.00-3.88 (m, 1H), 2.18 (s, 3H), 2.07 (s, 3H), 2.00 (s, 3H), 1.95-1.64 (m, 4H), 1.31 (t, J=7.3 Hz, 6H).

Compound 8B (1.00 eq, 1.25 g, 2.14 mmol) was dissolved in dry DCM (10 mL) with stirring under nitrogen. But-3-yn-1-ol (2.00 eq, 0.32 mL, 4.28 mmol) was added and the resulting mixture was cooled to −78° C. with stirring under nitrogen. A solution of boron trifluoride diethyl etherate (0.500 eq, 0.13 mL, 1.07 mmol) in dichloromethane (5 mL) was added slowly. The −78° C. cold bath was removed and the reaction mixture was allowed to slowly warm under nitrogen for 50 min. The reaction mixture was cooled with a water/ice bath and allowed to stir an additional 30 min at 0° C. under nitrogen and then worked up. The reaction mixture was partitioned between dichloromethane and saturated aqueous sodium bicarbonate. The water layer was extracted again with dichloromethane. The combined organics were dried over sodium sulfate, filtered, and purified via silica gel chromatography (20-100% ethyl acetate in dichloromethane) to afford Compound 8C as a colorless viscous oil. Yield: 408 mg, 39%; LC-MS m/z 493.4 [M+1]+; ¹H NMR (300 MHz, Chloroform-d) δ 5.35-5.19 (m, 2H), 5.09 (t, J=9.9 Hz, 1H), 4.79 (s, 1H), 4.21-3.98 (m, 4H), 3.91-3.68 (m, 2H), 3.64-3.50 (m, 1H), 2.55-2.44 (m, 2H), 2.15 (s, 3H), 2.05 (s, 3H), 1.98 (s, 3H), 2.07-1.62 (m, 5H), 1.32 (t, J=7.2 Hz, 6H).

Bromotrimethylsilane (5.00 eq, 0.47 mL, 3.57 mmol) was added slowly to a stirred solution of Compound 8C (1.00 eq, 352 mg, 0.715 mmol) in MeCN (7 mL) at 0° C. under nitrogen. The cold bath was removed and the resulting mixture was stirred at room temperature under nitrogen for 3.5 h. Volatiles were removed on a rotary evaporator and the residue was dried under high vacuum briefly. The residue was dissolved in methanol (7 mL) with stirring under nitrogen and sodium methoxide (25 wt % in methanol) (2.50 eq, 0.41 mL, 1.79 mmol) was added. The resulting mixture was stirred at room temperature under nitrogen for 1 h. Acetic acid (3.00 eq, 0.12 mL, 2.14 mmol) was added and then volatiles were removed on a rotary evaporator. The residue was taken up in water and purified via preparatory HPLC (0-15% acetonitrile in water with 0.1% TFA). Most of the solvent was removed on a rotary evaporator at 30° C. and then the remainder was lyophilized to dryness to afford Compound 8D as a white solid. Yield: 208 mg, 94%; LC-MS m/z 311.3 [M+1]+; ¹H NMR (300 MHz, Deuterium Oxide) δ 4.88-4.80 (m, 1H), 3.93 (s, 1H), 3.84-3.70 (m, 2H), 3.70-3.56 (m, 2H), 3.48 (t, J=9.7 Hz, 1H), 2.57-2.44 (m, 2H), 2.37 (s, 1H), 2.15-1.61 (m, 4H).

### Synthesis of Compound B

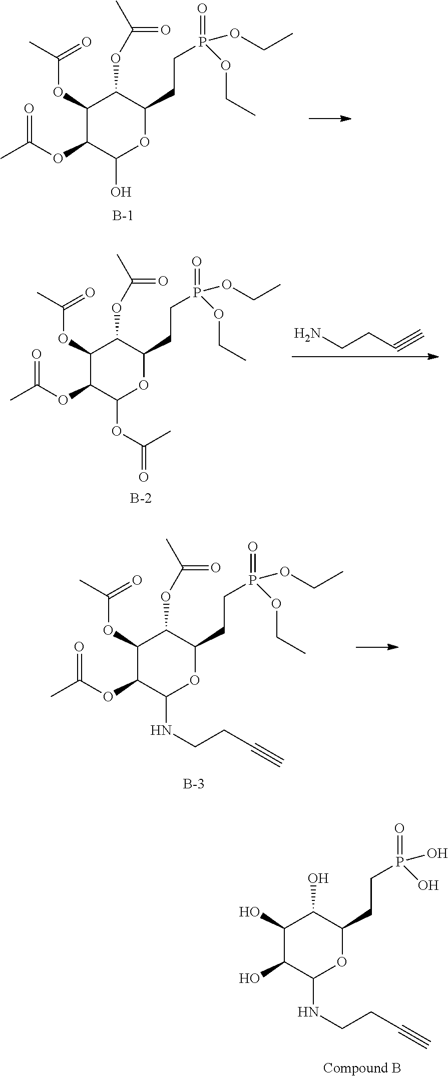

Compound B is synthesized employing the procedures described for Compound 8D using but-3-yn-1-amine in lieu of but-3-yn-1-ol.

Alternatively, Intermediate B-2 may be prepared by addition of pyridine to a solution of Intermediate B-1 in excess acetic anhydride. The resulting mixture is stirred at 20° C. for 16 h. The reaction solution is concentrated in vacuo and the residual pyridine is removed by azeotropic distillation with toluene followed by high vacuum drying to afford Intermediate B-2.

Other M6PR binding moiety synthons including an amino linking group at the 1-position of the pyranose ring can be prepared by adapting the methods shown.

### Synthesis of Synthon 38C

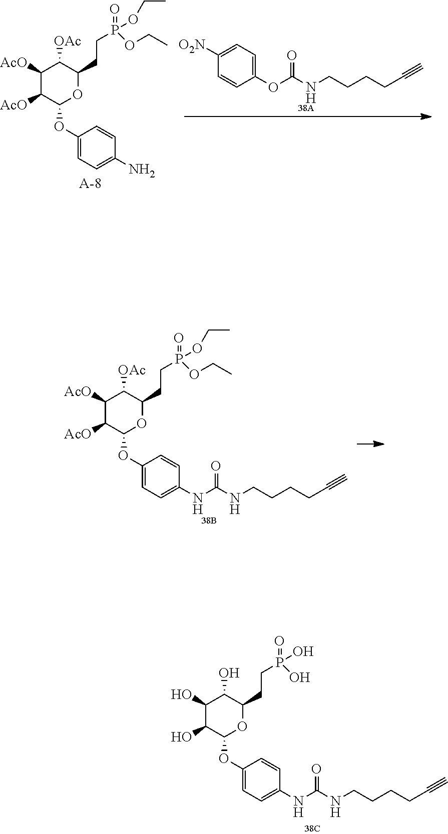

To a round bottom flask containing Intermediate A-8 (1.00 eq, 218 mg, 0.398 mmol) was added (4-nitrophenyl)N-hex-5-ynylcarbamate (38A) (1.80 eq, 188 mg, 0.717 mmol) and anhydrous DCM (4 mL). To the reaction solution was added triethylamine (2.08 eq, 0.11 mL, 0.826 mmol) and the solution was allowed to stir at 40° C. for 16 hr. The reaction mixture was then diluted with dichloromethane (30 mL) and washed with aq. NaOH, water, and brine. The organic layer was dried over anhydrous MgSO₄, filtered, and concentrated in vacuo. The residue was purified by column chromatography on silica gel, eluting with methanol/chloroform to afford Compound 38B. Yield: 154 mg, 58%); LCMS m/z 655.6 [M+1]⁺.

To a nitrogen-purged round bottom flask containing Compound 38B (1.00 eq, 170 mg, 0.260 mmol) was added acetonitrile (4 mL). The solution was allowed to cool to 0° C. under nitrogen prior to dropwise addition of TMSBr (5.00 eq, 0.18 mL, 1.30 mmol). The cold bath was removed and the resulting mixture was stirred at room temperature under nitrogen. LCMS at 2 h shows no SM remaining and product M+H=599.6 observed. The solvent was removed on a rotary evaporator and the residue was dried under high vacuum. The resulting intermediate, 2-[(2R,3R,4S,5S,6R)-3,4,5-triacetoxy-6-[4-(hex-5-ynylcarbamoylamino)phenoxy]tetrahydropyran-2-yl]ethylphosphonic acid (155 mg, 0.259 mmol, 99.72% yield), was dissolved in methanol (3 mL). To the stirring solution under nitrogen was added NaOMe, 25 wt % in MeOH (2.50 eq, 0.14 mL, 0.649 mmol). The resulting mixture was stirred at room temperature under nitrogen for 50 min. LCMS found mostly starting material remains. Another aliquot of NaOMe, 25 wt % in MeOH (2.50 eq, 0.14 mL, 0.649 mmol) was added and allowed to stir at 20° C. for 1 hr longer. Acetic acid (13.5 eq, 0.20 mL, 3.50 mmol) was added, and solvents were removed on a rotary evaporator. The residue was taken up in DMSO and purified via preparatory HPLC (0-35% acetonitrile in water with 0.1% TFA). The purified product fractions were combined and lyophilized to dryness to afford Compound 38C as a white solid. Yield: 45 mg, 37%; LCMS m/z 473.6 [M+1]⁺.

### Synthesis of Synthon 39B/53A

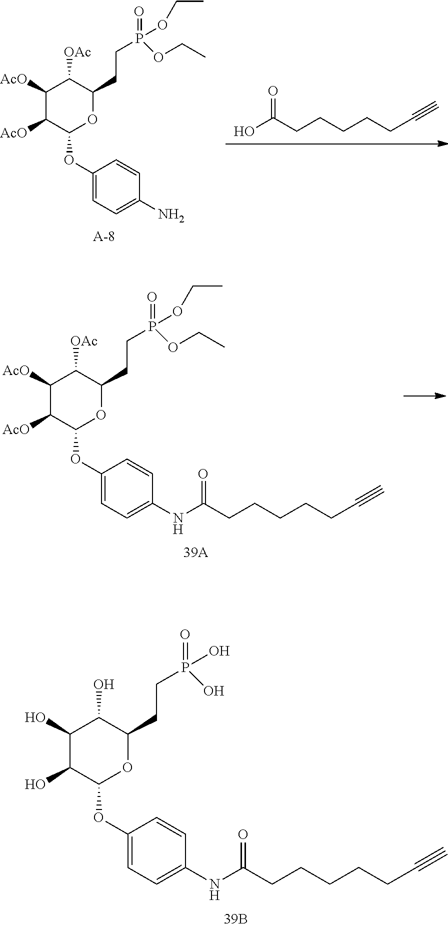

To a nitrogen-purged round bottom flask was added oct-7-ynoic acid (1.66 eq, 82.6 mg, 0.589 mmol), DMF (3 mL), and HATU (1.50 eq, 203 mg, 0.534 mmol). The reaction solution was allowed to stir at 20° C. for 20 min prior to the addition of Intermediate A-8 (1.00 eq, 195 mg, 0.356 mmol) in 1 mL of DMF. The reaction solution was allowed to stir 24 hr at 20° C. prior to analysis by LCMS. The reaction solution was diluted with EtOAc (30 mL) and washed with aq. Sat. NH₄Cl (20 mL) and then aq. sat. NaCl (20 mL). The partitioned EtOAc phase was dried over Na₂SO₄, filtered, and concentrated in vacuo to afford crude product that purified by column chromatography on silica gel using a mobile phase of 100% Hx to 750% EtOAc/Hx over 15 min to afford Compound 39A. Yield: 182 mg, 76%; LCMS m/z 653.6 [M+1]⁺.

To a nitrogen-purged round bottom flask containing Compound 39A (1.00 eq, 182 mg, 0.278 mmol) and anhydrous acetonitrile (1 mL) at 0° C. was added TMSBr (5.00 eq, 0.18 mL, 1.39 mmol) under nitrogen. The cold bath was removed and the resulting mixture was stirred at room temperature under nitrogen for 3.5 h. LCMS analysis shows no starting reagent remaining. Volatiles were removed on a rotary evaporator and the residue was dried under high vacuum briefly. The residue was dissolved in methanol (1 mL) with stirring under nitrogen and sodium methoxide, 25 wt % in MeOH (2.50 eq, 0.15 mL, 0.696 mmol) was added. The resulting mixture was stirred at room temperature under nitrogen for 30 min. To the reaction mixture was added Acetic Acid (5.00 eq, 0.080 mL, 1.39 mmol), and the volatiles were removed in vacuo. The residue was taken up in DMSO and purified via reverse-phase preparatory HPLC (0-35% acetonitrile in water with 0.1% TFA) to afford purified fractions. The combined fractions were lyophilized to dryness to afford Compound 39B as a white solid. Yield: 65 mg, 50%; LCMS m/z 472.3 [M+1]⁺.

### Synthesis of Synthon 49B

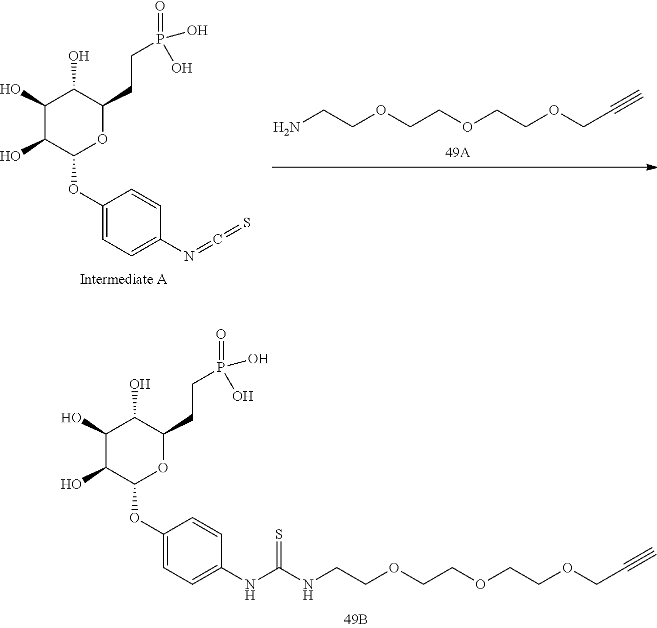 [SMILES: O=P(O)(O)CC[C@H]1O[C@H](Oc2ccc(N=C=S)cc2)[C@@H](O)[C@@H](O)[C@@H]1O ; C#CCOCCOCCOCCN ; C#CCOCCOCCOCCNC(=S)Nc1ccc(O[C@H]2O[C@H](CCP(=O)(O)O)[C@@H](O)[C@H](O)[C@@H]2O)cc1]

A solution of 2-(2-(2-(prop-2-yn-1-yloxy)ethoxy)ethoxy)ethan-1-amine (49A) (1.40 eq, 30.7 mg, 0.164 mmol) in NMP (0.6 mL) was added to Intermediate A (1.00 eq, 45.8 mg, 0.117 mmol) in a 1 dram vial with a stirbar. The resulting mixture was capped and stirred at room temperature for 18 h. Solids slowly dissolved to give a clear yellow solution. The reaction mixture was diluted with mixture of ethanol and acetic acid, filtered, and purified via preparatory HPLC (10-30% acetonitrile in water with 0.1% TFA). Fractions containing the desired product were combined. Most of the solvent was removed on a rotary evaporator at 29° C. and the remainder was lyophilized to dryness to afford Compound 49B as a white solid. Yield: 47.7 mg, 70%; LCMS m/z 579.4 [M+1]+; ¹H NMR (300 MHz, DMSO-d₆ with D₂O) δ 7.28 (d, J=8.6 Hz, 2H), 6.99 (d, J=8.5 Hz, 2H), 5.32 (s, 1H), 4.16-4.05 (m, 2H), 3.85-3.76 (m, 1H), 3.74-3.41 (m, 13H), 3.40-3.24 (m, 3H), 2.02-1.82 (m, 1H), 1.72-1.40 (m, 2H), 1.34-1.07 (m, 1H).

### Synthesis of Synthon 40A

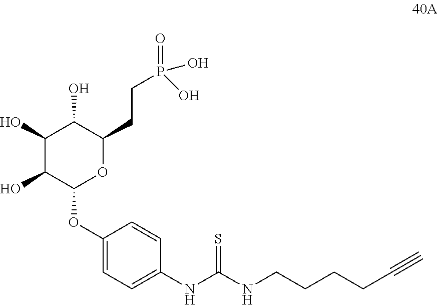 [SMILES: C#CCCCCNC(=S)Nc1ccc(O[C@H]2O[C@H](CCP(=O)(O)O)[C@@H](O)[C@H](O)[C@@H]2O)cc1]

40A is prepared from Intermediate A using similar methods as Synthon 49B.

### Synthesis of Synthon 59A

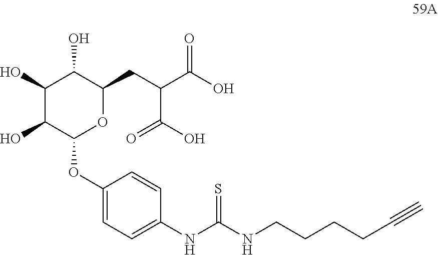 [SMILES: C#CCCCCNC(=S)Nc1ccc(O[C@H]2O[C@H](CC(C(=O)O)C(=O)O)[C@@H](O)[C@H](O)[C@@H]2O)cc1]

59A is prepared using similar methods as Synthon 49B.

### Synthesis of Synthon 60B

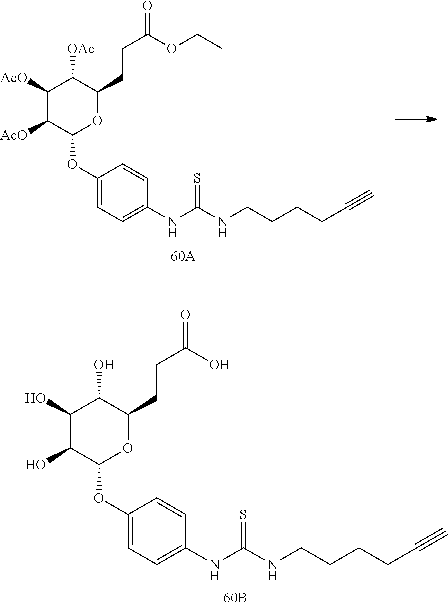 [SMILES: C#CCCCCNC(=S)Nc1ccc(O[C@H]2O[C@H](CCC(=O)OCC)[C@@H](OC(C)=O)[C@H](OC(C)=O)[C@@H]2OC(C)=O)cc1 ; C#CCCCCNC(=S)Nc1ccc(O[C@H]2O[C@H](CCC(=O)O)[C@@H](O)[C@H](O)[C@@H]2O)cc1]

To a round bottom flask was added (2R,3R,4S,5S,6R)-2-(3-ethoxy-3-oxopropyl)-6-(4-(3-(hex-5-yn-1-yl)thioureido)phenoxy)tetrahydro-2H-pyran-3,4,5-triyl triacetate (60A) (1.00 eq, 244 mg, 0.491 mmol) and THF (4 mL). To the stirring solution was added 3 M LiOH aq. (10.4 eq, 1.7 mL, 5.10 mmol). The reaction solution was allowed to stir at room temperature for 2 hrs. The reaction solution was diluted with EtOAc (30 mL) and aq. NH₄Cl. The organic phase was partitioned, washed with brine, dried over Na₂SO₄, filtered and concentrated in vacuo. The product Compound 60B (210 mg, 91% yield) was used with no additional purification. LC-MS m/z 453.6 [M+1]⁺.

### Synthesis of Synthon 46C

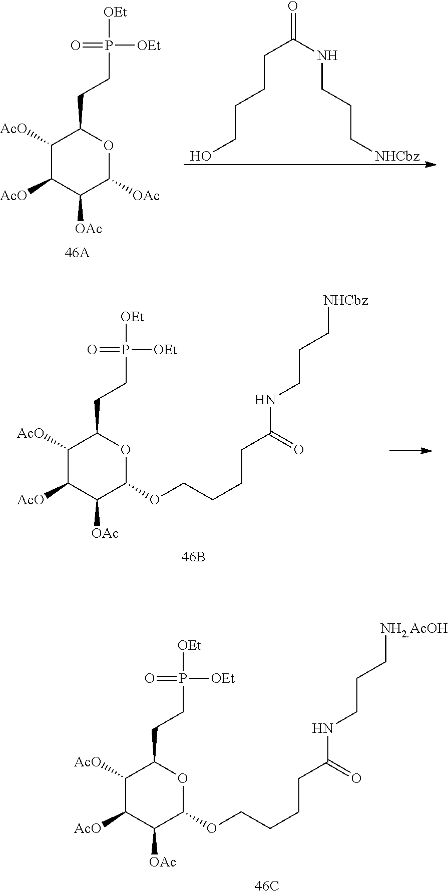

A solution of (2R,3S,4S,5R,6R)-6-(2-(diethoxyphosphoryl)ethyl)tetrahydro-2H-pyran-2,3,4,5-tetrayl tetraacetate (46A) (1.0 eq, 5.00 g, 10.4 mmol) and benzyl (3-(5-hydroxypentanamido)propyl)carbamate (2.0 eq, 6.39 g, 20.7 mmol) in DCM (100 mL) was cooled at 0° C., BF₃·Et₂O (12.0 eq, 15.4 mL, 124.0 mmol) was added dropwise and reaction mixture was heated at 50° C. for 16 h. Reaction was monitored by LCMS. After completion, reaction mixture was cooled at 0° C. and neutralized with triethylamine. Then, reaction mixture was diluted with DCM and washed with water. Organic layer was dried over anhydrous sodium sulfate, filtered and concentrated to get crude which was purified by reverse column chromatography using C-18 column and 20-50% acetonitrile in water to afford Compound 46B as a colorless viscous liquid. Yield: 3.10 g, 35.83%; LCMS m/z 731.29 [M+1]⁺.

To a solution of Compound 46B (1.0 eq, 2.6 g, 3.56 mmol) in methanol (26 mL), acetic acid (2.6 mL) and Palladium on carbon (10%) (1.3 g) was added and reaction mixture was stirred under hydrogen gas atmosphere at room temperature for 3 h. After completion, reaction mixture was filtered, filtrate was concentrated and dried to afford Compound 46C as colorless viscous liquid. Yield: 3.1 g (Crude); LCMS m/z 597.27 [M+1]⁺.

### Synthesis of Synthon 61A

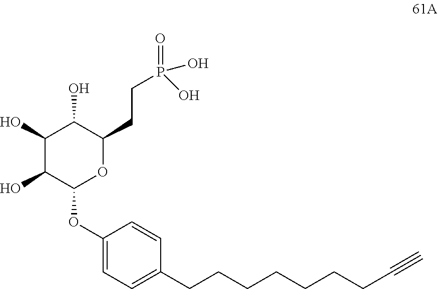 [SMILES: C#CCCCCCCCc1ccc(O[C@H]2O[C@H](CCP(=O)(O)O)[C@@H](O)[C@H](O)[C@@H]2O)cc1]

### Synthesis of Synthon 62A

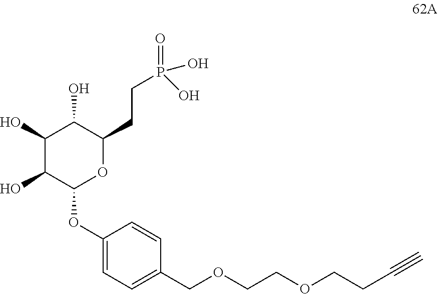 [SMILES: C#CCCOCCOCc1ccc(O[C@H]2O[C@H](CCP(=O)(O)O)[C@@H](O)[C@H](O)[C@@H]2O)cc1]

### Synthesis of Synthon 63A

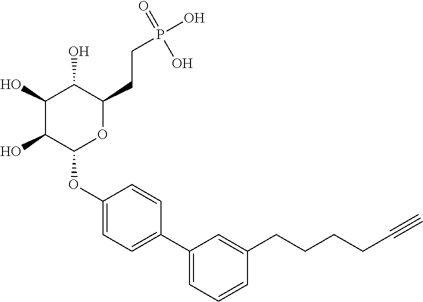 [SMILES: C#CCCCCc1cccc(-c2ccc(O[C@H]3O[C@H](CCP(=O)(O)O)[C@@H](O)[C@H](O)[C@@H]3O)cc2)c1]

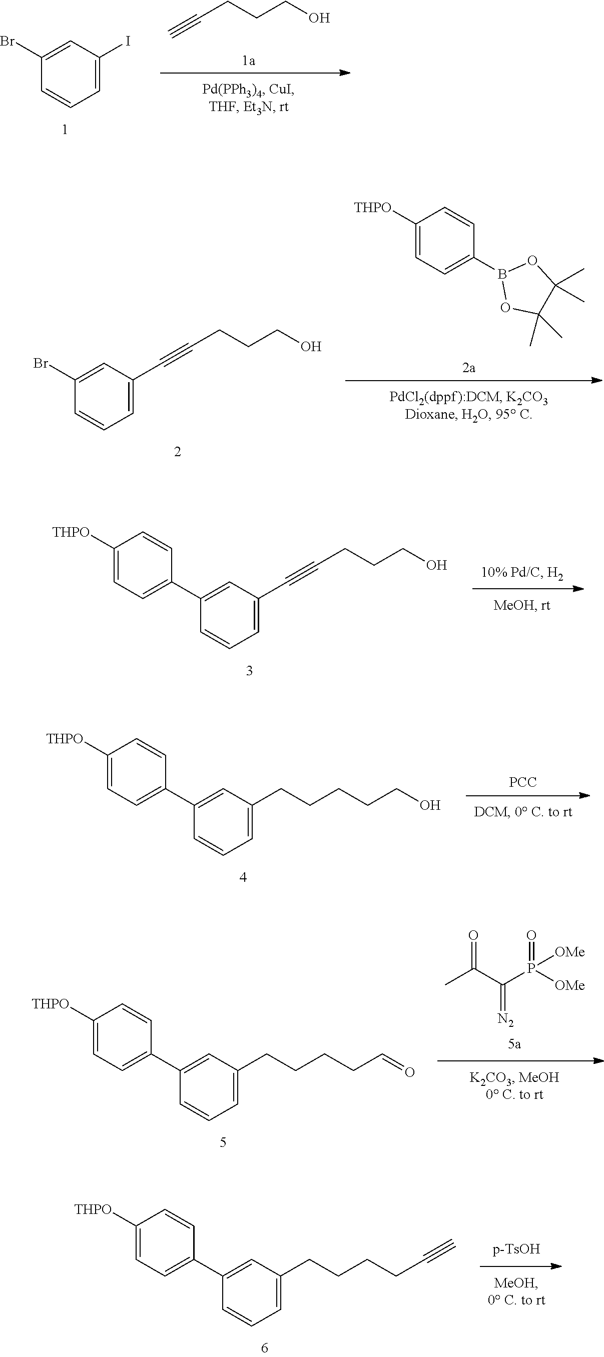

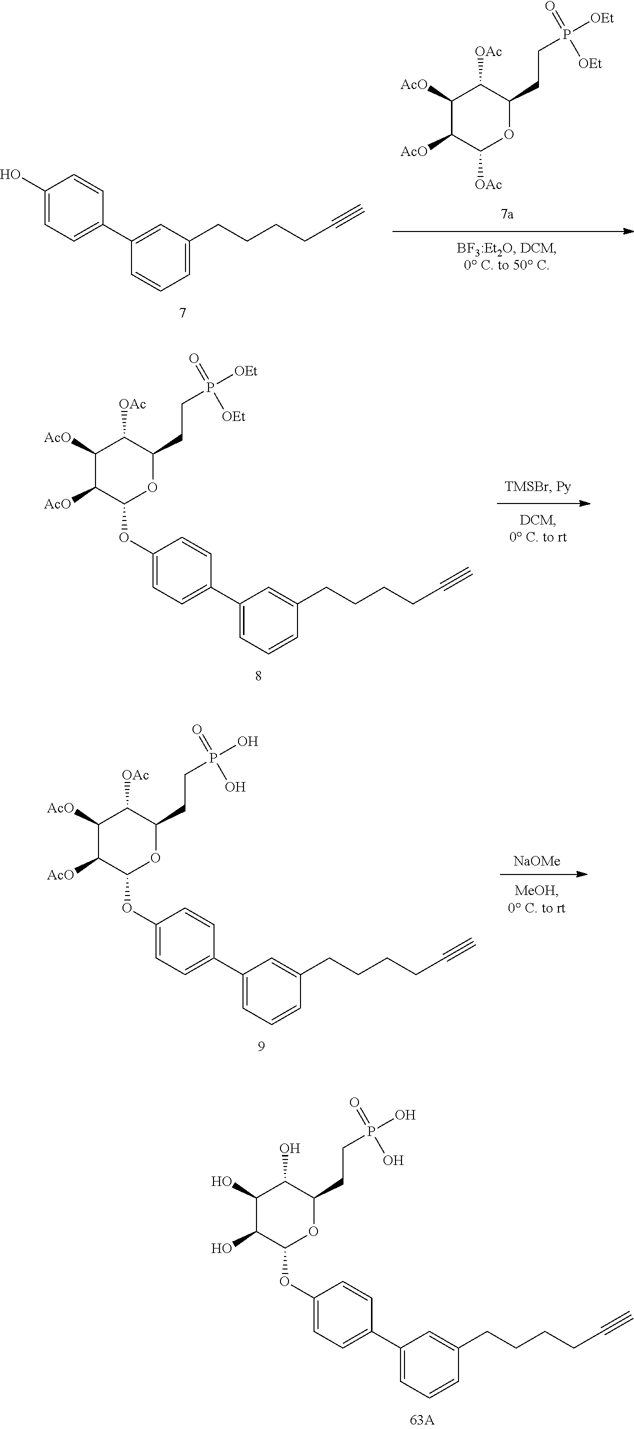 [SMILES: C#CCCCCc1cccc(-c2ccc(O)cc2)c1 ; CCOP(=O)(CC[C@H]1O[C@H](OC(C)=O)[C@@H](OC(C)=O)[C@@H](OC(C)=O)[C@@H]1OC(C)=O)OCC ; C#CCCCCc1cccc(-c2ccc(O[C@H]3O[C@H](CCP(=O)(OCC)OCC)[C@@H](OC(C)=O)[C@H](OC(C)=O)[C@@H]3OC(C)=O)cc2)c1 ; C#CCCCCc1cccc(-c2ccc(O[C@H]3O[C@H](CCP(=O)(O)O)[C@@H](OC(C)=O)[C@H](OC(C)=O)[C@@H]3OC(C)=O)cc2)c1 ; C#CCCCCc1cccc(-c2ccc(O[C@H]3O[C@H](CCP(=O)(O)O)[C@@H](O)[C@H](O)[C@@H]3O)cc2)c1 ; "NaOMe"]

Synthesis of 5-(3-bromophenyl)pent-4-yn-1-ol (2). To a solution of 1-bromo-3-iodobenzene (1, 16.8 g, 1.0 eq, 59.4 mmol) in tetrahydrofuran (90 mL) pent-4-yn-1-ol (1a, 5 g, 1.0 eq, 59.4 mmol), triethylamine (25.1 mL, 3.0 eq, 178 mmol) and copper(I) iodide (1.13 g, 0.1 eq, 5.94 mmol) were added and reaction mixture purged with flow of argon gas for 15 minutes. Tetrakis(triphenylphosphane) palladium (3.43 g, 0.05 eq, 2.97 mmol) was then added to reaction mixture and reaction mixture stirred at room temperature for 16 h. Reaction mixture partitioned in between ethyl acetate and water. Ethyl acetate layer separated, washed with water, brine, dried over anhydrous sodium sulphate, filtered and concentrated under reduced pressure to get crude product. crude product obtained was purified by flash column chromatography using silica gel column and eluting product in 10 to 30% ethyl acetate in hexane as eluents. Desired fractions were concentrated under reduced pressure to afford 5-(3-bromophenyl) pent-4-yn-1-ol (2) as brownish sticky gum. Yield: 14.0 g, 98.5%; LC-MS m/z 239.26 [M+1]⁺

Synthesis of 5-(4′-((tetrahydro-2H-pyran-2-yl)oxy)-[1,1′-biphenyl]-3-yl)pent-4-yn-1-ol (3). To a solution of 5-(3-bromophenyl)pent-4-yn-1-ol (2, 6.95 g, 1.3 eq, 29.1 mmol) in 1,4-dioxane (120 mL) was added 4,4,5,5-tetramethyl-2-[4-(oxan-2-yloxy)phenyl]-1,3,2-dioxaborolane (2a, 6.80 g, 1.0 eq, 22.4 mmol) and potassium carbonate solution (9.27 g, 3 eq, 67.2 mmol) in water (30.0 ml) and reaction mixture purged with argon gas for 15 minutes. [1,1′-Bis(diphenylphosphino)ferrocene]dichloropalladium(II):DCM (0.912 g, 0.05 eq., 1.12 mmol) was then added and reaction mixture stirred at 95° C. for 4 h. Reaction mixture quenched by addition of water and extracted with ethyl acetate. Ethyl acetate layer dried over anhydrous sodium sulphate and concentrated under reduced pressure to get crude product. Crude product obtained was purified by flash chromatography using silica gel column and eluting product in 10 to 30% Ethyl acetate in hexane as eluents. Desired fractions were concentrated under reduced pressure to afford (3) as colorless sticky gum. Yield: 4.90 g, 65.15%; LC-MS m/z 337.21 [M+1]⁺

Synthesis of 5-(4′-((tetrahydro-2H-pyran-2-yl)oxy)-[1,1′-biphenyl]-3-yl)pentan-1-ol (4). To a solution of (3) (0.25 g, 0.74 mmol) in methanol (10 mL) was added 10% palladium on carbon (0.080 g), Reaction mixture then stirred at room temperature under hydrogen atmosphere for 16 h. Reaction mixture filtered over celite pad, Filtrate obtained was concentrated under reduced pressure to afford (4) as colorless sticky gum. Yield: 0.24 g, 94.86%; LC-MS m/z 339.17 [M−1]⁻

Synthesis of 5-(4′-((tetrahydro-2H-pyran-2-yl)oxy)-[1,1′-biphenyl]-3-yl)pentanal (5). To a solution of (4) (0.470 g, 1.0 eq, 1.38 mmol) in dichloromethane (5 mL) at 0° C. was added pyridinium chloro chromate (0.446 g, 1.5 eq, 2.07 mmol) and reaction mixture stirred at room temperature for 4 h. After completion, reaction mixture was filtered over celite pad and washed with ether. Filtrate obtained was concentrated under reduced pressure and crude obtained was purified by combiflash chromatography using silica gel column and 10 to 20% ethyl acetate in hexane as eluents. Desired fractions were concentrated under reduced pressure to obtain (5) as colorless sticky gum. Yield: 0.290 g, 62.07%; LC-MS m/z 339.22 [M−1]⁻

Synthesis of 2-((3′-(hex-5-yn-1-yl)-[1,1′-biphenyl]-4-yl)oxy)tetrahydro-2H-pyran (6). To a solution of (5) (0.29 g, 1.0 eq, 0.857 mmol) in methanol (15.0 mL) was added potassium carbonate (0.296 g, 2.5 eq, 2.14 mmol) and 10% dimethyl (1-diazo-2-oxopropyl)phosphonate in acetonitrile (5a, 3.29 mL, 2.0 eq, 1.71 mmol) at 0° C. and reaction mixture was stirred at room temperature for 3 h. Reaction mixture quenched by addition of cold water and extracted with ethyl acetate. Ethyl acetate layer dried over anhydrous sodium sulphate and concentrated under reduced pressure to get crude compound. Crude compound obtained was purified by flash column chromatography using silica gel column and with 0 to 20% ethyl acetate in hexane as eluents. The desired fractions were concentrated under reduced pressure to get (6) as colorless sticky gum. Yield: 0.25 g, 87%; LC-MS m/z 353.25 [M+18]⁺

Synthesis of 3′-(hex-5-yn-1-yl)-[1,1′-biphenyl]-4-01 (7). To a solution of (6) (0.25 g, 0.747 mmol) in Methanol (3.00 mL) at 0° C., was added p-toluene sulphonic acid (0.014 g, 0.1 eq, 0.074 mmol) and reaction mixture stirred at room temperature for 2 h. Reaction mixture concentrated under reduced pressure and partitioned in between dichloromethane and aqueous sodium bicarbonate solution. Dichloromethane layer separated washed with brine solution, dried over anhydrous sodium sulphate and concentrated under reduced pressure to get crude product. Crude product obtained was purified by combiflash column chromatography using silica gel column and 5 to 15% Ethyl acetate in hexane as eluents. Desired fractions were concentrated under reduced pressure to afford (7) as colorless sticky gum. Yield: 0.16 g, 85%; LC-MS m/z 249.12 [M−1]⁻

Synthesis of (2R,3R,4S,5S,6R)-2-(2-(diethoxyphosphoryl)ethyl)-6-((3′-(hex-5-yn-1-yl)-[1,1′-biphenyl]-4-yl)oxy)tetrahydro-2H-pyran-3,4,5-triyl triacetate (8). To a stirred solution of (7a) (1.45 g, 1.5 eq., 3.00 mmol) and 3′-(hex-5-yn-1-yl)-[1,1′-biphenyl]-4-01 (7, 0.50 g, 1.0 eq, 2.00 mmol)) in dry dichloromethane (20 mL) was added activated molecular sieves (100 mg) and reaction mixture stirred at room temperature for 15 mins. Reaction mixture cooled to 0° C. and borontrifluoride etherate (1.48 mL, 6 eq, 12.0 mmol) was slowly added to reaction mixture and reaction mixture allowed to come at room temperature and stirred at 50° C. for 16 h. Reaction mixture partitioned in between dichloromethane and aqueous sodium bicarbonate solution. Dichloromethane layer separated and washed with brine solution, dried over anhydrous sodium sulfate, filtered and concentrated under reduced pressure to get crude product. Crude product obtained was purified by combiflash column chromatography using silica gel column and 30 to 50% Ethyl acetate in dichloromethane as eluents. Desired fractions were concentrated under reduced pressure to afford (8) as pale yellow sticky gum. Yield: 0.70 g, 52%; LC-MS m/z 673.39 [M+1]⁺

Synthesis of (2-((2R,3R,4S,5S,6R)-3,4,5-triacetoxy-6-((3′-(hex-5-yn-1-yl)-[1,1′-biphenyl]-4-yl)oxy)tetrahydro-2H-pyran-2-yl)ethyl)phosphonic acid (9). To the stirred solution of (8) (0.720 g, 1.0 eq, 1.07 mmol) in dichloromethane (30.00 mL) at 0° C., Pyridine (1.30 mL, 15 eq, 16.1 mmol) and bromotrimethylsilane (1.39 mL, 10 eq, 10.7 mmol) were added and reaction mixture was stirred at room temperature for 3 h. After completion reaction mixture was diluted with water and extracted with dichloromethane. Dichloromethane layer obtained was dried over anhydrous sodium sulphate and concentrated under reduced pressure to afford (9) as pale yellow sticky gum. Yield: 0.60 g, 90.92%; LC-MS m/z 615.11 [M−1]⁻

Synthesis of (2-((2R,3S,4S,5S,6R)-6-((3′-(hex-5-yn-1-yl)-[1,1′-biphenyl]-4-yl)oxy)-3,4,5-trihydroxytetrahydro-2H-pyran-2-yl)ethyl)phosphonic acid (63A). To a solution of (9) (0.630 g, 1 eq, 1.02 mmol) in Methanol (10.0 mL) at 0° C. was added Sodium methoxide solution (25%, 0.66 mL, 3 eq, 3.06 mmol) and reaction mixture stirred at room temperature for 3 h. LCMS showed formation of desired compound. Reaction mixture cooled down and neutralized Dowex 50W X8 hydrogen form up to pH 6 and filtered over sintered flask. Filtrate obtained was concentrated under reduced pressure to get crude product. Crude product obtained was purified by reverse phase preparative HPLC using 38-53% acetonitrile in water with 0.1% trifluoro acetic acid (0 to 10 minutes). Desired fractions were combined and lyophilized to afford (63A) as off white solid. Yield: 0.246 g, 49.09%; LC-MS m/z 491.13 [M+1]+H-NMR (400 MHz, DMSO-d6) δ 7.60 (d, J=8.8 Hz, 2H), 7.44-7.41 (m, 2H), 7.33 (t, J=7.60 Hz, 1H), 7.15-7.10 (m, 3H), 5.43 (s, 1H), 5.07-4.78 (bm, 3H), 3.84 (s, 1H), 3.67-3.65 (m, 1H), 3.38-3.28 (m, 2H), 2.74 (bs, 1H), 2.64 (t, J=7.20 Hz, 2H), 2.21-2.17 (m, 2H), 1.97-1.94 (m, 1H), 1.71-1.65 (m, 2H), 1.58-1.45 (m, 4H), 1.22-1.12 (m, 1H).

### Synthesis of Synthons 8 and 64A

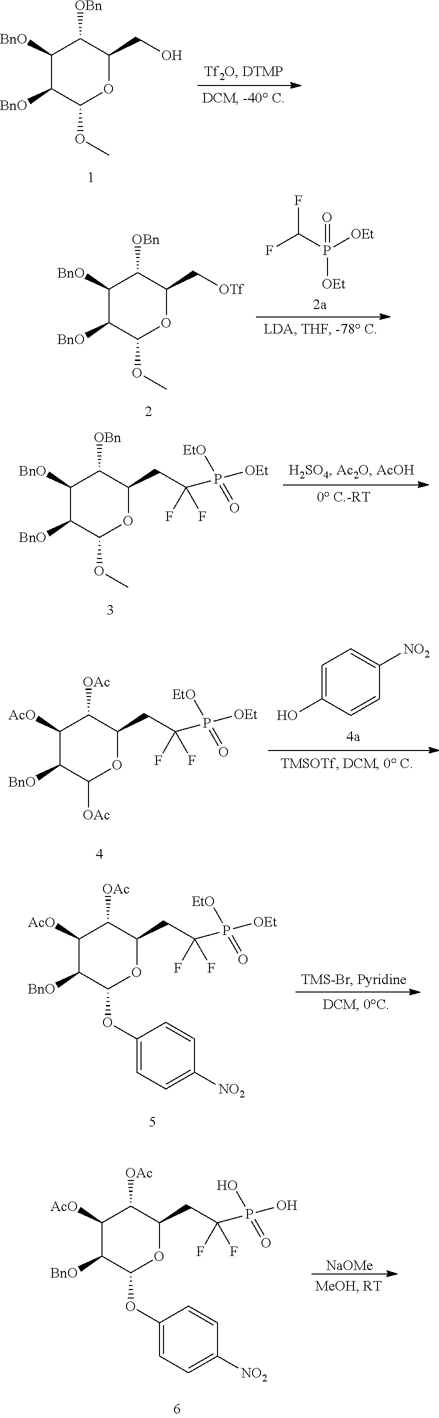

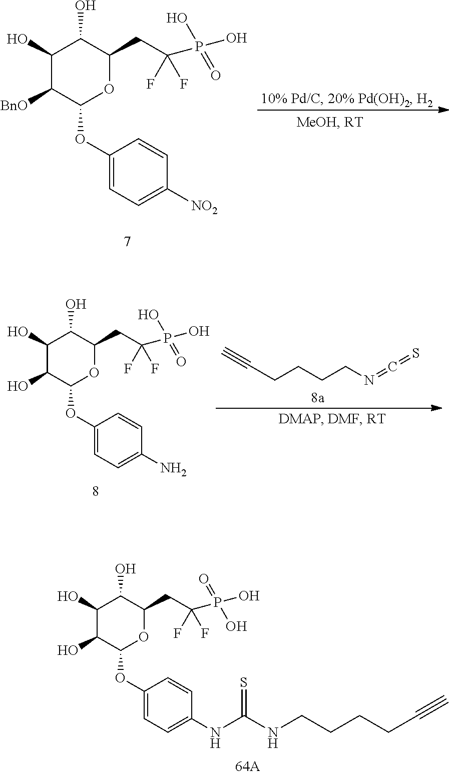

To the stirred solution of ((2R,3R,4S,5S,6S)-3,4,5-tris(benzyloxy)-6-methoxytetrahydro-2H-pyran-2-yl)methanol (1, 1.0 eq, 5.0 g, 10.8 mmol) in dichloromethane (50 mL), 2,6-di-tert-butyl-4-methylpyridine (1.8 eq, 3.32 g, 16.1 mmol) and trifluoromethanesulfonic anhydride (1.5 eq, 2.35 mL, 14.0 mmol) were added at −40° C. and reaction mixture was stirred at same temperature for 1 h. The progress of reaction was monitored by TLC. After completion, the reaction mixture was concentrated under reduced pressure to get crude product. The crude was immediately purified by flash column chromatography using 15-50% ethyl acetate in hexane to afford (2) as a pale yellow gel and immediately used for next reaction.

To a stirred solution of diethyl (difluoromethyl)phosphonate (2a, 4.0 eq, 5.30 g, 28.2 mmol) and [bis(dimethylamino)phosphoryl]dimethylamine (4.0 eq, 5.05 g, 28.2 mmol) in tetrahydrofuran (25 mL), Lithium di-isopropyl amide (LDA) 2 M in tetrahydrofuran (4.0 eq, 14.1 mL, 28.2 mmol) was added drop wise at −78° C. and stirred for 30 min at same temperature, Then a solution of (2, 1.0 eq, 4.20 g, 7.04 mmol) in tetrahydrofuran (25 mL) was added dropwise. The reaction mixture was stirred at −78° C. for 1 h. The progress of reaction was monitored by TLC. After completion, reaction mixture was quenched with saturated ammonium chloride solution, and extracted with ethyl acetate. The organic layer was dried over anhydrous sodium sulphate, filtered and concentrated under reduced pressure to get crude product. The crude product was purified by flash column chromatography using eluting from silica gel with 15-50% ethyl acetate in hexane to afford diethyl (1,1-difluoro-2-((2R,3R,4S,5S,6S)-3,4,5-tris(benzyloxy)-6-methoxytetrahydro-2H-pyran-2-yl)ethyl)phosphonate (3) as brown oil. Yield: 2.40 g, (49%) LCMS m/z 655.3 [M+18]⁺.

To a stirred solution of (3) (1.0 eq, 7.0 g, 11.0 mmol) in acetic anhydride (80.0 eq, 83.4 mL, 882 mmol) and acetic acid (132.0 eq, 83.3 mL, 1.46 mol). Sulfuric acid (6.5 eq, 3.82 mL, 71.7 mmol) was added at 0° C. and reaction mixture was stirred at room temperature for 16 h. The progress of reaction was monitored by TLC. After the completion, reaction mixture was concentrated under reduced pressure to get a residue. The residue was diluted with water and extracted with ethyl acetate. The organic layer was washed with saturated sodium bicarbonate solution, dried over anhydrous sodium sulphate, filtered and concentrated to get crude product. The crude was purified by flash column chromatography using 30-50% ethylacetate in hexane to afford (4) as colorless syrup. Yield: 3.20 g, (51%); LCMS m/z 566.3 [M+1]⁺.

To the stirred solution of (4, 1.0 eq, 3.20 g, 5.65 mmol) in dichloromethane (40 mL), 4-nitrophenol (4a, 3.0 eq, 2.36 g, 16.9 mmol) was added followed by trimethylsilyl trifluoromethanesulfonate (1.0 eq, 1.03 mL, 5.65 mmol) and reaction mixture was stirred at 0° C. for 4 h. The progress of reaction was monitored by TLC. After the completion of reaction, mixture was quenched with ice water and extracted with dichloromethane. The organic layer was dried over anhydrous sodium sulfate, filtered and concentrated to get crude. The crude was purified by flash column chromatography using 30-80% ethyl acetate in hexane to afford (5) as brown syrup. Yield: 2.45 g, (67.1%); LCMS m/z 663.20 [M+18]⁺.

To the stirred solution of (5, 1.0 eq, 1.00 g, 1.55 mmol) in dichloromethane (25 mL), pyridine (10.0 eq, 1.25 mL 15.5 mmol) followed by bromotrimethylsilane (10.0 eq, 2.0 mL, 15.5 mmol) was added at 0° C. and reaction mixture was stirred under for 16 h. The reaction mixture was monitored by LC-MS. After the completion of reaction, reaction mixture was quenched with ice water and extracted with dichloromethane. The organic layer was dried over anhydrous sodium sulfate, filtered and concentrated to get crude. The crude was triturated with diethyl ether and dried to get (6) acid as off white solid. Yield: 0.83 g, (90%); LCMS m/z 588.2 [M−1]⁻.

To a stirred solution of (6. 1.0 eq, 1.10 g, 1.87 mmol) dissolved in methanol (30 mL) and dichloromethane (10 mL) at 0° C., sodium methoxide 25% w/v in methanol (10.0 eq, 1.07 mL, 18.7 mmol) was added drop-wise. The reaction mixture was stirred at room temperature. After 3 h, the reaction mixture was neutralized with Dowex-50 hydrogen form (up to pH 7), filtered and filtrate was concentrated under reduced pressure to afford crude of (7) as off white solid Yield: 0.618 g, (66%); LCMS m/z 504.13 [M−1]⁻

To a stirred solution of (7, 1.0 eq, 0.55 g, 1.10 mmol) in methanol (10 mL), 10% Palladium on carbon (0.27 g) and 20% Pd(OH)₂ (0.27 g) were added and purged with hydrogen gas and stirred under hydrogen atmosphere for 5 h at room temperature. Then reaction mixture was filtered through a syringe filter (NY 0.45 μm). The filtrate was evaporated under reduced pressure to get crude of {2-[(2R,3S,4S,5S,6R)-6-(4-aminophenoxy)-3,4,5-trihydroxyoxan-2-yl]-1,1-difluoroethyl}phosphonic acid (8). The crude product was directly used for the next reaction without further purification. Yield: 0.31 g, (40.8%); LCMS m/z 386.1 [M+1]⁺

Synthesis of (1,1-difluoro-2-((2R,3S,4S,5S,6R)-6-(4-(3-(hex-5-yn-1-yl)thioureido)phenoxy)-3,4,5-trihydroxytetrahydro-2H-pyran-2-yl)ethyl)phosphonic acid (Cpd. No. 64A)

To a stirred solution of {2-[(2R,3S,4S,5S,6R)-6-(4-aminophenoxy)-3,4,5-trihydroxyoxan-2-yl]-1,1-difluoroethyl}phosphonic acid (8, 1.0 eq, 0.31 g, 0.815 mmol) and N,N-dimethylpyridin-4-amine (4.0 eq, 0.39 g, 3.26 mmol) in N,N-dimethylformamide (10 mL) at 0° C. was added a solution of 6-isothiocyanatohex-1-yne (8a, 3.0 eq, 0.34 g, 2.45 mmol) in N,N-dimethyl formamide (2 mL). The reaction mixture was then stirred at room temperature for 12 h. The reaction mixture was concentrated under reduced pressure to get crude. The crude was purified by prep-HPLC (10-30% Acetonitrile in water with 0.1% TFA). Fractions containing the desired product were combined and lyophilized to dryness to afford (64A) as white solid. Yield: 0.059 g, 13.8%; LCMS m/z 523.1[M−1]⁻.

### Synthesis of Synthons 7 and 65A

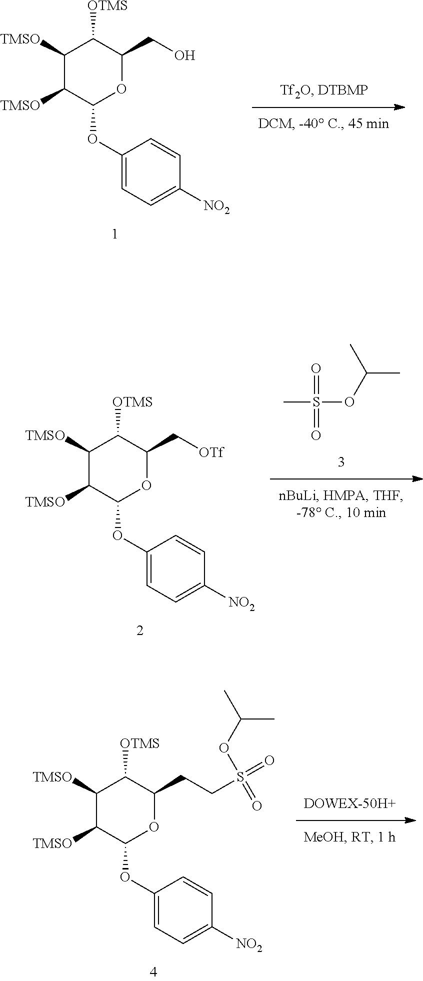

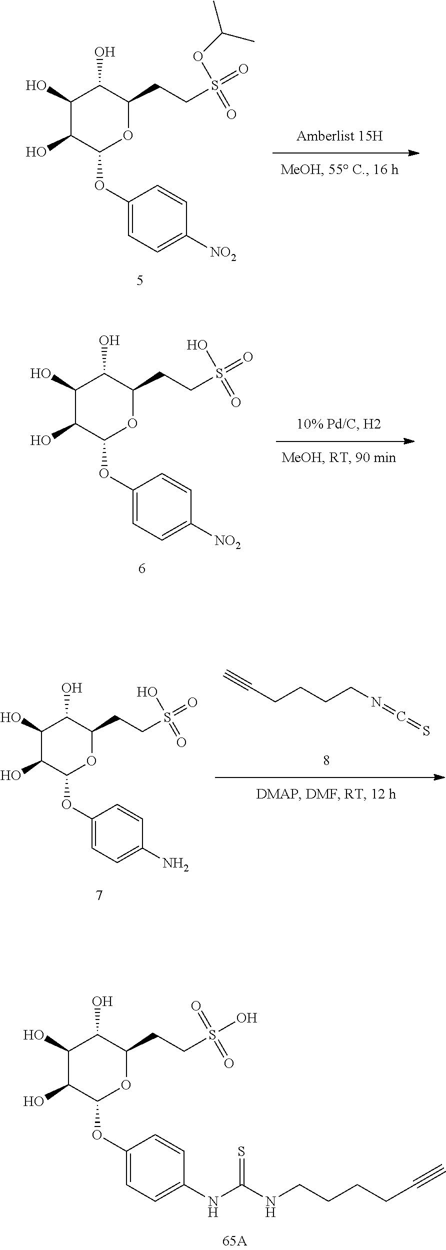

To a stirred solution of [(2R,3R,4S,5S,6R)-6-(4-nitrophenoxy)-3,4,5-tris[(trimethylsilyl)oxy]oxan-2-yl]methanol (1, 4.0 g, 7.73 mmol) and 2,6-di-tert-butyl-4-methylpyridine (3.17 g, 15.45 mmol) in dichloromethane (40.0 mL) was added trifluoromethanesulfonic anhydride (1.69 mL, 10.04 mmol) dropwise at −40° C. under a nitrogen atmosphere. After stirring for 1 h at −40° C., TLC showed full conversion. The volatiles were then evaporated and the crude (2) was directly used for the next reaction.

n-BuLi (12.3 mL, 30.8 mmol, 2.5 M solution in hexane) was added dropwise to a stirred solution of isopropyl methylsulfonate (3, 3.75 mL, 30.8 mmol) and [bis(dimethylamino)phosphoryl]dimethylamine (6.69 mL, 38.5 mmol) in dry tetrahydrofuran (60.0 mL) at −78° C. under nitrogen atmosphere. After 30 min, a pre-cooled solution of (2, 5.0 g, 7.69 mmol) in dry tetrahydrofuran (40.0 mL) was added to the reaction mixture. After 10 min, the reaction mixture was quenched with aq. ammonium chloride solution. The reaction mixture was extracted twice with ethyl acetate (50.0 mL) and washed with saturated aq. sodium bicarbonate solution. Organic fractions were collected and then dried over anhydrous sodium sulfate and filtered. The filtrate was evaporated under vacuum. The crude mass was purified by silica gel column chromatography (using 15% ethyl acetate in hexane) to afford (4) as yellowish solid. Yield: 2.4 g, 49%; LCMS m/z 655.3 [M+18]⁺.

To a stirred solution of (4, 1.7 g, 2.66 mmol) in methanol (80 mL) was added DOWEX-50H (10 g). After stirring for 1 h at room temperature, the resin was filtered off, washed with methanol, and the collected methanol portion was evaporated under vacuum. The crude reaction mass was then purified by silica gel column chromatography (using 10% methanol in dichloromethane), gave (5) as white foam. Yield: 0.845 g, 75%; LCMS m/z 420.1 [M−1]⁻

To a stirred solution of (5, 1.15 g, 2.73 mmol) in methanol (60 ml) was added Amberlist-15H (20 g) and heated at 55° C. for 16 h. The resin was then filtered off, washed with methanol, and the collected methanol portion was evaporated under vacuum. The crude product was purified by reverse phase column chromatography (eluting from a C18 column with 1-2% acetonitrile in water). The fractions containing the desired product were collected and lyophilized to provide (6) as white solid. Yield: 0.776 g, 75%; LCMS m/z 378.0 [M−1]⁻

To a stirred solution of (6, 0.103 g, 0.272 mmol) in methanol-water (10 ml, 9:1, v/v) was added 10% Pd/C (200.0 mg) and then purged with hydrogen gas and kept under hydrogen atmosphere for 90 min at room temperature. Then reaction mixture was filtered through NY 0.45 μm filter. The volatiles were then evaporated under reduced pressure to yield 2-((2R,3S,4S,5S,6R)-6-(4-aminophenoxy)-3,4,5-trihydroxytetrahydro-2H-pyran-2-yl)ethane-1-sulfonic acid (7) as white foam. Yield: 0.092 g, 96%; LCMS m/z 350.0 [M+1]⁺

To a stirred solution of (7, 0.179 g, 0.512.0 mmol) and N,N-dimethylpyridin-4-amine (0.188 g, 1.54 mmol) in N,N-dimethylformamide (10 mL) at 0° C. was added a solution of 6-isothiocyanatohex-1-yne (8, 0.214 mg, 1.54 mmol) in NN-dimethylformamide (2 mL). The reaction mixture was then stirred at room temperature for 12 h. After completion, reaction mixture was diluted with acetonitrile and purified by prep HPLC (15-47% acetonitrile in water with 0.1% TFA). Fractions containing the desired product were combined and lyophilized to dryness to afford Synthon 65A as a white solid. Yield: 0.080 g, 32%; LCMS m/z 489.2 [M+1]+; ¹H NMR (400 MHz, D₂O) 7.26-7.23 (m, 2H), 7.20-7.17 (m, 2H), 5.63 (s, 1H), 4.18 (s, 1H), 4.01 (d, J=9.6 Hz, 1H), 3.68. (t, J=9.6 Hz, 1H), 3.62-3.55 (m, 3H), 2.95-2.88 (m, 1H), 2.66-2.59 (m, 1H), 2.39-2.25 (m, 4H), 1.89-1.80 (m, 1H), 1.68 (brs, 2H), 1.53 (brs, 2H).

Synthesis of malonate synthon 66A

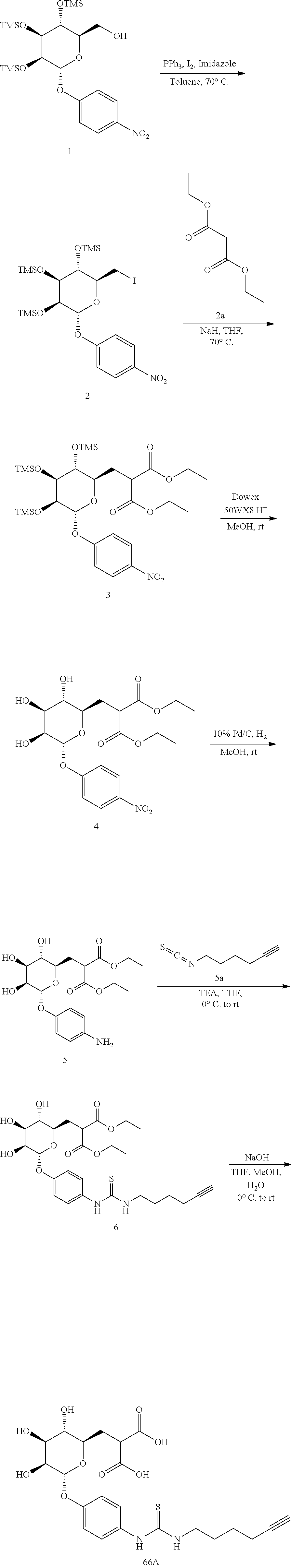

A solution of ((2R,3R,4S,5S,6R)-6-(4-nitrophenoxy)-3,4,5-tris((trimethylsilyl)oxy)tetrahydro-2H-pyran-2-yl)methanol (1, 1.00 g, 1.0 eq, 1.93 mmol), 1H-imidazole (0.394 g, 3 eq, 5.79 mmol), triphenyl phosphine (0.503 g, 1.0 eq, 1.93 mmol) and Iodine (0.61 g, 2.5 eq., 4.83 mmol) in toluene (15 mL), was heated to 70° C. and allowed to stir for another 12 h at this temperature. Reaction mixture was cooled down, diluted with ethyl acetate and quenched by addition of water. Ethyl acetate layer separated and aqueous layer re-extracted with ethyl acetate. Combined organic layer was dried over anhydrous sodium sulphate and evaporated under reduced pressure to get a crude residue which was purified by flash column chromatography using silica gel column and 0 to 3% ethyl acetate-hexane as eluents. Desired fractions were concentrated under reduced pressure to afford (2) as off white solid. Yield: 590 mg, 49%; LC-MS m/z 628.0 [M+1]⁺.

To a solution of diethylmalonate (1.99 g, 3 eq., 12.4 mmol) in dry tetrahydrofuran (20 mL) was added sodium hydride (0.497 g, 3 eq., 12.4 mmol) and stirred for 10 minutes. (2, 2.60 g, 1.0 eq, 4.14 mmol) in dry tetrahydrofuran (10 mL) was added slowly to reaction mixture and reaction mixture stirred at 70° C. for 24 h. TLC and LCMS showed presence of starting material and formation of desired product. Reaction mixture quenched by addition of cold water and extracted with ethyl acetate. Ethyl acetate layer dried over anhydrous sodium sulphate and concentrated under reduced pressure to get crude product. Crude product obtained was purified by combiflash using silica gel column (40 g) and a gradient of 3 to 10% ethyl acetate in hexane as eluents to recover starting material (2, 1.20 g) and afford the desired compound (3) as pale yellow sticky gum. Yield: 1.40 g, 51.2%; LC-MS m/z 658.2 [M−1]⁻.

To a solution of (3, 1.90 g, 1.0 eq, 2.88 mmol) in methanol (20.0 mL) was added Dowex 50W X8 hydrogen form (0.10 g) and reaction mixture stirred at room temperature for 3 h. Reaction mixture filtered over sintered glass funnel and filtrate obtained was concentrated under reduced pressure to get crude product. The crude product was purified by combiflash column chromatography using silica gel column (12 g) and 4 to 5% methanol in dichloromethane as eluents to afford (4) as pale yellow solid. Yield: 0.80 g, 62.6%; LC-MS 442.2 m/z [M−1]⁻.

To a solution of (4, 0.80 g, 1.0 eq, 1.80 mmol) in methanol (15 mL) was added 10% Pd/C (0.20 g) and reaction mixture stirred at room temperature under hydrogen atmosphere for 3 h. TLC showed consumption of starting material. The reaction mixture was filtered over a celite pad to remove catalyst and the filtrate was concentrated under reduced pressure to get pure (5) as pale yellow solid. Yield: 0.62 g, 83.1%; LC-MS m/z 414.1 [M+1]⁺.

To a solution of (5, 0.40 g, 1.0 eq, 0.968 mmol) in tetrahydrofuran (10.0 mL) at 0° C. was added triethylamine (0.337 mL, 2.5 eq, 2.42 mmol) and 6-isothiocyanatohex-1-yne (5a, 0.337 g, 2.5 eq, 2.42 mmol) dissolved in tetrahydrofuran (3 mL). Reaction mixture then stirred at room temperature for 16 h. Reaction mixture concentrated under reduced pressure and purified by combiflash column chromatography using silica gel column and eluting product in 5% methanol in dichloromethane as eluents. Desired fractions were concentrated under reduced pressure to afford (6) as pale yellow solid. Yield: 0.283 g, 50.2%; LC-MS m/z 553.3 (M+1)+

To a solution of (6, 0.28 g, 1.0 eq, 0.512 mmol) in tetrahydrofuran (10.0 mL) and methanol (1.0 mL) at 0° C. was added a solution of NaOH (0.041 g, 2 eq, 1.02 mmol) in water (0.5 mL) and reaction mixture stirred at room temperature for 1 h. LCMS showed formation of desired compound. Reaction mixture was neutralized with 2N hydrochloric acid to pH 6 and reaction mixture was concentrated under reduced pressure to get crude product. Crude product obtained was purified by reverse phase preparative HPLC (20 to 30% acetonitrile in water with 0.1% trifluoroacetic acid). Fractions containing the desired product were combined and lyophilized to dryness to afford 22-(((2R,3S,4S,5S,6R)-6-(4-(3-(hex-5-yn-1-yl)thioureido)phenoxy)-3,4,5-trihydroxytetrahydro-2H-pyran-2-yl)methyl)malonic acid (66A) as off white solid. Yield: 0.12 g, 47.9%; LC-MS m/z 497.2 (M+1)+

### Synthesis of Synthon Compound I-67

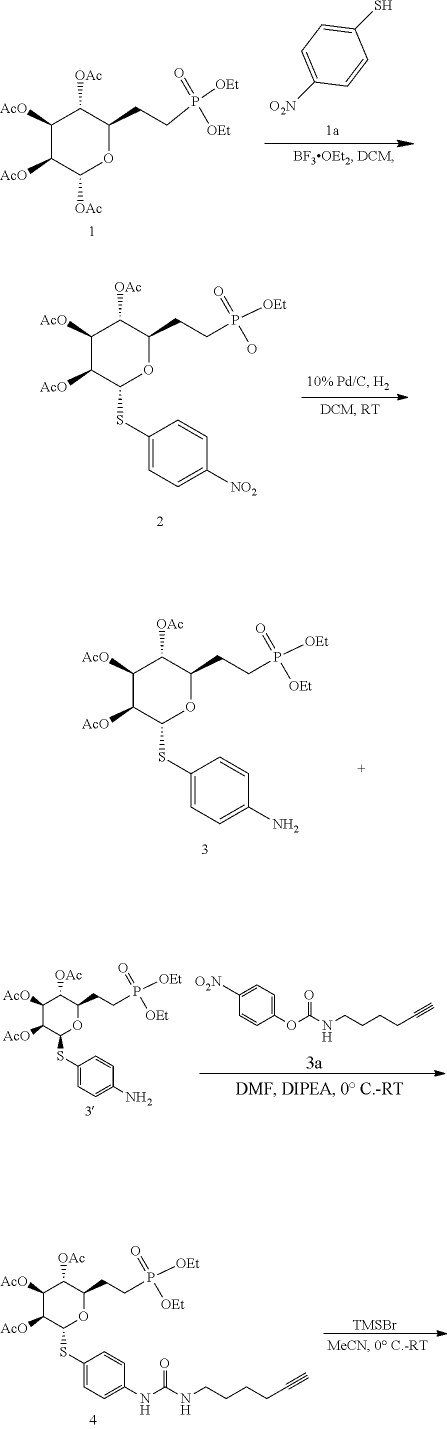

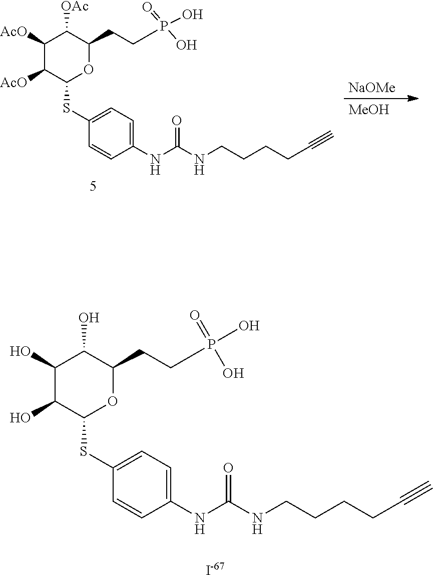

To a stirred solution of (2R,3S,4S,5R,6R)-6-(2-(diethoxyphosphoryl)ethyl)tetrahydro-2H-pyran-2,3,4,5-tetrayl tetraacetate (1, 1.0 eq, 6.0 g, 12.4 mmol) and 4-nitrothiophenol (5.0 eq, 9.65 g, 62.2 mmol) in dichloromethane (80 mL), was added boron trifluoride diethyl etherate (10.0 eq, 15.2 mL, 124 mmol) at 0° C. The reaction mixture was stirred at room temperature for 16 h. After that, reaction mixture was quenched with ice water, extracted with dichloromethane. The organic layer washed with saturated bicarbonate solution, followed by water and dried over anhydrous sodium sulfate, filtered and concentrated to get crude. The crude was purified by flash column chromatography using 50-100% ethyl acetate in hexane as eluent to afford a: isomer (7:3) (2) as a colorless sticky solid. Yield: 4.0 g, 55.7%; LC-MS, m/z. 578.14 [M+1]⁺.

To the stirred solution of (2, 1.0 eq, 1.2 g, 2.08 mmol) in dichloromethane (15.0 mL),10% Palladium on carbon (0.62 g, 50% w/w) were added and reaction mixture was stirred under hydrogen (balloon pressure) at room temperature for 16 h. The progress of reaction was monitored by LC-MS and TLC. After the completion of reaction, reaction mixture was filtered through syringe filter. The filtrate was concentrated under reduced pressure bath temperature <35° C.) to afford crude mixture of a: isomer (7:3) (2R,3S,4S,5R,6R)-2-((4-aminophenyl)thio)-6-(2-(diethoxyphosphoryl)ethyl)tetrahydro-2H-pyran-3,4,5-triyl triacetate (a isomer) and (2R,3S,4S,5R,6R)-2-((4-aminophenyl)thio)-6-(2-(diethoxyphosphoryl)ethyl)tetrahydro-2H-pyran-3,4,5-triyl triacetate (P isomer). The crude mixture was purified by prep-HPLC using (10-35% MeCN in water with 0.1% TFA). Fractions containing the desired product were combined and lyophilized to dryness to afford (2R,3S,4S,5R,6R)-2-((4-aminophenyl)thio)-6-(2-(diethoxyphosphoryl)ethyl)tetrahydro-2H-pyran-3,4,5-triyl triacetate (3) as off white solid. Yield: 0.65 g, 57%, a isomer; 0.2 g, 18%, p isomer LC-MS, m/z. 547.97 [M+1]⁺.

To a solution of (3, 1.0 eq, 0.65 g, 1.19 mmol) in NN-dimethyl formamide (5.0 mL) were added N,N-diisopropylethyl amine (1.0 eq, 0.20 mL, 1.19 mmol) and 4-nitrophenyl hex-5-yn-1-ylcarbamate (3a, 1.20 eq, 0.37 g, 1.42 mmol) solution in NN-dimethyl formamide (3.0 mL). The reaction mixture was stirred at room temperature for 16 h. The reaction mixture was then concentrated under reduced pressure to afford crude. The crude was purified by reverse phase (Aq C-18 column) column chromatography using 20-50% acetonitrile in water. The fractions were extracted with ethyl acetate and separated. The organic layer dried over anhydrous sodium sulfate, filtered and concentrated under reduced pressure to afford (4) as brown sticky solid. Yield: 0.33 g, 41.4%; LC-MS, m/z. 671.2 [M+1]⁺.

To a stirred solution of (4, 1.0 eq, 0.25 g, 0.373 mmol) in dichloromethane (8.0 mL), pyridine (10.0 eq, 0.30 mL, 3.73 mmol) and bromotrimethylsilane (10.0 eq, 0.49 mL, 3.73 mmol) was added at 0° C. and reaction mixture was stirred at room temperature for 16 h. After that, reaction mixture was quenched with ice water, extracted with dichloromethane. The organic layer dried over anhydrous sodium sulfate, filtered and concentrated under reduced pressure to get crude product. It was further washed with di-ethyl ether and dried to afford (5) as off white solid. Yield: 0.16 g, 69.84%; LC-MS, m/z. 614.93 [M+1]⁺.

To a stirred solution of (5) (1.0 eq, 0.16 g, 0.260 mmol) in methanol (5.0 mL), sodium methoxide 25% w/v in methanol (7.0 eq, 0.40 mL, 1.82 mmol) was added drop-wise and reaction mixture was stirred at room temperature for 2 h. After that, reaction mixture was neutralized with Dowex hydrogen form (200-400 mesh) to pH-7. The reaction mixture was then filtered, concentrated under reduced pressure to give crude product. The crude material was purified by prep-HPLC using (eluting from a C18 column with 50-80% MeCN in water with 0.1% TFA). Fractions containing the desired product were combined and lyophilized to dryness to afford (2-((2R,3S,4S,5S,6R)-6-((4-(3-(hex-5-yn-1-yl)ureido)phenyl)thio)-3,4,5-trihydroxytetrahydro-2H-pyran-2-yl)ethyl)phosphonic acid (I-67) as white solid. Yield: 0.058 g, 45.61%; LC-MS, m/z 488.9 [M+1]⁺. ¹H NMR (400 MHz, DMSO-d₆) δ 8.51 (s, 1H), 7.37 (d, J=8.8 Hz, 2H), 7.30 (d, J=8.8 Hz, 2H), 6.18 (t, J=5.6 Hz, 1H), 5.16 (s, 1H) 5.10 (brs, 1H), 4.79 (brs, 1H), 3.86 (s, 1H), 3.70 (t, J=7.2 Hz, 1H), 3.42 (dd, J=9.2, 3.2 Hz, 1H), 3.39-3.29 (m, 2H), 3.09-3.06 (m, 2H), 2.76 (t, J=2.8 Hz, 1H), 2.20-2.17 (m, 2H), 2.03-2.01 (m, 1H), 1.63-1.31 (m, 7H).

### Synthesis of Synthon Compound I-68

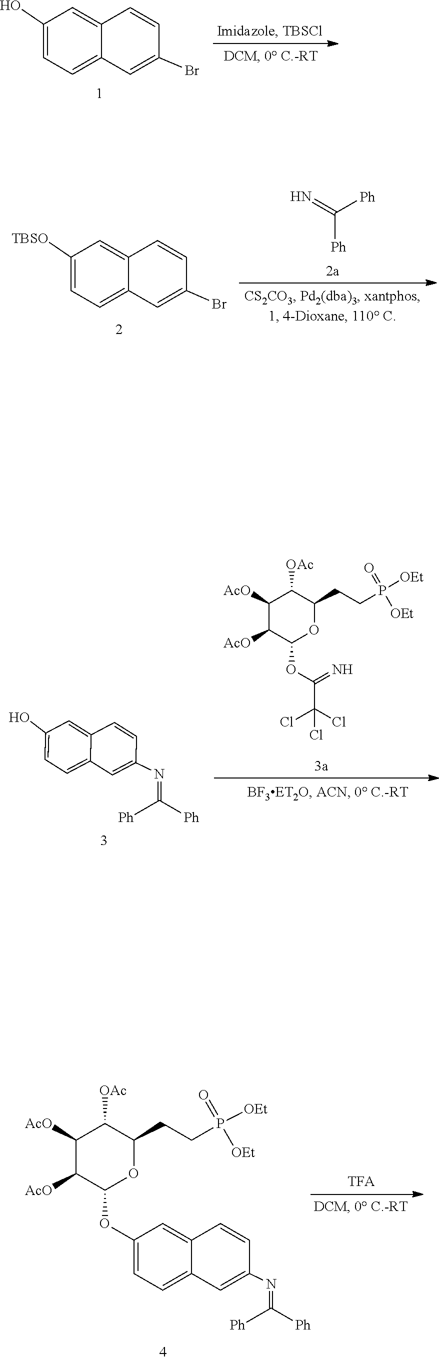 [SMILES: Oc1ccc2cc(Br)ccc2c1 ; CC(C)(C)[Si](C)(C)Oc1ccc2cc(Br)ccc2c1 ; N=C(c1ccccc1)c1ccccc1 ; Oc1ccc2cc(N=C(c3ccccc3)c3ccccc3)ccc2c1 ; CCOP(=O)(CC[C@H]1O[C@H](OC(=N)C(Cl)(Cl)Cl)[C@@H](OC(C)=O)[C@@H](OC(C)=O)[C@@H]1OC(C)=O)OCC ; CCOP(=O)(CC[C@H]1O[C@H](Oc2ccc3cc(N=C(c4ccccc4)c4ccccc4)ccc3c2)[C@@H](OC(C)=O)[C@@H](OC(C)=O)[C@@H]1OC(C)=O)OCC]

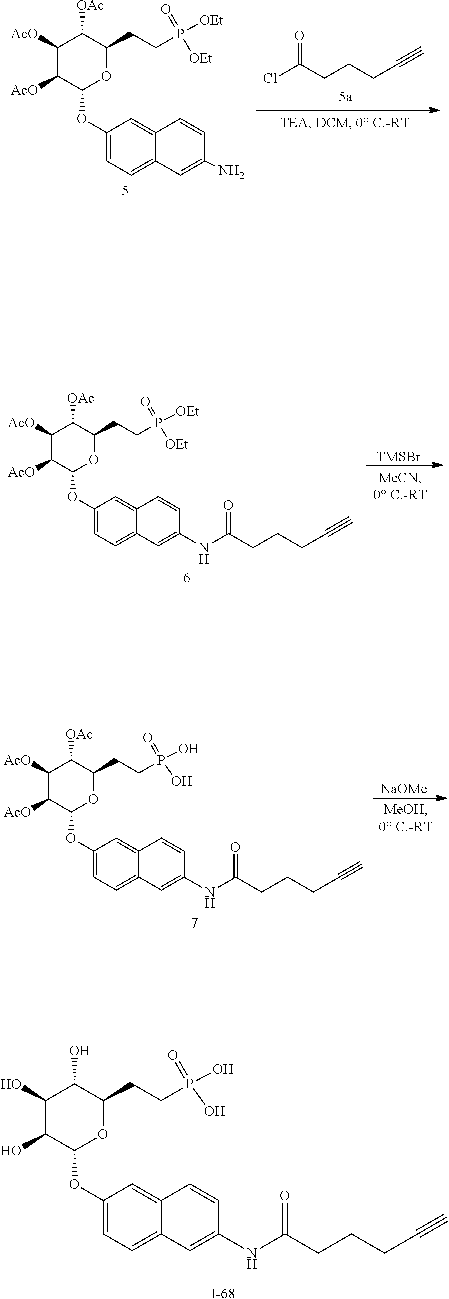

To a stirred solution of 6-bromonaphthalen-2-ol (1, 10.0 g, 1.0 eq., 44.8 mmol) in dichloromethane (50.0 mL), 1H-imidazole (6.1 g, 2.0 eq., 89.7 mmol) was added and the mixture was cooled to 0° C. tert-butyl(chloro)dimethylsilane (6.76 g, 1.0 eq., 44.8 mmol) was then added slowly. The reaction mixture was stirred at room temperature for 30 min and then diluted with dichloromethane and washed by water. Organic layer was separated, dried over anhydrous sodium sulfate, filtered and concentrated under reduced pressure to get crude which was purified by flash column chromatography using silica gel column (eluting with 5% ethyl acetate in hexane to afford (2) as an off white solid. Yield: 12.0 g, 79.3%; ¹H NMR (400 MHz, DMSO-d₆) δ 8.09 (s, 1H), 7.79 (q, J=9.6 Hz, 2H), 7.53 (dd, J=8.8, 1.6 Hz, 1H), 7.31 (d, J=1.6 Hz, 1H), 7.14 (dd, J=8.8, 2.4 Hz, 1H), 0.92 (s, 9H), 0.22 (s, 6H).

To stirred a solution of (2, 4.0 g, 1.0 eq., 11.9 mmol) in 1,4-dioxane (40.0 mL), diphenylmethanimine (2.15 g, 1.0 eq., 11.9 mmol) and cesium carbonate (5.41 g, 1.40 eq., 16.6 mmol) was added at room temperature. Argon gas was purged in reaction mixture for 10 min and then xantphos (0.685 g, 0.1 eq., 1.19 mmol) and tris(1,5-diphenylpenta-1,4-dien-3-one) dipalladium (0.543 g, 0.05 eq., 0.593 mmol) were added. The reaction mixture was then transferred to a pre-heated (at 110° C.) heating bath and stirred the reaction for 12 h. Water was added and extracted with ethyl acetate. The organic layer was separated, dried over sodium sulfate, filtered and concentrated with reduced pressure to get crude material. The crude product was purified by flash column chromatography using silica gel column (30-40% ethyl acetate in hexane) to afford (3) as a yellow colored solid. Yield: (0.80 g, 20.8%); LCMS, m/z 322.1 [M−1].

To a cold (−78° C.) stirred solution of (3a, 1.50 g, 1.0 eq, 2.57 mmol) and (3, 0.830 g, 2.57 mmol) in dichloromethane (10.0 mL) was added boron trifluoride diethyl etherate (0.633 mL, 2 eq., 5.13 mmol) at −78° C., and then the reaction mixture was stirred for 4 h at 0° C. After that, reaction mixture was diluted with dichloromethane and washed with water. Organic layer was separated, dried over anhydrous sodium sulfate and concentrated to get crude which was purified by flash column chromatography (30-40% ethyl acetate in dichloromethane) to afford (4), as yellow solid. Yield: 0.80 g, 42.0%; LC-MS, m/z 746.3 [M+1]⁺.

To a solution (4, 0.80 g, 1.0 eq., 1.07 mmol) in dichloromethane (15.0 mL), trifluoroacetic acid (3.00 mL) was added at 0° C., and reaction mixture was stirred for 6 h at room temperature. After that, reaction mixture was concentrated under reduced pressure to get the crude compound. The crude compound was purified by trituration with diethyl ether and pentane solvents to give (5) as a brown solid. Yield: 0.75 g, 60.0%, LC-MS, m/z-581.9, [M+1]⁺.

To a solution of (5, 0.80 g, 1.0 eq, 1.38 mmol) in dichloromethane (10.0 mL), triethylamine (0.580 mL, 3.0 eq., 4.13 mmol) and hex-5-ynoyl chloride (5a, 0.269 g, 1.50 eq., 2.06 mmol) were added at 0° C. and the reaction mixture was stirred for 4 h at room temperature. Water was added to the reaction mixture and extracted with dichloromethane. The combined organic fraction was dried over anhydrous sodium sulfate, filtered, and concentrated. The crude product was purified by flash column chromatography using silica gel column (using 3-4% methanol in dichloromethane) to afford (6) as a brown solid. Yield: 0.70 g, 45.0%; LC-MS, m/z 676.0 [M+1]⁺.

To a solution of (6, 0.70 g, 1.0 eq, 1.04 mmol) in dichloromethane (10.0 mL), pyridine (2.51 mL, 30 eq., 31.1 mmol) and bromotrimethylsilane (2.73 mL, 20 eq., 20.7 mmol) was added at 0° C. and the reaction mixture was stirred at room temperature for 3 h., After that, water was added and extracted with dichloromethane. The organic layer was dried over sodium sulfate, filtered and concentrated under reduced pressure to afford (7) as pale yellow sticky gum. yield: 0.50 g, 77.9%; LC-MS, m/z 618.2 [M−1].

To a solution of (7, 0.50 g, 0.807 mmol) in methanol (5.0 mL) was added 25% sodium methoxide solution (0.018 mL, 0.1 eq., 0.081 mmol) at 0° C. and the reaction mixture was stirred at room temperature for 1 h. After that, the reaction mixture was concentrated under reduced pressure to get crude compound which was purified by prep-HPLC (eluting from a C18 column with 30-40% acetonitrile in water with 0.1% TFA). The desired fractions were lyophilized to afford (2-((2R,3S,4S,5S,6R)-6-((6-(hex-5-ynamido)naphthalen-2-yl)oxy)-3,4,5-trihydroxytetrahydro-2H-pyran-2-yl)ethyl)phosphonic acid (I-68) as a white solid. Yield: (0.188 g, 47.2%) LC-MS, m/z 494.1 [M+1]⁺. ¹H NMR (400 MHz, DMSO-d₆) δ 10.06 (s, 1H), 8.23 (s, 1H), 7.76-7.72 (m, 2H), 7.52 (dd, J=8.8, 2.0 Hz, 1H), 7.42 (d, J=2.4 Hz, 1H), 7.20 (dd, J=9.2, 2.4 Hz, 1H), 5.51 (d, J=1.6 Hz, 1H), 3.88-3.87 (m, 1H), 3.68 (dd, J=8.4, 3.2 Hz, 1H), 3.39-3.34 (m, 4H), 2.83 (t, J=2.4 Hz, 1H), 2.46 (t, J=7.2 Hz, 2H), 2.24 (td, J=6.8, 2.4 Hz, 2H), 1.96-1.93 (m, 1H), 1.82-1.75 (m, 2H), 1.63-1.48 (m, 2H), 1.17-1.05 (m, 1H).

### Synthesis of Synthon Compound I-70

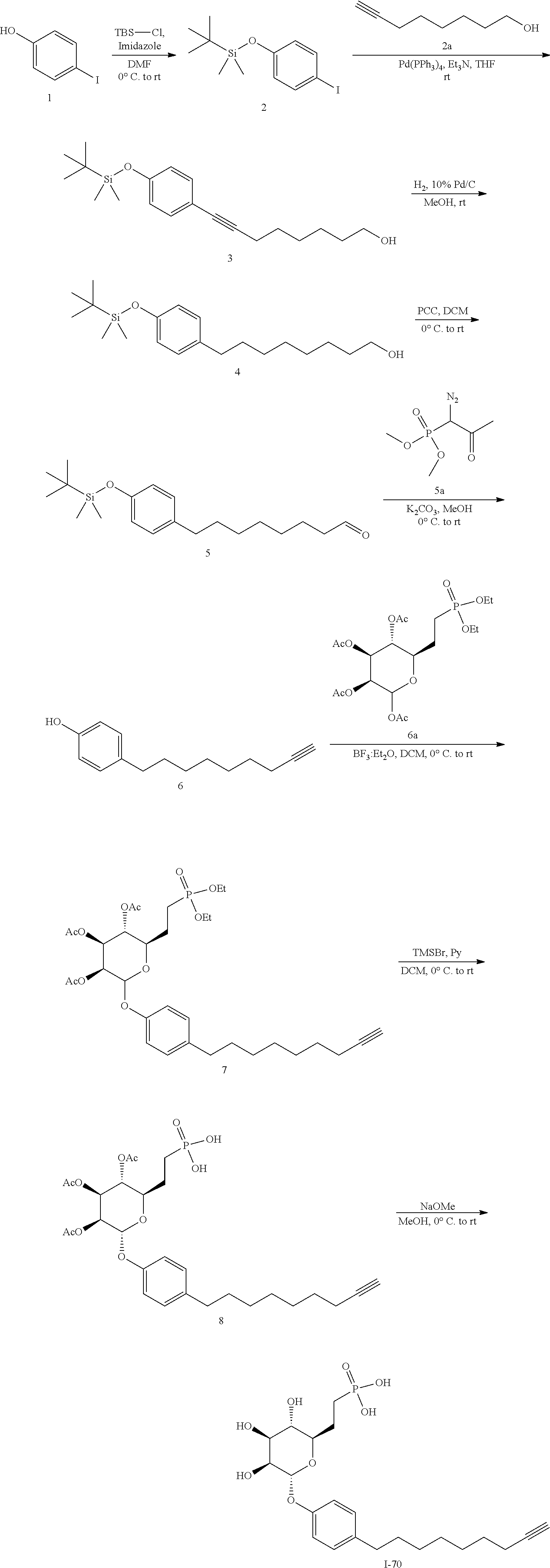

To the stirred solution of 4-iodophenol (1, 10 g, 1.0 eq, 45.5 mmol) and imidazole (7.74 g, 2.50 eq, 114 mmol) in Dimethylformamide (75.00 mL) at 0° C., tert-Butyldimethylsilyl chloride (10.3 g, 1.5 eq, 68.2 mmol) was added portion-wise and reaction mixture stirred at room temperature for 16 h. After completion, reaction was diluted with water and extracted with ethyl acetate. Organic layer was dried using anhydrous sodium sulfate, filtered and concentrated under reduced pressure to get crude residue which was purified flash column chromatography on silica gel column using 5 to 10% Ethyl acetate in hexane as eluents. Desired fractions were concentrated under reduced pressure to afford (2) as colorless oil. Yield: 14.0 g, 92.14%; ¹H NMR (400 MHz, CDCl₃) δ 7.49 (d, J=8.40 Hz, 2H), 6.60 (d, J=8.40 Hz, 2H), 0.96 (s, 9H), 0.18 (s, 6H).

To a solution of (2, 7.95 g, 1.0 eq, 23.8 mmol) in tetrahydrofuran (120.0 mL) was added oct-7-yn-1-ol (2a, 3.00 g, 1.0 eq, 23.8 mmol), triethyl amine (10.0 mL, 3.0 eq, 71.3 mmol) and copper(I) iodide (0.45 g, 0.1 eq, 2.38 mmol) and reaction mixture purged with flow of argon gas for 15 minutes. tetrakis(triphenylphosphane) palladium (1.37 g, 0.05 eq, 1.19 mmol) was added to reaction mixture and reaction mixture stirred at room temperature for 16 h. Reaction mixture partitioned in between ethyl acetate and water. Ethyl acetate layer separated and washed with water, brine, dried over anhydrous sodium sulphate and concentrated under reduced pressure to get crude product. crude product obtained was purified by flash column chromatography on silica gel column eluting product in 10 to 30% ethyl acetate in hexane as eluents. Desired fractions were concentrated under reduced pressure to afford (3) brown color sticky gum. Yield: 5.20 g, 65.78%; LCMS m/z 333.30 [M+1]⁺

To a solution of (3, 4.00 g, 1.0 eq, 12.0 mmol) in Methanol (30 mL) was added 10% palladium on carbon (0.400 g), Reaction mixture then stirred under hydrogen atmosphere at room temperature for 16 h. Completion of reaction was monitored by LCMS. The reaction mixture filtered over celite pad, filtrate obtained was concentrated under reduced pressure to afford (4) as colorless sticky gum. Yield: 3.90 g, 96%; ¹H NMR (400 MHz, CDCl₃) δ 7.00 (d, J=8.00 Hz, 2H), 6.73 (d, J=8.40 Hz, 2H), 3.65-3.58 (m, 2H), 2.51 (d, J=8.00 Hz, 2H), 1.55 (bs, 2H), 1.47 (bs, 2H),1.31 (bs, 9H), 0.97 (s, 9H), 0.18 (s, 6H).

To a solution of (4, 3.90 g, 1.0 eq, 11.6 mmol) in Dichloromethane (100 mL) at 0° C. was added Pyridinium chloro chromate (3.25 g, 1.3 eq, 15.1 mmol) and reaction mixture stirred at room temperature for 4 h. TLC showed formation of product. Reaction mixture filtered over celite pad and washed with ether. Filtrate concentrated under reduced pressure and crude obtained was column purified eluting compound in hexane to 5% ethyl acetate in hexane as eluents. Desired fractions were concentrated under reduced pressure to obtain (5) as colorless oil. Yield: 2.60 g, 57.90%; LCMS m/z 335.35 [M+1]⁺

To a solution of (5, 0.65 g, 1.0 eq, 1.94 mmol) in methanol (20.0 mL) at 0° C. was added potassium carbonate (0.805 g, 3 eq., 5.83 mmol) and 10% dimethyl (1-diazo-2-oxopropyl)phosphonate in acetonitrile (5a, 7.46 mL, 2 eq, 3.89 mmol) and reaction mixture stirred at room temperature for 4 h. Reaction mixture quenched by addition of cold water and extracted with ethyl acetate. Ethyl acetate layer dried over anhydrous sodium sulphate and concentrated under reduced pressure to get crude compound. Crude compound obtained was purified by flash column chromatography using silica gel column eluting compound in 5 to 20% Ethyl acetate in hexane. The desired fractions were concentrated under reduced pressure to get 4-(non-8-yn-1-yl) phenol (6) as colorless sticky gum. Yield: 0.350 g, 83.28%; LCMS m/z 215.19[M−1]⁻

To a stirred solution of 4-(non-8-yn-1-yl)phenol (6, 0.30 g, 1.0 eq, 1.39 mmol) and (3S,4S,5R,6R)-6-(2-(diethoxyphosphoryl)ethyl)tetrahydro-2H-pyran-2,3,4,5-tetrayl tetraacetate (6a, 0.669 g, 1.0 eq, 1.39 mmol) in Dichloromethane (8.0 mL) was added activated molecular sieves (0.100 g) and reaction mixture stirred at room temperature for 15 mins. Reaction mixture cooled to 0° C. and borontrifluoride etherate (1.03 mL, 6 eq, 8.32 mmol) was added to reaction mixture and stirred at room temperature for 16 h. Reaction mixture cooled down and partitioned in between dichloromethane and aqueous sodium bicarbonate solution. Dichloromethane layer separated and washed with brine solution, dried over anhydrous sodium sulphate and concentrated under reduced pressure to get crude product. Crude product obtained was purified by combiflash column chromatography using silica gel column and eluting product in 30 to 50% Ethyl acetate in dichloromethane as eluents to afford (7) as colorless sticky gum. Yield: 0.35 g, 33.87%; LCMS m/z 639.49 [M+1]⁺

To the stirred solution of (7, 0.35 g, 1.0 eq, 0.546 mmol) in dichloromethane (7.00 mL) at 0° C., Pyridine (0.663 mL, 15 eq., 8.22 mmol) and Bromotrimethylsilane (0.711 mL, 10 eq, 5.48 mmol) were added and reaction mixture was stirred at room temperature for 3 h and reaction was monitored by LCMS. After completion reaction mixture was diluted with water and concentrated under reduced pressure to get crude product. Crude product obtained was diluted with diethyl ether and filtered. Filtrate was concentrated under reduced pressure to afford (8) as pale yellow sticky gum. yield: 0.25 g, 78%; LCMS m/z 581.35 [M−1]⁻

To a solution of (8, 0.25 g, 1.0 eq, 0.429 mmol) in Methanol (4.0 mL) at 0° C. was added Sodium methoxide solution (25%, 3 eq, 0.27 mL, 1.28 mmol) and reaction mixture stirred at room temperature for 3 h. LCMS showed formation of desired compound. Reaction mixture cooled down and neutralized with Dowex 50WX8 hydrogen form and filtered over sintered flask. Filtrate obtained was concentrated under reduced pressure to get crude product. Crude product obtained was purified by preparative HPLC (30-62% acetonitrile in water with 0.1% TFA) to afford (2-((2R,3S,4S,5S,6R)-3,4,5-trihydroxy-6-(4-(non-8-yn-1-yl)phenoxy)tetrahydro-2H-pyran-2-yl)ethyl)phosphonic acid (I-70) as off white solid. Yield: 0.075 g, 38.29%, LCMS m/z 457.31 [M+1]¹H NMR (400 MHz, DMSO-d6) δ 7.08 (d, J=8.0 Hz, 2H), 6.92 (d, J=8.4 Hz, 2H), 5.00-4.74 (m, 3H), 3.79 (s, 1H), 3.63-3.60 (m, 1H), 3.39-3.28 (m, 6H), 2.72 (t, J=2.4 Hz, 2H), 2.14-2.10 (m, 2H), 1.91 (bs, 1H), 1.62-1.51 (m, 4H), 1.43-1.40 (m, 2H), 1.30-1.17 (m, 7H).

### Synthesis of Synthon Compound I-71

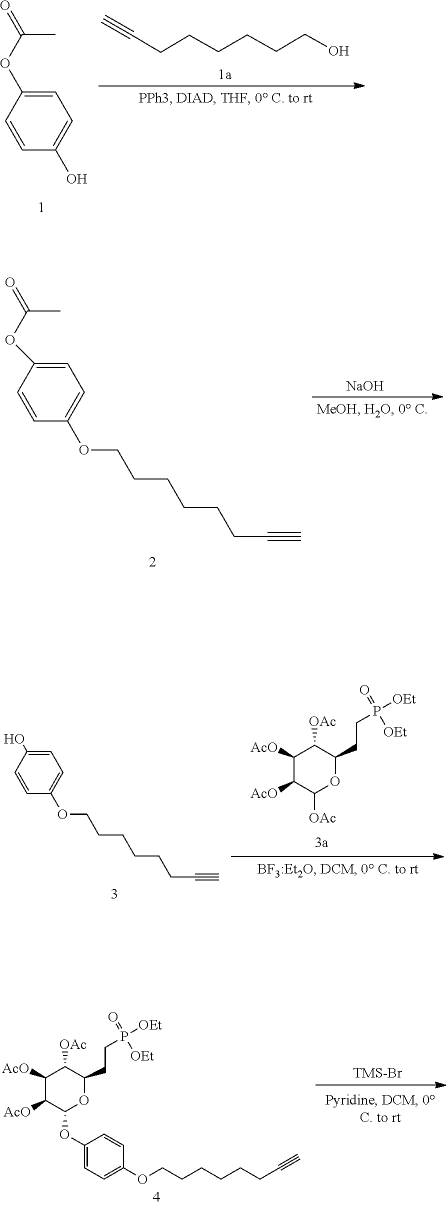

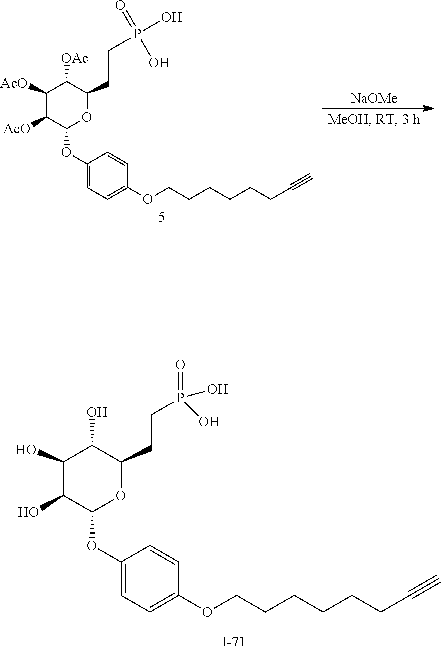

To the stirred solution of 4-hydroxyphenyl acetate (1, 5.00 g, 1.0 eq, 0.032 mol) and oct-7-yn-1-ol (1a, 4.14 g, 1.0 eq, 0.032 mol) in tetrahydrofuran (50 mL) at 0° C., triphenyl phosphine (9.22 g, 1.1 eq, 0.035 mol) and diisopropyl azodicarboxylate (7.11 g, 1.1 eq, 0.035 mol) were added and reaction mixture stirred for 16 h at room temperature. After completion reaction mixture was diluted with water and extracted with ethyl acetate. Ethyl acetate layer was dried over anhydrous sodium sulfate and concentrated to get crude compound. The crude compound was purified by combi flash column chromatography using silica gel column and 5 to 7% ethyl acetate in hexane as eluents. Desired fractions were concentrated under reduced pressure to afford 4-(oct-7-yn-1-yloxy)phenyl acetate (2) as colorless liquid. Yield: 6.0 g, 70.13%; LC-MS m/z 259.18 [M−1]⁻.

To the stirred solution of 4-(oct-7-yn-1-yloxy)phenyl acetate (2, 6.0 g, 1.0 eq, 0.023 mol) in methanol (36.0 mL) at 0° C., sodium hydroxide (1.84 g, 2.0 eq, 0.046 mol) dissolved in water (24.0 mL), was added and reaction mixture was stirred at same temperature for 30 min. After completion reaction mixture was concentrated under reduced pressure and then diluted with water and compound was extracted with ethyl acetate. Ethyl acetate layer was dried over anhydrous sodium sulfate and concentrated under reduced pressure to afford 4-(oct-7-yn-1-yloxy)phenol (3) as off white solid. Yield: 5.0 g, 99.38%; LC-MS m/z 217.14 [M−1]⁻.

To a stirred solution of 4-(oct-7-yn-1-yloxy)phenol (3, 0.905 g, 3.0 eq, 4.15 mmol) and (2R,3S,4S,5R,6R)-6-(2-(diethoxyphosphoryl)ethyl)tetrahydro-2H-pyran-2,3,4,5-tetrayl tetraacetate (3a, 1.0 g, 1.0 eq, 1.39 mmol) in Dichloromethane (10.0 mL) was added activated molecular sieves (0.10 g) and reaction mixture stirred at room temperature for 15 mins. Reaction mixture cooled to 0° C. and borontrifluoride etherate (2.76 mL, 6 eq, 12.4 mmol) was added to reaction mixture and reaction mixture stirred at room temperature for 6 h. Reaction mixture cooled down and partitioned in between dichloromethane and aqueous sodium bicarbonate solution. Dichloromethane layer separated and washed with brine solution, dried over anhydrous sodium sulphate and concentrated under reduced pressure to get crude product. Crude product obtained was purified by combiflash column chromatography eluting product in 50-60% Ethyl acetate in hexane as eluents to afford (4) as off white solid. Yield: 0.60 g, 45.18%; LC-MS m/z 641.26 [M+1]⁺.

To the stirred solution of (4, 0.600 g, 1.0 eq, 0.937 mmol) in dichloromethane (10.0 mL) at 0° C. Pyridine (0.741 ml, 10.0 eq, 9.37 mmol) was added and stirred for 5 min. Bromotrimethylsilane (1.24 ml, 10.0 eq, 9.37 mmol) was added dropwise in reaction mixture. Reaction was stirred at room temperature for 3 h and reaction was monitored by LCMS. Reaction mixture was diluted with water and dichloromethane. Dichloromethane layer separated and aqueous layer re-extracted dichloromethane. Combined dichloromethane was dried over anhydrous sodium sulfate and concentrated under reduced pressure to afford (5) as yellow liquid. Yield 0.500 g, 84.31%; LC-MS m/z 583.44 [M−1]⁻

To the solution of (5, 0.50 g, 1.0 eq, 0.856 mmol) in methanol (6.00 mL) at 0° C., Sodium methoxide solution (0.94 mL, 5.0 eq, 4.280 mmol) was added drop wise and reaction mixture stirred at room temperature for 3 h. After completion reaction was quenched with Dowex 50WX8 hydrogen form and filtered on sintered funnel. Filtrate obtained was concentrated under reduced pressure to get crude compound. The crude compound was purified by reverse phase preparative HPLC (37-57% acetonitrile in water with 0.1% TFA) to afford (2-((2R,3S,4S,5S,6R)-3,4,5-trihydroxy-6-(4-(oct-7-yn-1-yloxy)phenoxy)tetrahydro-2H-pyran-2-yl)ethyl)phosphonic acid (I-71) as off white solid. Yield: 0.202 g, 51.51%; LCMS m/z 459.27 [M+1]+, H-NMR (400 MHz, DMSO-d6) δ 6.94 (d, J=9.2 Hz, 2H), 6.88 (d, J=9.2 Hz, 2H), 5.23 (d, J=1.2 Hz, 1H), 4.98 (bs, 1H), 4.72 (bs, 1H), 3.88 (t, J=6.4 Hz, 2H), 3.79 (s, 1H), 3.60 (d, J=4.8 Hz, 1H), 3.34-3.30 (m, 2H), 2.73 (t, J=2.4 Hz, 1H), 2.17-2.13 (m, 2H), 1.96-1.93 (m, 1H), 1.66 (t, J=6.4 Hz, 2H), 1.62-1.40 (m, 9H), 1.23-1.12 (m, 1H).

### Synthesis of Synthon Compound I-73

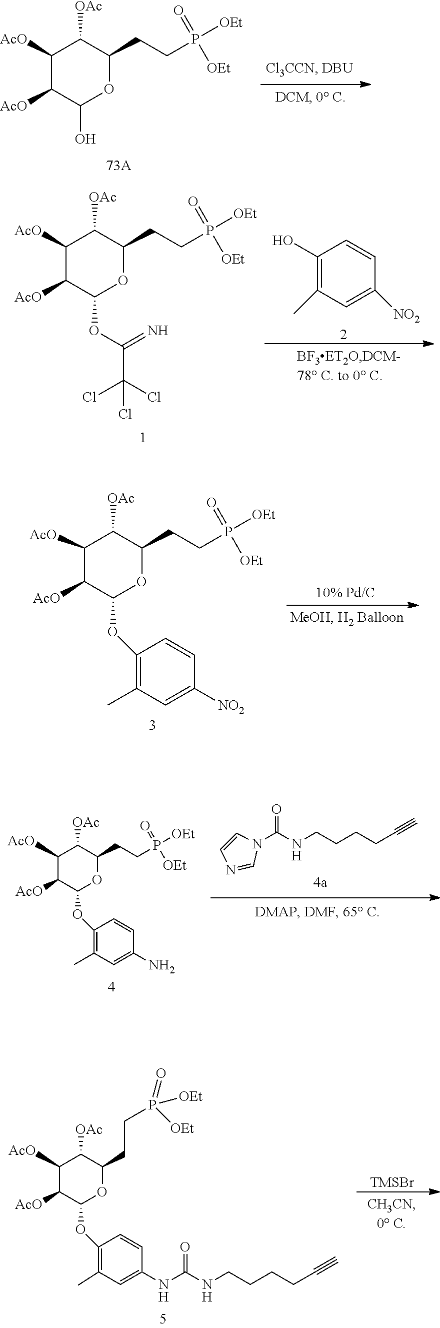

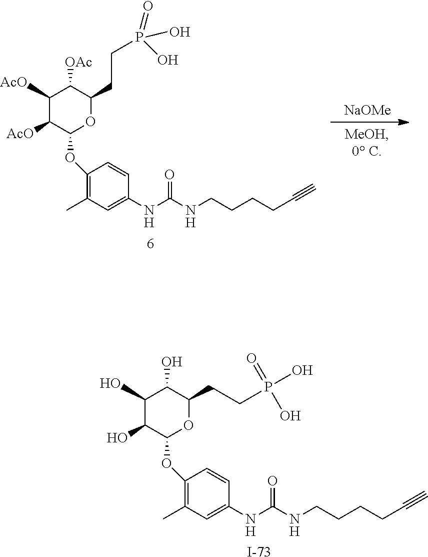

1,8-Diazabicyclo[5.4.0]undec-7-ene (0.085 mL, 0.568 mmol) was added to a stirred solution of [(2R,3R,4S,5S,6S)-4,5-diacetoxy-2-(2-diethoxyphosphorylethyl)-6-hydroxy-tetrahydropyran-3-yl]acetate (73A, 2.5 g, 5.68 mmol) and trichloroacetonitrile (5.69 mL, 56.8 mmol) in dichloromethane (30.0 mL) at 0° C. under nitrogen. The resulting mixture was stirred at 0° C. under nitrogen. TLC at 30 min (100% ethyl acetate) shows conversion to less polar spot. Most of the solvent was removed on a rotary evaporator. The residue was loaded onto a silica gel loading column which was pre-equilibrated with 0.1% triethylamine in dichloromethane and purified via silica gel chromatography (column pre-equilibrated with 0.1% triethylamine in 20% ethyl acetate/dichloromethane) (20-100% ethyl acetate in dichloromethane) to afford (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (1) as a colorless semi-solid compound. Yield: 2.8 g, 84.35%.

(1, 2.8 g, 4.79 mmol) was dissolved in dry dichloromethane (25 mL) with stirring under nitrogen. 2-methyl-4-nitrophenol (2, 1.83 g, 12.0 mmol) was added and the resulting clear solution was cooled to −78° C. with stirring under nitrogen. Boron trifluoride diethyl etherate (0.44 mL, 3.59 mmol) was added slowly. The −78° C. cold bath was removed and replaced with a 0° C. cold bath. Bright yellow color quickly faded. Reaction is a white cloudy mixture. The reaction mixture was stirred at 0° C. for 2 h. The reaction mixture was partitioned between dichloromethane and saturated aqueous sodium bicarbonate. The water layer was extracted again with dichloromethane. The combined organics were dried over sodium sulfate, filtered, concentrated on a rotary evaporator, and purified via silica gel chromatography (20-100% ethyl acetate in dichloromethane) to obtain (3) as viscous liquid. Yield: 1.5 g, 54.43%; LC-MS m/z 576.5 [M+1]⁺.

To a solution of (3, 1.50 g, 2.61 mmol) in methanol (20.0 mL) was added 10% palladium carbon (0.6 g). The reaction mixture was stirred at room temperature for 1 h under hydrogen atmosphere. After completion, the reaction mixture was filtered through Syringe filter, filtrate was concentrated and dried to get (4) as light pink liquid. Yield: 1.2 g, 84.4%; LC-MS m/z 546.46 [M+1]⁺.

To a solution of (4, 1.20 g, 2.20 mmol) in N,N-dimethyl formamide (15.0 mL) was added N-(hex-5-yn-1-yl)-1H-imidazole-1-carboxamide (4a, 0.505 g, 2.64 mmol) and 4-dimethylaminopyridine (0.269 g, 2.20 mmol). The reaction mixture was stirred at 60° C. for 24 h. After completion, the reaction mixture was diluted with water and extracted with ethyl acetate. The organic layer was dried over sodium sulfate, filtered and concentrated under reduced pressure to get crude. The crude was purified by flash chromatography (silica mesh: 100-200; (elution: 3-5% methanol in dichloromethane) to obtain (5) as a pale yellow sticky liquid. Yield: 1.10 g, 74.78%; LC-MS m/z 669.2 [M+1]⁺.

To a solution of (5, 1.10 g, 1.65 mmol) in acetonitrile (15.0 mL) was added bromotrimethylsilane (1.09 mL, 8.23 mmol) at 0° C. The reaction mixture was stirred at room temperature for 5 h. After completion (monitored by LCMS), the reaction mixture was concentrated under reduced pressure to obtain sticky mass which was triturated with diethyl ether to obtain (6) as crude compound which was used as such for next step without further purification. Yield: 1.0 g (crude); LCMS m/z 613.3 [M+1]⁺.

To a solution of (6, 1.00 g, 1.63 mmol) in methanol (10.0 mL) was added sodium methanolate (0.49 mL, 8.16 mmol) at 0° C. The reaction mixture was stirred at 0° C. to room temperature for 30 min. After completion (monitored by LCMS), the reaction mixture was concentrated under reduced pressure to obtain crude. The crude was purified by prep HPLC using (20-50% acetonitrile in water with 0.1% TFA) to afford {2-[(2R,3S,4S,5S,6R)-6-(4-{[(hex-5-yn-1-yl)carbamoyl]amino}-2-methylphenoxy)-3,4,5-trihydroxyoxan-2-yl]ethyl}phosphonic acid (I-73) as off-white solid. Yield: 0.47 g, 59.94%; 487.5 [M+1]; ¹H NMR (400 MHz, DMSO-d₆) δ 8.13 (s, 1H), 7.18 (d, J=2.0 Hz, 1H), 7.09 (dd, J=2.0, 8.4 Hz, 1H), 6.90 (d, J=8.8 Hz, 1H), 6.03 (t, J=5.2 Hz, 1H), 5.24 (s, 1H), 5.00 (bs, 2H), 4.72 (bs, 1H), 3.83 (s, 1H), 3.64 (d, J=6.0 Hz, 1H), 3.35-3.25 (m, 1H), 3.15 (s, 1H), 3.05 (t, J=6.0 Hz, 2H), 2.66 (s, 1H), 2.18 (t, J=4.0 Hz, 2H), 2.11 (s, 3H), 1.95 (bs, 1H), 1.65-1.58 (m, 1H), 1.47 (s, 6H), 1.23-1.13 (m, 1H).

Synthesis of synthon Compound I-74

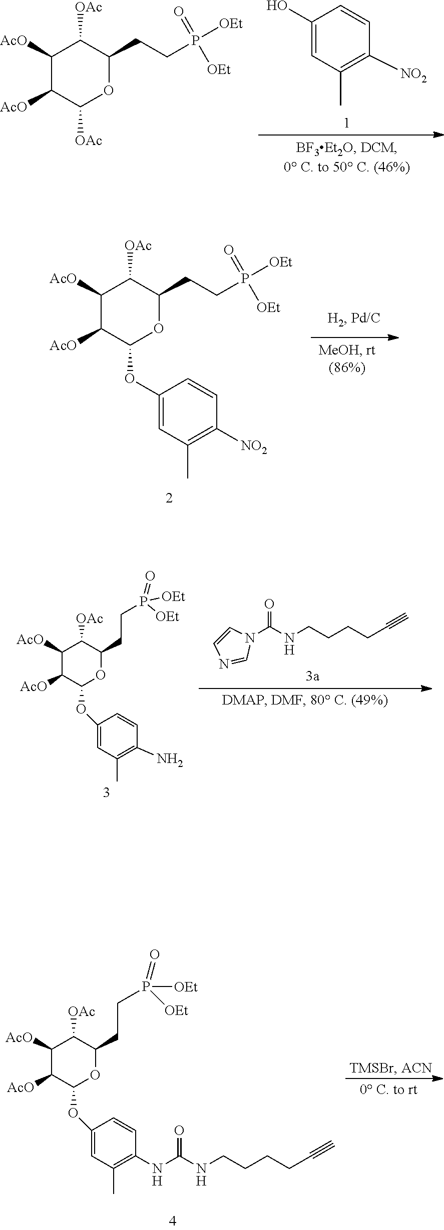

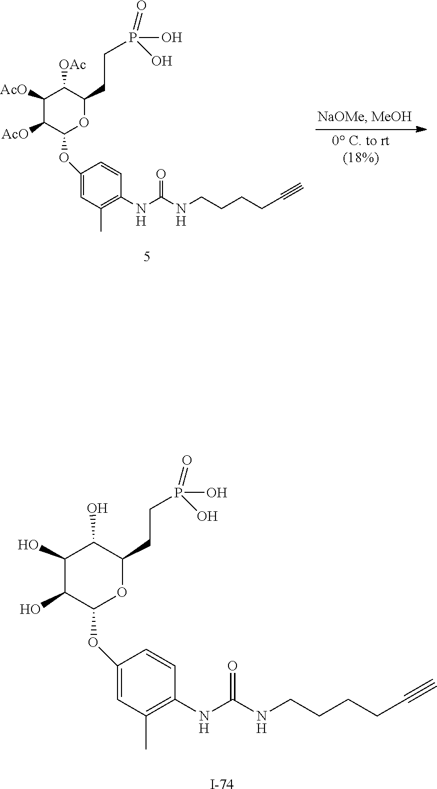

A solution of (2R,3S,4S,5R,6R)-6-(2-(diethoxyphosphoryl)ethyl)tetrahydro-2H-pyran-2,3,4,5-tetrayl tetraacetate (1.0 eq, 2.0 g, 4.15 mmol) and 3-methyl-4-nitrophenol (1, 2.0 eq, 1.27 g, 8.29 mmol) in dichloromethane (20 mL) was cooled at 0° C., boron trifluoride diethyl etherate (5.0 eq, 2.67 mL, 20.7 mmol) was added dropwise and reaction mixture was heated at 50° C. for 16 h. After completion, reaction mixture was cooled at 0° C., quenched with saturated sodium bicarbonate solution and extracted with dichloromethane. Organic layer was dried over anhydrous sodium sulfate, filtered and concentrated to get crude which was purified by column chromatography using silica gel (100-200 mesh) and 0-40% ethyl acetate in dichloromethane to afford (2) as a brown viscous liquid. Yield: 1.1 g, 46.1%; LCMS m/z 576.35 [M+1]⁺.

To a solution of (2, 1.0 eq, 1.1 g, 1.91 mmol) in methanol (11 mL), Palladium on carbon (10%) (0.500 g) was added and reaction mixture was stirred under hydrogen gas atmosphere at room temperature for 2 h. After completion, reaction mixture was filtered, filtrate was concentrated and dried to afford (3) as a brown viscous liquid. Yield: 0.900 g, 86.41%; LCMS m/z 546.29 [M+1]⁺.

To a solution of (3, 1.0 eq, 0.600 g, 1.10 mmol) in N,N-dimethylformamide (6 mL), N-(hex-5-yn-1-yl)-1H-imidazole-1-carboxamide (3a, 1.2 eq, 0.252 g, 1.32 mmol) and 4-dimethylaminopyridine (1.0 eq, 0.134 g, 1.10 mmol) were added and reaction mixture was heated at 80° C. for 16 h. After completion, reaction mixture was cooled, water was added and extracted with ethyl acetate. Organic layer was washed with water, dried over anhydrous sodium sulphate, filtered and concentrated to get crude which was purified by column chromatography using silica gel (100-200 mesh) and 0-5% methanol in dichloromethane to afford (4) as a colourless viscous liquid. Yield: 0.380 g, 49.29%; LCMS m/z 669.47 [M+1]⁺.

A solution of (4, 1.0 eq, 0.600 g, 0.897 mmol) in dichloromethane (12 mL) was cooled at 0° C., bromotrimethylsilane (8.0 eq, 0.94 mL, 7.18 mmol) were added and reaction mixture was stirred at room temperature for 9 h. Reaction was monitored by LCMS. After completion, reaction mixture was concentrated and dried to afford (5) as a brown viscous liquid. Yield: 0.590 g (Crude); LCMS m/z 613.27 [M+1]⁺.

A solution of (5, 1.0 eq, 0.590 g, 0.963 mmol) in methanol (6 mL) was cooled at 0° C., sodium methoxide (25% solution in methanol) (10.0 eq, 2.36 mL, 9.63 mmol) was added and reaction mixture was stirred at room temperature for 1 h. After completion, reaction mixture was concentrated to get crude which was diluted with acetonitrile and purified by prep HPLC (23-41% acetonitrile in water with 0.1% TFA). Fractions containing the desired product were combined and lyophilized to dryness to afford (2-((2R,3S,4S,5S,6R)-6-(4-(3-(hex-5-yn-1-yl)ureido)-3-methylphenoxy)-3,4,5-trihydroxytetrahydro-2H-pyran-2-yl)ethyl)phosphonic acid (I-74) as an off white solid. Yield: 0.085 g, 18.11%; LCMS m/z 487.13 [M+2]++; ¹H NMR (400 MHz, DMSO-d₆) δ 7.56-7.53 (m, 1H), 7.46 (s, 1H), 6.83 (s, 1H), 6.79-6.76 (m, 1H), 6.35-6.34 (m, 1H), 5.25 (s, 1H), 4.99-4.73 (m, 2H), 3.78 (s, 1H), 3.61-3.59 (m, 1H), 3.35-3.30 (m, 2H), 3.07-3.06 (m, 2H), 2.77-2.75 (m, 1H), 2.18 (bs, 2H), 2.13 (s, 3H), 1.96-1.95 (m, 1H), 1.60-1.57 (m, 1H), 1.48 (s, 5H), 1.23-1.14 (m, 1H).

### Synthesis of Synthon Compound I-75

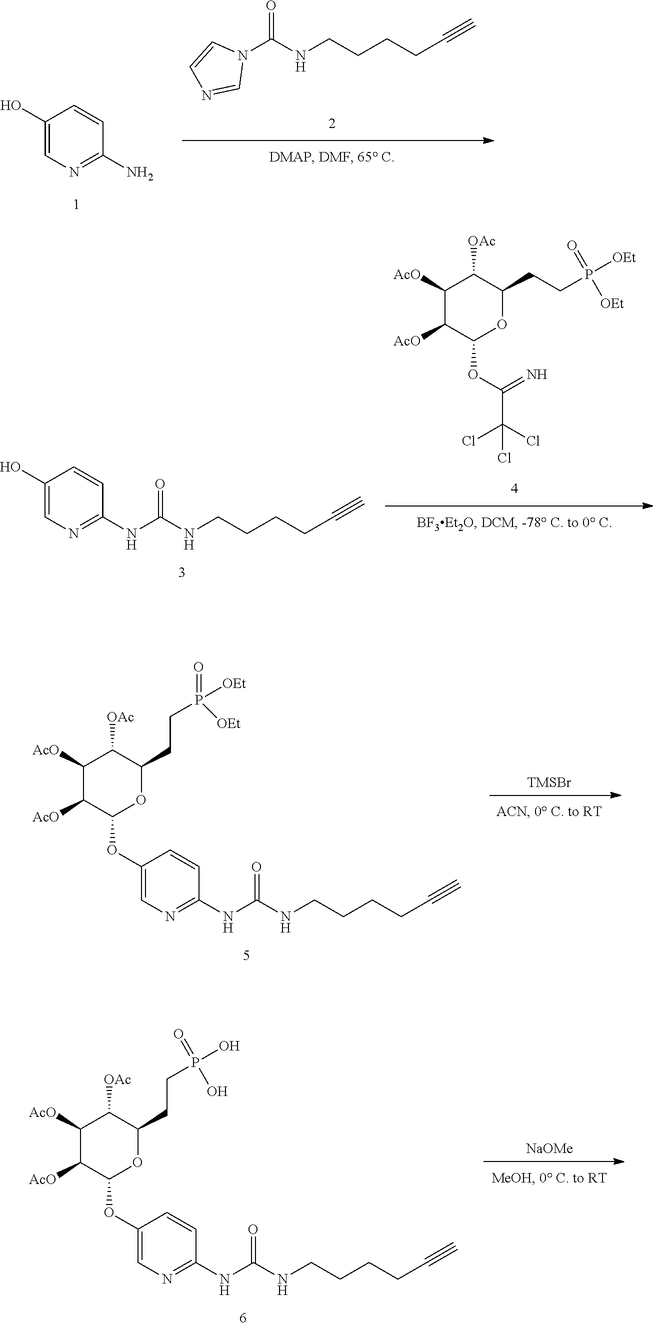

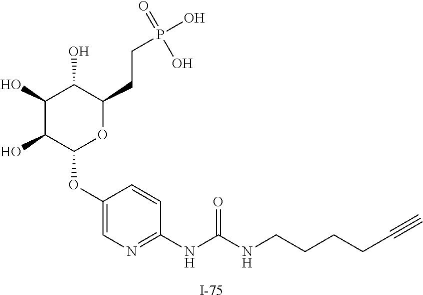

To a solution of 6-aminopyridin-3-ol (1, 1.5 g, 13.6 mmol) in N,N-dimethyl formamide (15.0 mL) is added N-(hex-5-yn-1-yl)-1H-imidazole-1-carboxamide (2, 2.6 g, 13.6 mmol) and N,N-dimethylpyridin-4-amine (1.66 g, 13.6 mmol). The reaction mixture is heated at 65° C. for 16 h. After completion, the reaction mixture is concentrated under reduced pressure to obtain crude. The crude is purified by column chromatography (silica mesh: 100-200; elution: 2-5% methanol in dichloromethane) to afford 3-(hex-5-yn-1-yl)-1-(4-hydroxyphenyl)urea (3).

In an inert atmosphere (4, 1.0 g, 1.71 mmol) is dissolved in dry dichloromethane (10.0 mL) and stirred at room temperature. 3-(hex-5-yn-1-yl)-1-(4-hydroxyphenyl)urea (3, 0.4 g, 1.71 mmol) is added to the former solution and the resulting clear solution is cooled to −78° C. with stirring under nitrogen. Boron trifluoride diethyl etherate (0.21 mL, 1.71 mmol) is added drop-wise to the reaction vessel and the −78° C. cold bath is replaced with a 0° C. cold bath. The reaction mixture is stirred at 0° C. for 4 h and progress of reaction monitored with TLC and LC-MS. After completion, the reaction mixture is quenched with saturated aqueous sodium bicarbonate at 0° C. and partitioned between dichloromethane and aqueous layer. The aqueous layer is extracted again with dichloromethane (2×10 mL). The separated organic layers are combined, dried over anhydrous sodium sulfate, filtered, concentrated on a rotary evaporator and purified by silica gel column chromatography (10% methanol in dichloromethane) to obtain (5).

To a solution of (5, 1.0 eq) in acetonitrile (10 vol.) is added bromotrimethylsilane (5.0 eq) at 0° C. The reaction mixture is stirred at room temperature for 5 h and progress monitored by TLC and LC-MS. After completion, the reaction mixture is concentrated under reduced pressure to obtain crude mass. The crude is washed with diethyl ether and decanted to obtain (6).

To a solution of (6, 1.0 eq) in methanol (10 vol.) is added sodium methoxide (10.0 eq) at 0° C. The reaction mixture is stirred at room temperature for 30 minutes and progress monitored by TLC. After completion, the reaction mixture is concentrated under reduced pressure to get crude. The crude is purified by prep-HPLC to afford dibenzyl (2-((2R,3S,4S,5S,6R)-6-((6-(3-(hex-5-yn-1-yl)ureido)pyridin-3-yl)oxy)-3,4,5-trihydroxytetrahydro-2H-pyran-2-yl)ethyl)phosphonic acid (I-75).

### Synthesis of Synthon Compound I-76

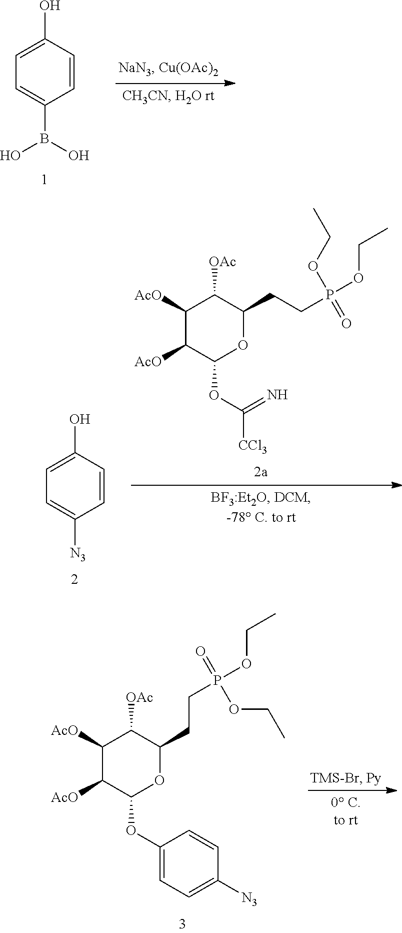

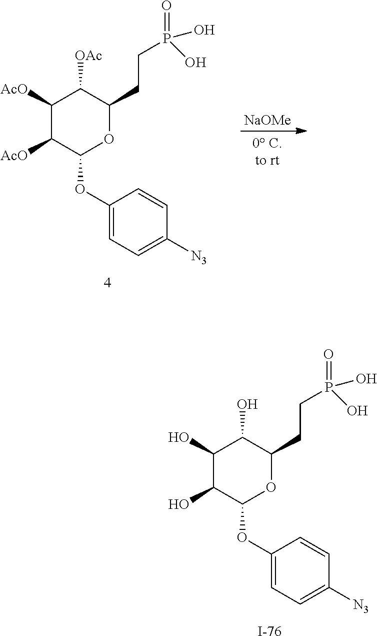

To a solution of compound (4-hydroxyphenyl)boronic acid (1, 3.00 g, 1 eq, 21.8 mmol) and Sodium azide (2.12 g, 1.5 eq, 32.6 mmol) in mixture of acetonitrile (18.0 mL) and water (18.0 mL) was added copper(II) acetate (0.39 g, 0.1 eq, 32.6 mmol) and reaction mixture stirred at room temperature under air for 16 h. Reaction mixture partitioned in between ethyl acetate and water. Ethyl acetate layer separated and aqueous layer re-extracted with ethyl acetate. Combined ethyl acetate layer washed with brine solution, dried over anhydrous sodium sulphate, filtered and concentrated under reduced pressure to get crude product. Crude product obtained was purified by flash column chromatography on silica gel column eluting product in 20 to 30% ethyl acetate in hexane as eluents. Desired fractions were concentrated under reduced pressure to afford 4-azidophenol (2) as brownish sticky gum. Yield: 1.80 g, 61%; LCMS m/z 194.23 [M+60]-.

To a solution of 4-azidophenol (2, 0.324 g, 2.0 eq, 2.39 mmol) and [(2R,3R,4S,5S,6R)-4,5-diacetoxy-2-(2-diethoxyphosphorylethyl)-6-(2,2,2-trichloroethanimidoyl)oxy-tetrahydropyran-3-yl]acetate (2a, 0.700 g, 1.0 eq, 1.20 mmol) in dry dichloromethane (10 mL) at −78 C, Boron trifluoride diethyl etherate (0.111 mL, 0.75 eq, 0.898 mmol) was added slowly and reaction mixture was allowed to come at room temperature and stirred for 16 h. The reaction mixture was partitioned between dichloromethane and saturated aqueous sodium bicarbonate. The aqueous layer was re-extracted again with dichloromethane. The combined organics were dried over anhydrous sodium sulfate, filtered, and evaporated under reduced pressure to get crude residue. Crude product obtained was purified by flash column chromatography on silica gel column eluting product in 40 to 50% ethyl acetate in dichloromethane as eluents to afford (3) as brownish sticky gum. Yield: 0.45 g, 67.43%; LCMS m/z 558.19 [M+1]⁺.

To a solution of (3, 0.450 g, 1.0 eq, 0.807 mmol) in dichloromethane (10.0 mL) at 0° C. were added pyridine (0.977 mL, 15 eq, 12.1 mmol) and bromotrimethylsilane (1.07 mL, 10 eq, 8.07 mmol) and reaction mixture was stirred at room temperature for 4 h. LCMS showed consumption of starting material. Reaction mixture cooled to 0° C. and quenched by addition of cold water. Dichloromethane layer separated and Aqueous layer re-extracted with dichloromethane, combined dichloromethane layer dried over anhydrous sodium sulphate, filtered and concentrated under reduced pressure to afford (4) as brownish sticky gum. Yield: 0.45 g, 80%; LCMS m/z 500.23 [M−1]⁻

To a solution of (4, 0.405 g, 1.0 eq, 0.807 mmol) in methanol (5.0 mL) at 0° C. was added sodium methanolate (25% solution, 0.533 mL, 3 eq, 2.42 mmol) and reaction mixture stirred at room temperature for 1 h. LCMS showed consumption of Starting material. Reaction mixture neutralized with Dowex 50WX8 hydrogen form and filtered over sintered funnel. Filtrate obtained was concentrated under reduced pressure to get crude product. Crude product obtained was purified by reverse phase preparative HPLC using 13% to 35% acetonitrile in water with 0.1% trifluoroacetic acid to afford (2-((2R,3S,4S,5S,6R)-6-(4-azidophenoxy)-3,4,5-trihydroxytetrahydro-2H-pyran-2-yl)ethyl)phosphonic acid (I-76) as cream color solid. Yield: 0.172 g, 56.78%; LCMS m/z 376.15 [M+1]. ¹H NMR (400 MHz, DMSO-d6) δ 10.15 (bs, 1H), 7.10-7.04 (m, 5H), 5.05-4.77 (bm, 3H), 3.81 (s, 1H), 3.61 (d, J=8.0 Hz, 1H), 3.35-3.22 (m, 3H), 1.95-1.92 (bm, 1H), 1.61-1.45 (m, 2H), 1.17-1.05 (m, 1H).

### Synthesis of Synthon I-77

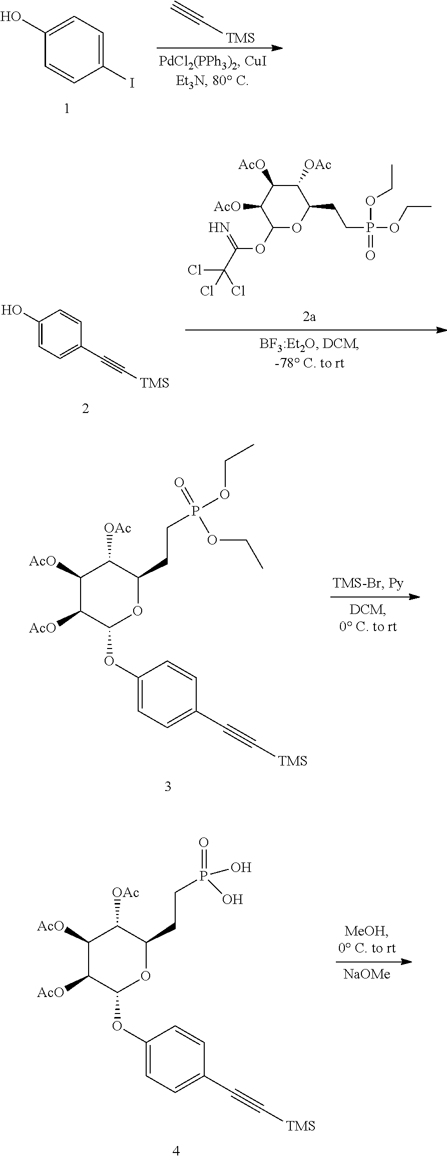

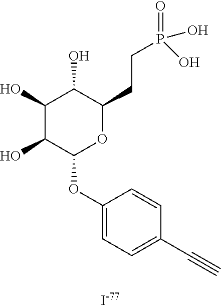

To a solution of 4-Iodophenol (1, 3.0 g, 1.0 eq, 13.6 mmol, 1 eq) in triethylamine (54.0 mL), copper (I) iodide (0.077 g, 0.409 mmol, 0.03 eq) was added and nitrogen gas was purged in reaction mixture for 10 minutes. Bis(triphenylphosphine)palladium(II) dichloride (0.287 g, 0.409 mmol, 0.03 eq), and trimethylsilylacetylene (3.0 mL, 20.5 mmol, 1.5 eq) were subsequently added into reaction mixture and reaction mixture heated at 80° C. for 3 h. Reaction mixture cooled down and concentrated under reduced pressure to get crude residue. Crude residue obtained was purified by flash column chromatography using silica gel column and 10 to 20% Ethyl acetate in hexane as eluents. Desired fractions were concentrated under reduced pressure to afford 4-[2-(trimethylsilyl)ethynyl]phenol (2) as brownish sticky gum. Yield: 2.58 g (99%); LCMS m/z 189.07 (M−1)⁻.

To a solution of (2R,3R,4S,5S,6R)-3,5-bis(acetyloxy)-2-[2-(diethoxyphosphoryl) ethyl]-6-[(2,2,2-trichloroethanimidoyl) oxy]oxan-4-yl acetate (2a, 1.40 g, 1.0 eq, 2.39 mmol) in dry dichloromethane (20.0 mL), 4-[2-(trimethylsilyl)ethynyl]phenol (2, 0.911 g, 2.0 eq, 4.79 mmol) was added and resulting solution was cooled to −78° C. Boron trifluoride diethyl etherate (0.222 mL, 0.75 eq, 1.80 mmol) was added slowly and reaction mixture was allowed to come at room temperature and stirred for 16 h. After completion of reaction, reaction mixture cooled down and partitioned in between dichloromethane and aqueous sodium bicarbonate solution. Dichloromethane layer separated and aqueous layer was re-extracted with dichloromethane. The combined organic layer was dried over anhydrous sodium sulfate, filtered, concentrated under reduce pressure, and purified by flash column chromatography using silica gel column and 20 to 30% ethyl acetate in dichloromethane as eluents. Desired fractions were concentrated under reduced pressure to obtain (3) as pale yellow sticky gum. Yield: 0.710 g, 48.4%; LCMS m/z 613.28 [M+1]⁺.

To a solution of (3, 0.610 g, 1.0 eq, 0.996 mmol) in dichloromethane (15.0 mL) at 0° C. were added pyridine (1.21 mL, 15 eq, 14.9 mmol) and bromotrimethylsilane (1.31 mL, 10 eq, 9.96 mmol) and reaction mixture stirred at room temperature for 4 h. LCMS showed consumption of starting material. Reaction mixture cooled to 0° C. and quenched by addition of cold water. Dichloromethane layer separated and aqueous layer re-extracted with dichloromethane. combined dichloromethane layer dried over anhydrous sodium sulphate and concentrated under reduced pressure to afford (4) as brownish sticky gum. Yield:0.51 g, 92.3%; LCMS m/z 555.38 [M−1]⁻

To a solution of (4, 0.510 g, 1.0 eq, 0.916 mmol) in methanol (8.00 mL) at 0° C. was added sodium methanolate (0.605 mL, 3 eq, 2.75 mmol) and reaction mixture stirred at room temperature for 4 h. Reaction mixture cooled and quenched by addition of Dowex 50W X8 hydrogen form up to pH 6 and filtered over sintered funnel. Filtrate obtained was concentrated under reduced pressure to get crude product. Crude product obtained was purified by reverse phase preparative HPLC using 10 to 35% acetonitrile in water and 0.1% TFA to afford (2-((2R,3S,4S,5S,6R)-6-(4-ethynylphenoxy)-3,4,5-trihydroxytetrahydro-2H-pyran-2-yl)ethyl)phosphonic acid (I-77) as cream color solid. Yield: 0.213 g, 64%; LCMS m/z 359.06 [M+1]. ¹H NMR (400 MHz, DMSO-d6) δ 10.20 (bs, 1H), 7.41 (d, J=8.80 Hz, 2H), 7.03 (d, J=8.80 Hz, 2H), 5.44 (s, 1H), 5.08-4.78 (bm, 3H), 4.05 (s, 1H), 3.81 (s, 1H), 3.62 (d, J=6.40 Hz, 1H), 3.35-3.19 (m, 3H), 1.92 (bs, 1H), 1.60-1.49 (m, 2H), 1.14-1.05 (m, 1H).

### Synthesis of Synthon Compound I-78

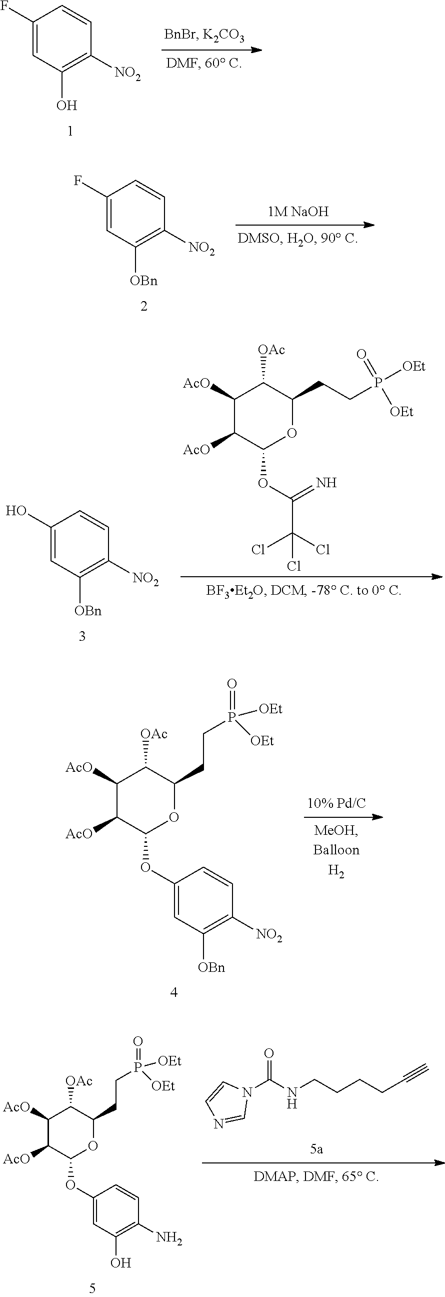

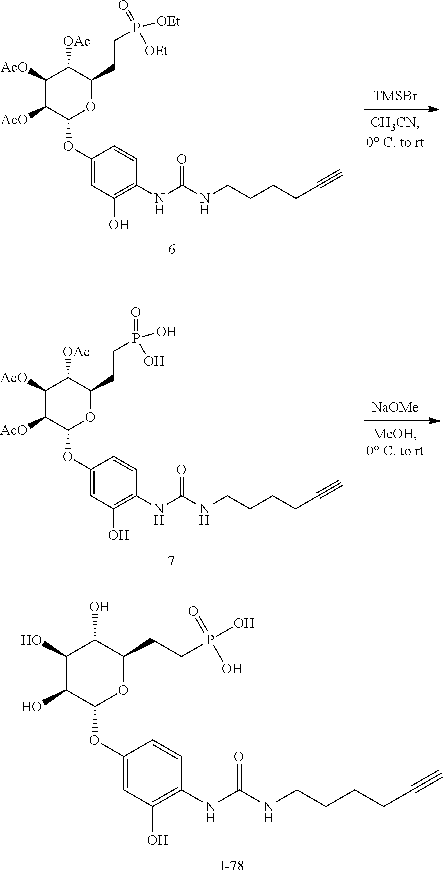

To a solution of 5-fluoro-2-nitrophenol (1, 5.00 g, 1.0 eq, 31.8 mmol) in N, N-dimethylformamide (50.0 mL) were added potassium carbonate (5.28 g, 1.20 eq, 38.2 mmol) and benzyl bromide (4.16 mL, 35.0 mmol) and the reaction mixture was heated at 60° C. for 3 h. After completion, reaction mixture was diluted with water and extracted with ethyl acetate. The organic layer was dried over anhydrous sodium sulfate, filtered and concentrated under reduced pressure to afford 2-(benzyloxy)-4-fluoro-1-nitrobenzene (2) as yellow solid which was used as such for next step without further purification. Yield: 8.0 g, 99.64; LC-MS m/z 248.2 [M+1]⁺.

To a solution of 2-(benzyloxy)-4-fluoro-1-nitrobenzene (2, 7.00 g, 28.3 mmol) in dimethylsulfoxide (35.00 mL) was added 1M sodium hydroxide solution in water (35.0 mL). The reaction mixture was stirred at 80° C. for 18 h. After completion (monitored by TLC), the reaction mixture was acidified with 1M hydrochloric acid (10 mL) until the pH 3-4 and the resultant solution was extracted with ethyl acetate. The organic layer was washed with water, dried over anhydrous sodium sulfate and concentrated to get crude. The crude was purified by flash column chromatography (silica mesh 100-200 mesh) using 15-20% ethyl acetate in hexane to afford 3-(benzyloxy)-4-nitrophenol (3) as yellow solid. Yield: 4.10 g, 59.05%. LC-MS m/z 246.2 [M+1]⁺.

(3a, 0.25 g, 1.0 eq 0.428 mmol) was dissolved in dry dichloromethane (2.5 mL) with stirring under nitrogen. 3-(benzyloxy)-4-nitrophenol (3, 0.105 g, 1.0 eq, 0.428 mmol) was added and the resulting clear solution was cooled to −78° C. with stirring under nitrogen. Boron trifluoride diethyl etherate (0.052 mL, 1.0 eq, 0.428 mmol) was added slowly. The −78° C. cold bath was removed and replaced with a 0° C. cold bath. The reaction mixture was stirred at 0° C. for 2 h. The reaction mixture was partitioned between dichloromethane and saturated aqueous sodium bicarbonate. The water layer was extracted again with dichloromethane. The combined organics were dried over anhydrous sodium sulfate, filtered, concentrated on a rotary evaporator, and purified via silica gel chromatography (5-10% methanol in dichloromethane) to obtain (4) as viscous liquid. Yield: 0.12 g (~65% purity by LCMS); LC-MS m/z 668.6 [M+1]⁺.

To a solution of (4, 1.0 eq) in methanol (10 vol.) is added 10% palladium on carbon (quant.). The reaction mixture is stirred at room temperature for 3 h under hydrogen atmosphere. After completion, the reaction mixture was filtered through Syringe filter, filtrate is concentrated and dried to get (5). LC-MS m/z 548.15 [M+1]⁺.

To a solution of (5, 1.0 eq) in N,N-dimethyl formamide (10 vol) is added N-(hex-5-yn-1-yl)-1H-imidazole-1-carboxamide (5a, 1.2 eq) and 4-dimethylaminopyridine (1.0 eq). The reaction mixture is stirred at 60° C. for 24 h. After completion, the reaction mixture is diluted with water and extracted with ethyl acetate. The organic layer is dried over anhydrous sodium sulfate, filtered and concentrated under reduced pressure to get crude. The crude is purified by flash chromatography (silica mesh: 100-200) and 5 to 10% methanol in dichlormethane as eluents to afford (6). LC-MS m/z 671.25 [M+1]⁺.

To a solution of (6, 1.0 eq) in acetonitrile (10 vol.) is added bromotrimethylsilane (5.0 eq) at 0° C. The reaction mixture is stirred at room temperature for 5 h. After completion, the reaction mixture is concentrated under reduced pressure to obtain sticky mass which is triturated with diethyl ether to obtain (7) as crude compound which is used as such for next step without further purification. LC-MS m/z 615.15 [M+1]⁺.

(7, 1.0 eq) in methanol (10 vol.) is added sodium methanolate (10.0 eq) at 0° C. The reaction mixture is stirred at 0° C. to room temperature for 30 min. After completion, the reaction mixture is neutralized by Dowex 50WX8 hydrogen form up to pH 6 to 7 and filtered. Filtrate is concentrated under reduced pressure to obtain crude. The crude is purified by reverse phase preparative HPLC to afford (2-((2R,3S,4S,5S,6R)-6-(4-(3-(hex-5-yn-1-yl)ureido)-3-hydroxyphenoxy)-3,4,5-trihydroxytetrahydro-2H-pyran-2-yl)ethyl)phosphonic acid (I-78). LC-MS m/z 489.16 [M+1]⁺.

### Synthesis of Synthon Compound I-79

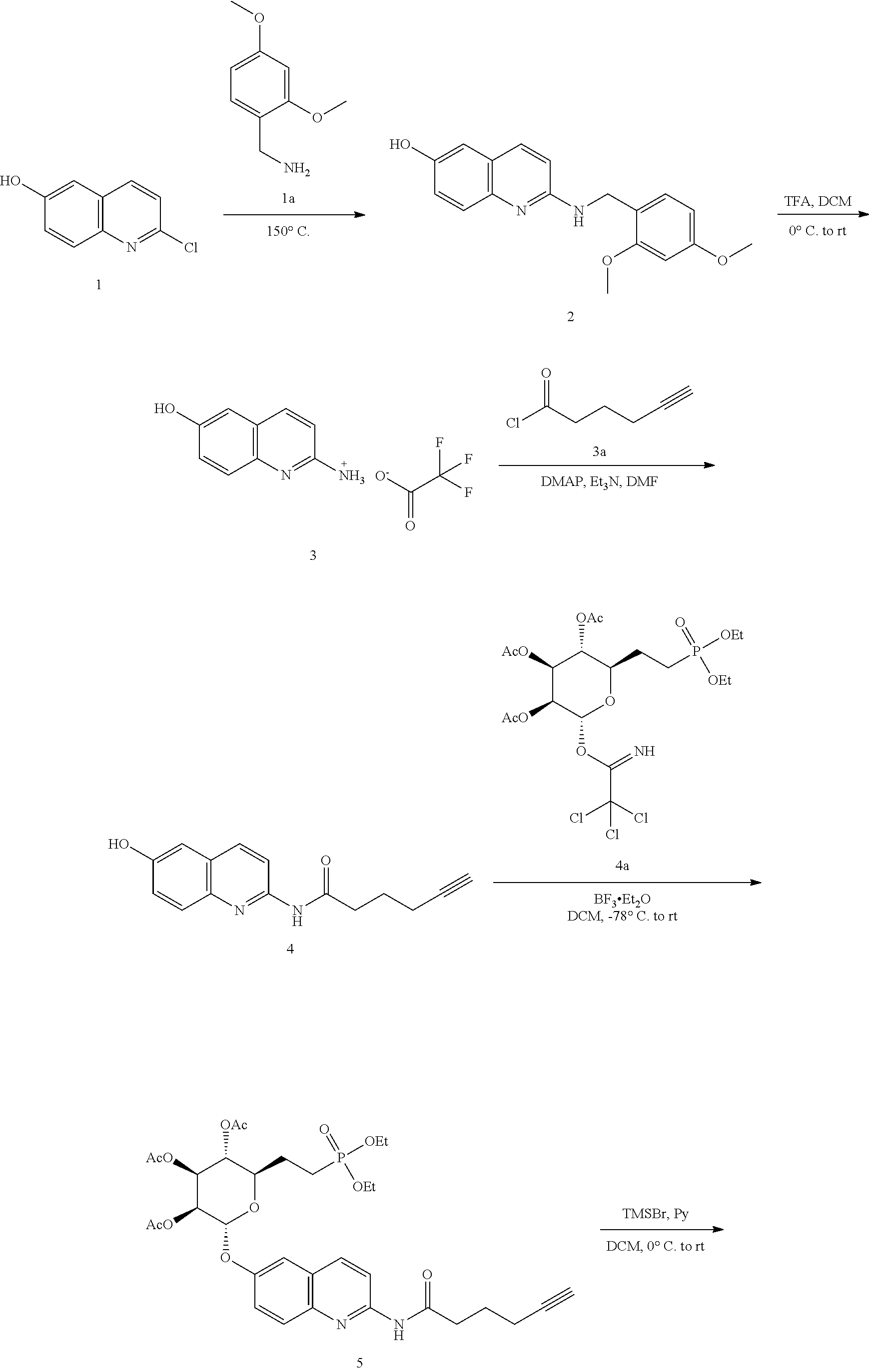

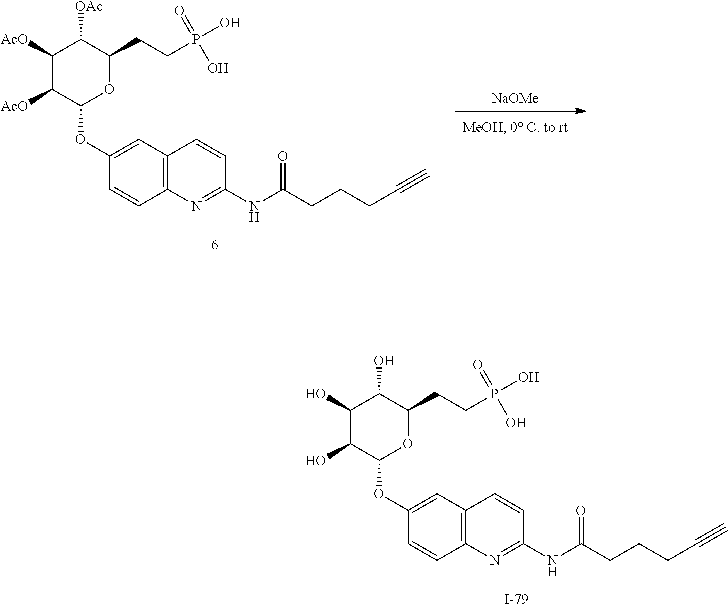

A solution of 2-chloroquinolin-6-ol (1, 1.0 g, 1.0 eq, 5.57 mmol) and (2,4-dimethoxyphenyl)methanamine (1a, 1.67 mL, 2.0 eq, 11.1 mmol) is heated at 150° C. for 16 h and progress of reaction is checked by TLC and LC-MS. After completion, reaction is concentrated and observed crude residue is purified by combiflash chromatography using silica gel column and 30 to 40% ethyl acetate in hexane as eluents to afford 2-((2,4-dimethoxybenzyl)amino)quinolin-6-ol (2).

To a solution of 2-((2,4-dimethoxybenzyl)amino)quinolin-6-ol (2, 0.10 g, 0.32 mmol) in dichloromethane (0.5 mL) at 0° C. is added trifluoroacetic acid (0.5 mL) and reaction mixture stirred at room temperature for 6 h. Reaction mixture concentrated under reduced pressure to afford 2-aminoquinolin-6-ol trifluro acetic acid salt (3).

To a solution of 2-aminoquinolin-6-ol trifluro acetic acid salt (3, 1.0 eq.) in N,N-dimethylformamide is added triethyl amine (0.12 mL, 3.0 eq, 0.87 mmol) and N,N-dimethylpyridin-4-amine (0.2 eq.). Reaction mixture is cooled to 0° C. and hex-5-ynoyl chloride (3a, 0.045 g, 1.2 eq, 0.34 mmol) is added to reaction mixture and stirred for 16 h and monitored by TLC and LC-MS for the completion. Reaction mixture partitioned in between ethyl acetate and water. Ethyl acetate layer separated and aqueous layer re-extracted with ethyl acetate. Ethyl acetate layer is dried over anhydrous sodium sulfate and concentrated to get crude residue. Crude residue obtained is purified by flash chromatography using silica gel column and 20 to 50% ethyl acetate in hexane as eluent to afford N-(6-hydroxyquinolin-2-yl)hex-5-ynamide (4).

To a solution of (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (4a, 1.0 eq.) in dry dichloromethane, N-(6-hydroxyquinolin-2-yl)hex-5-ynamide (4, 2.0 eq.) is added and resulting solution is cooled to −78° C. Boron trifluoride diethyl etherate (0.75 eq) is added slowly and reaction mixture is allowed to come at room temperature and stirred for 16 h. After completion of reaction, reaction quenched with saturated aqueous sodium bicarbonate solution and partitioned in between dichloromethane and aqueous phase. Aqueous layer re-extracted with dichloromethane, the combined organic layer is dried over anhydrous sodium sulfate, filtered, concentrated under reduce pressure, and purified by flash column chromatography using silica gel column to obtain (5).

To a solution of (5, 1.0 eq) in dichloromethane at 0° C. are added pyridine (15 eq) and bromotrimethylsilane (10 eq) and reaction mixture is stirred at room temperature for 4 h. LCMS showed consumption of starting material. Reaction mixture is cooled to 0° C. and quenched by addition of cold water. Dichloromethane layer is separated and aqueous layer re-extracted with dichloromethane. combined dichloromethane layer is dried over anhydrous sodium sulphate and concentrated under reduced pressure to afford (6).

To a solution of (6, 1.0 eq.) in methanol at 0° C. is added sodium methanolate (3.0 eq.) and reaction mixture is stirred at room temperature for 4 h. Reaction mixture cooled and quenched by addition of Dowex® 50W X8 hydrogen form up to neutral pH and filtered through sintered funnel. Filtrate obtained is concentrated under reduced pressure to get crude product. Crude product obtained is purified by reverse phase preparative HPLC to afford (2-((2R,3S,4S,5S,6R)-6-(4-ethynylphenoxy)-3,4,5-trihydroxytetrahydro-2H-pyran-2-yl)ethyl)phosphonic acid (I-79).

### Synthesis of Synthon Compound I-80

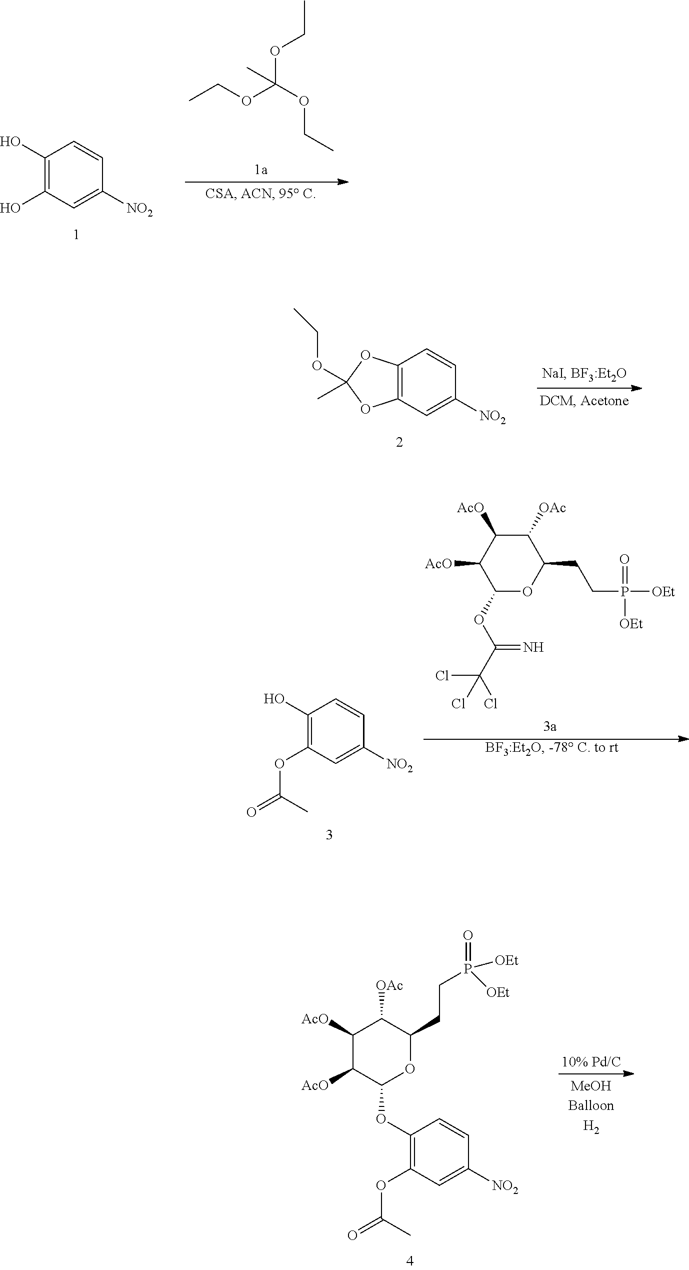

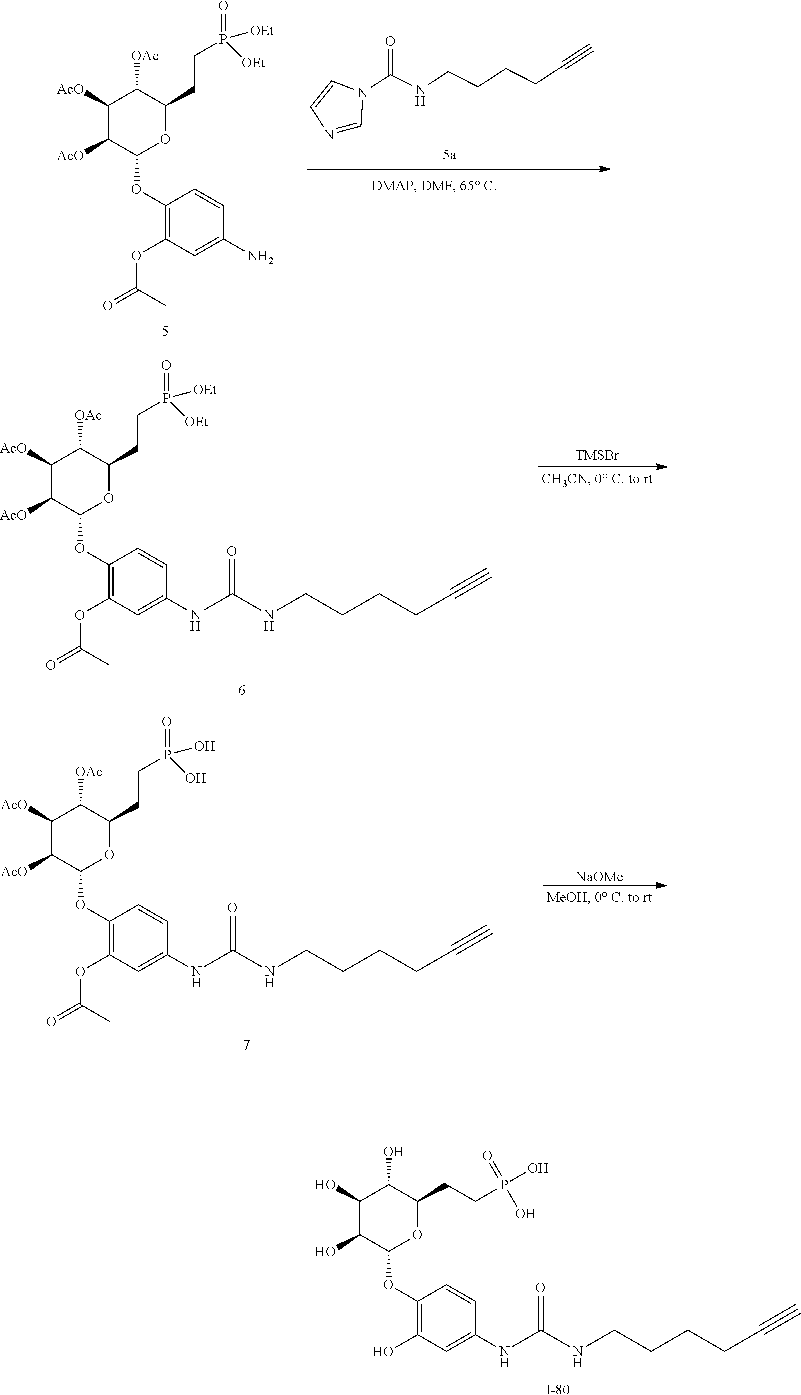

To a solution 4-nitrobenzene-1,2-diol (1, 2.0 g, 12.9 mmol) in acetonitrile (20.0 mL) were added camphor sulfonic acid (0.449 g, 0.019 mmol) and 1,1,1-triethoxyethane (23.8 mL, 129 mmol). The reaction mixture was stirred at 95° C. for 18 h. After completion (monitored by TLC), the reaction mixture was concentrated to get crude which was purified by column chromatography (100-200 mesh silica) using 0-10% ethyl acetate in hexane to afford 2-methoxy-2-methyl-5-nitro-2H-1,3-benzodioxole (2) as white solid. Yield: 1.0 g, 34.44%. LCMS m/z 226.07 [M+1]⁺.

To a solution of 2-ethoxy-2-methyl-5-nitrobenzo[d][1,3]dioxole (2, 1.00 g, 1.0 eq, 4.4 mmol) in dichloromethane (5 mL) at 0° C. is added an anhydrous solution of sodium iodide (1.97 g, 3 equiv, 13.2 mmol) in acetone (5.0 mL), and boron trifluoride etherate (0.72 mL, 1.33 eq., 5.85 mmol) under nitrogen. After 5 min at 0° C., water (20 mL) and dichloromethane (20 mL) are added. The layers are separated, and after back-extraction of the water layer, the combined dichloromethane layer is dried over anhydrous sodium sulfate, filtered and concentrated to afford 2-hydroxy-5-nitrophenyl acetate (3). LCMS m/z 198.09 [M+1]⁺.

To a solution of [(2R,3R,4S,5S,6R)-4,5-diacetoxy-2-(2-diethoxyphosphorylethyl)-6-(2,2,2-trichloroethanimidoyl)oxy-tetrahydropyran-3-yl]acetate (3a, 1.0 g, 1.0 eq, 1.71 mmol) in dry dichloromethane (10 mL) with stirring under nitrogen. 2-hydroxy-5-nitrophenyl acetate (3, 0.33 g, 1.0 eq, 1.71 mmol) is added and the resulting clear solution is cooled to −78° C. with stirring under nitrogen. Boron trifluoride diethyl etherate (0.24 g, 1.0 eq, 1.71 mmol) is added slowly. The −78° C. cold bath is removed and replaced with a 0° C. cold bath. The reaction mixture is stirred at 0° C. for 2 h. The reaction mixture is partitioned between dichloromethane and saturated aqueous sodium bicarbonate. The water layer is extracted again with dichloromethane. The combined organics is dried over anhydrous sodium sulfate, filtered, and concentrated on a rotary evaporator, and purified via silica gel chromatography to afford (4). LCMS m/z 620.17 [M+1]⁺.

To a solution of (4, 0.50 g, 1.0 eq, 0.807 mmol) in methanol (5.0 mL) is added 10% palladium on carbon (0.20 g). The reaction mixture is stirred at room temperature for 3 h under hydrogen atmosphere. After completion, the reaction mixture is filtered through syringe filter, filtrate is concentrated and dried to afford (5). LCMS m/z 590.14 [M+1]⁺.

To a solution of (5, 0.50 g, 1.0 eq, 0.84 mmol) in N,N-dimethyl formamide (5.0 mL) is added N-(hex-5-yn-1-yl)-1H-imidazole-1-carboxamide (5a, 0.192 g, 1.2 eq, 1.008 mmol) and 4-dimethylaminopyridine (0.102 g, 1.0 eq, 0.84 mmol). The reaction mixture is stirred at 60° C. for 24 h. After completion, the reaction mixture is diluted with water and extracted with ethyl acetate. The organic layer is dried over anhydrous sodium sulfate, filtered and concentrated under reduced pressure to get crude product. The crude is purified by flash chromatography (silica mesh: 100-200) to afford (6). LCMS m/z 713.16 [M+1]⁺.

To a solution of (6, 0.50 g, 1.0 eq, 0.702 mmol) in acetonitrile (5.0 mL.) is added bromotrimethylsilane (0.46 mL, 5.0 eq, 3.51 mmol) at 0° C. The reaction mixture is stirred at room temperature for 5 h. After completion, the reaction mixture is concentrated under reduced pressure to obtain sticky mass which is triturated with diethyl ether to obtain (7) as crude compound, which is used as such for next step without further purification. LCMS m/z 657.20 [M+1]⁺.

To a solution of (7, 0.50 g, 1.0 eq, 0.76 mmol) in methanol (5.0 mL) is added sodium methanolate (0.49 mL, 10.0 eq, 2.28 mmol) at 0° C. The reaction mixture is stirred at 0° C. to room temperature for 3 h. After completion, the reaction mixture is neutralized with Dowex 50WX8 hydrogen form, filtered and concentrated under reduced pressure to obtain crude. The crude is purified by reverse phase preparative HPLC to afford (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (I-80). LCMS m/z 489.07 [M+1]⁺.

### Synthesis of Synthon Compound I-101

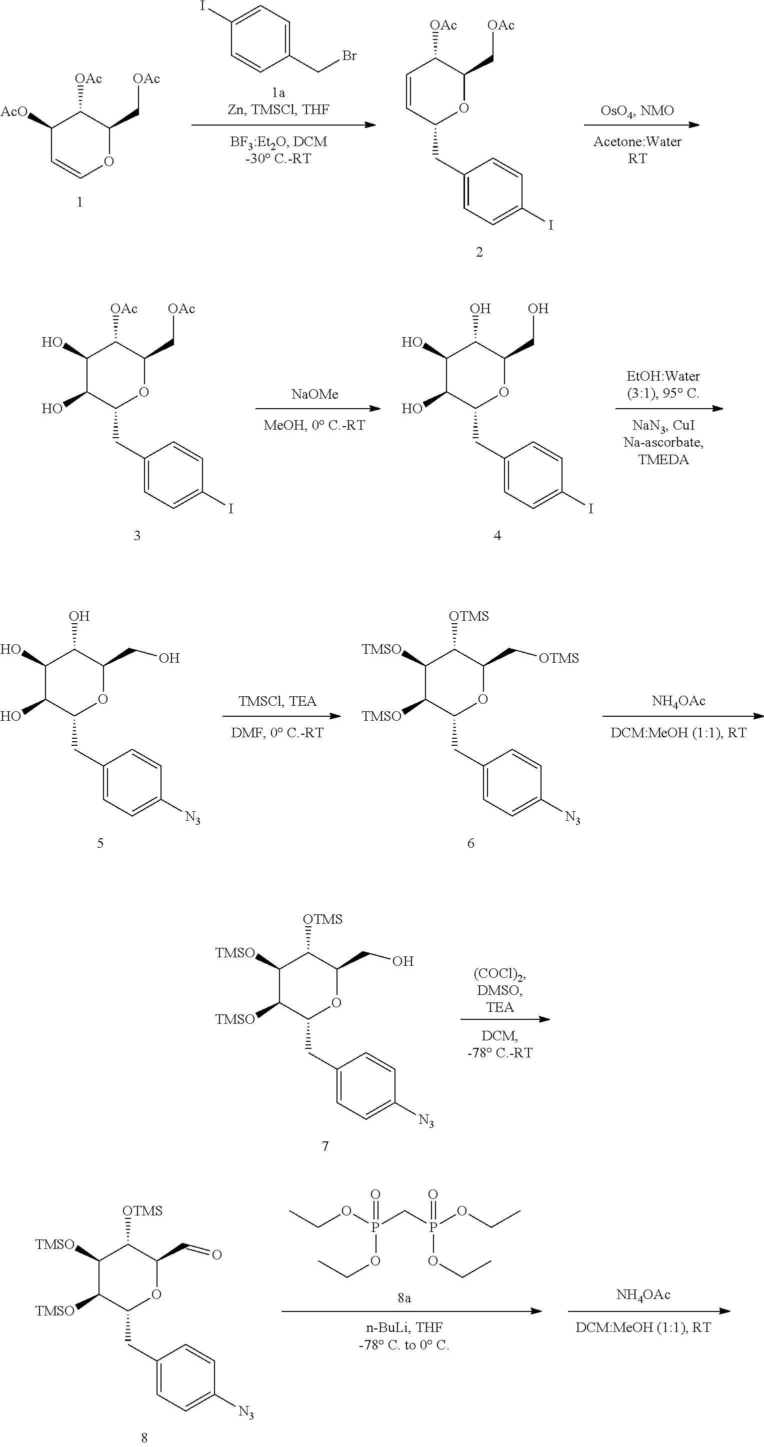

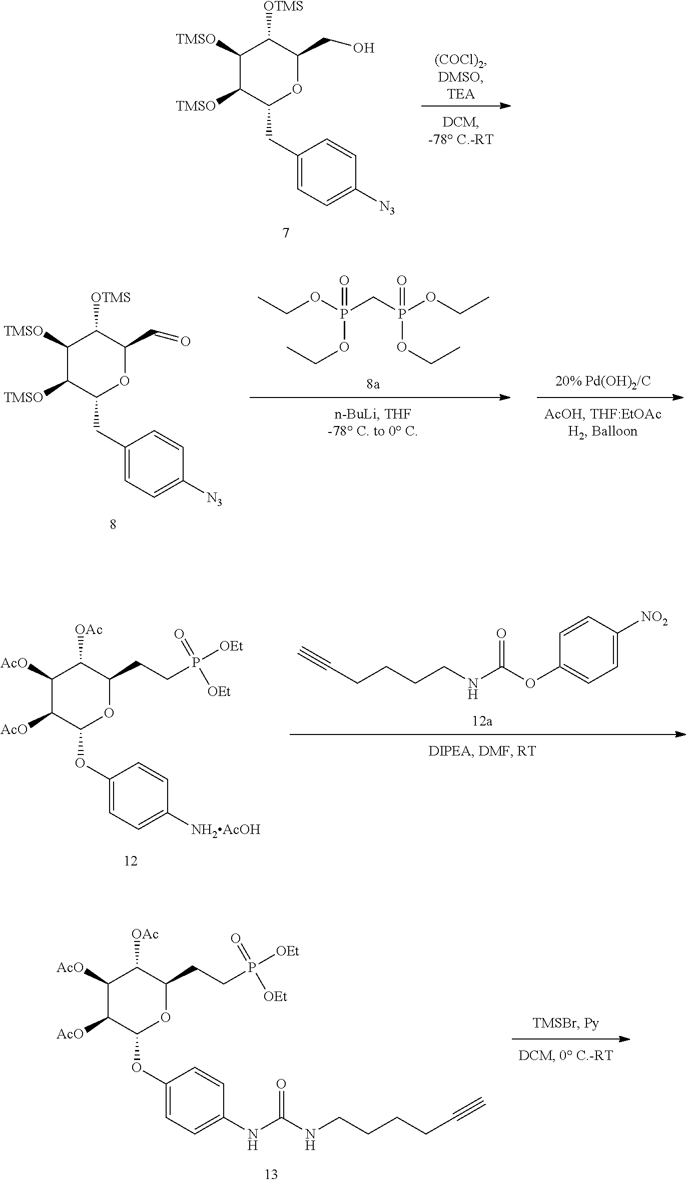

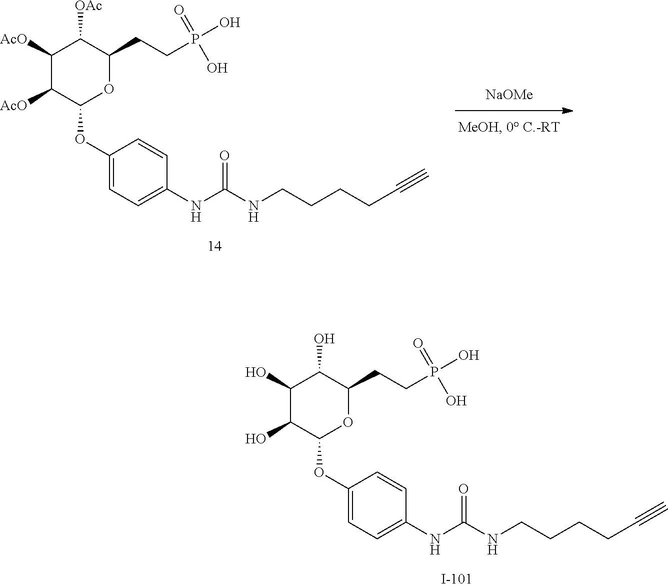 [SMILES: C#CCCCCNC(=O)Nc1ccc(O[C@H]2O[C@H](CCP(=O)(O)O)[C@@H](OC(C)=O)[C@H](OC(C)=O)[C@@H]2OC(C)=O)cc1 ; C#CCCCCNC(=O)Nc1ccc(O[C@H]2O[C@H](CCP(=O)(O)O)[C@@H](O)[C@H](O)[C@@H]2O)cc1 ; "NaOMe"]

Zinc dust (8.01 g, 2.0 eq, 123 mmol) was heated with a heat gun under vacuum for 5 min and cooled to room temperature under vacuum. Dry tetrahydrofuran (10.0 mL) and 1,2-dibromoethane (0.422 mL, 0.08 eq, 4.90 mmol) were added to zinc dust at room temperature and the resulting slurry heated to 60° C. with stirring under nitrogen for 10 min. The slurry cooled to room temperature and chlorotrimethylsilane (0.468 mL, 0.06 eq, 3.69 mmol) added to the previous slurry. The resulting slurry was then stirred for 10 more mins and cooled to 0° C. A solution of 4-iodobenzyl bromide (18.20 g, 1.0 eq, 61.3 mmol) in dry tetrahydrofuran (40.0 mL) was added dropwise, over 1 h, to the stirred suspension of activated zinc at 0° C. under argon in the dark. After addition the mixture was warmed to room temperature and allowed to settle. The zincate solution was transferred away from unreacted zinc via gastight syringe, placed into a flask purged with argon, and the solvent was removed in vacuo (bath temp 35° C.). Dry dichloromethane (40.0 mL) was added to the residue, and the solution was cooled to −30° C. under argon in the dark. A solution of (2R,3S,4R)-2-(acetoxymethyl)-3,4-dihydro-2H-pyran-3,4-diyl diacetate (10.0 g, 0.6 eq, 36.8 mmol) in dry dichloromethane (20.0 mL) was added to the zincate, followed by BF₃:OEt₂ (22.6 mL, 3.0 eq, 184 mmol). The mixture was immediately warmed to 0° C. and stirred for 15 min. The reaction mixture was warmed to room temperature, then diluted with dichloromethane (80 mL), and washed with brine (20 mL); and the organic layer was dried over sodium sulfate, filtered; and the solvent was removed in vacuo. The residue was purified by flash chromatography (ethyl acetate-light petroleum, 1:3) to afford the title compound (2) as a colorless oil. Yield: 7.10 g (44.9%); LCMS, m/z 371.21 [M-OAc]⁺.

N-Methylmorpholine N-oxide (2.25 g, 1.2 eq, 19.2 mmol) and then osmium tetra-oxide (4.0 wt % in water, 10.2 mL, 0.1 eq, 1.60 mmol) were added to a stirred solution of [(2R,3S,6R)-3-(acetyloxy)-6-[(4-iodophenyl)methyl]-3,6-dihydro-2H-pyran-2-yl]methyl acetate (2, 6.90 g, 1.0 eq, 16.0 mmol) in acetone-water (5:1, 80.0 mL) at room temperature. After 24 h, TLC (ethyl acetate-light petroleum, 3:2) indicated no starting material (R_{f}0.8) remained and a new spot generated (R_{f}0.1). Sodium metabisulfite (0.610 g, 0.2 eq, 3.21 mmol) in water (5 mL) was added, and the mixture was stirred vigorously for 0.5 h. Ethyl acetate (50 mL) was added, and the mixture was filtered through Celite into a separating funnel and washed with brine (10 mL). The aqueous layer was extracted with ethyl acetate, and the combined organic fractions were dried over sodium sulfate, filtered and the solvent was removed in vacuo. The residue was purified by flash chromatography (eluent gradient, ethyl acetate-light petroleum, 2:1 to ethyl acetate) to afford (3) as a white solid. Yield: 6.00 g (80.5%); LCMS m/z 482.13 [M+18]⁺.

(3, 6.00 g, 1.0 eq, 12.92 mmol) dissolved in methanol (60.0 mL) and cooled to 0° C. followed by addition of sodium methoxide (0.287 mL, 0.1 eq, 1.29 mmol, 25% w/v solution in methanol). The reaction mixture was stirred at room temperature for 15 min and TLC checked. After completion of reaction, Dowex-50w X8-Hydrogen form added up to neutral pH, the reaction mass was then filtered through sintered and concentrated in vacuo to get (2R,3S,4R,5S,6R)-2-(hydroxymethyl)-6-(4-iodobenzyl)tetrahydro-2H-pyran-3,4,5-triol (4) as off white solid. Yield: 4.10 g (83.4%). LCMS m/z 381.18 [M+H]⁺.

A mixture of (4, 4.0 g, 1.0 eq, 10.5 mmol), diiodocopper (1.67 g, 0.5 eq, 5.26 mmol), sodium azide (1.37 g, 2.0 eq, 21.0 mmol), [2-(dimethylamino)ethyl]dimethylamine (0.476 mL, 0.3 eq, 3.16 mmol) and sodium ascorbate (0.625 g, 0.3 eq, 3.16 mmol) in ethanol:water (50.0 mL, 7:3) in a closed flask was heated to 95° C. under argon and the progress of reaction was monitored by LCMS. After 24 h, reaction was concentrated to dryness under vacuo and the crude was dissolved in methanol, filtered through sintered glass funnel, concentrated, and dried under vacuo to afford (5) as white solid. Yield:3.10 g (99.7%) LCMS m/z 294.57 [M−1].

A stirred solution of (5, 1.0 eq, 3.0 g, 10.16 mmol) in NN-dimethylformamide (40.0 mL) was cooled to 0° C. Then, triethylamine (6.4 eq, 288 mL, 552.0 mmol) and trimethylsilyl chloride (24.0 eq 70 mL, 2071.0 mmol) was added respectively under nitrogen atmosphere to above solution. The resulting mixture was stirred at room temperature under nitrogen for 16 h. The reaction mixture was then partitioned between ethyl acetate and water. The water layer was extracted again with ethyl acetate. The combined organic layers were dried over sodium sulfate, filtered, and purified by silica gel chromatography (0 to 5% ethyl acetate in hexane) to afford) (6) as white solid Yield: 2.78 g (46.3%); LCMS m/z 584.17 [M+1]⁺.

To a stirred solution of (6, 1.0 eq, 2.7 g, 4.62 mmol) in mixture of DCM:MeOH (1:1, 30 mL) ammonium acetate (1.5 eq, 0.534 g, 6.93 mmol) was added at room temperature under nitrogen. The resulting mixture was stirred at room temperature under nitrogen for 16 h. The reaction mixture was then partitioned between ethyl acetate and water. The water layer was extracted again with ethyl acetate. The combined organic layers were dried over sodium sulfate, filtered, concentrated under vacuum and purified via silica gel chromatography (20-30% ethyl acetate in hexane) to afford (7) as thick syrup. Yield: 2.08 g (87%); LCMS m/z 510.13 [M−1].

To a stirred solution of oxalyl chloride (1.1 eq, 0.371 mL, 4.30 mmol) in DCM (5 mL) at −78° C. was added a solution of dimethyl sulfoxide (2.2 eq, 0.611 mL, 8.60 mmol) in dichloromethane (5 mL) over 5 min. After being stirred at −78° C. for 20 min, a solution of (7, 1.0 eq, 2.0 g, 3.91 mmol) in dichloromethane (10 mL) was added to the mixture. The reaction mixture was further stirred at −78° C. for 60 min, followed by addition of triethylamine (5.0 eq, 2.75 mL, 19.5 mmol). The resulting mixture was allowed to reach room temperature over 1 h. The turbid mixture was diluted with dichloromethane and washed with water followed by brine solution. The organic layer was dried over sodium sulfate, filtered, and concentrated under high vacuum to afford (8) as light brown gel. Yield (2.4 g, Crude). which was used directly in the next step.

A stirred suspension of tetraethyl methylenebis(phosphonate) (8a, 1.5 eq, 1.96 mL, 7.06 mmol) in dry tetrahedron (50 mL) was cooled to −78° C. and added n-BuLi solution (1.5 eq, 2.94 ml, 7.06 mmol, 2.4 M in Hexane). The resulting mixture was stirred for 1 h at −78° C., then (8, 1.0 eq, 2.40 g, 4.71 mmol) in dry tetrahedron (10 mL) was added at −78° C. The bath was removed and the reaction mixture was allowed to reach room temperature and stirring continued for 12 h. A saturated aqueous solution of ammonium chloride was added and extracted with ethyl acetate. Ethyl acetate layer was washed with water followed by brine solution. The organic layer was dried over sodium sulfate, filtered and concentrated. The crude was purified by silica gel chromatography (30-40% ethyl acetate in Hexane) to afford (9) as colorless gel. Yield (2.0 g, 65%); LCMS m/z 644.5 [M+1]⁺.

To a stirred solution of (9, 1.0 eq, 2.0 g, 3.11 mmol) in methanol (15 mL). was added Dowex-50W X8 (0.50 g) at room temperature under nitrogen atmosphere. The resulting mixture was stirred at room temperature for 2 h then filtered, washed with methanol and filtrate was concentrated under vacuum to afford (10) as off white solid. Yield: 1.10 g (83%); LC-MS; m/z, 426.47 [M−1].

To a stirred solution of (10, 1.00 eq, 0.89 g, 2.08 mmol) in pyridine (10 mL) was added an acetic anhydride (15.0 eq, 2.95 mL, 31.2 mmol) dropwise at 0° C. under nitrogen. The cold bath was removed and the resulting mixture was stirred at room temperature under nitrogen for 16 h. The volatiles were removed on a high vacuum and the residue was partitioned between ethyl acetate and aqueous 1N-HCl. The water layer was extracted again with ethyl acetate. The combined organic layers were dried over sodium sulfate, filtered, concentrated and purified by silica gel chromatography (30% ethyl acetate in dichloromethane) to afford (11) as thick syrup. Yield: 1.0 g (93%); LC-MS, m/z 554.54 [M+1]⁺.

To a stirred solution of (11, 1.00 eq, 1.0 g, 1.90 mmol) in tetrahydrofuran:ethyl acetate (1:1, 15 mL) 20% palladium hydroxide on carbon (0.50 g) and glacial acetic acid (1.5 eq, 0.162 mL, 2.83 mmol) were added at room temperature under nitrogen. The resulting mixture was stirred at room temperature under hydrogen gas pressure (10 psi) for 3 h. The reaction mixture filtered through celite bed and washed with methanol, filtrate concentrated under vacuum to afford (12) as brown sticky gel. Yield: 1.0 g (Crude); LCMS m/z 530.21 [M+1]⁺.

To a solution of (12, 1.0 eq, 1.00 g, 1.89 mmol) in NN-dimethyl formamide (7.0 mL), N,N-diisopropylethyl amine (1.0 eq, 0.20 mL, 1.19 mmol) and 4-nitrophenyl hex-5-yn-1-ylcarbamate (5.0 eq, 1.65 mL, 9.44 mmol) in N,N-dimethyl formamide (3.0 mL) were added. The reaction mixture was stirred at room temperature for 16 h. The reaction mixture was concentrated under reduced pressure to afford the crude. which was purified by reverse phase (Aq C-18 column) column chromatography using 20-50% acetonitrile in water as eluent. The fractions were washed with ethyl acetate. The organic layer dried over anhydrous sodium sulfate, filtered and concentrated under reduced pressure to afford (13) as brown sticky solid. Yield: 1.1 g (89%); LCMS m/z 653.21 [M+1]⁺.

To a stirred solution of (13, 1.0 eq, 1.0 g, 1.53 mmol) in dichloromethane (10.0 mL), pyridine (10.0 eq, 1.35 mL, 15.32 mmol) cooled to 0° C. and bromotrimethylsilane (10.0 eq, 1.68 mL, 15.32 mmol) was added and reaction mixture was stirred at room temperature for 16 h. After completion, reaction mixture was quenched with ice water, extracted with dichloromethane. The organic layer was dried, concentrated under reduced pressure to afford off white solid. It was further washed with diethyl ether and dried to afford (14) as off white solid. Yield: 0.87 g (95%); LCMS m/z 595.21 [M−1].

(14, 0.48 g, 1.0 eq, 0.816 mmol) dissolved in methanol (10.0 mL) and cooled to 0° C. followed by addition of sodium methoxide (0.18 mL, 1.0 eq, 0.816 mmol, 25% w/v solution in methanol). The reaction stirred at room temperature for 15 min and followed by TLC. After completion of reaction, Dowex-50wX8-Hydrogen form was added until a neutral pH was obtained. The reaction was filtered through sintered glass funnel, concentrated in vacuo and purified by reverse phase prep-HPLC purification with (30-45% acetonitrile in water with 0.1% TFA buffer) to get (2-((2R,3S,4R,5S,6R)-6-(4-(3-(hex-5-yn-1-yl)ureido)benzyl)-3,4,5-trihydroxytetrahydro-2H-pyran-2-yl)ethyl)phosphonic acid (I-101). Yield 0.015 g, (4%); LCMS, m/z 471.18 [M+1]+. H NMR (400 MHz, MeOD) δ 7.27 (d, J=8.4 Hz, 2H), 7.14 (d, J=8.4 Hz, 2H), 4.03 (t, J=8.4 Hz, 1H), 3.78-3.76 (m, 2H), 3.51-3.47 (m, 2H), 3.21 (t, J=6.8 Hz, 2H), 2.95-2.89 (m, 1H), 2.85-2.80 (m, 1H), 2.24-2.21 (m, 3H), 2.09-2.07 (m, 1H), 1.76-1.74 (m, 2H), 1.68-1.62 (m, 2H), 1.60-1.57 (m, 2H), 1.56-1.47 (m, 1H).

### Synthesis of Synthon Compound I-102

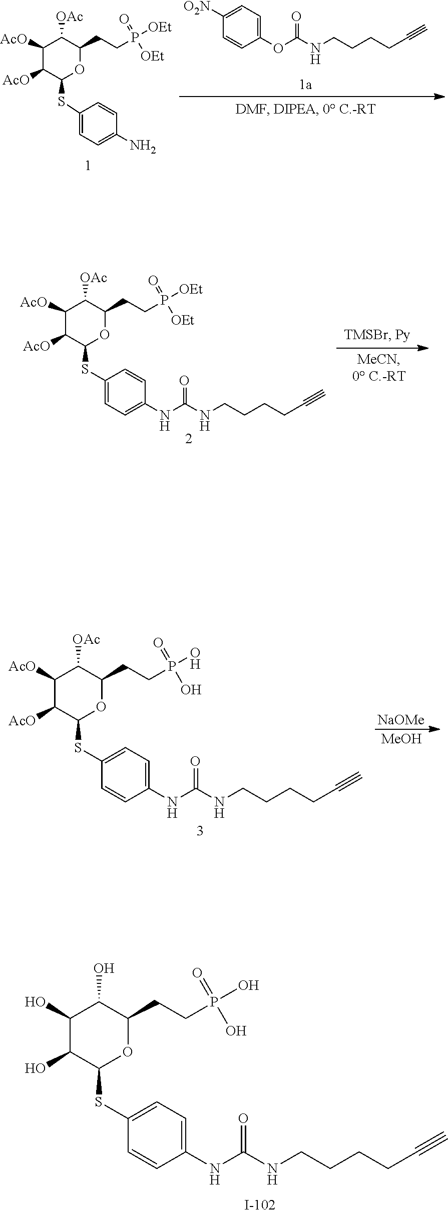

To a solution of (2S,3S,4S,5R,6R)-2-((4-aminophenyl)thio)-6-(2-(diethoxyphosphoryl)ethyl)tetrahydro-2H-pyran-3,4,5-triyl triacetate (1, 1.0 eq, 1.04 g, 1.90 mmol) in N,N-dimethyl formamide (12.0 mL), NN-diisopropylethyl amine (2.0 eq, 0.663 mL, 3.80 mmol) and 4-nitrophenyl hex-5-yn-1-ylcarbamate (1a, 2.0 eq, 0.996 g, 3.80 mmol) were added. The reaction mixture was stirred at room temperature for 16 h. The progress of reaction was monitored by LCMS. The reaction mixture was concentrated under reduced pressure to afford crude. The crude was purified by reverse phase (C-18 column) column chromatography using 20-50% acetonitrile in water as eluent. The fractions were washed with ethyl acetate. The organic layer dried over anhydrous sodium sulphate, filtered and concentrated under reduced pressure to afford (2) as brown sticky solid. Yield: 0.65 g (52.5%) LCMS m/z. 671.22 [M+1]⁺.

To a stirred solution of (2, 1.0 eq, 0.25 g, 0.373 mmol) in dichloromethane (8.0 mL), pyridine (10.0 eq, 0.30 mL, 3.73 mmol) cooled to 0° C. and bromotrimethylsilane (10.0 eq, 0.49 mL, 3.73 mmol) was added and reaction mixture was stirred at room temperature for 16 h. After completion, reaction mixture was quenched with ice water, extracted with dichloromethane. The organic layer separated, dried over anhydrous sodium sulfate, filtered and concentrated under reduced pressure to get off-white solid. It was further washed with diethyl ether and dried to afford (3) as off white solid. Yield: 0.16 g (69.8%) LCMS m/z. 614.93 [M+1]⁺.

To the stirred solution of (3, 1.0 eq, 0.08 g, 0.142 mmol) in methanol (3 mL), sodium methoxide 25% w/v in methanol (7.0 eq, 0.21 mL, 0.991 mmol) was added drop-wise to this solution and reaction mixture was allowed to stir at room temperature. The progress of the reaction was monitored by LCMS. After 2 h, reaction mixture was neutralized with Dowex-hydrogen form (200-400 mesh) (up to pH-7). The reaction mixture was filtered, concentrated under reduced pressure to get crude product. The crude was purified by prep-HPLC eluting from C18 column with 50-80% acetonitrile in water with 0.1% TFA. Fractions containing the desired product were combined and lyophilized to dryness to afford (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (I-102) as white solid. Yield: 0.016 g, 23.1%; LC-MS m/z. 489.17 [M+1]⁺. ¹H NMR (400 MHz, DMSO-d₆) δ 8.47 (s, 1H), 7.34 (d, J=8.8 Hz, 2H), 7.25 (d, J=8.4 Hz, 2H), 6.16 (t, J=5.6 Hz, 1H), 4.93 (bs, 1H), 4.78 (s, 2H), 3.81 (s, 1H), 3.31 (dd, J=3.2, 9.2 Hz, 1H), 3.22 (t, J=9.2 Hz, 1H), 3.08 (dd, J=6.0, 11.6 Hz, 2H), 3.02-2.97 (m, 1H), 2.77 (t, J=2.8 Hz, 1H), 2.20-2.16 (m, 2H), 2.07-1.99 (m, 1H), 1.78-1.67 (m, 1H). 1.54-1.41 (m, 6H).

### Synthesis of Synthon for X26

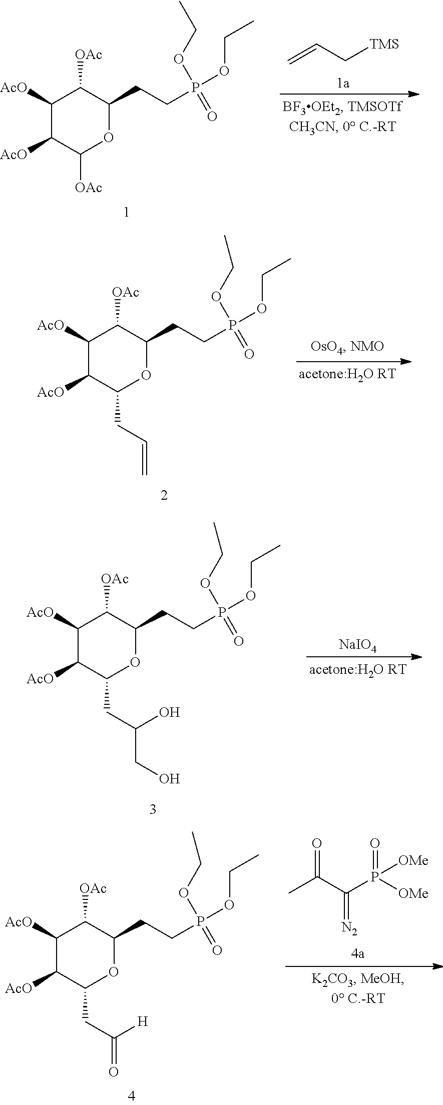

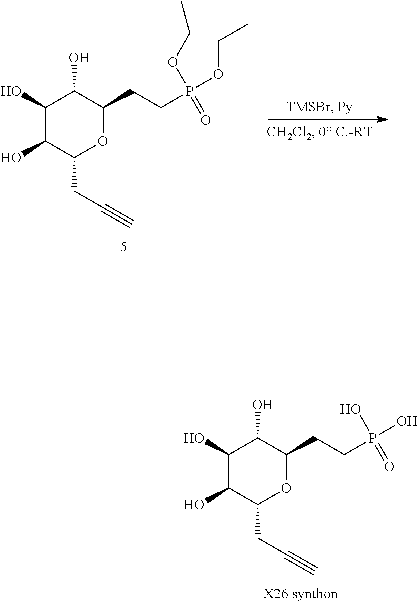 [SMILES: C#CC[C@H]1O[C@H](CCP(=O)(OCC)OCC)[C@@H](O)[C@H](O)[C@@H]1O ; "TMSBr, Py" ; C#CC[C@H]1O[C@H](CCP(=O)(O)O)[C@@H](O)[C@H](O)[C@@H]1O]

To a stirred solution of ((3S,4S,5R,6R)-6-(2-(diethoxyphosphoryl)ethyl)tetrahydro-2H-pyran-2,3,4,5-tetrayl tetraacetate (1, 1.0 eq., 4.3 g, 8.91 mmol) in acetonitrile (40 mL) were added allyltrimethylsilane (1a, 4.0 eq., 5.67 mL, 35.7 mmol) followed by boron trifluoride diethyl etherate (4.0 eq., 4.4 mL, 35.7 mmol) and trimethylsilyl trifluoromethanesulfonate (0.3 eq., 0.485 mL, 2.67 mmol) sequentially at 0° C. under nitrogen atmosphere. The reaction mixture was then stirred for 12 h at room temperature. After that, reaction mixture was poured into ice-cold saturated aqueous sodium bicarbonate solution and extracted with dichloromethane. Organic part was again washed with brine, dried over anhydrous sodium sulphate, concentrated and purified by silica gel column chromatography (using 10% methanol in dichloromethane) to give (2) as light yellow syrup. Yield: 3.48 g, 84.06%, LC-MS m/z 465.0 [M+1]⁺.

N-Methylmorpholine N-oxide (1.5 eq., 0.397 g, 1.5 eq, 3.39 mmol) followed by osmium tetraoxide (0.1 eq, 1.44 mL, 0.226 mmol, 4.0 wt % in water) were added to a stirred solution of (2R,3R,4R,5R,6R)-2-allyl-6-(2-(diethoxyphosphoryl)ethyl)tetrahydro-2H-pyran-3,4,5-triyl triacetate (2, 1.0 eq, 1.05 g, 2.26 mmol) in acetone-water (5:1, 30.0 mL) at room temperature. After 2 h, TLC showed complete consumption of starting material and a lower spot generated (based on TLC observation). Ethyl acetate (50 mL) was then added, and extracted with ethylacetate. The organic part was dried over anhydrous sodium sulfate, filtered and the solvent was removed in vacuo to get crude (3) which was directly used for next step.

To a stirred solution of crude (3, 1.2 g, 2.41 mmol) in a mixture of acetone: water (2:1, 20 mL) at 0° C., was added sodium periodate (2 eq, 1.03 g, 4.81 mmol) and then allowed to stir at room temperature. After being stirred at room temperature for 2 h, the TLC showed full consumption of starting material and a less polar new spot was generated on TLC. Then ethyl acetate was added to reaction mixture and extracted with ethyl acetate. The organic part was dried over anhydrous sodium sulfate, filtered and concentrated to give crude product which was then purified by flash column chromatography using 7-10% methanol in dichloromethane to give (4) as colorless syrup. Yield: 0.91 g, 81.04%. LC-MS m/z 467.1 [M+1]⁺.

To a solution of (4, 1.00 eq, 0.91 g, 1.95 mmol) in methanol (25.0 mL) at 0° C., were added potassium carbonate (3 eq., 0.809 g, 5.85 mmol), dimethyl (1-diazo-2-oxopropyl)phosphonate (4a, 2 eq., 0.75 g, 3.9 mmol) and reaction mixture was stirred at room temperature for 3 h. TLC showed formation of polar spot. The volatiles were then evaporated in vacuo to get crude reaction mass which was purified by silica gel flash column chromatography using 10-12% methanol in dichloromethane gave (5) as colorless syrup. Yield: 0.35 g, 53.34%. LC-MS m/z 337.0 [M+1]⁺.

To a stirred solution of (5, 1.0 eq, 0.35 g, 1.04 mmol) in dichloromethane (15.0 mL), were added pyridine (10.0 eq, 0.838 mL, 10.4 mmol) and bromotrimethylsilane (10.0 eq, 1.37 mL, 10.4 mmol) at 0° C. and reaction mixture was allowed to stir at room temperature. The progress of the reaction was monitored by LC-MS. After 16 h, volatiles were evaporated and the crude mass was purified by prep-HPLC (using 40-60% acetonitrile in water with 0.1% TFA). The fractions containing desired compound were collected and lyophilized to give (2-((2R,3S,4R,5S,6R)-3,4,5-trihydroxy-6-(prop-2-yn-1-yl)tetrahydro-2H-pyran-2 yl)ethyl)phosphonic acid (6) as off-white solid. Yield: 0.101 g, 34.64% LC-MS m/z 281.0 [M+1]⁺.

### Synthesis of Synthon Compound I-108

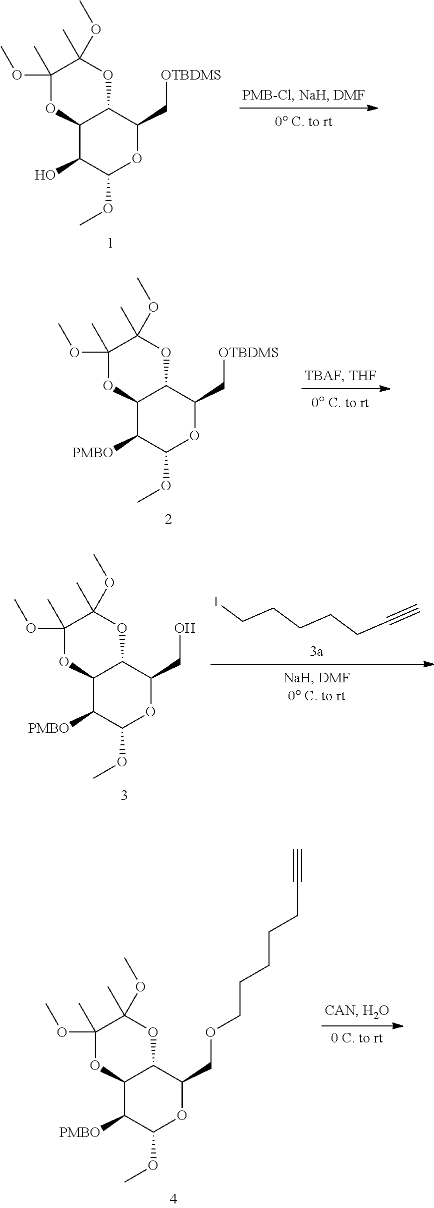

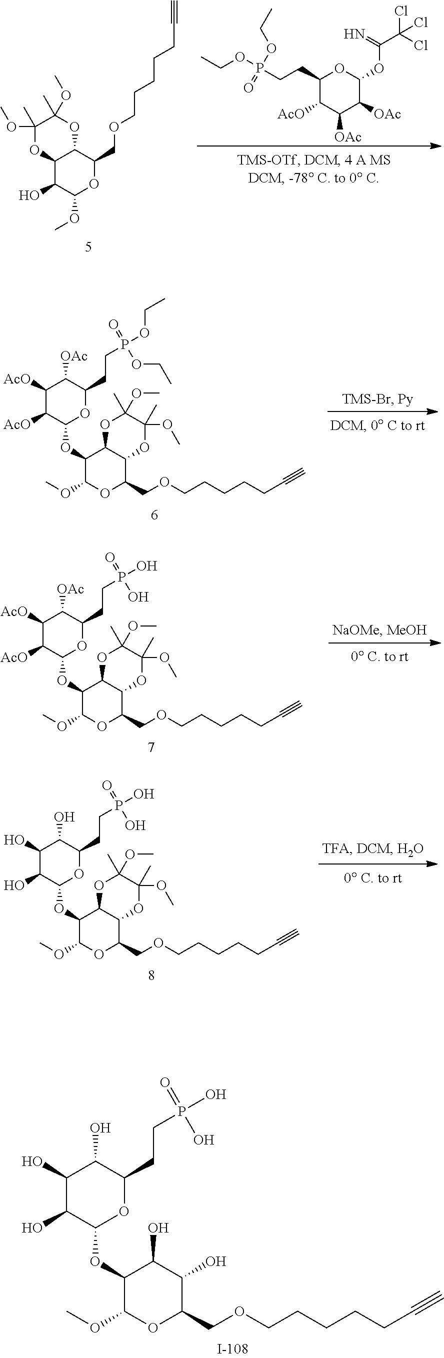

To a solution of (4aR,5R,7S,8S,8aR)-5-(((tert-butyldimethylsilyl)oxy)methyl)-2,3,7-trimethoxy-2,3-dimethylhexahydro-5H-pyrano[3,4-b][1,4]dioxin-8-ol (1, 0.90 g, 1 eq, 2.13 mmol) in N,N-dimethylformamide (10.0 mL) at 0° C. was added sodium hydride (0.170 g, 2 eq., 4.26 mmol) and p-methoxybenzylchloride (0.65 mL, 3.0 eq, 6.39 mmol) and tetrabutylammonium iodide (0.157 g, 0.2 eq., 0.426 mmol) and the reaction mixture stirred at 0° C. for 20 minutes. Reaction mixture was then partitioned in between ethyl acetate and water. Ethyl acetate layer was separated, washed with water, brine solution, and dried over anhydrous sodium sulfate and concentrated under reduced pressure to get crude product which was purified by combi-flash column chromatography (eluting with 10 to 20% ethyl acetate in hexane) to afford (2) as colorless sticky gum. Yield: 0.52 g (45%). LCMS: m/z 560.2 [M+18]⁺

To a solution of (2, 0.700 g, 1.0 eq, 1.33 mmol) in tetrahydrofuran (10 mL) at 0° C. was added tetrabutylammonium fluoride (1M solution in THF, 1.99 mL, 1.5 eq., 1.99 mmol) and reaction mixture was allowed to come slowly to room temperature and stirred for 4 h. Reaction mixture quenched by addition of cold water and extracted with ethyl acetate. Ethyl acetate layer was dried over anhydrous sodium sulfate and concentrated under reduced pressure to get crude product. which was purified by flash column chromatography using silica gel column and eluting with 30 to 40% ethyl acetate in hexane as eluent to afford (3) as colorless sticky gum. Yield: 0.300 g, 52.69%. LCMS: m/z 446.0 [M+18]⁺

To a solution of (3, 0.650 g, 1.0 eq, 1.52 mmol) in NN dimethylformamide (8.0 mL) at 0° C. was added sodium hydride (0.121 g, 2 eq., 3.03 mmol) and 7-iodohept-1-yne (3a, 0.674 g, 2 eq., 3.03 mmol) and reaction mixture was stirred at room temperature for 5 h. Reaction mixture was then partitioned in between ethyl acetate and water. Ethyl acetate layer was washed with water, brine, and the organic layer was dried over anhydrous sodium sulfate and concentrated under reduced pressure to get crude product which was purified by combi-flash column chromatography using silica gel column and eluting with 5 to 20% ethyl acetate in hexane as eluents to afford (4) as colorless sticky gum. Yield: 0.40 g (50.45%). LC-MS m/z 540.2 [M+18]⁺

To a solution of (4, 0.350 g, 1.0 eq, 0.67 mmol) in acetonitrile (6.00 mL) at 0° C., was added water (2.00 mL) and ceric ammonium nitrate (0.62 g, 1.5 eq, 1.14 mmol) and the reaction mixture was stirred at room temperature for 3 h. After that, reaction mixture was diluted with ethyl acetate and extracted with ethyl acetate. (4×40 mL). Ethyl acetate layer was dried over anhydrous sodium sulfate and concentrated under reduced pressure to obtain crude mass which was purified by column chromatography (eluting with 20 to 30% ethyl acetate in hexane as eluents) to obtain (5) as colorless sticky gum. Yield: 0.20 g (74.2%), LCMS: 420.0 [M+18]⁺

To a solution of (5, 0.370 g, 1.0 eq, 0.919 mmol) and (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (5a, 0.645 g, 1.2 eq., 1.10 mmol) in dry dichloromethane (24.0 mL) at 0° C., was added activated molecular sieves (0.300 g) and reaction mixture was stirred at 10° C. for 1 h. Reaction mixture was then cooled to −78° C. and trimethylsilyl trifluoromethanesulfonate (0.050 mL, 0.3 eq., 0.276 mmol) was added to reaction mixture and allowed to reach to 0° C. during 2 h. Thereafter, reaction mixture quenched by addition of triethylamine(0.129 mL, 1.0 eq, 0.919 mmol), and filtered to remove molecular sieves and reaction mixture was concentrated under reduced pressure to get crude product which was purified on combi-flash column chromatography (eluting with 20 to 50% ethyl acetate in dichloromethane as eluent) to afford (6) as pale yellow sticky gum. Yield: 0.55 g (72.53%). LCMS: m/z 842.3 (M+18)+

To a solution of (6, 0.630 g, 1.0 eq, 0.764 mmol) in dichloromethane (12.0 mL) at 0° C. were added pyridine (0.925 mL, 15 eq., 11.5 mmol), and bromotrimethylsilane (1.01 mL, 10 eq., 7.64 mmol) and reaction mixture was stirred at room temperature for 3 h. Reaction mixture cooled to 0° C. and quenched by addition of water (10 mL). Reaction mixture was extracted with dichloromethane (3×30 mL). The combined organic part was dried over anhydrous sodium sulfate and concentrated under reduced pressure to afford (7) as pale yellow sticky gum. Yield: 0.60 g (81.75%) LCMS: m/z 767.0 [M−1]⁻

To a solution of (7, 0.60 g, 1.0 eq, 0.780 mmol) in methanol (6.0 mL) at 0° C. was added sodium methoxide solution (0.515 mL, 3.0 eq, 2.34 mmol) and reaction mixture was stirred at room temperature for 2 h. After completion reaction mixture was cooled to 0° C. and neutralized with Dowex-50w X8-hydrogen form and filtered over sintered funnel, Filtrate obtained was concentrated under reduced pressure to afford (8) as pale yellow sticky gum. Yield: 0.37 g (73.77%) LC-MS m/z 641.0 (M−1)⁻

To a solution of (8, 0.37 g, 1.0 eq, 0.57 mmol) in dichloromethane (4.0 mL) at 0° C. was added trifluoroacetic acid (4.0 mL) and water (0.4 mL). Reaction mixture was then stirred at room temperature for 3 h. Reaction mixture concentrated under reduced pressure and re-dissolved in dichloromethane and concentrated again 3 times with dichloromethane to remove residual trifluoroacetic acid. Crude residue obtained was submitted directly for HPLC based Prep. Purification. Prep. Purification was done on HILIC column using 90 to 50% Acetonitrile in water and 0.1% trifluoro acetic acid. (Acetonitrile was used first) as buffer to afford (2-((2R,3S,4S,5S,6R)-6-(((2S,3S,4S,5S,6R)-6-((hept-6-yn-1-yloxy)methyl)-4,5-dihydroxy-2-methoxytetrahydro-2H-pyran-3-yl)oxy)-3,4,5-trihydroxytetrahydro-2H-pyran-2-yl)ethyl)phosphonic acid (I-108) as off white solid. Yield: 0.16 g (52.58%). LCMS: m/z 529.1 [M+1]⁺ ¹HNMR (400 MHz, D₂O) δ 5.04 (bs, 1H), 4.89 (bs, 1H), 4.07 (bs, 1H), 3.99 (bs, 1H), 3.89-3.81 (m, 3H), 3.75-3.52 (m, 8H), 3.44 (s, 3H), 2.36-2.35 (m, 1H), 2.25-2.21 (m, 2H), 2.16-2.09 (m, 1H), 1.99-1.88 (m, 1H), 1.76-1.70 (m, 2H), 1.67-1.60 (m, 2H), 1.58-1.50 (m, 2H).

### Synthesis of Synthon Compound I-109

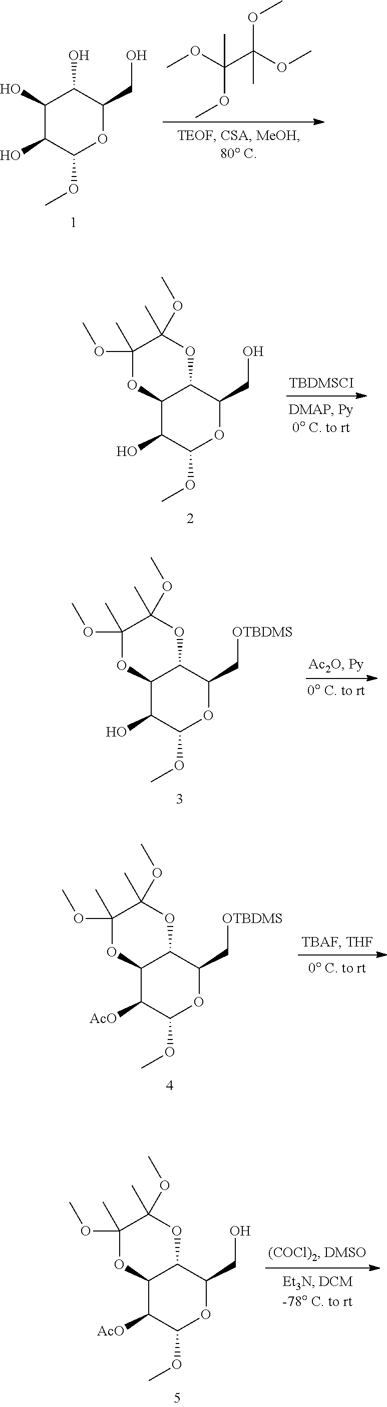

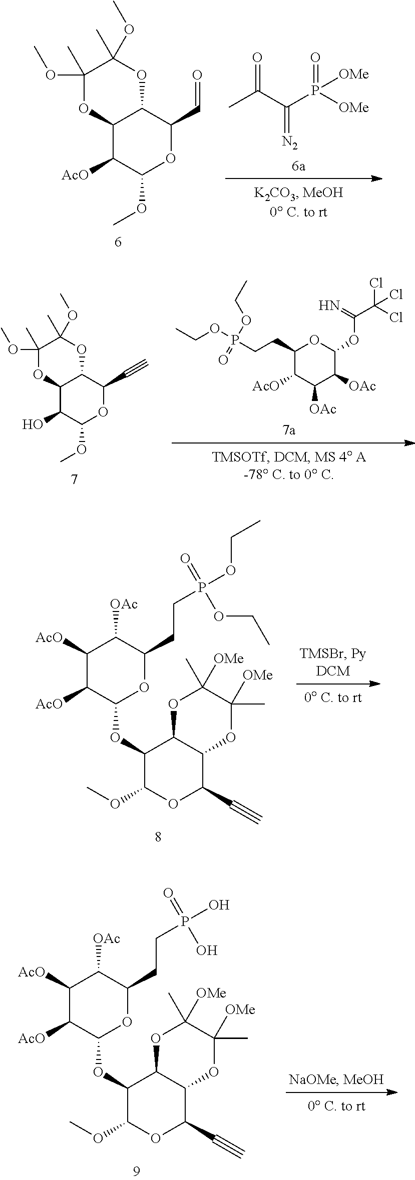

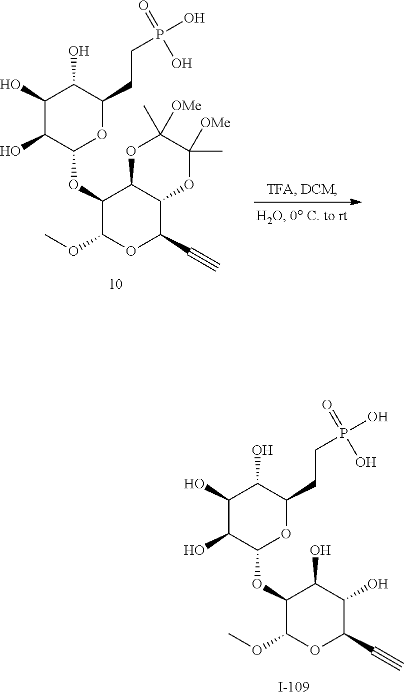

To a solution of methyl-α-D-mannopyranoside (6.00 g, 1.0 eq, 30.9 mmol) in methanol (100 mL) were added 2,2,3,3-tetramethoxybutane (1a, 5.51 g, 1.0 eq., 30.9 mmol), camphor sulfonic acid (0.431 g, 0.06 eq, 1.85 mmol) and trimethyl orthoformate (13.7 mL, 4.0 eq, 124 mmol). The solution was heated to reflux for 16 h. Full consumption of the starting material was indicated by TLC. The reaction was subsequently quenched with triethylamine (0.478 mL, 0.11 eq., 3.40 mmol) and concentrated under reduce pressure. The crude product was purified via column chromatography (20-50% Ethyl acetate in 0.1% triethylamine in dichloromethane) to afford (2) as a colorless thick syrup. Yield: 7.0 g (73.48%). LC-MS, m/z 326.0 [M+18]⁺.

To a solution of (2, 5.50 g, 1.0 eq., 17.8 mmol) in pyridine (50 mL) was added N,N-dimethylpyridin-4-amine (0.436 g, 0.2 eq., 3.57 mmol). Reaction mixture stirred for 5 min and cooled to 0° C. and tert-butyl(chloro)dimethylsilane (4.03 g, 1.5 eq., 26.8 mmol) was added portion-wise over 10 min. Reaction mixture was then stirred at room temperature for 16 h. Reaction mixture partitioned in between ethyl acetate and water. Organic layer was separated and aqueous layer reextracted with ethyl acetate. The combined organic layer was washed with water, brine solution, and dried over anhydrous sodium sulfate and concentrated under reduced pressure to get crude product. The crude product was purified by silica gel column chromatography eluting with 20 to 30% ethyl acetate in hexane. Desired fractions were concentrated under reduced pressure to afford (3) as a colorless oil. Yield: 4.80 g (63.68%). LC-MS, m/z 440.0 [M+18]⁺.

To a solution of (3, 4.93 g, 1.0 eq, 11.7 mmol) in pyridine (35.0 mL) at 0° C. was added acetic anhydride (2.21 mL, 2.0 eq., 23.3 mmol) and reaction mixture stirred at room temperature 3 h. Reaction mixture was then partitioned in between ethyl acetate and water. Ethyl acetate layer separated and aqueous layer re-extracted with ethyl acetate. The combined organic layer washed with water, brine solution, dried over anhydrous sodium sulfate and concentrated under reduced pressure to get crude product. The crude product was purified by silica gel column chromatography eluting with 0 to 20% ethyl acetate in hexane. Desired fractions were concentrated under reduced pressure to afford (4) as a colorless oil. Yield: 2.90 g, (53.5%). LC-MS, m/z 482.0 [M+18]⁺.

To a solution of (4, 2.10 g, 1.0 eq, 4.52 mmol) in tetrahydrofuran (40 mL) at 0° C. was added tetrabutylammonium fluoride (1M solution in THF, 5.42 mL, 1.2 eq., 5.42 mmol) and reaction mixture was allowed to come slowly at room temperature and stirred for 4 h. Reaction mixture was then quenched by addition of cold water and extracted with ethyl acetate. Ethyl acetate layer was dried over anhydrous sodium sulfate and concentrated under reduced pressure to get crude product which was then purified by flash column chromatography eluting with 30 to 40% ethyl acetate in hexane with 0.10% triethylamine. (Note: Column was neutralized with 0.10% triethylamine in hexane and 0.1% triethyl amine in hexane was used) as eluents to afford (5) as colorless sticky gum. Yield: 1.50 g (94.72%). LC-MS, m/z 368.0 [M+18]⁺.

To a solution of oxalic dichloride (0.45 mL, 1.1 eq., 5.34 mmol) in dry dichloromethane (15.0 mL) at −78° C., was added dimethyl sulfoxide (0.750 mL, 2.2 eq., 10.7 mmol) and the solution was stirred for 20 min at −78° C. To this solution (5, 1.70 g, 1.0 eq, 4.85 mmol) in dichloromethane (15.0 mL) added dropwise over 5 min. Reaction mixture was then stirred at −78° C. for 2 h and then triethylamine (3.41 mL, 5 eq., 24.3 mmol) was added dropwise. Reaction mixture was then stirred for 10 min at −78° C. and then at room temperature for another 2 h. Reaction mixture was partitioned in between ethyl acetate and water. Ethyl acetate layer separated and dried over anhydrous sodium sulfate and concentrated under reduced pressure to afford (6) as pale yellow sticky gum. Yield: 1.60 g (85.2%). LC-MS, m/z 349.0 [M+1]⁺.

To a solution of (6, 1.60 g, 1.0 eq, 4.59 mmol) in methanol (24.0 mL) at 0° C. was added potassium carbonate (1.90 g, 3.0 eq., 13.8 mmol) and dimethyl (1-diazo-2-oxopropyl)phosphonate (1.76 g, 2.0 eq., 9.19 mmol) and reaction mixture was stirred at room temperature for 3 h. TLC showed formation of non-polar spot. Reaction mixture partitioned in between ethyl acetate and water. Aqueous layer reextracted with ethyl acetate. Combined ethyl acetate layer was dried over anhydrous sodium sulfate and concentrated to get crude product. which was purified by combi-flash column chromatography (eluting with 20 to 30% ethyl acetate in hexane) to afford (7) as off white solid. Yield: 0.85 g, (61.21%). LC-MS, m/z 320.0 [M+18]⁺.

To a solution of (7, 1.20 g, 1.00 eq, 3.97 mmol) and (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (7a, 3.02 g, 1.3 eq., 5.16 mmol) in dry dichloromethane (72.0 mL) was added activated molecular sieves (0.50 g) and reaction mixture stirred at room temperature for 30 minutes. Reaction mixture was then cooled to −78° C. and trimethylsilyl trifluoromethanesulfonate (0.217 mL, 0.3 eq., 1.19 mmol) was added to reaction mixture and reaction mixture was allowed to reach to 0° C. during 2 h. Reaction mixture was then quenched by addition of triethyl amine (0.558 mL, 1.0 eq, 3.97 mmol) and reaction mixture concentrated under reduced pressure and purified by flash column chromatography (Note: Silica gel column was neutralized with 0.1% triethyl amine in dichloromethane) (eluting with 0 to 50% ethyl acetate in dichloromethane with 0.1% triethyl amine as eluents) to afford (8) as pale yellow solid. Yield: 1.60 g (55.62%). LC-MS m/z 742.0 [M+18]⁺

To a solution of (8, 1.60 g, 1.00 eq, 2.21 mmol) in dichloromethane (40.0 mL) at 0° C. were added pyridine (2.67 mL, 15 eq., 33.1 mmol), and bromotrimethylsilane (2.91 mL, 10 eq., 22.1 mmol) and the reaction mixture was stirred at room temperature for 3 h. Reaction mixture was then cooled to 0° C. and quenched by addition of water (10 mL). Reaction mixture was extracted with dichloromethane. Organic layer was dried over anhydrous sodium sulfate and concentrated under reduced pressure to afford (9) as off white solid. Yield: 1.30 g (79.20%). LCMS m/z 667.1 [M−1]⁻

To a solution of (9, 1.30 g, 1.0 eq, 1.94 mmol) in Methanol (25.0 mL) at 0° C. was added sodium methoxide solution (25% in Methanol, 1.26 mL, 3.0 eq, 5.83 mmol) and reaction mixture was stirred at room temperature for 2 h. After completion, reaction mixture was cooled to 0° C. and neutralized with Dowex-50w X8 hydrogen form and filtered over sintered funnel, Filtrate obtained was concentrated under reduced pressure to afford (10) as pale yellow sticky gum. Yield: 1.20 g (96.7%).541.1 [M−1]⁻

To a solution of (10, 1.20 g, 1.0 eq, 2.21 mmol) in dichloromethane (6.0 mL) at 0° C. was added trifluoroacetic acid (6.0 mL) and water (0.6 mL). Reaction mixture was then stirred at room temperature for 3 h. Thereafter, reaction mixture was concentrated under reduced pressure and dissolved in dichloromethane and concentrated again 3 times with dichloromethane to remove residual trifluoroacetic acid. Crude residue obtained was submitted directly for Prep. HPLC purification. Purification was done on HILIC column using 90 to 50% acetonitrile in water and 0.1% acetic acid (Acetonitrile was used first) as buffer to afford (2-((2R,3S,4S,5S,6R)-6-(((2S,3S,4S,5S,6R)-6-ethynyl-4,5-dihydroxy-2-methoxytetrahydro-2H-pyran-3-yl)oxy)-3,4,5-trihydroxytetrahydro-2H-pyran-2-yl)ethyl)phosphonic acid (I-109) as off white solid. Yield:0.50 g (52.77%). LC-MS m/z 429.0 [M+1]. ¹H NMR (400 MHz, D₂O) δ 5.03 (d, J=1.6 Hz, 1H), 4.91 (d, J=1.2 Hz, 1H), 4.33 (dd, J=9.2, 1.6 Hz, 1H), 4.08-4.07 (m, 1H), 3.87 (s, 1H), 3.87-3.76 (m, 3H), 3.67-3.63 (m, 1H), 3.57-3.52 (m, 1H), 3.46 (s, 3H), 2.98 (d, J=2.0 Hz, 1H), 2.16-2.07 (m, 1H), 1.96-1.85 (m, 1H), 1.82-1.73 (m, 2H).

### Synthesis of Synthon Compound 110A

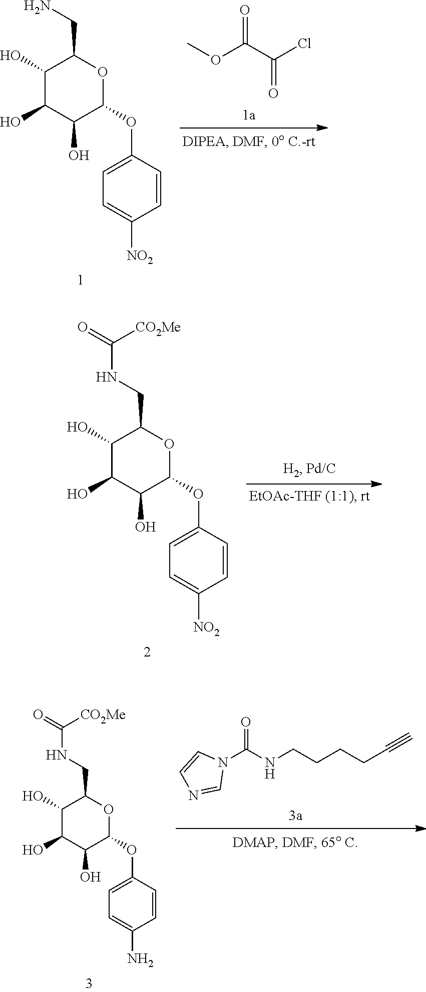

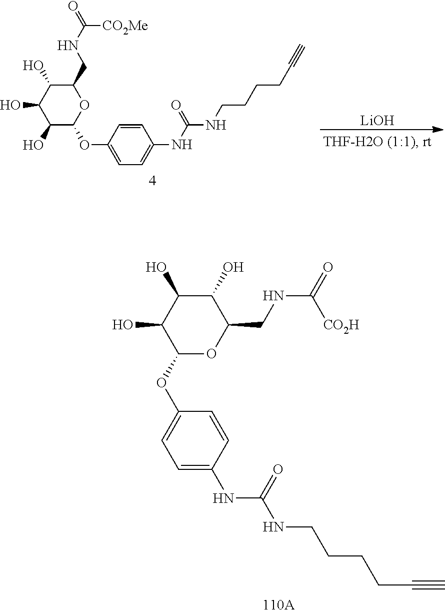 [SMILES: C#CCCCCNC(=O)Nc1ccc(O[C@H]2O[C@H](CNC(=O)C(=O)OC)[C@@H](O)[C@H](O)[C@@H]2O)cc1 ; "LiOH" ; C#CCCCCNC(=O)Nc1ccc(O[C@H]2O[C@H](CNC(=O)C(=O)O)[C@@H](O)[C@H](O)[C@@H]2O)cc1]

To a solution of (2R,3S,4S,5S,6R)-2-(aminomethyl)-6-(4-nitrophenoxy)tetrahydro-2H-pyran-3,4,5-triol (1, 1.0 eq., 0.400 g, 1.33 mmol) in anhydrous N,N-dimethylformamide (2.00 mL), N,N-diisopropylethyl amine (3.0 eq., 0.696 mL, 3.99 mmol) and methyl 2-chloro-2-oxoacetate (1.5 eq., 0.184 mL, 2.00 mmol) were sequentially added at 0° C. and the reaction mixture was stirred at the same temperature for 2 h. After which TLC showed complete conversion of starting material. The volatiles were evaporated under reduced pressure and the crude residue was purified by flash column chromatography (using 2-5% methanol in dichloromethane) to afford (2) as a colorless viscous liquid. Yield: 0.450 g, 45.47%. LCMS; m/z 387.2 [M+1]⁺.

To a solution of (2, 1.0 eq., 0.450 g, 1.16 mmol) in ethyl acetate:tetrahydrofuran (1:1) (10.0 mL), was added 10% palladium on carbon (0.225 g). The reaction mixture was stirred at room temperature for 3 h under hydrogen atmosphere. After completion (monitored by LCMS), the reaction mixture was passed through celite bed using sintered funnel. The filtrate was concentrated under reduced pressure to afford (3) as colorless viscous liquid which was directly used in the next step without further purification. Yield: 0.230 g (Crude); LCMS; m/z 357.0 [M+1]⁺.

To a solution of methyl 2-((((2R,3S,4S,5S,6R)-6-(4-aminophenoxy)-3,4,5-trihydroxytetrahydro-2H-pyran-2-yl)methyl)amino)-2-oxoacetate (1.0 eq., 0.230 g, 0.645 mmol) and N-(hex-5-yn-1-yl)-1H-imidazole-1-carboxamide (3a, 1.5 eq., 0.185 g, 0.968 mmol) in N,N-dimethylformamide (2.00 mL), was added NN-dimethylpyridin-4-amine (1.0 eq., 0.078 g, 0.645 mmol) at room temperature. The reaction mixture was then heated at 65° C. for 16 h. After completion, the reaction mixture was concentrated under vacuum to get crude (4) as a deep yellow syrup which was directly used in the next step without further purification. Yield: 0.250 g (Crude); LCMS; m/z 480.0 [M+1]⁺.

To a solution of (4, 1.0 eq., 0.250 g, 0.521 mmol) in tetrahydrofuran:water (1:1) (4.00 mL), was added lithium hydroxide (3.0 eq., 0.037 g, 1.56 mmol) at room temperature and the reaction mixture was stirred at the same temperature for 12 h, after which TLC showed complete consumption of starting material. The reaction mixture was concentrated under vacuum to get crude residue which was directly used for prep. HPLC purification (using 15-35% acetonitrile in water with 0.1% TFA). All the fractions containing desired compound were combined and lyophilized to afford 2-((((2R,3S,4S,5S,6R)-6-(4-(3-(hex-5-yn-1-yl)ureido)phenoxy)-3,4,5-trihydroxytetrahydro-2H-pyran-2-yl)methyl)amino)-2-oxoacetic acid (110A) as off-white solid. Yield: 0.018 g, 7.42%. LCMS; m/z 466.3 [M+1]⁺

### Synthesis of Synthon Compound 111A

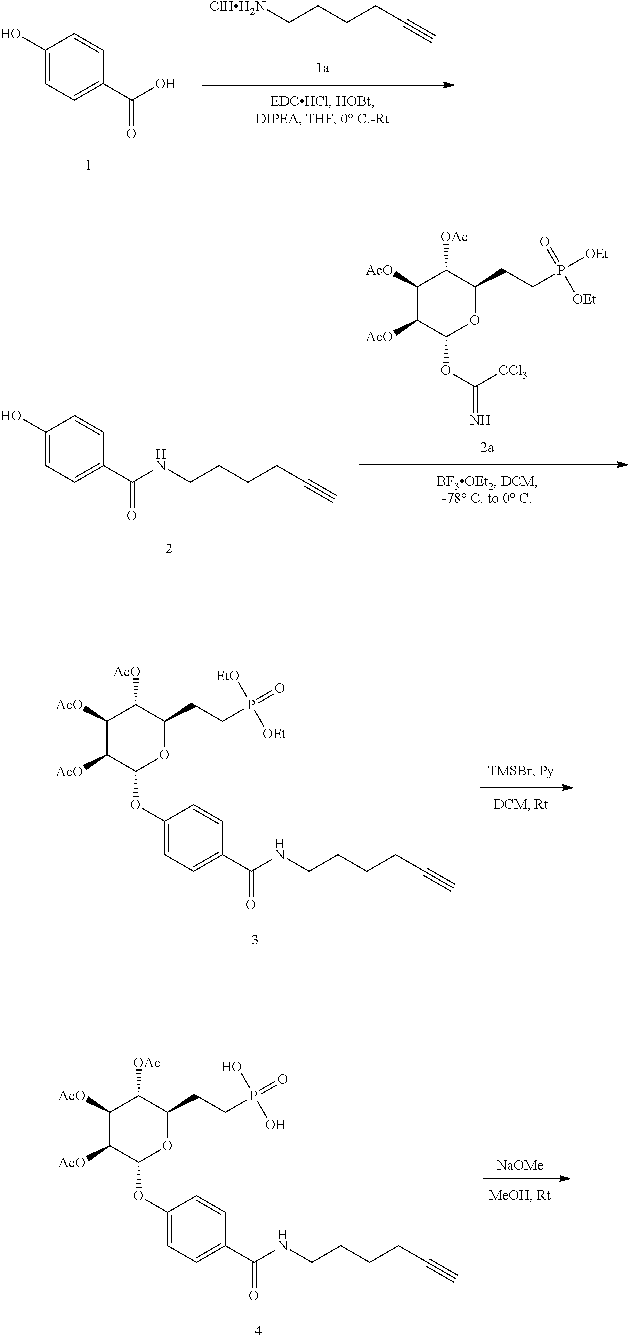

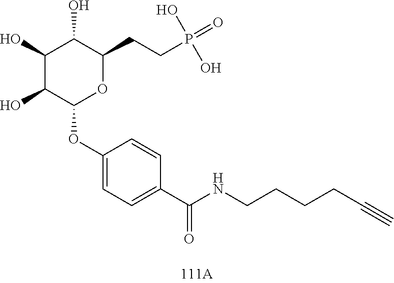 [SMILES: C#CCCCCNC(=O)c1ccc(O[C@H]2O[C@H](CCP(=O)(O)O)[C@@H](O)[C@H](O)[C@@H]2O)cc1]

A solution of 4-hydroxybenzoic acid (0.6 g, 1.0 eq, 4.34 mmol) and hex-5-yn-1-amine hydrochloride (1a, 1.1 eq, 0.638 g, 4.78 mmol) in tetrahydrofuran (10 mL) was cooled to 0° C. N,N-diisopropylethylamine (4.01 mL, 5.0 eq, 21.7 mmol), ({[3-(dimethylamino)propyl]imino}methylidene)(ethyl)amine hydrochloride (EDC·HCl)(1.25 g, 1.5 eq, 6.52 mmol), and 1H-1,2,3-benzotriazol-1-ol (HOBt) (0.88 g, 1.5 eq, 6.52 mmol) were added and the reaction mixture was stirred at room temperature for 16 h. After completion, reaction mixture was concentrated to get crude which was purified by flash column chromatography (using 70% ethyl acetate in dichloromethane) to afford N-(hex-5-yn-1-yl)-4-hydroxybenzamide (2) as brown syrup. Yield: 0.780 g, 75.59%. LCMS, m/z 216.04 [M−1]⁻.

To a stirred solution of (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (1.0 g, 1.0 eq, 1.71 mmol) and N-(hex-5-yn-1-yl)-4-hydroxybenzamide (2, 0.446 g, 1.2 eq., 2.05 mmol) in dry dichloromethane (20.0 mL) at −78° C., was added boron trifluoride diethyl etherate (0.422 mL, 2.0 eq., 3.42 mmol). After 5 minutes, −78° C. cold bath was removed and replaced with a 0° C. cold bath. The reaction mixture was stirred at 0° C. for 2 h. Thereafter, reaction mixture was partitioned between dichloromethane and saturated aqueous sodium bicarbonate. The water layer was extracted again with dichloromethane. The combined organics were dried over sodium sulfate, filtered, concentrated on a rotary evaporator to give crude mass which was purified by flash silica gel column chromatography (using 80-85% ethyl acetate in dichloromethane) to give (3) as colorless syrup. Yield: 1.0 g, 91.42%, LCMS, m/z 640.35 [M+1]⁺.

To a solution of (3, 1.0 eq, 0.5 g, 0.782 mmol) in dichloromethane (5.0 mL), pyridine (15.0 eq, 0.966 mL, 11.7 mmol) was added. Then bromotrimethylsilane (10.0 eq, 1.03 mL, 7.82 mmol) was added at 0° C. and the reaction mixture was stirred at room temperature for 12 h. After completion (monitored by LCMS), the reaction mixture was diluted with water and extracted with dichloromethane. The organic layer was dried over sodium sulfate, filtered and concentrated under reduced pressure to obtain (4) as crude which was used for next reaction without further purification. Yield: 0.538 g (crude, 80% LCMS purity). LCMS, m/z 582.23 [M−1]⁻.

To a stirred solution of (4, 0.538 g, 1.0 eq, 0.922 mmol) in methanol (5 mL), was added 25% solution of sodium methoxide (0.609 mL, 3.0 eq, 2.77 mmol) at 0° C. and then stirred at room temperature. The progress of the reaction was monitored by LC-MS. After 2 h, the reaction was neutralized by adding Dowex-50, hydrogen form to neutral pH. Thereafter, the resin was filtered through sintered funnel and the filtrate was evaporated to give crude mass which was then purified by prep. HPLC (using 30-45% acetonitrile in water with 0.1% TFA.) to give (2-((2R,3S,4S,5S,6R)-6-(4-(hex-5-yn-1-ylcarbamoyl)phenoxy)-3,4,5-trihydroxytetrahydro-2H-pyran-2-yl)ethyl)phosphonic acid (111A) as off white solid. Yield: 0.139 g, 32.96%. LCMS, m/z 456.18 [M−1]⁻.

### Synthesis of Synthon Compound 112A

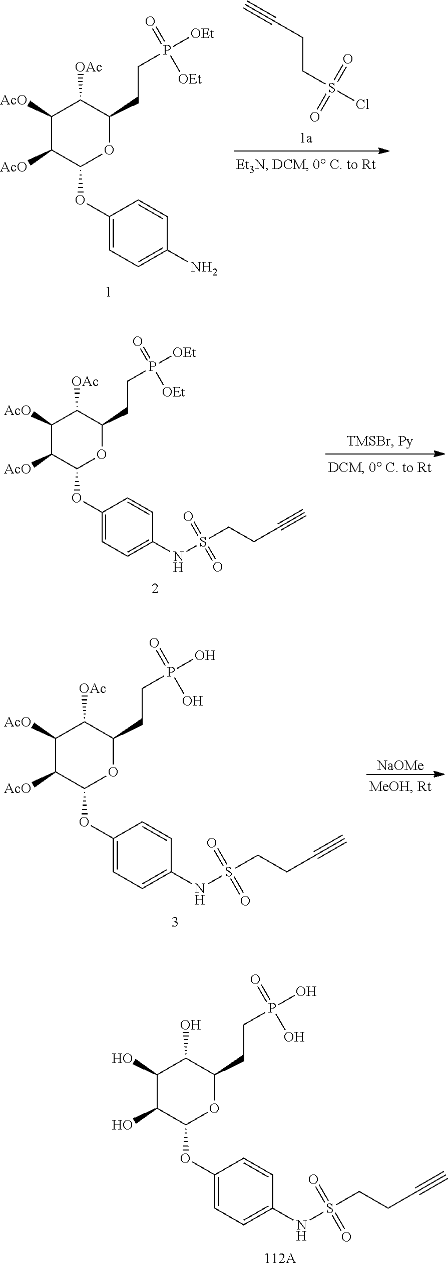

To a stirred solution of (2R,3S,4S,5R,6R)-2-(4-aminophenoxy)-6-(2-(diethoxyphosphoryl)ethyl)tetrahydro-2H-pyran-3,4,5-triyl triacetate (1, 1.0 eq, 0.500 g, 0.941 mmol) in dichloromethane (5.0 mL), were added but-3-yne-1-sulfonyl chloride (1a, 2.0 eq, 0.287 g, 1.88 mmol) and triethylamine (2.5 eq, 0.331 mL, 2.35 mmol) at 0° C. The reaction mixture was then stirred at room temperature for 12 h. After completion, the reaction mixture was poured into water and extracted with dichloromethane. The organic layer was dried over sodium sulfate, filtered and concentrated under reduced pressure to obtain crude which was purified by flash chromatography (silica mesh: 100-200; elution: 30-40% ethyl acetate in dichloromethane) to afford (2) as yellow syrup. Yield: 0.270 g, 41.0%. LCMS, m/z 646.0 [M−1]⁻.

To a solution of (2, 1.0 eq, 0.270 g, 0.417 mmol) in dichloromethane (3.0 mL), pyridine (15.0 eq, 0.515 mL, 6.25 mmol) was added. Then bromotrimethylsilane (10.0 eq, 0.550 mL, 4.17 mmol) was added at 0° C. and the reaction mixture was stirred at room temperature for 12 h. After completion, the reaction mixture was poured into water and extracted with dichloromethane. The organic layer was dried over sodium sulfate, filtered and concentrated under reduced pressure to obtain crude (3) as yellow syrup which was used for next reaction without further purification. Yield: 0.160 g (crude, 58% LCMS purity). LCMS, m/z 590.4 [M−1]⁻.

To a stirred solution of (3, 0.070 g, 1.0 eq, 0.118 mmol) in methanol (1.0 mL), was added 25% solution of sodium methoxide (0.025 mL, 0.2 eq, 0.0237 mmol) at 0° C. and then stirred at room temperature. The progress of the reaction was monitored by LCMS. After 2 h, the reaction was neutralized by adding Dowex-50, hydrogen form to neutral pH. Thereafter, the resin was filtered through sintered funnel and the filtrate was evaporated to give crude mass which was then purified by prep. HPLC (using 5-23% acetonitrile in water with 0.1% TFA.) to give (2-((2R,3S,4S,5S,6R)-6-(4-(but-3-yn-1-ylsulfonamido)phenoxy)-3,4,5-trihydroxytetrahydro-2H-pyran-2-yl)ethyl)phosphonic acid (112A) as off white solid. Yield: 0.028 g, 50.84%. LCMS, m/z 466.0 [M+1]⁺.

### Synthesis of Synthon 113A

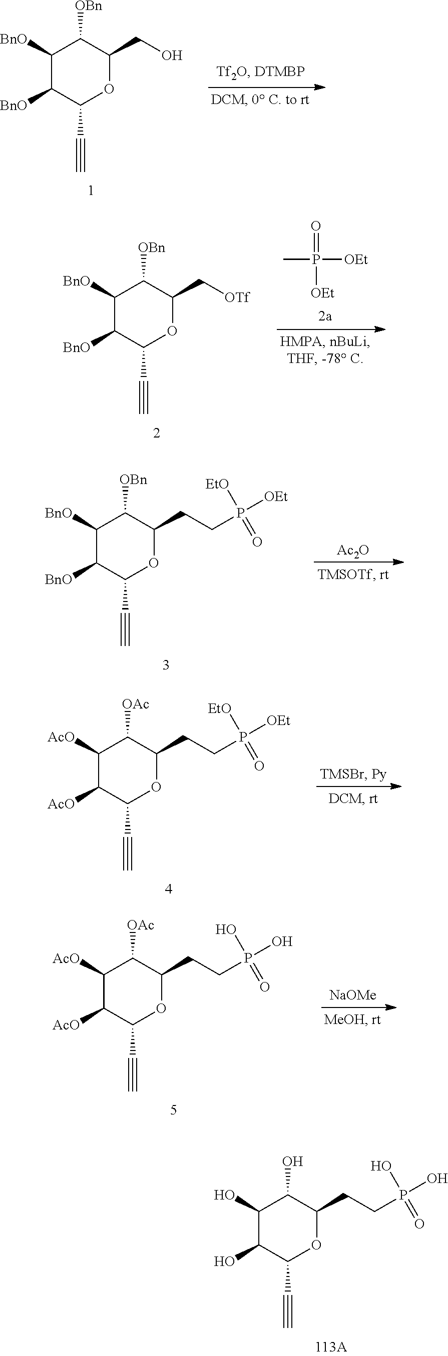

To a stirred solution of ((2R,3R,4R,5R,6R)-3,4,5-tris(benzyloxy)-6-ethynyltetrahydro-2H-pyran-2-yl)methanol (3.88 g, 1.0 eq, 8.46 mmol) in dichloromethane (40 mL) were added 2,6-di-tert-butyl-4-methylpyridine (2.95 g, 1.5 eq, 14.4 mmol) and trifluoromethanesulfonyl trifluoromethanesulfonate (1.99 mL, 1.2 eq, 11.8 mmol) at −40° C. and allowed to stirred at the same temperature. The progress of the reaction was monitored by TLC. After being stirred for 2 h, TLC showed a full consumption of starting material and a new non-polar spot was generated. Then the reaction mixture was evaporated under reduced pressure and the crude (2) obtained was directly used for next reaction immediately.

To a stirred solution of diethyl methylphosphonate (2a, 6.19 mL, 5.0 eq., 42.3 mmol) in tetrahydrofuran (25.00 mL) were added hexamethylphosphoramide (7.36 mL, 5 eq., 42.3 mmol) and n-butyllithium (16.3 mL, 4.8 eq., 40.6 mmol, 2.5 M in hexane) sequentially at −78° C. After 1 h, a solution of crude [(2R,3R,4R,5R,6R)-3,4,5-tris(benzyloxy)-6-ethynyloxan-2-yl]methyl trifluoromethanesulfonate (crude, obtained from step-1) in tetrahydrofuran (25.0 mL) was added to the reaction mixture and allowed to stir at −78° C. After 1 h, the reaction was quenched with saturated ammonium chloride solution and extracted with ethyl acetate. Organic layer was dried over anhydrous sodium sulphate, filtered and evaporate to afford the crude which was purified by flash column chromatography (using 30-35% ethyl acetate in hexane) to afford (3) as colorless liquid. Yield: 3.2 g, 63.78%; LCMS, m/z 593.34 [M+1]⁺.

(3, 1.50 g, 1.0 eq., 2.53 mmol) was dissolved in acetic anhydride (60 mL) and then cooled to 0° C. before trimethylsilyl trifluoromethanesulfonate (3.66 mL, 8.0 eq., 20.2 mmol) was added under nitrogen atmosphere. The reaction was then warmed to room temperature and stirred for 72 h. Dark brown solution was then cooled to 0° C. and quenched by the cautious addition of a saturated solution of sodium bicarbonate. The reaction mixture was diluted with ethyl acetate followed by washing of the organic layer with a saturated solution of sodium bicarbonate, water, and brine. Organic layer was dried over anhydrous sodium sulphate, filtered and evaporated to provide a dark brown oil which was purified by flash column chromatography (using 35-40% ethyl acetate in hexane) to yield (4) as brown syrup. Yield: 1.0 g, 88.12%. LC-MS, m/z 448.80 [M+1]⁺.

To a stirred solution of (4, 1.5 g, 1.0 eq., 3.35 mmol) in dichloromethane (25.0 mL) were added pyridine (2.69 mL, 10.0 eq., 33.5 mmol) and bromotrimethylsilane (4.41 mL, 10.0 eq., 33.5 mmol) sequentially at 0° C. and allowed to stir at room temperature. The progress of the reaction was monitored by LC-MS. After 16 h, water was added to reaction mixture, and extracted with dichloromethane. The organic part was dried over sodium sulphate and concentrated to dryness. The obtained was washed with diethyl ether several time to give (5). Yield: 1.1 g, 83.8%. LC-MS, m/z 390.60 [M−1]⁻

To a stirred solution of (5, 1.3 g, 1 eq, 3.31 mmol) in methanol (20 mL), was added 25% solution of sodium methoxide (2.19 mL, 3.0 eq, 9.94 mmol) at 0° C. and then stirred at room temperature. The progress of the reaction was monitored by LC-MS. After 2 h, the reaction was neutralized by adding Dowex-50, hydrogen form. Thereafter, the resin was filtered through sintered funnel and the filtrate was evaporated to give crude mass which was then purified by prep. HPLC (using 40-60% acetonitrile in water with 0.1% TFA) to give (2-((2R,3S,4S,5S,6R)-6-ethynyl-3,4,5-trihydroxytetrahydro-2H-pyran-2-yl)ethyl)phosphonic acid (113A) as off white solid. Yield: 0.175 g, 19.84%. LC-MS m/z 267.0 [M+1]⁺.

### Synthesis of Synthon 566A

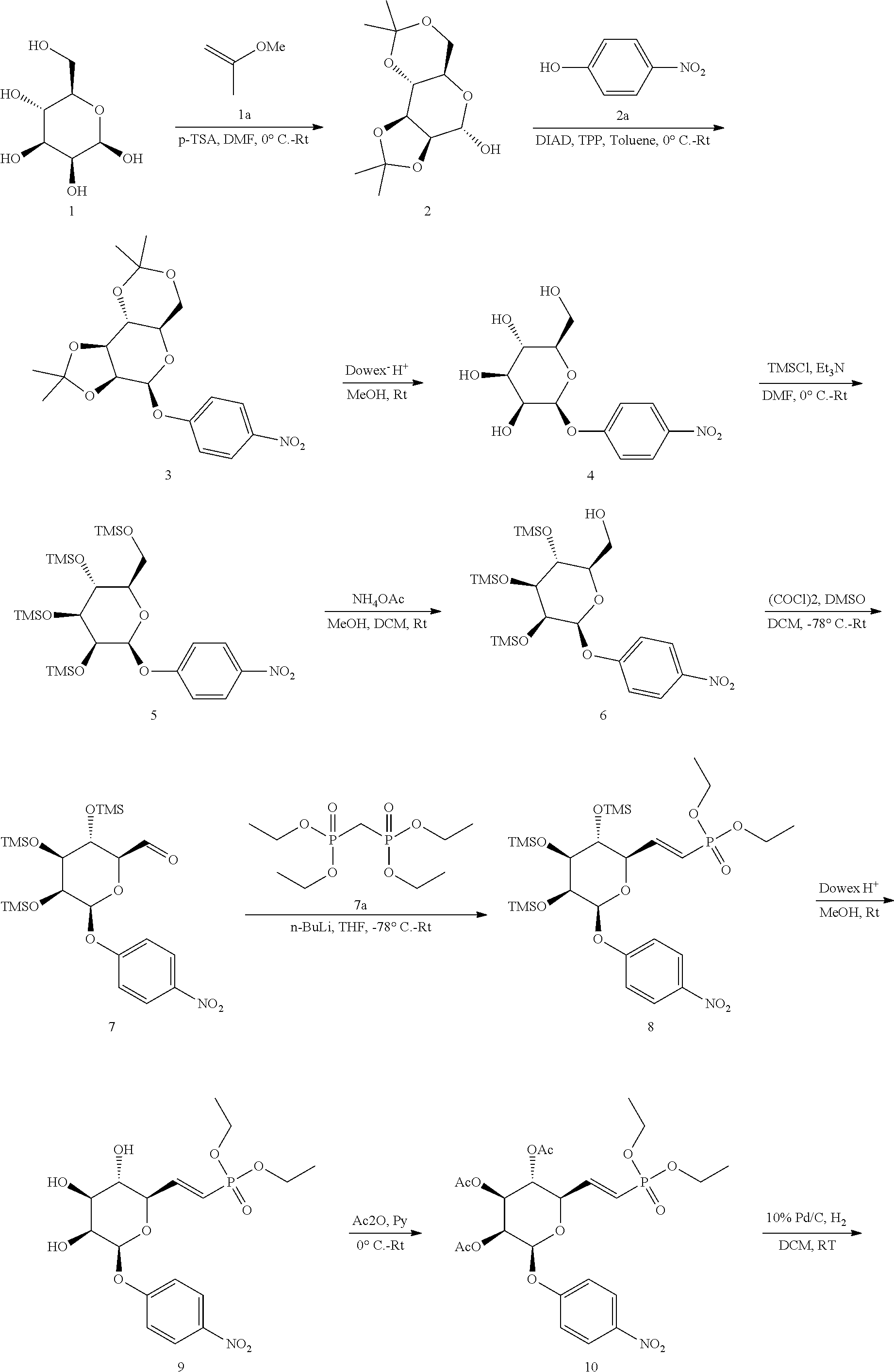

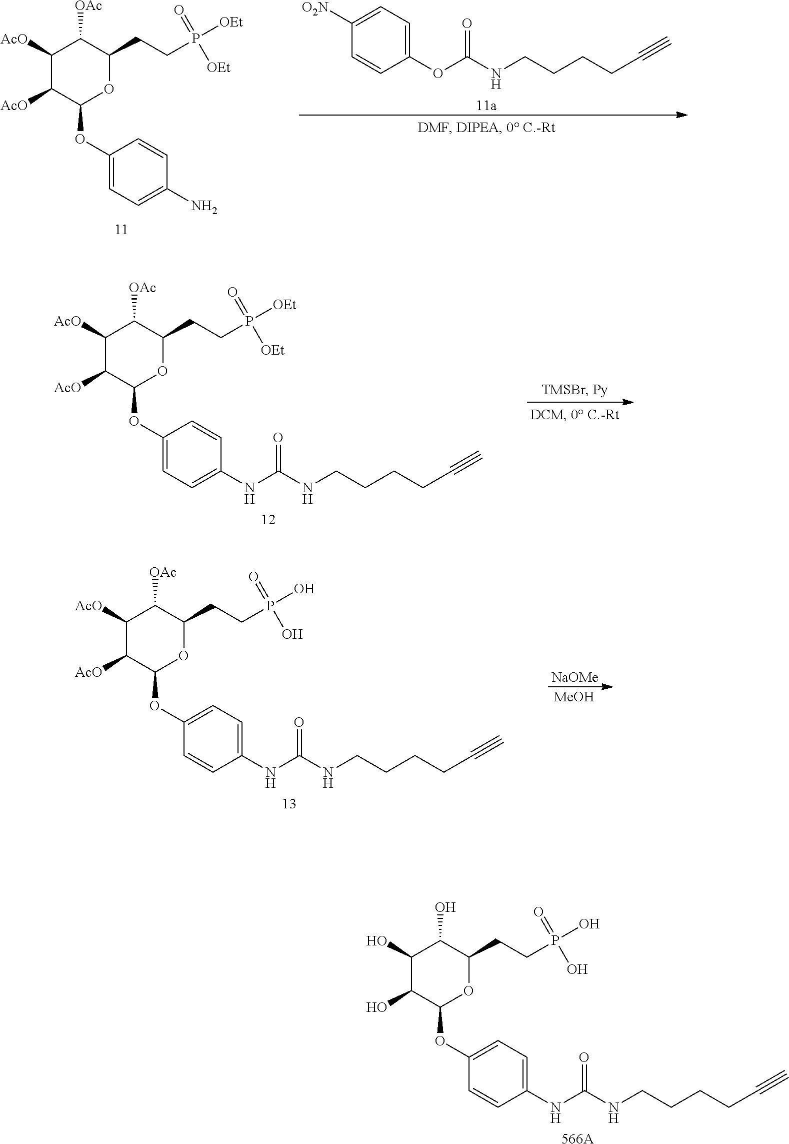

To a stirred solution of (2S,3S,4S,5S,6R)-6-(hydroxymethyl)tetrahydro-2H-pyran-2,3,4,5-tetraol (40.0 g, 1.0 eq, 222.0 mmol) in N,N-dimethyl formamide (150.0 mL) at 0° C., p-toluenesulfonic acid monohydrate (1.91 g, 0.05 eq, 11.1 mmol) was added in portion wise, followed by 2-methoxyprop-1-ene (77.7 mL, 5.0 eq, 1.11 mmol) was added. The reaction mixture was stirred at 0° C. for 16 h. The progress of reaction, was monitored by TLC. After completion, reaction mixture was quenched with triethyl amine. To the reaction mixture, cold water (200 mL) was added and extracted with diethyl ether. The combined organic layer dried over anhydrous sodium sulphate, filtered and concentrated under reduced pressure to get crude (2) as white solid. The crude was used for the next step. Yield: 43.0 g, Crude; LCMS m/z. 261.15 [M+1]⁺.

To a solution of (2, 43.0 g, 1.0 eq, 165.0 mmol) in dry toluene (1000 mL), triphenyl phosphine (52.0 g, 1.2 eq, 198.0 mmol) and 4-nitrophenol (27.6 g, 1.2 eq, 198.0 mmol) were added and reaction mixture stirred for 5 minutes, then at 0° C., diisopropyl azodicarboxylate (41.0 mL, 1.2 eq, 198.0 mmol) was added and reaction mixture was stirred at room temperature for 1 h. The progress of reaction was monitored by TLC. After completion, the reaction mixture was quenched with 10% aq. sodium hydroxide, extracted with ethyl acetate. The combined organic layer dried over anhydrous sodium sulphate, filtered and concentrated under reduced pressure to get crude. The crude was purified by silica gel flash column chromatography using 2-10% ethylacetate in hexane as eluent to afford (3) as white solid. Yield: 16.5 g (26%) LCMS m/z. 382.20 [M+1]⁺.

To a stirred solution of (3, 15.0 g, 1.0 eq, 39.3 mmol) in methanol (150 mL), Dowex-50 H⁺ (20.0 g) was added and reaction mixture was stirred at room temperature for 6 h. After completion, reaction mixture was filtered through sintered funnel. The filtrate was concentrated under reduced pressure to get (4) as white semi solid. Yield: 9.6 g, Crude; LCMS m/z. 382.20 [M+Na]⁺.

To a stirred solution of (4, 5.00 g, 16.6 mmol) in N,N-dimethylformamide (30 mL) was cooled to 0° C. Then triethylamine (27.0 mL, 12.0 eq., 199 mmol) and chlorotrimethylsilane (12.6 mL, 6.0 eq, 99.6 mmol) were added under nitrogen atmosphere. The resulting mixture was stirred at room temperature under nitrogen for 24 h. The reaction mixture was partitioned between ethyl acetate and water. The water layer was extracted again with ethyl acetate. The combined organic layers were dried over sodium sulfate, filtered, and concentrated under reduced pressure to afford crude. Which was purified by silica gel flash column chromatography using 20-40% ethyl acetate in hexane as eluent to afford (5) as colorless gel. Yield: 8.0 g, 79.0%; LCMS m/z. 607.15 [M+18]⁺.

To a stirred solution of (5, 5.5 g, 9.32 mmol) in methanol (30 mL) and dichloromethane (30 mL), ammonium acetate (1.08 g, 1.50 eq, 14.0 mmol) was added at room temperature under nitrogen. The resulting mixture was stirred at room temperature under nitrogen for 16 h. After completion, reaction mixture was, concentrated under reduced pressure to get crude. The crude was purified by silica gel flash column chromatography using 20-40% ethyl acetate in hexane as eluent to afford (6) as white solid. Yield: 3.50 g, 72% LCMS m/z. 535.0 [M+18]⁺.

To a stirred solution of oxalic dichloride (0.642 mL, 1.1 eq, 7.44 mmol) in dichloromethane (25 mL) at −78° C. was added a solution of dimethylsulfoxide (1.06 mL, 2.2 eq, 14.9 mmol) in dichloromethane (5 mL) over 5 minutes. After being stirred at −78° C. for 20 minutes, a solution of (6, 3.5 g, 6.76 mmol) in dichloromethane (10 mL) was added to the mixture. The reaction mixture was further stirred at −78° C. for 1 h, followed by addition of triethylamine (4.75 mL, 5.0 eq, 33.8 mmol). The resulting mixture was allowed to reach room temperature over 1 h. The reaction mixture was diluted with dichloromethane and washed with water followed by brine solution. The organic layer was dried over anhydrous sodium sulfate, filtered, and concentrated under reduced pressure to afford (7) as brown syrup. Yield: 3.50 g Crude; LCMS m/z. 533.0 [M+18]⁺.

To a stirred solution of diethyl [(diethoxyphosphoryl)methyl]phosphonate (7a, 2.93 mL, 1.5 eq, 10.2 mmol) in tetrahydrofuran (40.0 mL) in −78° C., n-butyllithium (2.5 M in hexane) (5.09 mL, 1.5, 10.2 mmol) was added drop wise under nitrogen atmosphere and stirred at −78° C. for 1 h. Then, (7, 3.55 g, 1.0 eq, 6.79 mmol) dissolved in tetrahydrofuran (10.0 mL) was added drop wise and reaction mixture was allowed to warm up to room temperature and stirred for 16 h. After completion of reaction, reaction mixture was quenched with saturated ammonium chloride solution and extracted with ethyl acetate. The combined organic layer was washed with brine and dried over sodium sulfate, filtered and concentrated to under reduced pressure get crude. The crude was further purified by silica gel flash column chromatography using 15-60% ethyl acetate in hexane as eluent to afford (8) as a colorless liquid. Yield: 1.85 g, 42%; LCMS m/z. 650.27 [M+1]⁺.

To a stirred solution of (8, 1.80 g, 2.77 mmol)) in methanol (20 mL). was added Dowex 50WX8 (1.80 g) at room temperature under nitrogen atmosphere. The resulting mixture was stirred at room temperature under nitrogen for 2 h. The reaction mixture filtered and washed with methanol, filtrate concentrated under vacuum to (9) as a colorless liquid. Yield: 1.18 g, 98%; LCMS m/z 433.95 [M+1]⁺.

To a stirred solution of (9, 1.18 g, 1.0 eq, 2.72 mmol) in pyridine (10.0 mL), cooled to 0° C. and then acetic anhydride (4.09 mL, 15.0 eq, 40.8 mmol) was added drop wise and reaction mass was stirred at room temperature for 16 h. After completion of reaction, reaction mixture was evaporated under reduced vacuum and then diluted with ethyl acetate. Organic layer was washed with water followed by saturated aqueous sodium bicarbonate solution. The combined organic layer was dried over sodium sulfate, filtered and concentrated to get crude liquid. It was further purified by column chromatography (eluted in 20-40% ethyl acetate in hexane) to afford (10) as a colorless liquid. Yield: 1.3 g, 84%; LCMS m/z 560.10 [M+1]⁺.

To a stirred solution of (10, 1.30 g, 2.32 mmol) in dichloromethane (20.0 mL), 10% palladium on carbon (1.40 g) was added and then reaction mixture was stirred under hydrogen gas atmosphere (balloon pressure) for 16 h. Reaction monitored by TLC and LC-MS. After completion of reaction, reaction mixture was filtered through syringe filters. The combined filtrate was evaporated under reduced pressure to afford (11) as a brown gel. Yield: 1.10 g (Crude) LCMS m/z 531.95 [M+1]⁺.

To a solution of (11, 0.40 g, 1.0 eq 0.753 mmol) in N,N-dimethyl formamide (5.0 mL), N,N-diisopropylethyl amine (1.31 mL, 10.0 eq, 7.53 mmol) and 4-nitrophenyl hex-5-yn-1-ylcarbamate (11a, 3.0 eq, 0.592 g, 2.26 mmol) were added. The reaction mixture was stirred at room temperature for 16 h. The progress of reaction was monitored by LCMS. The reaction mixture was concentrated under reduced pressure to afford crude. The crude was purified by reverse phase (Aq C-18 column) column chromatography using 20-50% acetonitrile in water as eluent. The fractions were washed with ethyl acetate. The organic layer dried over anhydrous sodium sulphate, filtered and concentrated under reduced pressure to afford (12) as brown sticky solid. Yield: 0.38 g, 39%; LCMS m/z. 654.95 [M+1]⁺.

To a stirred solution of (12, 0.37 g, 1.0 eq, 0.339 mmol) in dichloromethane (10.0 mL) and pyridine (0.27 mL, 10.0 eq, 3.39 mmol), cooled to 0° C. and bromotrimethylsilane (0.44 mL, 10.0 eq., 3.39 mmol) was added and reaction mixture was stirred at room temperature for 16 h. After completion, reaction mixture was concentrated under reduced vacuum to get off-white solid. It was further washed with di-ethyl ether and dried to afford (13) as an off white solid. Yield: 0.25 g, Crude; LCMS m/z 599.15 [M+1]⁺.

To a stirred solution of (2-((2R,3R,4S,5S,6S)-3,4,5-triacetoxy-6-(4-(3-(hex-5-yn-1-yl)ureido)phenoxy)tetrahydro-2H-pyran-2-yl)ethyl)phosphonic acid (13, 0.25 g, 1.0 eq, 0.418 mmol) in methanol (5.0 mL), sodium methoxide (25% in methanol, 0.63 mL, 7.0 eq, 2.92 mmol) was added and reaction mixture was stirred at room temperature for 2 h. Reaction was monitored by LC-MS. After completion of reaction, reaction mixture was concentrated under reduced pressure to get crude. The crude was purified by reverse phase column chromatography (using 20-32% ACN in water with 0.1% TFA) to afford (2-((2R,3S,4S,5S,6S)-6-(4-(3-(hex-5-yn-1-yl)ureido)phenoxy)-3,4,5-trihydroxytetrahydro-2H-pyran-2-yl)ethyl)phosphonic acid (566A) as a white solid. Yield: 0.025 g (13%) LCMS m/z 473.20 [M+1]; ¹H NMR (400 MHz, DMSO with D₂O exchange) δ 7.24 (d, J=8.88 Hz, 2H), 6.83 (d, J=9.2 Hz, 2H), 4.98 (s, 1H),), 3.81 (d, J=3.2 Hz, 1H), 3.36-3.33 (dd, J=3.2, & 9.2, Hz, 1H), 3.22 (t, J=9.2 Hz, 1H), 3.15 (t, J=7.6 Hz, 1H), 3.06 (t, J=6.0 Hz, 2H) 2.69-2.66 (m, 1H), 2.17-2.14 (m, 2H), 2.02-1.99 (m, 1H), 1.65-1.61 (m, 1H), 1.50-1.42 (m, 6H).

### Synthesis of Synthon 642A

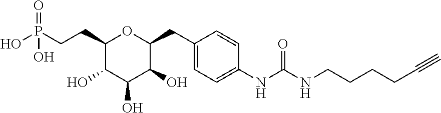 [SMILES: C#CCCCCNC(=O)Nc1ccc(C[C@@H]2O[C@H](CCP(=O)(O)O)[C@@H](O)[C@H](O)[C@@H]2O)cc1]

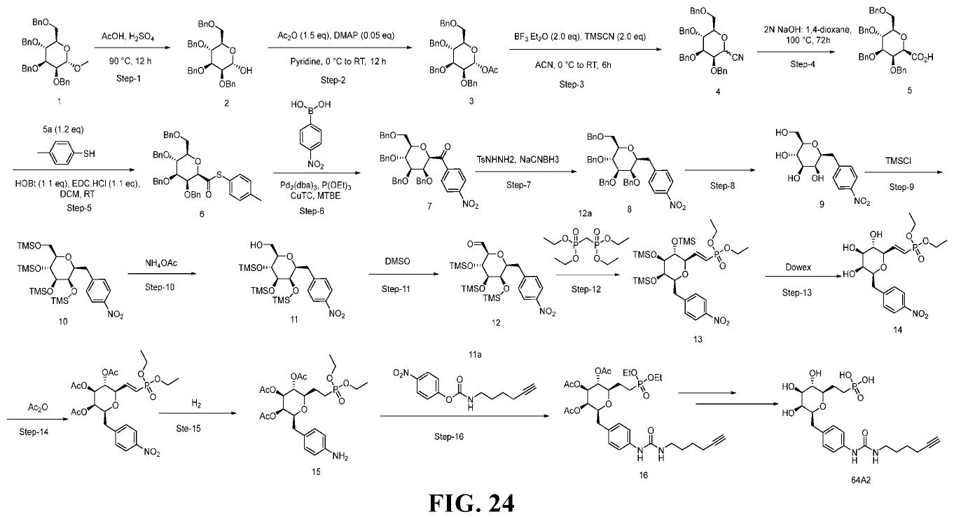

(2-((2R,3S,4R,5S,6S)-6-(4-(3-(hex-5-yn-1-yl)ureido)benzyl)-3,4,5-trihydroxytetrahydro-2H-pyran-2-yl)ethyl)phosphonic acid (642). See synthetic scheme of FIG. 24. The synthesis of the title compound can be accomplished through the above scheme where (2R,3R,4S,5S,6S)-3,4,5-tris(benzyloxy)-2-((benzyloxy)methyl)-6-methoxytetrahydro-2H-pyran (1) is first converted to (2R,3S,4R,5S,6S)-2-(hydroxymethyl)-6-(4-nitrobenzyl)tetrahydro-2H-pyran-3,4,5-triol (9). Compound 9 can be converted to 642A using chemistry exemplified in the examples Compound A and 1-38.

### Synthesis of Synthon 643A

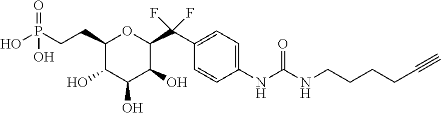 [SMILES: C#CCCCCNC(=O)Nc1ccc(C(F)(F)[C@@H]2O[C@H](CCP(=O)(O)O)[C@@H](O)[C@H](O)[C@@H]2O)cc1]

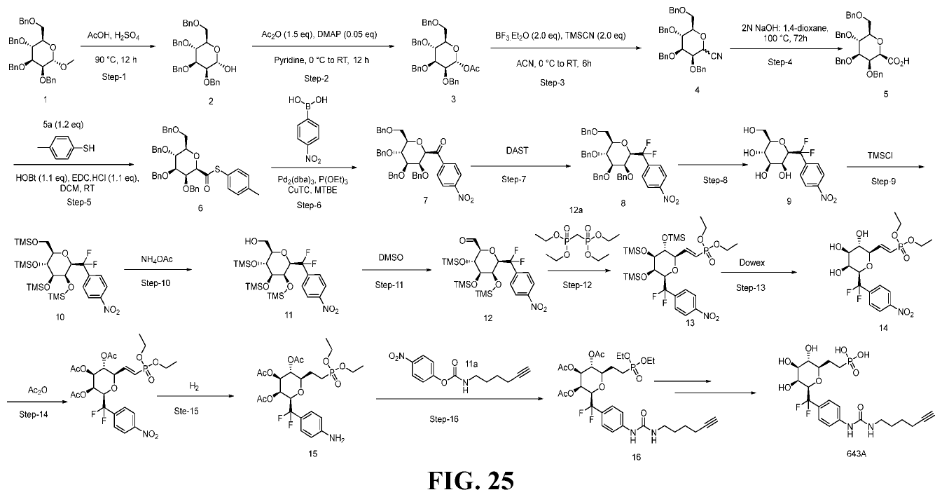

(2-((2R,3S,4S,5S,6R)-6-(difluoro(4-(3-(hex-5-yn-1-yl)ureido)phenyl)methyl)-3,4,5-trihydroxytetrahydro-2H-pyran-2-yl)ethyl)phosphonic acid (643A). See synthetic scheme of FIG. 25. The synthesis of the title compound can be accomplished through the above scheme where (2R,3R,4S,5S,6S)-3,4,5-tris(benzyloxy)-2-((benzyloxy)methyl)-6-methoxytetrahydro-2H-pyran (1) is first converted to (2R,3S,4S,5S,6R)-2-(difluoro(4-nitrophenyl)methyl)-6-(hydroxymethyl)tetrahydro-2H-pyran-3,4,5-triol (9). Compound 9 can be converted to 643A using chemistry exemplified in the examples Compound A and 1-38.

Synthesis of M6PR ligand Probes

The compounds in Table 13 were prepared from starting material indicated and 3-azido pyridine by adapting the methods described herein.

TABLE 13
exemplary M6PR ligand probes P1-P5

| | | | LCMS |
|---|---|---|---|
| Cpd# | Structure | SM used | m/z |
| P1 | 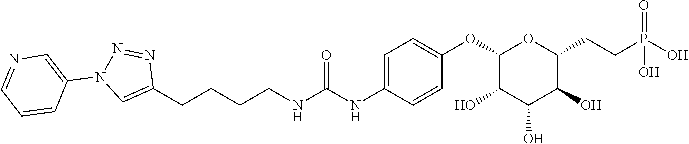 [SMILES: O=C(NCCCCc1cn(-c2cccnc2)nn1)Nc1ccc(O[C@@H]2O[C@H](CCP(=O)(O)O)[C@@H](O)[C@H](O)[C@@H]2O)cc1] | 566A | 593.1 [M + H]⁺ |
| P2 | 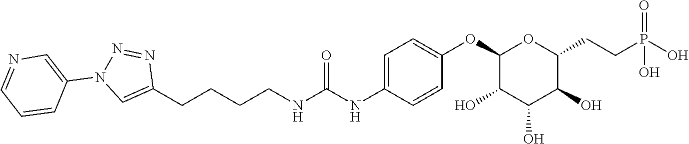 [SMILES: O=C(NCCCCc1cn(-c2cccnc2)nn1)Nc1ccc(O[C@H]2O[C@H](CCP(=O)(O)O)[C@@H](O)[C@H](O)[C@@H]2O)cc1] | 38C | 593.1 [M + H]⁺ |
| P3 | 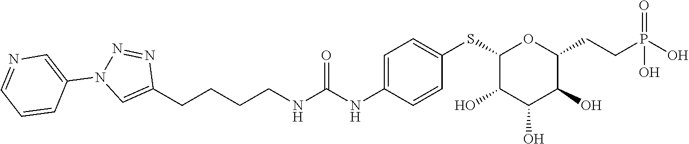 [SMILES: O=C(NCCCCc1cn(-c2cccnc2)nn1)Nc1ccc(S[C@@H]2O[C@H](CCP(=O)(O)O)[C@@H](O)[C@H](O)[C@@H]2O)cc1] | I-102 | 609.1 [M + H]⁺ |
| P4 | 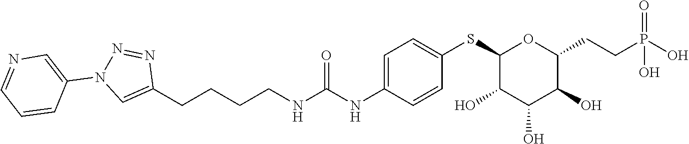 [SMILES: O=C(NCCCCc1cn(-c2cccnc2)nn1)Nc1ccc(S[C@H]2O[C@H](CCP(=O)(O)O)[C@@H](O)[C@H](O)[C@@H]2O)cc1] | I-67 | 609.1 [M + H]⁺ |
| P5 | 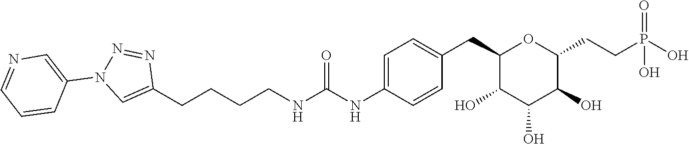 [SMILES: O=C(NCCCCc1cn(-c2cccnc2)nn1)Nc1ccc(C[C@H]2O[C@H](CCP(=O)(O)O)[C@@H](O)[C@H](O)[C@@H]2O)cc1] | I-101 | 591.1 [M + H]⁺ |

### 5.1.2. Preparation of M6PR Ligand-Linker Compounds

### Example 1: Synthesis of Compound I-1 from Amino Intermediate A-10

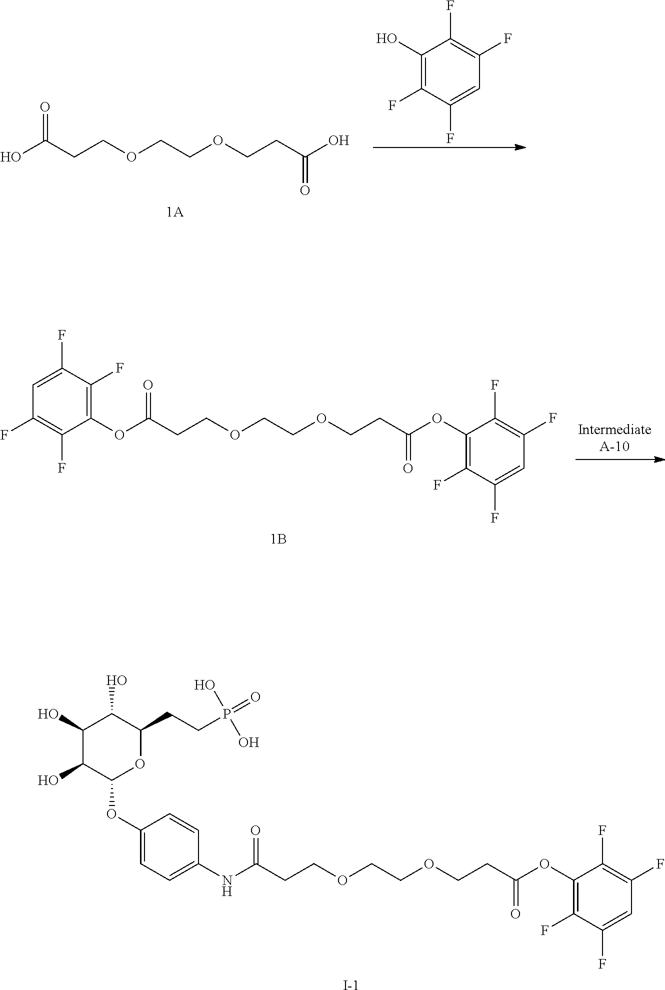 [SMILES: O=C(O)CCOCCOCCC(=O)O ; Oc1c(F)c(F)cc(F)c1F ; O=C(CCOCCOCCC(=O)Oc1c(F)c(F)cc(F)c1F)Oc1c(F)c(F)cc(F)c1F ; O=C(CCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1 ; [I-]]

A solution of 3,3′-(ethane-1,2-diylbis(oxy))dipropionic acid (1A) (1.0 eq, 0.200 g, 0.96 mmol) and 2,3,5,6-tetrafluorophenol (2.0 eq, 0.315 g, 1.9 mmol) in ethyl acetate (4 mL) was cooled at 0° C., N,N′-diisopropylcarbodiimide (3.0 eq, 0.44 mL, 2.8 mmol) was added and reaction mixture was stirred at room temperature for 3 h. Reaction mixture was filtered through Celite bed and Celite bed was washed with ethyl acetate. The filtrate was concentrated to get crude product which was purified by column chromatography using silica gel (100-200 mesh) and 0-10% ethyl acetate in hexane to afford Compound 1B as a colorless viscous liquid. Yield: 0.370 g, 76.1%; LC-MS m/z 500.96 [M−1]⁻.

Intermediate A-10 (1.0 eq, 0.040 g, 0.11 mmol) was dissolved in dimethyl sulfoxide (1 mL) and triethylamine (10.0 eq, 0.15 mL, 1.1 mmol) was added. In another vial, Compound 1B (5.0 eq, 0.276 g, 0.55 mmol) was dissolved in dimethyl sulfoxide (1 mL) and the previous mixture was added dropwise to this mixture (over 30 minutes). Reaction mixture was stirred at room temperature for 5 minutes. After completion, reaction mixture was diluted with acetonitrile and purified by preparatory HPLC (25-45% acetonitrile in water with 0.1% TFA). Fractions containing the desired product were combined and lyophilized to dryness to afford Compound I-1 as an off white solid. Yield: 0.002 g, 2.5%; LC-MS m/z 686.25 [M+1]+; ¹H NMR (400 MHz, D₂O) δ 7.35 (d, J=8.88 Hz, 2H), 7.29-7.23 (m, 1H), 7.07 (d, J=8.96 Hz, 2H), 5.50 (s, 1H), 4.13 (bs, 1H), 3.98-3.95 (m, 1H), 3.91 (t, J=5.64 Hz, 2H), 3.86 (t, J=5.72 Hz, 2H), 3.72 (s, 4H), 3.58 (d, J=7.32 Hz, 2H), 2.96 (t, J=5.76 Hz, 2H), 2.66 (t, J=5.8 Hz, 2H), 2.03-2.00 (m, 1H), 1.74-1.63 (m, 2H), 1.32-1.26 (m, 1H).

### Example 2: Synthesis of Compound I-2

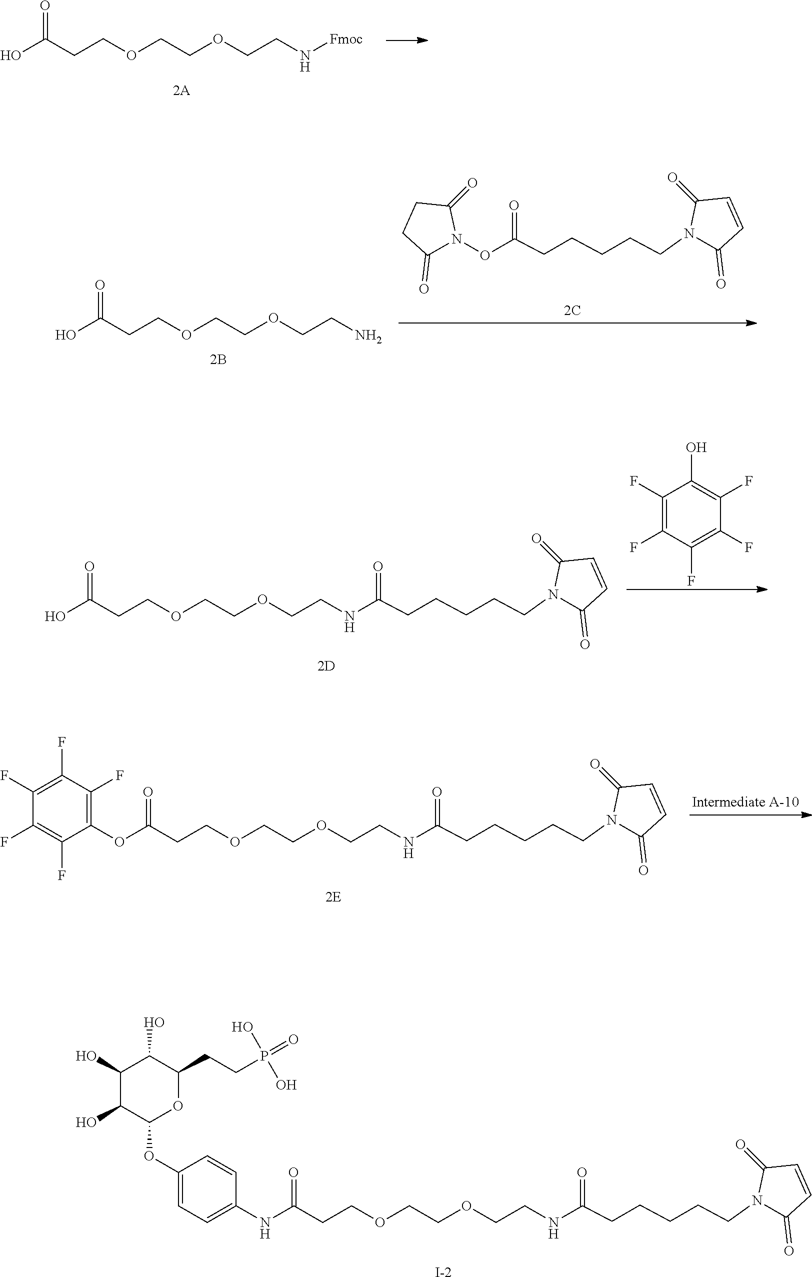

Compound I-2 was prepared using similar methods. Yield: 0.03 g, 31%; LC-MS m/z 702.31 [M+1]. ¹H NMR (400 MHz, D₂O) δ 7.37 (d, J=8.8 Hz, 2H), 7.11 (d, J=8.9 Hz, 2H), 6.77 (s, 2H), 5.52 (d, J=1.48 Hz, 1H), 5.20 (bs, 1H), 4.14-4.13 (m, 1H), 3.98-3.95 (m, 1H), 3.85 (t, J=5.88 Hz, 2H), 3.72-3.66 (m, 6H), 3.60-3.55 (m, 4H), 3.42 (t, J=6.96 Hz, 2H), 3.29 (t, J=5.28 Hz, 2H), 2.66 (t, J=5.84 Hz, 2H), 2.10 (t, J=7.32 Hz, 2H), 2.04-1.95 (m, 1H), 1.69-1.57 (in 2H), 1.54-1.45 (m, 4H).

### Example 3: Synthesis of Compound I-3

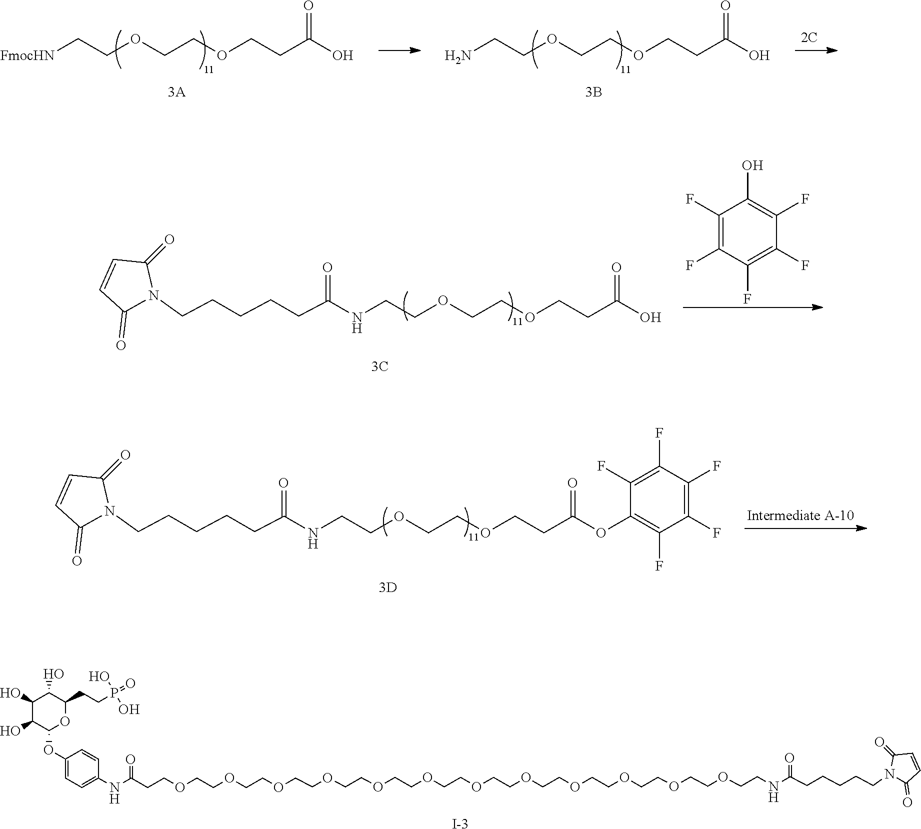

Compound I-3 was prepared using similar methods (0.011 g, 10% yield). LC-MS m/z 1142.6 [M+1]. H NMR (400 MHz, D₂O) δ 7.27 (d, J=9.0 Hz, 2H), 7.16 (d, J=9.0 Hz, 2H) 6.83 (s, 2H), 5.56 (s, 1H), 4.20-4.15 (m, 1H), 4.05-3.98 (m, 1H), 3.88 (t, J=6.0 Hz, 2H), 3.75-3.58 (m, 49H), 3.49 (t, J=6.8 Hz, 2H), 3.37 (t, J=5.6 Hz, 2H), 2.69 (t, J=6.0 Hz, 2H), 2.23 (t, J=7.2 Hz, 2H), 2.10-1.98 (m, 1H), 1.75-1.55 (m, 6H), 5.56 (s, 1H), 1.38-1.20 (m, 2H).

### Example 4: Synthesis of Compound I-4

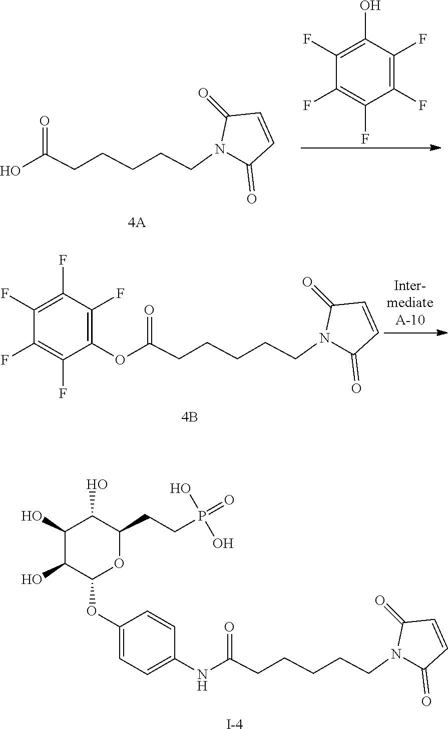

Compound I-4 was prepared using similar methods. Yield: 0.006 g, 8.0%; LC-MS m/z 543.27 [M+1]+; ¹H NMR (400 MHz, D₂O) δ 7.35 (d, J=8.96 Hz, 2H), 7.16 (d, J=9.0 Hz, 2H), 6.77 (s, 2H), 5.58 (d, J=1.64 Hz, 1H), 4.17-4.16 (m, 1H), 4.02-3.95 (m, 1H), 3.63-3.57 (m, 2H), 3.52 (t, J=6.84 Hz, 2H), 2.39 (t, J=7.24 Hz, 2H), 2.06-1.98 (m, 1H), 1.74-1.58 (m, 6H), 1.37-1.22 (m, 3H).

### Example 5: Synthesis of Compound I-5

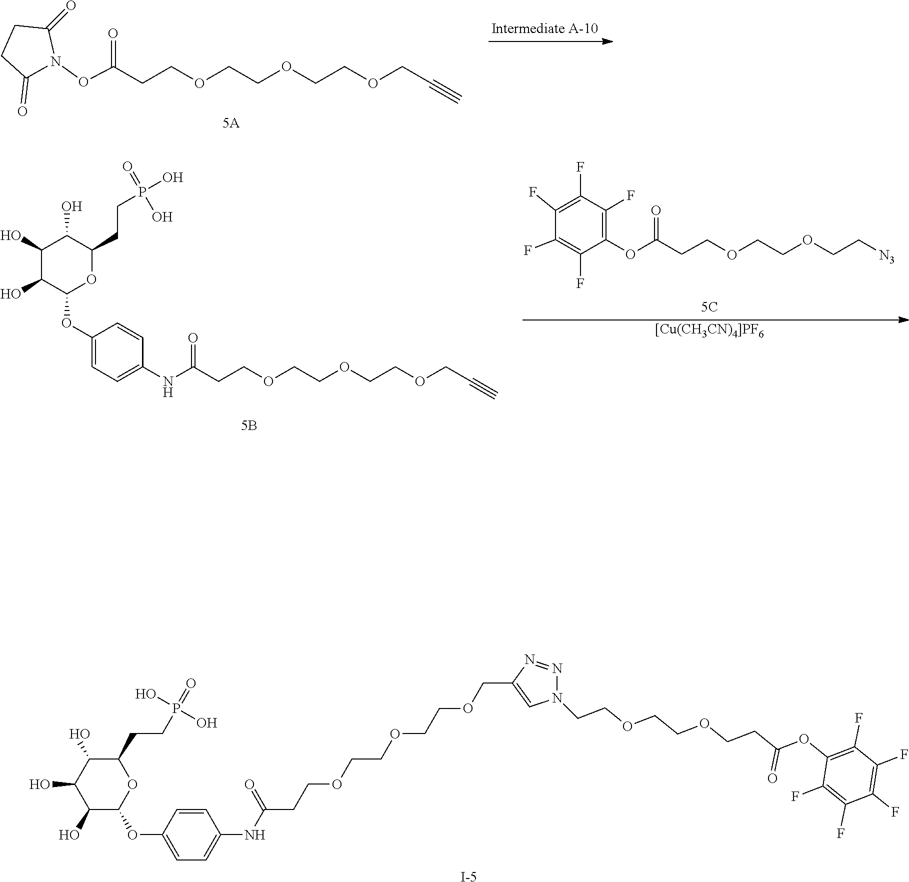

In dimethylsulfoxide (1.0 mL), molecular sieves (Powder, Catalyst support, sodium Y zeolite, Aldrich Cat no. 334448) was added followed by Intermediate A-10 (1.0 eq, 0.060 g, 0.172 mmol), triethylamine (3.0 eq, 0.074 mL, 0.515 mmol) and 2,5-dioxopyrrolidin-1-yl 3-(2-(2-(prop-2-yn-1-yloxy)ethoxy)ethoxy)propanoate (5A) (1.0 eq, 0.053 g, 0.172 mmol) were added and reaction mixture was stirred at room temperature for 3 h. After completion, reaction mixture was diluted with acetonitrile and purified by preparatory HPLC (14-33% acetonitrile in water with 0.1% TFA). Fractions containing the desired product were combined and lyophilized to dryness to afford Compound 5B as an off white sticky solid. Yield: 0.018 g, 17.93%; LC-MS m/z 548.32 [M+1]⁺.

A solution of Compound 5B (1.0 eq, 0.018 g, 0.032 mmol) and perfluorophenyl 3-(2-(2-azidoethoxy)ethoxy)propanoate (5C) (1.2 eq, 0.014 g, 0.039 mmol) in dimethyl sulfoxide (0.6 mL) was stirred at room temperature for 5 minutes. Then, tetrakis(acetonitrile)copper(I) hexafluorophosphate (2.8 eq, 0.034 g, 0.092 mmol) was added and reaction mixture was stirred at room temperature for 1 h. After completion, reaction mixture was diluted with acetonitrile and purified by prep-HPLC (40-60% acetonitrile in water with 0.1% TFA). Fractions containing the desired product were combined and lyophilized to dryness to afford Compound I-5 as a white solid. Yield: 0.015 g, 48.67%; LC-MS m/z 917.37 [M+1]+; ¹H NMR (400 MHz, D₂O) δ 7.97 (s, 1H), 7.36 (d, J=9.2 Hz, 2H), 7.08 (d, J=9.2 Hz, 2H), 5.51 (s, 1H), 4.59-4.55 (m, 2H), 4.15-4.14 (m, 1H), 3.97-3.92 (m, 3H), 3.87-3.81 (m, 4H), 3.70-3.57 (m, 14H), 2.97 (t, J=6.0 Hz, 2H), 2.66 (t, J=6.0 Hz, 2H), 2.00 (bs, 1H), 1.71-1.64 (m, 2H), 1.33 (bs, 1H).

### Example 6: Synthesis of Compound I-6

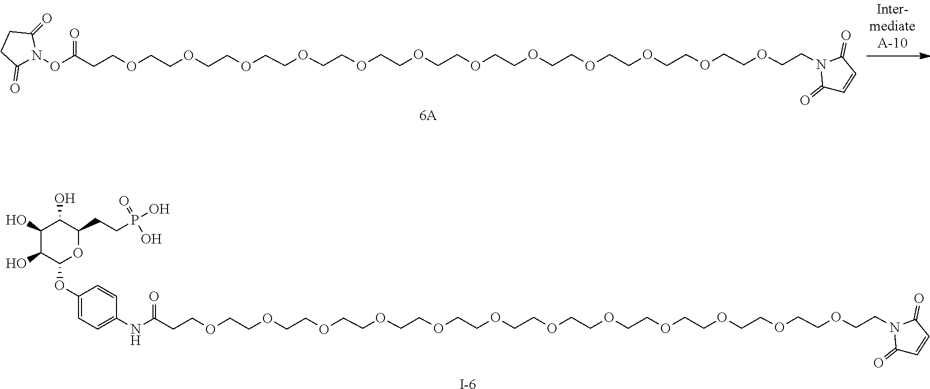

Compound I-6 was prepared using similar methods. Yield: 0.0065 g, 11%; LC-MS m/z 1029.58 [M+1]⁺. ¹H NMR (400 MHz, D₂O) δ 7.42 (d, J=8.8 Hz, 2H), 7.16 (d, J=9.2 Hz, 2H), 6.86 (s, 2H), 5.56 (s, 1H), 4.16 (d, J=1.6 Hz 1H), 4.00 (t, J=9.6 Hz 1H), 3.87 (t, J=5.88 Hz, 2H), 3.71-3.61 (m, 50H), 2.69 (t, J=11.6 Hz, 2H), 2.15-1.95 (m, 1H), 1.75-1.61 (m, 2H), 1.43-1.25 (m, 1H).

### Example 7: Synthesis of Compound I-7

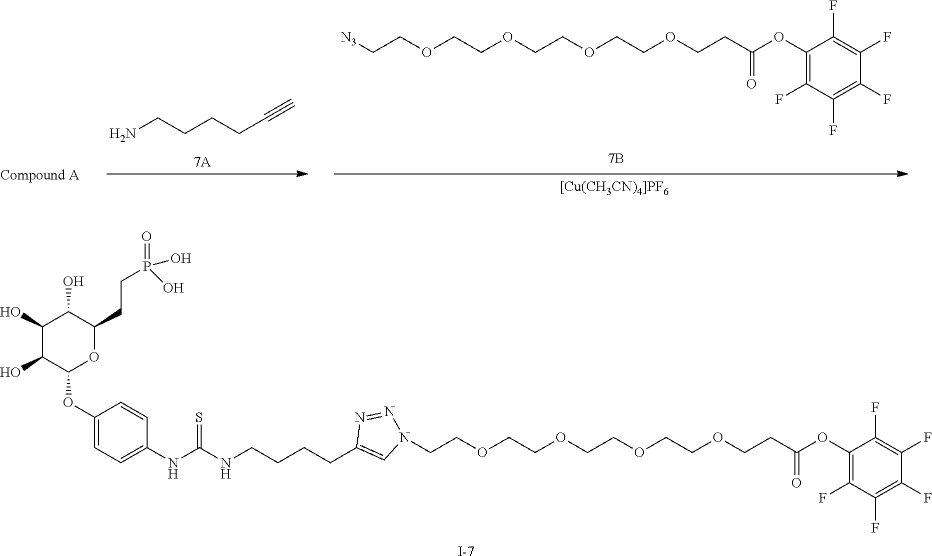

Compound I-7 was prepared using similar methods. Yield: 11.1 mg, 35%; LC-MS m/z 946.5 [M+1]+; ¹H NMR (300 MHz, DMSO-d₆ with D₂O) δ 7.80 (s, 1H), 7.25 (d, J=8.4 Hz, 2H), 6.98 (d, J=8.4 Hz, 2H), 5.32 (s, 1H), 4.44 (s, 2H), 3.86-3.68 (m, 5H), 3.67-3.23 (m, 17H), 3.05-2.91 (m, 2H), 2.67-2.56 (m, 2H), 2.00-1.81 (m, 1H), 1.69-1.41 (m, 6H), 1.30-1.07 (m, 1H).

### Example 8: Synthesis of Compound I-8

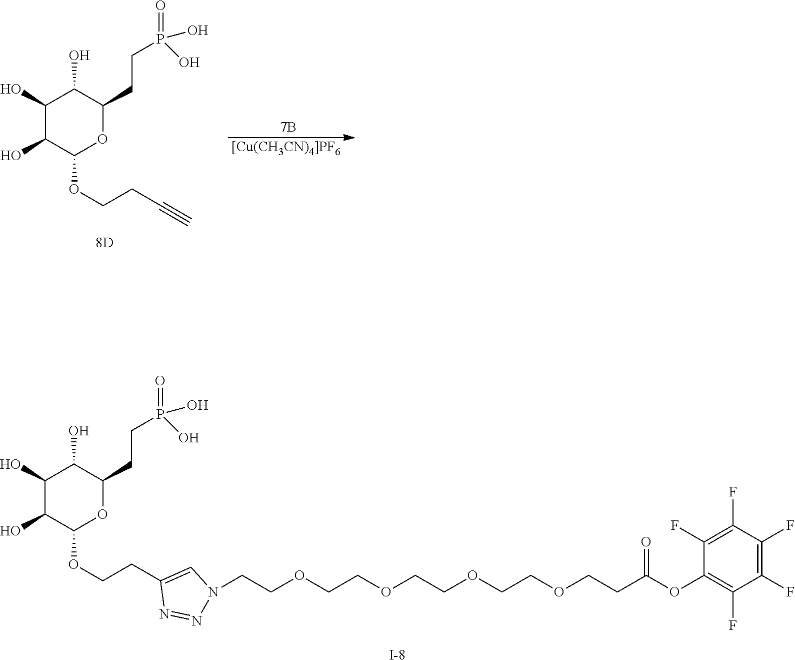

Compound 8D (1.00 eq, 10.0 mg, 0.032 mmol) and azido-PEG4-pentafluorophenol ester 7B (1.20 eq, 17.7 mg, 0.039 mmol) were dissolved in NMP (0.3 mL) with stirring. After 2 min tetrakis(acetonitrile)copper(I) hexafluorophosphate (2.80 eq, 33.6 mg, 0.090 mmol) was added. The resulting light yellow solution was capped and stirred at room temperature for 30 min (slowly turned more green-colored). The reaction mixture was diluted with mixture of NMP, ethanol, and acetic acid, filtered, and purified via preparatory HPLC (15-65% acetonitrile in water with 0.1% TFA). Fractions containing the desired product were combined and lyophilized to dryness to afford Compound I-8 as a white solid. Yield: 12.3 mg, 50%; LC-MS m/z 768.5 [M+1]+; H NMR (300 MHz, DMSO-d₆) δ 7.81 (s, 1H), 4.59 (s, 1H), 4.44 (bs, 2H), 3.60-3.30 (m, 17H), 3.27-2.76 (m, 9H), 2.01-1.84 (m, 1H), 1.77-1.58 (m, 1H), 1.56-1.32 (m, 2H).

### Example 9: Synthesis of Compound I-9

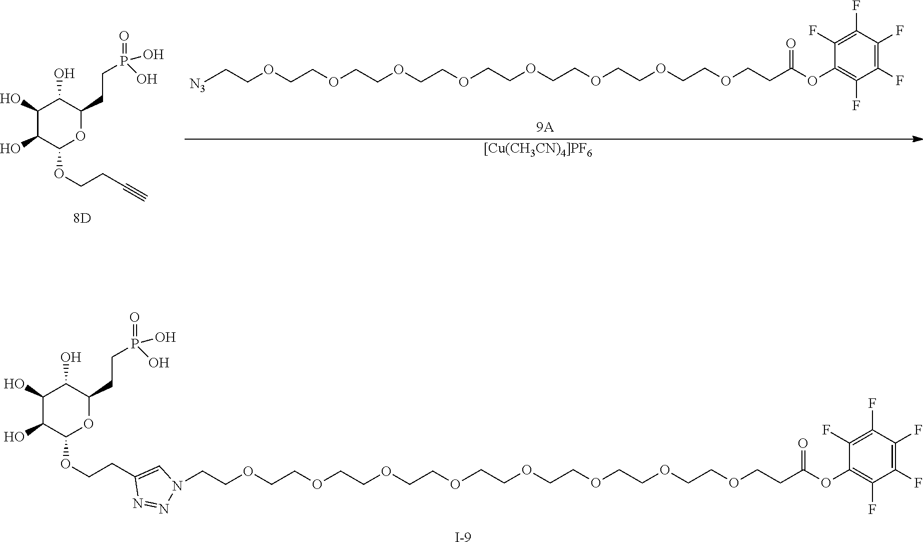

Compound I-9 was prepared using similar methods. Yield: 18.9 mg, 63%; LC-MS m/z 944.6 [M+1]+; ¹H NMR (300 MHz, DMSO-d₆ with D₂O) δ 7.81 (s, 1H), 4.59 (s, 1H), 4.44 (s, 2H), 3.86-3.29 (m, 34H), 3.29-2.69 (m, 8H), 2.01-1.80 (m, 1H), 1.80-1.57 (m, 1H), 1.56-1.30 (m, 2H).

### Example 10: Synthesis of Compound I-10

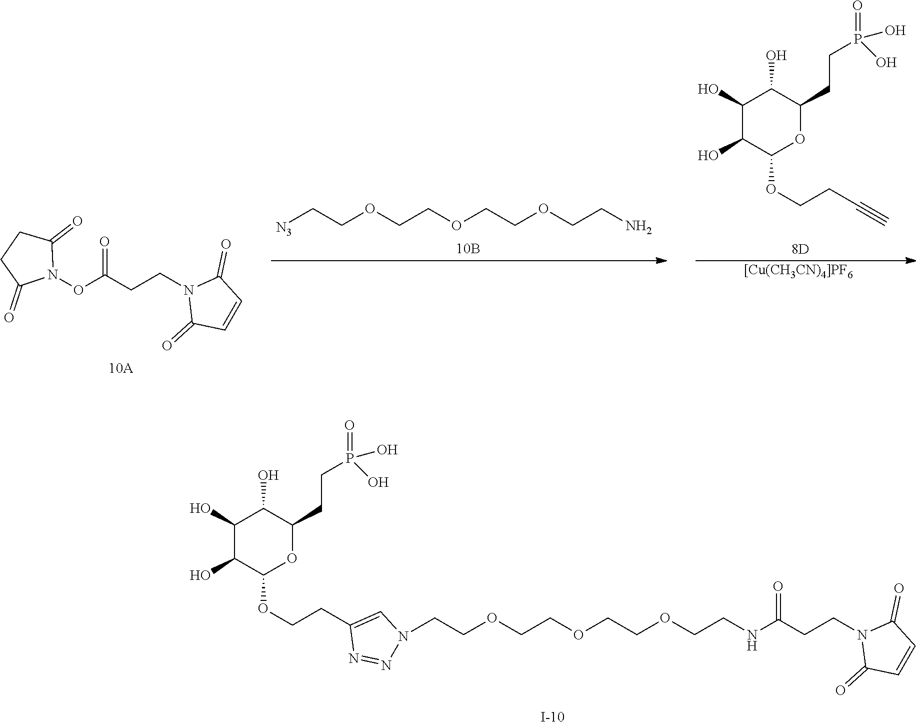

Compound I-10 was prepared using similar methods. Yield: 17.7 mg, 52%; LC-MS m/z 680.5 [M+1]+; ¹H NMR (300 MHz, DMSO-d₆ with D₂O) δ 7.81 (s, 1H), 6.92 (s, 2H), 4.59 (s, 1H), 4.44 (s, 2H), 3.63-3.26 (m, 15H), 3.26-2.70 (m, 9H), 2.36-2.21 (m, 2H), 2.05-1.83 (m, 1H), 1.79-1.60 (m, 1H), 1.54-1.30 (m, 2H).

### Example 11: Synthesis of Compound I-11

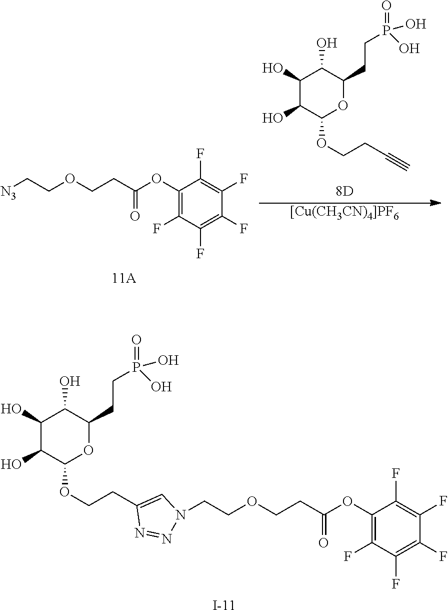

Compound I-11 was prepared using similar methods. Yield: 14.9 mg, 54%; LC-MS m/z 636.4 [M+1]+; ¹H NMR (300 MHz, DMSO-d₆ with D₂O) δ 7.75 (s, 1H), 4.57 (s, 1H), 4.51-4.35 (m, 2H), 3.84-3.65 (m, 5H), 3.60-3.45 (m, 2H), 3.41-3.29 (m, 1H), 3.21 (t, J=9.3 Hz, 1H), 3.15-3.03 (m, 1H), 3.03-2.88 (m, 2H), 2.88-2.74 (m, 2H), 2.02-1.82 (m, 1H), 1.79-1.59 (m, 1H), 1.56-1.28 (m, 2H).

### Example 12: Synthesis of Compound I-12

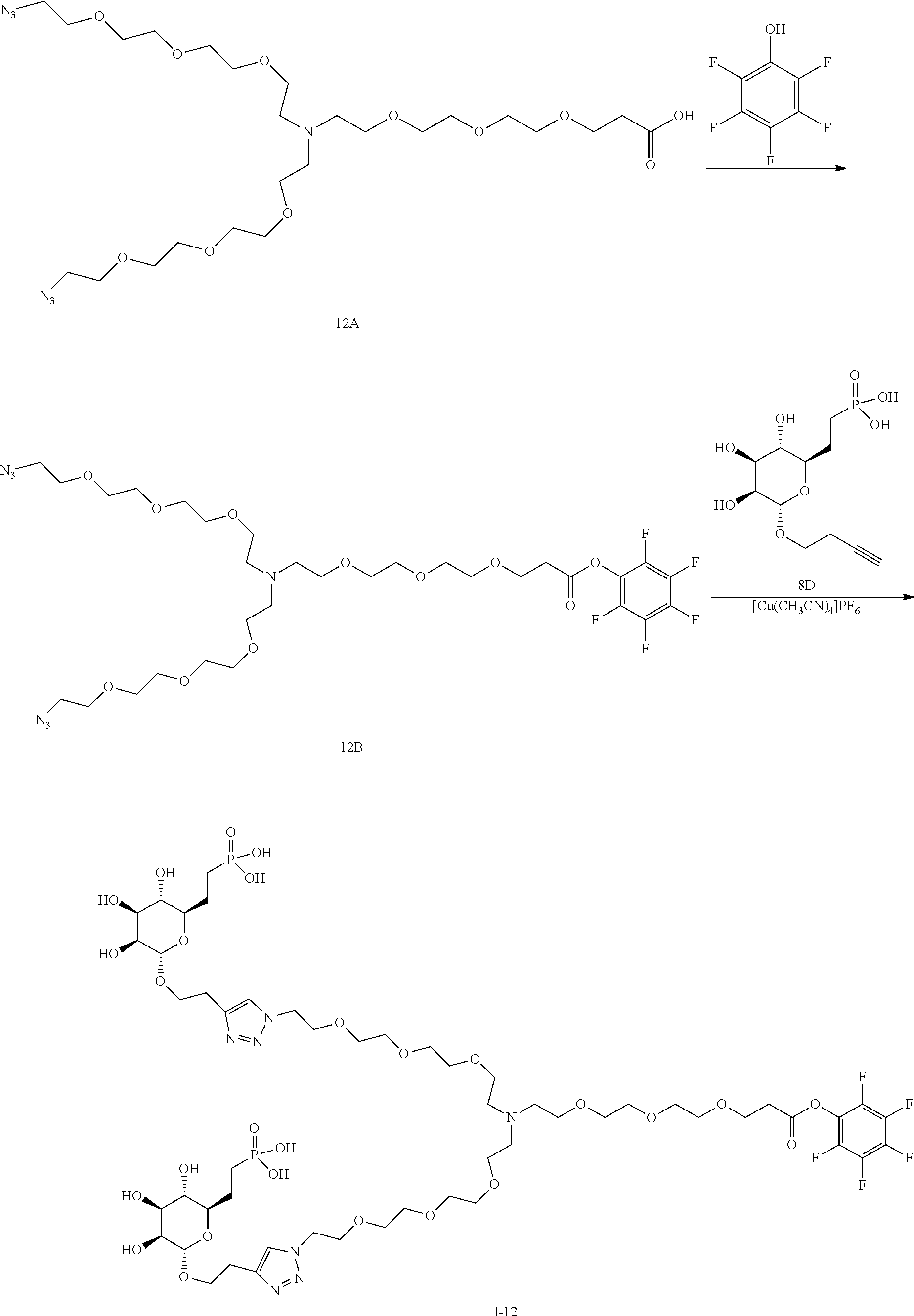

Compound I-12 was prepared using similar methods. Yield: 8.7 mg, 21%; LC-MS m/z 1410.9 [M+1]+; ¹H NMR (300 MHz, DMSO-d₆ with D₂O) δ 7.81 (s, 2H), 4.60 (s, 2H), 4.45 (s, 4H), 3.87-2.76 (m, 50H), 2.03-1.83 (m, 2H), 1.79-1.59 (m, 2H), 1.55-1.29 (m, 4H).

### Example 13: Synthesis of Compound I-13

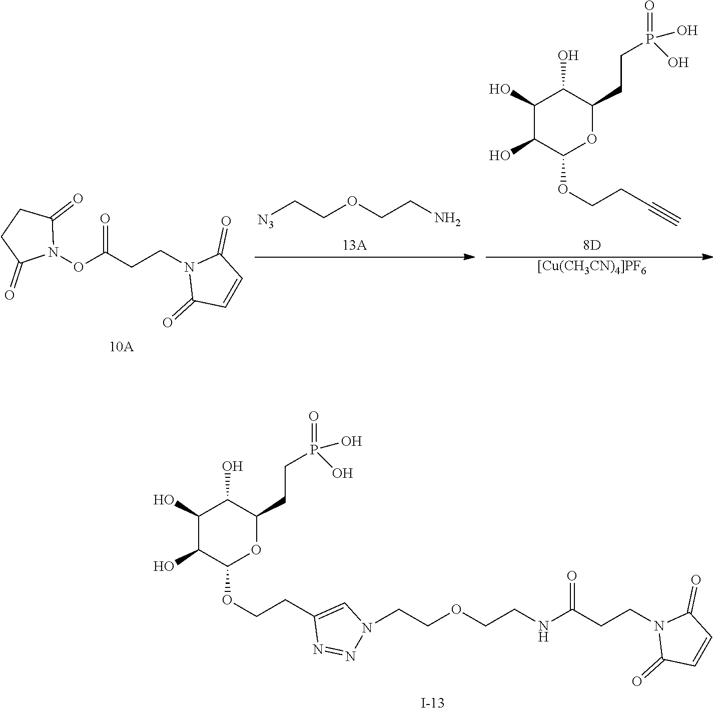

Compound I-13 was prepared using similar methods. Yield: 15.0 mg, 51%; LC-MS m/z 592.4 [M+1]+; ¹H NMR (300 MHz, DMSO-d₆ with D₂O) δ 7.81 (s, 1H), 6.95 (s, 2H), 4.60 (s, 1H), 4.52-4.36 (m, 2H), 3.80-3.51 (m, 6H), 3.42-3.29 (m, 3H), 3.27-3.03 (m, 5H), 2.91-2.78 (m, 2H), 2.37-2.23 (m, 2H), 2.01-1.85 (m, 1H), 1.79-1.60 (m, 1H), 1.54-1.33 (m, 2H).

### Example 14: Synthesis of Compound I-14

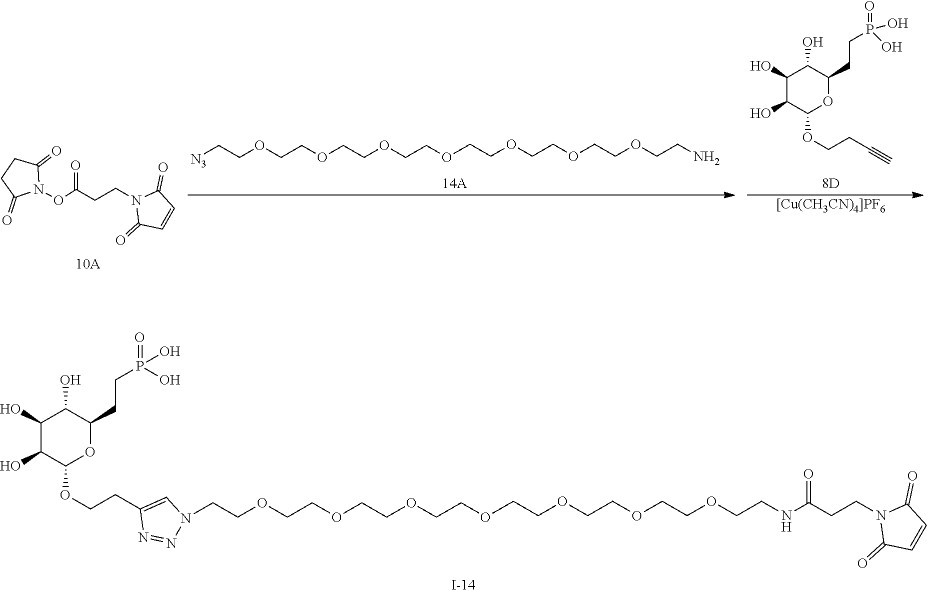

Compound I-14 was prepared using similar methods. Yield: 8.6 mg, 23%; LC-MS m/z 856.5 [M+1]+; ¹H NMR (300 MHz, DMSO-d₆ with D₂O) δ 8.04 (bs, 1H), 7.83 (s, 1H), 6.97 (s, 2H), 4.60 (s, 1H), 4.52-4.38 (m, 2H), 3.84-3.66 (m, 4H), 3.52-3.28 (m, 29H), 3.28-3.04 (m, 5H), 2.85 (t, J=6.7 Hz, 2H), 2.31 (t, J=7.4 Hz, 2H), 2.03-1.86 (m, 1H), 1.80-1.60 (m, 1H), 1.56-1.29 (m, 2H).

### Example 15: Synthesis of Compound I-15

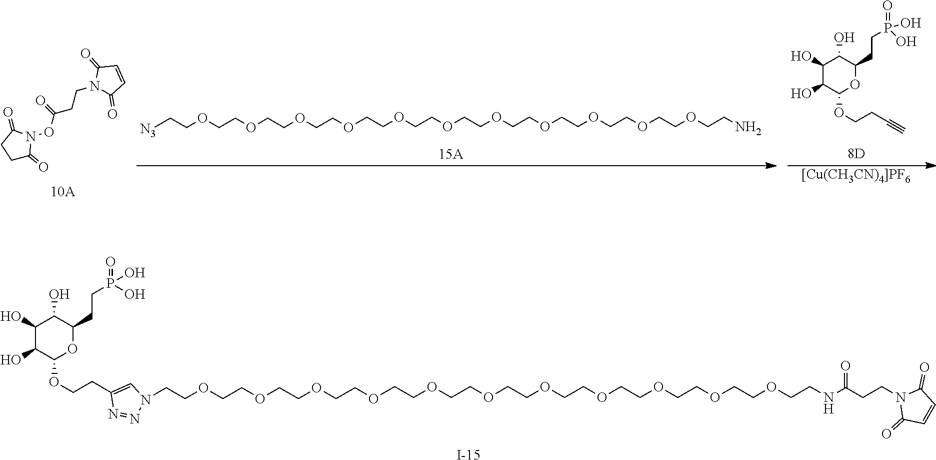

Compound I-15 was prepared using similar methods. Yield: 17.2 mg, 38%; LC-MS m/z 1032.6 [M+1]+; ¹H NMR (300 MHz, DMSO-d₆ with D₂O) δ 7.81 (s, 1H), 6.91 (s, 2H), 4.59 (s, 1H), 4.50-4.36 (m, 2H), 3.91-3.65 (m, 19H), 3.62-3.27 (m, 30H), 3.27-3.03 (m, 5H), 2.91-2.78 (m, 2H), 2.30 (t, J=7.4 Hz, 2H), 1.99-1.85 (m, 1H), 1.80-1.60 (m, 1H), 1.55-1.33 (m, 2H).

### Example 16: Synthesis of Compound I-16

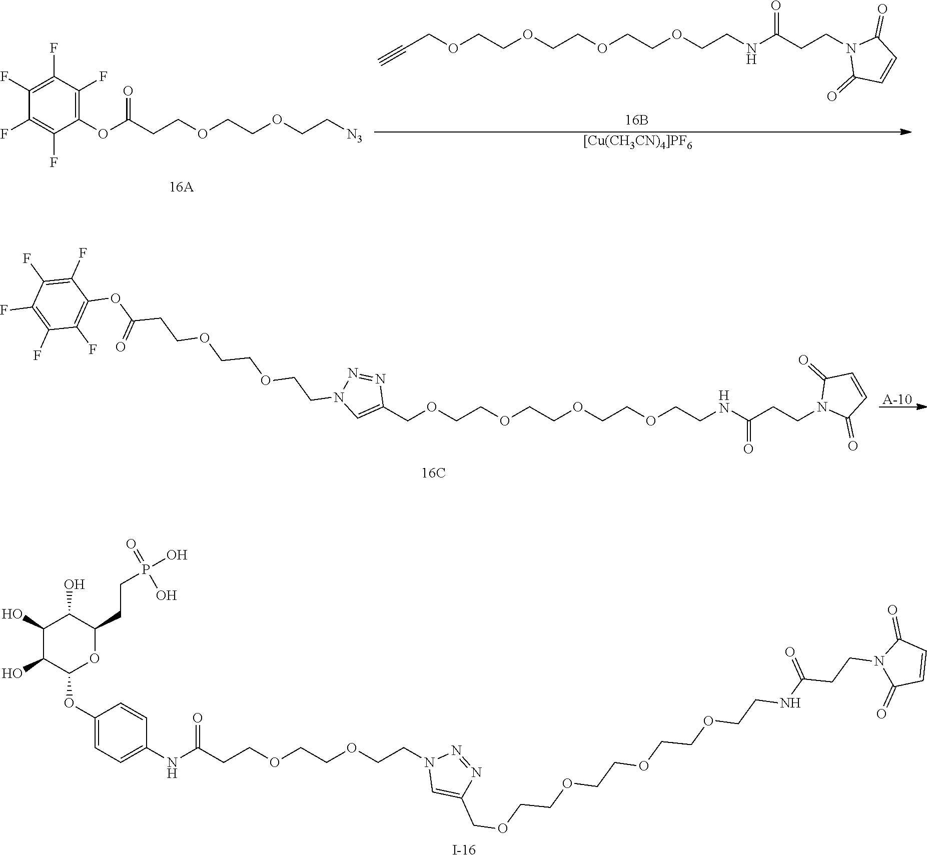

To a solution of perfluorophenyl 3-(2-(2-azidoethoxy)ethoxy)propanoate (16A) (1.0 eq, 0.200 g, 0.542 mmol) in dimethylsulfoxide (4 mL), 3-(2,5-dioxo-2,5-dihydro-1H-pyrrol-1-yl)-N-(3,6,9,12-tetraoxapentadec-14-yn-1-yl)propanamide (16B) (1.5 eq, 0.311 g, 0.812 mmol) was added and stirred for 5 minutes, then tetrakis(acetonitrile)copper(I) hexafluorophosphate (2.8 eq, 0.565 g, 1.52 mmol) was added and reaction mixture was stirred at room temperature for 1 h. After completion, reaction mixture was diluted with acetonitrile and purified by prep HPLC (45-75% acetonitrile in water with 0.1% TFA). Fractions containing the desired product were combined and lyophilized to dryness to afford Compound 16C as a colourless viscous liquid. Yield: 0.045 g, 10.88%; LC-MS m/z 752.33 [M+1]⁺.

In dimethylsulfoxide (0.6 mL), molecular sieves (Powder, Catalyst support, sodium Y zeolite, Aldrich Cat no 334448) was added followed by Intermediate A-10 (1.0 eq, 0.019 g, 0.054 mmol), triethylamine (3.0 eq, 0.023 mL, 0.163 mmol) and Compound 16C (1.1 eq, 0.045 g, 0.059 mmol) were added and reaction mixture was stirred at room temperature for 3 h. After completion, reaction mixture was diluted with acetonitrile and purified by prep HPLC (13-23% acetonitrile in water with 0.1% TFA). Fractions containing the desired product were combined and lyophilized to dryness to afford Compound I-16 as an off white solid. Yield: 0.008 g, 15.82%; LC-MS m/z 917.33 [M+1]; ¹H NMR (400 MHz, D₂O) δ 7.98 (s, 1H), 7.37 (d, J=8.8 Hz, 2H), 7.13 (d, J=8.8 Hz, 2H), 6.83 (s, 2H), 5.55 (s, 1H), 4.61 (s, 2H), 4.56-4.54 (m, 2H), 4.17-4.16 (m, 1H), 4.00-3.98 (m, 1H), 3.94 (t, J=4.8 Hz, 2H), 3.82-3.75 (m, 4H), 3.68-3.58 (m, 18H), 3.53 (t, J=5.2 Hz, 2H), 3.29 (t, J=5.6 Hz, 2H), 2.64 (t, J=6.0 Hz, 2H), 2.48 (t, J=6.4 Hz, 2H), 2.15-1.90 (m, 1H), 1.80-1.60 (m, 2H), 1.40-1.25 (m, 1H).

### Example 17: Synthesis of Compound I-17

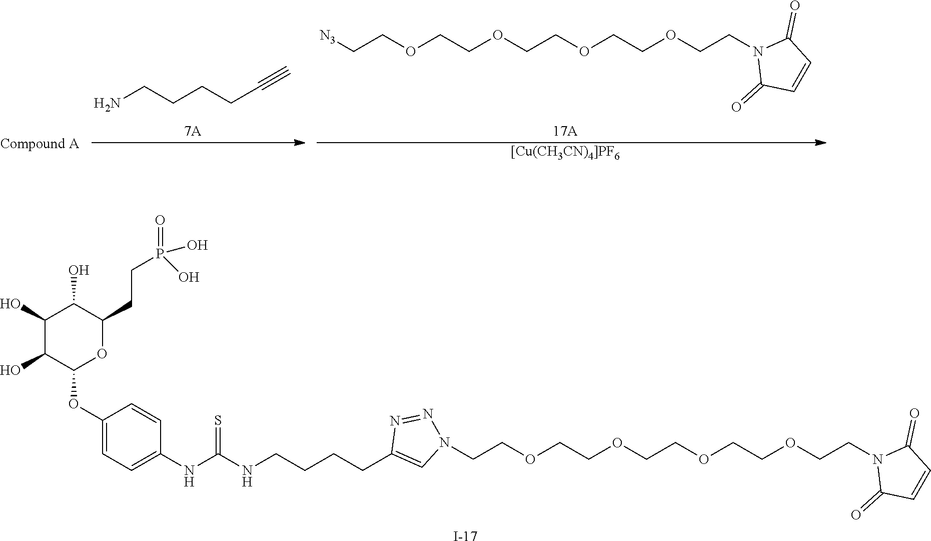

Compound I-17 is synthesized employing the procedures described for Compound I-7 using 1-(14-azido-3,6,9,12-tetraoxatetradecyl)-1H-pyrrole-2,5-dione (17A) in lieu of Compound 7B.

### Example 18: Synthesis of Compound I-18

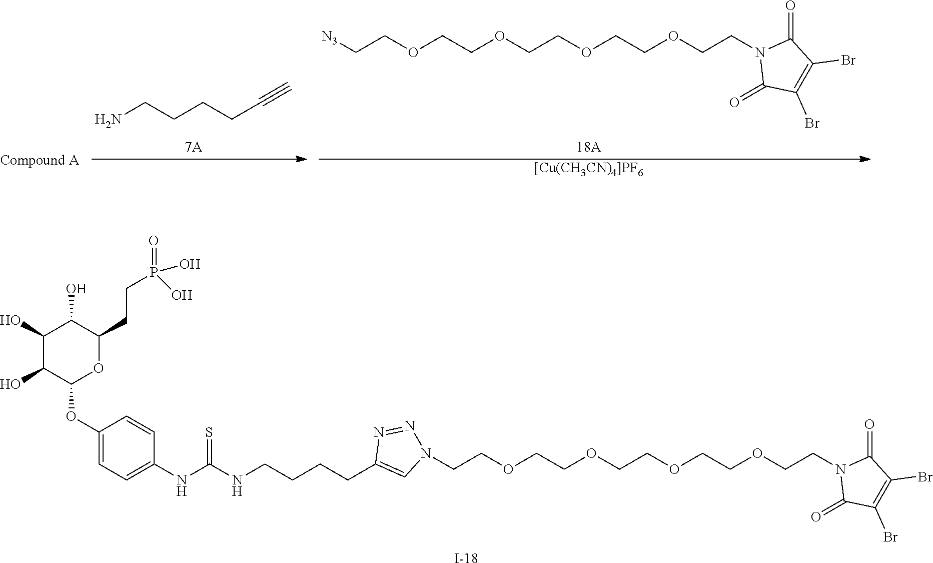

Compound I-18 is synthesized employing the procedures described for Compound I-7 using 1-(14-azido-3,6,9,12-tetraoxatetradecyl)-3,4-dibromo-1H-pyrrole-2,5-dione (18A) in lieu of Compound 7B.

### Example 19: Synthesis of Intermediates X-A, X-B, and X-C

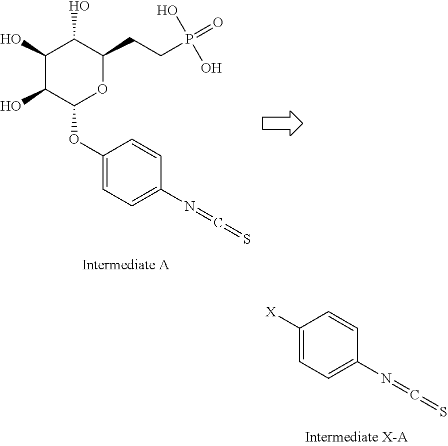

Intermediate X-A are synthesized employing the procedures described for Intermediate A using Compound X-H as the starting material in lieu of mannose 6-phosphate.

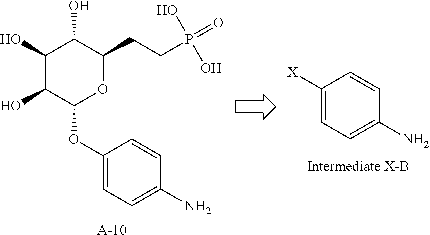

Intermediate X-B are synthesized employing the procedures described for Intermediate A-10 using X-H as the starting material in lieu of mannose 6-phosphate.

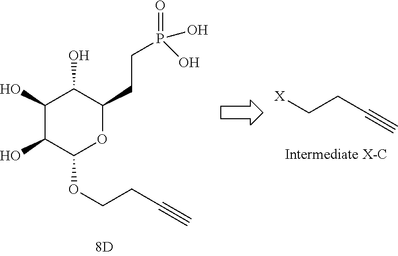

Intermediate X-C are synthesized employing the procedures described for Compound 8D using X-H as the starting material in lieu of mannose 6-phosphate.

### Example 20: Synthesis of Compound I-20

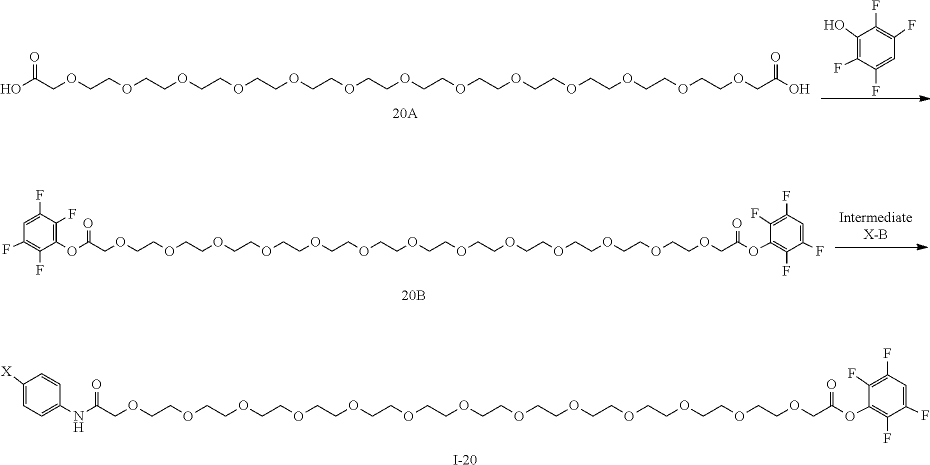

Compound 20B is synthesized by employing the procedure described for Compound 1B using Compound 20A in lieu of Compound 1A.

Compound I-20 is synthesized by employing the procedure described for Compound 1 using Compound 20B and Intermediate X-B in lieu of Compound 1B and Intermediate A-10.

### Example 21: Synthesis of Compound I-21

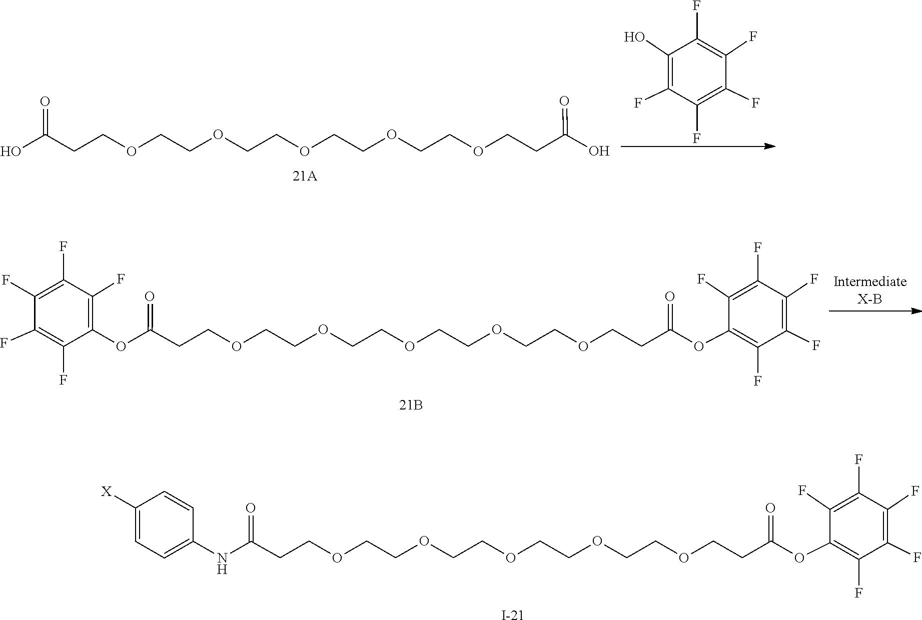

Compound 21B is synthesized by employing the procedure described for Compound 1B using Compound 21A and pentafluorophenol in lieu of Compound 1A and 2,3,5,6-tetrafluorophenol.

Compound I-21 is synthesized by employing the procedure described for Compound 1 using Compound 21B and Intermediate X-B in lieu of Compound 1B and Intermediate A-10.

### Example 22

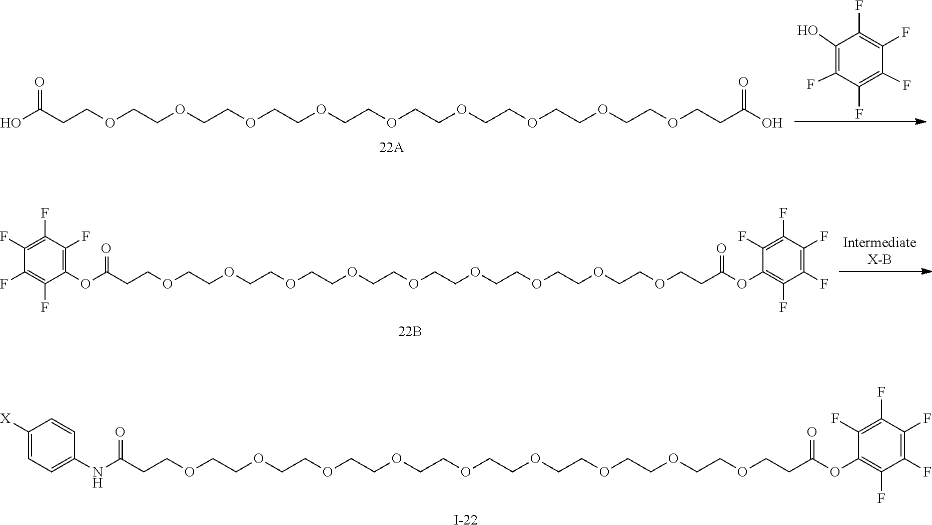

Compound 22B is synthesized by employing the procedure described for Compound 1B using Compound 22A and pentafluorophenol in lieu of Compound 1A and 2,3,5,6-tetrafluorophenol.

Compound I-22 is synthesized by employing the procedure described for Compound 1 using Compound 22B and Intermediate X-B in lieu of Compound 1B and Intermediate A-10.

### Example 23: Synthesis of Compound I-21

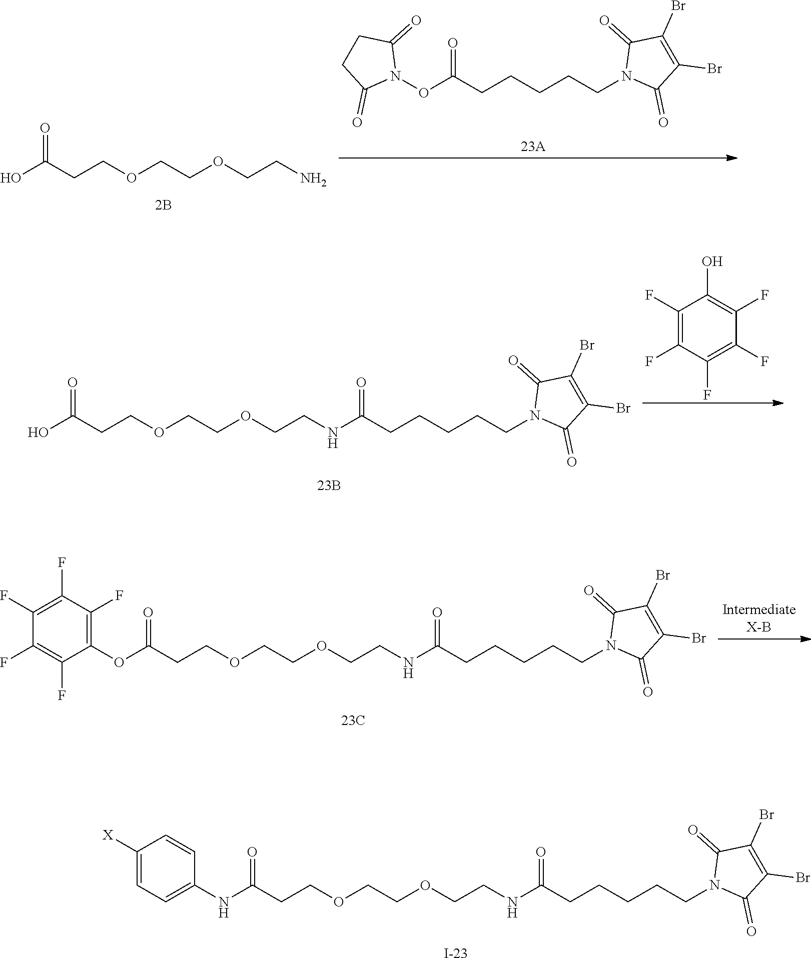

Compounds 23B and 23C are synthesized by employing the procedures described for Compound 2D and 2E using Compounds 23A and 23B in lieu of Compounds 2C and 2D.

Compound I-23 is synthesized by employing the procedure described for Compound 2 using Compound 23C and Intermediate X-B in lieu of Compound 2E and Intermediate A-10.

### Example 24: Synthesis of Compound I-24

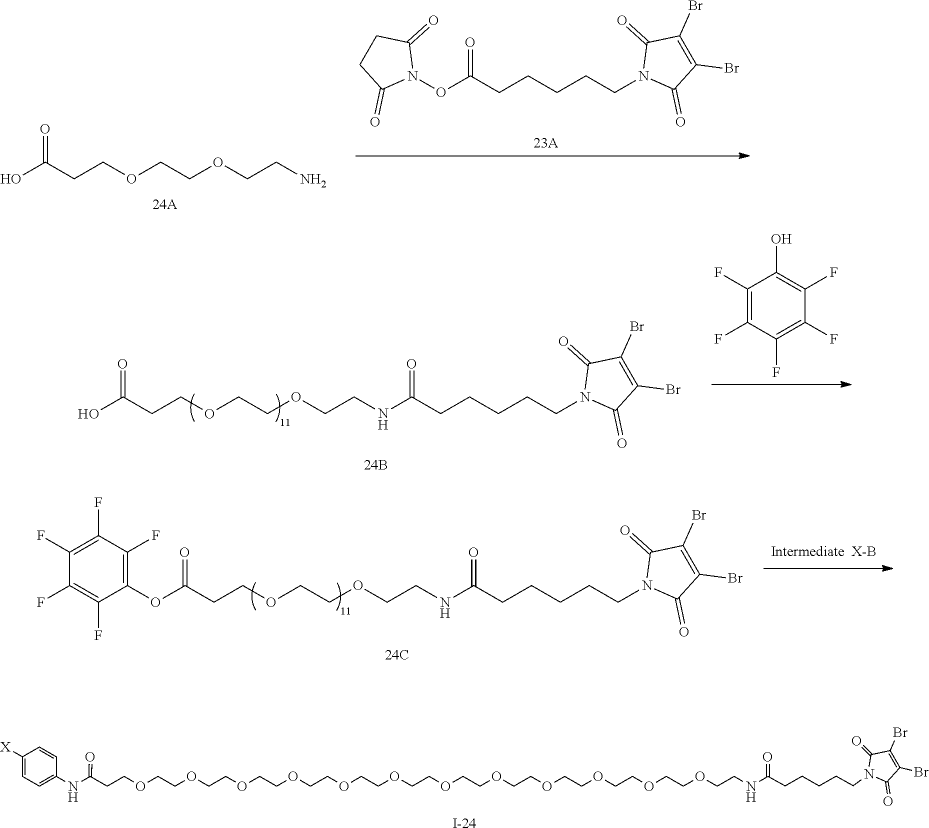

Compounds 24B and 24C are synthesized by employing the procedures described for Compound 2D and 2E using Compounds 24A, 23A and 24B in lieu of Compounds 2B, 2C and 2D.

Compound I-24 is synthesized by employing the procedure described for Compound 2 using Compound 24C and Intermediate X-B in lieu of Compound 2E and Intermediate A-10.

### Example 25: Synthesis of Compound 1-25

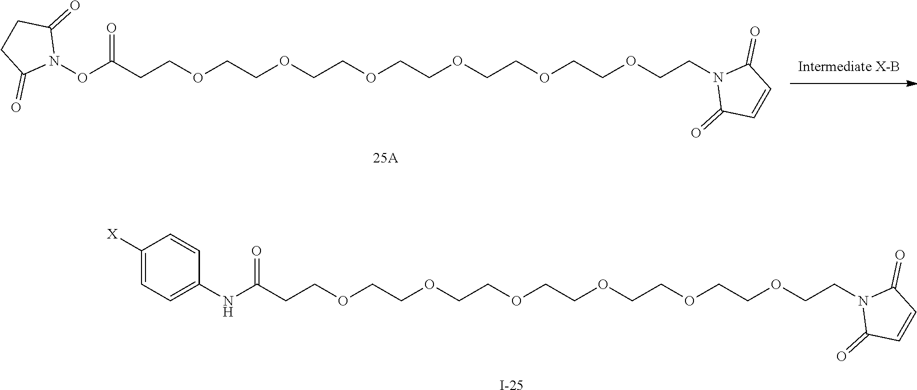

Compound I-25 is synthesized by employing the procedure described for Compound I-6 using Compound 25A and Intermediate X-B in lieu of Compound 6A and Intermediate A-10.

### Example 26: Synthesis of Compound I-26

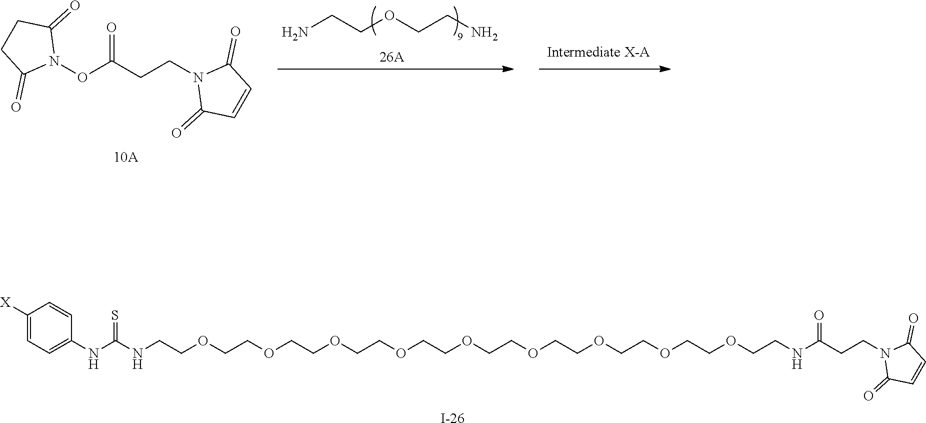

Compound I-26 is synthesized by employing the procedure described for Compound I-13 using Compound 26A and Intermediate X-A in lieu of Compounds 13A, 8D and tetrakis(acetonitrile)copper(I) hexafluorophosphate.

### Example 27: Synthesis of Compound I-27

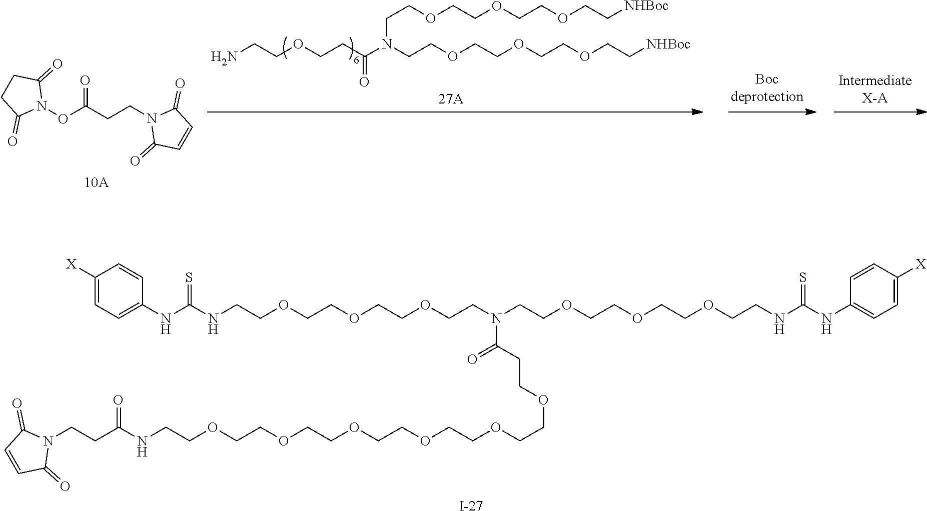

Compound I-27 is synthesized by employing the procedure described for Compound I-13 using Compound 27A and Intermediate X-A in lieu of Compounds 13A, 8D and tetrakis(acetonitrile)copper(I) hexafluorophosphate. A deprotection of the Boc protection group is performed under the standard Boc deprotection conditions before Intermediate X-A is added.

### Example 28: Synthesis of Compound I-28

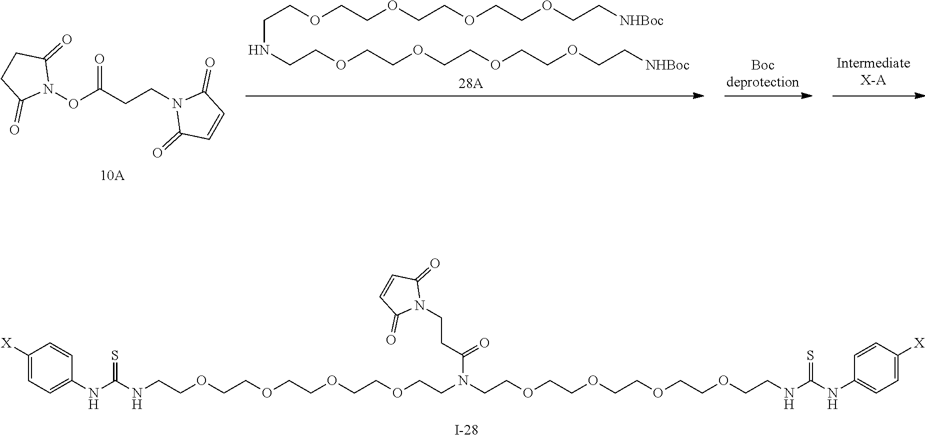

Compound I-28 is synthesized by employing the procedure described for Compound I-13 using Compound 28A and Intermediate X-A in lieu of Compounds 13A, 8D and tetrakis(acetonitrile)copper(I) hexafluorophosphate. A deprotection of the Boc protection group is performed under the standard Boc deprotection conditions before Intermediate X-A is added.

### Example 29: Synthesis of Compound I-28

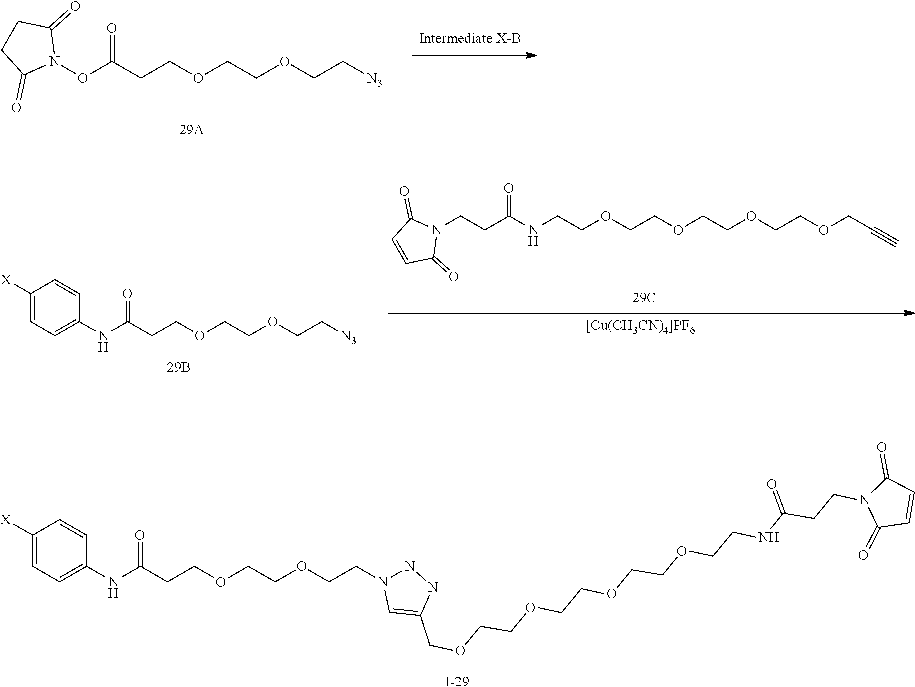

Compound 29B is synthesized by employing the procedure described for Compound 5B using Compound 29A and Intermediate X-B in lieu of Compound 5A and Intermediate A-10.

Compound I-29 is synthesized by employing the procedure described for Compound I-5 using Compounds 29B and 29C in lieu of Compounds 5B and 5C.

### Example 30: Synthesis of Compound I-30

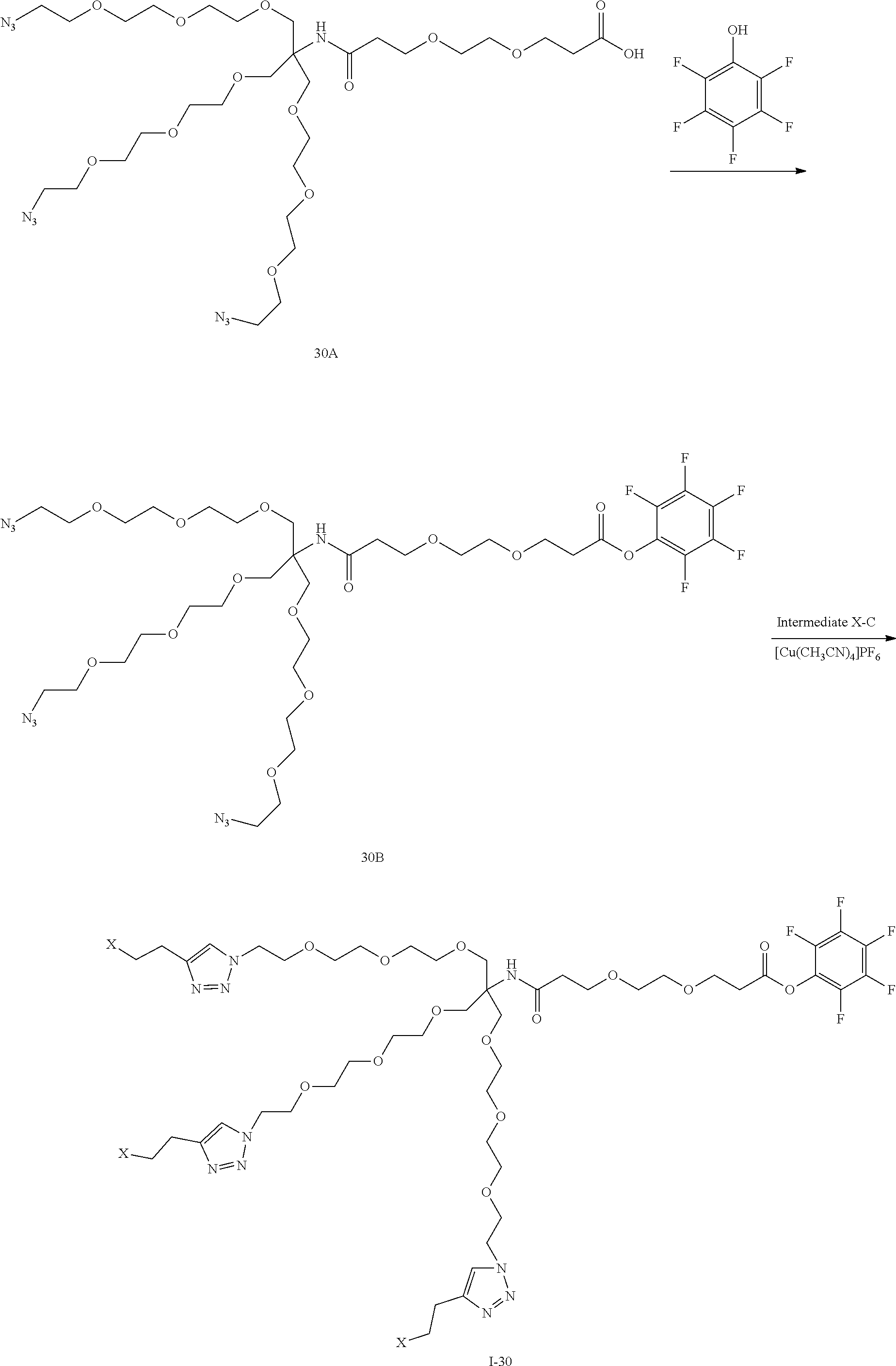

Compound 30B is synthesized by employing the procedure described for Compound 12B using Compound 30A in lieu of Compound 12A.

Compound I-30 is synthesized by employing the procedure described for Compound I-12 using Compound 30B and Intermediate X-C in lieu of Compounds 12B and 8D.

### Example 31

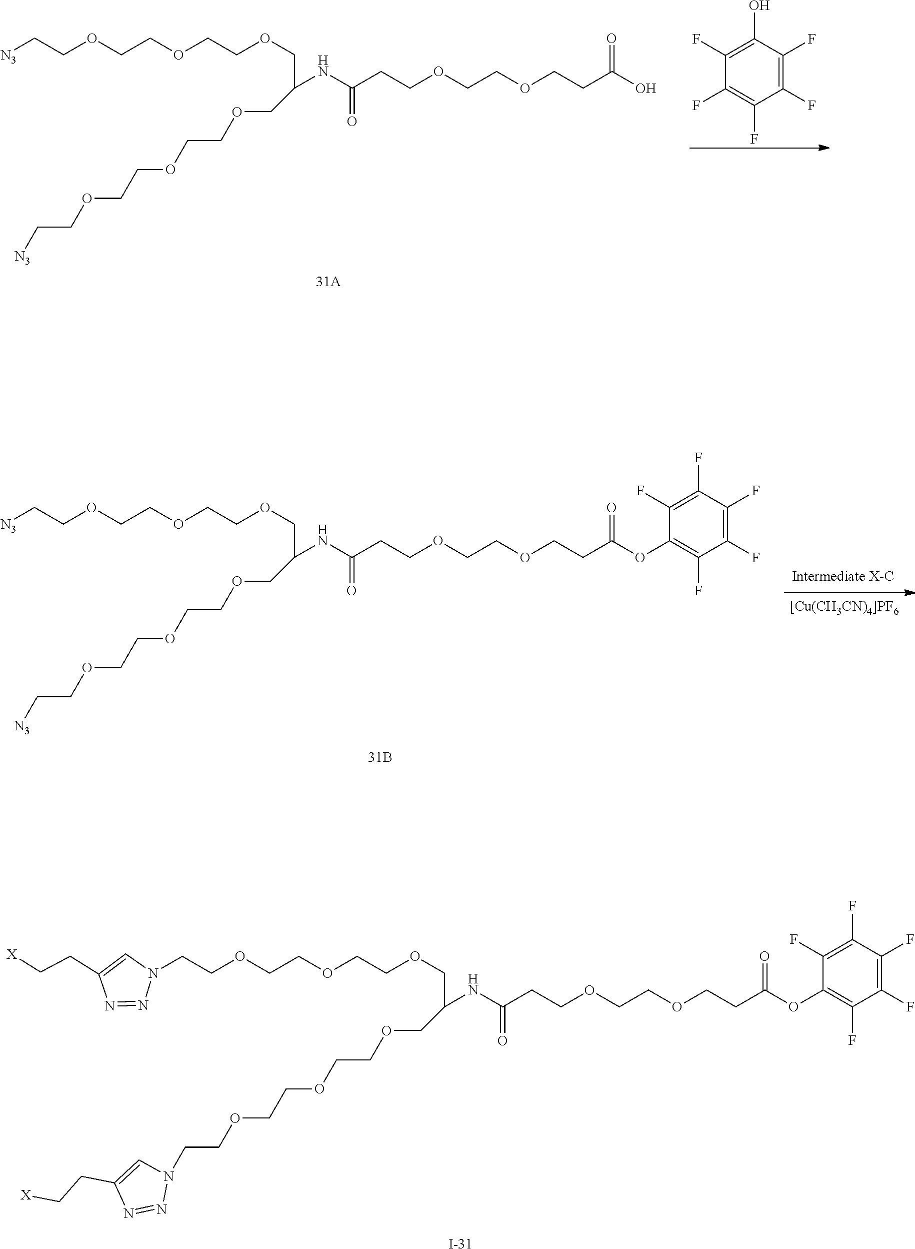

Compound 31B is synthesized by employing the procedure described for Compound 12B using Compound 31A in lieu of Compound 12A.

Compound I-31 is synthesized by employing the procedure described for Compound I-12 using Compound 31B and Intermediate X-C in lieu of Compounds 12B and 8D.

### Example 32: Synthesis of Compound I-32

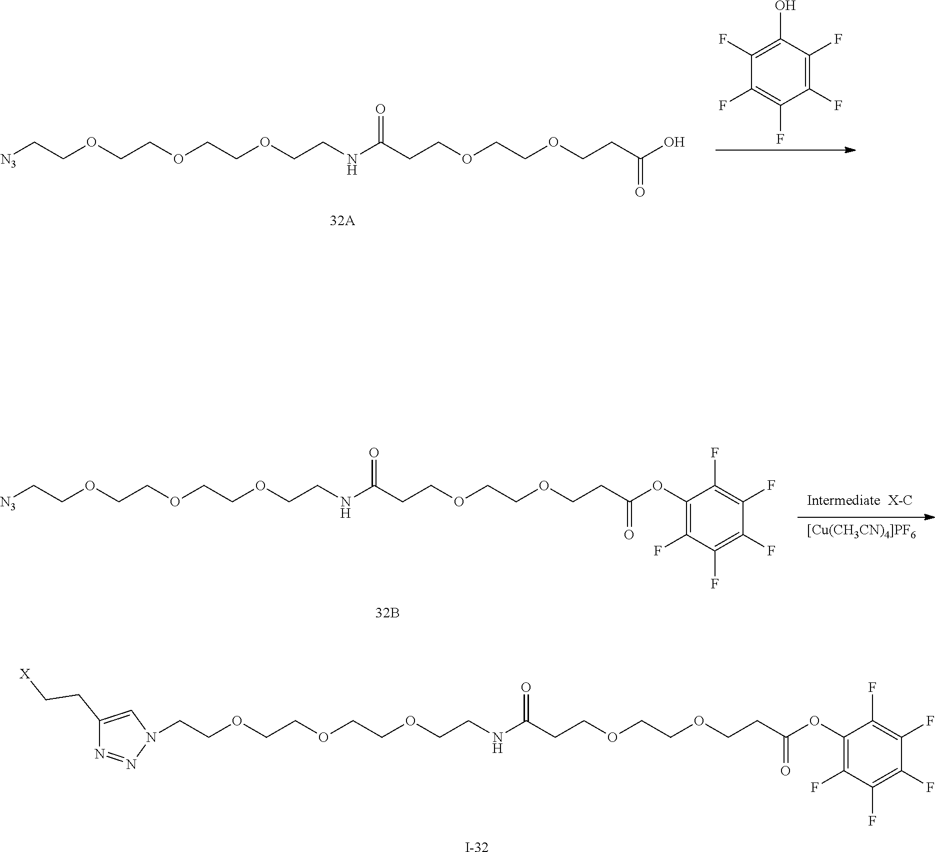

Compound 32B is synthesized by employing the procedure described for Compound 12B using Compound 32A in lieu of Compound 12A.

Compound I-32 is synthesized by employing the procedure described for Compound I-12 using Compound 32B and Intermediate X-C in lieu of Compounds 12B and 8D.

### Example 33: Synthesis of Compound I-33

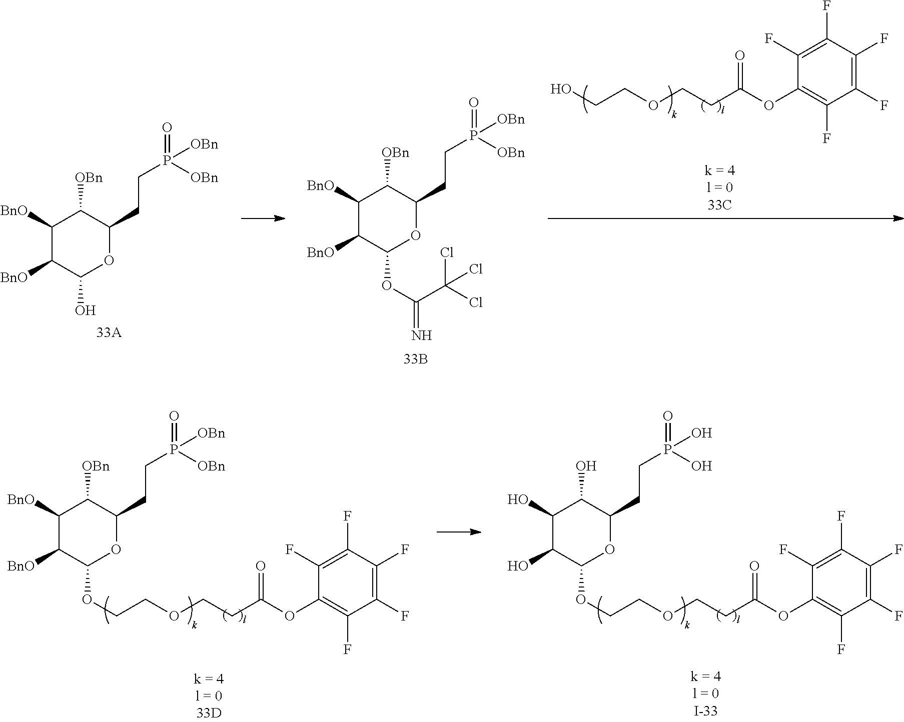

DBU (0.1 eq) is added to a stirred solution of dibenzyl (2-((2R,3R,4S,5S,6S)-3,4,5-tris(benzyloxy)-6-hydroxytetrahydro-2H-pyran-2-yl)ethyl)phosphonate (33A) (1.00 eq) and trichloroacetonitrile (10.0 eq) in DCM at 0° C. under nitrogen. The resulting mixture is stirred at 0° C. under nitrogen until LC-MS indicates complete conversion to Compound 33B. Most of the solvent is removed on a rotary evaporator. The residue is purified via silica gel chromatography to afford Compound 33B. Compound 33B (1.00 eq) is dissolved in dry DCM with stirring under nitrogen. Perfluorophenyl 14-hydroxy-3,6,9,12-tetraoxatetradecanoate (33C) (2.00 eq) is added and the resulting mixture is cooled to −78° C. with stirring under nitrogen. A solution of boron trifluoride diethyl etherate (0.500 eq) in dichloromethane is added slowly. The −78° C. cold bath is removed and the reaction mixture is allowed to slowly warm to 0° C. under nitrogen and then worked up. The crude material is purified via silica gel chromatography to afford Compound 33D. Compound 33D (1 eq) is dissolved with stirring in dry ethyl acetate. Palladium on carbon (0.05 eq) is added and the resulting mixture is stirred vigorously under a hydrogen balloon until LC-MS indicates complete conversion to Compound I-33. The resulting mixture is filtered through Celite, concentrated on a rotary evaporator, and purified via reverse phase preparatory HPLC to afford Compound I-33.

### Example 33: Synthesis of Compound B (See Above)

### Example 34: Synthesis of Compound I-34

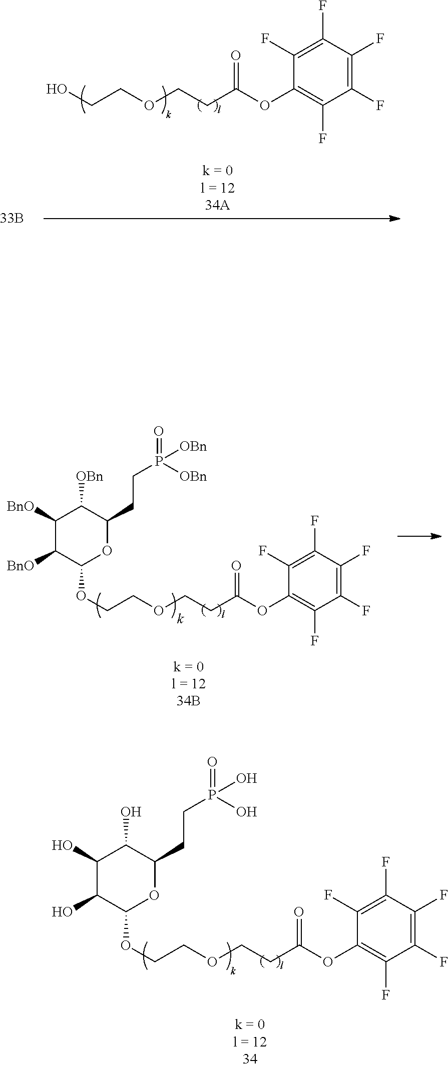

### Example 35: Synthesis of Compound I-35

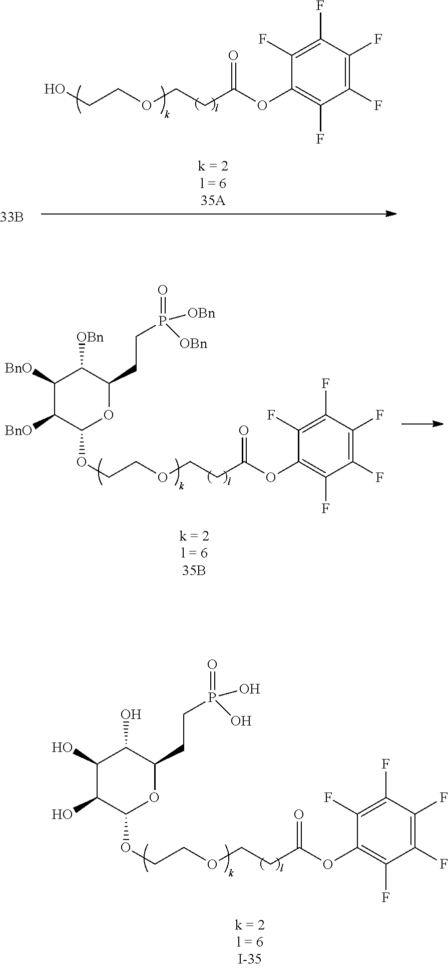

### Example 37: Synthesis of Compound I-37

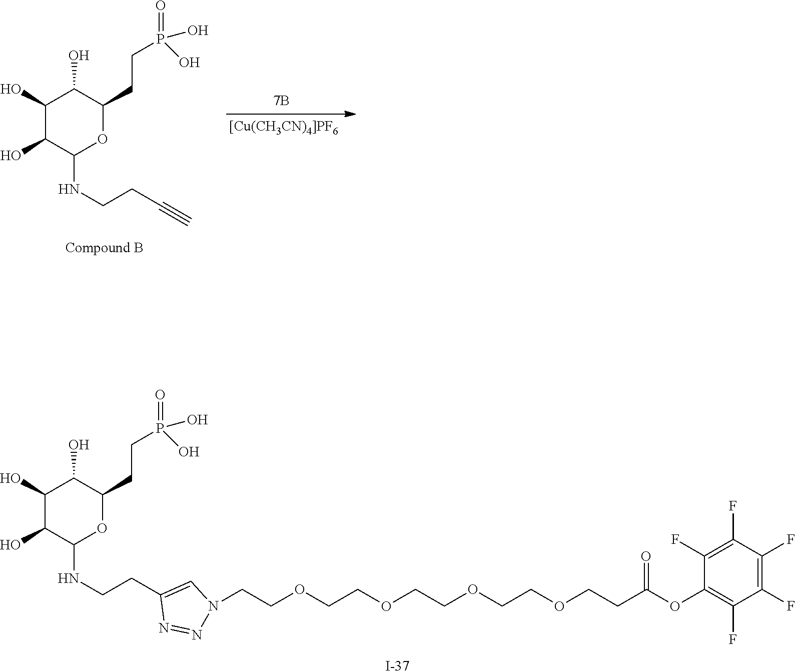

Compound I-37 is synthesized employing the procedures described for Compound I-8 using Compound B in lieu of Compound 8D.

### Example 38: Synthesis of Compound I-38

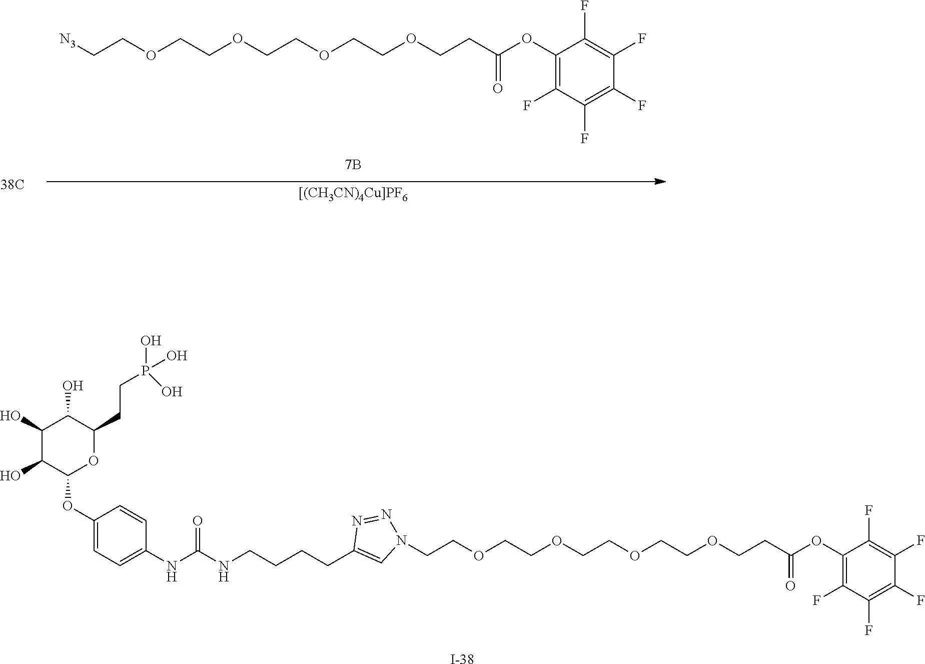

Compound I-38 was prepared using similar methods. Yield: 12 mg, 37%; LCMS m/z 930.5 [M+1]+; ¹H NMR (300 MHz, DMSO-d6 D₂O) δ 7.77 (s, 1H), 7.24 (d, J=8.5 Hz, 2H), 6.88 (d, J=8.6 Hz, 2H), 5.23 (s, 1H), 4.42 (t, J=5.1 Hz, 2H), 3.89-3.25 (m, 20H), 3.05 (t, J=6.2 Hz, 2H), 2.95 (t, J=5.8 Hz, 2H), 2.59 (t, J=7.5 Hz, 2H), 2.02-1.82 (m, 1H), 1.70-1.33 (m, 6H), 1.30-1.05 (m, 1H).

### Example 39: Synthesis of Compound I-39

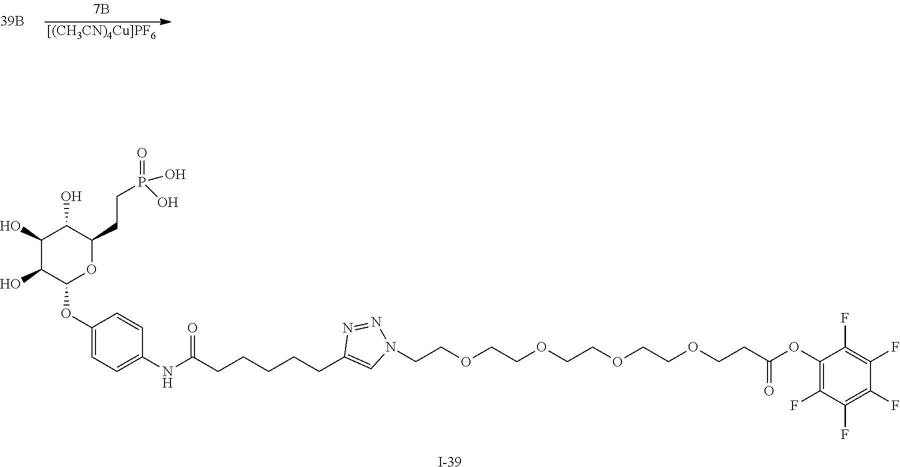

Compound I-39 was prepared using similar methods. Yield: 55 mg, 59%; LCMS m/z 929.6 [M+1]+; ¹H NMR (300 MHz, DMSO-d6 with D₂O) δ 7.76 (s, 1H), 7.46 (d, J=8.8 Hz, 2H), 6.94 (d, J=8.2 Hz, 2H), 5.28 (s, 1H), 4.41 (t, J=5.1 Hz, 2H), 3.86-2.87 (m, 22H), 2.64-2.53 (m, 2H), 2.23 (t, J=7.5 Hz, 2H), 1.99-1.80 (m, 1H), 1.68-1.40 (m, 6H), 1.37-1.05 (m, 3H).

### Example 40: Synthesis of Compound I-40

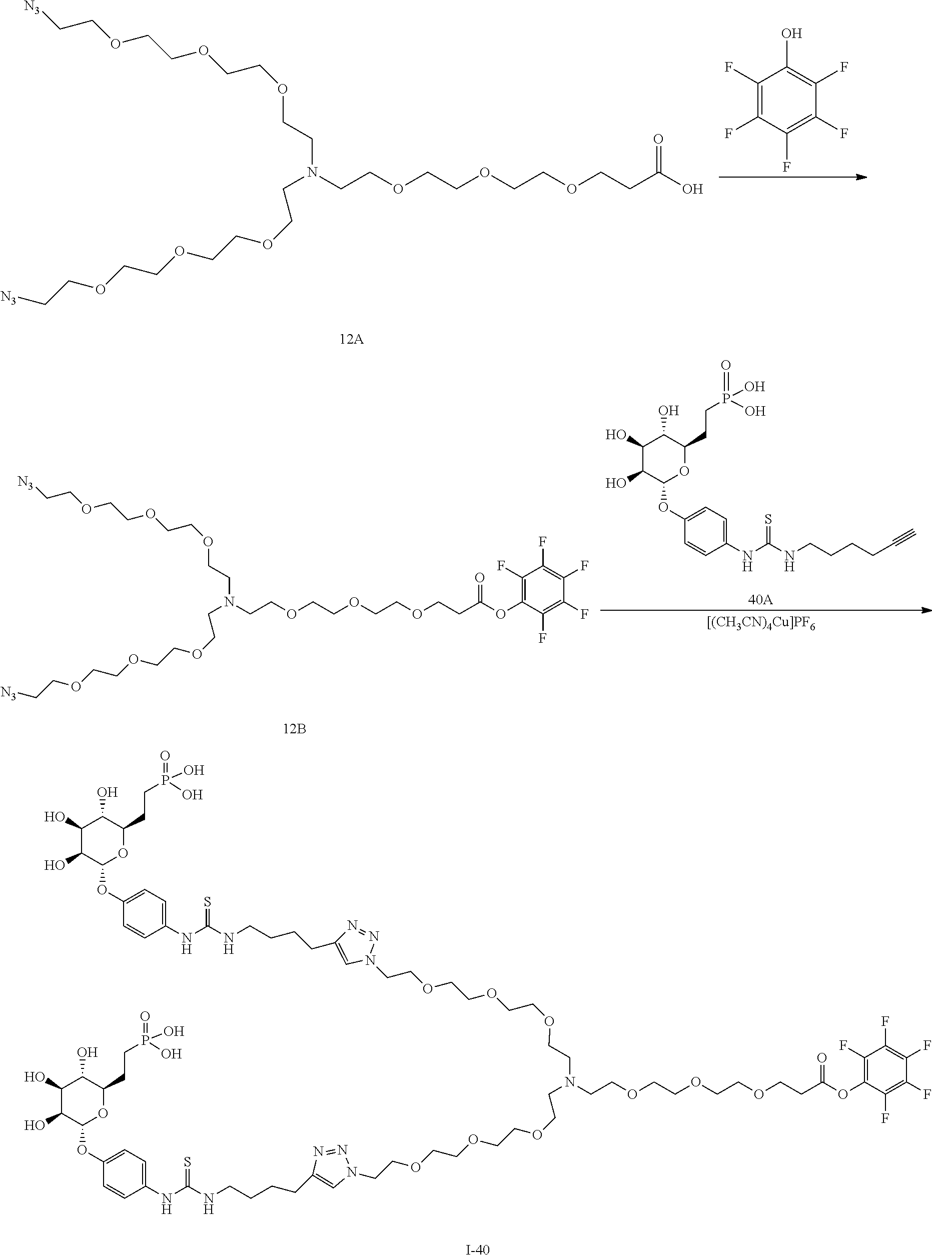

Compound I-40 was prepared using similar methods. Yield: 38.9 mg, 66%; LCMS m/z 1765.9 [M−1]-; ¹H NMR (300 MHz, DMSO-d₆ with D₂O) δ 7.81 (s, 2H), 7.19 (d, J=8.5 Hz, 4H), 6.99 (d, J=8.8 Hz, 4H), 5.33 (s, 2H), 4.43 (t, J=5.2 Hz, 4H), 3.90-3.23 (m, 54H), 2.97 (t, J=5.8 Hz, 2H), 2.69-2.34 (m, 4H), 2.01-1.81 (m, 2H), 1.73-1.40 (m, 12H), 1.34-1.10 (m, 2H).

### Example 41: Synthesis of Compound I-41

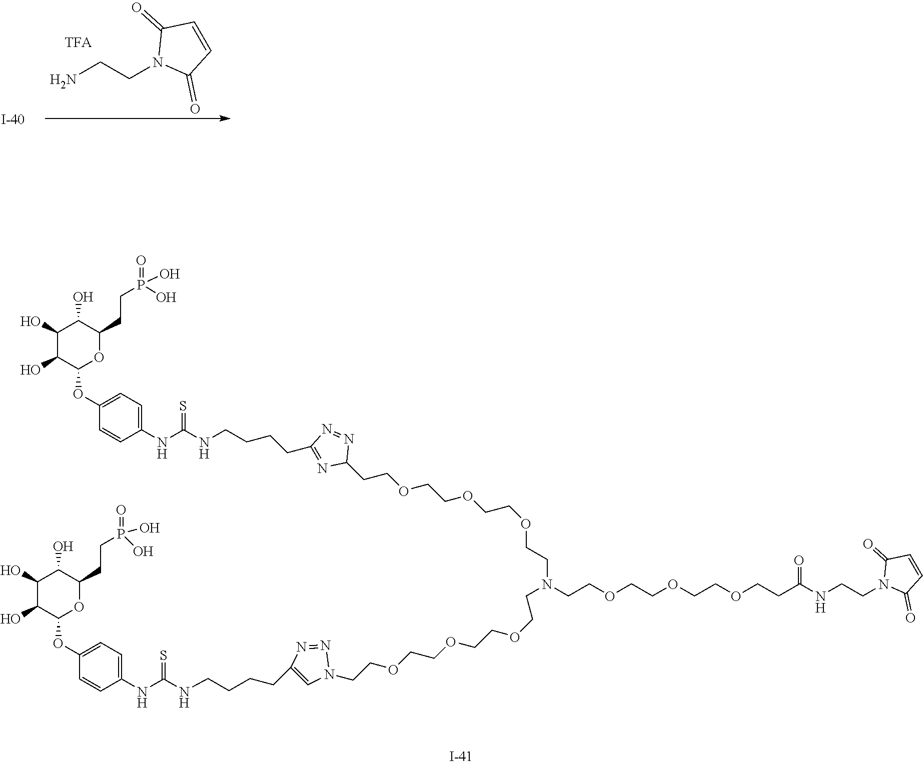

Compound I-41 was prepared using similar methods. Yield: 18.5 mg, 58%; LCMS m/z 1722.0 [M−1]-; ¹H NMR (300 MHz, DMSO-d₆ with D₂O) δ 7.82 (s, 2H), 7.26-7.09 (m, 4H), 7.00 (d, J=8.5 Hz, 4H), 6.90 (s, 2H), 5.33 (s, 2H), 4.52-4.32 (m, 4H), 3.99-2.94 (m, 58H), 2.69-2.57 (m, 4H), 2.20 (t, J=6.5 Hz, 2H), 1.99-1.81 (m, 2H), 1.73-1.39 (m, 12H), 1.33-1.08 (m, 2H).

### Example 42: Synthesis of Compound I-42

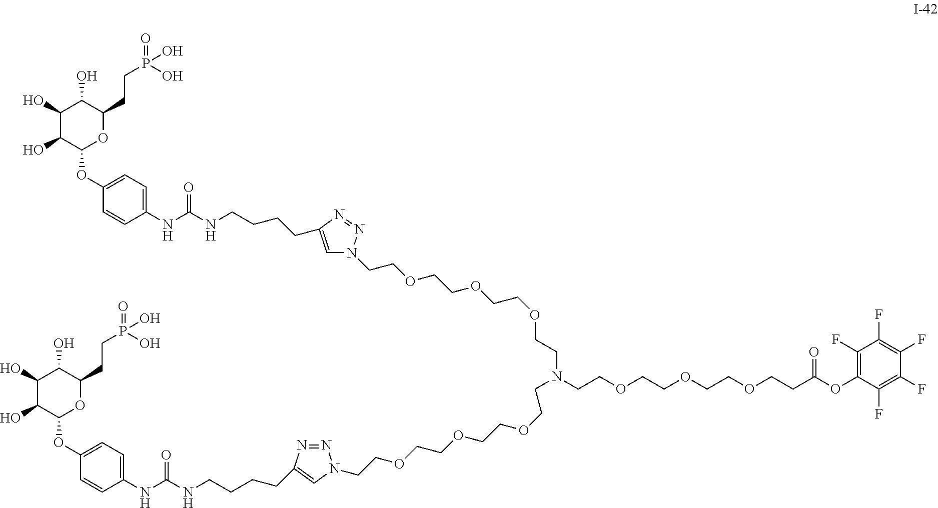 [SMILES: O=C(NCCCCc1cn(CCOCCOCCOCCN(CCOCCOCCOCCC(=O)Oc2c(F)c(F)c(F)c(F)c2F)CCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)nn1)Nc1ccc(O[C@H]2O[C@H](CCP(=O)(O)O)[C@@H](O)[C@H](O)[C@@H]2O)cc1]

Compound I-42 was prepared from 12B and 38C using similar methods. Yield: 40.0 mg, 61%; LCMS m/z 1734.0 [M−1]-; H NMR (300 MHz, DMSO-d₆ with D₂O) δ 7.77 (s, 2H), 7.24 (d, J=8.5 Hz, 4H), 6.88 (d, J=8.6 Hz, 4H), 5.23 (s, 2H), 4.42 (t, J=5.1 Hz, 4H), 3.92-3.23 (m, 48H), 3.05 (t, J=6.2 Hz, 4H), 2.95 (t, J=5.8 Hz, 4H), 2.59 (t, J=7.5 Hz, 4H), 2.03-1.81 (m, 2H), 1.68-1.33 (m, 12H), 1.29-1.07 (m, 2H).

### Example 43: Synthesis of Compound I-43

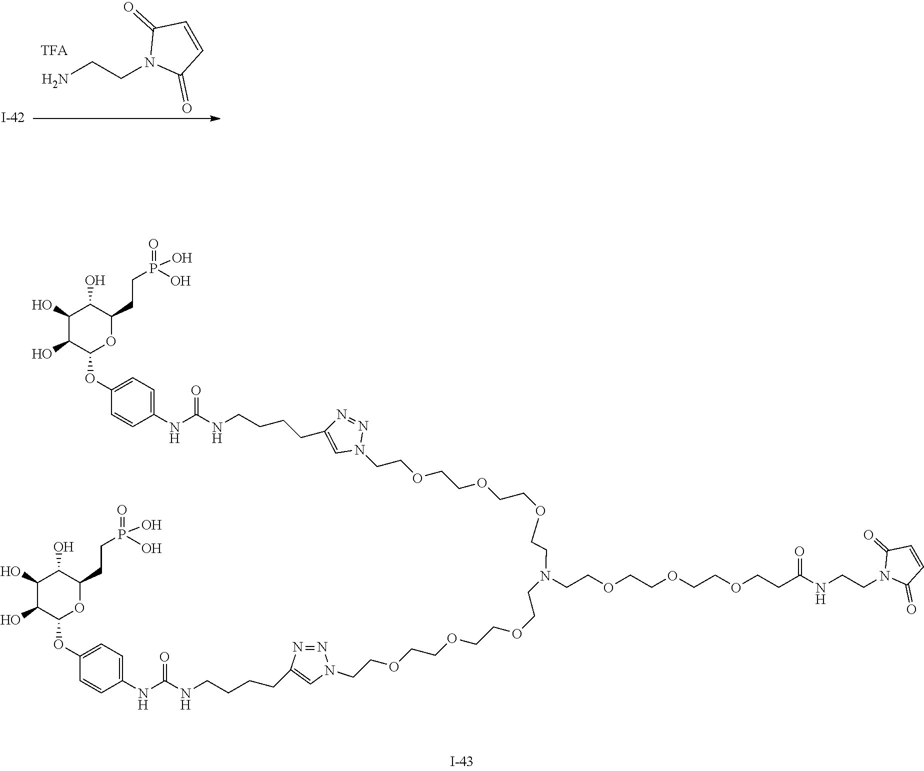

Compound I-43 was prepared using similar methods. Yield: 17.0 mg, 67%; LCMS m/z 1690.0 [M−1]-; ¹H NMR (300 MHz, DMSO-d₆ with D₂O) δ 7.76 (s, 2H), 7.23 (d, J=8.7 Hz, 4H), 6.97-6.80 (m, 6H), 5.24 (s, 2H), 4.48-4.33 (m, 4H), 4.04-2.95 (m, 58H), 2.59 (t, J=7.4 Hz, 4H), 2.19 (t, J=6.5 Hz, 2H), 2.01-1.81 (m, 2H), 1.69-1.32 (m, 12H), 1.32-1.07 (m, 2H).

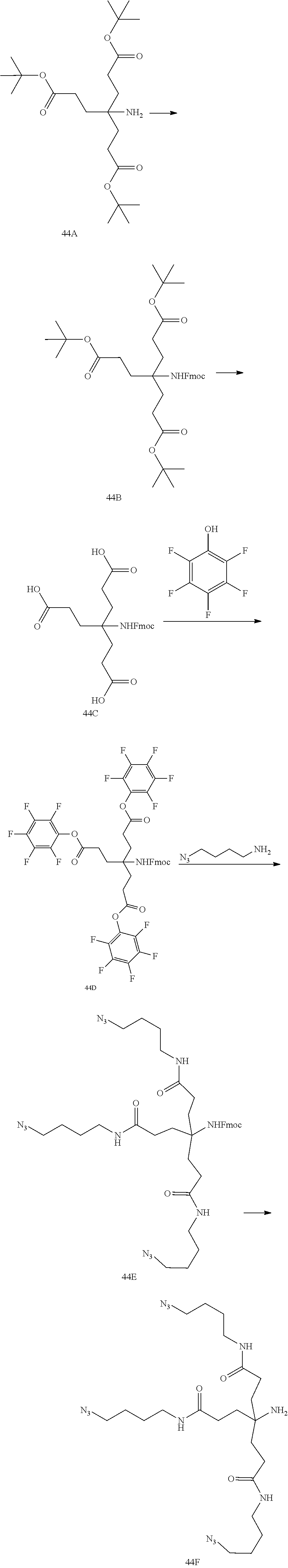

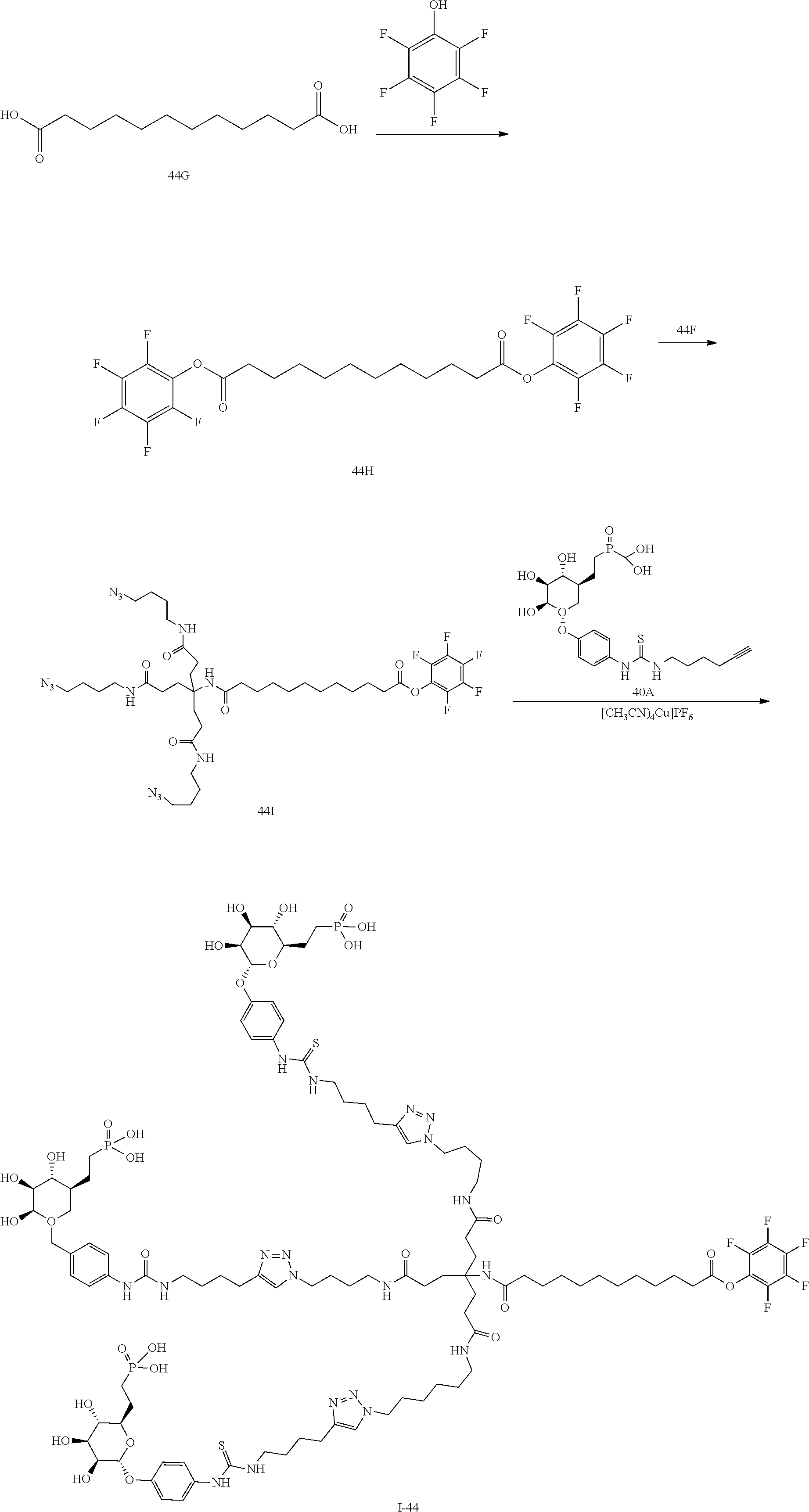

To a stirred mixture of di-tert-butyl 4-amino-4-(3-(tert-butoxy)-3-oxopropyl)heptanedioate (44A) (1.00 eq, 1.01 g, 2.43 mmol) in 1,4-dioxane (10 mL) at 0° C. was added 1 M sodium carbonate in water (1.50 eq, 3.6 mL, 3.65 mmol) and then a solution of FMOC-Cl (1.20 eq, 755 mg, 2.92 mmol) in 1,4-dioxane (4 mL). The cold bath was removed and the resulting mixture was stirred vigorously at room temperature for 2 h. The reaction mixture was partitioned between ethyl acetate and brine. The organics were dried over magnesium sulfate, filtered, concentrated on a rotary evaporator, and purified via silica gel chromatography (0-30% ethyl acetate in hexanes) to afford Compound 44B as a white foam-solid. Yield: 1.50 g, 97%; LCMS m/z 660.6 [M+Na]+; ¹H NMR (300 MHz, Chloroform-d) δ 7.76 (d, J=7.4 Hz, 2H), 7.59 (d, J=7.4 Hz, 2H), 7.40 (t, J=7.5 Hz, 2H), 7.31 (t, J=7.4 Hz, 2H), 5.01 (s, 1H), 4.36 (d, J=6.2 Hz, 2H), 4.18 (t, J=6.5 Hz, 1H), 2.25-2.12 (m, 6H), 1.98-1.83 (m, 6H), 1.43 (s, 27H).

To a stirred solution of Compound 44B (1.00 eq, 1.50 g, 2.35 mmol) in DCM (10 mL) at 0° C. was added water (0.5 mL) and then TFA (3 mL). The resulting mixture was allowed to warm to room temperature and then stirred at room temperature for 18 h. More TFA (2 mL) was added and stirring at room temperature was continued for another 26 h. Volatiles were removed on a rotary evaporator. The residue was concentrated to dryness twice from dry toluene and then dried under high vacuum to afford Compound 44C as a white solid. Yield: 1.19 g. LCMS 470.4 m/z [M+1]+; ¹H NMR (300 MHz, DMSO-d₆ with D₂O) δ 7.86 (d, J=7.5 Hz, 2H), 7.68 (d, J=7.5 Hz, 2H), 7.39 (t, J=7.4 Hz, 2H), 7.30 (t, J=7.9 Hz, 2H), 4.28-4.11 (m, 3H), 2.19-2.00 (m, 6H), 1.87-1.66 (m, 6H).

Compound 44C (1.00 eq, 549 mg, 1.17 mmol), 4-dimethylaminopyridine (0.0200 eq, 2.9 mg, 0.0234 mmol), DCC (3.30 eq, 796 mg, 3.86 mmol), pentafluorophenol (3.50 eq, 753 mg, 4.09 mmol), and DMF (2.5 mL) were combined in a scintillation vial with a stirbar, capped, and stirred at room temperature for 4 h. More DCC (482 mg, 2.34 mmol) and pentafluorophenol (430 mg, 2.34 mmol) in DMF (1 mL) was added and the resulting mixture was capped and stirred at room temperature for 2 h. The reaction mixture was diluted with diethyl ether and filtered. The filtrate was washed three times with brine, dried over magnesium sulfate, filtered, concentrated on a rotary evaporator, and purified via silica gel chromatography (0-50% ethyl acetate in hexanes) to afford Compound 44D and pentafluorophenol as a light yellow oil. Yield: 1.54 g. This material was taken on to the next step without further purification.

4-Azidobutan-1-amine (0.5 M in mTBE) (4.00 eq, 8.7 mL, 4.34 mmol) was added to a stirred solution of Compound 44D (1.00 eq, 1.50 g, 1.09 mmol) in THF (10 mL) at room temperature. The resulting clear solution was capped and stirred at room temperature for 2 h. Most of the volatiles were removed on a rotary evaporator at room temperature. The residue was loaded onto a silica gel loading column with DCM and purified via silica gel chromatography (0-100% ethyl acetate in DCM) then (0-10% methanol in DCM) to afford Compound 44E as a colorless waxy solid. Yield: 624 mg, 76%; LCMS m/z 758.6 [M+1]+; ¹H NMR (300 MHz, Chloroform-d) δ 7.77 (d, J=7.5 Hz, 2H), 7.60 (d, J=7.4 Hz, 2H), 7.41 (t, J=7.4 Hz, 2H), 7.31 (t, J=7.4 Hz, 2H), 6.08 (bs, 3H), 5.67 (bs, 1H), 4.37 (d, J=7.0 Hz, 2H), 4.18 (t, J=6.7 Hz, 1H), 3.34-3.13 (m, 12H), 2.24-2.09 (m, 6H), 2.04-1.85 (m, 6H), 1.66-1.47 (m, 12H).

Diethylamine (20.0 eq, 1.7 mL, 16.3 mmol) was added to a stirred solution of Compound 44E (1.00 eq, 619 mg, 0.817 mmol) in methanol (8 mL). The resulting clear solution was capped and stirred at room temperature for 16 h. Volatiles were removed on a rotary evaporator. Methanol (10 mL) was added and volatiles were removed on a rotary evaporator again. This was repeated again to drive off diethylamine. The residue was taken up in methanol and loaded onto a 5 g Strata X-C ion exchange column from Phenomenex. The column was eluted sequentially with acetonitrile, methanol, and then 5% ammonium hydroxide in methanol. Fractions containing the desired product were combined, concentrated on a rotary evaporator and dried under high vacuum to afford Compound 44F at 90% purity as a yellow oil. Yield: 483 mg, 99%; LCMS m/z 536.8 [M+1]+; ¹H NMR (300 MHz, Chloroform-d) δ 6.33 (t, J=5.8 Hz, 3H), 3.48 (s, 2H), 3.36-3.17 (m, 12H), 2.33-2.12 (m, 6H), 1.74-1.51 (m, 18H).

To a stirred solution of dodecanedioic acid (44G) (1.00 eq, 610 mg, 2.65 mmol) in THF (10 mL) under nitrogen was added sequentially: a solution of pentafluorophenol (2.50 eq, 1.22 g, 6.62 mmol) in THF (1 mL), EDC HCl (2.20 eq, 1.12 g, 5.83 mmol), and then DIPEA (2.50 eq, 1.2 mL, 6.62 mmol). The resulting white mixture was stirred at room temperature under nitrogen for 4 h. The reaction mixture was partitioned between ethyl acetate and 1 N HCl in water. The organics were washed twice with brine, dried over magnesium sulfate, filtered, concentrated on a rotary evaporator, and purified via silica gel chromatography (0-50% ethyl acetate in hexanes) to afford Compound 44H as a white solid. Yield: 1.04 g, 70%; ¹H NMR (300 MHz, Chloroform-d) δ 2.66 (t, J=7.4 Hz, 4H), 1.77 (p, J=7.2 Hz, 4H), 1.48-1.22 (m, 12H).

Compound 44F (1.00 eq, 66.3 mg, 0.111 mmol), Compound 44H (3.00 eq, 188 mg, 0.334 mmol), DIPEA (5.00 eq, 0.097 mL, 0.557 mmol), and 1,4-dioxane (0.2500 mL) were combined in a sealable vessel with a stirbar, sealed, stirred, and heated at 80° C. with a heating block for 30 min. After cooling to room temperature volatiles were removed on a rotary evaporator at 30° C. The residue was taken up in a mixture of NMP, ethanol, and acetic acid, filtered, and purified via preparatory HPLC (30-90% acetonitrile in water with 0.1% TFA). Fractions containing the desired product were combined and lyophilized to dryness to afford Compound 441 as a yellow waxy solid. Yield: 27.8 mg, 27%; LCMS m/z 914.7 [M+1]+; ¹H NMR (300 MHz, Chloroform-d) δ 7.53 (s, 1H), 6.46 (t, J=6.1 Hz, 3H), 3.38-3.13 (m, 12H), 2.66 (t, J=7.5 Hz, 2H), 2.35-2.11 (m, 8H), 2.09-1.94 (m, 6H), 1.84-1.69 (m, 2H), 1.66-1.51 (m, 14H), 1.44-1.22 (m, 12H).

Compound 40A (3.20 eq, 52.5 mg, 0.107 mmol), Compound 441 (1.00 eq, 30.7 mg, 0.0336 mmol), and NMP (0.6 mL) were combined in a 1 dram vial with a stirbar, capped and stirred at room temperature. After 5 min, [(CH₃CN)₄Cu]PF₆ (7.00 eq, 87.6 mg, 0.235 mmol) was added. The resulting light yellow solution was capped and stirred at room temperature for 1 h. The reaction mixture slowly turned more green-colored. The reaction mixture was diluted with a mixture of NMP and acetic acid, filtered, and purified via preparatory HPLC (20-60% acetonitrile in water with 0.1% TFA). Fractions containing the desired product were combined and lyophilized to dryness to afford Compound I-44 as a white solid. Yield: 29.1 mg, 36%; H NMR (300 MHz, DMSO-d₆ with D₂O) S 7.84 (s, 3H), 7.13 (d, J=8.5 Hz, 6H), 7.00 (d, J=8.4 Hz, 6H), 5.34 (s, 3H), 4.27 (bs, 6H), 3.72-2.37 (m, 42H), 2.10-1.00 (m, 56H).

### Example 45: Synthesis of Compound I-45

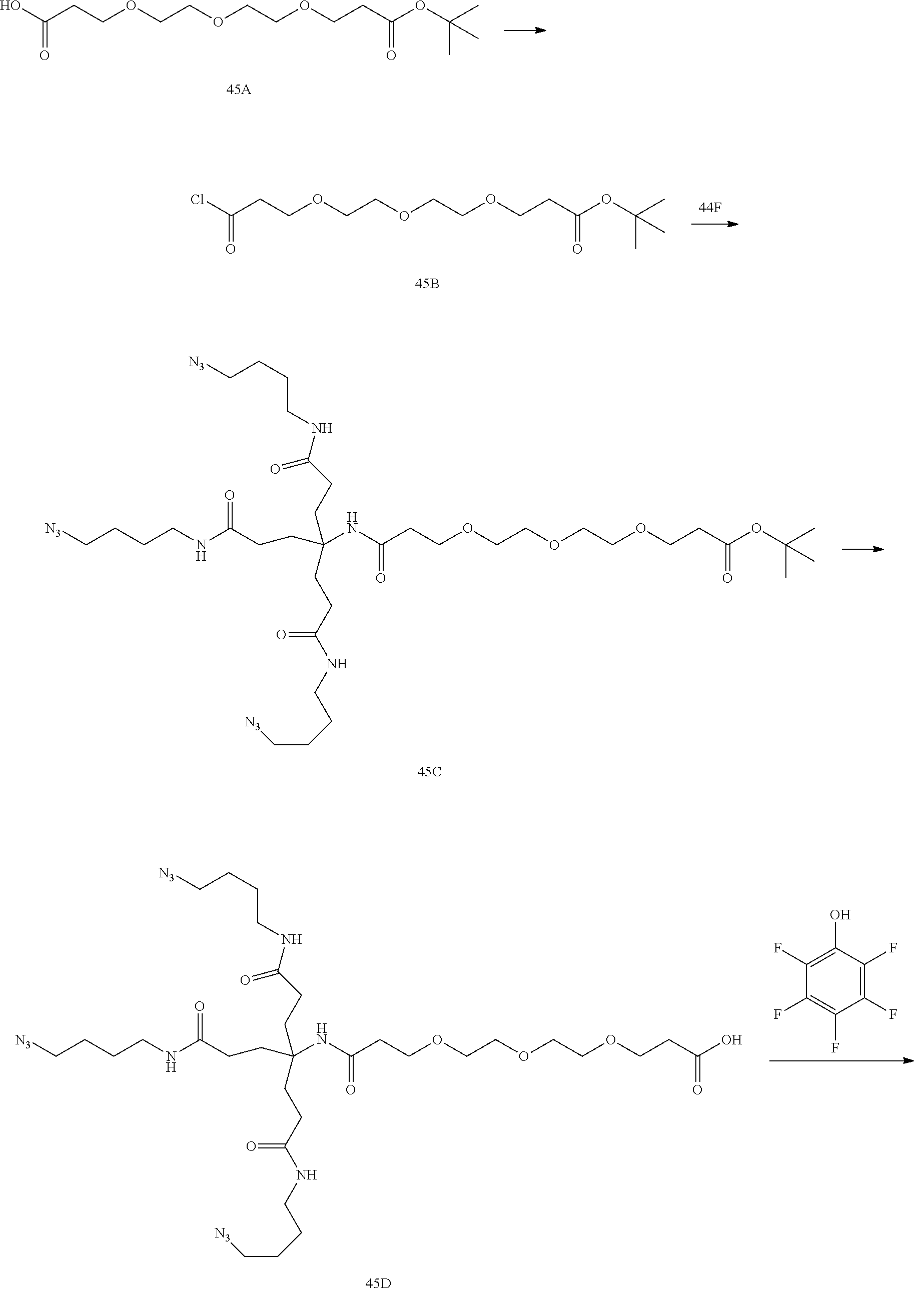 [SMILES: CC(C)(C)OC(=O)CCOCCOCCOCCC(=O)O ; CC(C)(C)OC(=O)CCOCCOCCOCCC(=O)Cl ; CC(C)(C)OC(=O)CCOCCOCCOCCC(=O)NC(CCC(=O)NCCCCN=[N+]=[N-])(CCC(=O)NCCCCN=[N+]=[N-])CCC(=O)NCCCCN=[N+]=[N-] ; [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)O ; Oc1c(F)c(F)c(F)c(F)c1F]

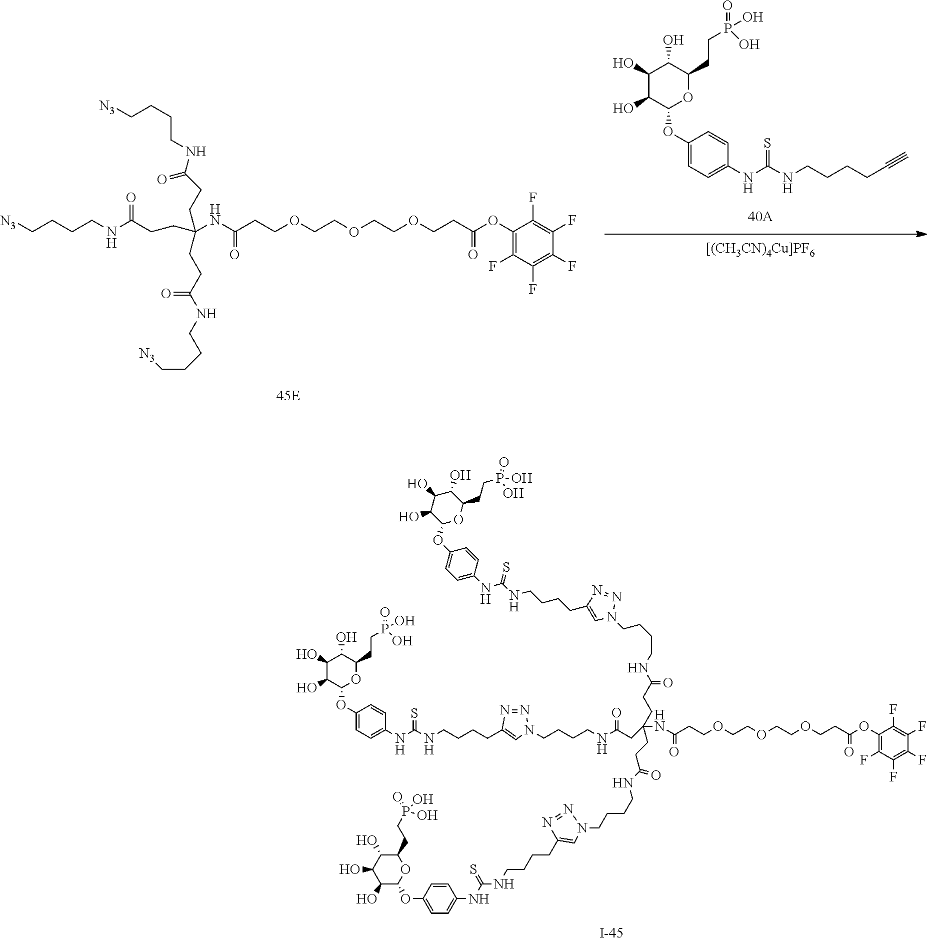

Compound I-45 was prepared using similar methods. Yield: 54.1 mg, 58%; H NMR (300 MHz, DMSO-d₆ with D₂O) δ 7.83 (s, 3H), 7.13 (d, J=8.5 Hz, 6H), 7.00 (d, J=8.6 Hz, 6H), 5.34 (s, 3H), 4.26 (bs, 6H), 3.88-2.87 (m, 40H), 2.64-2.53 (m, 6H), 2.04-1.40 (m, 40H), 1.36-1.11 (m, 8H).

### Example 46: Synthesis of Compound I-46

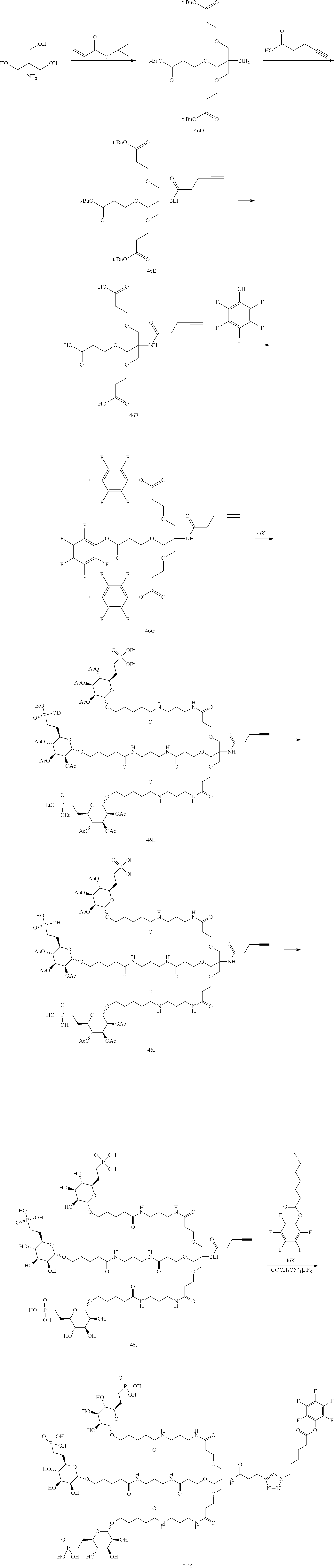

Compound I-46 was prepared using similar methods. Yield: 0.0035 g, 11.6%; LCMS m/z 965.68 [M+2]++; ¹H NMR (400 MHz, DMSO-d₆) δ 7.87 (t, J=4.8 Hz, 3H), 7.82-7.78 (m, 4H), 7.20-7.17 (m, 1H), 4.54 (s, 3H), 4.30 (t, J=7.2 Hz, 3H), 3.70 (bs, 1H), 3.56-3.52 (m, 6H), 3.39-3.25 (m, 25H), 3.22 (d, J=6.4 Hz, 8H), 3.02 (bs, 13H), 2.78 (t, J=7.6 Hz, 3H), 2.41 (t, J=8.0 Hz, 2H), 2.27 (t, J=6.0 Hz, 6H), 2.10-2.06 (m, 6H), 2.05-1.98 (m, 3H), 1.84-1.77 (m, 3H), 1.74-1.64 (m, 6H), 1.60-1.39 (m, 25H), 1.35-1.28 (m, 3H).

### Example 47: Synthesis of Compound I-47

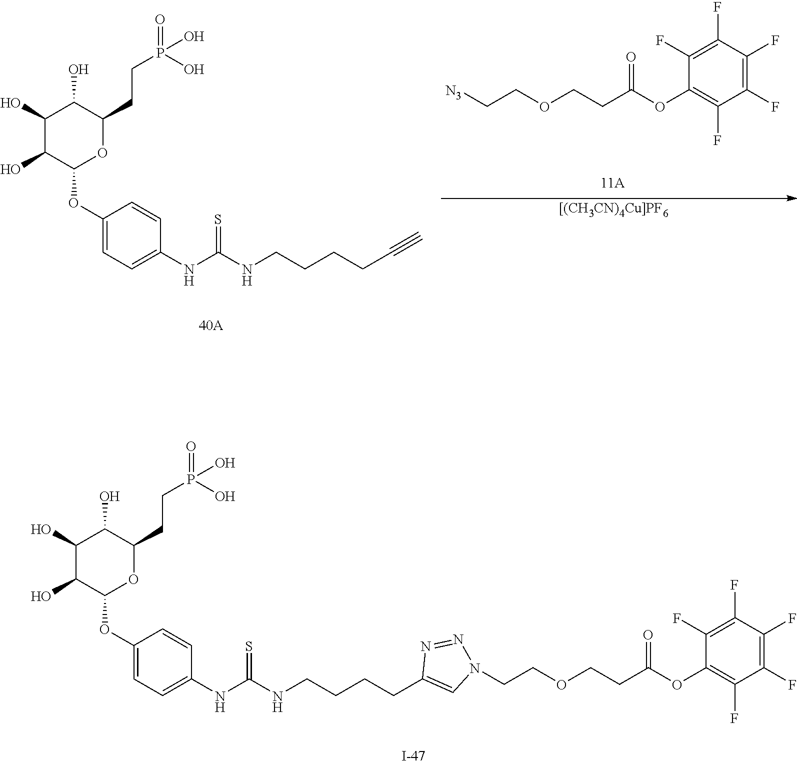

Compound I-47 was prepared using similar methods. Yield: 35.8 mg, 75%; LCMS m/z 814.4 [M+1]+; ¹H NMR (300 MHz, DMSO-d₆ with D₂O) δ 7.73 (s, 1H), 7.31-7.15 (m, 2H), 7.04-6.85 (m, 2H), 5.31 (s, 1H), 4.45 (t, J=5.2 Hz, 2H), 3.89-3.23 (m, 8H), 3.03-2.91 (m, 2H), 2.62-2.39 (m, 4H), 2.00-1.81 (m, 1H), 1.71-1.39 (m, 6H), 1.32-1.09 (m, 1H).

### Example 48: Synthesis of Compound I-48

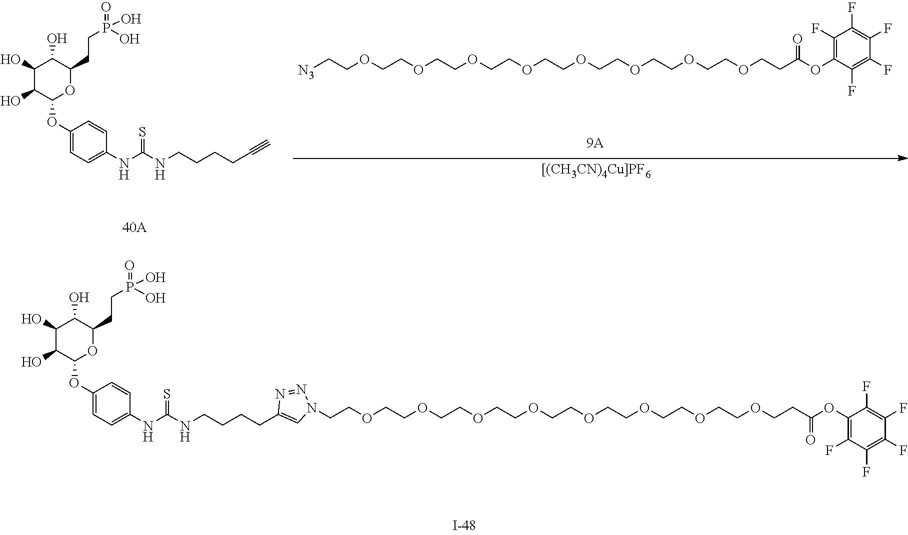

Compound I-48 was prepared using similar methods. Yield: 64.4 mg, 70%; LCMS m/z 1122.6 [M+1]+; ¹H NMR (300 MHz, DMSO-d₆ with D₂O) δ 7.80 (s, 1H), 7.25 (d, J=8.6 Hz, 2H), 6.97 (d, J=8.5 Hz, 2H), 5.32 (s, 1H), 4.44 (t, J=5.2 Hz, 2H), 3.85-3.20 (m, 36H), 2.99 (t, J=5.8 Hz, 2H), 2.67-2.37 (m, 4H), 2.02-1.82 (m, 1H), 1.70-1.38 (m, 6H), 1.30-1.05 (m, 1H).

### Example 49: Synthesis of Compound I-49

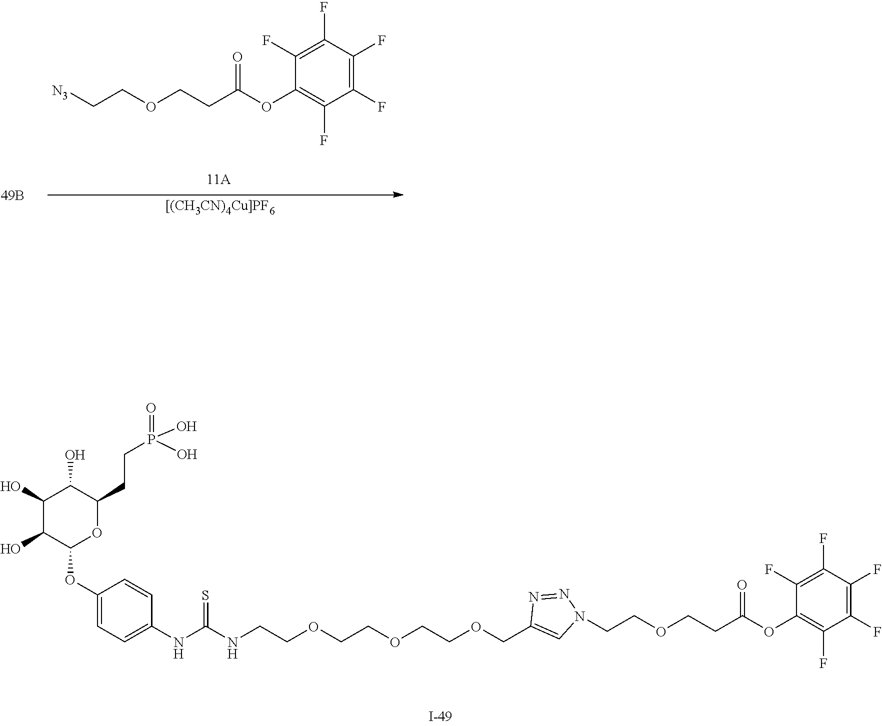

Compound I-49 is prepared from 49B using similar methods. Yield: 44.2 mg, 66%; LCMS m/z 904.4 [M+1]+; ¹H NMR (300 MHz, DMSO-d₆ with D₂O) δ 7.96 (s, 1H), 7.27 (d, J=8.5 Hz, 2H), 6.98 (d, J=8.7 Hz, 2H), 5.32 (s, 1H), 4.57-4.40 (m, 4H), 3.89-3.22 (m, 18H), 2.98 (t, J=5.8 Hz, 2H), 2.59-2.35 (m, 2H), 2.02-1.82 (m, 1H), 1.73-1.41 (m, 2H), 1.34-1.11 (m, 1H).

### Example 50: Synthesis of Compound I-50

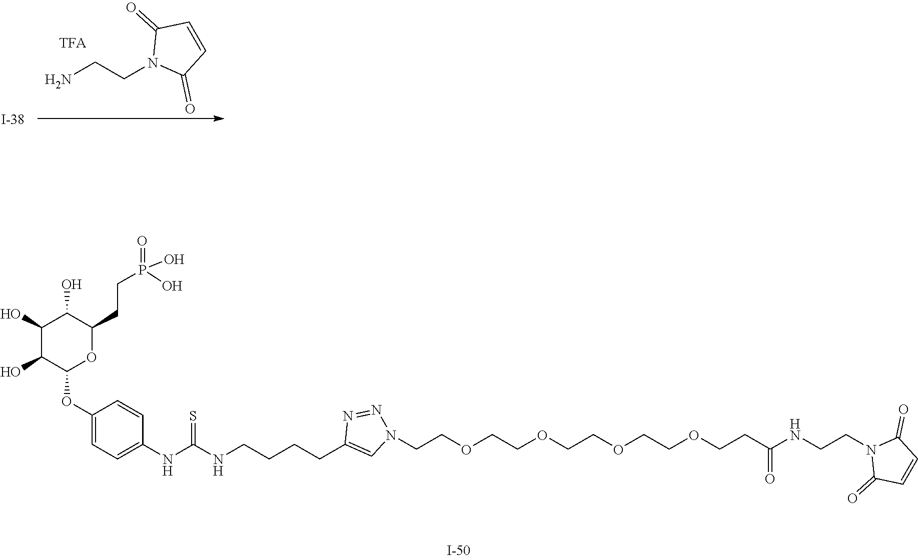

Compound I-50 is prepared from I-38 using similar methods. Yield: 25.8 mg, 72%; LCMS m/z 886.6 [M+1]+; ¹H NMR (300 MHz, DMSO-d₆ with D₂O) δ 7.78 (s, 1H), 7.29-7.18 (m, 2H), 6.95-6.81 (m, 4H), 5.24 (s, 1H), 4.42 (t, J=5.1 Hz, 2H), 3.97-3.25 (m, 22H), 3.21-3.11 (m, 2H), 3.05 (t, J=6.6 Hz, 2H), 2.66-2.54 (m, 2H), 2.25-2.14 (m, 2H), 2.03-1.81 (m, 1H), 1.69-1.34 (m, 6H), 1.31-1.07 (m, 1H).

### Example 51: Synthesis of Compound I-51

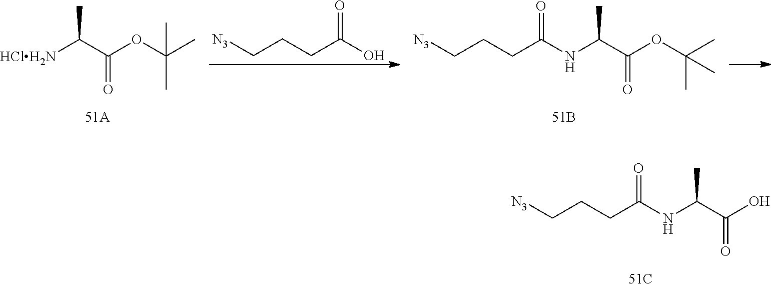

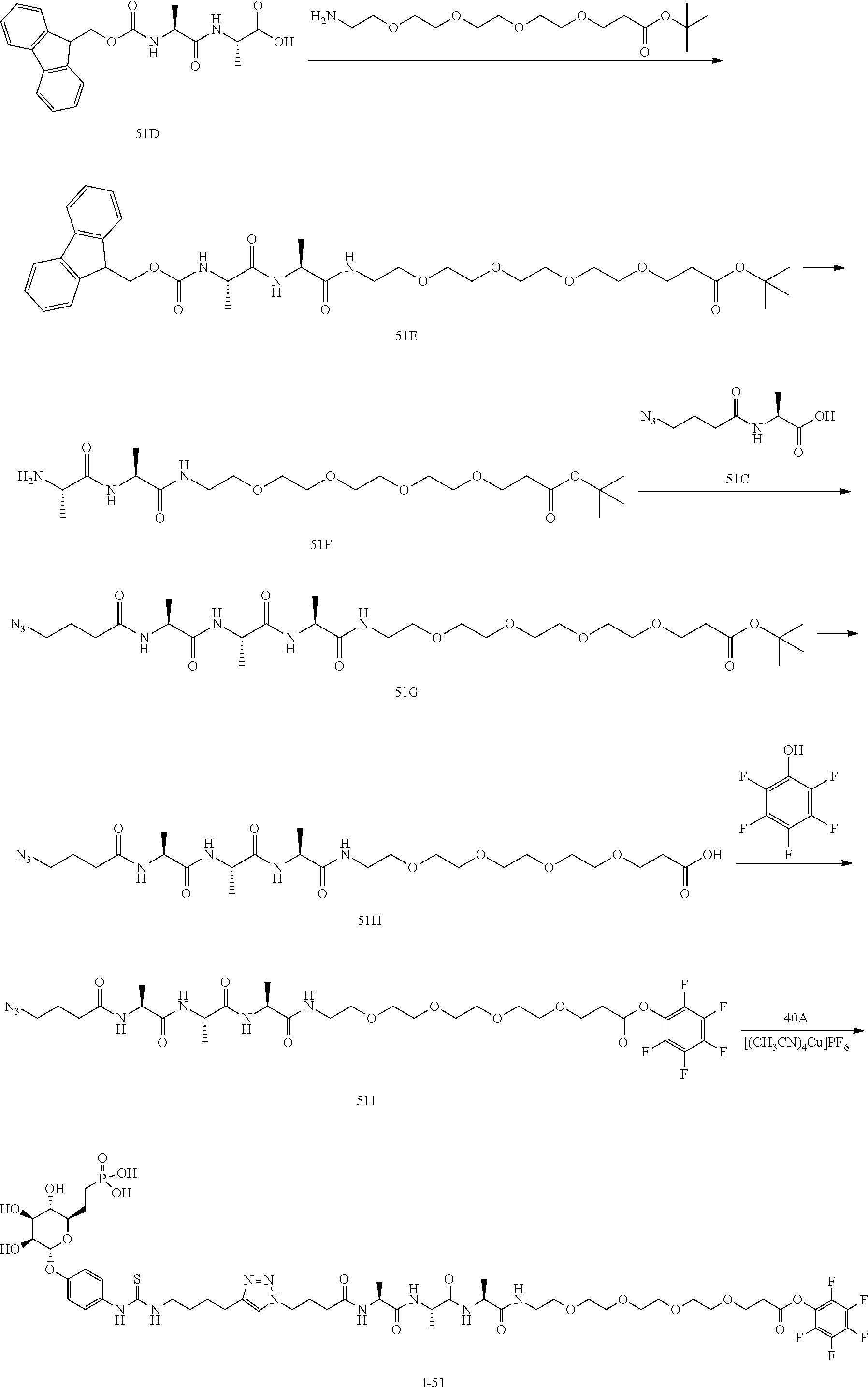

Compound I-51 was prepared according to the scheme above. Yield: 25.4 mg, 70%; LCMS m/z 1244.7 [M+1]+; H NMR (300 MHz, DMSO-d₆ with D₂O) δ 7.82 (s, 1H), 7.24 (d, J=8.2 Hz, 2H), 6.98 (d, J=8.7 Hz, 2H), 5.32 (s, 1H), 4.28 (t, J=6.9 Hz, 2H), 4.22-4.07 (m, 3H), 3.88-2.89 (m, 26H), 2.67-2.38 (m, 2H), 2.15-2.04 (m, 2H), 2.04-1.83 (m, 3H), 1.67-1.41 (m, 6H), 1.32-1.03 (m, 10H).

### Example 52: Synthesis of Compound I-52

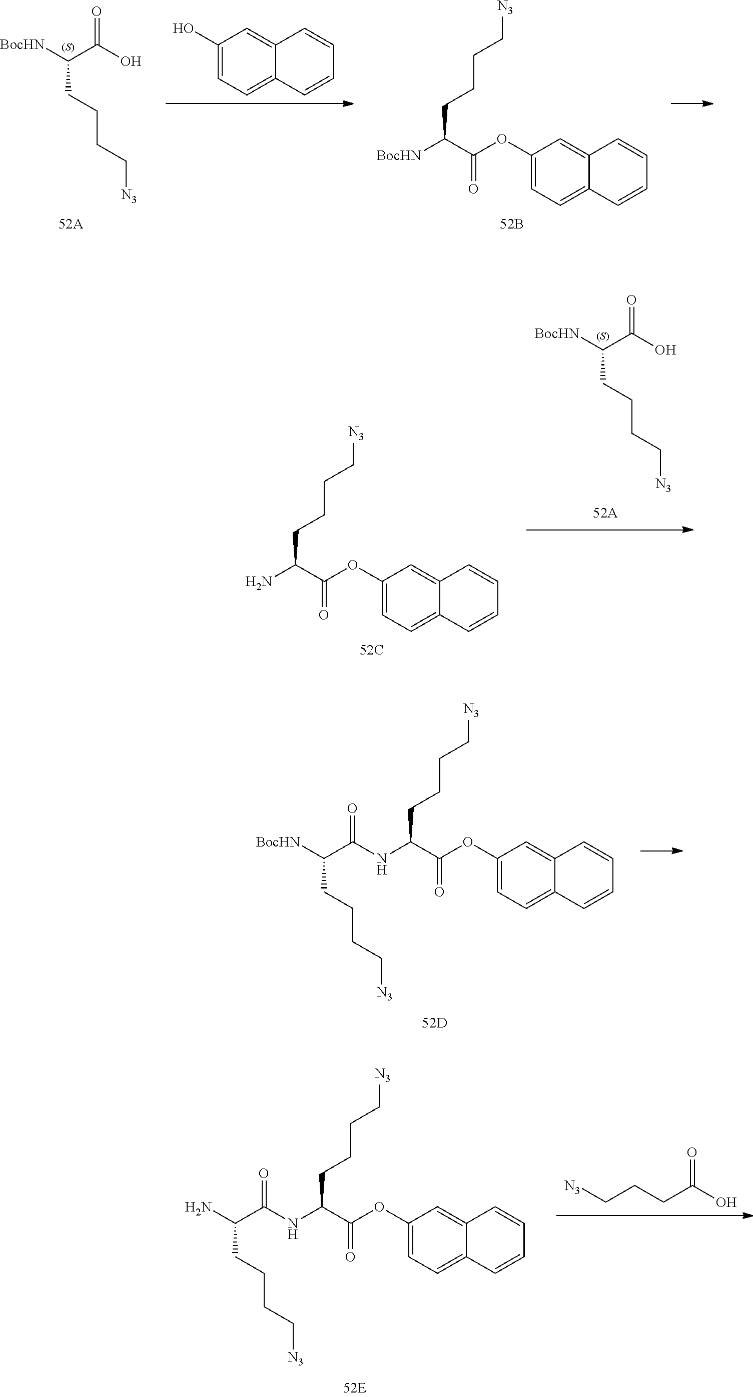

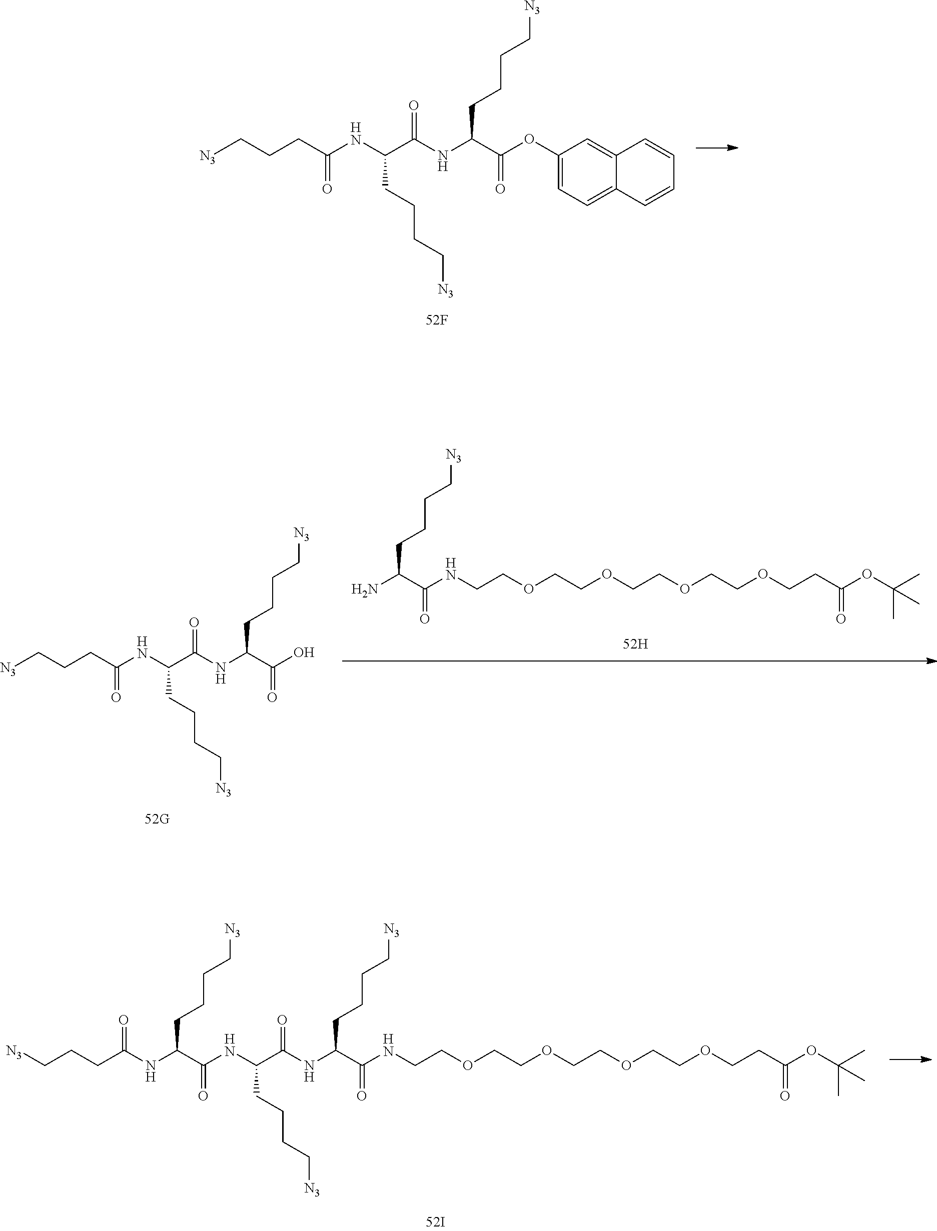 [SMILES: [N-]=[N+]=NCCCC[C@H](NC(=O)CCCN=[N+]=[N-])C(=O)N[C@@H](CCCCN=[N+]=[N-])C(=O)Oc1ccc2ccccc2c1 ; [N-]=[N+]=NCCCC[C@H](NC(=O)[C@H](CCCCN=[N+]=[N-])NC(=O)CCCN=[N+]=[N-])C(=O)O ; CC(C)(C)OC(=O)CCOCCOCCOCCOCCNC(=O)[C@@H](N)CCCCN=[N+]=[N-] ; CC(C)(C)OC(=O)CCOCCOCCOCCOCCNC(=O)[C@H](CCCCN=[N+]=[N-])NC(=O)[C@H](CCCCN=[N+]=[N-])NC(=O)[C@H](CCCCN=[N+]=[N-])NC(=O)CCCN=[N+]=[N-]]

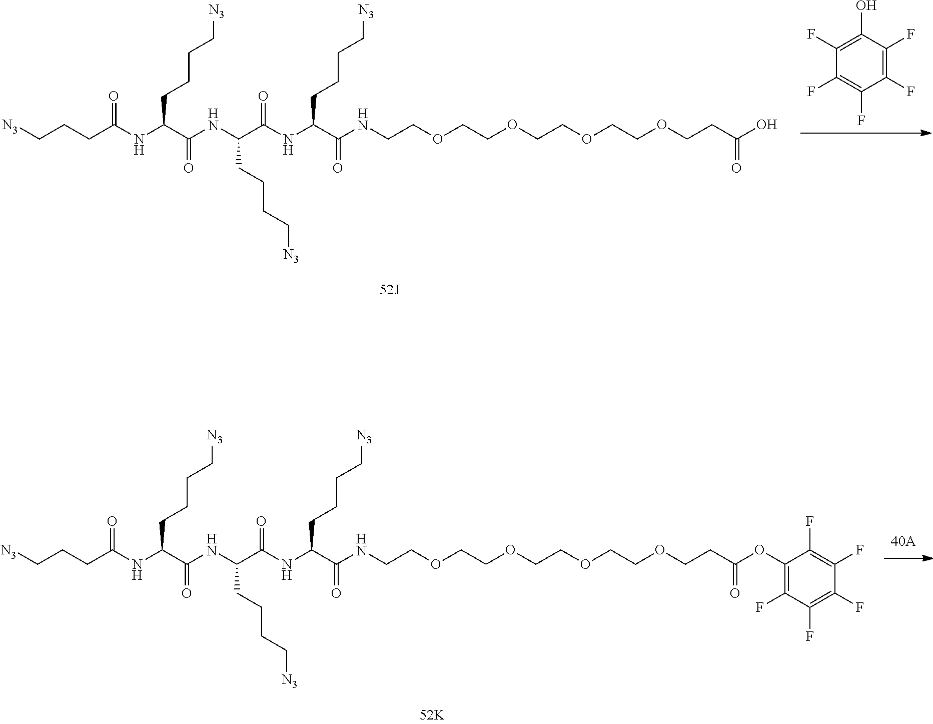 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)O ; Oc1c(F)c(F)c(F)c(F)c1F ; [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F]

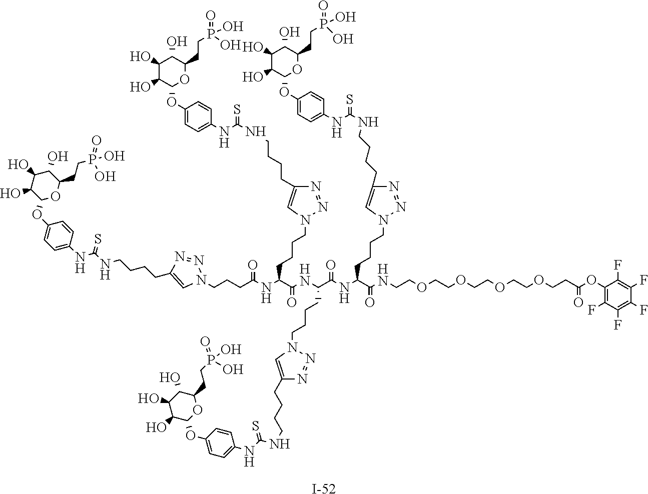

Compound 1-52 was prepared using similar methods. Yield: 14.4 mg, 70%; ¹H NMR (300 MHz, DMSO-d₆ with D₂O) δ 7.87-7.73 (m, 4H), 7.21-7.05 (m, 8H), 7.05-6.91 (m, 8H), 5.34 (s, 4H), 4.36-4.05 (in, 11H), 3.95-2.90 (m, 44H), 2.68-2.51 (m, 10H), 2.18-1.37 (m, 42H), 1.33-1.04 (in, 10H).

### Example 53: Synthesis of Compound I-53

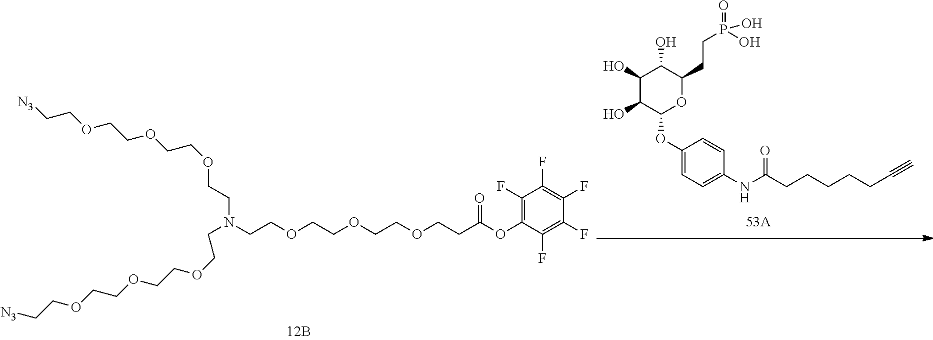 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F ; C#CCCCCCC(=O)Nc1ccc(O[C@H]2O[C@H](CCP(=O)(O)O)[C@@H](O)[C@H](O)[C@@H]2O)cc1]

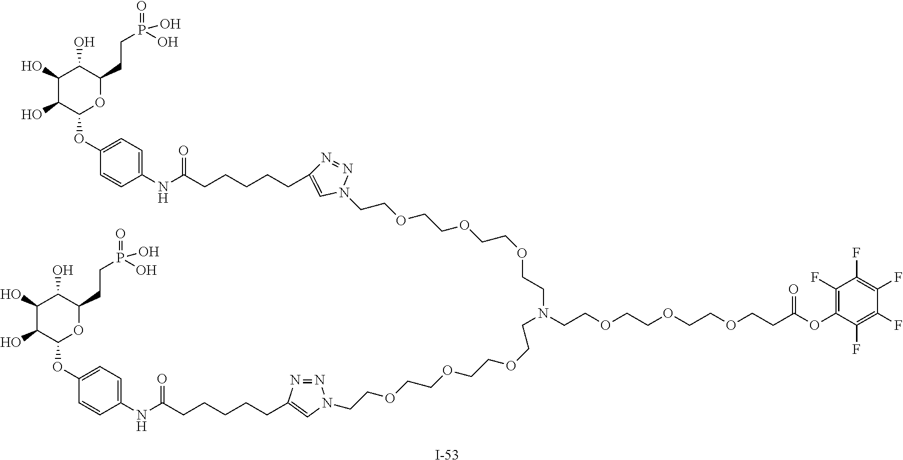

Compound I-53 is synthesized employing the procedures described for Compound I-52 using Compound 12B and (2-((2R,3S,4S,5S,6R)-3,4,5-trihydroxy-6-(4-(oct-7-ynamido)phenoxy)tetrahydro-2H-pyran-2-yl)ethyl)phosphonic acid (39B/53A) in lieu of Compound 52K and Synthon 40A.

### Example 54: Synthesis of Compound I-54

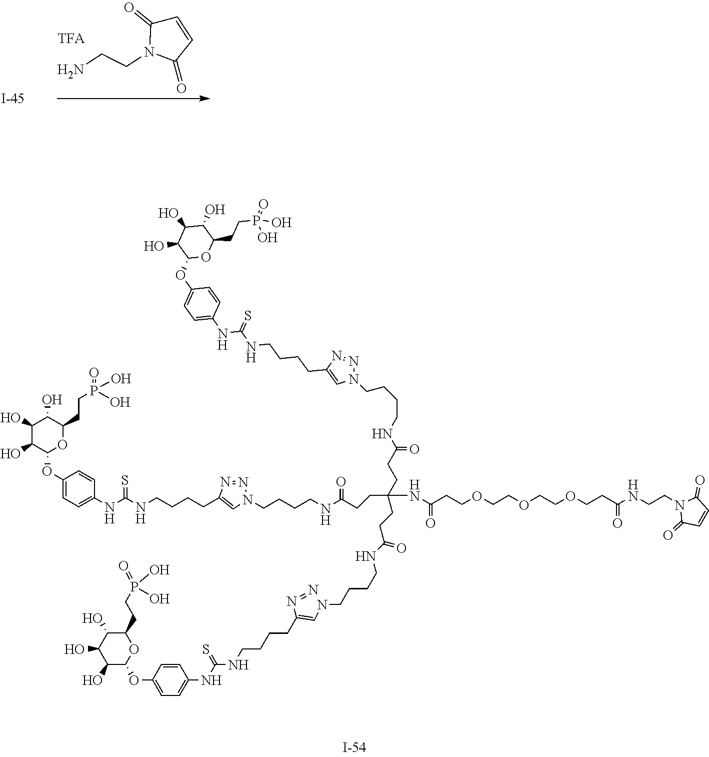

Compound I-54 is synthesized employing the procedures described for Compound I-50 using Compound I-45 in lieu of Compound I-38.

### Example 55: Synthesis of Compound I-55

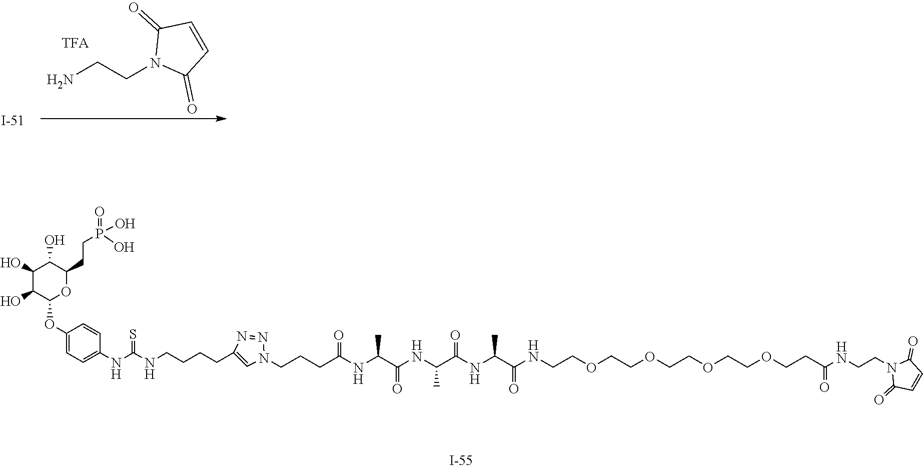

Compound I-55 is synthesized employing the procedures described for Compound I-50 using Compound I-52 in lieu of Compound I-38.

### Example 56: Synthesis of Compound I-56

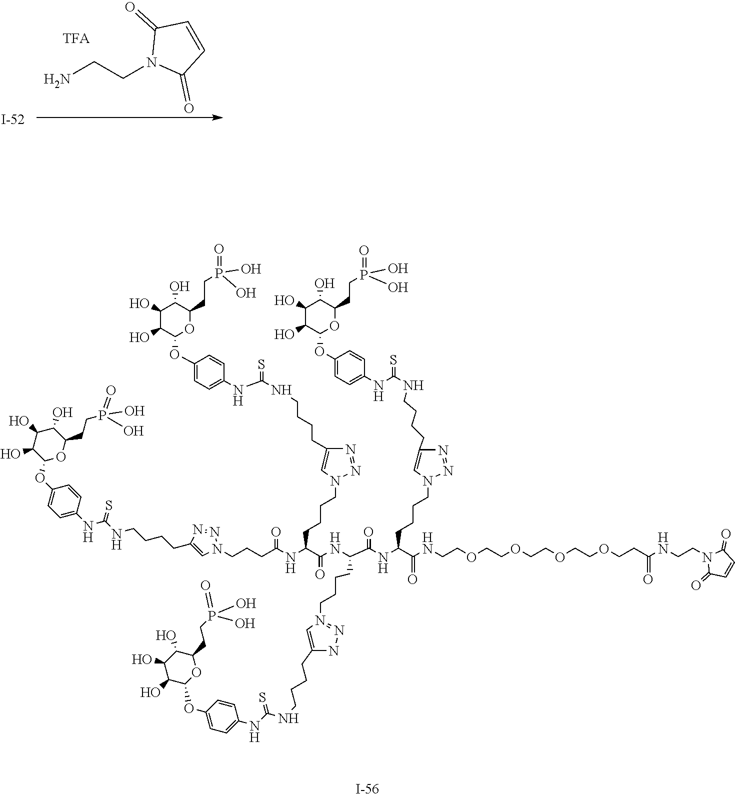

Compound I-56 is synthesized employing the procedures described for Compound I-50 using Compound I-52 in lieu of Compound I-38.

### Example 57: Synthesis of Compound I-57

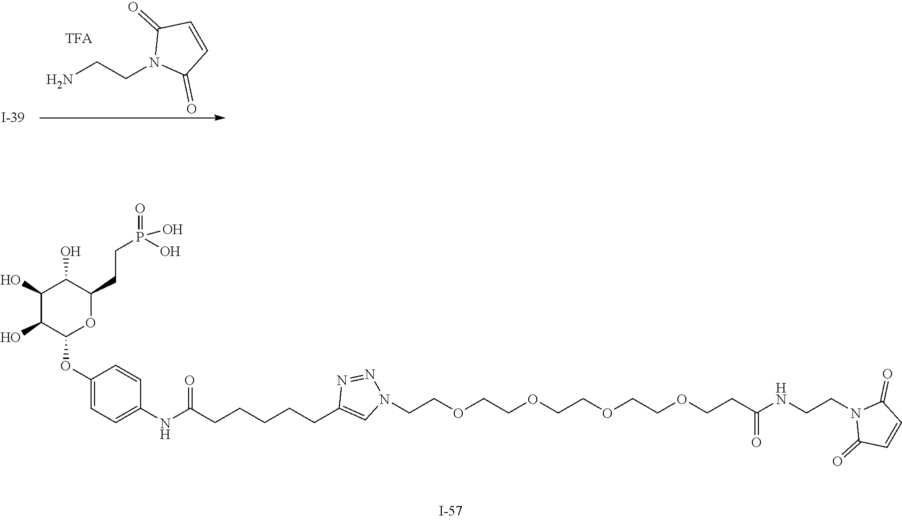

Compound I-57 is synthesized employing the procedures described for Compound I-50 using Compound I-39 in lieu of Compound I-38.

### Example 58: Synthesis of Compound I-58

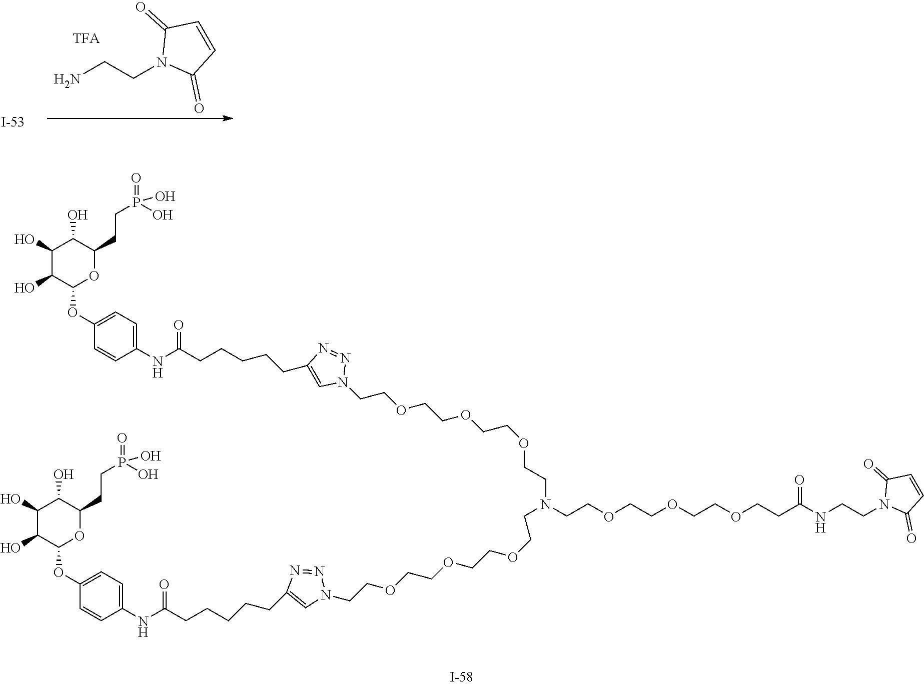

Compound I-58 was synthesized employing the procedures described for Compound I-50 using Compound I-53 in lieu of Compound I-38.

### Example 59: Synthesis of Compound I-59

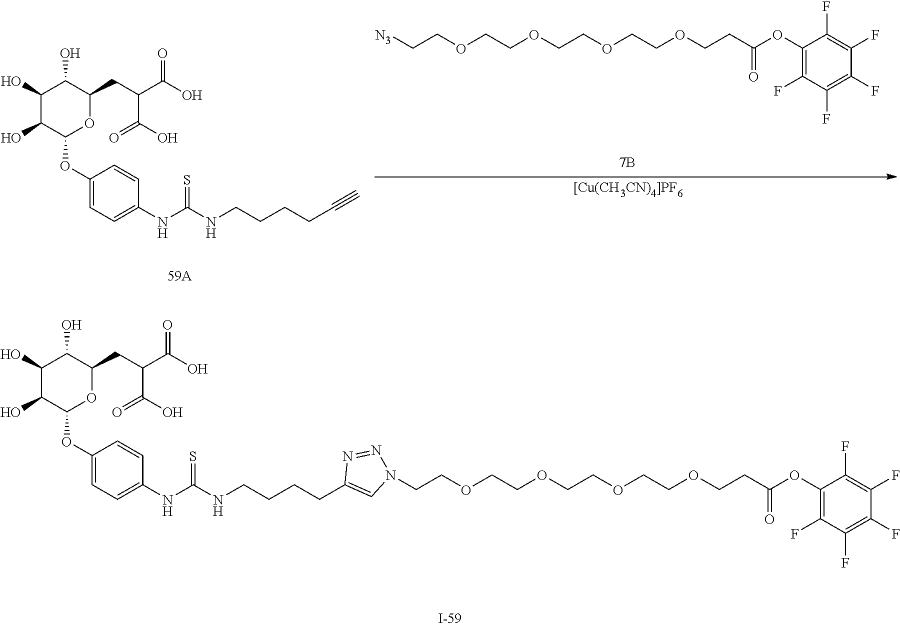

Compound I-59 (18 mg, 47% yield) was prepared using similar methods. LCMS m/z 954.5 [M+1]+; H NMR (300 MHz, DMSO-d₆) δ 7.76 (s, 1H), 7.16 (d, J=8.2 Hz, 2H), 6.94 (d, J=8.4 Hz, 2H), 5.27 (s, 1H), 4.41 (t, J=4.8 Hz, 2H), 3.84-2.81 (m, 25H), 2.65-2.20 (m, 3H), 1.75-1.41 (m, 5H).

### Example 60: Synthesis of Compound I-60

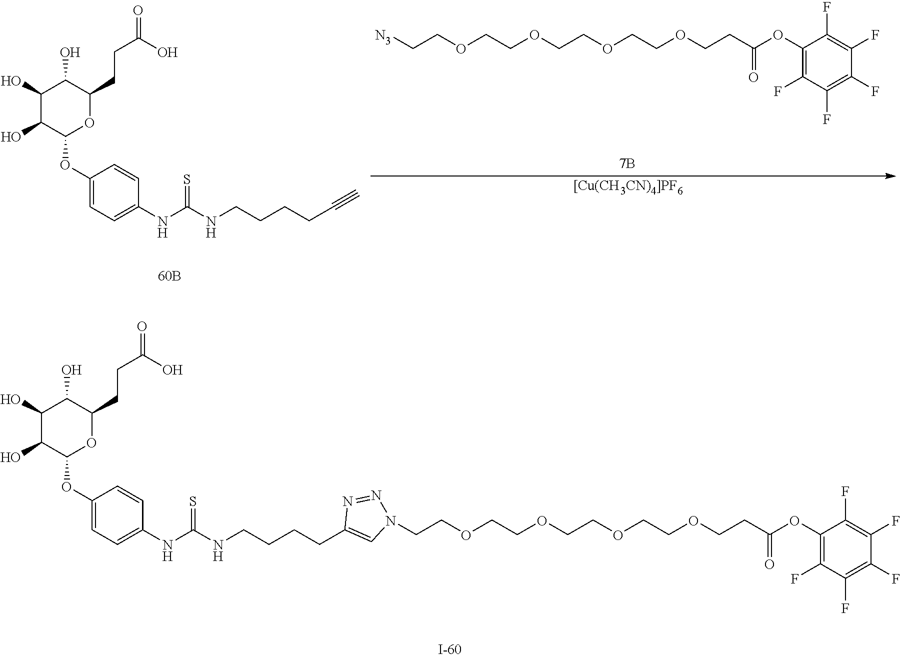

Compound I-60 (44 mg, 38 yield) was prepared from 60B using similar methods. LCMS m/z 910.6 [M+H]⁺.

### Example 61: Synthesis of Compound I-61

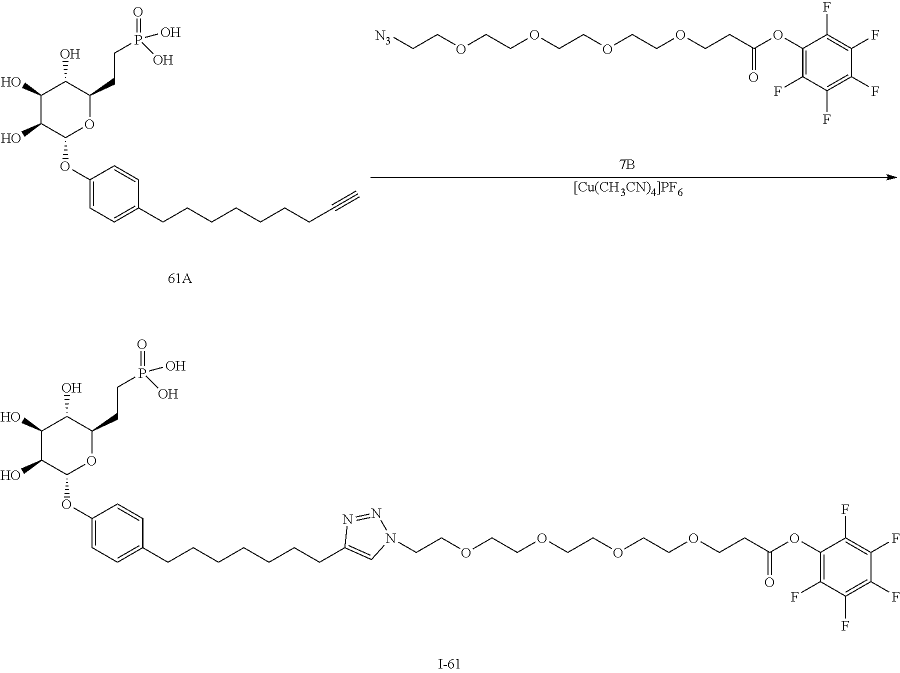

Compound I-61 is synthesized employing the procedures described for Compound I-60 using Compound 61A in lieu of Compound 60B. Yield: 33.8 mg, 55%; LCMS m/z 914.5 [M+1]+; ¹H NMR (300 MHz, DMSO-d₆ with D₂O) δ 7.74 (s, 1H), 7.06 (d, J=7.7 Hz, 2H), 6.89 (d, J=8.6 Hz, 2H), 5.27 (s, 1H), 4.40 (t, J=4.8 Hz, 2H), 3.82-3.67 (m, 5H), 3.61 (d, J=8.4 Hz, 1H), 3.54-3.24 (m, 14H), 2.93 (t, J=6.0 Hz, 2H), 2.59-2.37 (m, 4H), 1.95-1.79 (m, 1H), 1.63-1.38 (m, 6H), 1.31-1.06 (m, 7H).

### Example 62: Synthesis of Compound I-62

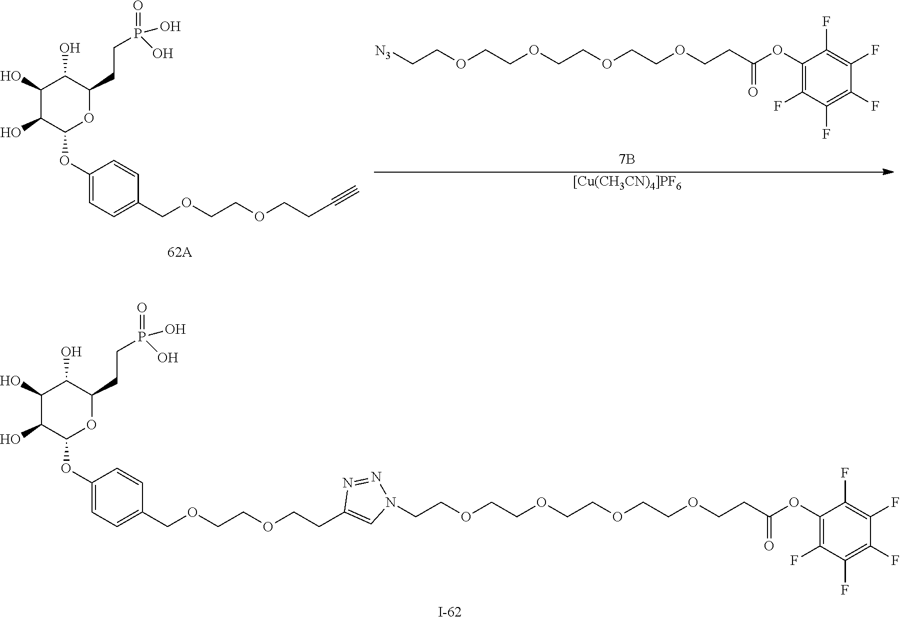

Compound I-62 is synthesized employing the procedures described for Compound I-60 using Compound 62A in lieu of Compound 60B.

### Example 63: Synthesis of Compound I-63

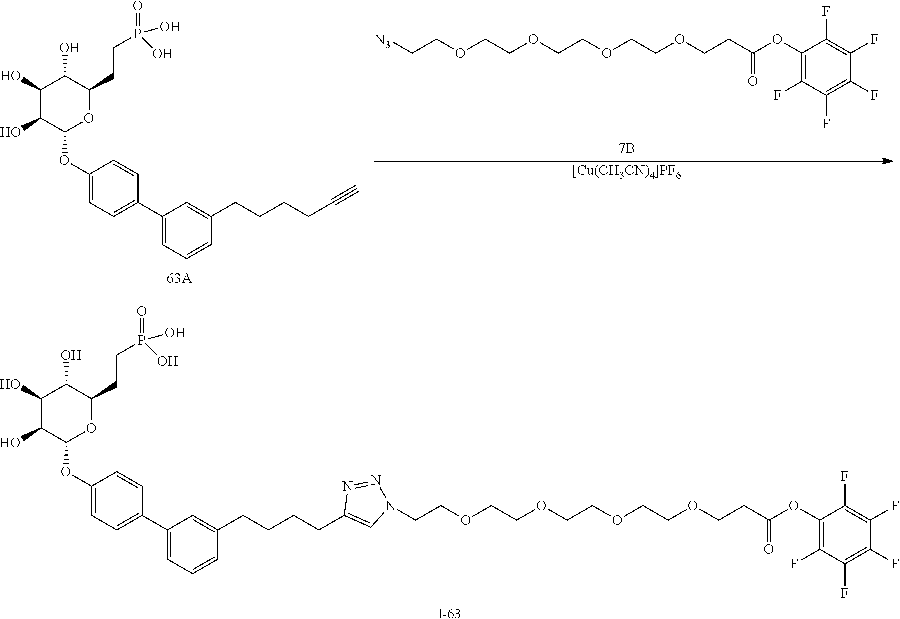

Compound I-63 is synthesized employing the procedures described for Compound I-60 using Compound 63A in lieu of Compound 60B.

### Example 64: Synthesis of Compound I-64

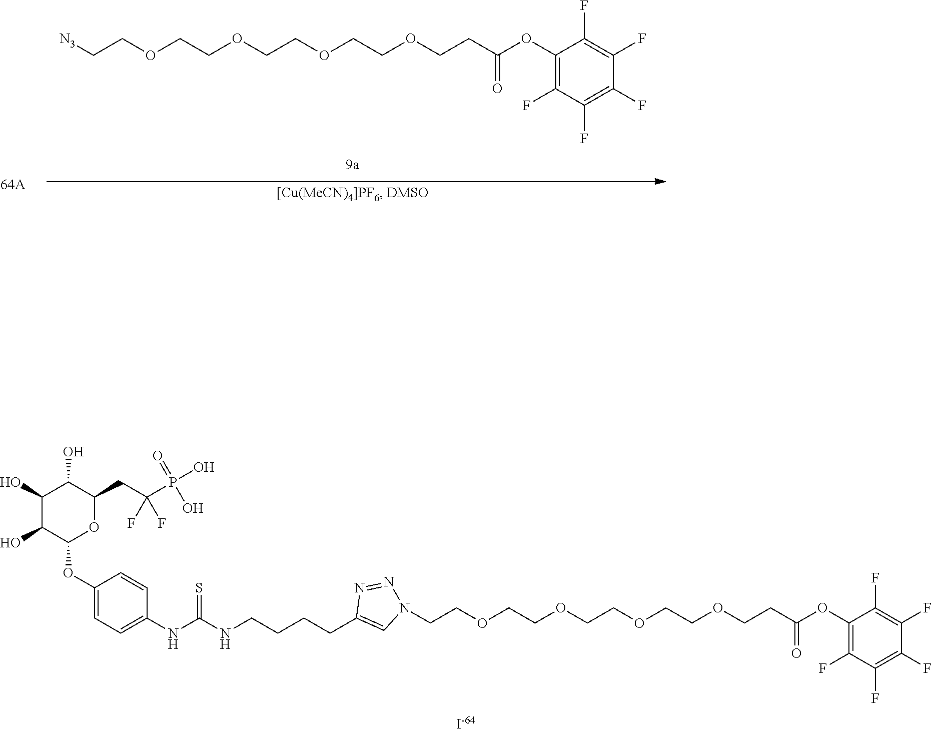

Compound I-64 was prepared from 64A using similar methods. Yield: 0.032 g, 33%; LCMS m/z 982.4 [M+1]+; ¹H NMR (400 MHz, DMSO-d6) δ 9.26 (s, 1H), 7.81 (s, 1H), 7.56 (s, 1H), 7.23 (d, J=8.8 Hz, 2H), 7.00 (d, J=8.8 Hz, 2H), 5.20 (s, 1H), 5.06 (s, 1H), 4.82 (s, 1H), 4.45 (t, J=10.0 Hz, 2H), 3.87-3.74 (m, 7H), 3.67 (t, J=9.6 Hz, 1H), 3.54-3.51 (m, 3H), 3.49-3.30 (m, 13H), 3.01 (t, J=5.6 Hz, 2H), 2.66-2.50 (m, 4H), 2.07-1.95 (m, 1H), 1.63-1.57 (m, 4H).

### Example 65: Synthesis of Compound I-65

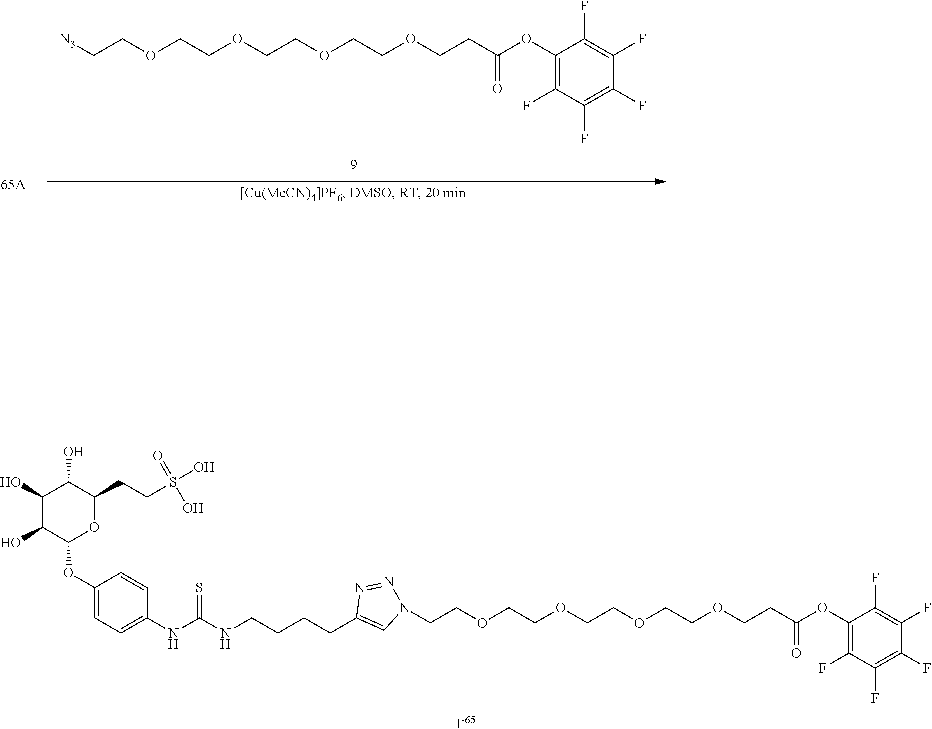

Compound I-65 was prepared from 65A using similar methods. HPLC (30-70% acetonitrile in water with 0.1% TFA) to obtain (I-65) as white solid. Yield: 20.0 mg, 33%; LCMS m/z 946.4 [M+1]+; ¹H NMR (400 MHz, D₂O) 7.90 (s, 1H), 7.20-7.18 (m, 4H), 5.63 (s, 1H), 4.60 (brs, 2H), 4.19 (s, 1H), 4.03-3.93 (m, 5H), 3.71-3.55 (m, 16H), 3.07 (brs, 2H), 2.92 (brs, 1H), 2.77 (s, 2H), 2.64-2.63 (1H), 2.25 (brs, 1H), 1.88 (brs, 1H), 1.74-1.59 (m, 4H).

### Example 66: Synthesis of Compound I-66

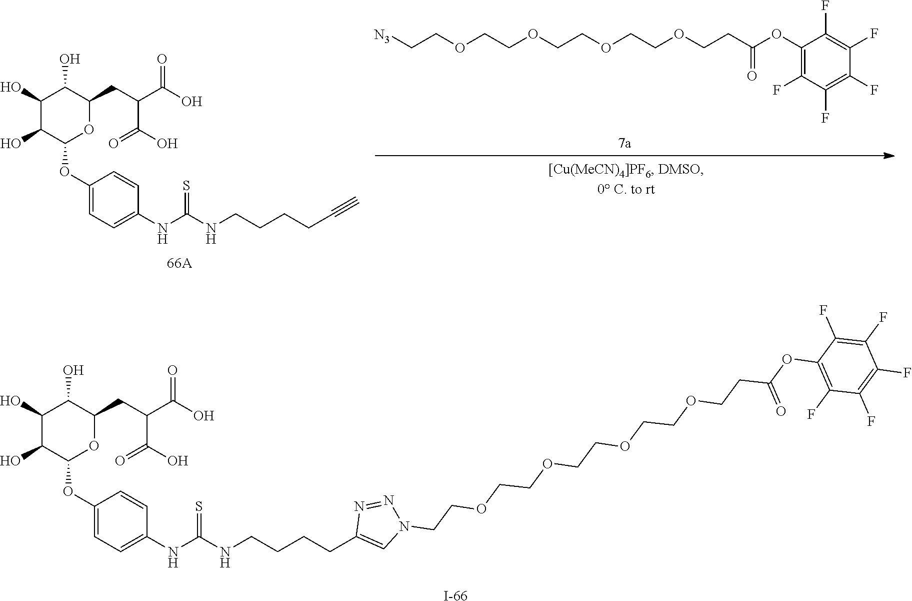

Compound I-66 was prepared from 66A using similar methods. Yield: 0.012 g, 31%; LC-MS m/z 954.3 [M+1]+; ¹H-NMR (400 MHz, DMSO-d6) δ 9.31 (bs, 1H), 7.81 (s, 1H), 7.44 (bs, 1H), 7.23-7.21 (m, 2H), 6.95 (d, J=8.8 Hz, 2H), 5.25 (s, 1H), 4.46-4.43 (m, 1H), 3.79-3.74 (m, 4H), 3.62-3.57 (m, 1H), 3.53-3.47 (m, 15H), 3.32 (bs, 5H), 3.23-3.19 (m, 1H), 3.03-3.00 (m, 2H), 2.66-2.60 (m, 2H), 2.36-2.32 (m, 1H), 1.71-1.56 (m, 6H).

### Example 67: Synthesis of Compound I-67 (See Above)

### Example 68: Synthesis of Compound I-68 (See Above)

### Example 69: Synthesis of Compound I-69

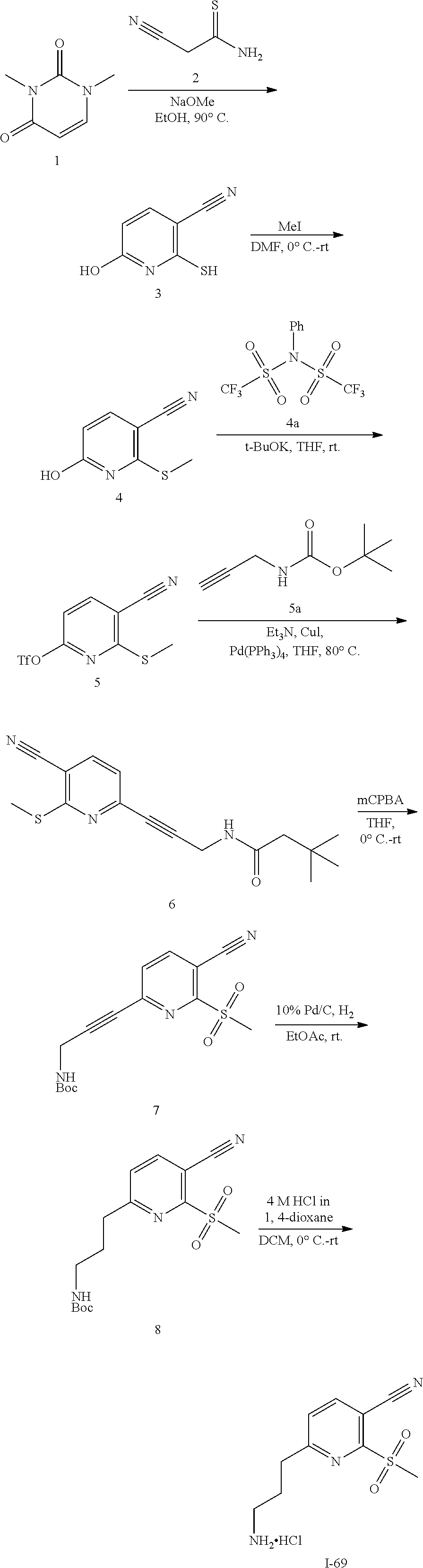

To a solution of 1,3-dimethylpyrimidine-2,4(1H,3H)-dione (1, 1.0 eq, 14.0 g, 99.9 mmol) in ethanol (150 mL), 25% sodium methoxide in methanol (2.0 eq, 44.0 mL, 200 mmol) and 2-cyanoethanethioamide (2, 1.0 eq, 10.0 g, 99.9 mmol) were added at room temperature, the resulting reaction mixture was stirred at 90° C. for 8 h. After completion, solvent was concentrated and residue was triturated with acetone, solid precipitated was filtered off and dried under vacuum to afford sodium 6-hydroxy-2-mercaptonicotinonitrile (3) as pale yellow solid. Yield: 13.0 g, 74.75%; LCMS m/z 151.2 [M−1]⁻.

To a solution of 6-hydroxy-2-mercaptonicotinonitrile (3, 1.0 eq, 13.0 g, 74.6 mmol) in N,N-dimethylformamide (130 mL), iodomethane (4.65 mL, 1.0 eq., 74.6 mmol) was added at 0° C., the reaction mixture was stirred at room temperature for 30 min. After completion reaction, the reaction mixture was diluted with water and extract with ethyl acetate. The organic layer was dried over sodium sulfate, filtered, and concentrated under high vacuum to get crude. The crude was purified by flash column chromatography using 20-30% ethyl acetate in hexane to afford (4) as pale yellow solid. Yield: 4.0 g, 32.24%; LCMS m/z 167.1 [M+1]⁺.

To a solution of 1,1,1-trifluoro-N-phenyl-N-((trifluoromethyl)sulfonyl)methanesulfonamide (4a, 10.3 g, 1.2 eq., 28.9 mmol) in tetrahydrofuran (60.0 mL), potassium 2-methylpropan-2-olate (28.9 mL, 1.2 eq., 28.9 mmol) and 6-hydroxy-2-(methylthio)nicotinonitrile (4, 1.0 eq, 4.0 g, 24.1 mmol) were added at room temperature, the reaction mixture was stirred at room temperature for 16 h. After completion, the reaction mixture was diluted with water and extracted with ethyl acetate. The organic layer was dried over sodium sulfate, filtered, and concentrated under high vacuum to get crude. The crude was purified by flash Colum chromatography using 20-30% ethyl acetate in hexane to afford (5) as off white solid. Yield: 5.80 g, 80.8%; LCMS m/z 299.3 [M+1]⁺.

To a solution of (5, 1.0 eq, 5.80 g, 19.4 mmol) in tetrahydrofuran (40.0 mL), tert-butyl prop-2-yn-1-ylcarbamate (5a, 3.32 g, 1.1 eq., 21.4 mmol) and triethylamine (8.43 mL, 3 eq., 58.3 mmol) were at room temperature, the reaction mixture was degassed under nitrogen atmosphere. Palladium (2+) bis(triphenylphosphane) dichloride (0.682 g, 0.05 eq., 0.972 mmol) and copper(I) iodide (0.37 g, 0.1 eq., 1.94 mmol) were added. The reaction mixture was stirred at 80° C. for 3 h. After completion, the reaction mixture was diluted with water and extract with ethyl acetate, the organic layer was dried over sodium sulfate, filtered, and concentrated under high vacuum to get crude. The crude was purified by flash Colum chromatography using 20-30% ethyl acetate in hexane to afford (6) as pale yellow solid. Yield: 3.50 g, 59.32%; LCMS m/z 304.2 [M+1]⁺.

To a solution of (6, 1.0 eq, 3.30 g, 10.9 mmol) in tetrahydrofuran (30 mL), 3-chlorobenzene-1-carboperoxoic acid (8.64 g, 3 eq., 32.6 mmol) was added at 0° C., the reaction mixture was stirred at room temperature for 2 h. After completion, the reaction mixture was diluted with sodium bicarbonate solution and exacted with ethyl acetate. The organic layer was dried over sodium sulfate, filtered, and concentrated under high vacuum to get crude. The crude was purified by flash Colum chromatography using 30-50% ethyl acetate in hexane to afford (7) as pale yellow oil. Yield: 2.0 g, 42.21%; LCMS m/z 336.4 [M+1]⁺.

To a solution of (7, 1.0 eq, 2.0 g, 5.96 mmol) in ethyl acetate (30.0 mL), 10% Palladium on carbon (1.0 g) was added at room temperature, the reaction mixture was stirred at room temperature under hydrogen atmosphere for 3 h. After completion, the reaction mixture was filtered through celite bed, filtrate was concentrated and dried under vacuum to afford tert-butyl (3-(5-cyano-6-(methylsulfonyl)pyridin-2-yl)propyl)carbamate (8) as pale yellow viscous liquid. Yield: 1.00 g, 42.98%; LCMS m/z 336.4 [M+1]⁺.

To a solution of (8, 1.00 g, 2.95 mmol) in dichloromethane (10.0 mL), 4M HCl in 1,4-dioxane (6.00 mL) was added at 0° C. The resulting reaction mixture was stirred at room temperature for 4 h. After completion, solvent was concentrated and dried to get crude, the crude was washed with diethyl ether and n-pentane and dried to afford 6-(3-aminopropyl)-2-methanesulfonylpyridine-3-carbonitrile hydrochloride (I-69) as off white solid. Yield: 0.785 g, 96.62%; LC-MS m/z 240.07 [M+1]+; ¹H-NMR (400 MHz, DMSO-d6) δ 8.59 (d, J=8.0 Hz, 1H), 7.91 (bs, 3H), 7.85 (d, J=8.0 Hz, 1H), 3.56 (s, 1H), 3.47 (s, 3H), 3.04 (t, J=7.2 Hz, 1H), 2.87-2.82 (m, 2H), 2.06-1.99 (m, 2H).

### Example 70: Synthesis of Compound I-70 (See Above)

### Example 71: Synthesis of Compound I-71 (See Above)

### Example 72: Synthesis of Compound I-72

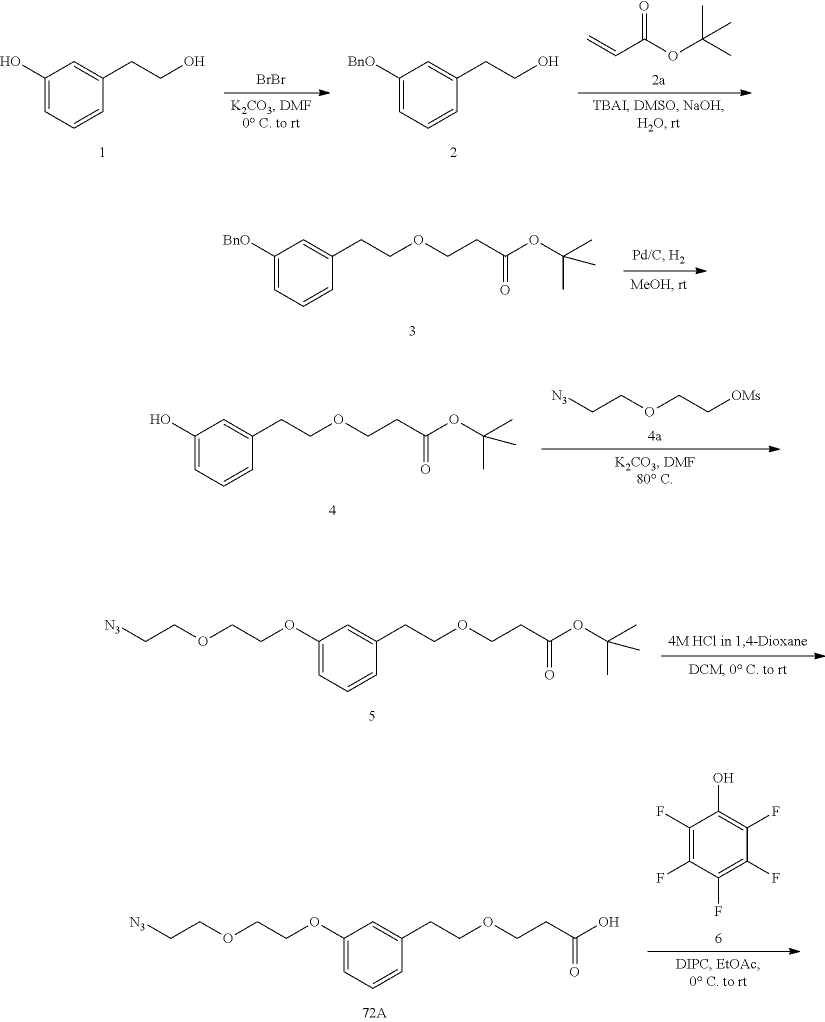

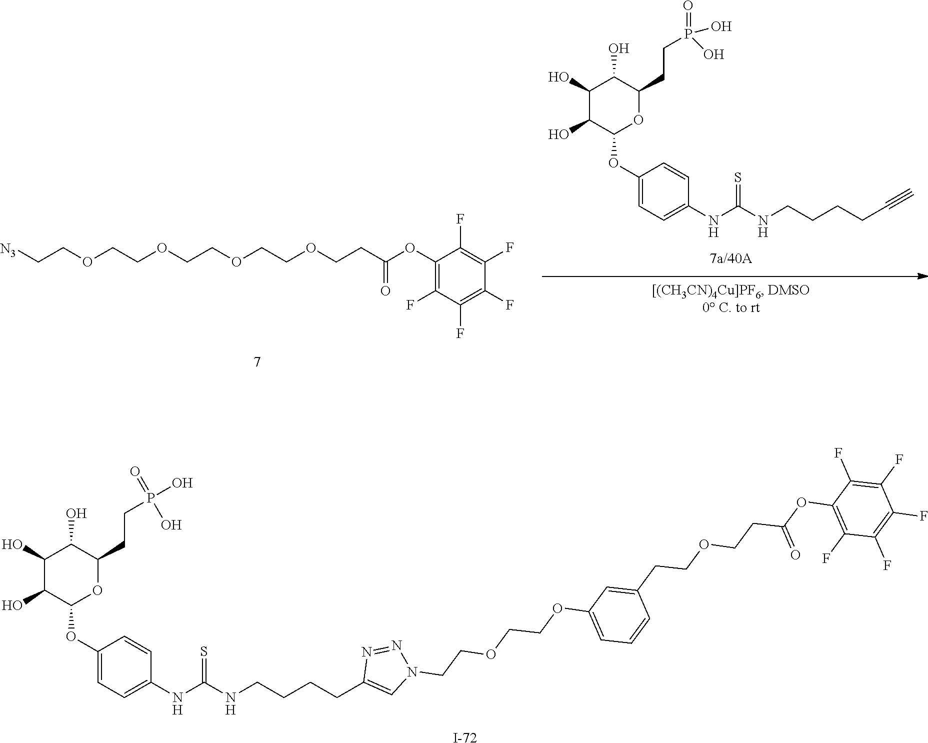

To a stirred solution of 3-(2-hydroxyethyl)phenol (1, 3.50 g, 1.0 eq, 25.3 mmol) in N,N-dimethylformamide (40 mL), potassium carbonate (7.00 g, 2 eq, 50.7 mmol) was added and reaction mixture cooled to 0° C. Benzyl bromide (6.02 mL, 2 eq, 50.7 mmol) was then added slowly and reaction mixture stirred at room temperature for 3 h. After completion, reaction mixture was diluted with water and extracted with ethyl acetate. Organic layer was dried over anhydrous sodium sulfate, filtered and concentrated under reduced pressure to get crude product which was purified by flash column chromatography using silica gel column and 20% Ethyl acetate in hexane as eluents to afford of 2-(3-(benzyloxy)phenyl)ethan-1-ol (2) as colorless sticky gum. Yield: 5.0 g, 86%; LC-MS m/z 229.20 [M+1]⁺.

To a stirred solution of (2, 5.00 g, 21.9 mmol) in Dimethylsufoxide (20.0 mL) at 0° C., sodium hydroxide (1.31 g, 1.5 eq, 32.9 mmol) dissolved in water (10.0 ml), tert-butyl prop-2-enoate (9.57 mL, 3 eq, 65.7 mmol), and tetrabutyl ammoniumiodide (1.62 g, 0.2 eq., 4.38 mmol) were added and reaction mixture stirred at room temperature for 4 h. After completion, reaction mixture was diluted with water and extracted with ethyl acetate. Ethyl acetate layer was dried over anhydrous sodium sulfate and concentrated under reduced pressure to get crude product which was purified by flash chromatography using silica gel column and 20% Ethyl acetate in hexanes as eluents. Desired fractions were concentrated under reduced pressure to afford (3) as colorless sticky gum. Yield: 7.0 g, 89%; LC-MS m/z 355.29 [M−1]⁻.

To a solution of (3, 7.00 g, 19.6 mmol) in methanol (50 mL) was added 10% palladium on carbon (0.80 g) and reaction mixture stirred under hydrogen atmosphere for 3 h. After completion reaction mixture filtered over celite pad and filtrate was concentrated under reduced pressure to afford (4) as colorless sticky gum. Yield:4.2 g, 80%; LC-MS m/z 267.25 [M+1]⁺

To a solution of (4, 0.700 g, 2.63 mmol) in N,N-dimethylformamide (5.00 mL) was added potassium carbonate (1.09 g, 3 eq, 7.88 mmol) and 2-(2-azidoethoxy)ethyl methanesulfonate (4a, 0.660 g, 1.2 eq, 3.15 mmol) and reaction mixture was heated at 80° C. for 17 h. TLC showed consumption of starting material. Reaction mixture cooled down and quenched by addition of water and extracted with ethyl acetate. Ethyl acetate layer was washed with water, brine solution dried over anhydrous sodium sulphate and concentrated under reduced pressure to get crude product. Crude product obtained was purified by flash chromatography using silica gel column and eluting product in 15 to 20% ethyl acetate in hexane as eluents. Desired fractions were concentrated under reduced pressure to afford tert-butyl (5) as colorless liquid. Yield: 0.50 g, 50%; LC-MS m/z 397.40 [M+18]⁺.

To a solution of (5, 0.400 g, 1.05 mmol) in dichloromethane (5.00 mL) at 0° C. was added 4N hydrochloric acid in 1,4-dioxane (5 mL) and reaction mixture was stirred at room temperature for 16 h, after completion reaction mixture was concentrated to get crude product which was purified by flash chromatography using silica gel column and 40% ethyl acetate in hexane as eluents. Desired fractions were concentrated under reduced pressure to afford 3-(3-(2-(2-azidoethoxy)ethoxy)phenethoxy)propanoic acid (72A) as colorless sticky gum. Yield: 0.183 g, 53%; LC-MS m/z 324.21 [M+18]+. ¹H-NMR (400 MHz, DMSO-d6) δ 12.15 (s, 1H), 7.19-7.16 (m, 1H), 6.80-6.75 (m, 3H), 4.07 (t, J=4.4 Hz, 2H), 3.78-3.75 (m, 2H), 3.66 (t, J=4.8 Hz, 2H), 3.61-3.54 (m, 4H), 3.43-3.40 (m, 2H), 2.75 (t, J=7.2 Hz, 2H), 2.43 (t, J=6.40 Hz, 2H).

To a solution of (72A, 0.200 g, 0.619 mmol) in ethyl acetate (2.0 mL) at 0° C. was added N,N′-Diisopropylcarbodiimide (0.097 mL, 0.619 mmol) and pentafluorophenol (6, 0.102 g, 0.9 eq, 0.557 mmol) and reaction mixture stirred at room temperature for 3 h. Reaction mixture filtered over celite bed and filtrate concentrated under reduced pressure to get crude product. Crude product obtained was purified by combiflash column chromatography using silica gel column and eluting compound in 0 to 10% Ethyl acetate in hexanes as eluents. Desired fractions were concentrated under reduced pressure to afford (7) as colorless sticky gum. Yield: 0.13 g, 43%; ¹H-NMR (400 MHz, CDCl₃) δ 7.22-7.17 (m, 1H), 6.82-6.76 (m, 3H), 4.13-4.08 (m, 2H), 3.87-3.79 (m, 4H), 3.76-3.73 (m, 2H), 3.69-362 (m, 2H), 3.43-3.40 (m, 2H), 2.93-2.84 (m, 4H).

To a solution of (7a, 0.043 g, 0.088 mmol) in dimethylsulfoxide (1.0 mL) was added perfluorophenyl 3-(3-(2-(2-azidoethoxy)ethoxy)phenethoxy)propanoate (7, 0.043 g, 1.0 eq, 0.088 mmol) in dimethylsulfoxide (0.5 mL) and reaction mixture cooled to 0° C. Tetrakis(acetonitrile)copper(I) hexafluorophosphate (0.082 g, 2.5 eq., 0.220 mmol) was added to reaction mixture and reaction mixture stirred at room temperature for 15 minutes. After completion reaction mixture was purified by reverse phase preparative HPLC using 30-70% acetonitrile in water with 0.1% TFA. Desired fractions were lyophilized to afford (I-72) as off white solid. Yield: 0.021 g, 24%; LC-MS m/z 978.36 [M+1]+. ¹H-NMR (400 MHz, DMSO-d6) δ 9.28 (s, 1H), 7.81 (s, 1H), 7.57 (bs, 1H), 7.25 (d, J=8.40 Hz, 2H), 7.15 (t, J=8.0 Hz, 1H), 6.98 (d, J=8.80 Hz, 2H), 6.80-6.78 (m, 2H), 6.73 (d, J=9.20 Hz, 2H), 5.32 (s, 1H), 4.48 (t, J=5.20 Hz, 2H), 4.01 (t, J=4.00 Hz, 2H), 3.84 (t, J=5.20 Hz, 2H), 3.80 (bs, 1H), 3.77-3.70 (m, 4H), 3.64-3.56 (m, 3H), 3.44 (bs, 2H), 3.36-3.28 (m, 2H), 3.01 (t, J=5.60 Hz, 2H), 2.77 (t, J=7.20 Hz, 2H), 2.59 (t, J=6.80 Hz, 2H), 1.96-1.92 (m, 1H), 1.55 (bs, 6H), 1.26-1.15 (m, 1H).

### Example 73-80: Synthesis of Compounds I-73 to I-80(See Above)

### Example 81: Synthesis of Compound I-81

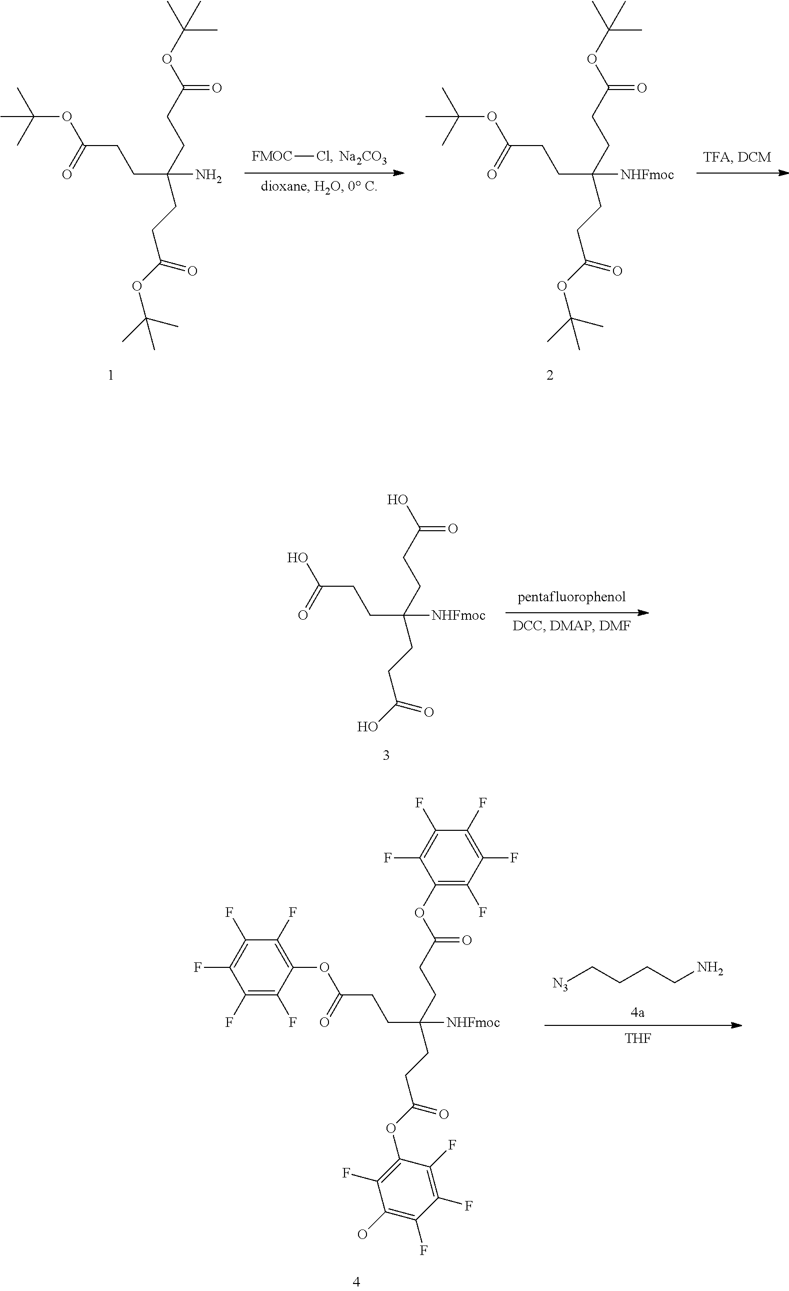

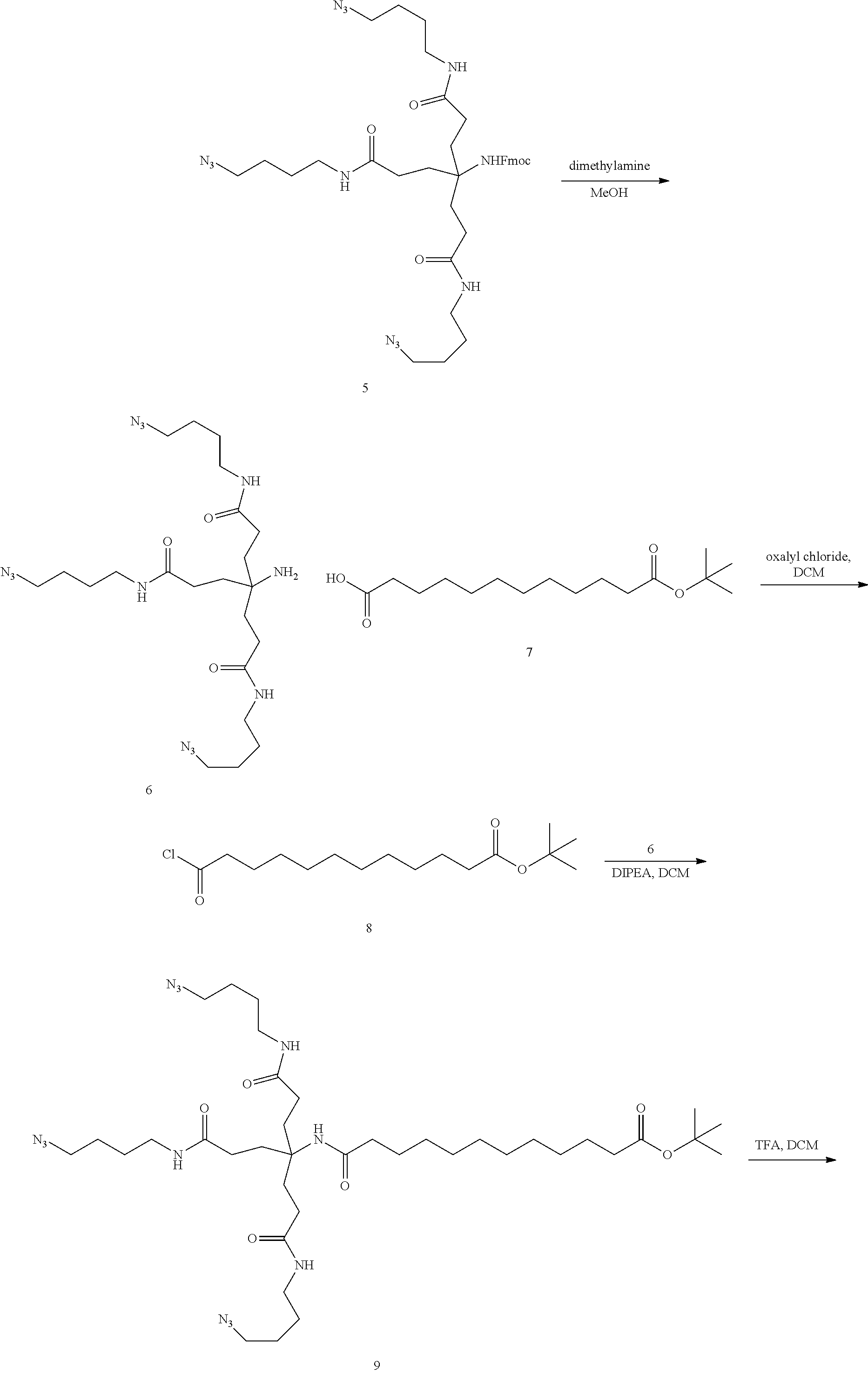

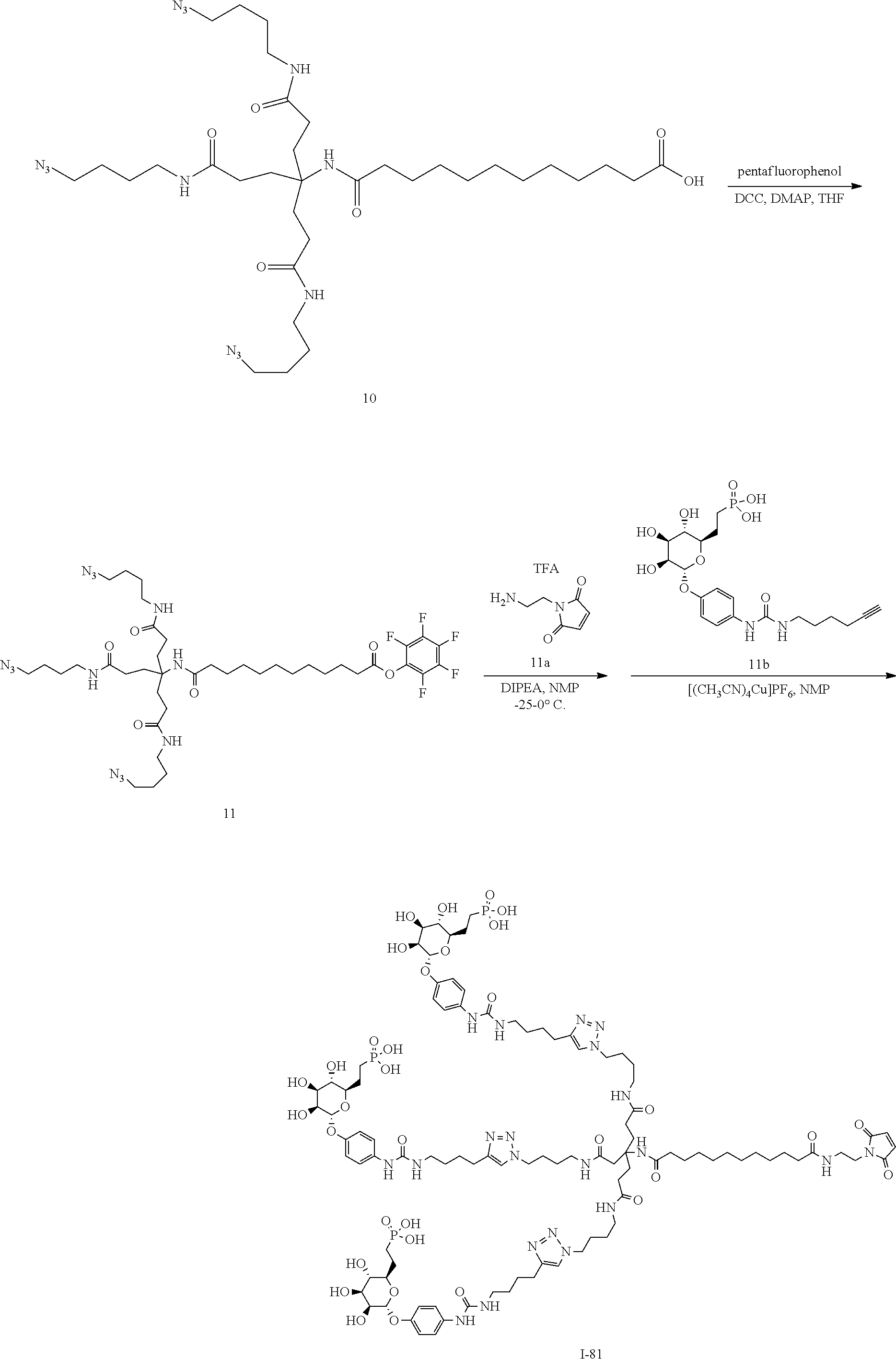

To a stirred mixture of di-tert-butyl 4-amino-4-(3-(tert-butoxy)-3-oxopropyl)heptanedioate (1, 1.00 eq, 1.01 g, 2.43 mmol) in 1,4-dioxane (10 mL) at 0° C. was added 1 M sodium carbonate in water (1.50 eq, 3.6 mL, 3.65 mmol) and then a solution of FMOC-Cl (1.20 eq, 755 mg, 2.92 mmol) in 1,4-dioxane (4 mL). The cold bath was removed and the resulting mixture was stirred vigorously at room temperature for 2 h. The reaction mixture was partitioned between ethyl acetate and brine. The organics were dried over magnesium sulfate, filtered, concentrated on a rotary evaporator, and purified via silica gel chromatography (0-30% ethyl acetate in hexanes) to afford (2) as a white foam-solid. Yield: 1.50 g, 97%; LCMS m/z 660.6 [M+Na]+; ¹H NMR (300 MHz, Chloroform-d) δ 7.76 (d, J=7.4 Hz, 2H), 7.59 (d, J=7.4 Hz, 2H), 7.40 (t, J=7.5 Hz, 2H), 7.31 (t, J=7.4 Hz, 2H), 5.01 (s, 1H), 4.36 (d, J=6.2 Hz, 2H), 4.18 (t, J=6.5 Hz, 1H), 2.25-2.12 (m, 6H), 1.98-1.83 (m, 6H), 1.43 (s, 27H).

To a stirred solution of (2, 1.00 eq, 1.50 g, 2.35 mmol) in DCM (10 mL) at 0° C. was added water (0.5 mL) and then TFA (3 mL). The resulting mixture was allowed to warm to room temperature and then stirred at room temperature for 18 h. More TFA (2 mL) was added and stirring at room temperature was continued for another 26 h. Volatiles were removed on a rotary evaporator. The residue was concentrated to dryness twice from dry toluene and then dried under high vacuum to afford (3) as a white solid. Yield: 1.19 g. LCMS 470.4 m/z [M+1]+; ¹H NMR (300 MHz, DMSO-d₆ with D₂O) δ 7.86 (d, J=7.5 Hz, 2H), 7.68 (d, J=7.5 Hz, 2H), 7.39 (t, J=7.4 Hz, 2H), 7.30 (t, J=7.9 Hz, 2H), 4.28-4.11 (m, 3H), 2.19-2.00 (m, 6H), 1.87-1.66 (m, 6H).

(3, 1.00 eq, 549 mg, 1.17 mmol), 4-dimethylaminopyridine (0.0200 eq, 2.9 mg, 0.0234 mmol), N,N′-dicyclohexylcarbodiimide (3.30 eq, 796 mg, 3.86 mmol), pentafluorophenol (3.50 eq, 753 mg, 4.09 mmol), and DMF (2.5 mL) were combined in a scintillation vial with a stirbar, capped, and stirred at room temperature for 4 h. More N,N′-dicyclohexylcarbodiimide (482 mg, 2.34 mmol) and pentafluorophenol (430 mg, 2.34 mmol) in DMF (1 mL) was added and the resulting mixture was capped and stirred at room temperature for 2 h. The reaction mixture was diluted with diethyl ether and filtered. The filtrate was washed three times with brine, dried over magnesium sulfate, filtered, concentrated on a rotary evaporator, and purified via silica gel chromatography (0-50% ethyl acetate in hexanes) to afford (4) and pentafluorophenol as a light yellow oil. Yield: 1.54 g. This material was taken on to the next step without further purification.

4-Azidobutan-1-amine (4a, 0.5 M in mTBE) (4.00 eq, 8.7 mL, 4.34 mmol) was added to a stirred solution of (4, 1.00 eq, 1.50 g, 1.09 mmol) in THF (10 mL) at room temperature. The resulting clear solution was capped and stirred at room temperature for 2 h. Most of the volatiles were removed on a rotary evaporator at room temperature. The residue was loaded onto a silica gel loading column with dichloromethane and purified via silica gel chromatography (0-100% ethyl acetate in dichloromethane) then (0-10% methanol in dichloromethane) to afford (5) as a colorless waxy solid. Yield: 624 mg, 76%; LCMS m/z 758.6 [M+1]+; H NMR (300 MHz, Chloroform-d) δ 7.77 (d, J=7.5 Hz, 2H), 7.60 (d, J=7.4 Hz, 2H), 7.41 (t, J=7.4 Hz, 2H), 7.31 (t, J=7.4 Hz, 2H), 6.08 (bs, 3H), 5.67 (bs, 1H), 4.37 (d, J=7.0 Hz, 2H), 4.18 (t, J=6.7 Hz, 1H), 3.34-3.13 (m, 12H), 2.24-2.09 (m, 6H), 2.04-1.85 (m, 6H), 1.66-1.47 (m, 12H).

Diethylamine (20.0 eq, 1.7 mL, 16.3 mmol) was added to a stirred solution of (5, 1.00 eq, 619 mg, 0.817 mmol) in methanol (8 mL). The resulting clear solution was capped and stirred at room temperature for 16 h. Volatiles were removed on a rotary evaporator. Methanol (10 mL) was added and volatiles were removed on a rotary evaporator again. This was repeated again to drive off diethylamine. The residue was taken up in methanol and loaded onto a 5 g Strata X-C ion exchange column from Phenomenex. The column was eluted sequentially with acetonitrile, methanol, and then 5% ammonium hydroxide in methanol. Fractions containing the desired product were combined, concentrated on a rotary evaporator and dried under high vacuum to afford (6) at 90% purity as a yellow oil. Yield: 483 mg, 99%; LCMS m/z 536.8 [M+1]+; ¹H NMR (300 MHz, Chloroform-d) δ 6.33 (t, J=5.8 Hz, 3H), 3.48 (s, 2H), 3.36-3.17 (m, 12H), 2.33-2.12 (m, 6H), 1.74-1.51 (m, 18H).

To a stirred solution of 12-(tert-butoxy)-12-oxododecanoic acid (7, 1.00 eq, 975 mg, 3.40 mmol) in DCM (7 mL) at room temperature under nitrogen was added DMF (5 microliters) and then oxalyl chloride (2 M in methylene chloride) (1.15 eq, 2.0 mL, 3.91 mmol). The resulting clear solution was stirred at room temperature under nitrogen for 1 h. Vigorous bubbling was observed. More oxalyl chloride (2 M in methylene chloride) (1.0 mL, 2.0 mmol) was added and the resulting mixture was stirred at room temperature under nitrogen for 30 min and then volatiles were removed on a rotary evaporator. The residue was dried under high vacuum to afford a yellow oil which was used in the next step without purification.

A solution of 4-amino-N1,N7-bis(4-azidobutyl)-4-(3-((4-azidobutyl)amino)-3-oxopropyl)heptanediamide (6, 1.00 eq, 707 mg, 1.19 mmol) and N,N-diisopropylethylamine (6.00 eq, 1.2 mL, 7.13 mmol) in DCM (4 mL) was added to a stirred solution of tert-butyl 12-chloro-12-oxododecanoate (8, 3.00 eq, 1.09 g, 3.56 mmol) in DCM (4 mL) at 0° C. under nitrogen. The resulting yellow solution was capped and stirred at room temperature for 30 min. Volatiles were removed on a rotary evaporator. The residue was taken up in acetic acid, and purified via reverse-phase flash chromatography (10-100% acetonitrile in water with 0.1% formic acid). Fractions containing the desired product were combined and concentrated at 30° C. on a rotary evaporator and the residue was dried under high vacuum to afford (9) as a colorless oil. Yield: 596 mg, 62%; LCMS m/z 804.8 [M+1]⁺.

(9, 1.00 eq, 592 mg, 0.736 mmol) was dissolved with stirring in DCM (4 mL) and then cooled to 0° C. Water (2 drops) was added and then TFA (2 mL) was added slowly down the side of the flask. The cold bath was removed and the resulting clear solution was stirred at room temperature for 1 h 20 min. Volatiles were removed on a rotary evaporator. The residue was taken up in acetic acid and purified via reverse-phase flash chromatography (10-100% acetonitrile in water with 0.1% formic acid). Fractions containing the desired product were combined, concentrated on a rotary evaporator, and dried under high vacuum to afford (10) as a colorless oil. Yield: 440 mg, 80%; LCMS m/z 748.7 [M+1]+; ¹H NMR (300 MHz, Chloroform-d) δ 7.13 (bs, 1H), 6.68 (bs, 3H), 3.37-3.16 (m, 12H), 2.38-2.20 (m, 8H), 2.15 (t, J=7.4 Hz, 2H), 2.08-1.96 (m, 6H), 1.72-1.49 (m, 16H), 1.41-1.18 (m, 12H).

To a stirred solution of (10, 1.00 eq, 436 mg, 0.583 mmol) in THF (2.5 mL) was added sequentially: N,N′dicyclohexylcarbodiimide (1.50 eq, 180 mg, 0.874 mmol), a solution of 2,3,4,5,6-pentafluorophenol (1.50 eq, 161 mg, 0.874 mmol) in THF (1 mL), and then 4-dimethylaminopyridine (0.0200 eq, 1.4 mg, 0.0117 mmol). The resulting mixture was capped and stirred at room temperature for 1.5 h. More N,N′dicyclohexylcarbodiimide (107 mg, 0.52 mmol) was added and stirring at room temperature was continued for another 21.5 h. The reaction mixture was diluted with diethyl ether and filtered. The filtrate was concentrated on a rotary evaporator. The residue was taken up in acetic acid and purified via reverse-phase flash chromatography (10-100% acetonitrile in water with 0.1% formic acid). Fractions containing the desired product were combined and lyophilized to dryness to afford (11) as a colorless wax. Yield: 431 mg, 81%; LCMS m/z 914.7 [M+1]+; ¹H NMR (300 MHz, Chloroform-d) δ 7.18 (bs, 1H), 6.14 (bs, 3H), 3.38-3.14 (m, 12H), 2.66 (t, J=7.4 Hz, 2H), 2.30-1.92 (m, 14H), 1.83-1.68 (m, 2H), 1.68-1.49 (m, 14H), 1.45-1.20 (m, 12H).

A solution of 1-(2-aminoethyl)-1H-pyrrole-2,5-dione TFA salt (11a, 1.00 eq, 6.8 mg, 0.0268 mmol) and N,N-diisopropylethylamine (1.30 eq, 0.0061 mL, 0.0348 mmol) in NMP (0.3 mL) was added to a stirred solution of (11, 1.00 eq, 24.5 mg, 0.0268 mmol) in DMF (0.3 mL) at −25° C. The resulting mixture was capped, stirred, and allowed to slowly warm to room temperature over 30 min. After warming to room temperature (I1b, 3.20 eq, 40.5 mg, 0.0858 mmol) was added. The resulting solution was stirred at room temperature for 3 min and then tetrakis(acetonitrile)copper(I) hexafluorophosphate (7.50 eq, 74.9 mg, 0.201 mmol) was added. The resulting light yellow solution was capped and stirred at room temperature for 25 min. Slowly turned more green-colored. The reaction mixture was diluted with a mixture of NMP and acetic acid, filtered, and purified via preparatory HPLC (10-50% acetonitrile in water with 0.1% TFA). Fractions containing the desired product were combined and lyophilized to dryness to afford (I-81) as a white solid. Yield: 14.1 mg, 23%; ¹H NMR (300 MHz, DMSO-d₆ with D₂O) δ 7.77 (s, 3H), 7.29-7.17 (m, 6H), 6.94-6.82 (m, 8H), 5.24 (s, 3H), 4.24 (t, J=6.8 Hz, 6H), 3.84-3.77 (m, 3H), 3.65-3.54 (m, 3H), 3.45-2.88 (m, 20H), 2.63-2.54 (m, 6H), 2.05-1.03 (m, 70H).

### Example 82: Synthesis of Compound I-82

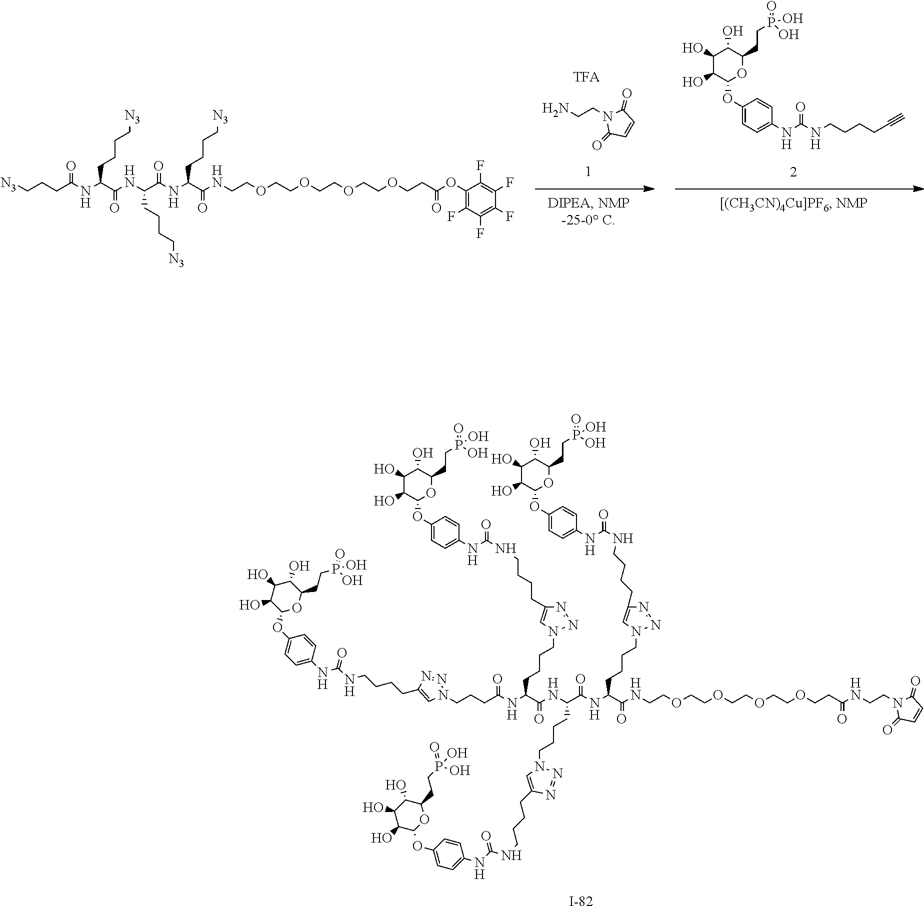

Compound 1-82 was prepared using similar methods. Yield: 22.8 mg, 40%; ¹H NMR (300 MHz, DMSO-d₆ with D₂O) δ 7.81-7.70 (m, 4H), 7.29-7.17 (m, 8H), 6.94-6.82 (m, 10H), 5.24 (s, 4H), 4.31-4.05 (m, 11H), 3.84-3.75 (m, 4H), 3.67-3.55 (m, 4H), 3.54-2.94 (m, 35H), 2.59-2.53 (m, 8H), 2.19 (t, J=6.0 Hz, 2H), 2.15-2.04 (m, 2H), 2.04-1.81 (m, 6H), 1.79-1.04 (m, 46H).

### Example 83: Synthesis of Compound I-83

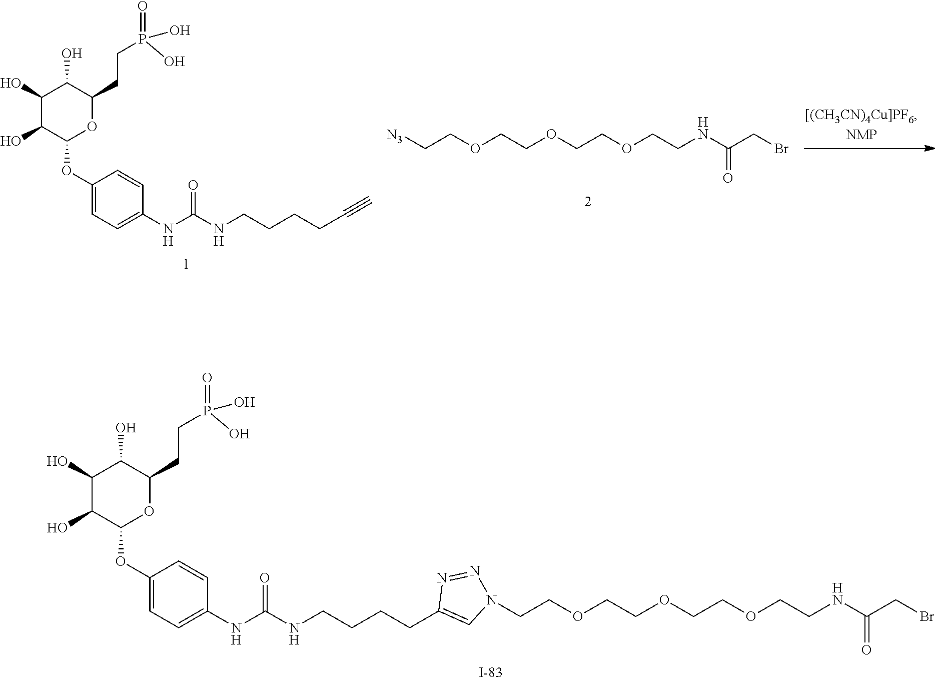

Compound I-83 was prepared using similar methods. Yield: 26.9 mg, 62.6%; LCMS m/z 813.4 [M+1]+; ¹H NMR (300 MHz, DMSO-d₆ with D₂O) δ 7.78 (s, 1H), 7.24 (d, J=7.7 Hz, 2H), 6.90 (d, J=7.7 Hz, 2H), 5.29-5.21 (m, 1H), 4.50-4.38 (m, 2H), 3.84-3.74 (m, 3H), 3.64-3.57 (m, 1H), 3.55-2.96 (m, 18H), 2.60 (t, J=7.6 Hz, 2H), 1.98-1.84 (m, 1H), 1.63-1.37 (m, 5H), 1.30-1.09 (m, 2H).

### Example 84: Synthesis of Compound I-84

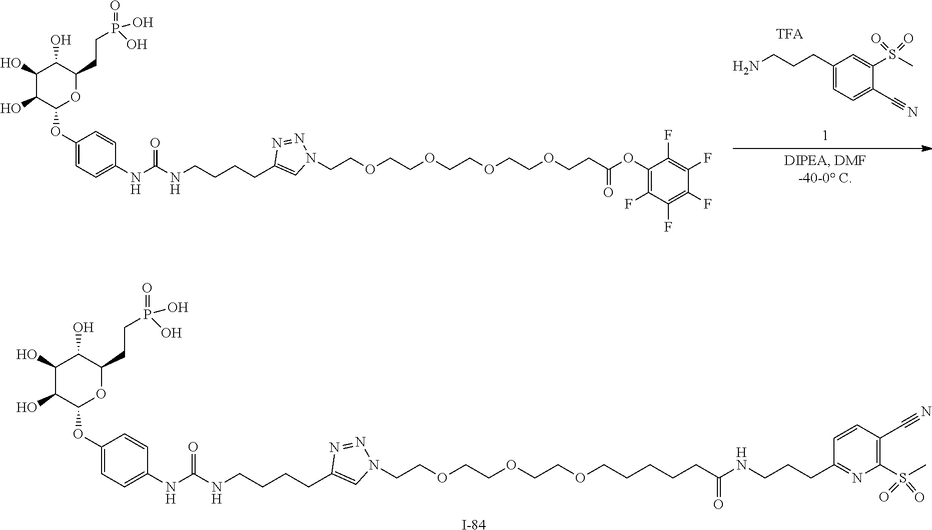

Compound I-84 (24 mg, 0.025 mmol, 77% yield) was prepared using similar methods. LCMS m/z 985.6 [M+1]+; ¹H NMR (300 MHz, DMSO-d6+D₂O) δ 8.43-8.36 (m, 1H), 7.76 (s, 1H), 7.75-7.68 (m, 1H), 7.23 (d, J=7.0 Hz, 2H), 6.89 (d, J=9.0 Hz, 2H), 5.28-5.20 (m, 1H), 4.47-4.37 (m, 2H), 3.84-3.78 (m, 1H), 3.78-3.69 (m, 2H), 3.65-3.50 (m, 3H), 3.48-3.27 (m, 17H), 3.13-2.99 (m, 5H), 2.87 (t, J=7.5 Hz, 2H), 2.59 (t, J=7.4 Hz, 2H), 2.26 (t, J=6.2 Hz, 2H), 1.98-1.76 (m, 3H), 1.62-1.37 (m, 5H), 1.28-1.15 (m, 1H).

### Example 85: Synthesis of Compound I-85

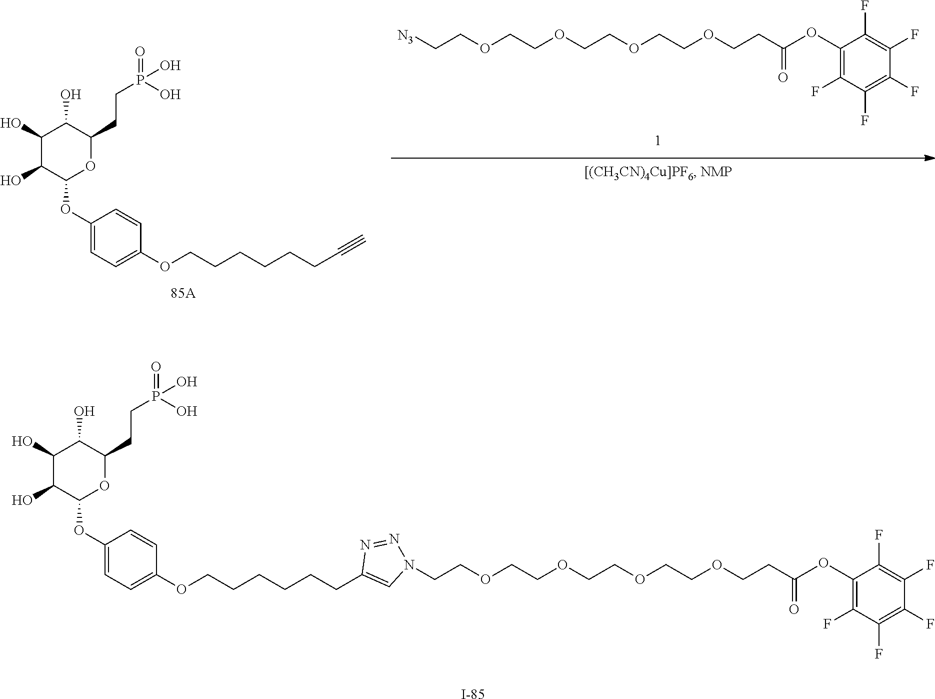

Compound I-85 was prepared using similar methods. Yield: 39.8 mg, 55%; LCMS m/z 916.5 [M+1]+; ¹H NMR (300 MHz, DMSO-d₆ with D₂O) δ 7.75 (s, 1H), 6.92 (d, J=8.1 Hz, 2H), 6.80 (d, J=8.5 Hz, 2H), 5.19 (s, 1H), 4.41 (t, J=4.8 Hz, 2H), 3.85-3.67 (m, 7H), 3.64-3.53 (m, 1H), 3.54-3.37 (m, 12H), 3.31 (d, J=6.3 Hz, 2H), 2.93 (t, J=5.9 Hz, 2H), 2.56 (t, J=7.3 Hz, 2H), 1.99-1.80 (m, 1H), 1.70-1.04 (m, 11H).

### Example 86: Synthesis of Compound I-86

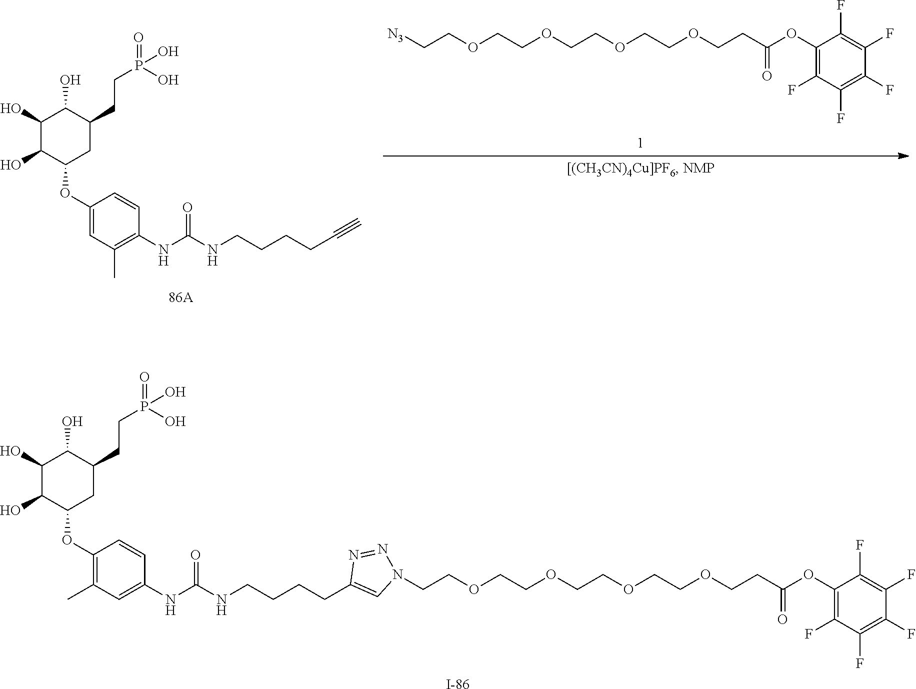

Compound I-86 was prepared using similar methods. Yield: 37.4 mg, 65%; LCMS m/z 944.5 [M+1]+; ¹H NMR (300 MHz, DMSO-d₆ with D₂O) δ 7.77 (s, 1H), 7.43 (d, J=8.8 Hz, 1H), 6.81 (s, 1H), 6.75 (d, J=8.8 Hz, 1H), 5.23 (s, 1H), 4.42 (t, J=5.5 Hz, 2H), 3.97-3.68 (m, 5H), 3.64-3.56 (m, 1H), 3.54-3.38 (m, 12H), 3.35-3.27 (m, 2H), 3.04 (t, J=6.6 Hz, 2H), 2.98-2.89 (m, 2H), 2.59 (t, J=7.3 Hz, 2H), 2.09 (s, 3H), 1.98-1.81 (m, 1H), 1.69-1.34 (m, 6H), 1.31-1.10 (m, 1H).

### Example 87: Synthesis of Compound I-87

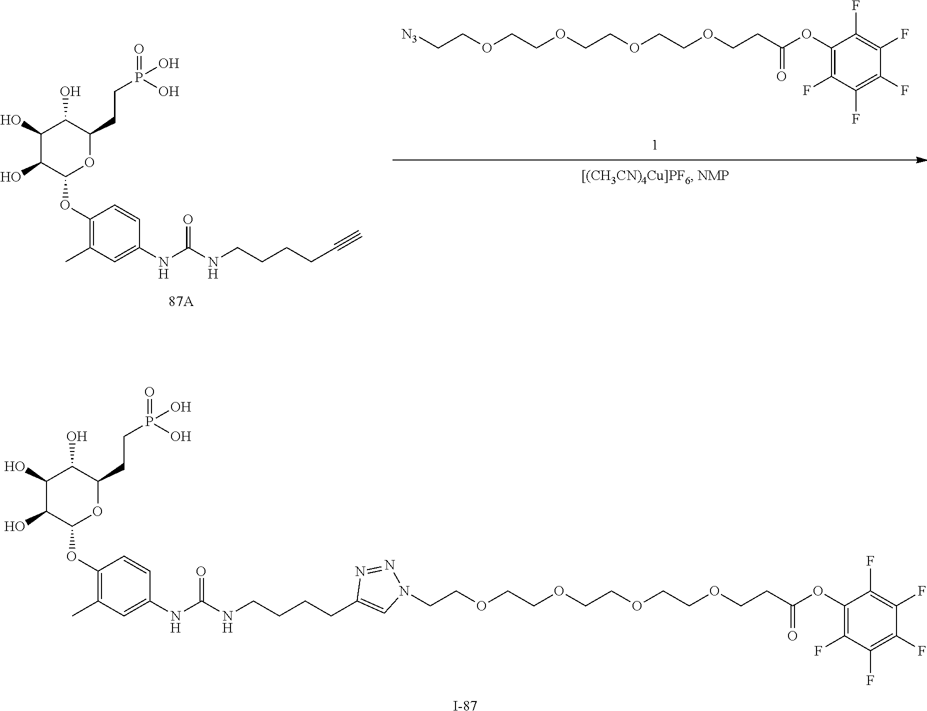

Compound I-87 was prepared using similar methods. Yield: 39.8 mg, 69%; LCMS m/z 944.5 [M+1]+; ¹H NMR (300 MHz, DMSO-d₆ with D₂O) δ 7.77 (s, 1H), 7.12 (s, 1H), 7.06 (d, J=8.7 Hz, 1H), 6.88 (d, J=8.8 Hz, 1H), 5.22 (s, 1H), 4.41 (t, J=5.1 Hz, 2H), 3.97-3.68 (m, 5H), 3.67-3.59 (m, 1H), 3.55-3.39 (m, 12H), 3.37-3.22 (m, 2H), 3.02 (t, J=7.3 Hz, 2H), 2.94 (t, J=5.9 Hz, 2H), 2.59 (t, J=7.5 Hz, 2H), 2.08 (s, 3H), 1.98-1.82 (m, 1H), 1.71-1.33 (m, 6H), 1.30-1.09 (m, 1H).

### Example 88: Synthesis of Compound I-88

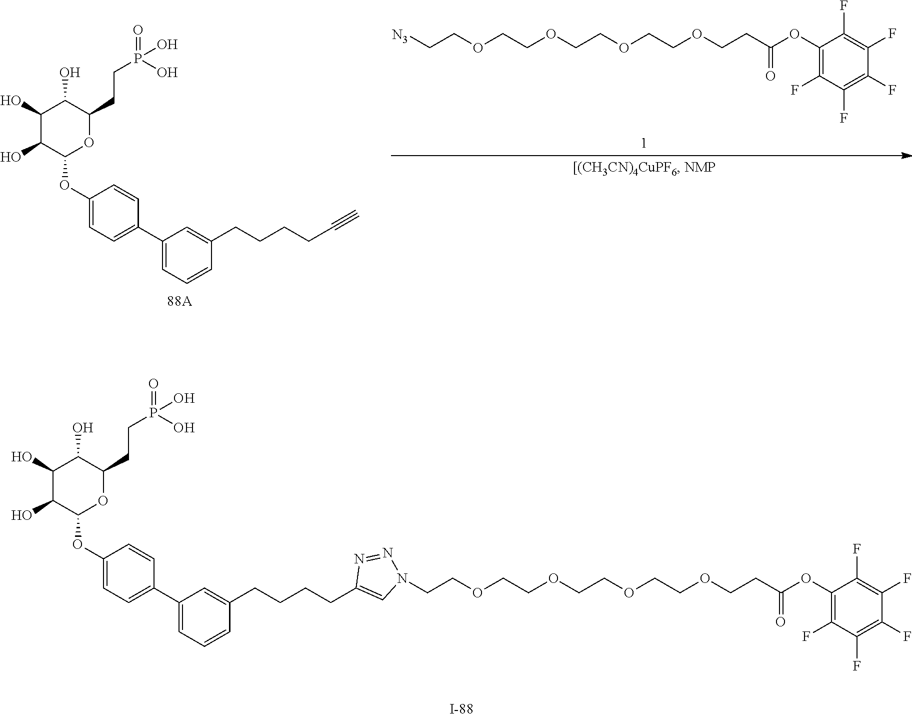

Compound I-88 was prepared using similar methods. Yield: 40.5 mg, 68%; LCMS 948.5 m/z [M+1]+; ¹H NMR (300 MHz, DMSO-d₆ with D₂O) δ 7.74 (s, 1H), 7.55 (d, J=8.3 Hz, 2H), 7.36 (s, 2H), 7.30 (t, J=7.6 Hz, 1H), 7.08 (d, J=8.0 Hz, 3H), 5.39 (s, 1H), 4.40 (s, 2H), 3.79-3.58 (m, 5H), 3.53-3.23 (m, 15H), 2.92 (t, J=5.8 Hz, 2H), 2.68-2.56 (m, 4H), 2.01-1.80 (m, 1H), 1.68-1.43 (m, 6H), 1.28-1.06 (m, 1H).

### Example 89: Synthesis of Compound I-89

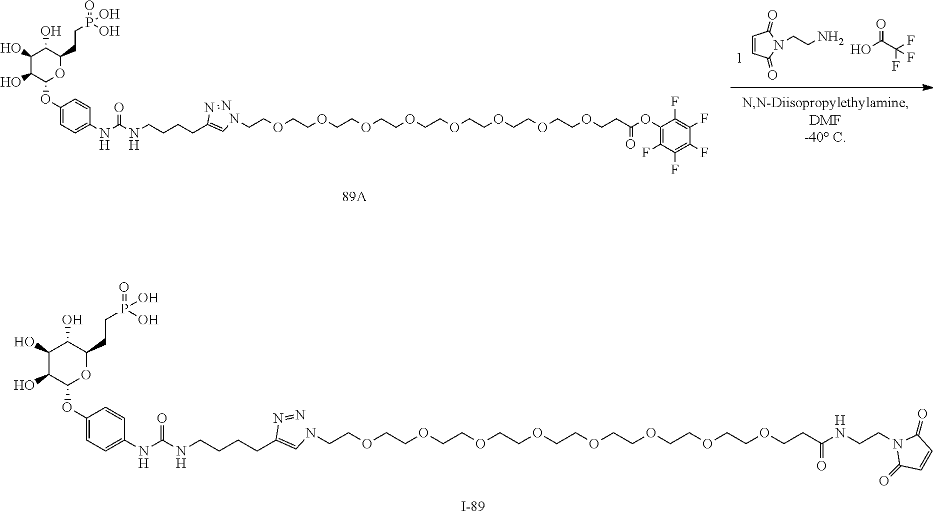

Compound I-89 was prepared using similar methods. Yield: 24.2 mg, 57%; LCMS m/z 1062.6 [M+1]+; ¹H NMR (300 MHz, DMSO-d₆ with D₂O) δ 7.78 (s, 1H), 7.24 (d, J=8.5 Hz, 2H), 6.99-6.79 (m, 4H), 5.24 (s, 1H), 4.42 (bs, 2H), 3.79-3.70 (m, 4H), 3.64-3.55 (m, 1H), 3.56-3.36 (m, 31H), 3.35-3.28 (m, 2H), 3.23-3.12 (m, 2H), 3.11-2.99 (m, 2H), 2.67-2.55 (m, 2H), 2.24-2.12 (m, 2H), 2.02-1.77 (m, 1H), 1.71-1.34 (m, 6H), 1.32-1.04 (m, 1H).

### Example 90: Synthesis of Compound I-90

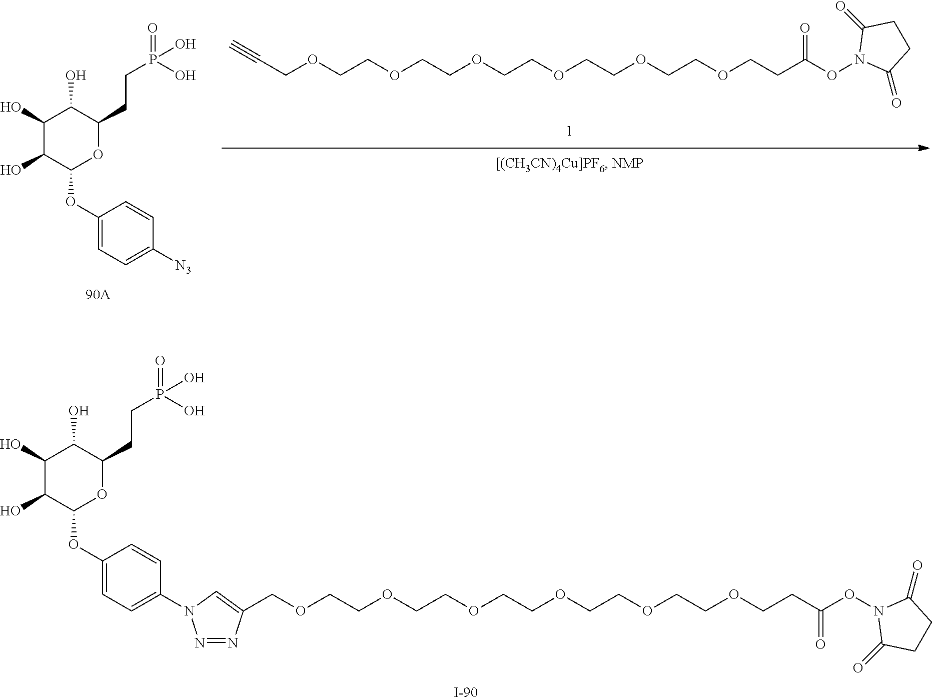

Compound I-90 was prepared using similar methods. Yield: 32.3 mg, 58%; LCMS m/z 821.5 [M+1]+; ¹H NMR (300 MHz, DMSO-d₆ with D₂O) δ 8.58 (s, 1H), 7.83-7.71 (m, 2H), 7.29-7.17 (m, 2H), 5.47 (s, 1H), 4.58 (s, 2H), 3.89-3.80 (m, 2H), 3.72-3.62 (m, 3H), 3.62-3.23 (m, 21H), 2.85 (t, J=5.9 Hz, 2H), 2.77 (s, 4H), 2.00-1.82 (m, 1H), 1.70-1.40 (m, 2H), 1.27-1.03 (m, 1H).

### Example 91: Synthesis of Compound I-91

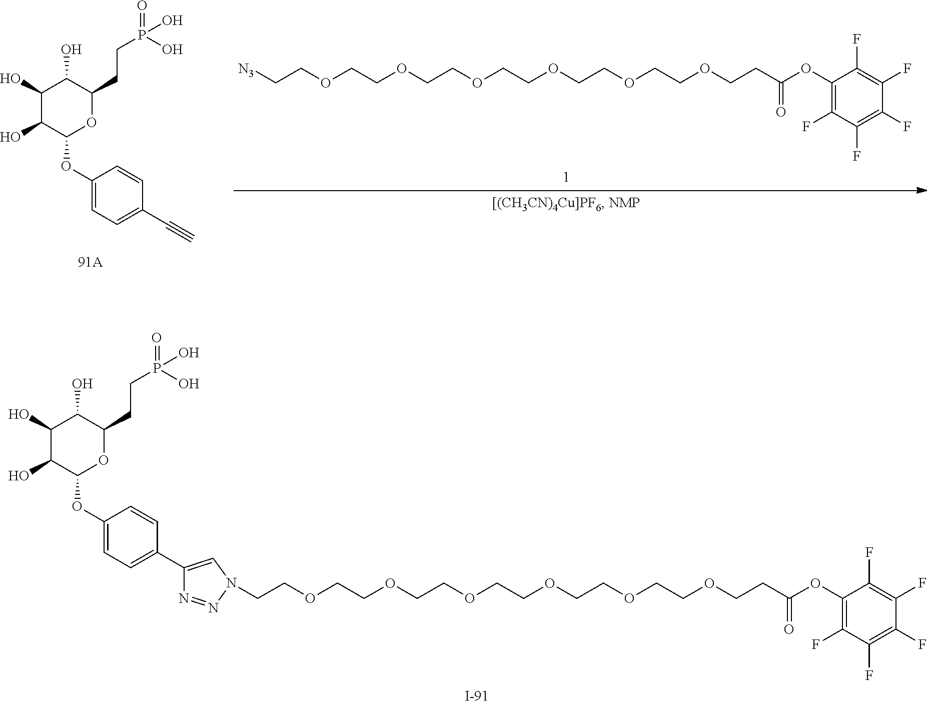

Compound I-91 was prepared using similar methods. Yield: 43.7 mg, 68%; LCMS m/z 904.5 [M+1]+; ¹H NMR (300 MHz, DMSO-d₆ with D₂O) δ 8.35 (s, 1H), 7.73 (d, J=8.0 Hz, 2H), 7.09 (d, J=8.3 Hz, 2H), 5.40 (s, 1H), 4.51 (t, J=5.0 Hz, 2H), 3.85-3.79 (m, 3H), 3.72 (t, J=5.8 Hz, 2H), 3.64 (dd, J=8.8, 3.4 Hz, 1H), 3.53-3.23 (m, 22H), 2.94 (t, J=5.8 Hz, 2H), 2.01-1.78 (m, 1H), 1.71-1.38 (m, 2H), 1.28-1.00 (m, 1H).

### Example 92: Synthesis of Compound I-92

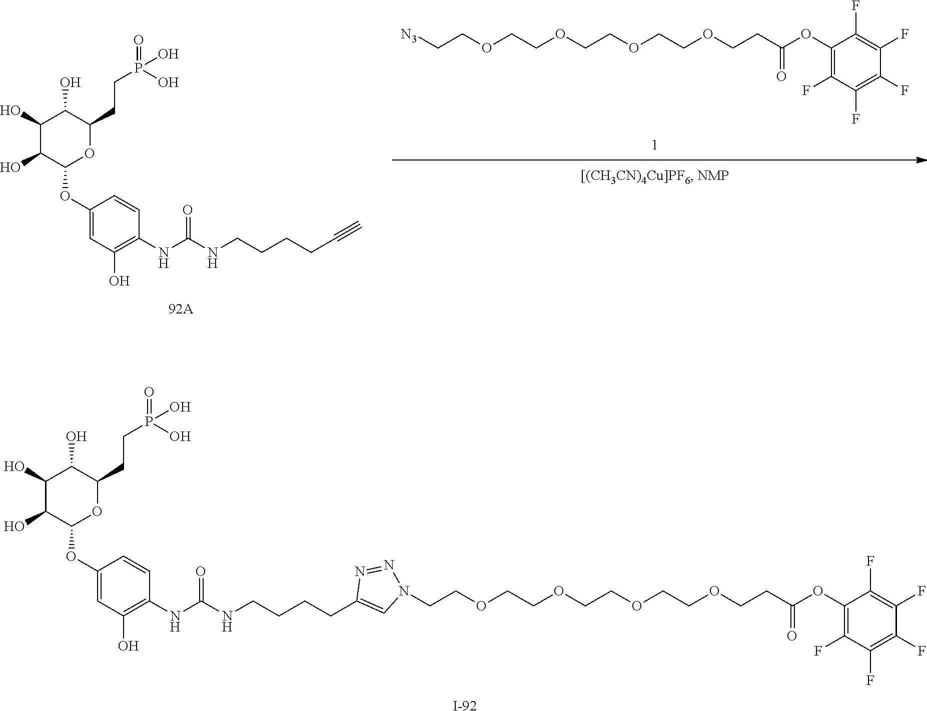

Compound I-92 was prepared using similar methods. Yield: 42.0 mg, 71%; LCMS m/z 946.5 [M+1]+; ¹H NMR (300 MHz, DMSO-d₆ with D₂O) δ 7.77 (s, 1H), 7.57 (d, J=8.8 Hz, 1H), 6.53-6.45 (m, 1H), 6.43-6.32 (m, 1H), 5.19 (s, 1H), 4.41 (t, J=5.0 Hz, 2H), 3.80-3.68 (m, 5H), 3.61-3.54 (m, 1H), 3.53-3.38 (m, 12H), 3.33-3.27 (m, 2H), 3.04 (t, J=6.9 Hz, 2H), 2.93 (t, J=5.8 Hz, 2H), 2.59 (t, J=7.4 Hz, 2H), 2.00-1.83 (m, 1H), 1.74-1.34 (m, 6H), 1.34-1.12 (m, 1H).

### Example 93: Synthesis of Compound I-93

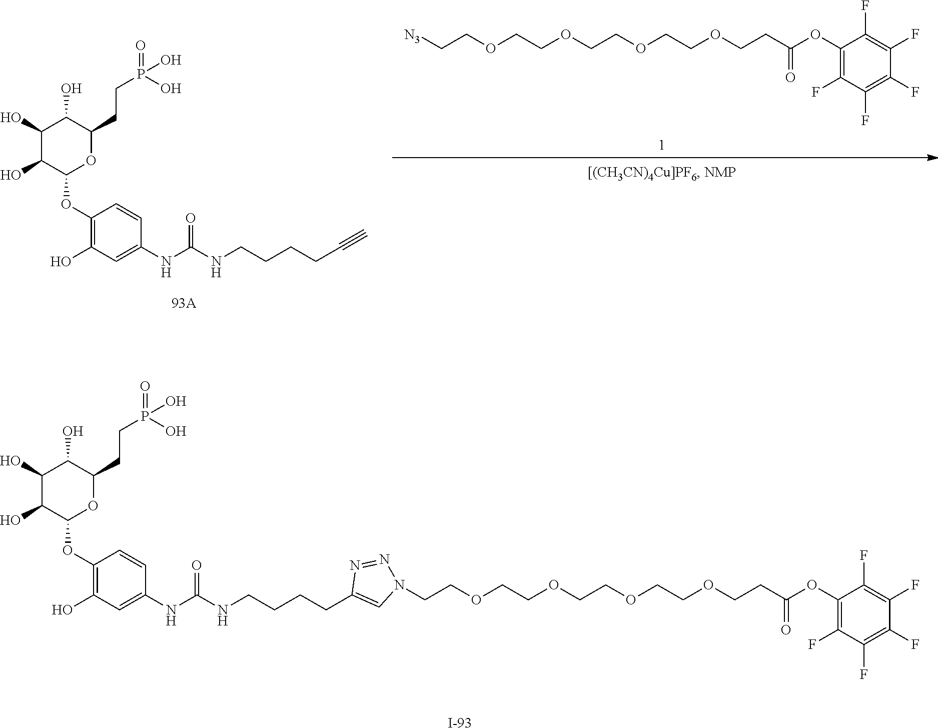

Compound I-93 was prepared using similar methods. Yield: 43.6 mg, 74%; LCMS m/z 946.5 [M+1]+; ¹H NMR (300 MHz, DMSO-d₆ with D₂O) δ 7.77 (s, 1H), 7.02-6.96 (m, 1H), 6.83 (d, J=8.7 Hz, 1H), 6.64-6.54 (m, 1H), 5.11 (s, 1H), 4.41 (t, J=5.2 Hz, 2H), 3.88-3.85 (m, 1H), 3.79-3.60 (m, 5H), 3.57-3.36 (m, 13H), 3.30 (t, J=9.4 Hz, 1H), 3.03 (t, J=6.7 Hz, 2H), 3.03-2.88 (m, 2H), 2.59 (t, J=7.4 Hz, 2H), 2.02-1.81 (m, 1H), 1.69-1.14 (m, 7H).

### Example 94: Synthesis of Compound I-94

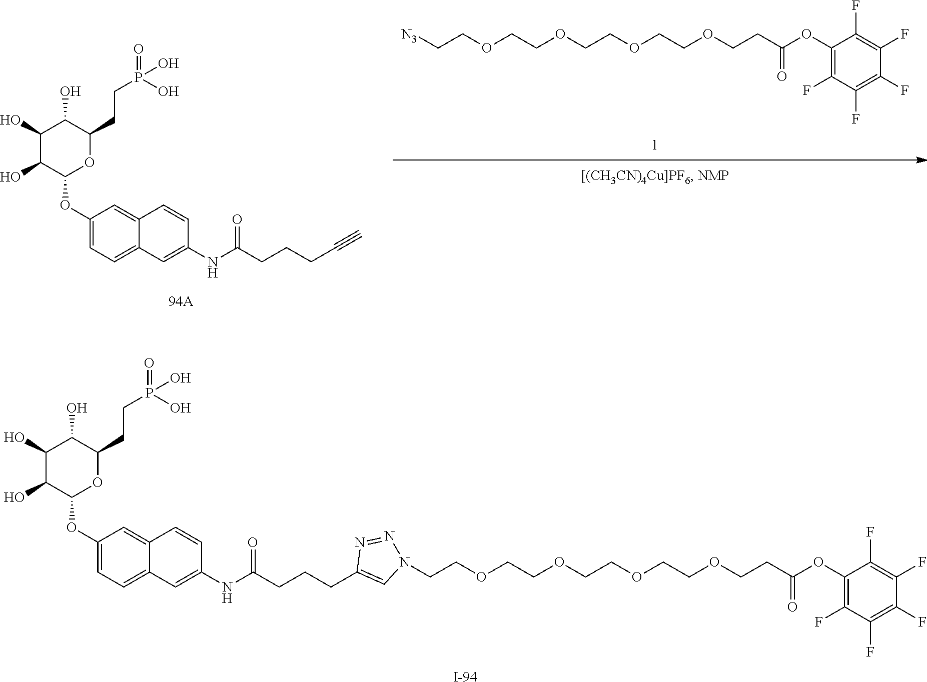

Compound I-94 was prepared using similar methods. Yield: 36.7 mg, 76%; LCMS m/z 951.5 [M+1]+; ¹H NMR (300 MHz, DMSO-d₆ with D₂O) δ 8.15 (s, 1H), 7.81 (s, 1H), 7.71 (d, J=8.9 Hz, 2H), 7.49 (d, J=8.9 Hz, 1H), 7.39 (d, J=2.4 Hz, 1H), 7.19 (dd, J=8.8, 2.4 Hz, 1H), 5.47 (s, 1H), 4.43 (t, J=5.0 Hz, 2H), 3.79-3.63 (m, 5H), 3.52-3.27 (m, 15H), 2.91 (t, J=5.8 Hz, 2H), 2.65 (t, J=7.6 Hz, 2H), 2.38 (t, J=7.4 Hz, 2H), 1.97-1.82 (m, 3H), 1.70-1.39 (m, 2H), 1.24-1.01 (m, 1H).

### Example 95: Synthesis of Compound I-95

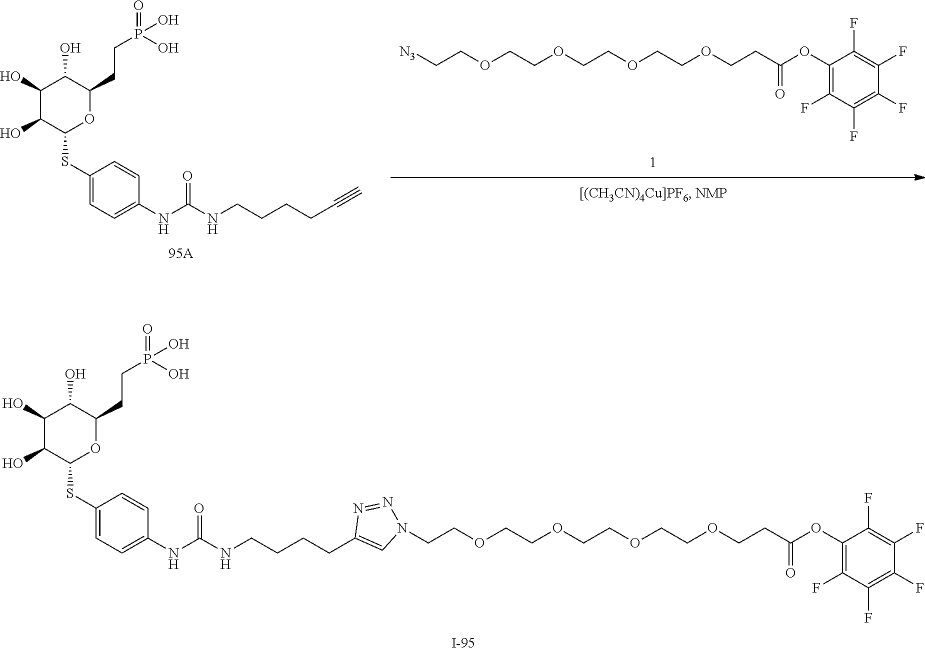

Compound I-95 was prepared using similar methods. Yield: 36.3 mg, 74%; LCMS m/z 946.5 [M+1]+; H NMR (300 MHz, DMSO-d₆ with D₂O) δ 7.77 (s, 1H), 7.36-7.23 (m, 4H), 5.14 (s, 1H), 4.42 (t, J=4.9 Hz, 2H), 3.90-3.64 (m, 6H), 3.55-3.37 (m, 13H), 3.32 (t, J=9.3 Hz, 1H), 3.06 (t, J=6.7 Hz, 2H), 2.95 (t, J=5.5 Hz, 2H), 2.59 (t, J=7.4 Hz, 2H), 2.04-1.87 (m, 1H), 1.64-1.27 (m, 7H).

### Example 96: Synthesis of Compound I-96

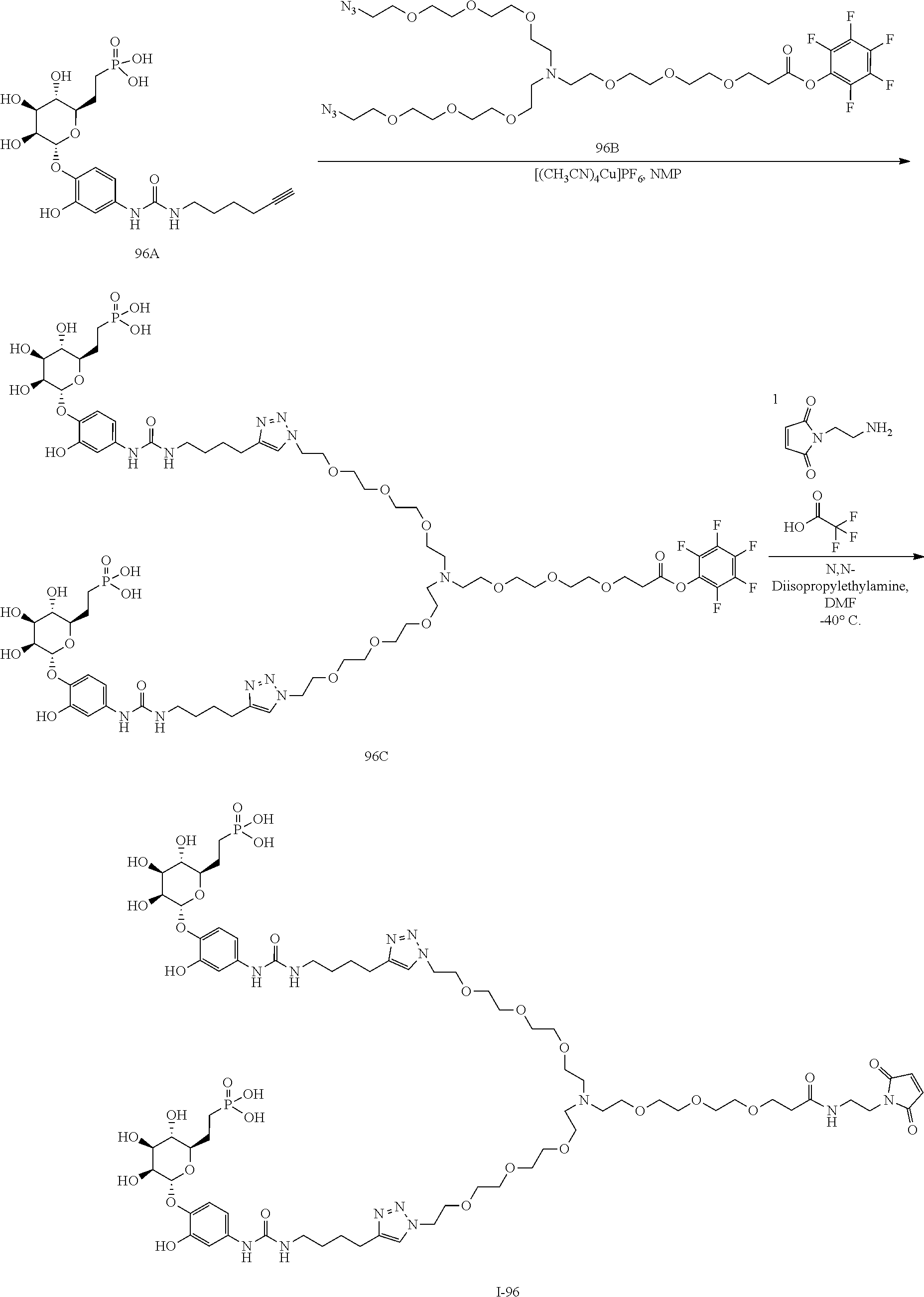

was prepared using similar methods I-96 was prepared using similar methods. Yield: 10.6

44%; LCMS m/z 1722.1 [M−1]-; H NMR (300 MHz, DMSO-d₆ with D₂O) δ 7.75 (s, 2H), 6.98 (s, 2H), 6.88-6.79 (m, 4H), 6.59 (d, J=8.6 Hz, 2H), 5.11 (s, 2H), 4.45-4.35 (m, 4H), 3.87 (bs, 2H), 3.76-3.60 (m, 12H), 3.55-3.25 (m, 38H), 3.20-3.11 (m, 2H), 3.08-2.96 (m, 4H), 2.63-2.54 (m, 4H), 2.25-2.14 (m, 2H), 1.97-1.82 (m, 2H), 1.65-1.12 (m, 14H).

### Example 97: Synthesis of Compound I-97

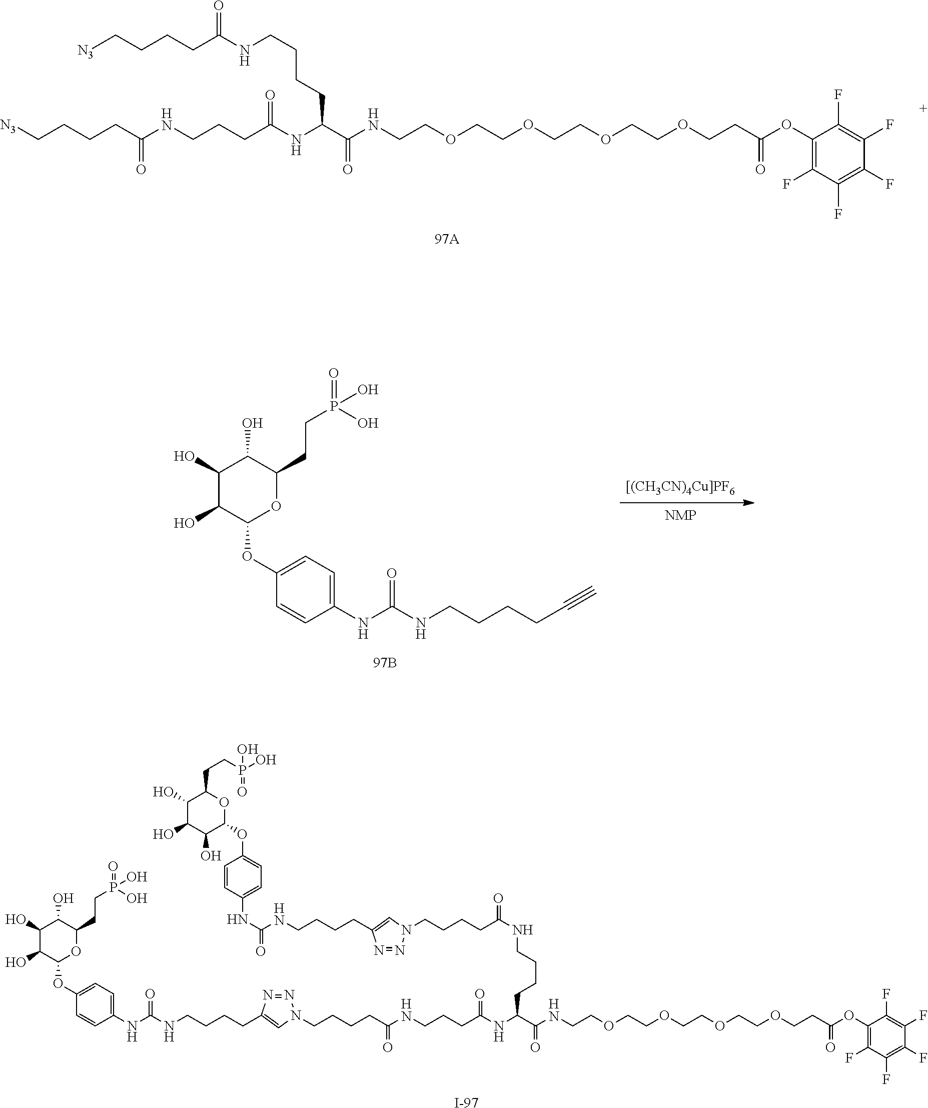

Compound I-97 was prepared using similar methods. Yield: 45 mg, 71%; LCMS m/z 1840.2 [M+1]+; ¹H NMR (300 MHz, DMSO-d₆ with D₂O) δ 7.84 (s, 2H), 7.30 (d, J=9.0 Hz, 4H), 6.95 (d, J=9.1 Hz, 4H), 5.30 (d, J=1.9 Hz, 2H), 4.31 (t, J=6.7 Hz, 4H), 4.18-4.13 (m, 1H), 3.88-3.64 (m, 4H), 3.61-3.32 (m, 18H), 3.30-2.93 (m, 14H), 2.68-2.61 (m, 4H), 2.20-2.06 (m, 5H), 2.03-1.90 (m, 1H), 1.84-1.17 (m, 32H).

### Example 98: Synthesis of Compound I-98

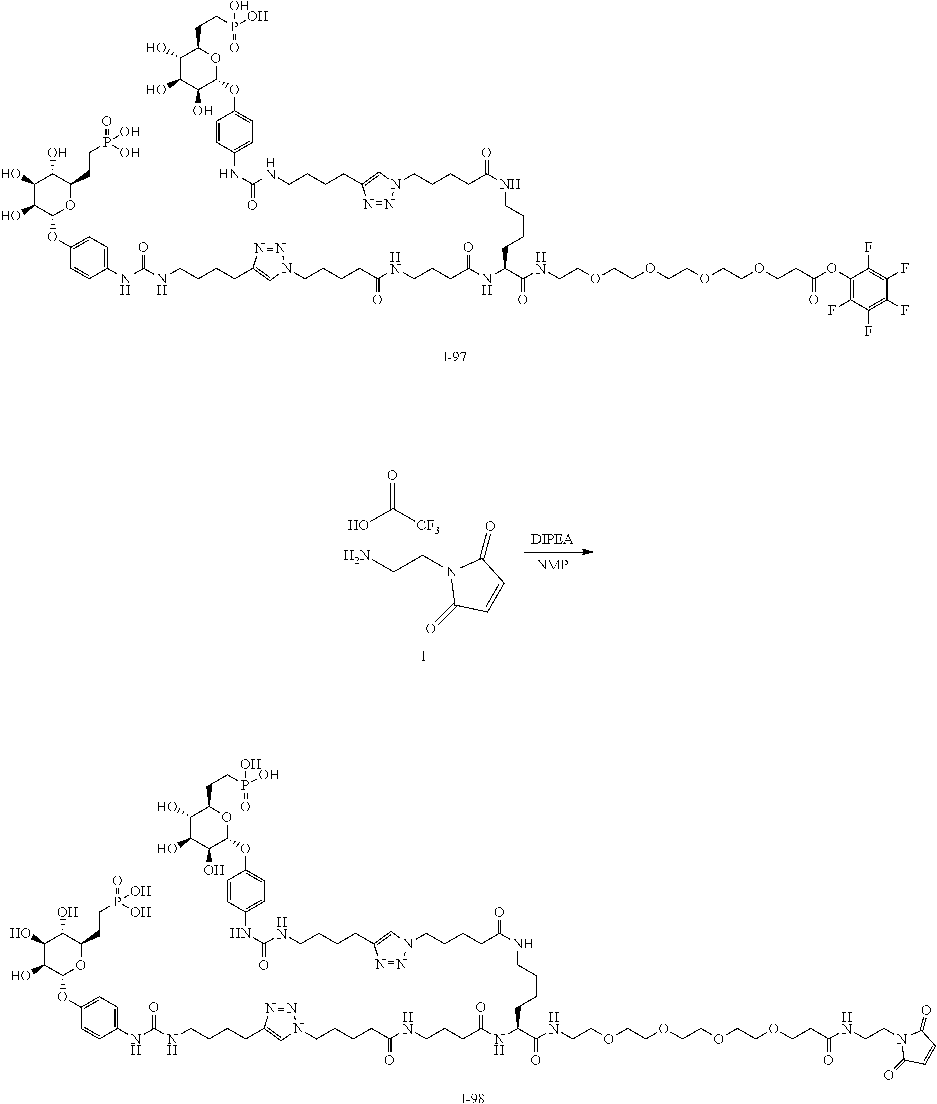

Compound I-98 was prepared using similar methods. Yield: 19 mg, 55%; LCMS m/z 1796.0 [M+1]+; ¹H NMR (300 MHz, DMSO-d₆ with D₂O) δ 7.75 (s, 2H), 7.21 (d, J=8.9 Hz, 4H), 6.91-6.80 (m, 6H), 5.21 (d, J=1.9 Hz, 2H), 4.22 (t, J=6.9 Hz, 4H), 4.11-4.04 (m, 1H), 3.61-3.53 (m, 2H), 3.51-3.23 (m, 22H), 3.18-2.89 (m, 14H), 2.60-2.51 (m, 4H), 2.16 (t, J=6.5 Hz, 2H), 2.09-1.83 (m, 6H), 1.77-1.10 (m, 32H).

### Example 99: Synthesis of Compound I-99

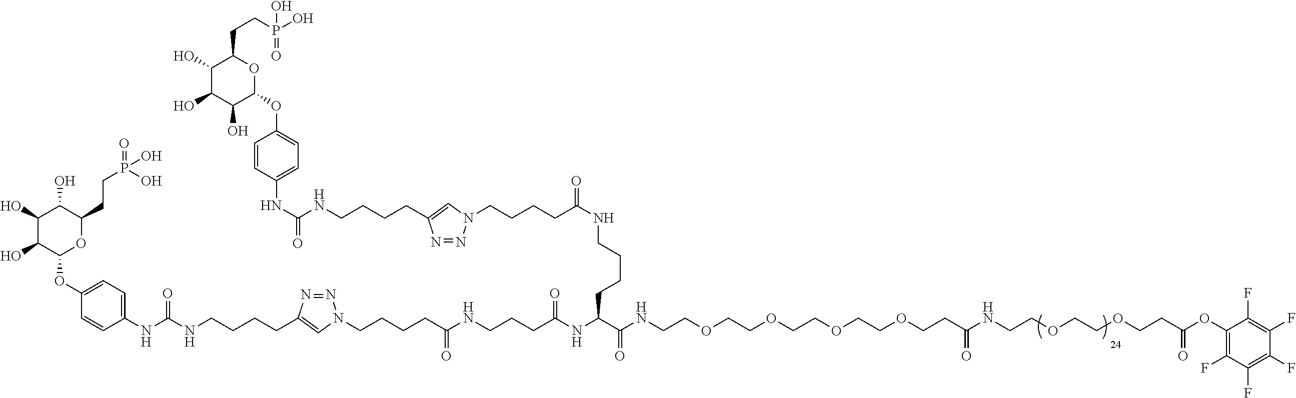

Compound I-99 was prepared from compound 97B according to methods similar to those described herein. Cpd. No. I-99 as a white solid. Yield: 99 mg; H NMR (300 MHz, DMSO-d₆ with D₂O) S 7.78 (s, 2H), 7.25 (d, J=8.5 Hz, 4H), 6.89 (d, J=8.5 Hz, 4H), 5.24 (s, 2H), 4.30-4.20 (m, 4H), 4.16-4.07 (m, 1H), 3.48 (d, J=20.2 Hz, 116H), 3.34 (dd, J=16.8, 6.0 Hz, 8H), 3.20-3.15 (m, 2H), 3.08-2.92 (m, 8H), 2.59 (t, J=7.4 Hz, 4H), 2.30 (t, J=6.4 Hz, 2H), 1.96-1.12 (m, 38H).

### Example 100: Synthesis of Compound I-100

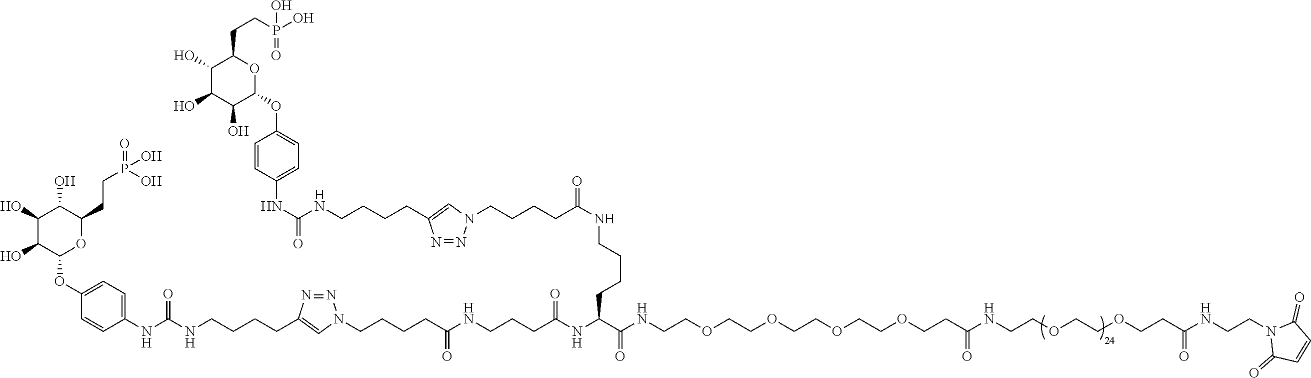

Compound I-100 was prepared from compound I-99 according to methods similar to those described herein. as a white solid. Yield: 27 mg; ¹H NMR (300 MHz, DMSO-d₆ with D₂O) δ 8.03 (br, 2H), 7.84 (s, 2H), 7.30 (d, J=8.6 Hz, 4H), 7.00-6.92 (m, 6H), 5.30 (s, 2H), 4.31 (t, J=6.9 Hz, 4H), 4.20-4.14 (m, 1H), 3.70-3.36 (m, 120H), 3.28-3.19 (m, 6H), 3.17-2.98 (m, 1OH), 2.65 (t, J=7.4 Hz, 4H), 2.36 (t, J=6.3 Hz, 2H), 2.27 (t, J=6.5 Hz, 2H), 2.19-2.08 (m, 6H), 2.02-1.18 (m, 32H).

### Example 101: Synthesis of Compound I-101B

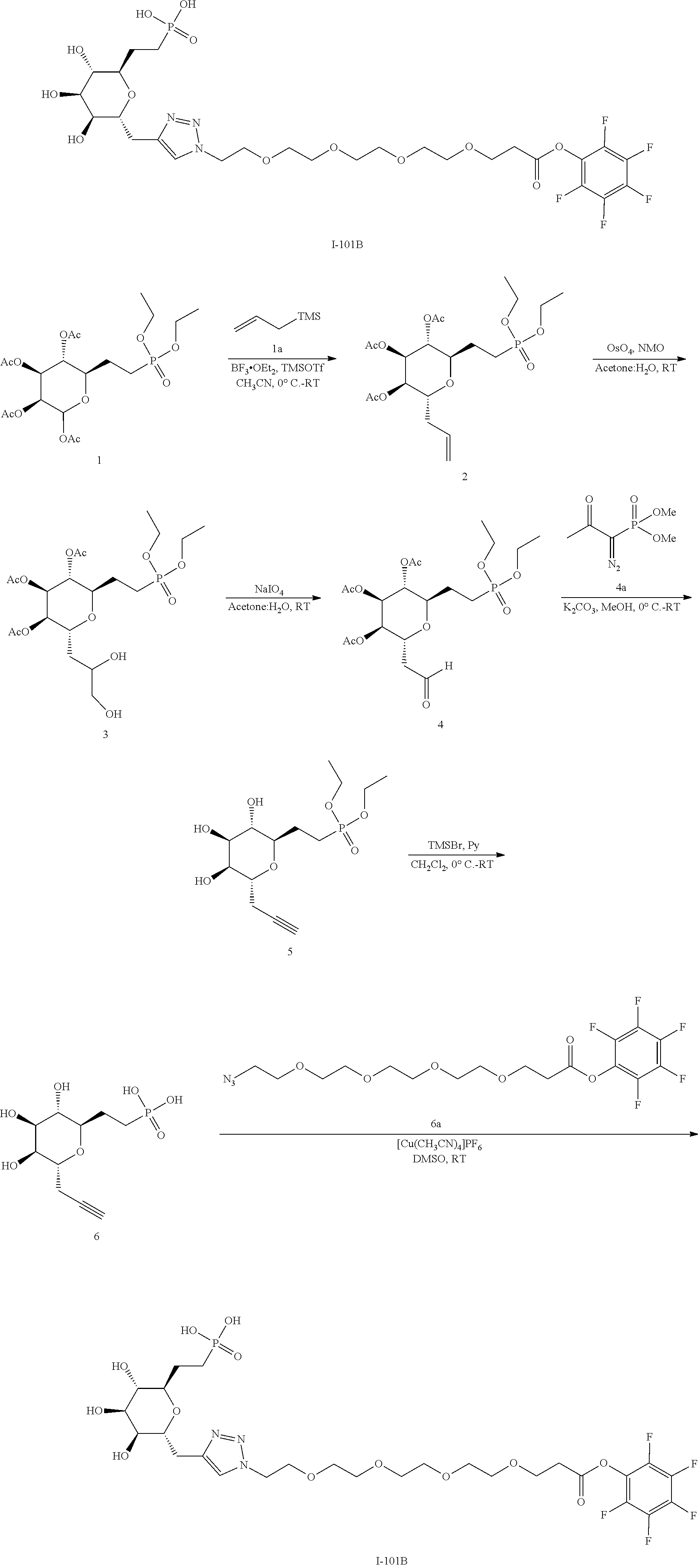

To a stirred solution of ((3S,4S,5R,6R)-6-(2-(diethoxyphosphoryl)ethyl)tetrahydro-2H-pyran-2,3,4,5-tetrayl tetraacetate (1, 1.0 eq., 4.3 g, 8.91 mmol) in acetonitrile (40 mL) was added allyltrimethylsilane (1a, 4.0 eq., 5.67 mL, 35.7 mmol) followed by boron trifluoride diethyl etherate (4.0 eq., 4.4 mL, 35.7 mmol) and trimethylsilyl trifluoromethanesulfonate (0.3 eq., 0.485 mL, 2.67 mmol), sequentially at 0° C. under nitrogen atmosphere. The reaction mixture was then stirred for 12 h at room temperature. After that, reaction mixture was poured into ice-cold saturated aqueous sodium bicarbonate solution and extracted with dichloromethane. Organic part was again washed with brine, dried over anhydrous sodium sulphate, concentrated and purified by silica gel column chromatography (using 10% methanol in dichloromethane) to give (2) as light yellow syrup. Yield: 3.48 g, 84.0%, LCMS m/z 465.0 [M+1]⁺.

N-Methylmorpholine N-oxide (1.5 eq., 0.397 g, 1.5 eq, 3.39 mmol) followed by osmium tetraoxide (0.1 eq, 1.44 mL, 0.226 mmol, 4.0 wt % in water) were added to a stirred solution of (2R,3R,4R,5R,6R)-2-allyl-6-(2-(diethoxyphosphoryl)ethyl)tetrahydro-2H-pyran-3,4,5-triyl triacetate (2, 1.0 eq, 1.05 g, 2.26 mmol) in acetone-water (5:1, 30.0 mL) at room temperature. After 2 h, TLC showed complete consumption of starting material and a lower spot generated (based on TLC observation). The mixture was extracted with ethylacetate (50 mL). The organic part was dried over anhydrous sodium sulfate, filtered and the solvent was removed in vacuo to give crude (3) which was directly used for next step.

To a stirred solution of crude (3, 1.2 g, 2.41 mmol) in a mixture of acetone: water (2:1, 20 mL) at 0° C., was added sodium periodate (2 eq, 1.03 g, 4.81 mmol) and then allowed to stir at room temperature. After being stirred at room temperature for 2 h, the TLC showed full consumption of starting material and a less polar new spot was generated on TLC. Then ethyl acetate was added to reaction mixture and extracted with ethyl acetate. The organic part was dried over anhydrous sodium sulfate, filtered and concentrated to give crude product which was then purified by flash column chromatography using 7-10% methanol in dichloromethane to give (4) as colorless syrup. Yield: 0.91 g, 81.0%. LCMS m/z 467.1 [M+1]⁺.

To a solution of (4, 1.00 eq, 0.91 g, 1.95 mmol) in methanol (25.0 mL) at 0° C., were added potassium carbonate (3 eq., 0.809 g, 5.85 mmol), dimethyl (1-diazo-2-oxopropyl)phosphonate (4a, 2 eq., 0.75 g, 3.9 mmol) and reaction mixture was stirred at room temperature for 3 h. TLC showed formation of polar spot. The volatiles were then evaporated in vacuo to get a crude reaction mass which was purified by silica gel flash column chromatography using 10-12% methanol in dichloromethane to give (5) as colorless syrup. Yield: 0.35 g, 53.3%. LCMS m/z 337.0 [M+1]⁺.

To a stirred solution of (5, 1.0 eq, 0.35 g, 1.04 mmol) in dichloromethane (15.0 mL), were added pyridine (10.0 eq, 0.838 mL, 10.4 mmol) and bromotrimethylsilane (10.0 eq, 1.37 mL, 10.4 mmol) at 0° C. and reaction mixture was allowed to stir at room temperature. After 16 h, volatiles were evaporated and the crude mass was purified by prep-HPLC (using 40-60% acetonitrile in water with 0.1% TFA, to elute from a C18 column). The fractions containing desired compound were collected and lyophilized to give (6) as a off-white solid. Yield: 0.101 g, 34.64% LCMS m/z 281.0 [M+1]⁺.

A solution of 2,3,4,5,6-pentafluorophenyl 1-azido-3,6,9,12-tetraoxapentadecan-15-oate (1.1 eq, 0.156 g, 0.342 mmol) in dimethyl sulfoxide (3 mL), (6, 1.0 eq, 0.087 g, 0.310 mmol), tetrakis(acetonitrile)copper(I) hexafluorophosphate (2.8 eq., 0.324 g, 0.869 mmol) were added and reaction mixture was stirred at room temperature for 30 min. Thereafter, acetic acid (0.5 mL) was added and reaction mixture was diluted with acetonitrile and purified by prep HPLC (23-41% acetonitrile in water with 0.1% TFA). Fractions containing the desired product were combined and lyophilized to dryness to afford (2-((2R,3S,4R,5S,6R)-3,4,5-trihydroxy-6-((1-(15-oxo-15-(perfluorophenoxy)-3,6,9,12-tetraoxapentadecyl)-1H-1,2,3-triazol-4-yl)methyl)tetrahydro-2H-pyran-2-yl)ethyl)phosphonic acid (I-101B). Yield: 0.101 g, 44.1%, LCMS, m/z 738.20 [M+1]+; ¹H NMR (400 MHz, DMSO-d₆ with D₂O exchange) δ 4.44 (t, J=5.2 Hz, 2H), 3.89-3.86 (m, 1H), 3.77-3.73 (m, 4H), 3.60-3.56 (m, 2H), 3.53-3.46 (m, 13H), 3.29-3.28 (m, 2H), 2.97 (t, J=5.6 Hz, 2H), 2.86 (d, J=7.2 Hz, 2H), 1.82 (bs, 1H), 1.57 (bs, 1H), 1.46-1.31 (m, 2H).

### Example 102: Synthesis of Compound I-102 (See Above)

### Example 103: Synthesis of Compound I-103

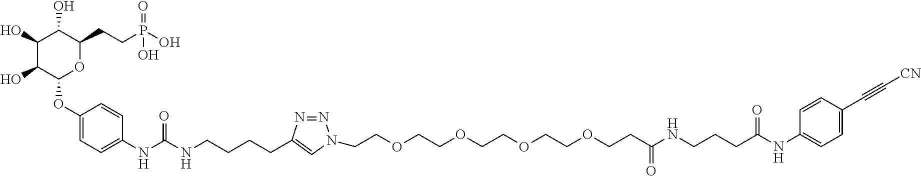 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1]

Compound I-103 was prepared using similar methods. Yield: 19 mg, 48%. H NMR (300 MHz, DMSO) δ 10.36 (s, 1H), 8.25 (s, 1H), 7.90 (d, J=5.5 Hz, 1H), 7.82 (s, 1H), 7.74 (s, 4H), 7.29 (d, J=9.0 Hz, 2H), 6.91 (d, J=9.0 Hz, 2H), 6.07 (s, 1H), 5.26 (d, J=1.8 Hz, 1H), 4.46 (t, J=5.2 Hz, 2H), 3.95-3.75 (m, 2H), 3.75-3.55 (m, 87H), 3.49 (d, J=2.3 Hz, 1H), 3.32 (d, J=6.7 Hz, 2H), 3.08 (t, J=6.0 Hz, 4H), 2.63 (t, J=7.4 Hz, 2H), 2.33 (dt, J=17.6, 6.9 Hz, 4H), 1.71 (p, J=6.9 Hz, 2H), 1.65-1.55 (m, 1H), 1.47 (d, J=7.8 Hz, 2H). LC-MS m/z 974 [M+1]⁺.

### Example 104: Synthesis of Compound I-104

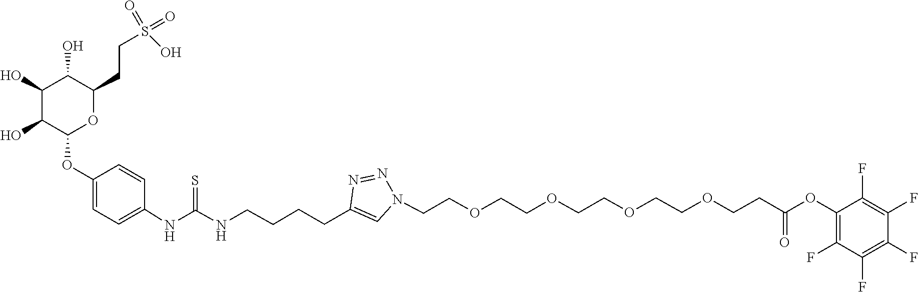 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F]

Compound 1-104 was prepared using similar methods. Yield: 9.5 mg, 42% yield. ¹H NMR (300 MHz, DMSO) δ 9.28 (s, 1H), 7.84 (s, 1H), 7.58 (s, 1H), 7.24 (d, J=8.3 Hz, 2H), 6.97 (d, J=8.4 Hz, 2H), 5.31 (s, 1H), 4.46 (t, J=5.2 Hz, 2H), 3.77 (q, J=6.2, 5.7 Hz, 6H), 3.55-3.40 (m, 16H), 3.33 (q, J=7.8, 6.1 Hz, 2H), 3.02 (t, J=5.9 Hz, 2H), 2.62 (d, J=7.0 Hz, 2H), 2.10 (q, J=14.4, 14.0 Hz, 2H), 1.58 (s, 5H). LC-MS m/z 947 [M+1]⁺.

### Example 105. Synthesis of Compound I-105 (Cpd. No. 567)

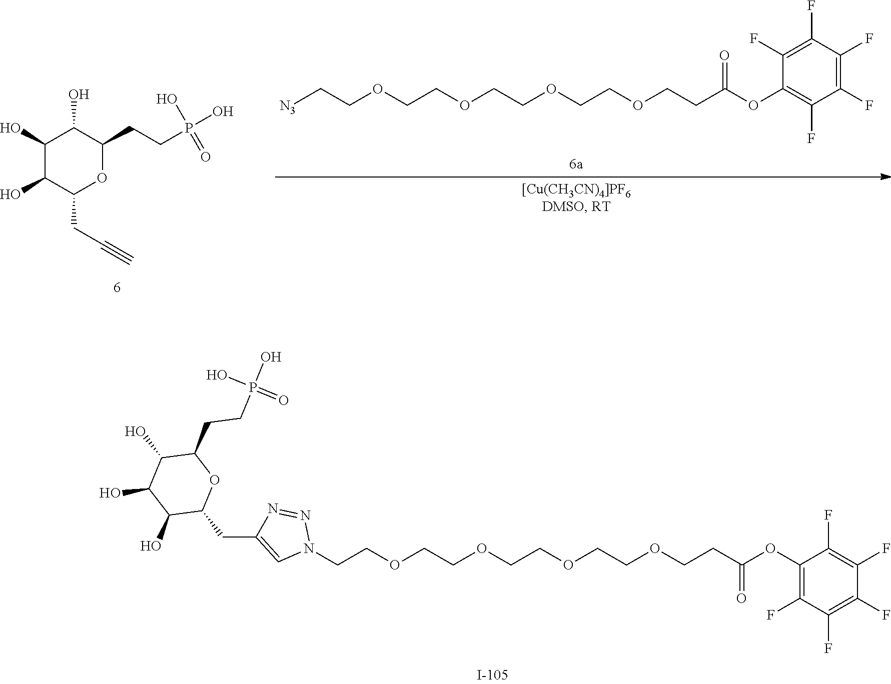

Compound I-105 is prepared from (2-((2R,3S,4R,5S,6R)-3,4,5-trihydroxy-6-(prop-2-yn-1-yl)tetrahydro-2H-pyran-2 yl)ethyl)phosphonic acid (6) using similar methods. Yield: 0.101 g, 44.11%, LCMS, m/z 738.20 [M+1]+; H NMR (400 MHz, DMSO-d₆ with D₂O exchange) δ 4.44 (t, J=5.2 Hz, 2H), 3.89-3.86 (m, 1H), 3.77-3.73 (m, 4H), 3.60-3.56 (m, 2H), 3.53-3.46 (m, 13H), 3.29-3.28 (m, 2H), 2.97 (t, J=5.6 Hz, 2H), 2.86 (d, J=7.2 Hz, 2H), 1.82 (bs, 1H), 1.57 (bs, 1H), 1.46-1.31 (m, 2H).

### Example 106: Synthesis of Compound I-106

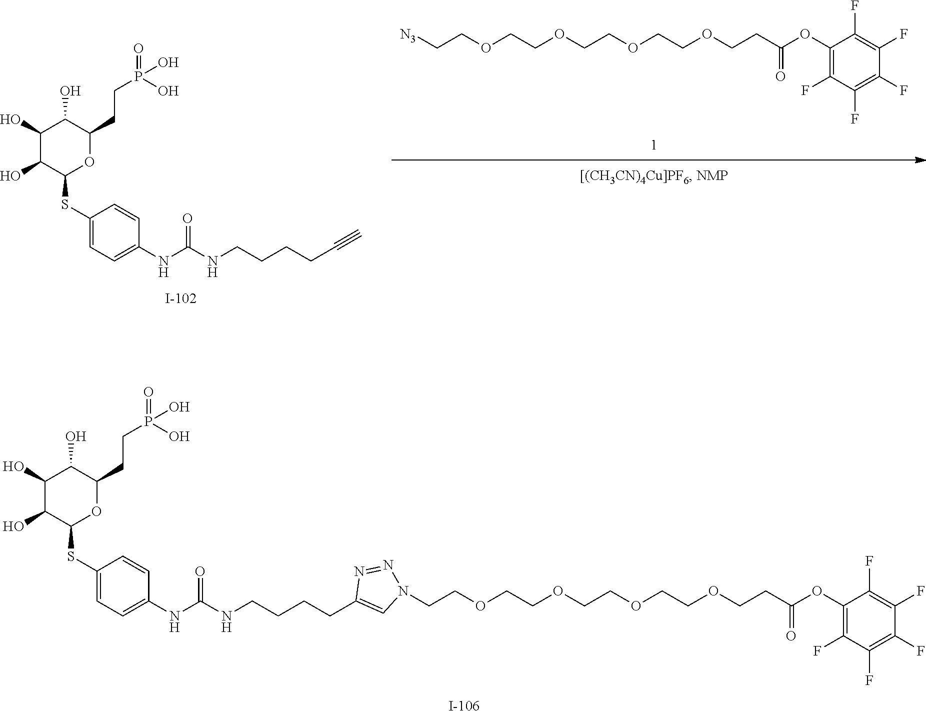 [SMILES: C#CCCCCNC(=O)Nc1ccc(S[C@@H]2O[C@H](CCP(=O)(O)O)[C@@H](O)[C@H](O)[C@@H]2O)cc1 ; [N-]=[N+]=NCCOCCOCCOCCOCCC(=O)Oc1c(F)c(F)c(F)c(F)c1F ; O=C(NCCCCc1cn(CCOCCOCCOCCOCCC(=O)Oc2c(F)c(F)c(F)c(F)c2F)nn1)Nc1ccc(S[C@@H]2O[C@H](CCP(=O)(O)O)[C@@H](O)[C@H](O)[C@@H]2O)cc1 ; "I-106"]

Compound I-106 is prepared from I-102 using similar methods. Yield: 17.4 mg, 64%; LCMS m/z 946.5 [M+1]+; ¹H NMR (300 MHz, DMSO-d₆ with D₂O) δ 7.76 (s, 1H), 7.33-7.19 (m, 4H), 4.73 (s, 1H), 4.40 (bs, 2H), 3.81 (bs, 1H), 3.78-3.65 (m, 4H), 3.56-2.84 (m, 19H), 2.63-2.54 (m, 2H), 2.03-1.89 (m, 1H), 1.81-1.29 (m, 7H).

### Example 107: Synthesis of Compound I-107

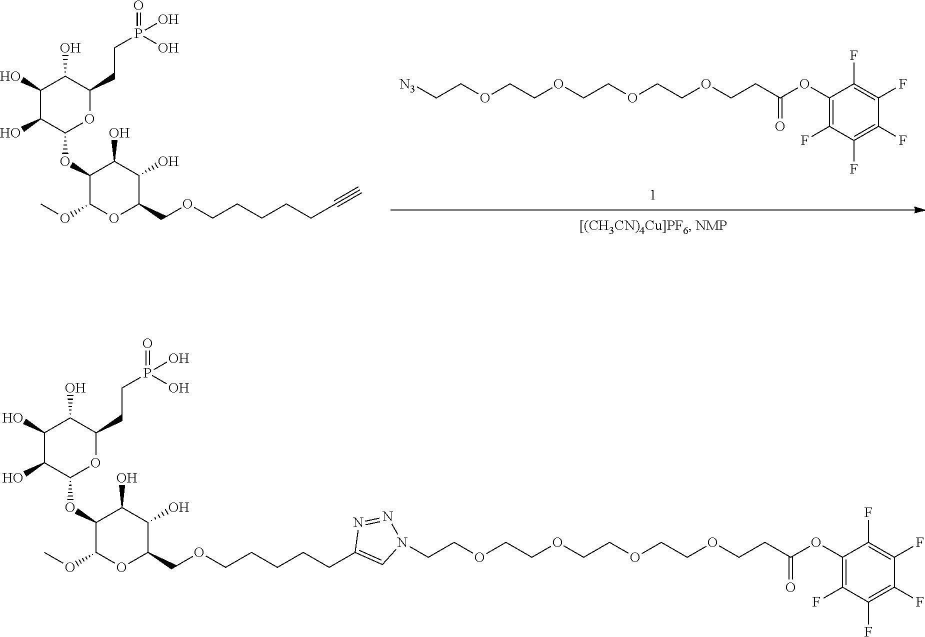 [SMILES: C#CCCCCCOC[C@H]1O[C@H](OC)[C@@H](O[C@H]2O[C@H](CCP(=O)(O)O)[C@@H](O)[C@H](O)[C@@H]2O)[C@@H](O)[C@@H]1O ; [N-]=[N+]=NCCOCCOCCOCCOCCC(=O)Oc1c(F)c(F)c(F)c(F)c1F ; CO[C@H]1O[C@H](COCCCCCc2cn(CCOCCOCCOCCOCCC(=O)Oc3c(F)c(F)c(F)c(F)c3F)nn2)[C@@H](O)[C@H](O)[C@@H]1O[C@H]1O[C@H](CCP(=O)(O)O)[C@@H](O)[C@H](O)[C@@H]1O]

Compound I-107 is prepared from I-102 using similar methods. Yield: 25.3 mg, 52%; LCMS m/z 986.6 [M+1]+; ¹H NMR (300 MHz, DMSO-d₆ with D₂O) δ 7.75 (s, 1H), 4.84 (s, 1H), 4.54 (s, 1H), 4.41 (t, J=5.1 Hz, 2H), 3.92-3.17 (m, 31H), 2.95 (t, J=5.8 Hz, 2H), 2.56 (t, J=7.6 Hz, 2H), 2.03-1.84 (m, 1H), 1.83-1.62 (m, 1H), 1.62-1.36 (m, 6H), 1.36-1.22 (m, 2H).

### Examples 108-109: Synthesis of Compounds I-108 to I-109 (see above)

### Example 110: Synthesis of Compound I-110

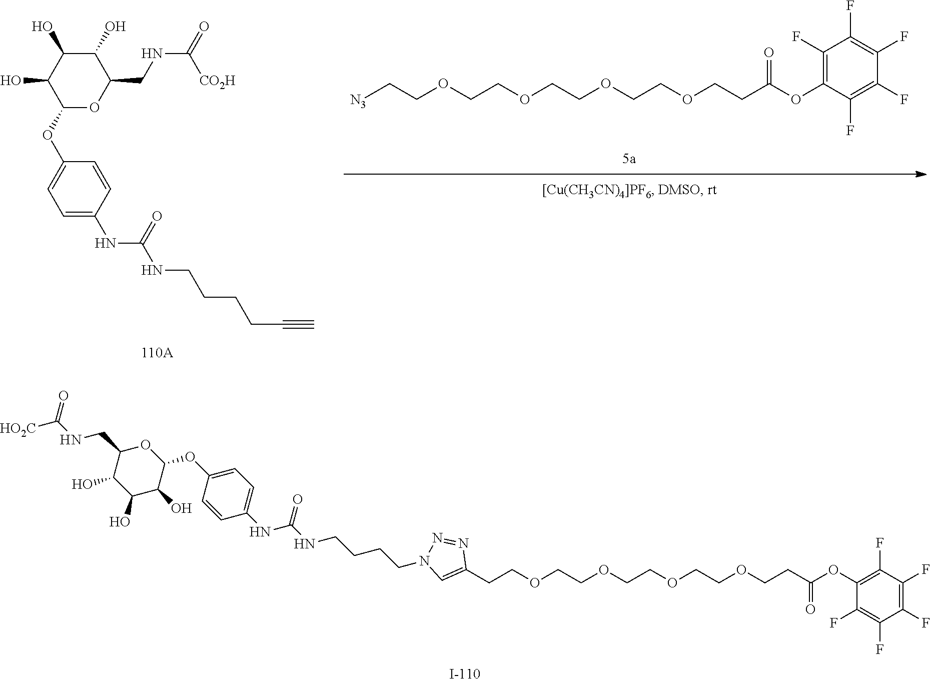 [SMILES: C#CCCCCNC(=O)Nc1ccc(O[C@H]2O[C@H](CNC(=O)C(=O)O)[C@@H](O)[C@H](O)[C@@H]2O)cc1 ; [N-]=[N+]=NCCOCCOCCOCCOCCC(=O)Oc1c(F)c(F)c(F)c(F)c1F ; O=C(NCCCCn1cc(CCOCCOCCOCCOCCC(=O)Oc2c(F)c(F)c(F)c(F)c2F)nn1)Nc1ccc(O[C@H]2O[C@H](CNC(=O)C(=O)O)[C@@H](O)[C@H](O)[C@@H]2O)cc1 ; "I-110"]

Compound I-110 was prepared from 110A using similar methods. Yield: 0.018 g, 50.23%; LCMS m/z 923.5 [M+1]+; ¹H NMR (400 MHz, DMSO-d₆ with D₂O) δ 7.78 (s, 1H), 7.17 (d, J=8.8 Hz, 2H), 6.87 (d, J=8.8 Hz, 2H), 5.12 (s, 1H), 4.43-4.40 (m, 2H), 3.76-3.71 (m, 4H), 3.63-3.56 (m, 2H), 3.49-3.37 (m, 15H), 3.35-3.21 (m, 1H), 3.04 (t, J=6.8 Hz, 2H), 2.94 (t, J=6.0 Hz, 2H), 2.59 (t, J=7.2 Hz, 2H), 2.10-2.04 (m, 1H), 1.58-1.55 (m, 2H), 1.43-1.40 (m, 2H).

### Example 111: Synthesis of Compound I-111

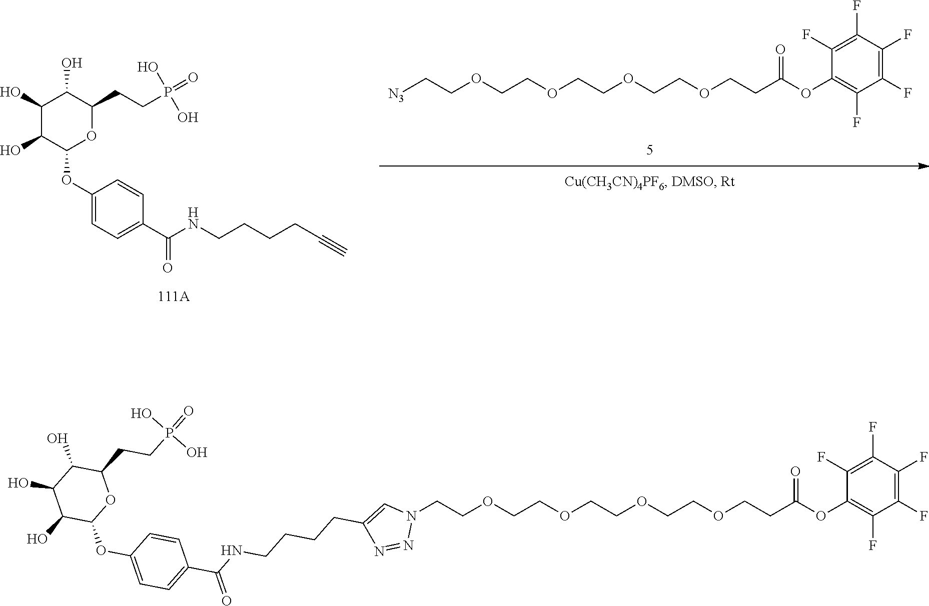 [SMILES: C#CCCCCNC(=O)c1ccc(O[C@H]2O[C@H](CCP(=O)(O)O)[C@@H](O)[C@H](O)[C@@H]2O)cc1 ; [N-]=[N+]=NCCOCCOCCOCCOCCC(=O)Oc1c(F)c(F)c(F)c(F)c1F ; O=C(CCOCCOCCOCCOCCn1cc(CCCCNC(=O)c2ccc(O[C@H]3O[C@H](CCP(=O)(O)O)[C@@H](O)[C@H](O)[C@@H]3O)cc2)nn1)Oc1c(F)c(F)c(F)c(F)c1F]

Compound I-111 was prepared from 111A using similar methods. Yield: 0.038 g, 47.50%; LCMS, m/z 915.42 [M+1]+; H NMR (400 MHz, DMSO-d₆) δ 8.35 (t, J=5.2 Hz, 1H), 7.81-7.79 (m, 3H), 7.08 (d, J=8.8 Hz, 2H), 5.47 (s, 1H), 4.48 (t, J=5.2 Hz, 2H), 3.82-3.74 (m, 5H), 3.65-3.62 (m, 1H), 3.55-3.46 (m, 12H), 3.34-3.22 (m, 4H), 3.01 (t, J=6.0 Hz, 2H), 2.67-2.61 (m, 2H), 2.00-1.92 (m, 1H), 1.62-1.54 (m, 5H), 1.20-1.05 (m, 1H).

### Example 112: Synthesis of Compound I-112

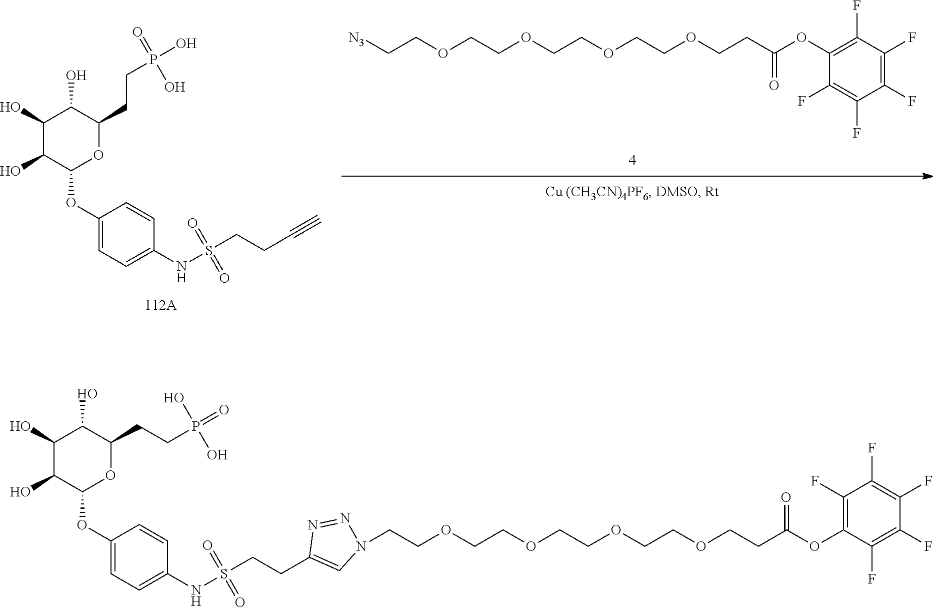 [SMILES: C#CCCS(=O)(=O)Nc1ccc(O[C@H]2O[C@H](CCP(=O)(O)O)[C@@H](O)[C@H](O)[C@@H]2O)cc1 ; [N-]=[N+]=NCCOCCOCCOCCOCCC(=O)Oc1c(F)c(F)c(F)c(F)c1F ; O=C(CCOCCOCCOCCOCCn1cc(CCS(=O)(=O)Nc2ccc(O[C@H]3O[C@H](CCP(=O)(O)O)[C@@H](O)[C@H](O)[C@@H]3O)cc2)nn1)Oc1c(F)c(F)c(F)c(F)c1F]

Compound I-112 was prepared from 112A using similar methods. Yield: 0.025 g; 50.44%. LCMS: m/z 923.44 [M+1]+; ¹H NMR (400 MHz, DMSO-d₆ with D₂O) δ 7.82 (d, J=4.8 Hz, 1H), 7.14-7.11 (m, 2H), 7.02-6.97 (m, 2H), 5.30 (s, 1H), 4.40 (t, J=4.8 Hz, 2H), 3.80-3.70 (m, 4H), 3.69-3.49 (m, 2H), 3.48-3.38 (m, 13H), 3.32-3.26 (m, 4H), 3.02-3.00 (m, 2H), 2.93-2.90 (m, 1H), 1.89 (bs, 2H), 1.69-1.39 (m, 2H), 1.25-1.10 (m, 1H).

### Example 113: Synthesis of Compound I-113

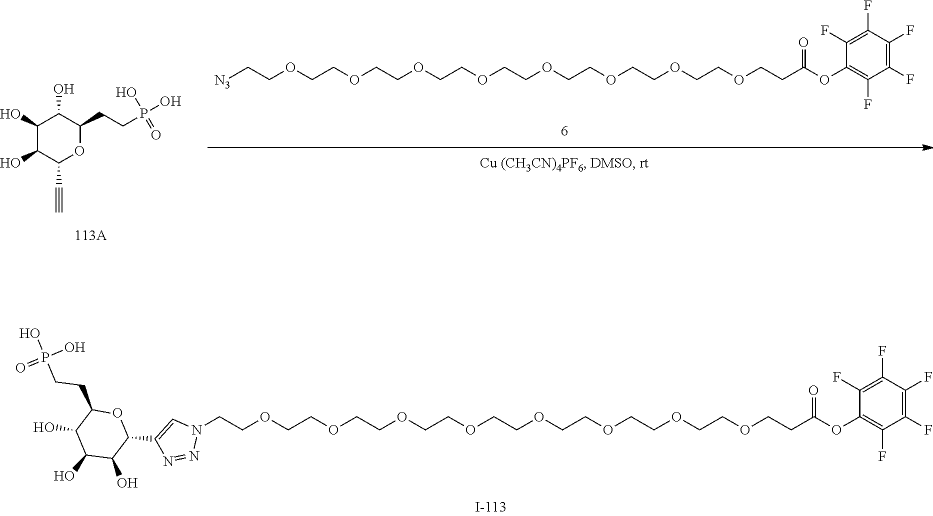 [SMILES: C#C[C@H]1O[C@H](CCP(=O)(O)O)[C@@H](O)[C@H](O)[C@@H]1O ; [N-]=[N+]=NCCOCCOCCOCCOCCOCCOCCOCCOCCC(=O)Oc1c(F)c(F)c(F)c(F)c1F ; O=C(CCOCCOCCOCCOCCOCCOCCOCCOCCn1cc([C@H]2O[C@H](CCP(=O)(O)O)[C@@H](O)[C@H](O)[C@@H]2O)nn1)Oc1c(F)c(F)c(F)c(F)c1F]

Compound I-113 was prepared from 113A using similar methods. Yield: 0.028 g, 25.89%; LCMS, m/z 900.47 [M+1]+; ¹H NMR (400 MHz, DMSO-d₆ with D₂O exchange) δ 7.97 (s, 1H), 4.88 (d, J=2.0 Hz, 1H), 4.49 (t, J=4.4 Hz, 2H), 4.21 (t, J=2.8 Hz, 1H), 3.80-3.78 (m, 2H), 3.50-3.45 (m, 30H), 3.11 (t, J=8.8 Hz, 1H), 2.97 (t, J=6.0 Hz, 2H), 2.42 (t, J=6.0 Hz, 2H), 1.89 (bs, 1H), 1.78-1.71 (m, 1H), 1.67-1.61 (m, 1H), 1.48-1.41 (m, 1H).

### Examples 114-118B. Additional Compounds

The following compounds were synthesized from synthon M6PR binding moieties and linker intermediates adapting the materials and methods described above.

| Com- | | LCMS |
|---|---|---|
| pound | | m/z |
| # | Structure | observed |
| I-114 | 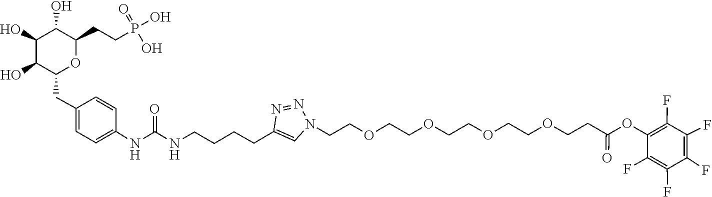 [SMILES: O=C(NCCCCc1cn(CCOCCOCCOCCOCCC(=O)Oc2c(F)c(F)c(F)c(F)c2F)nn1)Nc1ccc(C[C@H]2O[C@H](CCP(=O)(O)O)[C@@H](O)[C@H](O)[C@@H]2O)cc1] | 928.6 [M + 1]⁺ |
| I-115 | 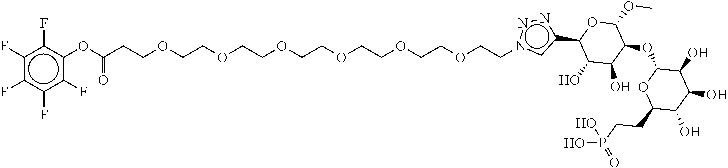 | 974.5 [M + 1]+ |
| I-116 | 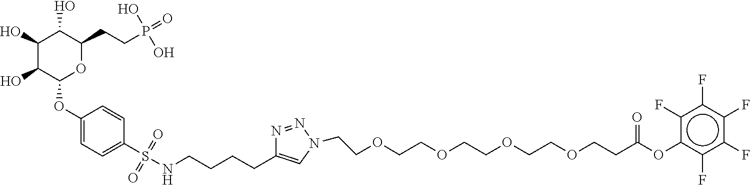 | 951.4 [M + 1]+ |
| I-117 | 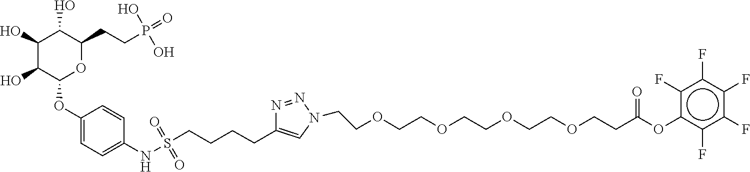 | 951.4 [M + 1]+ |
| I-118 | 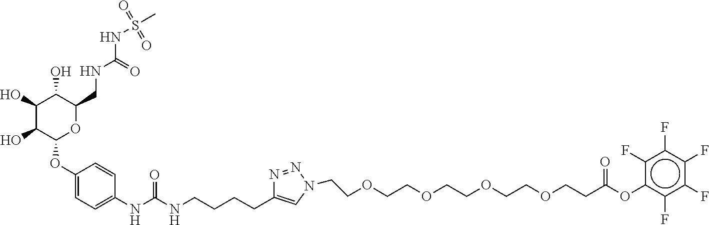 | 972.5 [M + 1]+ |
| I-118A | 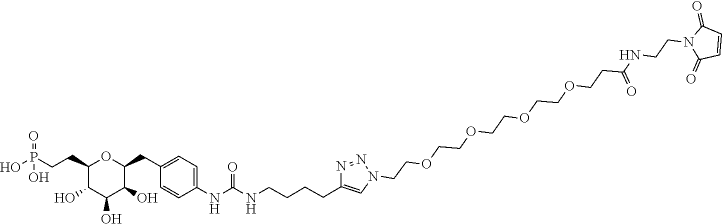 [SMILES: O=C(CCOCCOCCOCCOCCn1cc(CCCCNC(=O)Nc2ccc(C[C@@H]3O[C@H](CCP(=O)(O)O)[C@@H](O)[C@H](O)[C@@H]3O)cc2)nn1)NCCN1C(=O)C=CC1=O] | 844.2 [M + H]+ |
| I-118B | 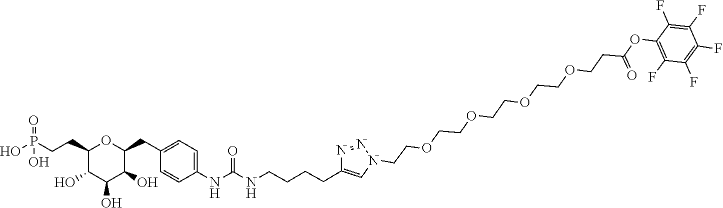 [SMILES: O=C(NCCCCc1cn(CCOCCOCCOCCOCCC(=O)Oc2c(F)c(F)c(F)c(F)c2F)nn1)Nc1ccc(C[C@@H]2O[C@H](CCP(=O)(O)O)[C@@H](O)[C@H](O)[C@@H]2O)cc1] | 928.1 [M + H]+ |

### Example 118C-118E. Additional Multivalent Linkers

Synthesis of (4S,7S,26S,29S)-4,7,26,29-tetrakis(4-azidobutyl)-2,5,8,24,27,30-hexaoxo-12,15,18,21,34,37,40,43-octaoxa-3,6,9,25,28,31-hexaazahexatetracontan-46-oic acid (I-118C).

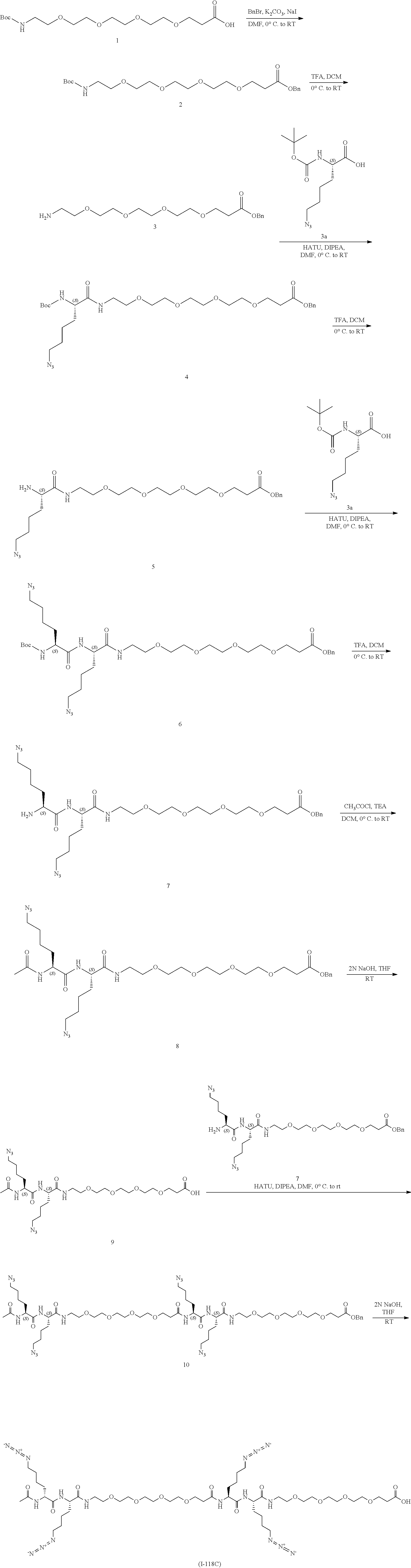

To a solution of 2,2-dimethyl-4-oxo-3,8,11,14,17-pentaoxa-5-azaicosan-20-oic acid (1, 1.0 eq, 5.0 g, 13.7 mmol) in N,N-dimethylformamide (50 mL), potassium carbonate (3.0 eq, 5.67 g, 41.0 mmol) and sodium iodide (0.1 eq, 0.205 g, 1.37 mmol) were added, cooled at 0° C., benzyl bromide (1.5 eq, 2.51 mL, 20.5 mmol) was added and reaction mixture was stirred at room temperature for 16 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-3% methanol in dichloromethane to afford benzyl 2,2-dimethyl-4-oxo-3,8,11,14,17-pentaoxa-5-azaicosan-20-oate (2) as a colourless viscous liquid. Yield: 5.6 g, 89.65%; LCMS m/z 456.25 [M+1]⁺.

A solution of benzyl 2,2-dimethyl-4-oxo-3,8,11,14,17-pentaoxa-5-azaicosan-20-oate (2, 1.0 eq, 2.7 g, 5.93 mmol) in dichloromethane (13.5 mL) was cooled at 0° C., trifluoroacetic acid (13.5 mL) was added and reaction mixture was stirred at room temperature for 1 h. After completion, reaction mixture was concentrated, azeotroped with dichloromethane (2-3 times) and dried to afford benzyl 1-amino-3,6,9,12-tetraoxapentadecan-15-oate (3) as a light brown viscous liquid. Yield: 4.2 g (Crude); LCMS m/z 356.20 [M+1]⁺.

A solution of N2-(tert-butoxycarbonyl)-N6-diazo-L-lysine (3a, 1.0 eq, 1.6 g, 5.88 mmol) and benzyl 1-amino-3,6,9,12-tetraoxapentadecan-15-oate (3, 2.0 eq, 4.18 g, 11.8 mmol) in N,N-dimethylformamide (26 mL) was cooled at 0° C., N,N diisopropylethylamine (5.0 eq, 5.12 mL, 29.4 mmol) and 1-[bis(dimethylamino)methylene]-1H-1,2,3-triazolo[4,5-b]pyridinium 3-oxid hexafluorophosphate (HATU) (1.5 eq, 3.35 g, 8.81 mmol) were added and reaction mixture was stirred at room temperature for 3 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-10% methanol in dichloromethane to afford benzyl (S)-6-(4-azidobutyl)-2,2-dimethyl-4,7-dioxo-3,11,14,17,20-pentaoxa-5,8-diazatricosan-23-oate (4) as a light brown viscous liquid. Yield: 3.0 g, 83.74%; LCMS m/z 610.10 [M+1]⁺.

A solution of benzyl (S)-6-(4-azidobutyl)-2,2-dimethyl-4,7-dioxo-3,11,14,17,20-pentaoxa-5,8-diazatricosan-23-oate (4, 1.0 eq, 3.3 g, 5.41 mmol) in dichloromethane (16.5 mL) was cooled at 0° C., trifluoroacetic acid (16.5 mL) was added and reaction mixture was stirred at 0° C. for 1 h. After completion, reaction mixture was concentrated, azeotroped with dichloromethane (2-3 times), washed with diethyl ether and dried to afford benzyl (S)-18-amino-22-azido-17-oxo-4,7,10,13-tetraoxa-16-azadocosanoate (5) as a light brown viscous liquid. Yield: 1.9 g (Crude); LCMS m/z 510.20 [M+1]⁺.

A solution of N2-(tert-butoxycarbonyl)-N6-diazo-L-lysine (3a, 1.0 eq, 0.850 g, 3.12 mmol) and benzyl (S)-18-amino-22-azido-17-oxo-4,7,10,13-tetraoxa-16-azadocosanoate (5, 1.1 eq, 1.75 g, 3.43 mmol) in N,N-dimethylformamide (10 mL) was cooled at 0° C., N,N diisopropylethylamine (5.0 eq, 2.72 mL, 15.6 mmol) and 1-[bis(dimethylamino)methylene]-1H-1,2,3-triazolo[4,5-b]pyridinium 3-oxid hexafluorophosphate (HATU) (1.5 eq, 1.78 g, 4.68 mmol) were added and reaction mixture was stirred at room temperature for 3 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-6% methanol in dichloromethane to afford benzyl (6S,9S)-6,9-bis(4-azidobutyl)-2,2-dimethyl-4,7,10-trioxo-3,14,17,20,23-pentaoxa-5,8,11-triazahexacosan-26-oate (6) as a colourless viscous liquid. Yield: 1.68 g, 68.24%; LCMS m/z 764.10 [M+1]⁺.

A solution of benzyl (6S,9S)-6,9-bis(4-azidobutyl)-2,2-dimethyl-4,7,10-trioxo-3,14,17,20,23-pentaoxa-5,8,11-triazahexacosan-26-oate (6, 1.0 eq, 1.68 g, 2.2 mmol) in dichloromethane (8.5 mL) was cooled at 0° C., trifluoroacetic acid (8.5 mL) was added and reaction mixture was stirred at 0° C. for 1 h. After completion, reaction mixture was concentrated, azeotroped with dichloromethane (2-3 times), washed with diethyl ether and dried to afford benzyl (S)-18-amino-22-azido-17-oxo-4,7,10,13-tetraoxa-16-azadocosanoate (7) as a light brown viscous liquid. Yield: 1.6 g (Crude); LCMS m/z 664.20 [M+1]⁺.

A solution of benzyl (18S,21S)-21-amino-25-azido-18-(4-azidobutyl)-17,20-dioxo-4,7,10,13-tetraoxa-16,19-diazapentacosanoate (7, 1.0 eq, 0.600 g, 0.904 mmol) in dichloromethane (6 mL) was cooled at 0° C., triethylamine (3.0 eq, 0.39 mL, 2.71 mmol) and acetyl chloride (1.5 eq, 0.096 mL, 1.36 mmol) were added and reaction mixture was stirred at room temperature for 2 h. Reaction was monitored by LCMS. After completion, reaction mixture was concentrated to get crude which was purified by column chromatography using silica gel (100-200 mesh) and 0-5% methanol in dichloromethane to afford benzyl (4S,7S)-4,7-bis(4-azidobutyl)-2,5,8-trioxo-12,15,18,21-tetraoxa-3,6,9-triazatetracosan-24-oate (8) as a light yellow viscous liquid. Yield: 0.390 g, 60.98%; LCMS m/z 706.35 [M+1]⁺.

To a solution of benzyl (4S,7S)-4,7-bis(4-azidobutyl)-2,5,8-trioxo-12,15,18,21-tetraoxa-3,6,9-triazatetracosan-24-oate (8, 1.0 eq, 0.390 g, 0.553 mmol) in tetrahydrofuran (4 mL), 2N aqueous sodium hydroxide solution (2.0 eq, 0.55 mL, 1.11 mmol) was added and reaction mixture was stirred at room temperature for 16 h. After completion, reaction mixture was concentrated and purified by prep HPLC (23-35% acetonitrile in water with 0.05% trifluoroacetic acid). Fractions containing the desired product were combined and lyophilized to dryness to afford (4S,7S)-4,7-bis(4-azidobutyl)-2,5,8-trioxo-12,15,18,21-tetraoxa-3,6,9-triazatetracosan-24-oic acid (9) as a colourless semi solid. Yield: 0.186 g, 54.7%; LCMS m/z 616.30 [M+1]+; ¹H NMR (400 MHz, DMSO-d₆ with D₂O) 64.16-4.13 (m, 2H), 3.57 (t, J=6.0 Hz, 2H), 3.47-3.46 (m, 1OH), 3.38 (t, J=5.6 Hz, 2H), 3.29-3.23 (m, 4H), 3.21-3.18 (m, 1H), 3.16-3.11 (m, 1H), 2.41 (t, J=6.4 Hz, 2H), 1.83 (s, 3H), 1.68-1.58 (m, 2H), 1.55-1.42 (m, 7H), 1.33-1.22 (m, 4H).

A solution of (4S,7S)-4,7-bis(4-azidobutyl)-2,5,8-trioxo-12,15,18,21-tetraoxa-3,6,9-triazatetracosan-24-oic acid (9, 1.0 eq, 0.700 g, 1.14 mmol) and benzyl (18S,21S)-21-amino-25-azido-18-(4-azidobutyl)-17,20-dioxo-4,7,10,13-tetraoxa-16,19-diazapentacosanoate (7, 0.8 eq, 0.604 g, 0.910 mmol) in N,N-dimethylformamide (10 mL) was cooled at 0° C., N,N diisopropylethylamine (5.0 eq, 0.99 mL, 5.68 mmol) and 1-[bis(dimethylamino)methylene]-1H-1,2,3-triazolo[4,5-b]pyridinium 3-oxid hexafluorophosphate (HATU) (1.5 eq, 0.648 g, 1.71 mmol) were added and reaction mixture was stirred at room temperature for 3 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-6% methanol in dichloromethane to afford benzyl (4S,7S,26S,29S)-4,7,26,29-tetrakis(4-azidobutyl)-2,5,8,24,27,30-hexaoxo-12,15,18,21,34,37,40,43-octaoxa-3,6,9,25,28,31 hexaazahexatetracontan-46-oate (2) as a colourless viscous liquid. Yield: 0.300 g, 20.92%; LCMS m/z 631.32 [M+1]⁺.

To a solution of benzyl (4S,7S,26S,29S)-4,7,26,29-tetrakis(4-azidobutyl)-2,5,8,24,27,30-hexaoxo-12,15,18,21,34,37,40,43-octaoxa-3,6,9,25,28,31 hexaazahexatetracontan-46-oate (2, 1.0 eq, 0.350 g, 0.277 mmol) in tetrahydrofuran (4 mL), 2N aqueous sodium hydroxide solution (2.0 eq, 0.27 mL, 0.554 mmol) was added and reaction mixture was stirred at room temperature for 16 h. After completion, reaction mixture was concentrated and purified by prep HPLC (22-45% acetonitrile in water with 0.05% trifluoroacetic acid). Fractions containing the desired product were combined and lyophilized to dryness to afford (4S,7S,26S,29S)-4,7,26,29-tetrakis(4-azidobutyl)-2,5,8,24,27,30-hexaoxo-12,15,18,21,34,37,40,43-octaoxa-3,6,9,25,28,31-hexaazahexatetracontan-46-oic acid (I-118C) as a colorless viscous liquid. Yield: 0.090 g, 27.95%; LCMS m/z 586.40 [M+1]+; ¹H NMR (400 MHz, DMSO-d₆ with D₂O) δ 4.20-4.13 (m, 4H), 3.57 (t, J=6.0 Hz, 4H), 3.47-3.46 (m, 24H), 3.37 (t, J=5.6 Hz, 4H), 3.28-3.19 (m, 1OH), 3.17-3.11 (m, 2H), 2.41 (t, J=6.4 Hz, 2H), 2.38-2.31 (m, 2H), 1.83 (s, 3H), 1.65-1.56 (m, 4H), 1.55-1.42 (m, 12H), 1.34-1.21 (m, 8H).

Compounds I-118D was made by adapting the procedure above for the synthesis of I-121.

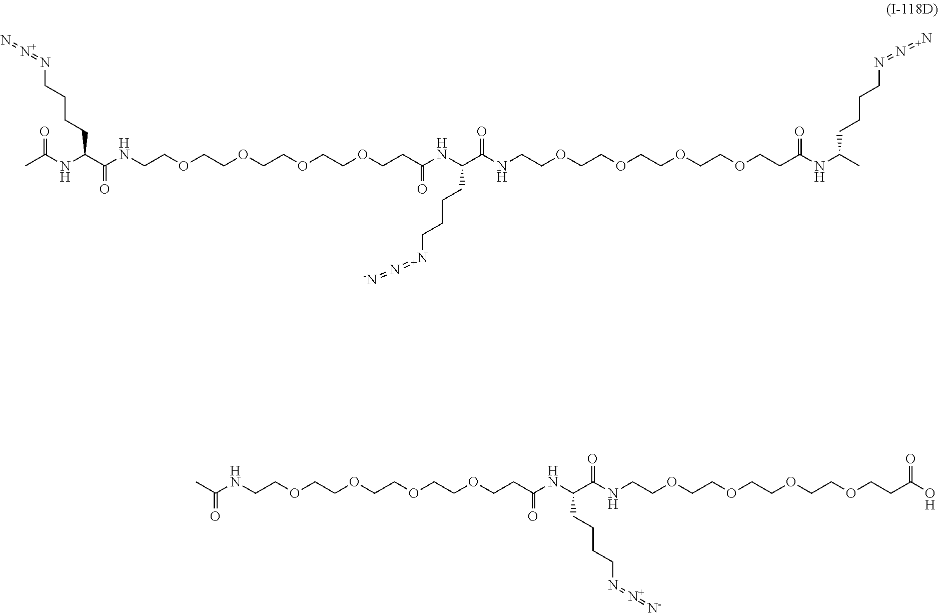

LCMS m/z 833.40 [(M/2)+1]*; ¹H NMR (400 MHz, DMSO-d6) δ 12.14 (bs, 1H), 7.97-7.94 (m, 8H), 4.23-4.21 (m, 4H), 3.61-3.56 (m, 8H), 3.49-3.48 (m, 45H), 3.40-3.37 (m, 8H), 3.32-3.27 (m, 8H), 3.23-3.15 (m, 1OH), 2.45-2.37 (m, 9H), 1.83 (s, 3H), 1.62-1.60 (m, 4H), 1.52-1.46 (m, 12H), 1.31-1.23 (m, 8H).

### Example 118F-118H

The following compounds were synthesized from synthon M6PR binding moieties and linker intermediates adapting the materials and methods described above.

Other Multivalent ligands

| | |
|---|---|
| I-118F | 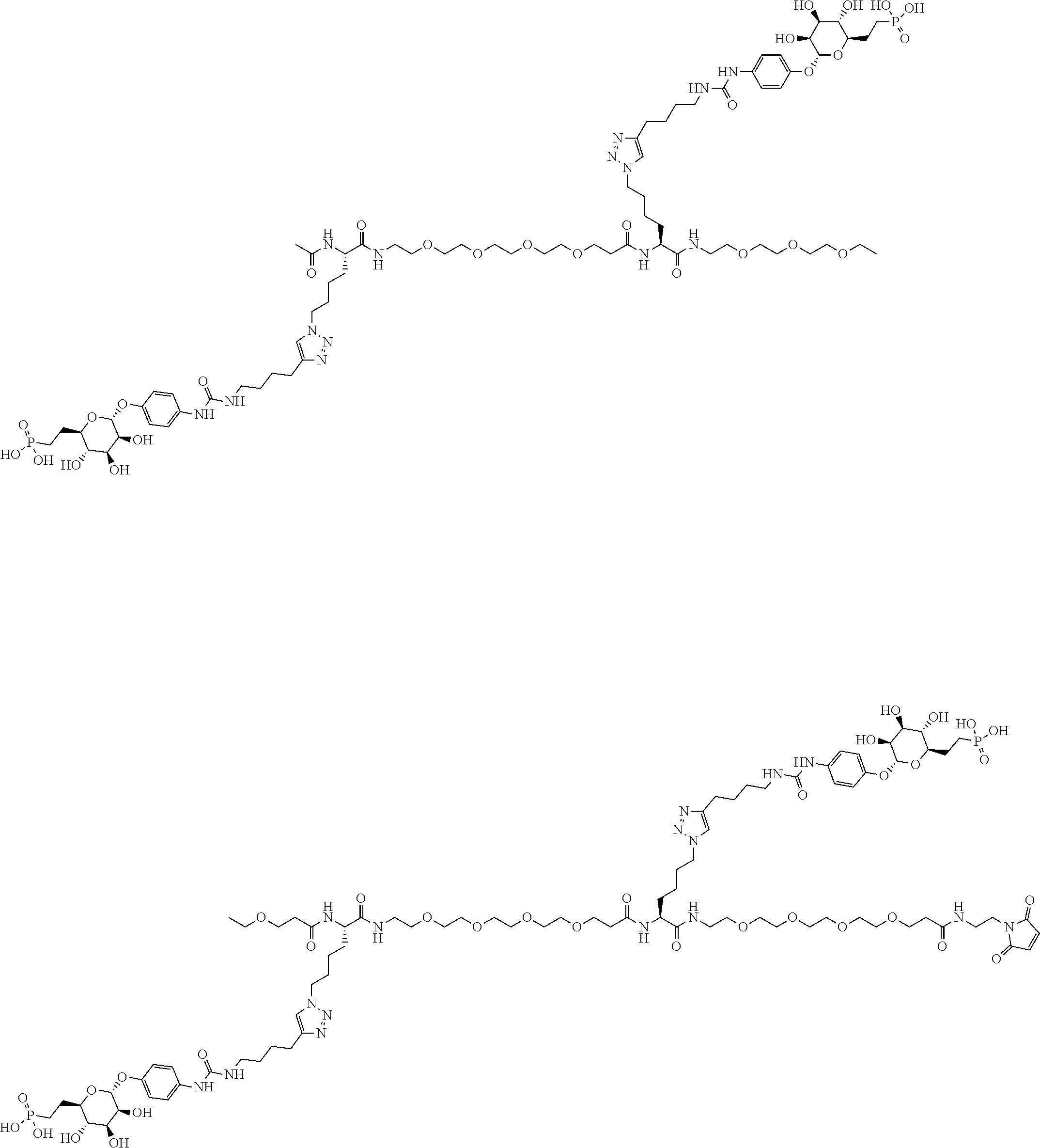 |
| | Synthesized by adapting the procedure for compound I-81 in example 81, using I-118D in place of intermediate 11 to provide compound I-118F. |
| | LCMS: 1838.8 [M/2 + H]++ |
| I-118G | 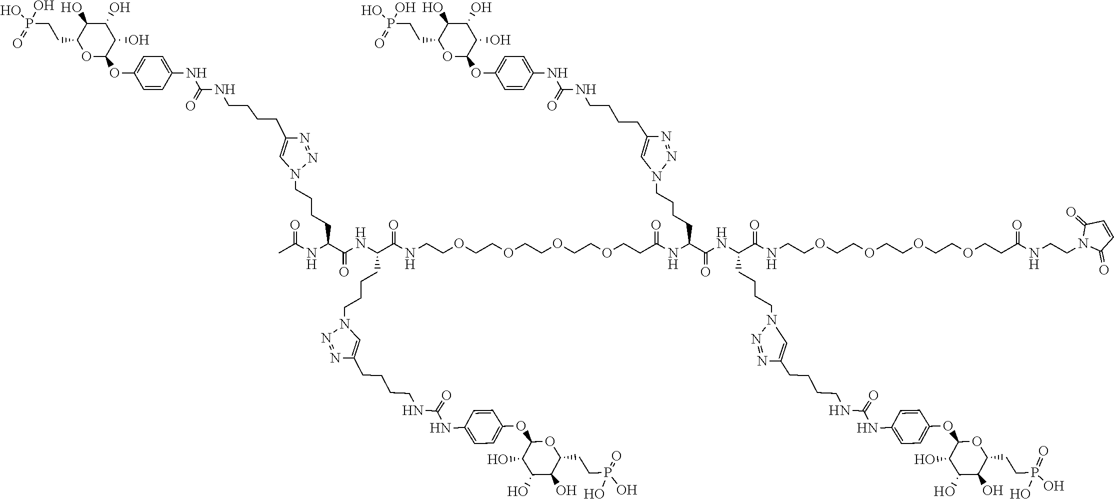 [SMILES: CC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| | Synthesized by adapting the procedure for compound I-81 in example 81, using I-118C in place of intermediate 11 to provide compound I-118G. |
| | LCMS: 1589.9 [M − 2H]− |

### 5.2. Preparation of Conjugates

### Example 119: Conjugation of Isothiocyanate-Based Ligand-Linker Compounds with Anti-EGFR and Anti-PD-L1 Antibodies

This example provides a general protocol for the conjugation of the isothiocyanate-based ligand-linker compounds (e.g., Compound A) with the primary amines on lysine residues of anti-EGFR antibodies (e.g., matuzumab, cetuximab) and anti-PD-L1 antibodies (e.g., atezolizumab, anti-PD-L1(29E.2A3)). The conjugates thus obtained are listed in Table 14.

The antibody was buffer exchanged into 100 mM sodium bicarbonate buffer pH 9.0 at 5 mg/mL concentration, after which about 30 equivalents of the isothiocyanate-based ligand-linker compound (e.g., Compound A; freshly prepared as 20 mM stock solution in DMSO) was added and incubated overnight at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

### Example 120: Conjugation of Perfluorophenoxy-Based Ligand-Linker Compounds with Anti-EGFR and IgG Antibodies

This example provides a general protocol for the conjugation of the perfluorophenoxy-based ligand-linker compounds (e.g., Compound I-7) with the primary amines on lysine residues of anti-EGFR antibodies (e.g., matuzumab, cetuximab) and IgG antibodies (e.g., IgG2a-UNLB). The conjugates thus obtained are listed in Table 14.

The antibody was buffer exchanged into 50 mM sodium phosphate buffer pH 8.0 at 5 mg/mL concentration, after which about 22 equivalents of perfluorophenoxy-based ligand-linker compound (e.g., Compound I-7; freshly prepared as 20 mM stock solution in DMSO) was added and incubated for 3 hours at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

### Example 121: Determination of DAR Values by Mass Spectrometry

This example provides the method for determining DAR values for the conjugates prepared as described in Examples 119 and 120. To determine the DAR value, 10 g of the antibody (unconjugated or conjugated) was treated 2 μL of non-reducing denaturing buffer (10×, New England Biolabs) for 10 minutes at 75° C. The denatured antibody solution was then deglycosylated by adding 1.5 μL of Rapid-PNGase F (New England Biolabs) and incubated for 10 minutes at 50° C. Deglycosylated samples were diluted 50-fold in water and analyzed on a Waters ACQUITY UPLC interfaced to Xevo G2—S QToF mass spectrometer. Deconvoluted masses were obtained using Waters MassLynx 4.2 Software. DAR values were calculated using a weighted average of the peak intensities corresponding to each loading species using the formula below:

$$DAR = \sum ( drug load distribution ( \% ) of each Ab with drug load n ) ( n ) / 100$$

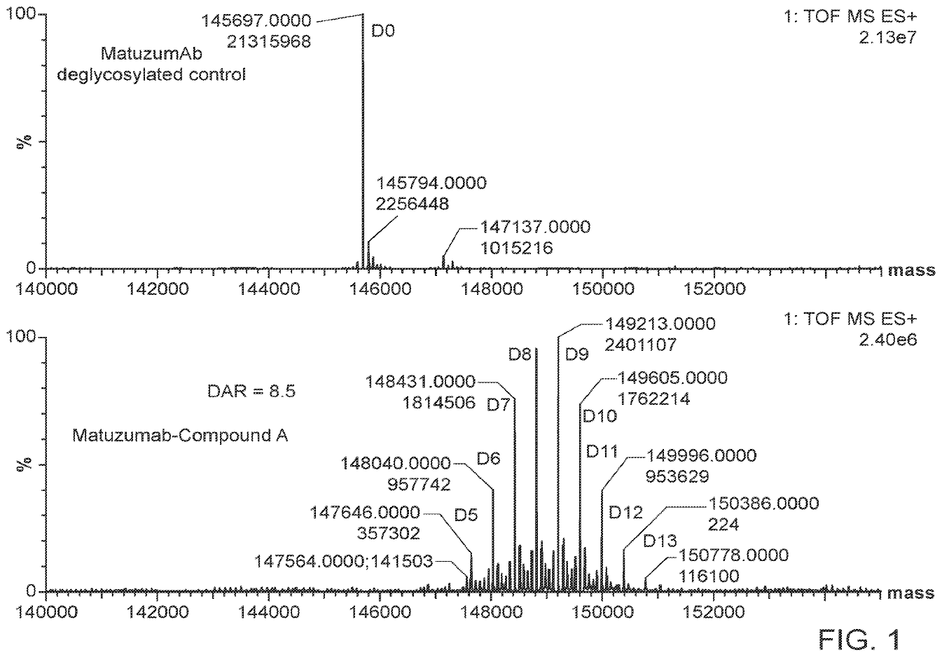

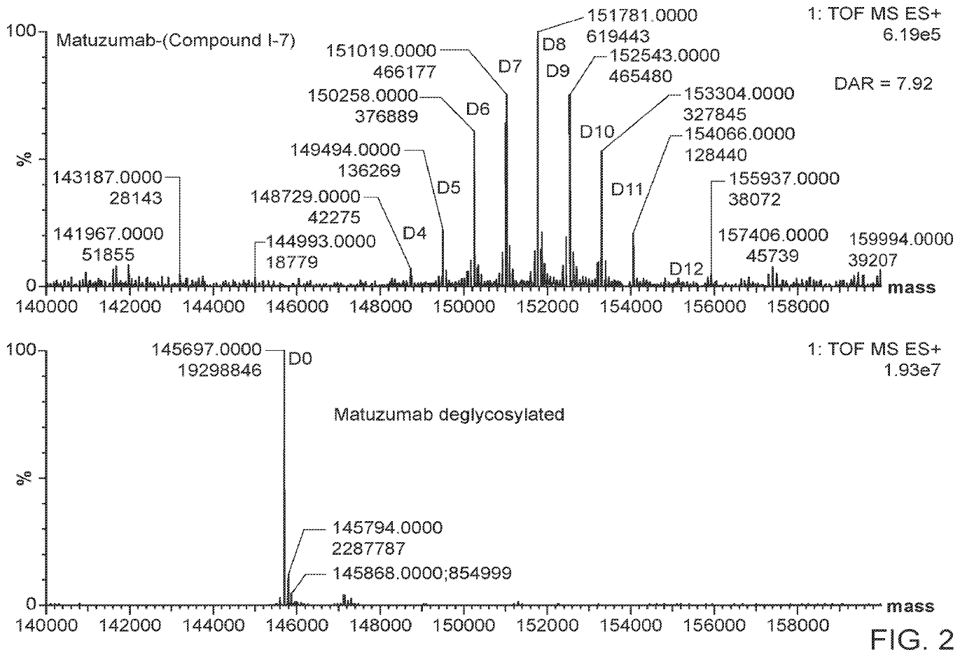

DAR values for the conjugates prepared as described in Examples 137 and 138 are shown in Table 14. Exemplary LC-MS data for one of the conjugates prepared as described in Examples 137 and 138 is shown in FIGS. 1-2.

### Example 122: Determination of Purity of Conjugates by SEC Method

Purity of the conjugates prepared as described in Examples 119 and 120 was determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL was loaded to a TSKgel SuperSW3000 column, at a constant flow rate of 0.35 mL/min. Chromatographs were integrated based on elution time to calculate the purity of monomeric conjugate species.

TABLE 14

| | | Ligand-Linker | | |
|---|---|---|---|---|
| Conjugate Name | Antibody | (Compd. No.) | DAR (by MS) | Purity (by SEC) |
| Matuzumab-(Compound A) | Matuzumab | Compound A | 8.5 | >98% |
| Matuzumab-(Compound 520 (I-7)) | Matuzumab | Compound I-7 | 7.9 | >98% |
| Atezolizumab-(Compound A) | Atezolizumab | Compound A | 12.1 | >96% |
| Cetuximab-(Compound A) | Cetuximab | Compound A | 7.8 | >97% |
| Cetuximab-(Compound 520 (I-7)) | Cetuximab | Compound I-7 | 7.72 | >98% |
| anti-PD-L1(29E.2A3)-(Compound A) | anti-PD- | Compound A | 7.9-8.5 | >96% |
| | L1(29E.2A3) | | | |
| anti-IgG2a-(Compound 520 (I-7)) | Anti-IgG2a | Compound I-7 | 7.9 | >99% |

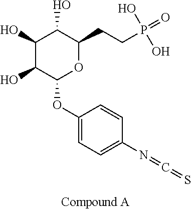 [SMILES: O=P(O)(O)CC[C@H]1O[C@H](Oc2ccc(N=C=S)cc2)[C@@H](O)[C@@H](O)[C@@H]1O]

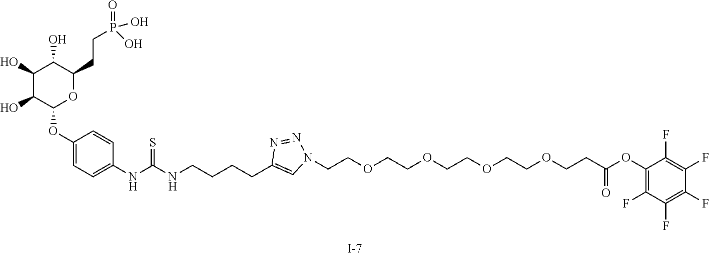

### Example 123: Antibody Disulfide Reduction and Thiol-Reactive Ligand-Linker Conjugation to Antibody

This example provides an exemplary protocol for reduction of the disulfides of the antibodies described herein, and conjugation of the reduced antibodies to thiol-reactive ligand-linker compounds described herein, e.g., containing a maleimide chemoselective ligation group.

Protocol: Antibody disulfide reduction
A) Dilute antibody to 15 mg/mL (0.1 mM IgG) in PBS, pH 7.4.
B) Prepare a fresh 20 mM (5.7 mg/mL) stock solution of tris(2 carboxyethyl)phosphine (TCEP) in H₂O.
C) Add 25 μL of TCEP stock solution from step B) above to 1 mL of antibody from step A) above (0.5 mM final concentration TCEP).
D) Incubate at 37° C. for 2 hours (check for free thiols using 5,5′-dithiobis-(2-nitrobenzoic acid) (DTNB) test).
E) Aliquot the reduced antibody into 4 tubes (250 μL each).
   Ligand-linker conjugation to antibody
A) Prepare 10 mM stock solution of thiol-reactive ligand-linker compound in DMSO (DMA, DMF or CH₃CN are also acceptable).
B) Add 5 equivalents of 12.5 μL stock solution from step A) above to each tube of reduced antibody (0.5 mM final concentration ligand-linker compound stock solution).
C) Incubate overnight at 4° C. for 4 hours at room temperature; check for free thiols using DTNB test.
D) Run analytical hydrophobic interaction chromatography (HIC) to determine DAR and homogeneity.

### Example 124: Preparation of Omalizumab Conjugates

A series of conjugates of the exemplary antibody omalizumab (an anti-IgE antibody) with a series of perfluorophenyl ester containing ligand-linker compounds were prepared and characterized using methods similar to those described in Examples 120-122.

These conjugates were assessed for cell uptake activity in two batches (set 1 and set 2) as described in Example 127.

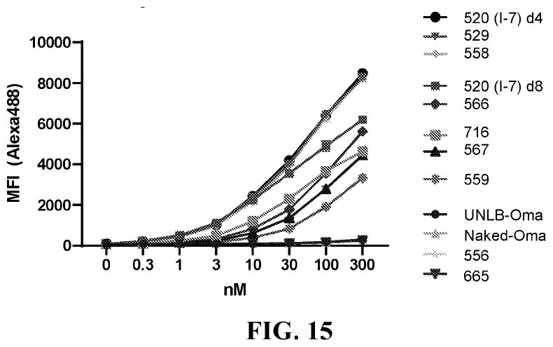

TABLE 15A
Omalizumab Conjugate Set 1 (see FIG. 15)

| Ligand-Linker | Chemoselective | DAR | Purity |
|---|---|---|---|
| (Compd. No.) | ligation group | (by MS) | (by SEC) |
| 520 (1-7) | PFP ester | 4 | >95% |
| 520 (1-7) | PFP ester | 8 | |
| 529 (1-38) | PFP ester | 4 | >95% |
| 558 | PFP ester | 4 | >95% |
| 566 | PFP ester | 4 | >95% |
| 716 | PFP ester | 4 | >95% |
| 567 | PFP ester | 4 | >95% |
| 559 | PFP ester | 4 | >95% |
| 556 (I-104) | PFP ester | 4 | >95% |
| 665 | PFP ester | 4 | >95% |

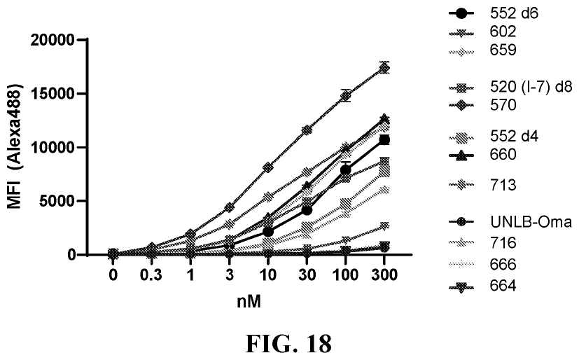

TABLE 15B
Omalizumab Conjugates (see FIG. 18)

| Ligand-Linker | Chemoselective | DAR | Purity |
|---|---|---|---|
| (Compd. No.) | ligation group | (by MS) | (by SEC) |
| 552 (1-95) | PFP ester | 6 | >95% |
| 602 (1-8) | PFP ester | 4 | >95% |
| 659 | PFP ester | 4 | >95% |
| 520 (1-7) | PFP ester | 8 | |
| 570 | PFP ester | 4 | >95% |
| 552 (1-95) | PFP ester | 4 | >95% |
| 660 | PFP ester | 4 | >95% |
| 713 | PFP ester | 4 | >95% |
| 716 (1-12) | PFP ester | 4 | >95% |
| 666 | PFP ester | 4 | >95% |
| 664 | PFP ester | 4 | >95% |

### 5.3. Assessment of Activity of Compounds and Conjugates

### Example 125: Reagents, Buffer, and Media

This example provides the reagents, buffer, and media used in the protocols described herein.

### Reagents:

Hela Cells (Sigma, #93021013)
Cetuximab (R&D systems)
Matuzumab (R&D systems)
Alexafluor647 labeling kit (Invitrogen)
Amicon filters, 30 kDa cut-off (Sigma Millipore)
DAPI (Invitrogen)
PFA (16% Paraformaldehyde Aqueous Solution, Electron Microscopy Sciences)
BSA (Bovine serum albumin; Sigma Millipore)
TrypLE (Invitrogen)
Accutase (Invitrogen)
Rabbit anti-EGFR (CST)
Mouse anti-β-actin (SCB)
Donkey anti-rabbit 800CW (Licor)
Donkey anti-mouse 680RD (Licor)
Odyssey Intercept Blocking Buffer (Licor)
Electroporation enhancer (IDT)
tracrRNA (IDT)
Amaxa Electroporator (Lonza)
SE Buffer (Lonza)
16-well electroporation cuvettes (Lonza)
M6P (D-Mannose-6 phosphate disodium salt hydrate; Sigma)
M6Pn (Mannose-6 phosphonate)
PBS (Phosphate buffered saline; ThermoFisher)

### FACS Buffer

In 1×PBS
2% FBS (Invitrogen), 2 mM EDTA (Invitrogen), 25 mM HEPES (Invitrogen)
0.2 μM sterile filtered

### Growth Media

Basal Medium: DMEM+L-Glut+Sodium Pyruvate (Invitrogen)
Additives: 10% FBS (Invitrogen), 1x Anti-Anti (Invitrogen)
0.2 μM Sterile Filtered

### Example 126: CI-M6PR CRISPR KO Generation

This example provides the protocol for generation of M6PR knockout (KO) cells. Cells were washed with PBS and detached using TrypLE. Media was added to the flask to deactivate trypsin. Cells were collected and counted. A total of 1×10⁶ cells was then centrifuged at 300×g for 5 minutes. The cell pellet was washed once with PBS and centrifuged at 300×g for 5 minutes. The cell pellet was resuspended in Lonza SE buffer supplemented with supplement 1 and electroporation enhancer (5 M final). CRISPR RNP reaction began by combining equal volumes of 100 M crRNA and tracrRNA in a PCR tube. Using a thermocycler, this mixture was heated to 95° C. for 5 minutes and allowed slowly to cool to room temperature. The annealed sgRNA product was combined with TrueCut Cas9 and allowed to incubate at RT for 15 minutes. Resuspended cells in SE buffer was mixed with the RNP reaction and allowed to incubate for 5 minutes. The entire reaction contents was then placed in a single well of a 16-well electroporation cuvette. Using a Lonza Amaxa cells were pulsed with code CA-163. After pulsing, cells were plated into a 10 cm dish. Six days post-RNP, a portion of cells were collected and lysates were prepared to test for knock-out by western.

### Example 127: Alexa Fluor Labelling

Cetuximab, matuzumab and human IgG isotype antibodies were conjugated to Alexa Fluor 647 using Alexa Fluor™ 647 Protein Labeling Kit (Invitrogen) per the manufacturer's protocol. In brief, antibodies to be labeled were diluted to 2 mg/mL in PBS to a total volume of 500 μL. A 15 DOL (degree of labeling) was used for the conjugation with the fluorophore. Free dye was removed by pre-wetting an Amicon 30 kDa filter with PBS. After incubation, the conjugation reaction was then added to the filter and spun at high speed for 10 minutes. Retained solution was then resuspended in PBS to a final volume of 1 mL and stored at 4° C. indefinitely.

The above procedure can be adapted to fluorescently label a variety of antibodies or target proteins of interest with alternative dyes such as Alexa Fluor™ 488 using e.g., NHS—lysine conjugation chemistry.

### Example 128: Measurement of EGFR/IgG Levels by Surface Staining

This example provides a protocol for the measurement of the time course activity of cetuximab-(Compound A), cetuximab-(Compound 1-7), matuzumab-(Compound A), and matuzumab-(Compound 1-7) conjugates on surface EGFR and IgG levels in Hela parental and M6PR KO cells measured by surface staining.

### Day −1

1e6 Hela parental or M6PR KO cells were plated in 2 mL of media in 6 well plates.

### Day 0

Media was replaced with 1.5 mL of fresh media.

PBS, unconjugated antibodies and m6P conjugated antibodies were added to respective wells at a final concentration of 20 nM.

### Day 1/2/3

Media was aspirated from wells and were washed thrice with PBS. 750 μL of Enzyme-Free Dissociation buffer was added and cells were allowed to detach on ice.

Cell were collected in tubes and spun down at 300×g for 5 mins @4° C.

Cells were resuspended in PBS and volume was split equally into two tubes.

All tubes were spun at 300×g for 5 mins at 4° C. One set, the PBS was aspirated and pellets were frozen at −80° C.

The other set, the PBS was aspirated and washed 2x with cold FACS buffer.

After final wash, pellets were resuspended in 300 μL FACS buffer.

The 300 μL suspension was split into three wells (100 μL each) of a 96 well plate.
Set 1: Ctx::AF647 at 1:100 dilution and incubated on ice in the dark for 1 h.
Set 2: Mtz::AF647 at 1:100 dilution and incubated on ice in the dark for 1 h.
Set 3: Goat anti-human IgG PE at 2 g/mL and incubated on ice in the dark for 1 h.

Cells were spun down at 1000×g at 4° C. for 3 minutes and liquid was decanted. Cell pellets were resuspended in 200 μL of cold FACS buffer. Repeated 3x total.

After final wash and decant, cells were resuspended in 100 μL cold FACS buffer with DAPI (25 ug/mL final).

Stained cells were then analyzed on Biorad ZE5.

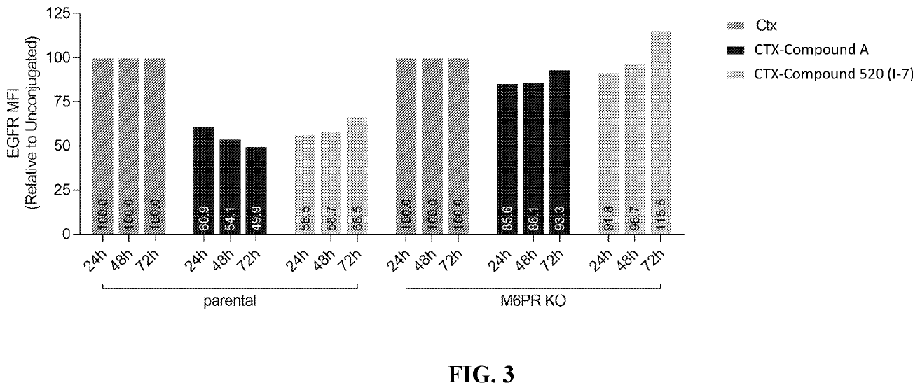

FIG. 3 shows the time course activity of cetuximab-(Compound A) and cetuximab-(Compound 1-7) conjugates on surface EGFR levels in Hela parental and M6PR KO cells measured by surface staining.

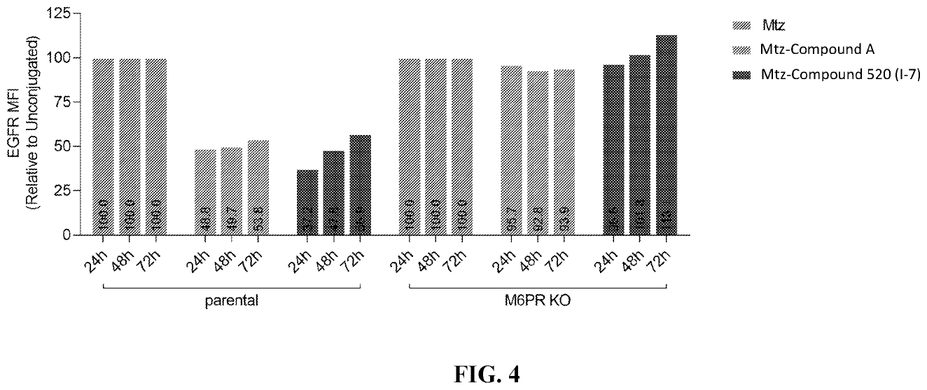

FIG. 4 shows the time course activity of matuzumab-(Compound A) and matuzumab-(Compound 1-7) conjugates on surface EGFR levels in Hela parental and M6PR KO cells measured by surface staining.

These results show that the conjugates described herein can induce reduction in membrane EGFR.

### Example 129: Live-Cell EGFR Surface Staining by Flow Cytometry

This example provides an alternate protocol for the determination of the effect of matuzumab-(Compound A) or matuzumab-(Compound 1-7) conjugates on surface EGFR levels measured by surface staining using flow cytometry.

Hela parental or M6PR (cation-independent mannose 6-phosphate receptor) knockout (M6PR KO) cells were plated in 6 well plates and treated with vehicle (PBS), unconjugated anti-EGFR antibody (matuzumab, Mtz), or matuzumab-(Compound A) or matuzumab-(Compound 1-7) conjugates for the indicated period of time.

After incubation, media was aspirated and cells were washed three times with PBS, lifted using Accutase and pelleted by centrifugation at 300×g for 5 minutes. Cells were resuspended in cold FACS buffer and kept cold for the remainder of the staining procedure. A portion of cells were excluded from staining procedure as an unstained control. Cells were stained with either human IgG Isotype-AF647 or cetuximab-AF647 conjugates for 1 hour at on ice in the dark. Cells were then spun at 300×g for 5 min at 4° C. and washed with cold FACS buffer for a total of three washes. After the final wash, cells were resuspended in 100 μL of FACS buffer with DAPI added at a final concentration of 5 g/mL. Cells were analyzed using a BioRad ZE5 flow cytometer and data was analyzed using FlowJo software. Cells were first gated to remove debris, doublets and dead cells (DAPI negative). EGFR cell surface levels were determined based on AF647 mean fluorescence intensity (MFI).

In parental Hela cells, treatment with the M6Pn-conjugated antibodies (cetuximab-(Compound A), cetuximab-(Compound 1-7), matuzumab-(Compound A), and matuzumab-(Compound 1-7)) resulted in reduced cell surface levels of EGFR compared to cells treated with unconjugated antibodies (Ctx or Mtz). The reduction in cell surface EGFR was dependent on M6PR as they did not occur in M6PR knockout (M6PR KO) cells.

These results show that treatment of cells with the conjugates described herein can induce reduction in targeted cell surface receptors.

### Example 130: Measurement of Total EGFR Levels by Western Blotting

This example provides the protocol for the measurement of the time course activity of cetuximab-(Compound A), cetuximab-(Compound 1-7), matuzumab-(Compound A), and matuzumab-(Compound 1-7) conjugates on total EGFR levels in Hela parental and M6PR KO cells measured by traditional Western blotting.

Once all time-points from Example above were collected, all cell pellets were resuspended in 50 μL of radioimmunoprecipitation assay (RIPA) buffer (+protease/phosphatase inhibitor+nuclease).

Lysates were incubated on ice for 1 h.

Lysates were then spun at high-speed for 10 min at 4° C.

40 μL of cleared lysate was transferred to a 96 well plate.

All lysate concentrations were calculated using BCA assay (1:3 dilution).

All lysates were equalized to 2 mg/mL using RIPA as diluent.

Equal volumes (15 L) of lysate were then mixed with LDS sample buffer (3×LDS+2.5x reducing agent).

Samples were incubated at 98° C. for mins and allowed to cool.

Samples were vortexed and spun down.

15 μL of sample was loaded onto a 26-well bis-tris 4-12% midi-gel.

Gel was allowed to run at 180V for 20 mins.

Gels were transferred to nitrocellulose membrane using iBlot 2 (20V constant, 7 mins).

Membranes were washed 1× in PBS and then placed in Odyssey blocking buffer for 1 h RT with shaking.

Primary antibodies mouse anti-3-actin (SCB) and rabbit anti-EGFR (CST) were diluted 1:1000 in blocking buffer and allowed to incubate overnight at 4° C. with shaking.

Membranes were washed thrice with PBS-T (Tween20 0.1%), at least 5 mins each wash.

Secondary antibodies anti-mouse 680rd and anti-rabbit 800cw were diluted 1:5000 in blocking buffer and allowed to incubate for 1 h at RT with shaking.

Membranes were washed thrice with PBS-T (Tween20 0.1%), at least 5 mins each wash.

Membranes were imaged using licor odyssey scanner.

### Example 131: Measurement of Total EGFR Levels by in-Cell Western Blotting

This example provides a protocol for the measurement of the dose response of cetuximab-(Compound A), cetuximab-(Compound I-7), matuzumab-(Compound A), and matuzumab-(Compound I-7) conjugates on total EGFR levels in Hela parental and M6PR KO cells measured by in-cell Western blotting.

### Day −1

3e4 Hela parental or M6PR KO cells were plated 100 μL per well in a clear bottom black walled 96 well plate (Costar 3603)

### Day 0

Media was decanted and 100 μL of fresh media was added back to wells.

50 μL of a 3× dose response of unconjugated and m6P conjugated antibodies were added to respective wells.

80 nM final starting concentration, 1:2 dilution. EGF was added at in 3 wells at a concentration of 50 ng/mL final.

### Day 2

Media was decanted and wells were washed thrice with PBS.

Wells were fixed with 4% PFA in PBS for 15 minutes at RT.

Wells were washed thrice with PBS.

Cells were permeabilized with 0.2% triton-x100 in PBS for 15 mins. Repeated 3× total.

Cells were blocked in Odyssey blocking buffer with 0.2% triton-x100 for 1 h at RT.

Cells were stained with goat anti-EGFR (AF231, R&D, 1 g/mL final) in block buffer overnight at 4° C.

Cells were washed 3× with PBS-T (Tween20 0.1%).

Cells were stained with donkey anti-goat 800CW secondary (1:200) and CellTag700 (1:500) in blocking buffer for 1 h at RT in dark.

Cells were washed 3× with PBS-T (Tween20 0.1%).

Last wash was decanted and plates were blotted on paper towel to remove residual liquid.

Plates were imaged on Licor scanner (3 mm offset).

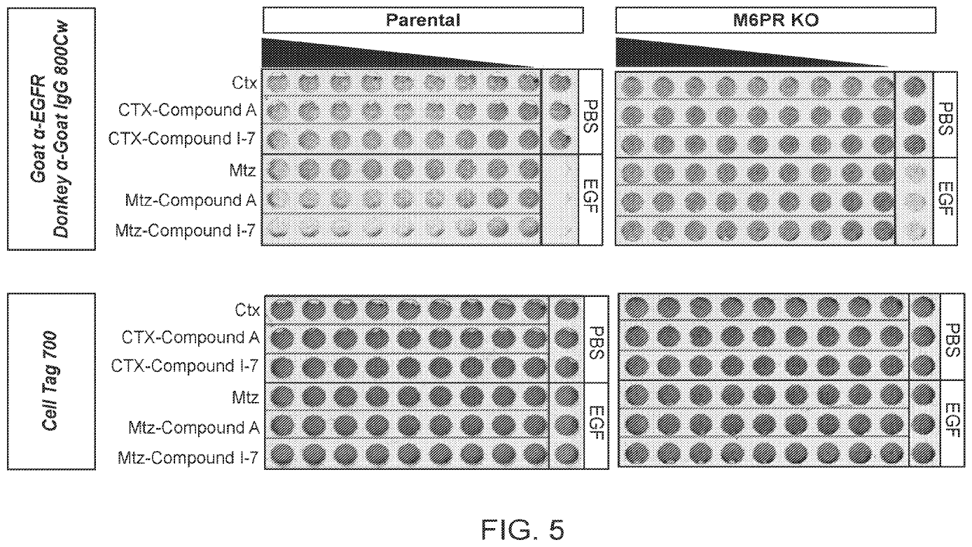

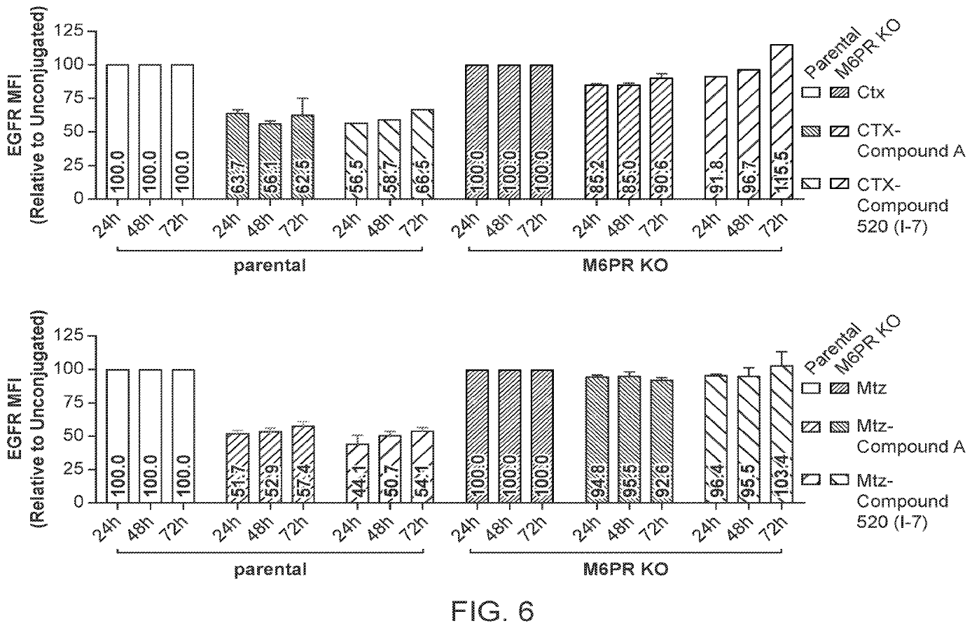

FIG. 5 shows the dose response of cetuximab-(Compound A), cetuximab-(Compound I-7), matuzumab-(Compound A), and matuzumab-(Compound I-7) conjugates on total EGFR levels in Hela parental and M6PR KO cells measured by in-cell Western blotting.

M6Pn-conjugated anti-EGFR antibodies (cetuximab-(Compound A), cetuximab-(Compound I-7), matuzumab-(Compound A), and matuzumab-(Compound I-7)) showed dose-dependent reduction in cellular EGFR compared to unconjugated antibodies alone. The reduction in EGFR was dependent on M6PR as it was observed in parental Hela cells, but not in cells lacking M6PR (M6PR KO).

These results are consistent with those of the Example above, and show that treatment of cells with the conjugates described herein can induce reduction (e.g., degradation) in targeted cell surface receptors.

### Example 132: Measurement of Cellular EGFR Protein Levels Evaluated by Immunocytochemistry

This example provides an alternate protocol for the determination of the effect of M6Pn-conjugated anti-EGFR antibodies (either Mtz or Ctx) on cellular EGFR protein levels evaluated by immunocytochemistry.

HeLa parental or M6PR (cation-independent mannose 6-phosphate receptor) knockout (M6PR KO) cells were plated in 6 well plates and treated with vehicle (PBS), unconjugated anti-EGFR antibody (matuzumab, Mtz), or matuzumab-(Compound A) or matuzumab-(Compound I-7) conjugates at 37° C. for 24 hours. After incubation, media was aspirated and cells were washed thrice with PBS. Cells were fixed with 4% PFA for 10 minutes at room temperature, washed three times with PBS and then blocked with 5% BSA in PBS for 1 hour at RT. Cells were permeablizied with 0.2% Triton-X100 in PBS for 15 minutes. After washing, cells were stained with goat anti-EGFR (AF321; R&D Systems) in blocking buffer overnight at 4 C. After washing, cells were stained with anti-goat 800CW secondary or CellTag700, and imaged on Licor scanner.

M6Pn-conjugated anti-EGFR antibodies (cetuximab-(Compound A), cetuximab-(Compound I-7), matuzumab-(Compound A), and matuzumab-(Compound I-7)) showed dose-dependent reduction in cellular EGFR compared to unconjugated antibodies alone. The reduction in EGFR was dependent on M6PR as it was observed in parental Hela cells, but not in cells lacking M6PR (M6PR KO). These results are consistent with those of Examples above, and show that treatment of cells with the conjugates described herein can induce reduction (i.e., degradation) of targeted cell surface receptors.

### Example 133A: Human CI-M6PR Binding Assay

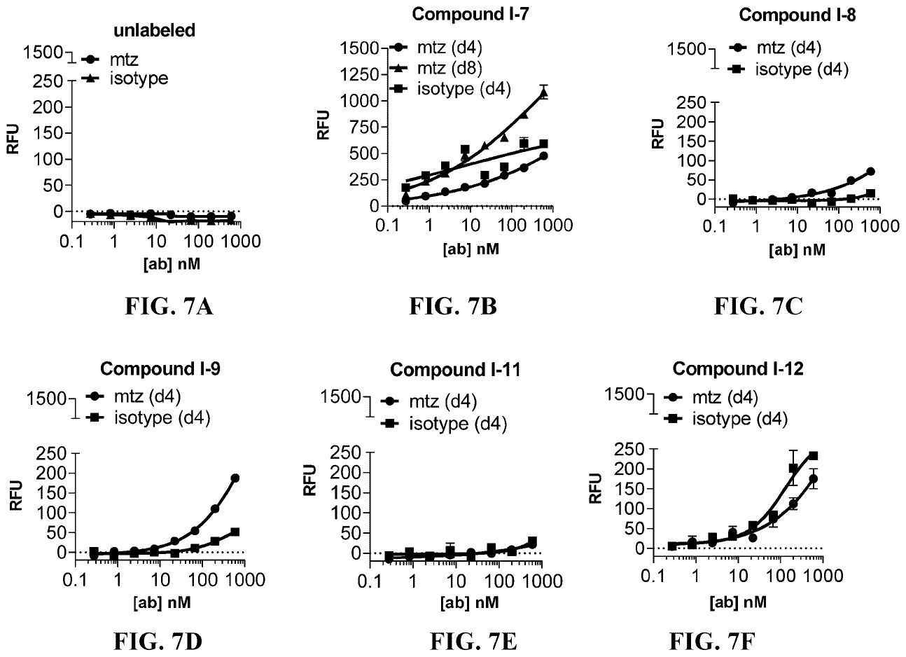

Nunc black solid bottom MaxiSorp plates were allowed to incubate overnight at 4° C. coated with 1 μg/mL of recombinant human CI-M6PR protein (R&D, 6418-GR-050) in 50 μL PBS. The next day, coating was decanted and plates were washed 3× with PBS. Wells were blocked with 350 μL of 3% BSA-PBS for 1 hour at room temperature. Blocking solution was removed and matuzumab conjugates (matuzumab-Compound I-7(d4), matuzumab-Compound I-7(d8), matuzumab-Compound I-8(d4), matuzumab-Compound I-9(d4), matuzumab-Compound I-11(d4) and matuzumab-Compound I-12(d4)) and their respective isotype controls (human IgG (bioxcell, BP0297) conjugated to the ligand-linker compounds being tested) were diluted in 3% BSA-PBS. 50 μL of diluted conjugates were added to the plate and allowed to incubate at room temperature for 2 hours. After incubation, solutions in plate were decanted and washed with 350 μL of 0.05% PBS-Tween20 three times, drying the plate each wash on a clean paper towel. 50 μL of peroxidase AffiniPure Mouse Anti-Human IgG (Jackson Immuno, 209-035-088) diluted in 3% BSA-PBS to 0.2 g/mL was added to the plate and allowed to incubate for 1 hour at room temperature in the dark. After incubation, solutions in plate were decanted and washed with 350 μL of 0.05% PBS-Tween20 3 times, drying the plate each wash on a clean paper towel. QuantaBlu fluorogenic peroxidase substrate (ThermoFisher, 15169) was prepared per manufacturer's suggestions and equilibrated to room temperature. 50 μL of QuantaBlu solution was added to wells and allowed to incubate for 5-10 minutes at room temperature. After incubation, plates were read on a Perkin Elmer EnVision using photometric 340 and Umbelliferone 460 filter sets for excitation and emission, respectively. Data analysis and non-linear curve-fitting was performed using GraphPad Prism. FIGS. 7A-7F show various binding affinities of the conjugates tested, with Compound I-7 (d8) and Compound I-11 (d4) displaying the highest and lowest binding affinity, respectively.

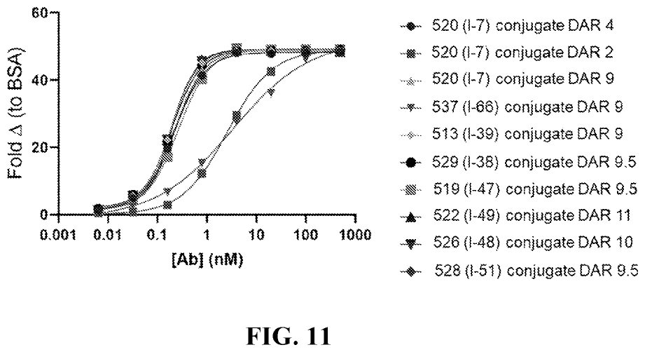

FIG. 11 shows a graph of results of a M6PR binding assay for a variety of antibody conjugates of exemplary compounds with various DAR average loadings. The EC₅₀ values of FIG. 11 are shown in Table 16A. Further results from additional M6PR binding assays are shown in Tables 16B.

TABLE 16A
EC50 values in M6PR binding assay

| Conjugate of compound # | Average Loading DAR | EC50 (nM) |
|---|---|---|
| 520 (1-7) | 2 | 2.60 |
| 520 (1-7) | 4 | 0.22 |
| 520 (1-7) | 9 | 0.22 |
| 537 (1-66) | 9 | 3.36 |
| 513 (1-39) | 9 | 0.19 |
| 529 (1-38) | 9.5 | 0.19 |
| 519 (1-47) | 9.5 | 0.27 |
| 522 (1-49) | 11 | 0.23 |
| 526 (1-48) | 10 | 0.19 |
| 528 (1-51) | 9.5 | 0.20 |

TABLE 16B
EC50 values in M6PR binding assay

| Conjugate of compound # | Average Loading DAR | EC50 (nM) |
|---|---|---|
| 520 (1-7) | 4 | 0.41 |
| 728 (1-52) | 6 | 0.28 |
| 528 (1-51) | 2 | 4.44 |
| 528 (I-51) | 8 | 0.30 |
| 537 (1-66) | 8 | 2.31 |
| 706 (1-41) | (maleimide-Cys conjugation) | 0.27 |

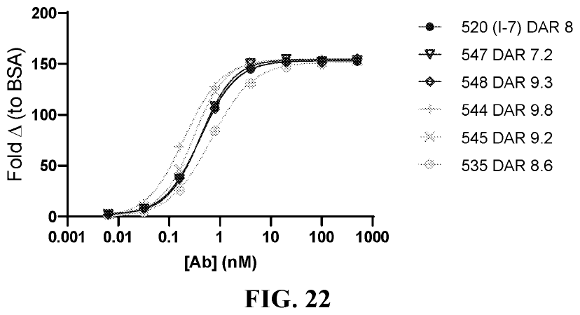

FIG. 22 shows a graph of M6PR binding affinity data for various exemplary cetuximab (anti-EGFR) conjugates. EC50 values calculated based on this data are shown in Table 16C.

TABLE 16C
EC50 values in M6PR binding assay

| Conjugate of compound # | Average Loading DAR | EC50 (nM) |
|---|---|---|
| 520 (1-7) | 8 | 0.41 |
| 547 (1-90) | 7.2 | 0.41 |
| 548 (1-91) | 9.3 | 0.41 |
| 544 (1-86) | 9.8 | 0.20 |
| 545 (1-87) | 9.2 | 0.30 |
| 535 (1-88) | 8.6 | 0.67 |

Table 16D shows M6PR binding affinity data (EC50 nm) comparing various exemplary conjugates having different valencies.

TABLE 16D
EC50 values in M6PR binding assay for conjugates
of compounds of varying multivalency

| Conjugate of | | | m | |
|---|---|---|---|---|
| compound # | X | n | Average Loading DAR | EC50 (nM) |
| 520 (1-7) | X2 | 1 | 2 | 3.71 |
| 520 (1-7) | X2 | 1 | 4 | 0.30 |
| 520 (1-7) | X2 | 1 | 9 | 0.23 |
| 528 (I-51) | X2 | 1 | 2 | 2.23 |
| 528 (1-51) | X2 | 1 | 9.5 | 0.20 |
| 704 (1-40) | X2 | 2 | 2 | 0.66 |
| 704 (1-40) | X2 | 2 | 7 | 0.20 |
| 718 (1-45) | X2 | 3 | 2 | 0.30 |
| 718 (1-45) | X2 | 3 | 7 | 0.22 |
| 763 (1-52) | X2 | 4 | 2 | 0.30 |

TABLE 16E
EC50 values in M6PR binding assay for conjugates
of monovalent compounds of varying linker length and loading

| Conjugate of | | L | m | |
|---|---|---|---|---|
| compound # | X | length | Average DAR | EC50 (nM) |
| 520 (1-7) | X2 | 22 | 2 | 3.71 |
| 520 (1-7) | X2 | 22 | 4 | 0.30 |
| 520 (1-7) | X2 | 22 | 9 | 0.23 |
| 528 (I-51) | X2 | 36 | 2 | 2.23 |
| 528 (I-51) | X2 | 36 | 9.5 | 0.20 |
| 559 | X22 | 22 | 9 | 3.36 |
| 536 (1-60) | X19 | 22 | 9 | 0.19 |
| 529 (1-38) | X3 | 22 | 9.5 | 0.19 |
| 519 (I-47) | X2 | 13 | 9.5 | 0.27 |
| 522 (1-49) | X2 | 19 | 11 | 0.23 |
| 526 (1-48) | X2 | 34 | 10 | 0.19 |

### Example 133B: General M6PR Binding Assay

M6PR binding was measured in black 96-well plates using a fluorescence polarization assay. A fluorescent probe consisting of a reference M6Pn ligand linked to Cy5 was synthesized. Test compounds were resuspended in DMSO and 3-fold serial dilutions were made at 100× final concentrations. Binding reactions were conducted in 100 μl final volume in 20 mM HEPES (pH 7.5) 100 mM NaCl 0.015% o Tween-20 1% DMSO with 100 nM M6PR (Domains 1-9, R&D Systems) and 1 nM probe. Fluorescence polarization was measured using λₑₓ=620 nm, λₑₘ=688 nm on an Envision plate reader (Perkin Elmer) after 2 hr incubation time. Dose responses were conducted in duplicate and normalized to the response with DMSO (high) and 1 μM reference compound (low) on each plate. IC₅₀ values were determined by fitting to 4-parameter curves in GraphPad Prism.

TABLE 16F
EC50 values in M6PR binding assay
IC50(nM) ranges: A ≤ 50 nM; 50 nM < B ≤ 100 nM; 100 nM < C ≤ 500 nM; D ≥ 500 nM

| Compound Structure | IC50 |
|---|---|
| Compound # | (nM) |
| 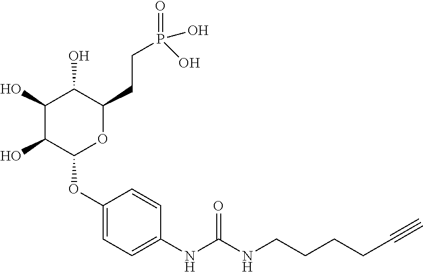 [SMILES: C#CCCCCNC(=O)Nc1ccc(O[C@H]2O[C@H](CCP(=O)(O)O)[C@@H](O)[C@H](O)[C@@H]2O)cc1] | C |
| 38C (X3 monomer) | |
| 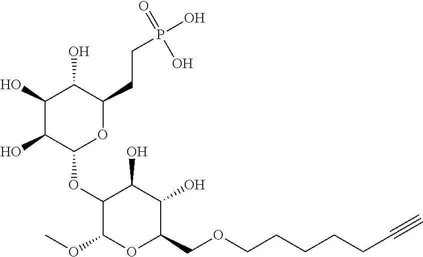 [SMILES: C#CCCCCCOC[C@H]1O[C@H](OC)C(O[C@H]2O[C@H](CCP(=O)(O)O)[C@@H](O)[C@H](O)[C@@H]2O)[C@@H](O)[C@@H]1O] | D |
| I-108 | |
| 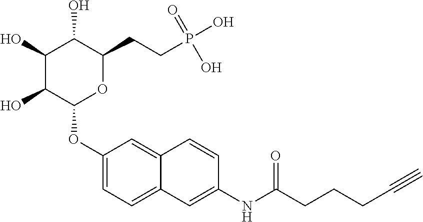 [SMILES: C#CCCCC(=O)Nc1ccc2cc(O[C@H]3O[C@H](CCP(=O)(O)O)[C@@H](O)[C@H](O)[C@@H]3O)ccc2c1] | C |
| I-68 (X9 monomer) | |
| 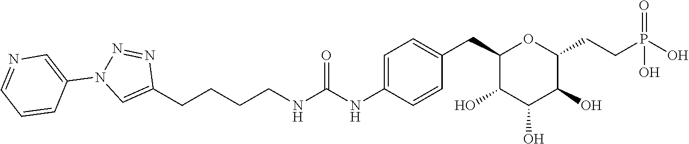 [SMILES: O=C(NCCCCc1cn(-c2cccnc2)nn1)Nc1ccc(C[C@H]2O[C@H](CCP(=O)(O)O)[C@@H](O)[C@H](O)[C@@H]2O)cc1] | C |
| P5 (I-101 + 3-azidopyridine (X27 monomer)) | |
| 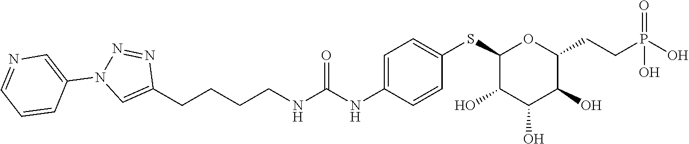 [SMILES: O=C(NCCCCc1cn(-c2cccnc2)nn1)Nc1ccc(S[C@H]2O[C@H](CCP(=O)(O)O)[C@@H](O)[C@H](O)[C@@H]2O)cc1] | C |
| P4 (I-67 + 3-azidopyridine (X11 monomer)) | |
| 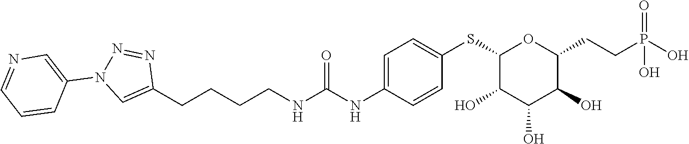 [SMILES: O=C(NCCCCc1cn(-c2cccnc2)nn1)Nc1ccc(S[C@@H]2O[C@H](CCP(=O)(O)O)[C@@H](O)[C@H](O)[C@@H]2O)cc1] | C |
| P3 (I-102 + 3-azidopyridine (X11* monomer)) | |
| 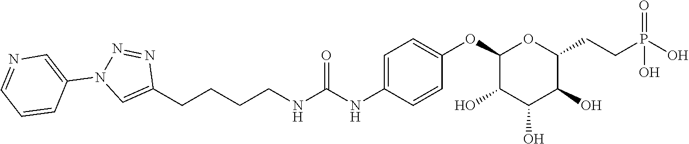 [SMILES: O=C(NCCCCc1cn(-c2cccnc2)nn1)Nc1ccc(O[C@H]2O[C@H](CCP(=O)(O)O)[C@@H](O)[C@H](O)[C@@H]2O)cc1] | C |
| P2 (38C + 3-azidopyridine (X3 monomer)) | |
| 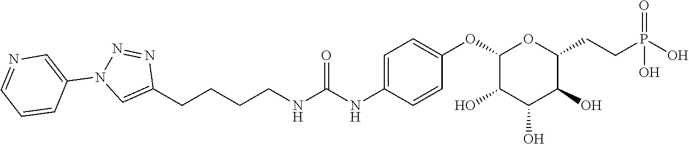 [SMILES: O=C(NCCCCc1cn(-c2cccnc2)nn1)Nc1ccc(O[C@@H]2O[C@H](CCP(=O)(O)O)[C@@H](O)[C@H](O)[C@@H]2O)cc1] | C |
| P1 (566A + 3-azidopyridine (X3* monomer)) | |
| 772 (X3 tetramer, see Table 12B) | B |
| 771 (X3 monomer, see Table 12B) | C |
| 770 (X3 tetramer LC49 + 38C, see Table 12B) | A |
| 769 (X3 dimer. See Table 12B) | C |
| 775 (X3 pentamer, see Table 12B, carboxyl acid not PFP ester) | A |
| 785 (X3 hexamer LC58 + 38C, see Table 12B) | A |
| 781 (X3 dimer LC54 + 38C, see Table 12B) | C |
| 780 (X3 trimer LC51 + 38C, see Table 12B) | A |

These results indicate that binding to M6PR is modulated by ligand structure, linker valency and/or linker geometry.

### Example 134: Serum Pharmacokinetic Analysis for rIgG1 Antibody Conjugates of Varying Binding Affinities

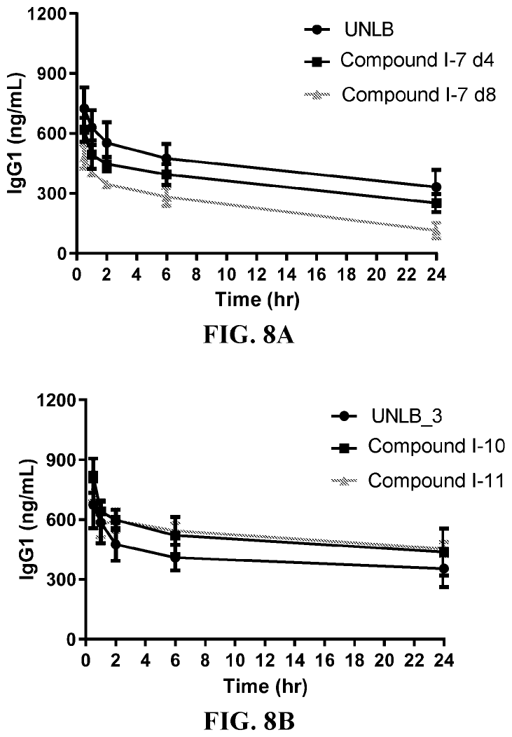

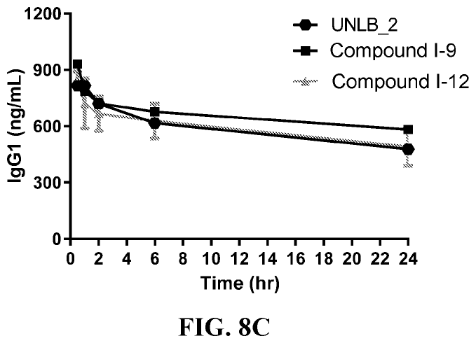

A pharmacokinetic analysis of the rIgG1 (anti-IgG2a) antibody conjugates described in the previous example was performed in mice. In particular, C₅₇B36 mice were intravenously administered each rIgG1 antibody conjugate at 10 μg/mouse (5 mice per group). Blood was collected at 0.5, 1, 2, 6, and 24 hours and serum rIgG1 was analyzed using an ELISA kit (Abcam) according to the manufacturer's instructions. Samples were run across 3 different plates with unconjugated rIgG1 controls (UNLB-anti-IgG2a rIgG1) included on all 3 plates. FIGS. 8A-8C show the serum levels of anti-IgG2a conjugated to Compound 1-7 (dar8) and (dar4) (FIG. 8A), anti-IgG2a conjugated to Compound I-10 and anti-IgG2a conjugated to Compound I-11 (FIG. 8B), and anti-IgG2a conjugated to Compound I-9 and anti-IgG2a conjugated to Compound I-12 (FIG. 8C) over time.

As shown in FIGS. 8A-8C, the PK analysis results demonstrate that conjugates of ligand linkers such as Compounds 1-9, 1-10, I-11, and 1-12 which have weaker binding affinity to M6PR compared to Compound 1-7 exhibit a longer half-life in mice. This result indicates that the ligand may be selected according to a desirable binding affinity useful for tuning the pharmacokinetic properties of the conjugates of this disclosure.

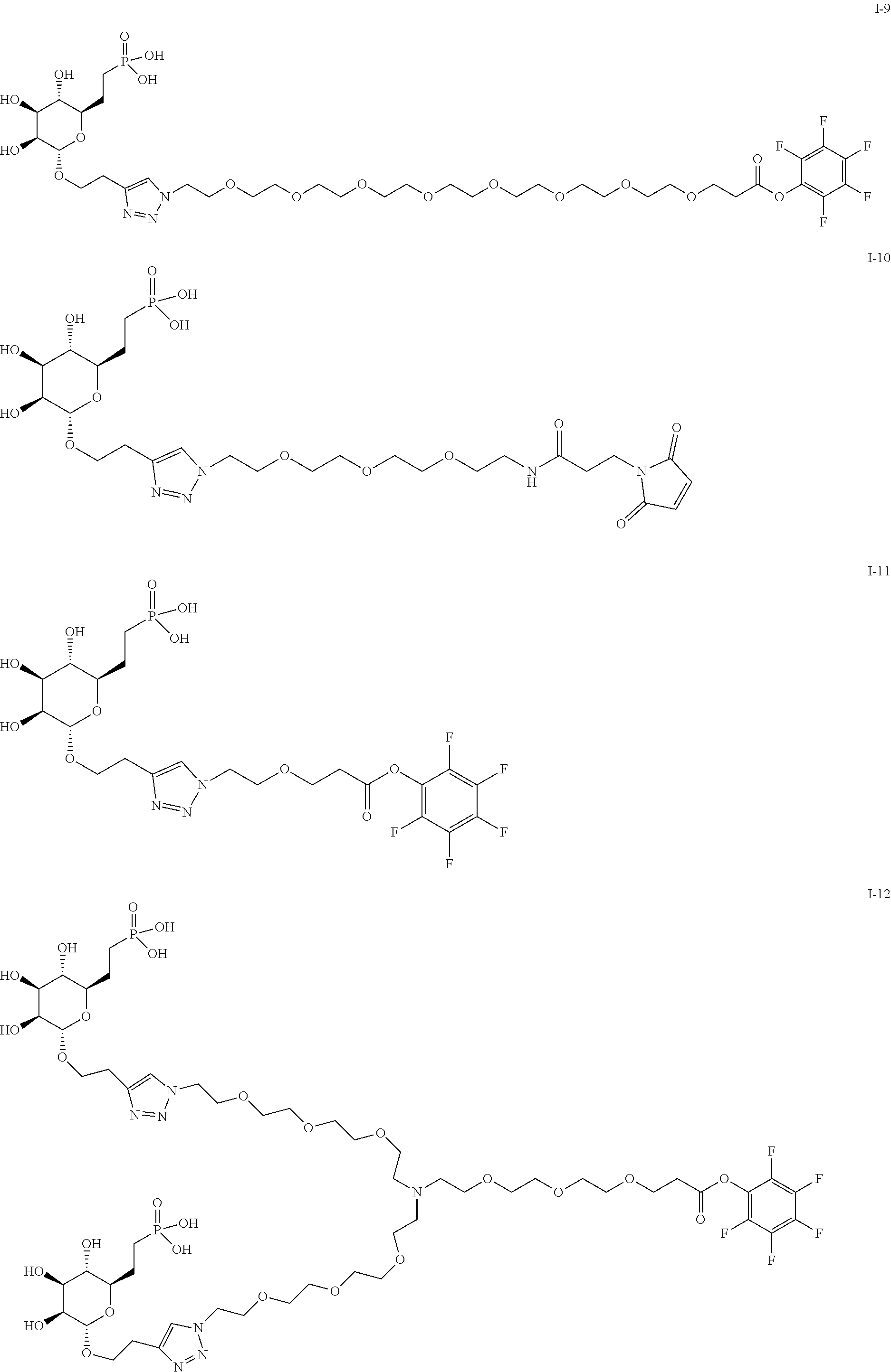 [SMILES: O=C(CCOCCOCCOCCOCCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F ; O=C(CCN1C(=O)C=CC1=O)NCCOCCOCCOCCn1cc(CCO[C@H]2O[C@H](CCP(=O)(O)O)[C@@H](O)[C@H](O)[C@@H]2O)nn1 ; O=C(CCOCCn1cc(CCO[C@H]2O[C@H](CCP(=O)(O)O)[C@@H](O)[C@H](O)[C@@H]2O)nn1)Oc1c(F)c(F)c(F)c(F)c1F ; O=C(CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F]

### Example 135: Conjugates of Varying Binding Affinities Mediate Uptake of IgG2a Into Cells Over Time

The anti-IgG2a conjugates were bound to IgG2a-Alexa488, as follows: equal molar ratios of anti-IgG2a and IgG2a-Alexa488 were added in tissue culture media for 30 minutes at room temperature. The resulting anti-IgG2a:IgG2a antibody-Alexa488 compositions were added to Jurkat cells (100 k cells/50 ul per well, n=2), and Alexa488 fluorescence levels were measured (via Alexa488 measurement) at 1 hour and 24 hours by flow cytometry. As the fluorescently labelled target (IgG2a antibody) accumulates in cells, the fluorescence presents a way to measure total intracellular uptake by cells over time.

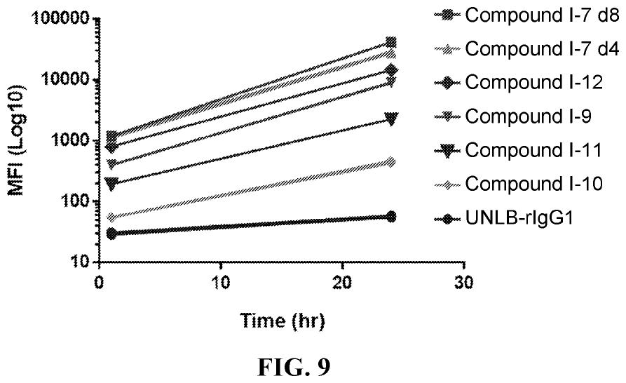

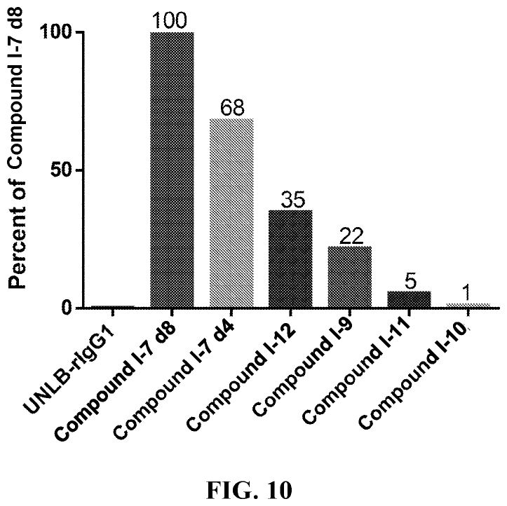

FIG. 9 shows the intracellular levels of aIgG2a conjugates Compound 1-7 (dar8) and (dar4), Compound I-10, Compound I-11, Compound 1-9, and Compound 1-12 at 1 h and 24h. FIG. 10 shows the intracellular uptake of the tested conjugates into Jurkat cells at 10 nM after 24 hours as a percentage of the uptake of aIgG2a conjugate-Compound I-7 (dar8). These data indicate that conjugates of ligand linkers with weaker binding affinity to M6PR than Compound I-7, such as Compounds I-9, I-10, I-11 and I-12, still exhibit sufficiently robust uptake and may therefore be useful for tuning the pharmacokinetic properties of the conjugate, while still capable of mediating uptake.

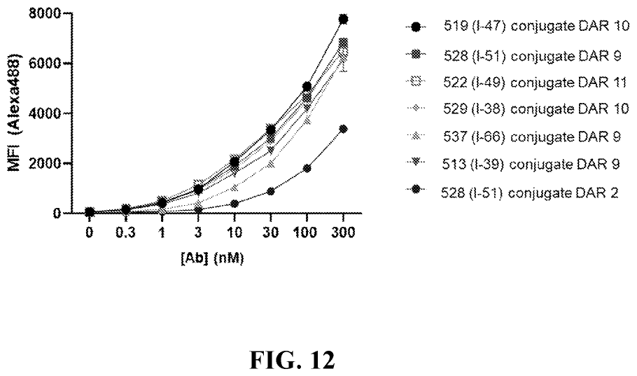

FIG. 12 shows a graph of cell fluorescence versus antibody conjugate concentration indicating that various antibody conjugates of exemplary M6PR binding compounds exhibited robust uptake into Jukat cells after one hour incubation. Conjugates of compounds 519 (1-47) (DAR10), 528 (1-51) (DAR9), 522 (1-49) (DAR11), 529 (1-38) (DAR10), 537 (1-66) (DAR9), and 513 (1-39) (DAR9) all exhibited strong cell uptake. Conjugates of compound 528 (I-51) with average loading DAR9 exhibited greater uptake than conjugate of compound 528 (1-51) with lower average loading DAR2.

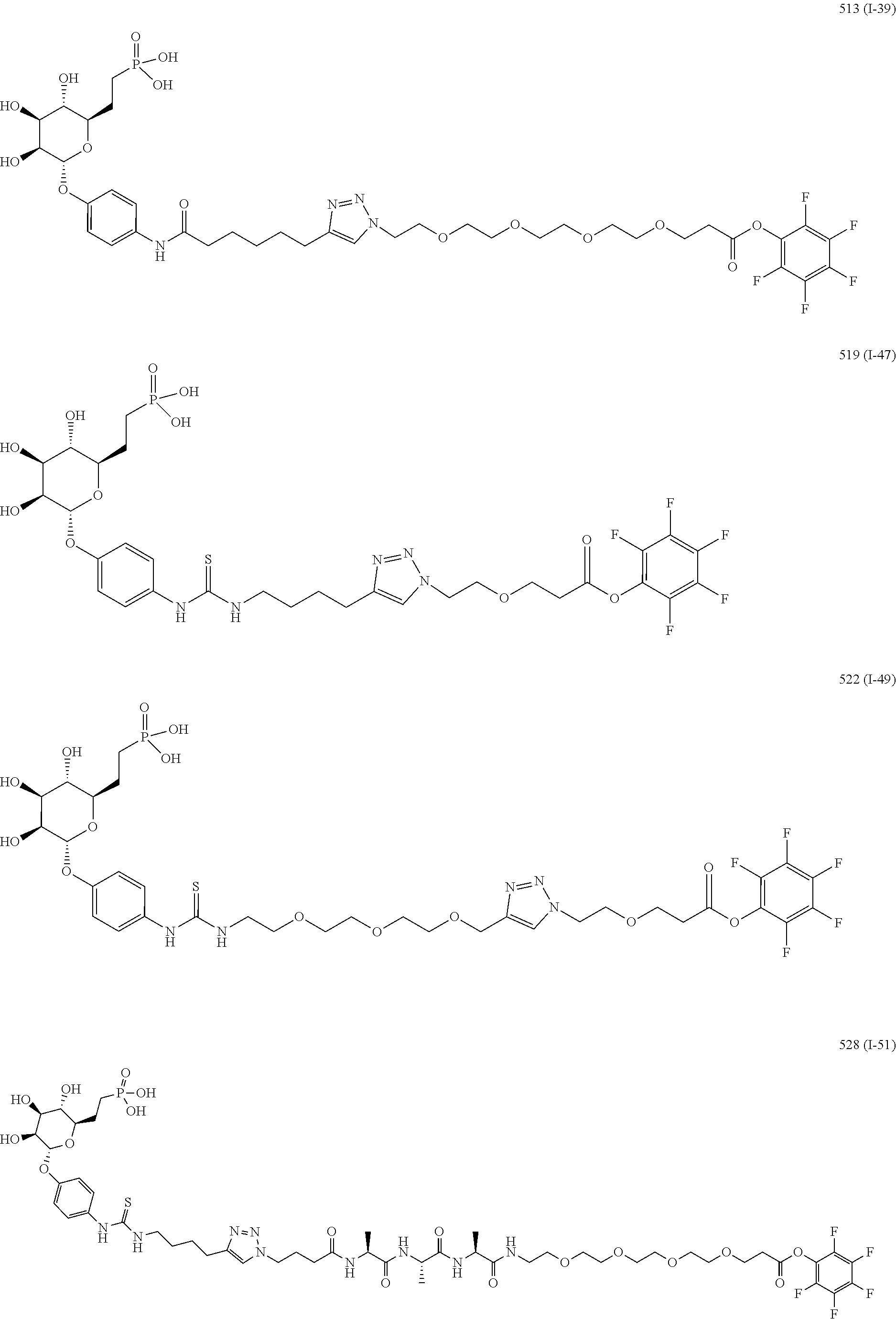

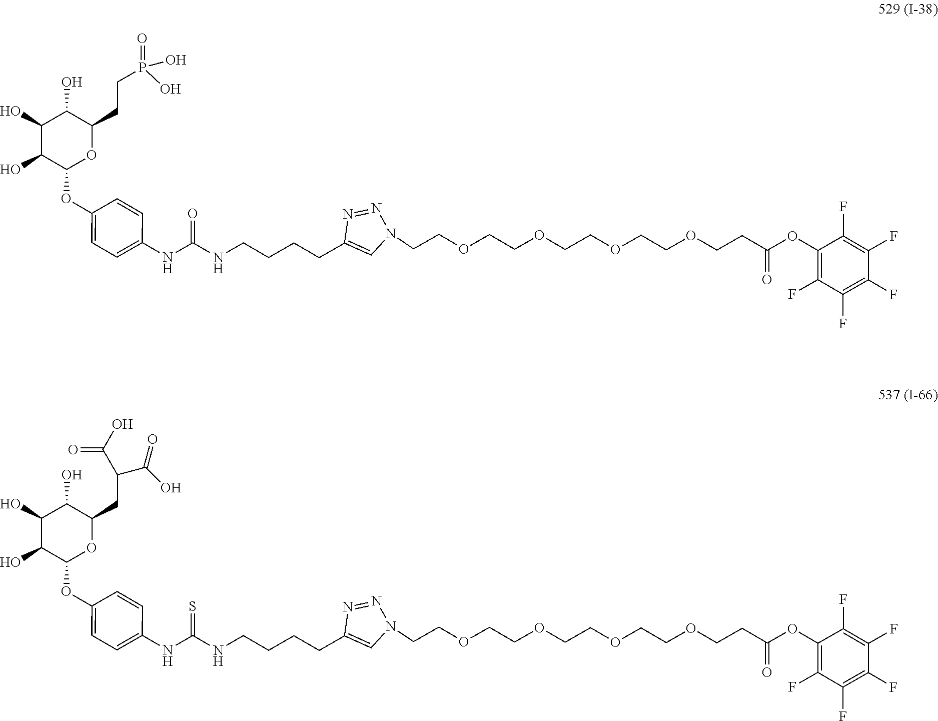

### Example 136: Conjugates of M6PR or ASGPR Binding Compounds Mediate Uptake of IgG2a into Human Liver Cancer Cells

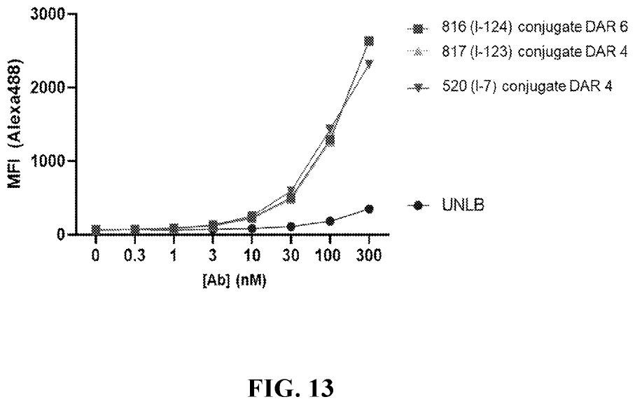

The uptake of antibody conjugates of exemplary M6PR or ASGPR binding compounds was assessed in Hep G2 cells, using a method similar to that described in Example above. FIG. 13 shows a graph of cell fluorescence versus antibody conjugate concentration indicating that various antibody conjugates of exemplary M6PR or ASGPR binding compounds exhibited robust uptake into HepG2 cells after one hour incubation. Conjugates of compound 816 (ASGPR binding compound I-124) (average loading DAR6), compound 817 (ASGPR binding compound 1-123) (average loading DAR4) and M6PR binding compound 520 (1-7) (average loading DAR4) exhibited comparable HepG2 cellular uptake. Compounds 816 and 817 were prepared according to methods described in International Application No. PCT/US2021/012846, filed Jan. 8, 2021.

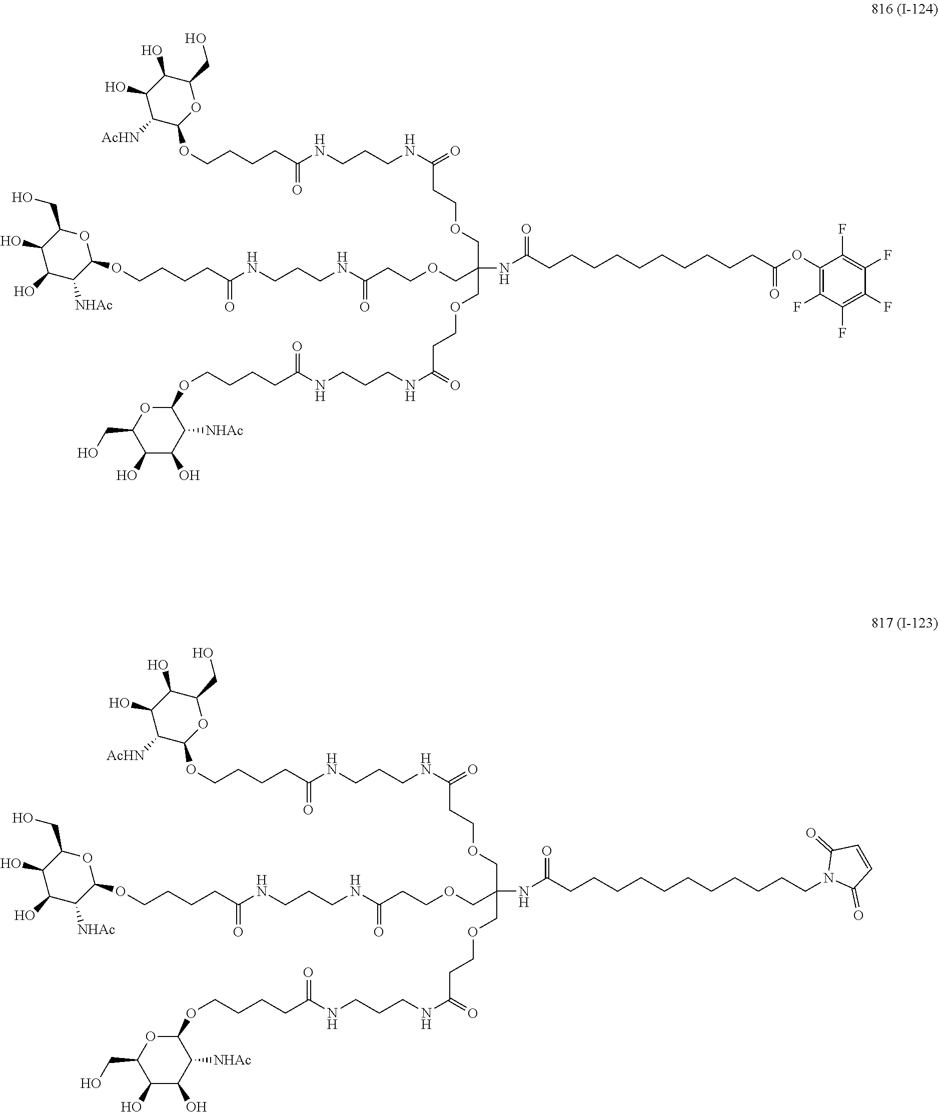

### Example 137. CI-M6PR Mediated Uptake of Target Protein in K562 WT or KO Cells

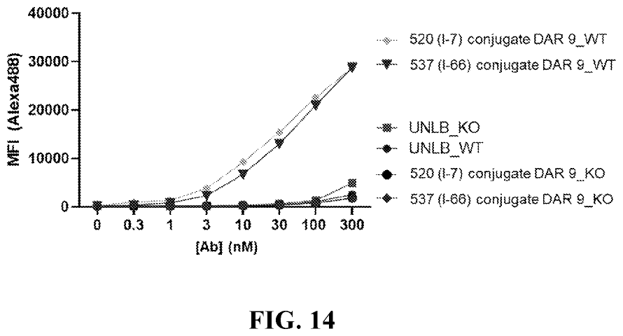

The uptake of exemplary omalizumab (anti-IgE) antibody conjugates of compound 520 (1-7) (average loading DAR9) versus exemplary compound 537 (1-66) (average loading DAR9) was assessed in wild type (WT) K562 cells and CI-M6PR knockout (KO) cells using a similar method of that described above. FIG. 14 shows a graph of the cell uptake of fluorescently labeled Alexa488-IgE target versus an unlabeled control IgE (UNLB) with varying concentrations of conjugate. The cell uptake was shown to be CI-M6PR dependent.

### Example 138. Uptake of Target Protein in Jurkat Cells

The omalizumab conjugates of Example 142 were bound to IgE-Alexa488 (prepared according to Example 144), as follows: equal molar ratios of omalizumab (anti-IgE) conjugate and IgE-Alexa488 were added in tissue culture media for 30 minutes at room temperature. The resulting anti-IgE conjugate:IgE antibody-Alexa488 compositions were added to Jurkat cells (100 k cells/50 ul per well, n=2), and Alexa488 fluorescence levels in the cells were measured at 1 hour by flow cytometry. As the fluorescently labelled target (IgE antibody) accumulates in cells, the fluorescence presents a way to measure total intracellular uptake by cells over time.

FIG. 15 shows a graph of cellular uptake activity of the conjugates of omalizumab (anti-IgE) with exemplary M6PR ligand-linkers of Table 15A, bound to Alexa488 labeled-target IgE in Jurkat cells. Structural details of the conjugates of Table 15A are shown in the following Table 17. The conjugates are ordered in Table 17 according to the relative uptake activity as shown in FIG. 15.

TABLE 17
Omalizumab Conjugates Set 1 (see FIG. 15-17)

| | M6PR ligand | | Length | | |
|---|---|---|---|---|---|
| Ligand-Linker | (see Tables | Linker | (Z3 to Y or | | m |
| (Compd. No.) | 1, 7, 11) | (Table x) | branching atom) | n | (DAR) |
| 520 (1-7) | X2 | 1.5 | 22 | 1 | 4 |
| 520 (1-7) | X2 | 1.5 | 22 | 1 | 8 |
| 529 (1-38) | X3 | 1.5 | 22 | 1 | 4 |
| 558 | X3 | 1.5 | 22 | 1 | 4 |
| 566 | X30 | 1.5 | 22 | 1 | 4 |
| 716 | X17 | See Table 12 | 15 | 3 | 4 |
| 567 | X29 | 7.1 | 15 | 1 | 4 |
| 558 | X22 | 1.5 | 22 | 1 | 4 |
| 556 (I-104) | X27 | 1.5 | 22 | 1 | 4 |
| 665 | see Table 11 | 1.5 | 22 | 1 | 4 |

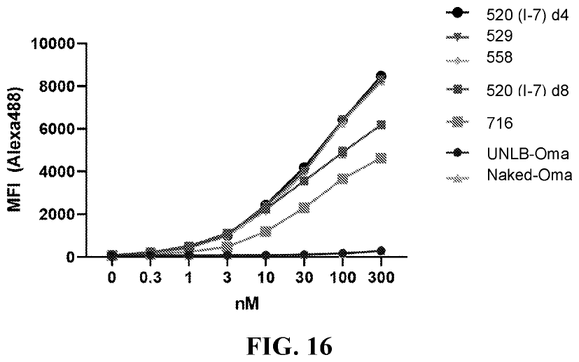

FIG. 16 shows select cellular uptake activity from FIG. 15 illustrating comparisons between selected conjugates. A M6PR ligand containing a urea Z³ group (529 (1-38)) showed comparable activity to a thiourea Z³ analog (520 (1-7) with m=4). A trimeric (n=3, m=4) M6PR ligand (716) conjugate showed inferior uptake to 520 (1-7) with m=8, although the compounds are not directly comparable as they have different M6PR binding moieties.

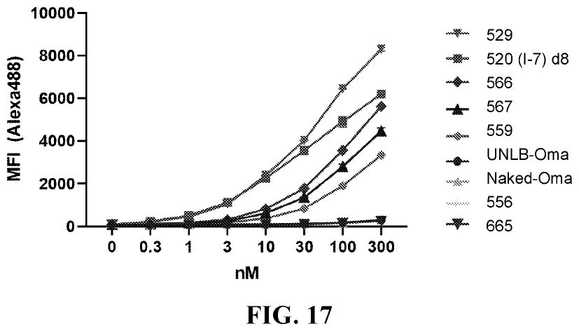

FIG. 17 shows select cellular uptake activity from FIG. 15 illustrating comparisons between selected conjugates. C-glycoside containing M6PR ligand conjugates (compound 566 where Z¹-A is —CH₂-phenyl-versus compound 567 where Z¹-A is —CH₂-triazole-) have similar uptake activity, but are less active than the O-glycoside analog 529 (I-38) (where Z¹-A is —CH₂-phenyl-).

A conjugate containing a M6PR binding moiety having a malonate hydrophilic head group (558) was active in this assay, although less active in comparison to the corresponding analog containing a phosphonate hydrophilic head group (529 (1-38)).

A conjugate containing a M6PR binding moiety containing a cyclohexane ring (665) rather than the pyranose sugar ring of mannose exhibited low activity in this assay.

A conjugate containing a M6PR binding moiety having a sulfonate hydrophilic head group (556 (I-104)) exhibited low activity in this assay.

FIG. 18 shows a graph of cellular uptake of various conjugates of omalizumab (anti-IgE) with exemplary M6PR ligand-linkers of Table 15B, bound to Alexa488 labeled-target IgE in Jurkat cells. The conjugates are ordered in Table 18 according to the relative uptake activity as shown in FIG. 18.

TABLE 18
Omalizumab Conjugate Set 1 (see FIG. 18-21)

| | M6PR ligand | | Length | | |
|---|---|---|---|---|---|
| Ligand-Linker | (see Tables | Linker | (Z3 to Y or | | m |
| (Compd. No.) | 1 and 7) | (Table 4) | branching atom) | n | (DAR) |
| 570 | X11* | L1.5 | 22 | 1 | 4 |
| 713 | X11 | See Table 12 | 17-18 | 2 | 4 |
| 660 | See Table 7 | L1.11 | 21 | 1 | 4 |
| 659 | See Table 7 | L7.2 | 23 | 1 | 4 |
| 552 (1-95) | X11 | L1.5 | 22 | 1 | 6 |
| 520 (1-7) | X2 | L1.5 | 22 | 1 | 8 |
| 552 (1-95) | X11 | L1.5 | 22 | 1 | 4 |
| 716 (1-12) | X17 | See Table 12 | 16 | 2 | 4 |
| 602 (1-8) | X2 | L1.5 | 22 | 1 | 4 |
| 664 | See Table 11 | L7.3 | 26 | 1 | 4 |
| 666 | See Table 11 | See Table 11 | See Table 11 | 1 | 4 |

FIG. 19 shows select cellular uptake activity from FIG. 18 illustrating comparisons between selected conjugates.

The conjugate having an S-glycoside (552, m=6) showed comparable activity to an O-glycoside (520 (I-7) with m=8). The conjugate having an S-glycoside (552) shows more activity when at m=6 loading versus m=4 loading in this assay.

The conjugate having a β—S-glycoside configuration (570) has superior activity to conjugates having a α—S-glycoside configuration (552), and to a conjugate having an α—O-glycoside configuration (520 (I-7), m=8) over the entire dose response range of this assay.

A conjugate having a triazole group connected at the anomeric position (664) showed minimal activity in this assay.

FIG. 20 shows select cellular uptake activity from FIG. 18 illustrating comparisons between selected conjugates. Conjugates of ligands 660 or 659, each having a di-mannose structure with a 2,5-linked pyranose ring connected to the linker, showed potent and comparable activity to conjugate of 520 (I-7).

The conjugate of compound 666 having a linker connected to a malonate head group via the 6-position of the sugar ring showed minimal activity in this assay.

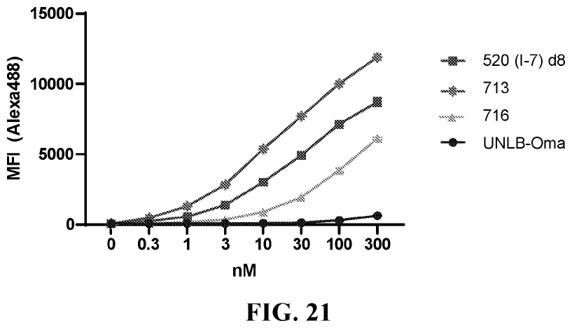

FIG. 21 shows select cellular uptake activity from FIG. 18 illustrating comparisons between selected conjugates. The conjugate of dimeric ligand 701 (I-12) showed activity, but was less active than dimeric ligand conjugate of 713. The conjugate having a dimeric ligand (713) showed superior activity to monomeric ligand (520 (I-7) with m=8) over the entire dose response range.

### Example 139. Assessment of Activity of Target Binding Conjugates of Exemplary β-Glycoside Versus α-Glycoside M6PR Binding Moieties

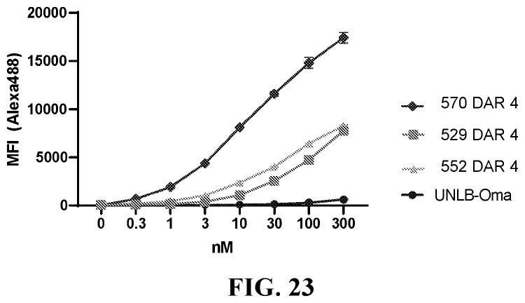

FIG. 23 shows a graph of cellular uptake activity of conjugates of omalizumab (anti-IgE) with exemplary M6PR ligand-linkers of Table 19, versus unlabeled omalizumab (UNLB-Oma) bound to Alexa488 labeled-target IgE in Jurkat cells. Structural details of the conjugates of are shown in the following Table 19. The conjugates are ordered in Table 19 according to the relative uptake activity as shown in FIG. 23. The conjugate of 570 having a β—S-glycoside M6PR binding moiety has superior activity to the corresponding conjugates of α—O-glycoside (529) and α—S-glycoside (552) containing M6PR binding moieties over the entire dose response range. The α—S-glycoside (552) conjugate showed comparable by less activity that the conjugate of α—O-glycoside (529).

TABLE 19
Omalizumab Conjugates Set 1 (see FIG. 23)

| | M6PR ligand | | Length | | |
|---|---|---|---|---|---|
| Ligand-Linker | (see Tables | Linker | (Z3 to Y or | | m |
| (Compd. No.) | 1, 7, 11) | (Table 4) | branching atom) | n | (DAR) |
| 570 | X11* | L1.5 | 22 | 1 | 4 |
| 529 | X3 | L1.5 | 22 | 1 | 4 |
| 552 | X11 | L1.5 | 22 | 1 | 4 |

The CI-M6PR binding affinities of omalizumab (anti-IgE) conjugates of Table 19 were also assessed via SPR binding assay. A conjugate of the β—S-glycoside (570) has superior Kd relative to corresponding α-O (529) and α—S-glycosides (552) at DAR of about 4. At equivalent DAR (m) values, the k_{off} rate of the omalizumab (anti-IgE) conjugates was 552>529>570.

Modelling studies suggest that α—S-glycoside and α—O-glycoside containing M6PR binding moieties adopt a similar axial conformer orientation at the 1-position, while the β—S-glycoside containing M6PR binding moiety can access two low energy equatorial conformers where the 2,3,4-hydroxyl groups can retain a similar receptor binding configuration.

### Example 140. Further Assessment of Degradation Activity of Exemplary Target Binding Conjugates

The target degradation activity of exemplary target binding conjugates is assessed. Applicants have previously demonstrated that bifunctional compounds which demonstrate M6PR mediated cell update of target proteins can also provide for degradation of the target in the lysosome.

The degradation activity can be assessed using a variety of assays related to the target, including for example, a cellular uptake assay, a target protein quantitation assay, or a target activity assay.

The degradation activity of conjugates of matuzimab (anti-EGFR) with exemplary M6PR ligand-linkers (e.g., β—S-glycoside (570) versus α-O (529) and α—S-glycosides (552)) is assessed using an EGFR HiBiT assay.

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A cell surface M6PR binding compound of formula (XIIa): 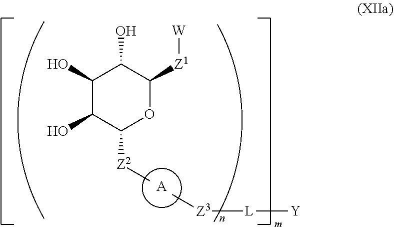
or a prodrug thereof, or a salt thereof,
wherein:
W is a non-hydrolyzable hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
each A is independently an optionally substituted aryl or heteroaryl linking moiety (e.g., monocyclic or bicyclic aryl or heteroaryl, optionally substituted);
each Z³ is independently a linking moiety;
n is 1 to 500;
m is 1 to 100;
L is a linker; and
Y is a moiety of interest;
wherein when A is phenyl and Z² is O, then:
(i) W is —P(O)(OH)₂; or
(ii) the linker L comprises a backbone of at least 16 consecutive atoms and Y is a target binding moiety.

2. A cell surface mannose-6-phosphate receptor (M6PR) binding compound of formula (XIa): 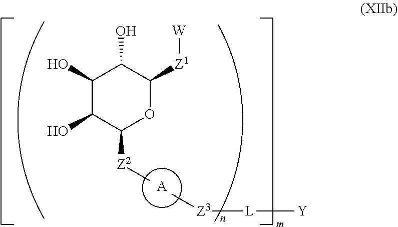
or a prodrug thereof, or a salt thereof,
wherein:
W is a non-hydrolyzable hydrophilic head group;
Z¹ is selected from optionally substituted (C₁-C₃)alkylene and optionally substituted ethenylene;
Z² is selected from O, S, NR²¹ and C(R²²)₂, wherein R²¹ is independently selected from H, and optionally substituted (C₁-C₆)alkyl, and each R²² is independently selected from H, halogen (e.g., F) and optionally substituted (C₁-C₆)alkyl;
A is an optionally substituted cyclic group (e.g., optionally substituted aryl, optionally substituted heteroaryl, optionally substituted heterocycle, or optionally substituted cycloalkyl);
each Z³ is independently a linking moiety;
n is 1 to 500;
m is 1 to 100;
L is a linker; and
Y is a moiety of interest.

3. The compound of claim 2, wherein Z² is S.

4. The compound of claim 2 or 3, wherein W is phosphonate, thiophosphonate, carboxylic or malonic acid, or a salt thereof.

5. The compound of any one of claims 2 to 4, wherein the compound comprises a M6PR binding moiety (X) of one of formula: 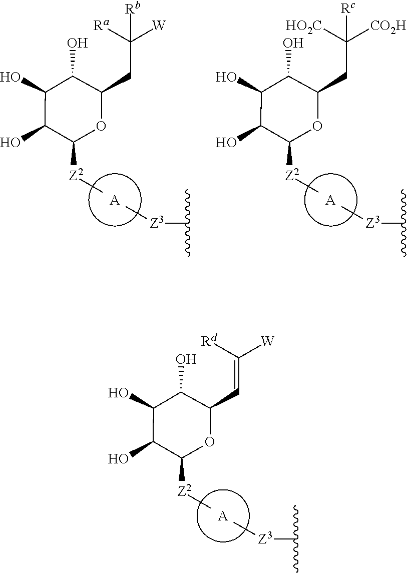
wherein R^{a}, R^{b}, R^{c} and R^{d} are independently H or F.

6. The compound of claim 1, wherein the compound comprises a M6PR binding moiety (X) of one of formula: 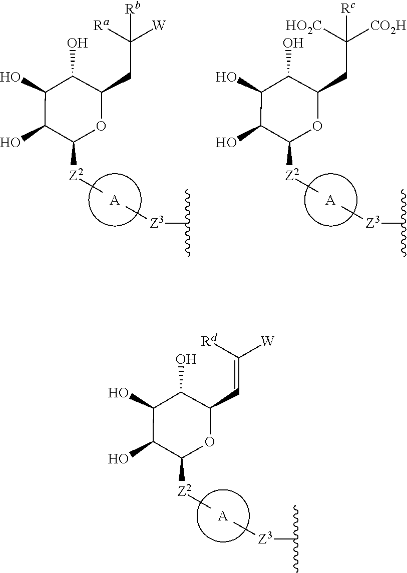
wherein R^{a}, R^{b}, R^{c} and R^{d} are independently H or F.

7. The compound of any one of claims 1-6, wherein A is optionally substituted aryl or optionally substituted heteroaryl, preferably A is independently selected from optionally substituted phenyl, optionally substituted pyridyl, optionally substituted biphenyl, optionally substituted naphthalene, optionally substituted triazole and optionally substituted phenylene-triazole.

8. The compound of claim 7, wherein A is selected from optionally substituted 1,4-phenylene, optionally substituted 1,3-phenylene, optionally substituted 2,5-pyridylene and triazole.

9. The compound of claim 8, wherein A is selected from: 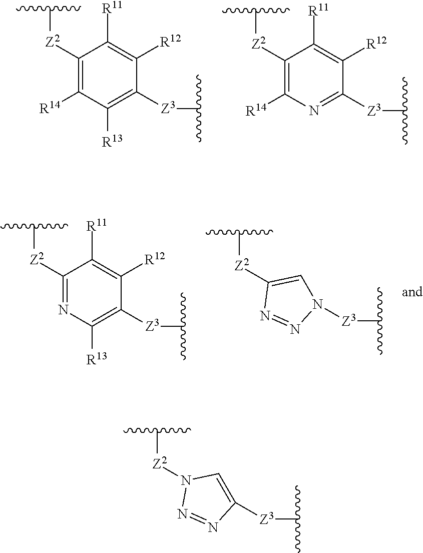
wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
R²⁵ is independently selected from H, and optionally substituted (C₁-C₆)alkyl.

10. The compound of any one of claims 1-6, wherein A is optionally substituted fused bicyclic aryl or optionally substituted fused bicyclic heteroaryl.

11. The compound of claim 10, wherein A is optionally substituted naphthalene or optionally substituted quinoline.

12. The compound of claim 11, wherein A is selected from: 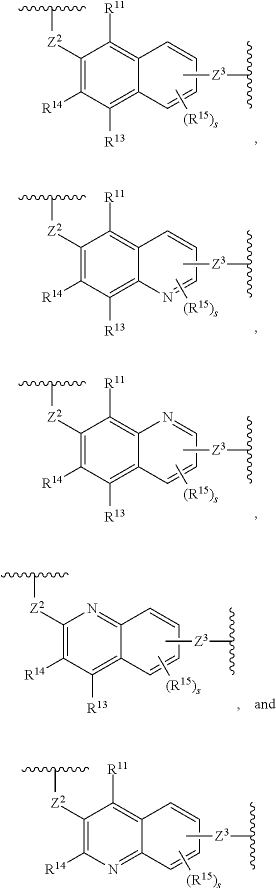
wherein:
R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

13. The compound of claim 12, wherein A is selected from:

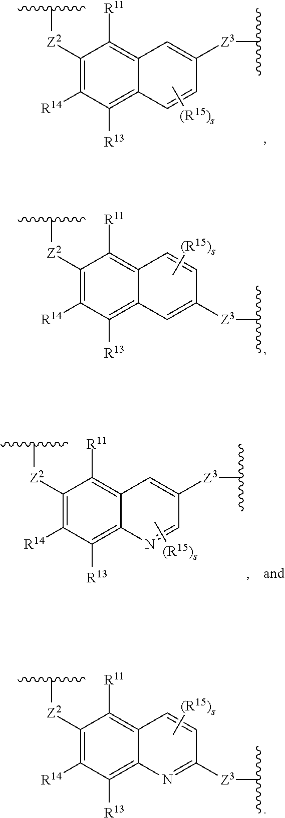

14. The compound of any one of claims 1-6, wherein A is optionally substituted bicyclic aryl or optionally substituted bicyclic heteroaryl of following formula: 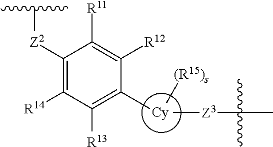
or a salt thereof,
wherein:
Cy is independently monocyclic aryl or monocyclic heteroaryl;
R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²⁵ is independently selected from H, and optionally substituted (C₁-C₆)alkyl.

15. The compound of claim 14 wherein Cy is optionally substituted phenyl, and A is optionally substituted biphenyl of the formula:

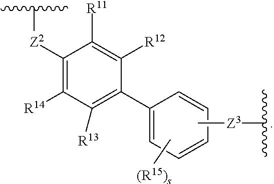

16. The compound of claim 15, wherein A is selected from:

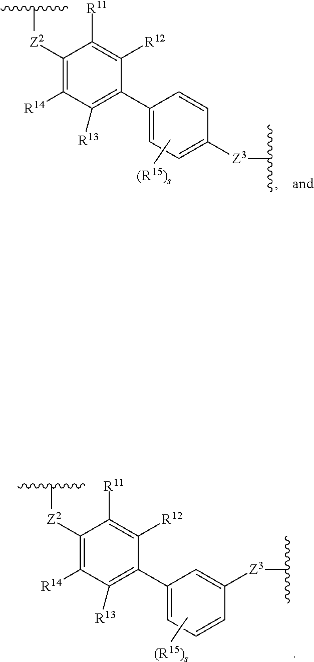

17. The compound of claim 14, wherein Cy is triazole, and A is selected from:

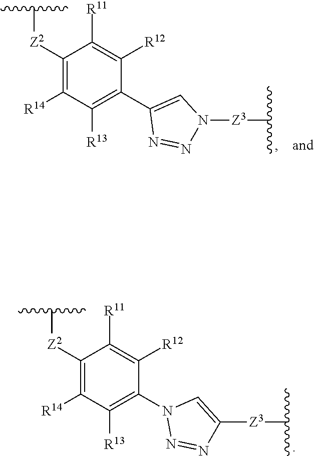

18. The compound of any one of claims 6 to 17, wherein A is substituted with at least one OH substituent.

19. The compound of any one of claims 9, and 12-17, wherein at least one of R¹¹ to R¹⁵ is OH (e.g., at least two are OH).

20. The compound of any one of claims 9, and 12-17, wherein R¹¹ to R¹⁵ are each H.

21. The compound of any one of claims 1 to 20, wherein:
Z³ is selected from a covalent bond, —O—, —NR²³—, —NR²³CO—, —CONR²³—, —NR²³CO₂—OCONR²³, —NR²³C(═X¹)NR²³—, —CR²⁴═N—, —CR²⁴═N—X², —N(R²³)SO₂— and —SO₂N(R²³)—, wherein:
X¹ and X² are selected from O, S and NR²³; and
R²³ and R²⁴ are independently selected from H, C₍₁₋₃₎-alkyl (e.g., methyl) and substituted C₍₁₋₃₎-alkyl.

22. The compound of any one of claims 1 to 21, wherein Z³ is 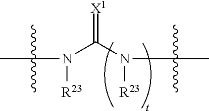
wherein:
X¹ is O or S;
t is 0 or 1; and
each R²³ is independently selected from H, C₍₁₋₃₎-alkyl (e.g., methyl) and substituted C₍₁₋₃₎-alkyl.

23. The compound of claim 22, wherein Z³ is —NHC(═O)NH—.

24. The compound of any one of claims 1 to 23, wherein -A-Z³— is selected from:

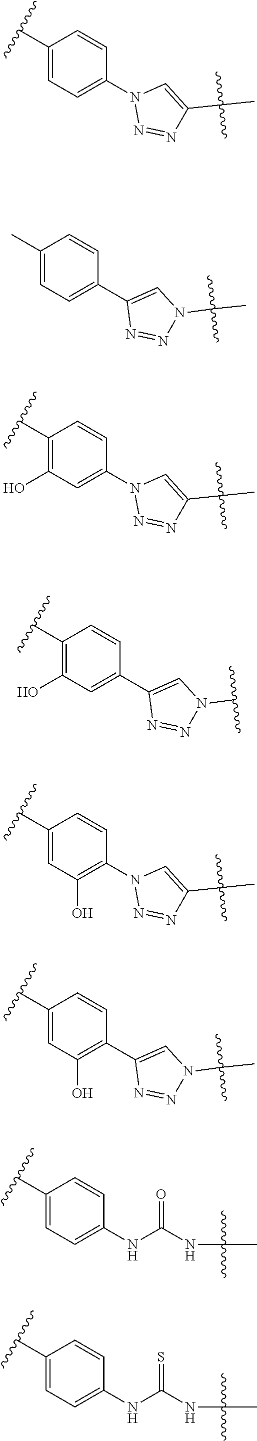

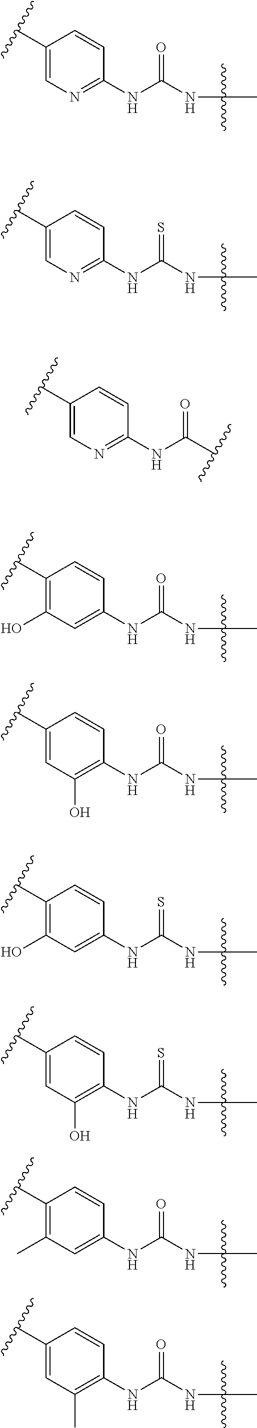

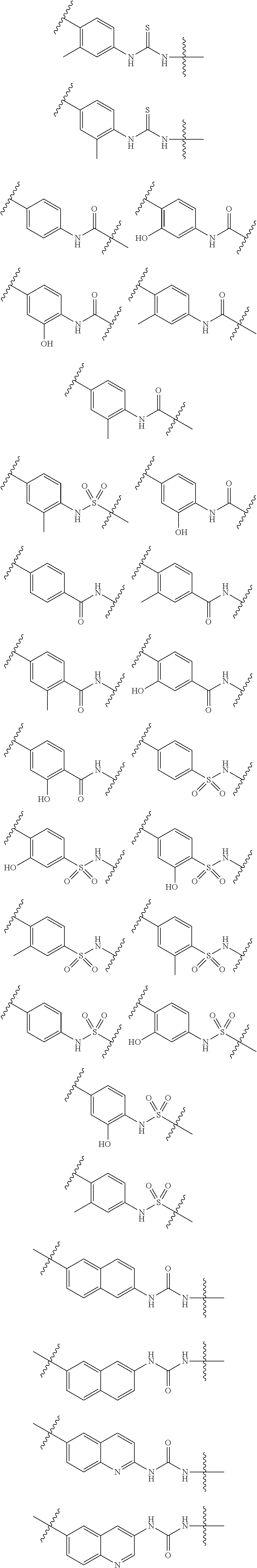

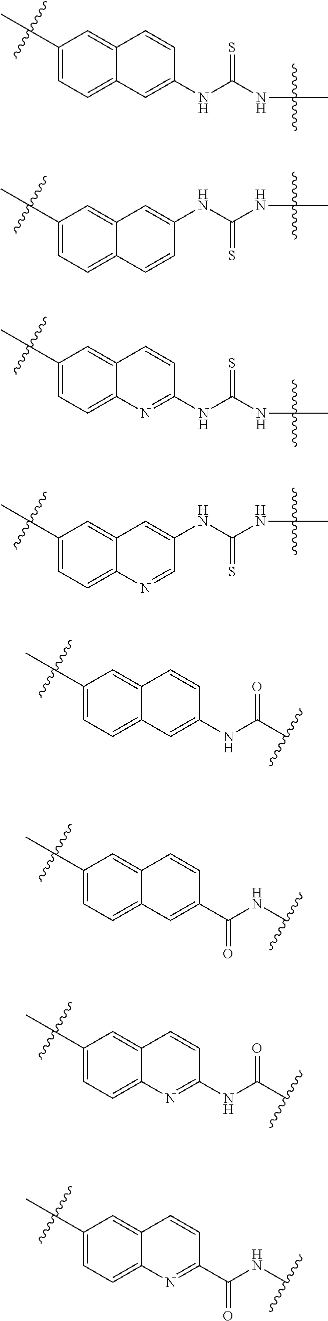

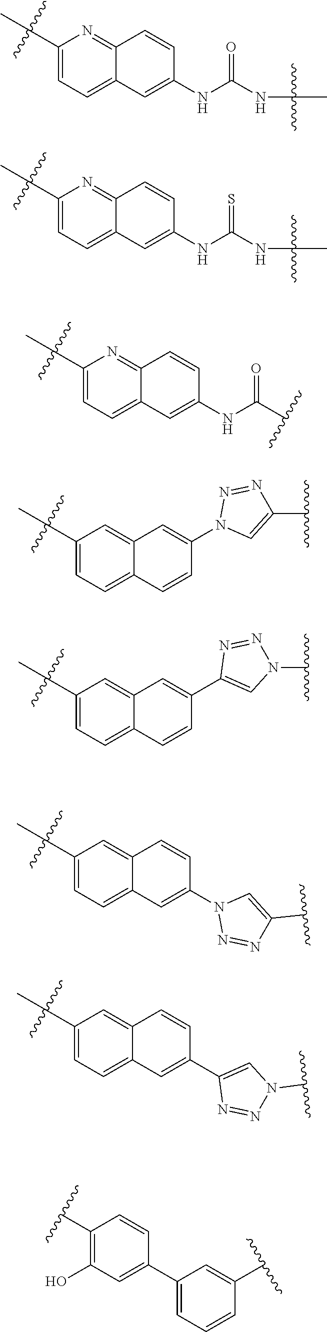

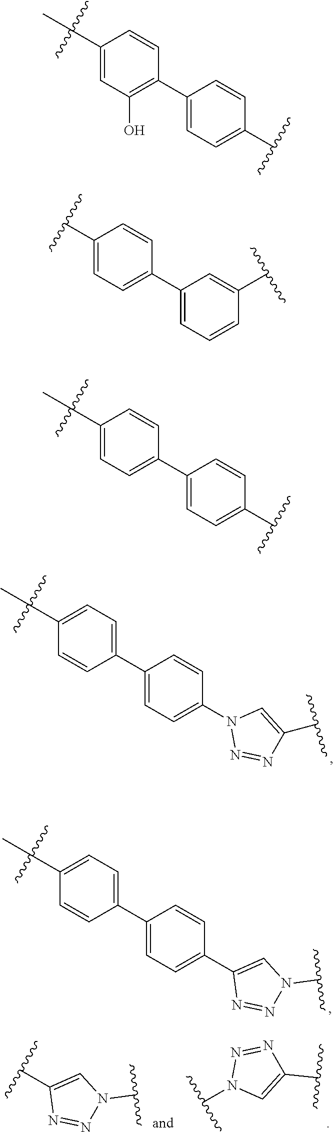

25. The compound of any one of claims 1 and 4-24, wherein Z² is O.

26. The compound of any one of claims 1-24, wherein Z² is S.

27. The compound of any one of claims 1 and 4-24, wherein Z² is —NR²—.

28. The compound of any one of claims 1 and 4-24, wherein Z² is —C(R²²)₂—, wherein each R²² is independently selected from H, halogen (e.g., F) and optionally substituted (C₁-C₆)alkyl.

29. The compound of claim 28, wherein Z² is —CH₂— or —CF₂-.

30. The compound of any one of claims 1 and 5-24, wherein —Z²—Ar—Z³— is 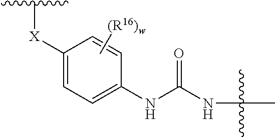
wherein:
X is O, S, —CH₂— or —CF₂;
R¹⁶ is OH; and
w is 0 to 4 (e.g., w is 0, 1, or 2).

31. The compound of claim 30, wherein —Z²—Ar—Z³— is

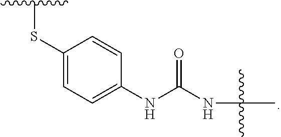 [SMILES: *NC(=O)Nc1ccc(S*)cc1]

32. A cell surface M6PR binding compound of formula (XV): 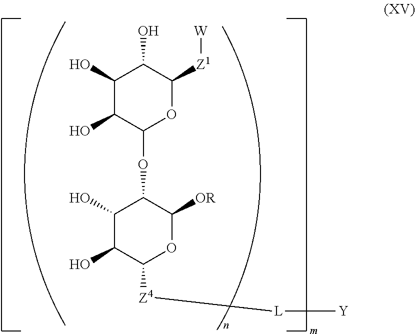
or a prodrug thereof, or a salt thereof,
wherein:
W is a non-hydrolyzable hydrophilic head group;
Z¹ is selected from optionally substituted (C₁-C₃)alkylene and optionally substituted ethenylene;
Z⁴ is selected from —Z¹⁴—, —Z¹⁴-A-, -A-, and —CH₂—Z¹⁴—,
Z¹⁴ is selected from O, S, NR²¹, and C(R²²)₂, wherein R²¹ is independently selected from H, and optionally substituted (C₁-C₆)alkyl, and each R²² is independently selected from H, halogen (e.g., F) and optionally substituted (C₁-C₆)alkyl;
A is an optionally substituted cyclic group (e.g., optionally substituted aryl, optionally substituted heteroaryl, optionally substituted heterocycle, optionally substituted cycloalkyl);
n is 1 to 500;
m is 1 to 100;
L is a linker; and
Y is a moiety of interest.

33. The compound of claim 32, wherein Z⁴ is —CH₂—Z¹⁴—, wherein Z¹⁴ is selected from O, S, NR²¹, and C(R²²)₂.

34. The compound of claim 32, wherein Z⁴ is —CH₂-A-.

35. The compound of claim 32, wherein Z⁴ is -A-.

36. The compound of claim 34 or 35, wherein A is optionally substituted aryl, or optionally substituted heteroaryl.

37. The compound of claim 36, wherein A is triazole.

38. The compound of claim 35, wherein Z⁴ is 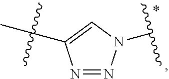 [SMILES: *c1cn(*)nn1]
wherein “*” denotes a connection to the linker L.

39. The compound of any one of claims 1 to 38, wherein the non-hydrolyzable hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —CR¹R²—P═O(OH)₂, —SO₂OH (i.e., —SO₃H), —S(O)OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H, 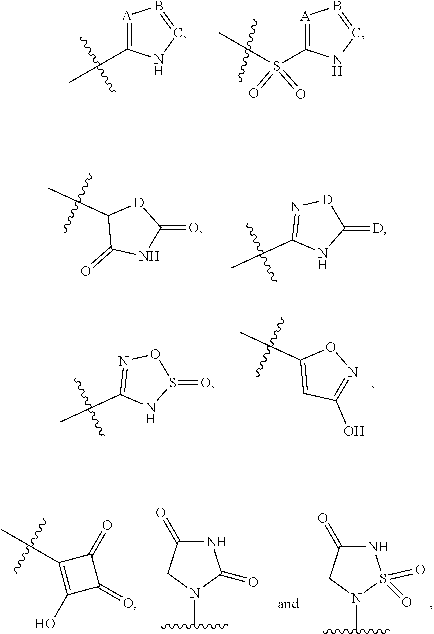
or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

40. The compound of claim 39, wherein W is selected from —P═O(OH)₂, —P═S(OH)₂, —P═O(SH)(OH), —P═S(SH)(OH), —COOH and —CH(COOH)₂, or a salt thereof.

41. The compound of any one of claims 1 to 40, wherein Z¹ is —(C(R²²)₂)ⱼ—, wherein each R²² is independently selected from H, halogen (e.g., F) and optionally substituted (C₁-C₆)alkyl, and j is 1 to 3.

42. The compound of claim 41, wherein Z¹ is —(CH₂)₂—, —CH₂—CF₂—, or —CH₂—CHF—.

43. The compound of claim 41, wherein Z¹ is —CH₂— or —CF₂-.

44. The compound of any one of claims 1 to 40, wherein Z¹ is —CH═CH—.

45. The compound of claim 41, wherein:
Z¹ is —(CH₂)₂—, —CH₂—CF₂— or —CH₂—CHF—; and
W is selected from —P═O(OH)₂, —P═S(OH)₂, —P═O(SH)(OH), —P═S(SH)(OH), and —COOH, or a salt thereof.

46. The compound of claim 60, wherein:
Z¹ is —CH═CH—; and
W is selected from —P═O(OH)₂, —P═S(OH)₂, —P═O(SH)(OH), —P═S(SH)(OH), and —COOH, or a salt thereof.

47. The compound of claim 41, wherein:
Z¹ is —CH₂—, or —CF₂—; and
W is —CH(COOH)₂, or a salt thereof.

48. The compound of any one of claims 1 to 47, wherein n is 1 to 20 (e.g., 1 to 10, 1 to 6, or 1 to 3).

49. The compound of claim 48, wherein n is 1.

50. The compound of claim 49, wherein L comprises a linear linker having a backbone of 16 or more consecutive atoms covalently linking Z³ to Y (e.g., a backbone of 16-100, or 20-100 consecutive atoms).

51. The compound of claim 48, wherein n is 2.

52. The compound of claim 48, wherein n is 3.

53. The compound of any one of claims 1 to 52, wherein L is of formula (II): 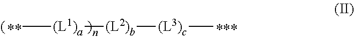
wherein
L¹ and L³ are independently a linker, and L² is a branched linking moiety, wherein L¹ to L³ together provide a linear or branched linker between X and Y;
a, b and c are independently 0 or 1;
** represents the point of attachment to L¹ of X via Z¹; and
*** represents the point of attachment to Y;
wherein:
when n is 1, a is 1, and b is 0;
when n is >1, a is 1, and b is 1.

54. The compound of claim 53, wherein L¹ to L³ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, 1,2,3-triazole, —NH—, and —NMe-, wherein each p is independently 1 to 50.

55. The compound of claim 53 or 54, wherein L comprises repeating ethylene glycol moieties (e.g., —CH₂CH₂O- or —OCH₂CH₂-).

56. The compound of claim 55, wherein L comprises 1 to 25 ethylene glycol moieties (e.g., 3, 7 or 24 ethylene glycol moieties).

57. The compound of any one of claims 53 to 56, wherein L comprises one or more 1,2,3-triazole linking moieties.

58. The compound of claim 57, wherein L comprises one or more linking moieties selected from the following structures: 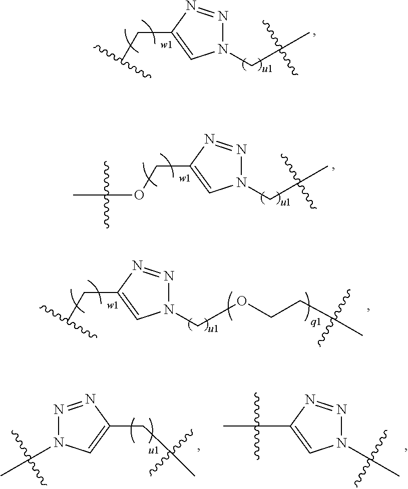
wherein w1, u1 and q1 are independently 1 to 25 (e.g., 1 to 12, such as 1 to 6).

59. The compound of any one of claims 53 to 58, wherein n is 1.

60. The compound of any one of claims 53 to 58, wherein n is 2 or more.

61. The compound of claim 60, wherein L² is selected from: 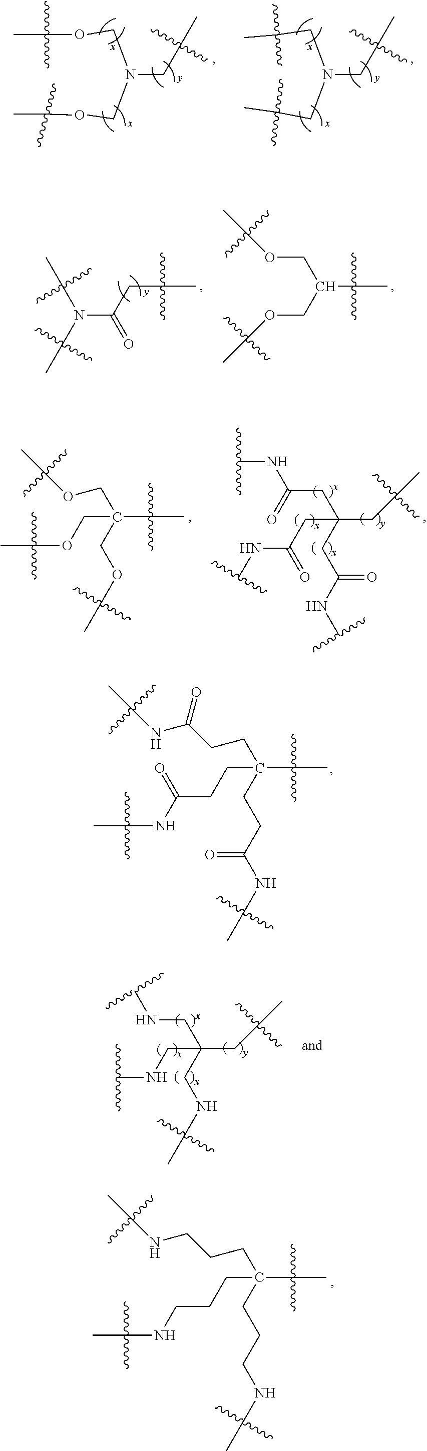
wherein each x and y are independently 1 to 10.

62. The compound of any one of claims 53 to 61, wherein L¹-L² comprises a backbone of 14 or more consecutive atoms (e.g., such as 14 to 50, or 14 to 30 atoms) between Z² or Z⁴ and the branching atom.

63. The compound of any one of claims 53 to 62, wherein L³ comprises a backbone of 10 to 80 consecutive atoms (e.g., such as 12 to 50 atoms).

64. The compound of claim 63, wherein L³ comprises a linking moiety selected from (C₁₀-C₂₀-alkylene (e.g., C₁₂-alkylene), or —(OCH₂CH₂)ₚ—, where p is 11 to 25 (e.g., 3, 7, or 24).

65. The compound of any one of claims 53 to 64, wherein the linker of formula (II) comprises 20 to 100 consecutive atoms.

66. The compound of claim 65, wherein the linker of formula (II) comprises 25 or more consecutive atoms.

67. The compound of claim 65, wherein the linker of formula (II) comprises 30 or more consecutive atoms.

68. The compound of any one of claims 1 to 67, wherein m is 1.

69. The compound of any one of claims 1 to 67, wherein m is at least 2.

70. The compound of claim 69, wherein m is 2 to 20 (e.g., m is 2 to 10).

71. The compound of claim 69, wherein:
m is 20 to 500 (e.g., 20 to 400, 20 to 300, or 20 to 200, or 50 to 500, or 100 to 500); and
L is an α-amino acid polymer (e.g., poly-L-lysine) wherein a multitude of —Ar—Z³-groups are covalently linked to the polymer backbone via sidechain groups (e.g., via conjugation to the sidechain amino groups of lysine residues).

72. The compound of any one of claims 1 to 71, wherein Y is selected from small molecule, dye, fluorophore, monosaccharide, disaccharide, trisaccharide, and chemoselective ligation group or precursor thereof.

73. The compound of any one of claims 1 to 71, wherein Y is a biomolecule.

74. The compound of claim 73, wherein the biomolecule is selected from peptide, protein, polynucleotide, polysaccharide, glycoprotein, lipid, enzyme, antibody, and antibody fragment.

75. The compound of any one of claims 1 to 74, wherein Y is a moiety that specifically binds a target protein

76. The compound of claim 76, wherein the target protein is a membrane bound protein.

77. The compound of claim 76, wherein the target protein is a soluble extracellular protein.

78. The compound of any one of claims 74 to 77, wherein Y is selected from antibody, antibody fragment (e.g., antigen-binding fragment of an antibody), chimeric fusion protein, engineered protein domain, D-protein binder of target protein, aptamer, peptide, and small molecule inhibitor or ligand.

79. A target protein degrading conjugate of formula (XXI): 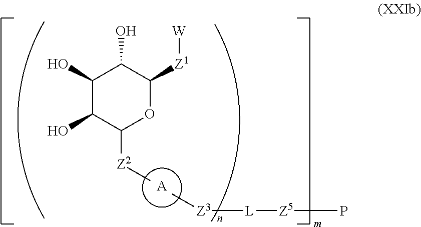
or a prodrug thereof, or pharmaceutically acceptable salt thereof,
wherein:
n is 1 to 3;
m is an average loading of 1 to 10;
L is a linker;
P is a biomolecule that specifically binds the target protein;
Z⁵ is a residual linking moiety resulting from the covalent linkage of a chemoselective ligation group of the linker L to a compatible group of P;
W is a non-hydrolyzable hydrophilic head group;
Z¹ is selected from optionally substituted (C₁-C₃)alkylene and optionally substituted ethenylene;
Z² is selected from O, S, NR²¹ and C(R²²)₂, wherein R²¹ is independently selected from H, and optionally substituted (C₁-C₆)alkyl, and each R²² is independently selected from H, halogen (e.g., F) and optionally substituted (C₁-C₆)alkyl;
A is an optionally substituted cyclic group; and
Z³ is a linking moiety.

80. The conjugate of claim 79, wherein the conjugate is of formula (XXIb):

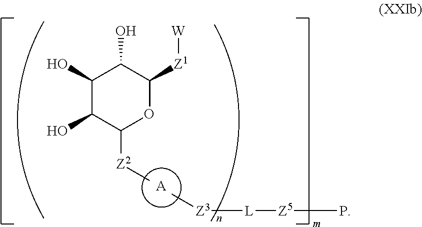

81. The conjugate of claim 79 or 80, wherein Z² is S.

82. The conjugate of claim 79 or 80, wherein Z² is O.

83. The conjugate of claim 79 or 80, wherein Z² is —CH₂— or —CF₂—.

84. The conjugate of any one of claims 79-83, wherein A is optionally substituted aryl or optionally substituted heteroaryl.

85. The conjugate of any one of claims 79-84, wherein A is independently selected from optionally substituted phenyl, optionally substituted pyridyl, optionally substituted biphenyl, optionally substituted naphthalene, optionally substituted triazole and optionally substituted phenylene-triazole.

86. The conjugate of any one of claims 79-85, wherein A is selected from: 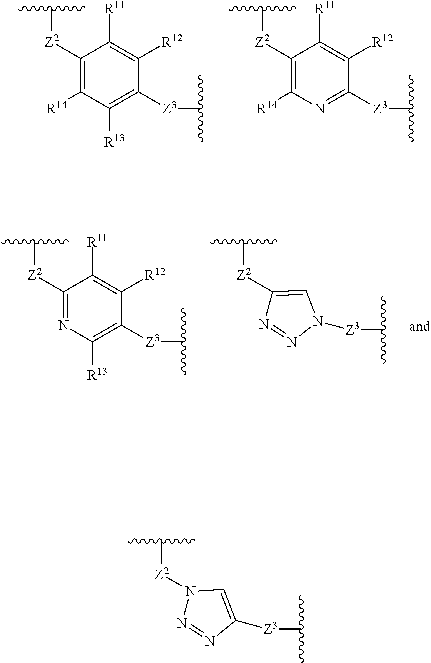
wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
R²⁵ is independently selected from H, and optionally substituted (C₁-C₆)alkyl.

87. The conjugate of any one of claims 84 to 86, wherein A is substituted with at least one OH substituent.

88. The conjugate of claim 86, wherein at least one of R¹¹ to R¹⁴ is OH (e.g., at least two are OH).

89. The conjugate of claim 86, wherein R¹¹ to R¹⁵ are each H.

90. The conjugate of any one of claims 79 to 89, wherein:
Z³ is selected from a covalent bond, —O—, —NR²³—, —NR²³CO—, —CONR²³—, —NR²³CO₂—OCONR²³, —NR²³C(═X¹)NR²³—, —CR²⁴═N—, —CR²⁴═N—X², —N(R²³)SO₂— and —SO₂N(R²³)—, wherein:
X¹ and X² are selected from O, S and NR²³; and
R²³ and R²⁴ are independently selected from H, C₍₁₋₃₎-alkyl (e.g., methyl) and substituted C₍₁₋₃₎-alkyl.

91. The conjugate of any one of claims 79 to 90, wherein Z³ is 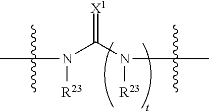
wherein:
X¹ is O or S;
t is 0 or 1; and
each R²³ is independently selected from H, C₍₁₋₃₎-alkyl (e.g., methyl) and substituted C₍₁₋₃₎-alkyl.

92. The conjugate of claim 91, wherein Z³ is —NHC(═O)NH—.

93. The conjugate of any one of claims 79 to 90, wherein -A-Z³— is selected from:

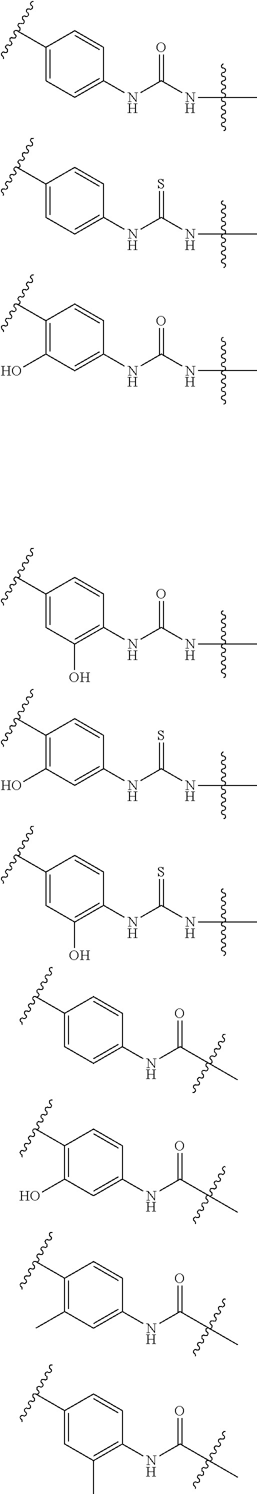

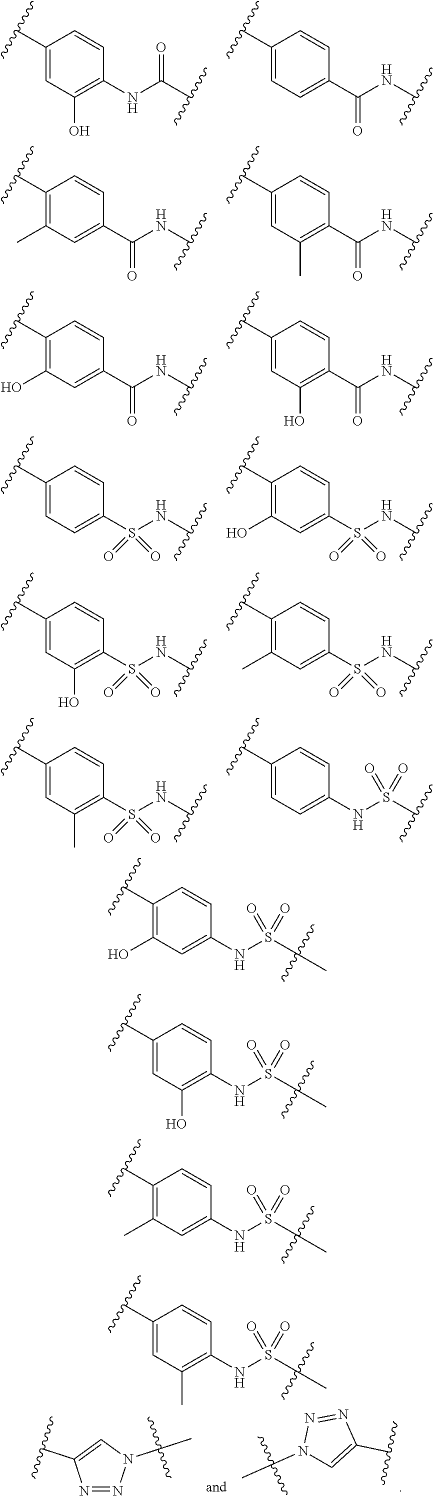

94. The conjugate of any one of claims 79 to 93, wherein the non-hydrolyzable hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —CR¹R²—P═O(OH)₂, —SO₂OH (i.e., —SO₃H), —S(O)OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H, 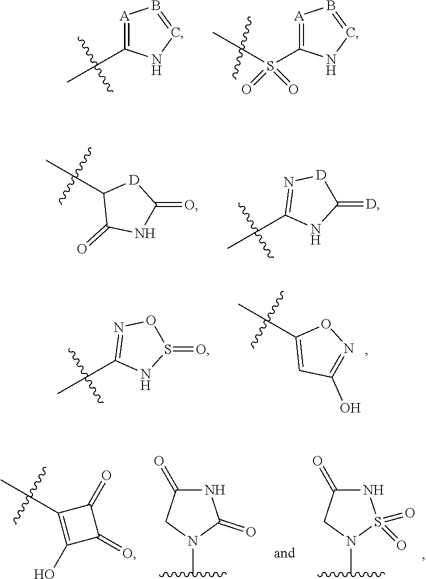
or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

95. The conjugate of claim 94, wherein W is selected from —P═O(OH)₂, —P═S(OH)₂, —P═O(SH)(OH), —P═S(SH)(OH), —COOH and —CH(COOH)₂, or a salt thereof.

96. The conjugate of any one of claims 79 to 95, wherein the conjugate comprises a M6PR binding moiety of one of formula: 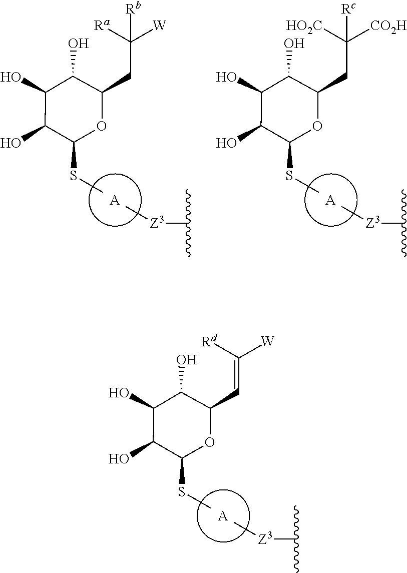
wherein:
W is selected from —P═O(OH)₂, —P═S(OH)₂, —P═O(SH)(OH), —P═S(SH)(OH), and —COOH, or a salt thereof; and
R^{a}, R^{b}, R^{c} and R^{d} are independently H or F.

97. The conjugate of any one of claims 79 to 96, wherein n is 1.

98. The conjugate of any one of claims 79 to 96, wherein n is 2.

99. The conjugate of any one of claims 79 to 96, wherein n is 3.

100. The conjugate of any one of claims 79 to 99, wherein Y is an antibody or antibody fragment that specifically binds the target protein.

101. The conjugate of any one of claims 76 to 99, wherein m is 1 to 8 (e.g., 1 to 7, or 1 to 6).

102. The conjugate of claim 101, wherein m is about 8, about 6, about 5, about 4, about 3 or about 2.

103. The conjugate of any one of claims 79 to 96, wherein n is 1, and m is 1 to 10.

104. The conjugate of claim 103, wherein m is 2 to 8 (e.g., 2 to 6, or 3 to 5).

105. The conjugate of claim 104, wherein m is about 4.

106. The conjugate of any one of claims 79 to 96, wherein n is 2, and m is 1 to 6 (e.g., 2 to 6, or 3 to 5).

107. The conjugate of claim 106, wherein m is about 4.

108. The conjugate of any one of claims 79 to 107, wherein Z⁵ is a residual moiety resulting from the covalent linkage of a thiol-reactive chemoselective ligation group (e.g., maleimide) to one or more cysteine residue(s) of P.

109. The conjugate of any one of claims 79 to 107, wherein Z⁵ is a residual moiety resulting from the covalent linkage of an amine-reactive chemoselective ligation group (e.g., PFP ester or TFP ester) to one or more lysine residue(s) of P.

110. The conjugate of any one of claims 79 to 109, wherein L is a linear linker having a backbone of 16 or more consecutive atoms covalently linking Z³ to P (e.g., a backbone of 16-100, or 20-100 consecutive atoms).

111. The conjugate of any one of claims 79 to 109, wherein L is a branched linker having a backbone of 14 or more consecutive atoms (e.g., such as 14 to 50, or 14 to 30 atoms) between Z² and the branching atom of the linker.

112. The conjugate of any one of claims 79 to 111, wherein the linker L is selected from any one of the structures of Tables 4-5.

113. The conjugate of any one of claims 79 to 112, wherein the conjugate is derived from conjugation of a compound of any one of the structures of Tables 7-9, 12 and 13 and the biomolecule P.

114. The conjugate of claim 113, wherein P is an antibody or antibody fragment.

115. The conjugate of claim 114, wherein the antibody or antibody fragment is an IgG antibody.

116. The conjugate of claim 114 or 115, wherein the antibody or antibody fragment is a humanized antibody.

117. The conjugate of any one of claims 114-116, wherein the antibody or antibody fragment specifically binds to a secreted or soluble protein.

118. The conjugate of any one of claims 114-116, wherein the antibody or antibody fragment specifically binds to a cell surface receptor.

119. A method of internalizing a target protein in a cell comprising a cell surface M6PR, the method comprising contacting a cellular sample comprising the cell and the target protein with an effective amount of a compound according to any one of claims 1 to 78, or a conjugate according to any one of claims 79 to 118, wherein the compound or conjugate specifically binds the target protein and specifically binds the cell surface receptor to facilitate cellular uptake of the target protein.

120. The method of claim 119, wherein the target protein is a membrane bound protein.

121. The method of claim 119, wherein the target protein is an extracellular protein.

122. The method of any one of claims 119 to 121, wherein the compound or conjugate comprises an antibody or antibody fragment (Ab) that specifically binds the target protein.

123. A method of reducing levels of a target protein in a biological system, the method comprising contacting the biological system with an effective amount of a compound according to any one of claims 1 to 78, or a conjugate according to any one of claims 79 to 118, wherein the compound or conjugate specifically binds the target protein and specifically binds a cell surface M6PR of cells in the biological system to facilitate cellular uptake and degradation of the target protein.

124. The method of claim 123, wherein the biological system is a human subject.

125. The method of claim 123, wherein the biological system is an in vitro cellular sample.

126. The method of any one of claims 123 to 125, wherein the target protein is a membrane bound protein.

127. The method of any one of claims 123 to 125, wherein the target protein is an extracellular protein.

128. A method of treating a disease or disorder associated with a target protein, the method comprising: administering to a subject in need thereof an effective amount of a compound according to any one of claims 1 to 78, or a conjugate according to any one of claims 79 to 118, wherein the compound or conjugate specifically binds the target protein.

129. The method of claim 128, wherein the disease or disorder is an inflammatory disease.

130. The method of claim 128, wherein the disease or disorder is an autoimmune disease.

131. The method of claim 128, wherein the disease or disorder is a cancer.
